(19) United States
(12) Patent Application Publication
(10) Pub. No.: US 2018/0125988 A1
(43) Pub. Date: May 10, 2018

(54) TARGETED XTEN CONJUGATE COMPOSITIONS AND METHODS OF MAKING SAME

(71) Applicant: AMUNIX OPERATING INC., Mountain View, CA (US)

(72) Inventors: Fan YANG, San Jose, CA (US); Volker SCHELLENBERGER, Palo Alto, CA (US); Sheng DING, Foster City, CA (US); Desiree THAYER, Burlingame, CA (US); Chia-Wei WANG, Santa Clara, CA (US)

(21) Appl. No.: 15/525,819

(22) PCT Filed: Nov. 11, 2015
(86) PCT No.: PCT/US15/60230

§ 371 Date: May 10, 2017

Related U.S. Application Data
(60) Provisional application No. 62/211,378, filed on Aug. 28, 2015.
(60) Provisional application No. 62/119,483, filed on Feb. 23, 2015.
(60) Provisional application No. 62/078,171, filed on Nov. 11, 2014.

(51) Int. Cl. A61K 47/55; A61K 47/68; C07K 14/00; C12N 15/70; A61P 35/00

(52) U.S. Cl. CPC A61K 47/551; A61K 47/6803; A61K 47/6855; C07K 14/00; C12N 15/70; C12N 9/6421; C07K 2319/01; C07K 2319/30; C07K 2319/50; C07K 2319/70; A61P 35/00

(57) ABSTRACT

The present disclosure provides drug conjugate compositions, and compositions and methods for preparing and using the same. In some embodiments, the present invention relates to targeted conjugate compositions comprising cysteine-containing domains (CCD) linked to targeting moieties, extended recombinant polypeptides (XTEN)and peptide cleavable moieties, with pharmacologically active payload drugs cross-linked to cysteine residues, resulting in compositions that can be cleaved by proteases associated with target tissues. The invention also provides methods of making the targeted conjugate compositions and methods of using the targeted conjugate compositions.

### CROSS-REFERENCE

This application claims the benefit of U.S. Provisional Application No. 62/078,171, filed Nov. 11, 2014; U.S. Provisional Application No. 62/119,483, filed Feb. 23, 2015; and U.S. Provisional Application No. 62/211,378, filed Aug. 28, 2015; all of which are incorporated herein by reference. This application is related to U.S. Provisional Application No. 62/254,076, filed Nov. 11, 2015, which is incorporated herein by reference.

### SEQUENCE LISTING

The instant application contains a Sequence Listing which has been submitted electronically in ASCII format and is hereby incorporated by reference in its entirety. Said ASCII copy, created on Jan. 12, 2016, is named 32808-753.601_SL.txt and is 1,419,218 bytes in size.

### BACKGROUND OF THE INVENTION

Many approved cancer therapeutics are cytotoxic drugs that kill normal cells as well as tumor cells. The therapeutic benefit of these cytotoxic drugs largely depends on tumor cells being more sensitive than normal cells, thereby allowing clinical responses to be achieved using doses that do not result in unacceptable side effects. However, essentially all of these non-specific drugs result in some damage to normal tissues, which often limits treatment.

The use of cytotoxic drugs linked to antibodies or other molecules that bind cell ligands, generally called by the acronym “ADC” (antibody-drug conjugates), are meant to further increase the therapeutic index (or therapeutic window) by selectively delivering the cytotoxic drug to the cancer cell. While the ADCs offer great promise, the numbers of approved drugs remain low, their manufacture is complex and expensive (humanization of murine monoclonals and the large number of mutations typically required to humanise such antibodies), and the pharmacokinetics of many are insufficient; e.g., use of antibody fragments such as scFv in the ADC. Additionally, the size of antibody-based ADCs is a limitation with respect to the ability to of such compositions to penetrate solid tumors or tissues and organs haboring cancer cells.

Extending the half-life a therapeutic agent, whether being a therapeutic protein, peptide or small molecule, often requires specialized formulations or modifications to the therapeutic agent itself. Conventional modification methods such as pegylation, adding to the therapeutic agent an antibody fragment or an albumin molecule, suffer from a number of profound drawbacks. While these modified forms can be prepared on a large scale, these conventional methods are generally plagued by high cost of goods, complex process of manufacturing, and low purity of the final product. Oftentimes, it is difficult, if not impossible, to purify to homogeneity of the target entity. This is particularly true for pegylation, where the reaction itself cannot be controlled precisely to generate a homogenous population of pegylated agents that carry the same number or mass of polyethylene-glycol. Further, the metabolites of these pegylated agents can have sever side effects. For example, PEGylated proteins have been observed to cause renal tubular vacuolation in animal models (Bendele, A., Seely, J., Richey, C., Sennello, G. & Shopp, G. Short communication: renal tubular vacuolation in animals treated with polyethylene-glycol-conjugated proteins. Toxicol. Sci. 1998. 42, 152-157). Renally cleared PEGylated proteins or their metabolites may accumulate in the kidney, causing formation of PEG hydrates that interfere with normal glomerular filtration. In addition, animals and humans can be induced to make antibodies to PEG (Sroda, K. et al. Repeated injections of PEG-PE liposomes generate anti-PEG antibodies. Cell. Mol. Biol. Lett. 2005.10, 37-47).

Thus, there remains a considerable need for anticancer agents that can penetrate and/or attach to tumors or cancerous tissues and deliver cytotoxic compounds to the cancer cells, as well as having sufficient half-life and enhanced selectivity such that the overall therapeutic index is improved.

### SUMMARY OF THE INVENTION

In some aspects, the present invention discloses targeted conjugate compositions comprising one or more extended recombinant polypeptide sequences (XTEN), one or more peptidic cleavage moieties (PCM), one or more targeting moieties (TM), and one or more molecules of a payload drug, wherein the PCM is capable of being cleaved when the conjugate composition is exposed to the protease. The present invention also relates to methods of treatment using the disclosed conjugate compositions in treatment of a disease.

The compositions and methods disclosed herein not only are useful as therapeutics but are also particularly useful as research tools for preclinical and clinical development of a candidate therapeutic agent. In some aspects, the present invention addresses this need by, in part, generating targeted conjugate compositions with payload peptides, proteins and small molecules, as well as targeting moieties that target tissues bearing certain ligands, and that have peptidyl cleave moieties that are capable of being cleaved by proteases when in proximity to the target tissues or target cells. The targeted conjugate compositions are superior in one or more aspects including enhanced terminal half-life, targeted delivery, and reduced toxicity to healthy tissues compared to unconjugated product.

It is specifically contemplated that the cleavable conjugate composition embodiments can exhibit one or more or any combination of the properties disclosed herein. It is further specifically contemplated that the methods of treatment can exhibit one or more or any combination of the properties disclosed herein.

In one aspect, the present disclosure provides a cysteine containing domain (CCD). In some embodiments, the CCD comprises at least 6 amino acid residues, wherein the domain is characterized in that: (a) it has at least one cysteine residue; (b) it has at least one non-cysteine residue, and at least 90%, or at least 91%, or at least 92%, or at least 93%, or at least 94%, or at least 95%, or at least 96%, or at least 97%, or at least 98%, or at least 99%, or 100% of the non-cysteine residues are selected from 3 to 6 types of amino acids selected from the group consisting of glycine (G), alanine (A), serine (S), threonine (T), glutamate (E) and proline (P); (c) no three contiguous amino acids are identical unless the amino acid is cysteine or serine; and (d) no glutamate residue is adjacent to a cysteine residue. In some embodiments, the CCD has between 6 to about 144 amino acid residues and between 1 to about 10 cysteine residues. In some embodiments, the CCD comprises at least 2 cysteine residues, and any two adjacent cysteines are separated by no more than 15 non-cysteine amino acid residues. In some embodiments, at least one cysteine residue is located within 9 amino acid residues from the N- or C-terminus of the CCD. In some embodiments, the CCD sequence has at least 90%, or 91%, or 92%, or 93%, or 94%, or 95%, or 96%, or 97%, or 98%, or 99% identity or is identical to a sequence selected from the sequence set forth in Table 6.

In one aspect, the present disclosure provides a fusion protein comprising any CCD disclosed herein. In some embodiments, the fusion protein comprises the CCD fused to an extended recombinant polypeptide (XTEN), wherein the XTEN is characterized in that: (a) it has a molecular weight that is at least 4-fold, at least 5-fold, at least 6-fold, at least 7-fold, at least 8-fold, at last 10-fold, at least 20-fold, or at least 30-fold greater than the molecular weight of the CCD; (b) it has between 100 to about 1200 amino acids wherein at least 90%, or at least 91%, or at least 92%, or at least 93%, or at least 94%, or at least 95%, or at least 96%, or at least 97%, or at least 98%, or at least 99%, or 100% of the amino acid residues are selected from 4 to 6 types of amino acids selected from the group consisting of glycine (G), alanine (A), serine (S), threonine (T), glutamate (E) and proline (P); (c) it is substantially non-repetitive such that (1) the XTEN sequence contains no three contiguous amino acids that are identical unless the amino acids are serine, (2) at least 90% of the XTEN sequence consists of non-overlapping sequence motifs, each of which comprise 12 amino acid residues, wherein any two contiguous amino acid residues does not occur more than twice in each of the sequence motifs; or (3) the XTEN sequence has an average subsequence score of less than 3; (d) it has greater than 90% random coil formation as determined by GOR algorithm; (e) it has less than 2% alpha helices and 2% beta-sheets as determined by Chou-Fasman algorithm; and (f) it lacks a predicted T-cell epitope when analyzed by TEPITOPE algorithm, wherein the TEPITOPE algorithm prediction for epitopes within the XTEN sequence is based on a threshold score of −9. In some embodiments, the sequence motifs are selected from the group consisting of the sequences set forth in Table 9. In some embodiments, the XTEN has at least 90% sequence identity, or at least 91%, or at least 92%, or at least 93%, or at least 94%, or at least 95%, or at least 96%, or at least 97%, or at least 98%, or at least 99% sequence identity, or is identical to a sequence selected from the group of sequences set forth in Table 10 or Table 11. In some embodiments, the fusion protein further comprises at least a first targeting moiety (TM) wherein the targeting moiety is capable of specifically binding a ligand associated with a target tissue. In some embodiments, the TM is joined to the N-terminus or the C-terminus of the CCD. In some embodiments, the fusion protein is configured from the N-terminus to the C-terminus as: (a) (TM)-(CCD)-(XTEN); or (b) (XTEN)-(CCD)-(TM). In some embodiments, the TM is fused to the CCD recombinantly. In some embodiments, the TM is conjugated to the CCD using a linker sequence selected from the group consisting of the sequences set forth in Table 12. In some embodiments, the ligand of the target tissue is associated with a tumor, a cancer cell, or a tissue with an inflammatory condition. In some embodiments, the fusion protein further comprises one or more drugs or biologically active proteins, wherein each drug or biologically active protein is conjugated to a thiol group of a cysteine residue of the CCD. In some embodiments, the target tissue is a tumor or a cancer cell and the drug is a cytotoxic drug selected from the group consisting of the drugs of Table 14 and Table 15. In some embodiments, the target tissue is a tumor or a cancer cell and the drug is a cytotoxic drug selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A. In some embodiments, the drug is monomethyl auristatin E (MMAE). In some embodiments, the drug is monomethyl auristatin F (MMAF). In some embodiments, the drug is mertansine (DM1). In some embodiments, the target tissue is a tumor or a cancer cell and the biologically active protein is selected from the group consisting of TNFα, IL-12, ranpirnase, human ribonuclease (RNAse), bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin. In some embodiments, the at least first TM is selected from the group consisting of an IgG antibody, a Fab fragment, a F(ab′)2 fragment, a scFv, a scFab, a dAb, a single domain heavy chain antibody, and a single domain light chain antibody. In some embodiments, the at least first targeting moiety is a scFv. In some embodiments, the scFv comprises a VL and a VH sequence of a monoclonal antibody, wherein each VL and VH has at least 90%, or 91%, or 92%, or 93%, or 94%, or 95%, or 96%, or 97%, or 98%, or 99% identity or is identical to a VL and a VH selected from the group consisting of the VL and VH sequences set forth in Table 19, and further comprises a linker sequence selected from the group consisting of the sequences set forth in Table 20 wherein the linker is recombinantly fused between the VL and the VH. In some embodiments, the scFv is configured from the N-terminus to the C-terminus as VH-linker-VL or VL-linker-VH. In some embodiments, the scFv comprises heavy chain CDR segments HCDR1, HCDR2, HCDR3, light chain CDR segments LCDR1, LCDR2, LCDR3, and framework regions (FR) from an antibody selected from the group of antibodies set forth in Table 19, wherein the heavy chain CDR and FR are fused together in the order FR1-HCDR1-FR2-HCDR2-FR3-HCDR3-FR4 and the light chain CDR and FR are fused together in the order FR1-LCDR1-FR2-LCDR2-FR3-LCDR3-FR4, and further comprises a linker sequence selected from the group consisting of the sequences set forth in Table 20 fusing the light chain segments to the heavy chain segments, wherein the scFv is configured from the N-terminus to the C-terminus as VH-linker-VL or VL-linker-VH. In some embodiments, the fusion protein comprises a second scFv wherein the second scFv is identical to the first scFv and the first and the second scFv are recombinantly fused in series by a linker selected from the group consisting of SGGGGS (SEQ ID NO: 1),GGGGS (SEQ ID NO: 2), GGS, and GSP, wherein the scFv are recombinantly fused to the N-terminus or the C-terminus of the CCD. In some embodiments, the fusion protein comprises a second scFv wherein the second scFv is capable of specifically binding a second ligand associated with the target tissue, wherein (i) the second ligand is different from the ligand bound by the first scFv, (ii) the first and the second scFv are recombinantly fused in series by a linker selected from the group consisting of SGGGGS (SEQ ID NO: 1),GGGGS (SEQ ID NO: 2), GGS, and GSP, and (iii) the scFv are recombinantly fused to the N-terminus or the C-terminus of the CCD. In some embodiments, the second scFv comprises a VL and a VH sequence of a monoclonal antibody, wherein each VL and VH has at least 90%, or 91%, or 92%, or 93%, or 94%, or 95%, or 96%, or 97%, or 98%, or 99% identity or is identical to a VL and a VH selected from the group consisting of the VL and VH sequences set forth in Table 19, and further comprises a linker sequence selected from the group consisting of the sequences set forth in Table 20 wherein the linker is recombinantly fused between the VL and the VH. In some embodiments, the second scFv is configured from the N-terminus to the C-terminus as VH-linker-VL or VL-linker-VH. In some embodiments, the second scFv comprises heavy chain CDR segments HCDR1, HCDR2, HCDR3, light chain CDR segments LCDR1, LCDR2, LCDR3, and the associated framework regions (FR) from an antibody selected from the group of antibodies set forth in Table 20, wherein the heavy chain CDR and FR segments are fused together in the order FR1-HCDR1-FR2-HCDR2-FR3-HCDR3-FR4 and the light chain CDR and FR segments are fused together in the order FR1-LCDR1-FR2-LCDR2-FR3-LCDR3-FR4, and further comprises a linker sequence selected from the group consisting of the sequences set forth in Table 20 fusing the light chain segments to the heavy chain segments. In some embodiments, the at least first TM is selected from the group consisting of folate, luteinizing-hormone releasing hormone (LHRH) agonist, asparaginylglycylarginine (NGR), and arginylglycylaspartic acid (RGD). In some embodiments, the at least first TM is non-proteinaceous. In some embodiments, the at least first TM is folate. In some embodiments, (a) the target tissue has an inflammatory condition; (b) the drug is selected from the group consisting of dexamethasone, indomethacin, prednisolone, betamethasone dipropionate, clobetasol propionate, fluocinonide, flurandrenolide, halobetasol propionate, diflorasone diacetate, and desoximetasone; and (c) the targeting moiety is a scFv derived from a monoclonal antibody capable of specifically binding a ligand selected from the group consisting of TNF, IL-1 receptor, IL-6 receptor, a4 integrin subunit, CD20, and IL-21 receptor. In some embodiments, the scFv comprises a VL and a VH sequence of a monoclonal antibody, wherein each VL and VH has at least 90%, or 91%, or 92%, or 93%, or 94%, or 95%, or 96%, or 97%, or 98%, or 99% identity or is identical to a VL and a VH selected from the group consisting of the VL and VH sequences set forth in Table 19, and further comprises a linker sequence selected from the group consisting of the sequences set forth in Table 20 wherein the linker is recombinantly fused between the VL and the VH. In some embodiments, the fusion protein further comprises a peptidic cleavage moiety (PCM) wherein the PCM is a capable of being cleaved by one, two, or more mammalian proteases. In some embodiments, the fusion protein further comprises a peptidic cleavage moiety (PCM), wherein the PCM is a capable of being cleaved by one, two, or more mammalian proteases, and wherein the fusion protein is configured from the N-terminus to the C-terminus as: (a) (TM)-(CCD)-(PCM)-(XTEN); (b) (XTEN)-(PCM)-(CCD)-(TM); (c) (XTEN)-(PCM)-(TM)-(CCD); or (d) (CCD)-(TM)-(PCM)-(XTEN). In some embodiments, the fusion protein further comprises a second XTEN identical to the first XTEN wherein the first and the second XTEN are both conjugated to the N- or C-terminus of the PCM using a trimeric cross-linker. In some embodiments, the PCM comprises a peptide sequence having at least 90% sequence identity or is identical to a sequence selected from the group of sequences set forth in Table 8. In some embodiments, the mammalian protease is colocalized with the target tissue. In some embodiments, the mammalian protease is an extracellular protease secreted by the target tissue or is a component of a tumor extracellular matrix. In some embodiments, the mammalian protease is selected from the group consisting of proteases set forth in Table 7. In some embodiments, the mammalian protease is selected from the group consisting of meprin, neprilysin (CD10), PSMA, BMP-1, ADAMS, ADAMS, ADAM10, ADAM12, ADAM15, ADAM17 (TACE), ADAM19, ADAM28 (MDC-L), ADAM with thrombospondin motifs (ADAMTS), ADAMTS1, ADAMTS4, ADAMTS5, MMP-1 (Collagenase 1), MMP-2 (Gelatinase A), MMP-3 (Stromelysin 1), MMP-7 (matrilysin 1), MMP-8 (collagenase 2), MMP-9 (Gelatinase B), MMP-10 (stromelysin 2), MMP-11(stromelysin 3), MMP-12 (macrophage elastase), MMP-13 (collagenase 3), MMP-14 (MT1-MMP), MMP-15 (MT2-MMP), MMP-19, MMP-23 (CA-MMP), MMP-24 (MT5-MMP), MMP-26 (Matrilysin 2), MMP-27 (CMMP), legumain, cathepsin B, cathepsin C, cathepsin K, cathepsin L, cathepsin S, cathespin X, cathepsin D, cathepsin E, secretase, urokinase (uPA), tissue-type plasminogen activator (tPA), plasmin, thrombin, prostate-specific antigen (PSA, KLK3), human neutrophil elastase (HNE), elastase, tryptase, Type II transmembrane serine proteases (TTSPs), DESC1, hepsin (HPN), matriptase, natriptase-2, TMPRSS2, TMPRSS3, TMPRSS4 (CAP2), fibroblast activation protein (FAP), kallikrein-related peptidase (KLK family), KLK4, KLK5, KLK6, KLK7, KLK8, KLK10, KLK11, KLK13, and KLK14. In some embodiments, upon performing a conjugation reaction between the drug molecule and the cysteine residues of the CCD of the fusion protein, a heterogeneous population of conjugate products is obtained wherein fully conjugated CCD-drug conjugate product is capable of achieving a peak separation ≥6 wherein: a) the fusion protein comprises a polypeptide having 600 or more cumulative amino acid residues comprising a CCD with between 3 to 9 cysteine residues; b) the heterogeneous conjugate products have a mixture of at least 1, 2, and 3 or more payloads linked to the CCD; and c) the conjugation products are analyzed under reversed-phase HPLC chromatography conditions. In some embodiments, the CCD is a sequence of Table 6 having 3 cysteine residues and the fusion protein has at least 800 cumulative amino acid residues. In some embodiments, the CCD is a sequence of Table 6 having 9 cysteine residues and the fusion protein has at least 800 cumulative amino acid residues. In some embodiments, upon cleavage of the PCM by the target tissue protease, the XTEN is released from the fusion protein, wherein the targeting moiety and the CCD with linked drug or biologically active protein remain joined together as a released targeted composition. In some embodiments, the molecular weight of the released targeted composition has a molecular weight that is at least 4-fold, at least 5-fold, at least 6-fold, at least 7-fold, at least 8-fold, at least 10-fold less compared to the fusion protein that is not cleaved. In some embodiments, the hydrodynamic radius of the released targeted composition is at least 4-fold, at least 5-fold, at least 6-fold, at least 7-fold, at least 8-fold, at least 10-fold less compared to the fusion protein that is not cleaved. In some embodiments, the released targeted composition has a binding affinity that is at least about 2-fold, 3-fold, 4-fold, 5-fold, 6-fold, 7-fold, 8-fold, 9-fold, 10-fold, 20-fold, 30-fold, 40-fold, or 100-fold greater for the target tissue ligand compared to the fusion protein that is not cleaved. In some embodiments, the released targeted composition has a binding affinity constant (K_{d}) for the ligand of less than about 10⁻⁴ M, or less than about 10⁻⁵ M, or less than about 10⁻⁶ M, or less than about 10⁻⁷ M, or less than about 10⁻⁸M, or less than about 10⁻⁹ M, or less than about 10⁻¹⁰ M, or less than about 10⁻¹¹ M, or less than about 10⁻¹² M. In some embodiments, the binding affinity is measured in an in vitro ELISA assay. In some embodiments, the cytotoxicity of the released targeted composition is at least about 2-fold, 3-fold, 4-fold, 5-fold, 6-fold, 7-fold, 8-fold, 9-fold, 10-fold, 20-fold, 30-fold, 40-fold, or 100-fold greater against a target cell bearing the ligand in an in vitro mammalian cell cytotoxicity assay compared to the cytotoxicity of the fusion protein that is not cleaved, wherein cytotoxicity is determined by calculation of IC₅₀. In some embodiments, the released targeted composition inhibits growth of target cells bearing the ligand by at least 20%, or at least 40%, or at least 50% more in an in vitro mammalian cell cytotoxicity assay compared to the inhibition of growth by the fusion protein that is not cleaved when said growth inhibition is determined between 24-72 hours after exposure to the released targeted composition or the fusion protein under comparable conditions. In some embodiments, after administration of a bolus dose of a therapeutically effective amount of the fusion protein to a subject having a targeted tissue bearing the ligand and a colocalized protease capable of cleaving the PCM, the released targeted composition released by the protease is capable of accumulating in the target tissue to a concentration that is at least about 2-fold, 3-fold, 4-fold, 5-fold, 6-fold, 7-fold, 8-fold, 9-fold, 10-fold, 20-fold, 30-fold, 40-fold, or 100-fold greater compared to the fusion protein that is not cleaved. In some embodiments, the targeted tissue is a tumor. In some embodiments, the administration results in a reduction of volume of the tumor of at least 10%, or at least 20%, or at least 30%, or at least 40%, or at least 50% at 7 to 21 days after administration. In some embodiments, the administration results in at least 10%, or at least 20%, or at least 30%, or at least 40%, or at least 50% greater reduction of volume of the tumor at 7-21 days after administration compared to a fusion protein that does not comprise the PCM and is administered at a comparable dose. In some embodiments, the subject is selected from the group consisting of mouse, rat, rabbit, monkey, and human.

In one aspect, the present disclosure provides a targeted conjugate composition. In some embodiments, the targeted conjugate composition is selected from the group consisting of the conjugates of Table 5. In some embodiments, the composition is configured from the N-terminus to the C-terminus as: (a) (TM)-(CCD)-(PCM)-(XTEN); or (b) (XTEN)-(PCM)-(CCD)-(TM); wherein a drug molecule is linked to each cysteine residue of the CCD.

In some embodiments, the targeted conjugate composition comprises (a) a construct of Table 5 comprising an amino acid sequence of the construct, or (b) a variant construct comprising a variant sequence that is at least 90% identical to the amino acid sequence of the construct, wherein the construct has a structure of Formula I:

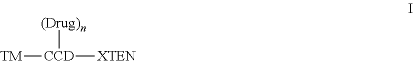

wherein n is an integer equal to the number of cysteine residues of the CCD.

In some embodiments, the targeted conjugate composition comprises (a) a construct of Table 5 comprising an amino acid sequence of the construct, or (b) a variant construct comprising a variant sequence that is at least 90% identical to the amino acid sequence of the construct wherein the construct has a structure of Formula II:

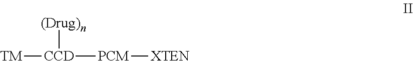

wherein n is an integer equal to the number of cysteine residues of the CCD.

In some embodiments, the targeted conjugate composition comprises (a) a construct of Table 5 comprising an amino acid sequence of the construct, or (b) a variant construct comprising a variant sequence that is at least 90% identical to the amino acid sequence of the construct wherein the construct has a structure of Formula III:

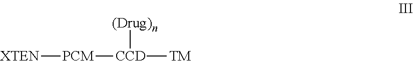

wherein n is an integer equal to the number of cysteine residues of the CCD.

In some embodiments, the targeted conjugate composition comprises (a) a construct of Table 5 comprising an amino acid sequence of the construct, or (b) a variant construct comprising a variant sequence that is at least 90% identical to the amino acid sequence of the construct wherein the construct has a structure of Formula IV:

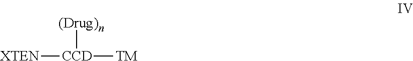

wherein n is an integer equal to the number of cysteine residues of the CCD.

In some embodiments, the targeted conjugate composition is configured according to the structure of Formula I:

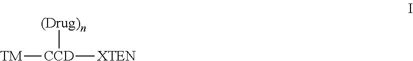

wherein (a) the TM is an scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the CCD is selected from the group consisting of the CCD of Table 6; (c) the XTEN has at least 90%, or 91%, or 92%, or 93%, or 94%, or 95%, or 96%, or 97%, or 98%, or 99% identity or is identical to a sequence set forth in Table 10; and (d) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula II:

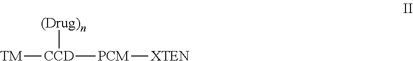

wherein (a) the TM is an scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the CCD is selected from the group consisting of the CCD of Table 6; (c) the PCM is selected from the group consisting of the sequences set forth in Table 8; (d) the XTEN has at least 90%, or 91%, or 92%, or 93%, or 94%, or 95%, or 96%, or 97%, or 98%, or 99% identity or is identical to a sequence set forth in Table 10; and (e) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula III:

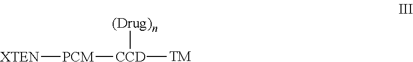

wherein (a) the TM is an scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the CCD is selected from the group consisting of the CCD of Table 6; (c) the PCM is selected from the group consisting of the sequences set forth in Table 8; (d) the XTEN has at least 90%, or 91%, or 92%, or 93%, or 94%, or 95%, or 96%, or 97%, or 98%, or 99% identity or is identical to a sequence set forth in Table 10; and (e) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula IV:

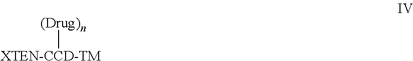

wherein (a) the TM is an scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the CCD is selected from the group consisting of the CCD of Table 6; (c) the XTEN has at least 90%, or 91%, or 92%, or 93%, or 94%, or 95%, or 96%, or 97%, or 98%, or 99% identity or is identical to a sequence set forth in Table 10; and (d) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula V:

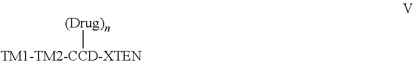

wherein (a) the TM1 is a first scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the TM2 is a second scFv, different from the first scFv, wherein the TM2 comprises a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences in Table 20 wherein the linker is recombinantly fused between the VL and the VH; (c) the CCD is selected from the group consisting of the CCD of Table 6; (d) the XTEN has at least 90%, or 91%, or 92%, or 93%, or 94%, or 95%, or 96%, or 97%, or 98%, or 99% identity or is identical to a sequence set forth in Table 10; and (e) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula VI:

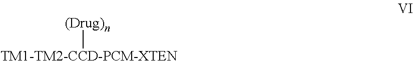

wherein (a) the TM1 is a first scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the TM2 is a second scFv, different from the first scFv, wherein the TM2 comprises a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences in Table 20 wherein the linker is recombinantly fused between the VL and the VH; (c) the CCD is selected from the group consisting of the CCD of Table 6; (d) the PCM is selected from the group consisting of the PCM of Table 8; (e) the XTEN has at least 90%, or 91%, or 92%, or 93%, or 94%, or 95%, or 96%, or 97%, or 98%, or 99% identity or is identical to a sequence set forth in Table 10; and (f) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula VIII:

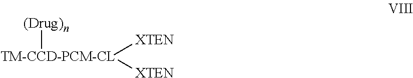

wherein (a) the TM is a scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the CCD is selected from the group consisting of the CCD of Table 6; (c) the PCM is selected from the group consisting of the PCM of Table 8; (d) the CL is a cross-linker selected from the group consisting of the cross-linkers of Table 25; (e) the XTEN has at least 90%, or 91%, or 92%, or 93%, or 94%, or 95%, or 96%, or 97%, or 98%, or 99% identity or is identical to a sequence set forth in Table 10; and (f) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD.

In some embodiments, the targeted conjugate composition is configured according to the structure of Formula X:

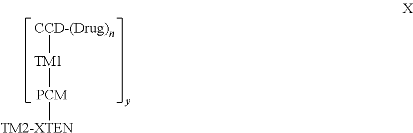

wherein (a) the TM1 is a first scFv comprising a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; (b) the TM2 is a second scFv, different from the first scFv, wherein the TM2 comprises a VL and a VH sequence, wherein each VL and VH has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences in Table 20 wherein the linker is recombinantly fused between the VL and the VH; (c) the CCD is selected from the group consisting of the CCD of Table 6; (d) the PCM is selected from the group consisting of the PCM of Table 8; (e) the XTEN is a cysteine-engineered XTEN having at least 90%, or 91%, or 92%, or 93%, or 94%, or 95%, or 96%, or 97%, or 98%, or 99% identity or is identical to a sequence set forth in Table 11; (f) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is an integer equal to the number of cysteine residues of the CCD; and (g) y is an integer equal to the number of cysteine residues of the XTEN.

In one aspect, the present disclosure provides a pharmaceutical composition. In some embodiments, the pharmaceutical composition comprises a fusion protein in accordance with any of the various embodiments disclosed herein, including with regard to any of the various aspects of the disclosure. In some embodiments, the pharmaceutical composition comprises a targeted conjugate composition in accordance with any of the various embodiments disclosed herein, including with regard to any of the various aspects of the disclosure. In some embodiments, the pharmaceutical composition is for treatment of a disease in a subject wherein the disease is selected from the group consisting of breast cancer, ER/PR+ breast cancer, Her2+ breast cancer, triple-negative breast cancer, liver carcinoma, lung cancer, non-small cell lung cancer, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, pancreatic cancer, leukemia, acute myeloid leukemia (AML), chronic myeloid leukemia (PCML), acute lymphocytic leukemia (ALL), chronic lymphocytic leukemia (CLL), T-cell acute lymphoblastic leukemia, lymphoblastic disease, multiple myeloma, Hodgkin's lymphoma, non-Hodgkin's lymphoma, acne vulgaris, asthma, autoimmune diseases, autoinflammatory diseases, celiac disease, chronic prostatitis, glomerulonephritis, hypersensitivity reaction, inflammatory bowel disease, Crohn's disease, pelvic inflammatory disease, reperfusion injury, rheumatoid arthritis, sarcoidosis, transplant rejection, vasculitis, psoriasis, fibromyalgia, irritable bowel syndrome, lupus erythematosis, osteoarthritis, scleroderma, and ulcerative colitis. In some embodiments, the pharamaceutical composition is for use in a pharmaceutical regimen for treatment of the subject, said regimen comprising the pharmaceutical composition. In some embodiments, the pharmaceutical regimen further comprises the step of determining the amount of pharmaceutical composition needed to achieve a beneficial effect in the subject having the disease.

In one aspect, the present disclosure provides a method of treating a disease in a subject. In some embodiments, the method comprises a regimen of administering one, or two, or three, or four or more therapeutically effective doses of a pharmaceutical composition in accordance with any of the various embodiments disclosed herein, including with regard to any of the various aspects of the disclosure. In some embodiments, the disease is selected from the group consisting of breast cancer, ER/PR+ breast cancer, Her2+ breast cancer, triple-negative breast cancer, liver carcinoma, lung cancer, non-small cell lung cancer, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, and pancreatic cancer. In some embodiments, the administered pharmaceutical composition comprises a targeting moiety wherein the targeting moiety has specific binding affinity for a tumor of the disease. In some embodiments, the administered pharmaceutical composition comprises a targeting moiety wherein the targeting moiety has specific binding affinity for a target selected from the group of targets set forth in Table 2, Table 3, Table 4, Table 18, and Table 19. In some embodiments, the administration results in at least a 10%, or 20%, or 30%, or 40%, or 50%, or 60%, or 70%, or 80%, or 90% greater improvement of at least one, two, or three parameters associated with a cancer compared to an untreated subject wherein the parameters are selected from the group consisting of time-to-progression of the cancer, time-to-relapse, time-to-discovery of local recurrence, time-to-discovery of regional metastasis, time-to-discovery of distant metastasis, time-to-onset of symptoms, pain, body weight, hospitalization, time-to-increase in pain medication requirement, time-to-requirement of salvage chemotherapy, time-to-requirement of salvage surgery, time-to-requirement of salvage radiotherapy, time-to-treatment failure, and time of survival. In some embodiments, the administered doses result in a decrease in the tumor size in the subject. In some embodiments, the decrease in tumor size is at least 10%, or at least 20%, or at least 30%, or at least 40%, or at least 50% or greater. In some embodiments, the decrease in tumor size is achieved within at least about 10 days, at least about 14 days, at least about 21 days after administration, or at least about 30 days after administration. In some embodiments, the administered doses result in tumor stasis in the subject. In some embodiments, tumor stasis is achieved within at least about 10 days, at least about 14 days, at least about 21 days after administration, or at least about 30 days after administration. In some embodiments, the regimen comprises administration of the therapeutically effective dose every 7 days, or every 10 days, or every 14 days, or every 21 days, or every 30 days. In some embodiments, the pharmaceutical composition is administered using a therapeutically effective dose regimen in a subject, wherein the therapeutically effective dose regimen results in a growth inhibitory effect on a tumor cell bearing a target selected from the group of targets set forth in Table 2, Table 3, Table 4, Table 18, and Table 19. In some embodiments, the fusion protein or the targeted conjugate composition of the pharmaceutical composition exhibits a terminal half-life that is longer than at least at least about 72 h, or at least about 96 h, or at least about 120 h, or at least about 144 h, or at least about 10 days, or at least about 21 days, or at least about 30 days when administered to a subject.

In one aspect, the present disclosure provides a method of reducing a frequency of treatment in a subject with a cancer tumor. In some embodiments, the method comprises administering a pharmaceutical composition to the subject using a therapeutically effective dose regimen for the pharmaceutical composition. The pharmaceutical composition can be any pharmaceutical composition in accordance with any of the various embodiments disclosed herein, including with regard to any of the various aspects of the disclosure. In some embodiments, the administration results in a decrease in tumor size in the subject, wherein the decrease in tumor size is at least 10%, or at least 20%, or at least 30%, or at least 40%, or at least 50% or greater. In some embodiments, the regimen resulting in a decrease in cancer tumor size is administration of a therapeutically effective dose of the pharmaceutical composition every 7 days, or every 10 days, or every 14 days, or every 21 days, or every 30 days, or monthly. In some embodiments, the regimen resulting in a decrease in cancer tumor size has dosing intervals in a subject that are 3-fold, or 4-fold, or 5-fold, or 6-fold, or 7-fold, or 8-fold, or 9-fold, or 10-fold greater compared to the therapeutically-effective dose regimen of the corresponding payload drug not linked to the conjugate composition.

In one aspect, the present disclosure provides a method of treating a cancer cell in vitro. In some embodiments, the method comprises administering to a cell culture of a cancer cell an effective amount of a fusion protein in accordance with any of the various embodiments disclosed herein, including with regard to any of the various aspects of the disclosure, wherein the administration results in a cytotoxic effect to the cancer cell. In some embodiments, the method comprises administering to a cell culture of a cancer cell an effective amount of a targeted conjugate composition in accordance with any of the various embodiments disclosed herein, including with regard to any of the various aspects of the disclosure, wherein the administration results in a cytotoxic effect to the cancer cell. In some embodiments, the cancer cell has a target for which the TM of the conjugate composition has binding affinity. In some embodiments, the target is selected from the group consisting of the targets set forth in Table 2, Table 3, Table 4, Table 18, and Table 19. In some embodiments, the culture comprises a protease capable of cleaving the PCM of the conjugate composition. In some embodiments, the cancer cell is selected from the group consisting of the cell lines of Table 18. In some embodiments, the cytotoxic effect of the conjugate composition is greater compared to that seen using a cancer cell that does not have the ligand for the TM of the conjugate composition.

In one aspect, the present disclosure provides an isolated nucleic acid. In some embodiments, the isolated nucleic acid comprises (a) a polynucleotide sequence encoding a fusion protein in accordance with any of the various embodiments disclosed herein, including with regard to any of the various aspects of the disclosure, and/or (b) a complement of the polynucleotide according to (a).

In one aspect, the present disclosure provides an expression vector. In some embodiments, the expression vector comprises a polynucleotide according to any of the various aspects and embodiments disclosed herein, and a recombinant regulatory sequence operably linked to the polynucleotide sequence.

In one aspect, the present disclosure provides a host cell. In some embodiments, the host cell comprises an expression vector according to any of the various aspects and embodiments disclosed herein. In some embodiments, the host cell is a prokaryote. In some embodiments, the host cell is E. coli.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the invention may be further explained by reference to the following detailed description and accompanying drawings that sets forth illustrative embodiments

FIG. 1 shows schematics of XTEN suitable for conjugation with payloads. FIG. 1A shows unmodified XTEN of different length. FIG. 1B shows a cysteine-engineered XTEN with an internal cysteine with a thiol side chain; below is an XTEN with a reactive N-terminal amino group; below is an XTEN with an N-terminal cysteine with a thiol reactive group. FIG. 1C shows cysteine-engineered XTEN with multiple internal cysteines (left) and lysine-engineered XTEN with multiple reactive amino-groups (right). FIG. 1D shows three variations of XTEN with engineered thiol and amino groups.

FIG. 2 shows a conjugation reaction utilizing NHS-esters and their water soluble analogs sulfo-NHS-esters) reacting with a primary amino group to yield a stable amide XTEN-payload product.

FIG. 3 shows various conjugation reactions. FIG. 3A shows a conjugation reaction utilizing thiol groups and an N-maleimide. The maleimide group reacts specifically with sulfhydryl groups when the pH of the reaction mixture is between pH 6.5 and 7.5, forming a stable thioether linkage that is not reversible. FIG. 3B shows a conjugation reaction utilizing haloacetyls. The most commonly used haloacetyl reagents contain an iodoacetyl group that reacts with sulfhydryl groups at physiological pH. The reaction of the iodoacetyl group with a sulfhydryl proceeds by nucleophilic substitution of iodine with a thiol producing a stable thioether linkage in the XTEN-payload. FIG. 3C shows a conjugation reaction utilizing pyridyl disulfides. Pyridyl disulfides react with sulfhydryl groups over a broad pH range (the optimal pH is 4-5) to form disulfide bonds linking XTEN to payloads.

FIG. 4 (in FIG. 4A and FIG. 4B) shows a conjugation reaction utilizing zero-length cross-linkers wherein the cross-linkers are used to directly conjugate carboxyl functional groups of one molecule (such as a payload) to the primary amine of another molecule (such as an XTEN).

FIG. 5 shows a click conjugation reaction utilizing the Huisgen 1,3-dipolar cycloaddition of alkynes to azides to form 1,4-disubsituted-1,2,3-triazoles, as shown.

FIG. 6 shows a conjugation reaction using thio-ene based chemistry that may proceed by free radical reaction, termed thiol-ene reaction, or anionic reaction, termed thiol Michael addition.

FIG. 7 shows a conjugation reaction utilizing chemistry based on reactions between hydrazides and aldehydes, resulting in the illustrated hydrazone linkage in the XTEN-payload.

FIG. 8 shows conjugation reactions utilizing enzymatic ligation. FIG. 8A: Transglutaminases are enzymes that catalyze the formation of an isopeptide bond between the γ-carboxamide group of glutamine of a payload peptide or protein and the ε-amino group of a lysine in a lysine-engineered XTEN (or an N-terminal amino group), thereby creating inter- or intramolecular cross-links between the XTEN and payload. FIG. 8B shows enzymatically-created XTEN-payload compositions utilizing the sortase A transpeptidase enzyme from Staphylococcus aureus to catalyze the cleavage of a short 5-amino acid recognition sequence LPXTG (SEQ ID NO: 3) between the threonine and glycine residues of Proteinl that subsequently transfers the acyl-fragment to an N-terminal oligoglycine nucleophile of Proteinl. By functionalizing the Protein2 to contain an oligoglycine, the enzymatic conjugation of the two proteins is accomplished in a site-specific fashion to result in the desired XTEN-payload composition. Figure discloses SEQ ID NOS 768-769, respectively, in order of appearance.

FIG. 9 shows various XTEN-cross-linker precursor segments that are used as reactants to link to targeting moieties, payloads or to other XTEN reactants. FIG. 9A is intended to show that the 1B represents the remaining reactive group of the precursors on the right. FIG. 9B shows similar reactive precursors with either multiple (left) or single (right) payload A molecules conjugated to the XTEN.

FIG. 10 shows exemplary permutations of XTEN-cross-linker precursor segments with two reactive groups of cross-linkers or reactive groups of an incorporated amino acid that are used as reactants to link to payloads or to other XTEN reactants. The 1B and 2B represent reactive groups that will, in other figures, react with a like-numbered reactive group; 1 with 1 and 2 with 2, etc.

FIG. 11 is intended to show examples of various reactants and the nomenclature for configurations illustrated elsewhere in the Drawings. FIG. 11A shows various forms of reactive XTEN segment precursors, each with a different reactive group on the N-terminus. FIG. 11B shows various cross-linkers with 2, 3 or 4 reactive groups. In the first case, the divalent cross-linker is a heterofunctional linker that reacts with two different types of reactive groups, represented by “2” and “1”. The remaining three represent divalent, trivalent, and tetravalent cross-linkers of the same reactive group. FIG. 11C illustrates the nomenclature of the reaction products of two XTEN segment precursors. In the top version, a 1A was reacted with a 1B to create a dimeric XTEN linked at the N-termini, with the residue of the cross-linker indicated by 1AR-1BR, while the bottom version is also a dimeric XTEN linked at the N-termini, with the residue of the cross-linker indicated by 2AR-2BR. However, the same approach can also be used to conjugate targeting moieties to XTEN or CCD or to conjugate payload drugs to CCD.

FIG. 12 illustrates the creation of various XTEN precursor segments. FIG. 12A shows the steps of making an XTEN polypeptide, followed by reaction of the N-terminus with the cross-linker with 2B-1A cross-linker, with the 1A reacting with the N-terminal 1B (e.g., an alpha amino acid) to create the XTEN precursor 2 with the reactive group 2B. FIG. 12B shows the sequential addition of two cross-linkers with 2A reactive groups to 2B reactive groups of the XTEN, resulting in XTEN precursor 4, which is then reacted with a cross-linker at the N-terminus between a reactive 1B and the 1A of a cross-linker, resulting in XTEN precursor 5, with reactive groups 4B and 3B. In such case, the XTEN-precursors 5 then could serve as a backbone reactant to conjugate with two targeted conjugate fusion proteins to 3B and a targeting moiety to 4B.

FIG. 13 illustrates various configurations of bispecific conjugates with two payloads. FIG. 13A illustrates configurations with one molecule each of two payloads, while FIG. 13B illustrates various configurations with multiple copies of one or both payloads.

FIG. 14 shows examples of conjugates comprising a targeting moiety, XTEN, and a CCD with linked payloads. Targeting moieties can be peptides, peptoids, or receptor ligands. FIG. 14A shows a single fusion protein of a CCD-XTEN conjugated to the targeting moiety. The CCD has 3 payloads conjugated to the cysteine residues. FIG. 14B shows a conjugate of a TM conjugated to the terminus of two CCD-XTEN fusion proteins (which could include PCM-TM-CCD-drug payloads) in which the payloads are conjugated to cysteine residues of the CCD.

FIG. 15 shows an example of the creation of a combinatorial CCD-PCM-XTEN conjugate library. Payloads A, B, C are conjugated to CCD-PCM-XTEN carrying reactive group 1A, resulting in one set of CCD-PCM-XTEN-precursor segments. Payloads E, F, and G are conjugated to CCD-PCM-XTEN carrying reactive group 1B, resulting in a second set of CCD-PCM-XTEN-precursor segments. These segments are subjected to combinatorial conjugation and then are purified from reactants. This enables the formation of combinatorial products that can be immediately subjected to in vitro and in vivo testing. In this case, reactive groups 1A and 1B are the alpha-amino groups of XTEN with or without a bispecific cross-linker. In one example, the 1A is an azide and 1B is an alkyne or vice versa, while the payloads are attached to XTEN via thiol groups in XTEN. The PCM domain is optional in the CCD-PCM-XTEN molecules shown.

FIG. 16 shows an example of the creation of a combinatorial CCD-PCM-XTEN conjugate library that optimizes the ratio between two payloads. Each library member carries a different ratio of payload A and payload E. The PCM domain is optional in the CCD-PCM-XTEN molecules shown. After testing, the desireable candidates incorporated into targeted conjugate compositions.

FIG. 17 shows an example of the creation of a combinatorial CCD-PCM-XTEN conjugate library that creates combinations of targeting moieties and payloads. The targeting moieties are conjugated to CCD-PCM-XTEN carrying reactive group 1A. Payloads E, F, and G are conjugated to CCD-PCM-XTEN carrying reactive group 1B. These segments are subjected to combinatorial conjugation, enabling the formation of combinatorial products where each library member comprises targeting moieties and payloads. All CCD-PCM-XTEN segments carrying payloads and conjugation groups can be purified as combinatorial products that can be immediately subjected to in vitro and in vivo testing. The PCM domain is optional in the CCD-PCM-XTEN molecules shown. After testing, the desireable candidates are incorporated into targeted conjugate compositions.

FIG. 18 shows schematic examples of targeted conjugate compositions interacting with a target cell. FIG. 18A shows an example in which the XTEN remains fused to the CCD and targeting moiety as a fusion protein and binds to to the target receptor that is over-expressed on many cancer cells. Receptor binding results in internalization followed by proteolytic breakdown and the intracellular liberation of Payload A, which is toxic to the cell. FIG. 18B shows a construct design in which the XTEN has been released by cleavage of the PCM and the resulting fragment comprising the targeting moiety and the CCD with linked payloads binds to the target receptor that is over-expressed on many cancer cells. Receptor binding results in internalization followed by proteolytic break down and the intracellular liberation of Payload A, which is toxic to the cell.

FIG. 19 shows the complete purification process of a CCD-XTEN construct, as described in Example 7. FIG. 19A shows a SDS-PAGE analysis of fraction of CCD-XTEN after cation exchange capture step. The materials per lane are: Lane 1: Marker; Lane 2: Cation exchange column load; Lane 3-5: Cation exchange column flow through/wash fractions 1-3; Lane 6: Cation exchange column elution; Lane 7: Cation exchange strip. FIG. 19B shows SDS-PAGE analysis of anion exchange polishing step fractions. The materials per lane are: Lane 1: Marker; Lane 2: Anion exchange column load (post trypsin digestion); Lane 3: Anion exchange column flow through; Lane 4-12: Anion exchange column elution fractions E1-E9.

FIG. 20 shows the complete purification process of a CCD-PCM-XTEN construct, as described in Example 8. FIG. 20A shows a SDS-PAGE analysis of fraction of CCD-PCM-XTEN after cation exchange capture step. The materials per lane are: Lane 1: Marker; Lane 2: Cation exchange column load; Lane 3-5: Cation exchange column flow through/wash fractions 1-3; Lane 6: Cation exchange elution; Lane 7: Cation exchange column strip. FIG. 20B shows SDS-PAGE analysis of anion exchange polishing step fractions. The materials per lane are: Lane 1: Marker; Lane 2: Anion exchange column load (post trypsin digestion); Lane 3: Anion exchange column flow through; Lane 4-17: Anion exchange column elution fractions E1-E14; Lane 18: Marker; Lane 19: Anion exchange column load (post trypsin digestion); Lane 20-33: Anion exchange column elution fractions E15-E24; Lane 34: anion exchange column strip.

FIG. 21 depicts results from the experiments to synthesize 3x-MMAE-CCD-XTEN and 3x-MMAE-CCD-PCM-XTEN. FIG. 21A is an analytical C4 RP-HPLC trace of 3x-MMAE-CCD-XTEN demonstrating >95% purity, as described in Example 11. FIG. 21B is an analytical C4 RP-HPLC trace of 3x-MMAE-CCD-PCM-XTEN demonstrating >95% purity, as described in Example 12.

FIG. 22 depicts results from the experiments to synthesize MCC-3x-MMAE-CCD-XTEN, as described in Example 15. FIG. 22A is an analytical C4 RP-HPLC trace of MCC-3x-MMAE-CCD-XTEN demonstrating >95% purity. FIG. 22B is a non-reducing SDS polyacrylamide gel with molecular weight markers (lane 1), MCC-3x-MMAE-CCD-XTEN (lane 2), and maleimide reactivity assessment of MCC-3x-MMAE-CCD-XTEN reaction with Cys-XTEN (lane 3).

FIG. 23 depicts results from the experiments to synthesize an aHER2-targeted CCD-XTEN-drug conjugate, as described in Example 18. FIG. 23A is a non-reducing SDS polyacrylamide gel with molecular weight markers (lane 1) and purified aHER2-targeted CCD-XTEN-drug conjugate from reaction of aHER2-XTEN and MCC-3x-MMAE-CCD-XTEN (lane 2). FIG. 23B is ESI-MS data demonstrating purity and intact mass of aHER2-targeted CCD-XTEN-drug conjugate. FIG. 23C is analytical SEC-HPLC data demonstrating monomeric purity of aHER2-targeted CCD-XTEN-drug conjugate.

FIG. 24 depicts results from the experiments to synthesize an aHER2-targeted CCD-XTEN-drug conjugate with PCM, as described in Example 19. FIG. 24A is a non-reducing SDS polyacrylamide gel with molecular weight markers (lane 1) and purified aHER2-targeted CCD-XTEN-drug conjugate with PCM from reaction of aHER2-XTEN and MCC-3x-MMAE-CCD-PCM-XTEN (lane 2). FIG. 24B is ESI-MS data demonstrating purity and intact mass of aHER2-targeted CCD-XTEN-drug conjugate. FIG. 24C is analytical SEC-HPLC data demonstrating monomeric purity of aHER2-targeted CCD-XTEN-drug conjugate with PCM.

FIG. 25 depicts results from the experiments to synthesize aHER2-targeted XTEN-3x-DM1 conjugate, as described in Example 16. FIG. 25A is a non-reducing SDS polyacrylamide gel with molecular weight markers (lane 1) and purified aHER2-targeted XTEN-3xDM1 conjugate (lane 2). FIG. 25B is ESI-MS data demonstrating purity and intact mass of aHER2-targeted XTEN-3xDM1. FIG. 25C is analytical SEC-HPLC data demonstrating monomeric purity of aHER2-targeted XTEN-3xDM1.

FIG. 26 shows an SDS-PAGE gels of samples from a stability study of XTEN_AE864. The XTEN_AE864 was incubated in rat plasma (FIG. 26A), rat kidney homogenate (FIG. 26B, left), and PBS buffer (FIG. 26B, right) for up to 7 days at 37° C., as described in Example 29. Samples were withdrawn at 0 hours, 4 hours, 24 hours, 7 days, and XTEN were extracted by methanol precipitation and analyzed by SDS-PAGE followed by staining with Stains-all. The location of the full-length XTEN864 is shown by the arrow.

FIG. 27 shows the near UV circular dichroism spectrum of Ex4-XTEN_AE864, performed as described in Example 30.

FIG. 28 is a schematic of the logic flow chart of the algorithm BlockScore (Example 32). In the figure the following legend applies: i, j—counters used in the control loops that run through the entire sequence; HitCount—this variable is a counter that keeps track of how many times a subsequence encounters an identical subsequence in a block; SubSeqX—this variable holds the subsequence that is being checked for redundancy; Sub SeqY—this variable holds the subsequence that the SubSeqX is checked against; BlockLen—this variable holds the user determined length of the block; SegLen—this variable holds the length of a segment. The program is hardcoded to generate scores for subsequences of lengths 3, 4, 5, 6, 7, 8, 9, and 10; Block—this variable holds a string of length BlockLen. The string is composed of letters from an input XTEN sequence and is determined by the position of the i counter; SubSeqList—this is a list that holds all of the generated subsequence scores.

FIG. 29 depicts results from the experiments to synthesize aHER2-targeted XTEN-3x-MMAE conjugate, as described in Example 17. FIG. 29A is a non-reducing SDS polyacrylamide gel with molecular weight markers (lane 1) and purified aHER2-targeted XTEN-3xMMAE conjugate (lane 2). FIG. 29B is ESI-MS data demonstrating purity and intact mass of aHER2-targeted XTEN-3xMMAE.

FIG. 30 depicts results from the experiments to synthesize folate-targeted CCD-XTEN-drug conjugate, as described in Example 20. FIG. 30A is an analytical C4 RP-HPLC trace of purified folate-targeted CCD-XTEN-drug conjugate from reaction of folate-AHHAC and MCC-3x-MMAE-CCD-XTEN. FIG. 30B is a non-reducing SDS polyacrylamide gel with molecular weight markers (lane 1) and purified folate-targeted CCD-XTEN-drug conjugate (lane 2).

FIG. 31 depicts results from the experiments to synthesize folate-targeted CCD-XTEN-drug conjugate with PCM, as described in Example 21. FIG. 31A is an analytical C4 RP-HPLC trace of purified folate-targeted CCD-XTEN-drug conjugate with PCM from reaction of folate-AHHAC and MCC-3x-MMAE-CCD-PCM-XTEN. FIG. 31B is a non-reducing SDS polyacrylamide gel with molecular weight markers (lane 1) and purified folate-targeted CCD-XTEN-drug conjugate with PCM (lane 2).

FIG. 32 depicts analytical C4 RP-HPLC result from the experiments to synthesize 3x-MMAE-XTEN, as described in Example 10.

FIG. 33 depicts results from the experiments to synthesize 3x-MMAE-CCD-XTEN, as described in Example 11, and 3x-MMAE-XTEN, as described in Example 10. FIG. 33A shows a scheme for the conjugation of drug payload to CCD-XTEN or cysteine-engineered XTEN. FIG. 33B depicts the analytical C4 RP-HPLC traces for drug conjugation to CCD-XTEN or cysteine-engineered XTEN.

FIG. 34 depicts different formats of Targeting Moiety-CCD-PCM-XTEN-Payload conjugates in which the PCM domain is optional. The attached XTEN helps to extend systemic half-life to the composition after administration to a subject. When the composition is in the tumor microenvironment, over-expressed proteases of the tumor cleave the PCM (if present), releasing the terminal XTEN, resulting in better penetration of the smaller remaining segment carrying targeting moiety fused to the CCD with linked Payload. FIG. 34A shows one or multiple Payload A molecules attached to the CCD that will remain together with the Targeting Moiety (TM1) after proteolytic cleavage at the PCM, releasing the XTEN from the composition. FIG. 34B shows one or multiple Payload A molecules attached to the CCD that will remain together with the Targeting Moiety (TM1) and XTEN, with no proteolytic cleavage of the XTEN away from the composition. FIG. 34C shows varying number of XTENs attached to PCM by multivalent cross-linkers capable of being released upon cleavage of the PCM, resulting in better penetration of the smaller N-terminal segment carrying the targeting moiety and the CCD with the linked Payloads. FIG. 34D shows that the length of XTENs released after cleavage of PCM can be varied in the targeted conjugate compositions, for purposes of adjusting pharmacokinetics, tumor penetration, and shielding of the payloads and targeting moieties, the latter property also illustrated in FIG. 35.

FIG. 35 illustrates different formats of the targeted conjugate composition contructs in which the Targeting Moiety (TM1) is shielded by protease-releasable XTEN(s) and/or steric hindrance of the compound configuration. In these exemplary configurations, TM1 is only exposed upon PCM cleavage in the tumor microenvironment and becomes accessible to its ligand. Conversely, normal tissues with high expression of the cancer target but otherwise lacking or having low expression of protease will be spared due to lack of protease over-expression seen in the tumor microenvironment. FIG. 35A shows a construct design linked to a single protease-cleavable XTEN. FIG. 35B shows a construct design linked to multiple protease-cleavable XTENs to achieve a better shielding effect.

FIG. 36 illustrates the chemical structure and sequences of a folate-targeted XTEN-conjugate with a folate targeting moiety conjugated to a CCD-XTEN (SEQ ID NO: 770) (FIG. 36A) or a folate targeting moiety conjugated to a CCD-PCM-XTEN (FIG. 36B) in which molecules of MMAE (SEQ ID NO: 771) (FIG. 36C) are conjugated to the Z modified cysteine residues of the CCD.

FIG. 37 shows the conjugation of multiple copies of PCM-TM1-CCD-Payload-PCM-XTEN2 construct or multiple copies of a PCM-TM1-CCD-Payload construct onto one single backbone XTEN. FIG. 37A shows XTEN1 containing three reactive groups (IB) FIG. 37B shows a fusion protein containing a reactive group (1A) on a PCM sequence fused to a targeting moiety (TM1) fused to a CCD segment carrying three copies of Payload A and an XTEN2; FIG. 37C shows a fusion protein containing a reactive group (1A) on a PCM sequence fused to a targeting moiety (TM1) fused to a CCD segment carrying three copies of Payload A. FIG. 37D shows the reaction product of the final conjugate of FIG. 37A and 37B with one XTEN backbone sequence carrying multiple copies of protease-releasable TM1-CCD-3x_Payload A-XTEN2 conjugate while FIG. 37E shows the reaction product of the final conjugate of FIG. 37A and 37C with one XTEN backbone sequence carrying multiple copies of protease-releasable TM1-CCD-3x_Payload A conjugate. The TM1 of the final conjugates are shielded but the construct is likely to infiltrate tumor tissue more than normal tissue due to the enhanced permeability and retention (EPR) effect imparted by the XTEN.

FIG. 38 shows a schematic example the cleavage, binding, and processing of an targeted conjugate composition comprising fusion proteins of targeting moieties fused to CCD and linked toxin payloads conjugated to an XTEN backbone by a protease cleavage moiety (PCM) that has a sequence capable of being cleaved by a protease in the microenvironment of the target cell, such as a tumor cell. When the conjugate is in the microenviroment of a tumor overexpressing protease capable of cleaving the PCM, the component of the cleaved conjugate with the TM1 (fused to CCD with linked cytotoxic Payload A) binds to the target receptor that is over-expressed on the cancer cell. Receptor binding results in internalization of the bound fusion protein conjugate followed by proteolytic break down and the intracellular liberation of Payload A, which is toxic to the cell.

FIG. 39 shows the conjugation of multiple copies of either reactive PCM-TM1-CCD-Payload-XTEN2 (FIG. 39B) or reactive PCM-TM1-CCD-Payload (FIG. 39C) molecules onto one single backbone XTEN. FIG. 39A depicts the backbone XTEN containing three reactive groups (IB), as well as a targeting moiety (TM2), which serves to bring the drug in the proximity of tumor tissue. FIG. 39D depicts the final conjugate construct with one TM2-targeted molecule carrying, in this case, three copies of protease-releasable TM1-CCD-3x_Payload A-XTEN2 conjugates. FIG. 39E depicts the final conjugate construct with one TM2-targeted molecule carrying, in this case, three copies of protease-releasable TM1-CCD-3x_Payload A conjugates.

FIG. 40 shows a schematic example of the cleavage, binding, and processing of a conjugate of FIG. 39, comprising targeting moieties and toxin payloads linked to a backbone XTEN by a protease cleavage moiety (PCM) that exerts selective action on a target cell, such as a tumor cell. In this case, a second targeting domain (TM2) on the XTEN backbone serves to bring the entire molecule to the proximity of tumor but does not internalize. This allows sufficient residence time in the tumor micro-environment for the tumor-expressed protease to act on the PCM, thus releasing and “activating” the TM1-CCD-Payload_A conjuate by the release from the shielding effect of the intact composition.

FIG. 41 shows a schematic of a mechanism of action for reducing and then restoring the potency of an active moiety in an XTEN-conjugate in a selective fashion. In blood and other normal, healthy tissues lacking (or with reduced) protease activity, the XTEN-conjugate of FIG. 41A remains mostly intact, maintaining long serum half-life and low affinity for normal tissue having receptors for the active moiety. In inflamed tissue, where protease levels are elevated, the protease would cleave the PCM (FIG. 41B), liberating the payload in the proximity of the inflammed tissue, thereby regaining potency and the ability to exert its pharmacologic action.

FIG. 42 shows the conjugation of one parental XTEN backbone (FIG. 42A) carrying multiple copies of protease-releasable active moiety (FIG. 42B) or active moiety-XTEN2 fusion proteins (FIG. 42C). In both cases, active moieties are blocked until over-expressed proteases in inflamed tissue liberate them, as described for FIG. 41.

FIG. 43 depicts different formats of protease-activatable antibody fragment. FIG. 43A depicts scFv oriented as a variable heavy chain linked to a variable light chain, or vice versa; FIG. 43B depicts protease-cleavable XTEN of various lengths fused to either or both termini of scFv. The affinity of scFv is impaired due to XTEN fusion and is restored upon protease cleavage in target tissue. FIG. 43C depicts insertion of protease-cleavable XTEN into non-essential CDRs, such as CDR2 and CDR3 of the variable light chain The inserted XTEN segment does not affect overall folding of the scFv but offers a shielding effect to other CDRs and thus impedes scFv-XTEN fusion binding to its target until protease cleavage. FIG. 43D depicts various permutations of terminus-fusion and CDR insertions of protease-cleavable XTEN to scFv.

FIG. 44 shows the results of an assay to determine the action of an MMP-9 enzyme on a peptidyl cleavage moiety. 10 μM of XTEN864-His with the PLGLAG cleavage site (SEQ ID NO: 4) was incubated with 0.1 ng/μL of MMP-9 in 20 uL reactions. Reactions were incubated at 37° C. for up to one hour, with aliquots collected at 10 minute intervals by stopping digestion with the addition of EDTA to 20 mM. Analysis of the samples to determine percentage of cleaved product was performed by C18 RP-HPLC (FIG. 44A). Two negative controls were also included in the assay: one to confirm that digestion did not occur in the absence of MMP-9, and one to confirm that digestion did not occur in the presence of APMA alone, the chemical utilized in zymogen activation (FIG. 44B).

FIG. 45 shows the results of the proteolytic cleavage assay of an XTEN comprising a proteolytic cleavage moiety BSRS-1, as described in Example 9. FIG. 45A is the results from an SDS-PAGE assay of BSRS1-XTEN digested with MTSP-1, uPA, MMP-2, MMP-7 where the digested products run at a smaller apparent molecular weight compared to the uncleaved starting material. FIG. 45B shows results of an RPC18 HPLC analysis of the pre- and post-digestion samples, with a clear shift in retention time.

FIG. 46 depicts configurations of conjugate compositions wherein the TM1 is linked to the composition either recombinantly or is conjugated to the fusion protein. FIG. 46A depicts the configuration of a conjugate composition comprising a fusion protein comprising an XTEN, a peptidyl cleavage moiety (PCM), a CCD, and the TM1 at the N-terminus, wherein the TM1, the CCD, the PCM and the XTEN are all linked as a recombinant polypeptide, and the three identical molecules of thePayload A are conjugated to the fusion protein at cysteine residues of the CCD. FIG. 46B depicts a composition that has the same components as FIG. 46A, but the N- to C-terminus orientation of the components is reversed. FIG. 46C depicts the configuration of a conjugate composition comprising a fusion protein comprising an XTEN, a peptidyl cleavage moiety (PCM), a CCD, and the TM1 is conjugated to the N-terminus of the CCD-PCM-XTEN fusion protein (the arrow indicates the site of conjugation).

FIG. 47 depicts configurations of conjugate compositions wherein the TM1 is linked to the composition either recombinantly or is conjugated to the fusion protein. FIG. 47A depicts the configuration of a conjugate composition comprising an XTENconjugated to a fusion protein of a peptidyl cleavage moiety (PCM), a targeting moiety (TM1), and a CCD. Three molecules of Payload A are conjugated to the fusion protein at cysteine residues of the CCD. FIG. 47B depicts the configuration of a conjugate composition comprising an XTEN selected from the group consisting of the sequences of Table 10 conjugated to a peptidyl cleavage moiety (PCM), wherein the PCM sequence is selected from the group consisting of the PCM sequences of Table 7 linked to a targeting moiety (TM1), which is conjugated to a CCD. Three molecules of Payload A are conjugated to the cysteine residues of the CCD. The arrows indicate the sites of conjugation.

FIG. 48 depicts configurations of targeted conjugate compositions wherein the TM1 is linked to the composition either recombinantly or is conjugated to the fusion protein. FIG. 48A depicts the configuration of a conjugate composition comprising two identical molecules of an XTEN linked to a trimeric cross-linker, a fusion protein comprising i) a peptidyl cleavage moiety (PCM); ii) a targeting moiety (TM1) that is recombinantly linked between the PCM and the CCD; and iii) a CCD with three molecules of a Payload A conjugated to the fusion protein at cysteine residues of the CCD. The arrows indicate the sites of conjugation. FIG. 48B depicts the same general configuration as FIG. 48A but the TM1 is recombinantly linked to the PCM and is conjugated to the CCD.

FIG. 49 depicts configurations of targeted conjugate compositions wherein the TM1 is linked to the composition either recombinantly or is conjugated to the fusion protein. FIG. 49A depicts the configuration of a conjugate composition comprising an XTEN backbone; three identical molecules of a fusion protein comprising i) a peptidyl cleavage moiety (PCM); ii) a targeting moiety (TM1) that is recombinantly linked to the PCM and the CCD in each of the three fusion proteins; iii) a CCD; and nine molecules of Payload A wherein three molecules each Payload A are conjugated to each of the three fusion proteins at cysteine residues of the CCD. The arrows indicate the sites of conjugation. FIG. 49B depicts the same general configuration as FIG. 49A but the TM1 is recombinantly linked to the PCM and is conjugated to the CCD bearing the Payload A molecules.

FIG. 50 depicts configurations of targeted conjugate compositions. FIG. 50A depicts the configuration of a conjugate composition comprising (a) a first fusion protein comprising a backbone XTEN and a targeting moiety (TM2) that is recombinantly linked to the PCM and the CCD bearing the three Payload A molecules; (b) three identical molecules of a fusion protein conjugated to cysteine residues of the XTEN comprising i) a peptidyl cleavage moiety (PCM); ii) a targeting moiety (TM1) wherein the TM1 binds a different target than the TM2; iii) a CCD; and (c) nine molecules of Payload A wherein three molecules each Payload A are conjugated to each of the three fusion proteins at cysteine residues of the CCD. The arrows indicate the sites of conjugation. FIG. 50B depicts the same general configuration as FIG. 50A but the TM1 is recombinantly linked to the PCM and is conjugated to the CCD bearing the Payload A molecules.

FIG. 51 depicts the configuration of a conjugate composition comprising an immunoglobulin molecule and two molecules of a cleavable conjugate composition comprising a fusion protein comprising i) an XTEN; ii) a peptidyl cleavage moiety (PCM); iii) a CCD; and iv) three identical molecules of Payload A wherein the three molecules each Payload A are conjugated to each of the fusion proteins at cysteine residues of the CCD. The arrows indicate the sites of conjugation of the fusion protein to the immunoglobulin.

FIG. 52 depicts the conjugate compositions of FIG. 46 reacted with a protease capable of cleaving the PCM (indicated by the scissors) and the resulting reaction products. FIG. 52A (a recombinant attachment of the TM1) and FIG. 52B (a conjugate attachment of the TM1) both depict the location of the cleavage at the PCM and the release of the bulky XTEN from the remainder of the composition, with the remainder, which is of greatly reduced molecular size and is freed from the shielding effect of the XTEN, able to bind to and deliver the payload drugs to the target tissue.

FIG. 53 depicts depicts the conjugate compositions of FIG. 47 reacted with a protease capable of cleaving the PCM (indicated by the scissors) and the reaction products. FIG. 53A (a recombinant attachment of the TM1) and FIG. 53B (a conjugate attachment of the TM1) both depict the location of the cleavage at the PCM and the release of the bulky XTEN from the remainder of the composition, with the remainder, which is of greatly reduced molecular size and is freed from the shielding effect of the XTEN, able to bind to and deliver the payload drugs to the target tissue.

FIG. 54 depicts depicts the conjugate compositions of FIG. 48 reacted with a protease capable of cleaving the PCM (indicated by the scissors) and the resulting reaction products. FIG. 54A (a recombinant attachment of the TM1) and FIG. 54B (a conjugate attachment of the TM1) both depict the location of the cleavage at the PCM and the release of the bulky XTEN from the remainder of the composition, with the remainder, which is of greatly reduced molecular size and is freed from the shielding effect of the XTEN, able to bind to and deliver the payload drugs to the target tissue.

FIG. 55 depicts depicts the conjugate compositions of FIG. 49 reacted with a protease capable of cleaving the PCM (indicated by the scissors) and the resulting reaction products. FIG. 55A (a recombinant attachment of the TM1) and FIG. 55B (a conjugate attachment of the TM1) both depict the location of the cleavage at the PCM and the release of the bulky dimer of two molecules of XTEN (linked to each other) from the remainder of the composition, with the remainder, which is of greatly reduced molecular size and is freed from the shielding effect of the XTEN, able to bind to and deliver the payload drugs to the target tissue.

FIG. 56 depicts the conjugate compositions of FIG. 50 reacted with a protease capable of cleaving the PCM (indicated by the scissors) and the resulting reaction products. FIG. 56A (a recombinant attachment of the TM1) and FIG. 56B (a conjugate attachment of the TM1) both depict the location of the cleavage at the PCM and the release of the bulky XTEN from the remainder of the composition, with the remainder, the three molecules of a TM1 linked to a CCD with 3 molecules of payload drug linked to each CCD, able to bind to and deliver the payload drugs to the target tissue.

FIG. 57 depicts the conjugate compositions of FIG. 51 reacted with a protease capable of cleaving the PCM (indicated by the scissors) and the resulting reaction products.

FIG. 58 depicts results from the experiment to determine the in vitro activity of FA-XTEN432-3xMMAF and XTEN432-3xMMAF in KB cells, as described in Example 35.

FIG. 59 depicts results from the experiment to determine the in vitro activity of FA-XTEN864-3xMMAF and XTEN864-3xMMAF in KB cells, as described in Example 61.

FIG. 60 depicts results from the experiment to determine the PK of FA-XTEN432-3xMMAF and XTEN432-3xMMAF in nu/nu mice, as described in Example 36.

FIG. 61 depicts results from the experiment to determine the MTD of FA-XTEN432-3xMMAF in nu/nu mice, as described in Example 36.

FIG. 62 depicts results from the experiment to determine the efficacy of FA-XTEN432-3xMMAF and XTEN432-3xMMAF in a KB xenograft mouse model, as described in Example 36.

FIG. 63 depicts results from the experiment to determine the safety of FA-XTEN432-3xMMAF and XTEN432-3xMMAF in a KB xenograft mouse model, as described in Example 36.

FIG. 64 depicts results from the experiment to determine the efficacy of FA-XTEN864-3xMMAF and XTEN864-3xMMAF in a KB xenograft mouse model, as described in Example 36.

FIG. 65 depicts results from the experiment to determine the safety and tolerability of FA-XTEN864-3xMMAF and XTEN864-3xMMAF in a KB xenograft mouse model, as described in Example 36.

FIG. 66 depicts the configuration of a conjugate cleavable composition comprising an XTEN; three different peptidyl cleavage moieties (PCM1, PCM2, PCM3; collectively represented by BSRS1) integrated into the TM1-XTEN-PCM-CCD fusion protein sequence wherein each PCM sequence is a different sequence; a CCD; and molecules of a targeting moiety (TM1) fused to the XTEN; and three molecules of Payload A wherein the Payload A are conjugated to cysteine residues of the CCD. The figure schematically demonstrates that the composition is capable of being cleaved by three different proteases wherein the cleavage by any one protease results in a different reaction product but all result in the release of the bulky XTEN from the composition.

FIG. 67 shows the plasma concentrations of the indicated treatment groups of four different constructs dosed at 26 nmol/kg, as described in Example 37.

FIG. 68 shows the plasma concentrations of the treatment groups of the same four constructs as per FIG. 67, dosed at 460 nmol/kg, as described in Example 37.

FIG. 69 shows the tissue concentrations of the two indicated treatment groups dosed at 26 nmol/kg, with FIG. 69A showing results at 24 h and FIG. 69B showing results at 72 h, as described in Example 37.

FIG. 70 shows the tissue concentrations of the two indicated treatment groups dosed at 460 nmol/kg, with FIG. 70A showing results at 24 h and FIG. 70B showing results at 72 h, as described in Example 37.

FIG. 71 shows the tissue concentrations of the two indicated treatment groups dosed at 26 nmol/kg, with FIG. 71A showing results at 24 h and FIG. 71B showing results at 72 h, as described in Example 37.

FIG. 72 shows the tissue concentrations of the two indicated treatment groups dosed at 460 nmol/kg, with FIG. 72A showing results at 24 h and FIG. 72B showing results at 72 h, as described in Example 37.

FIG. 73 shows the tumor and the plasma concentrations of the indicated two targeted constructs at 24 and 72 h intervals, as described in Example 37.

FIG. 74 shows the tumor volume data over time for the three indicated treatment groups and control, as described in Example 38.

FIG. 75 shows the body weight data over time for the three indicated treatment groups and control, as described in Example 38.

FIG. 76 shows the plasma concentrations of the five treatment groups dosed at 2 mg/kg each, as described in Example 39.

FIG. 77 shows the plasma concentrations of the five treatment groups dosed at 2 mg/kg each, as described in Example 39.

FIG. 78 shows binding of the two targeted constructs for its ligand, as described in Example 40.

FIG. 79 depicts results from the experiments to determine the in vitro selective activity of FA-XTEN432-3xMMAF in the presence and absence of folic acid; with FIG. 79A showing results for JEG-3, FIG. 79B for SW620 and FIG. 79C for SK-BR-3, as described in Example 41.

FIG. 80 shows the tumor volume data over time for the two treatment groups, as described in Example 42.

FIG. 81 shows the body weight data over time for the two treatment groups, as described in Example 42.

FIG. 82 shows the tumor volume data over time for the three treatment groups, as described in Example 42.

FIG. 83 depicts results from the experiments to determine the in vitro activity of CXTEN432-3xMMAE and anti-HER2scFv-3xMMAE-CXTEN720 in the presence and absence of trastuzumab; with FIG. 83A showing results for SK-BR-3, FIG. 83B for BT474, FIG. 83C for HCC1954, FIG. 83D for NCI-N87 and FIG. 83E for SK-OV-3, as described in Example 44.

FIG. 84 depicts results from the experiments to determine the in vitro activity of CXTEN432-3xDM1 and anti-HER2scFv-3xDM1-CXTEN720 in the presence and absence of trastuzumab; with FIG. 84A showing results for SK-BR-3, FIG. 84B for BT474, FIG. 84C for HCC1954, FIG. 84D for NCI-N87 and FIG. 84E for SK-OV-3, as described in Example 44.

FIG. 85 depicts results from the experiments to determine the in vitro activity of anti-HER2scFv-3xMMAE-CCD-XTEN757, protease treated and untreated anti-HER2scFv-3xMMAE-CCD-BSRS1-XTEN753 in NCI-N87, as described in Example 45.

FIG. 86 shows neutrophil elastase (NE) digestion of XTEN as described in Example 48. The materials per lane are: Lane 1: Marker; Lane 2: Undigested XTEN_AE864; Lane 3: XTEN_AE864 incubated at 37° C. with NE at 1:1000 molar ratio for 2 hours; Lane 4: XTEN_AE864 incubated at 37° C. with NE at 1:100 molar ratio for 2 hours.

FIG. 87 shows schematic representations of scFv and concatenate configurations. FIG. 87A shows two configurations of scFv that have, in a N-terminus to C-terminus orientations, VL-linker-VH or VL-linker-VH components of the framework or CDR variable segments depicted. FIG. 87B shows two configurations of concatenate fusion proteins that have, in a N-terminus to C-terminus orientations, FRL4 or FRH4 segments fused to CCD, PCM, and an XTEN sequence. FIG. 87C shows two configurations of concatenate fusion proteins that have, in a N-terminus to C-terminus orientations, an XTEN sequence fused to PCM, CCD, and FRL1 or FRH1 segments.

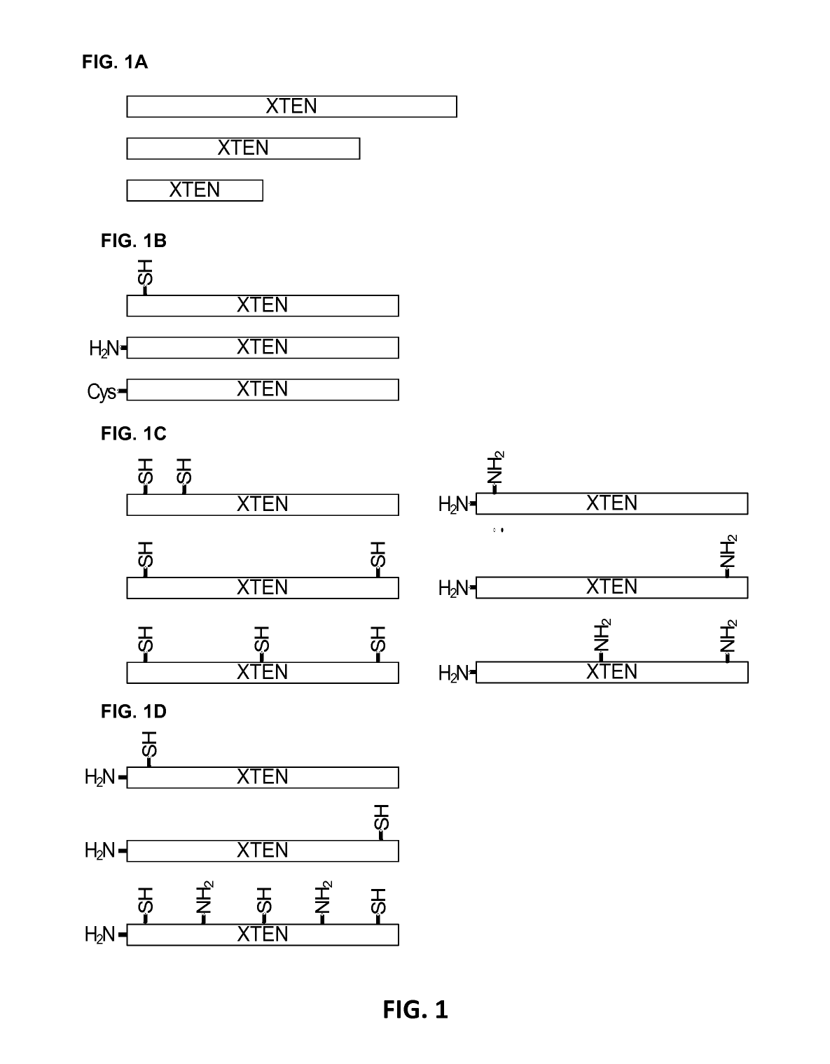

### DETAILED DESCRIPTION OF THE INVENTION

Before the embodiments of the invention are described, it is to be understood that such embodiments are provided by way of example only, and that various alternatives to the embodiments of the invention described herein may be employed in practicing the invention. Numerous variations, changes, and substitutions will now occur to those skilled in the art without departing from the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### DEFINITIONS

In the context of the present application, the following terms have the meanings ascribed to them unless specified otherwise:

As used throughout the specification and claims, the terms “a”, “an” and “the” are used in the sense that they mean “at least one”, “at least a first”, “one or more” or “a plurality” of the referenced components or steps, except in instances wherein an upper limit is thereafter specifically stated. Therefore, a “payload”, as used herein, means “at least a first payload” but includes a plurality of payloads. The operable limits and parameters of combinations, as with the amounts of any single agent, will be known to those of ordinary skill in the art in light of the present disclosure.

The terms “polypeptide”, “peptide”, and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

As used herein, the term “amino acid” refers to either natural and/or unnatural or synthetic amino acids, including but not limited to both the D or L optical isomers, and amino acid analogs and peptidomimetics. Standard single or three letter codes are used to designate amino acids.

A “pharmacologically active” agent includes any drug, compound, composition of matter or mixture desired to be delivered to a subject, e.g. therapeutic agents, diagnostic agents, or drug delivery agents, which provides or is expected to provide some pharmacologic, often beneficial, effect that can be demonstrated in vivo or in vitro. Such agents may include peptides, proteins, carbohydrates, nucleic acids, nucleosides, oligonucleotides, and small molecule synthetic compounds, or analogs thereof.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine (S), and threonine (T).

The term “non-naturally occurring,” as applied to sequences and as used herein, means polypeptide or polynucleotide sequences that do not have a counterpart to, are not complementary to, or do not have a high degree of homology with a wild-type or naturally-occurring sequence found in a mammal. For example, a non-naturally occurring polypeptide or fragment may share no more than 99%, 98%, 95%, 90%, 80%, 70%, 60%, 50% or even less amino acid sequence identity as compared to a natural sequence when suitably aligned.

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. A number of scales have been developed. An example is a scale developed by Levitt, M, et al., J Mol Biol (1976) 104:59, which is listed in Hopp, T P, et al., Proc Natl Acad Sci USA (1981) 78:3824. Examples of “hydrophilic amino acids” are arginine, lysine, threonine, alanine, asparagine, and glutamine, aspartate, glutamate, serine, and glycine. Examples of “hydrophobic amino acids” are tryptophan, tyrosine, phenylalanine, methionine, leucine, isoleucine, and valine.

A “fragment” when applied to a biologically active protein, is a truncated form of a the biologically active protein that retains at least a portion of the therapeutic and/or biological activity. A “variant,” when applied to a biologically active protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein. As used herein, the term “biologically active protein variant” includes proteins modified deliberately, as for example, by site directed mutagenesis, synthesis of the encoding gene, insertions, or accidentally through mutations and that retain activity.

The term “sequence variant” means polypeptides that have been modified compared to their native or original sequence by one or more amino acid insertions, deletions, or substitutions. Insertions may be located at either or both termini of the protein, and/or may be positioned within internal regions of the amino acid sequence. A non-limiting example is insertion of an XTEN sequence within the sequence of the biologically-active payload protein. Another non-limiting example is substitution of an amino acid in an XTEN with a different amino acid. In deletion variants, one or more amino acid residues in a polypeptide as described herein are removed. Deletion variants, therefore, include all fragments of a payload polypeptide sequence. In substitution variants, one or more amino acid residues of a polypeptide are removed and replaced with alternative residues. In one aspect, the substitutions are conservative in nature.

The term “moiety” means a component of a larger composition or that is intended to be incorporated into a larger composition, such as a functional group of a drug molecule or a targeting peptide joined to a larger polypeptide.

As used herein, “terminal XTEN” refers to XTEN sequences that have been fused to or in the N- or C-terminus of the payload when the payload is a peptide or polypeptide.

The term “peptidyl cleavage moiety” or “PCM” refers to a cleavage sequence in cleavable conjugate compositions that can be recognized and cleaved by one or more proteases, effecting release of a payload, an XTEN, or a portion of an XTEN-conjugate from the XTEN-conjugate. As used herein, “mammalian protease” means a protease that normally exists in the body fluids, cells or tissues of a mammal. PCM sequences can be engineered to be cleaved by various mammalian proteases that are present in or proximal to target tissues in a subject or mammalian cell lines in an in vitro assay. Other equivalent proteases (endogenous or exogenous) that are capable of recognizing a defined cleavage site can be utilized. It is specifically contemplated that the PCM sequence can be adjusted and tailored to the protease utilized and can incorporate linker amino acids to join to adjacent polypeptides

The term “within”, when referring to a first polypeptide being linked to a second polypeptide, encompasses linking that connects the N-terminus of the first or second polypeptide to the C-terminus of the second or first polypeptide, respectively, as well as insertion of the first polypeptide into the sequence of the second polypeptide. For example, when an XTEN is linked “within” a payload polypeptide, the XTEN may be linked to the N-terminus, the C-terminus, or may be inserted between any two amino acids of the payload polypeptide.

“Activity” as applied to the subject compositions provided herein, refers to an action or effect, including but not limited to receptor binding, antagonist activity, agonist activity, a cellular or physiologic response, or an effect generally known in the art for the payload component of the composition, whether measured by an in vitro, ex vivo or in vivo assay or a clinical effect.

As used herein, the term “ELISA” refers to an enzyme-linked immunosorbent assay as described herein or as otherwise known in the art.

A “host cell” includes an individual cell or cell culture which can be or has been a recipient for the subject vectors such as those described herein. In some cases the host cell is a prokaryote, which may include E. coli. In other cases, a host cell is a eukaryotic cell, which may be a yeast, a non-human mammalian cell, or a human-derived cell. Host cells include progeny of a single host cell. The progeny may not necessarily be completely identical (in morphology or in genomic of total DNA complement) to the original parent cell due to natural, accidental, or deliberate mutation. A host cell includes cells transfected in vivo with a vector of this invention.

“Isolated” when used to describe the various polypeptides disclosed herein, means a polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is generally greater than that of its naturally occurring counterpart. In general, a polypeptide made by recombinant means and expressed in a host cell is considered to be “isolated.”

An “isolated” nucleic acid is a nucleic acid molecule that is identified and separated from at least one contaminant nucleic acid molecule with which it is ordinarily associated in the natural source of the polypeptide-encoding nucleic acid. For example, an isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal or extra-chromosomal location different from that of natural cells.

A “chimeric” protein contains at least one fusion polypeptide comprising at least one region in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Fused,” and “fusion” are used interchangeably herein, and refers to the joining together of two or more peptide or polypeptide sequences by recombinant means. A “fusion protein” or “chimeric protein” comprises a first amino acid sequence linked to a second amino acid sequence with which it is not naturally linked in nature.

“Operably linked” means that the DNA sequences being linked are contiguous, and in reading phase or in-frame. An “in-frame fusion” refers to the joining of two or more open reading frames (ORFs) to form a continuous longer ORF, in a manner that maintains the correct reading frame of the original ORFs. For example, a promoter or enhancer is operably linked to a coding sequence for a polypeptide if it affects the transcription of the polypeptide sequence. Thus, the resulting recombinant fusion protein is a single protein containing two or more segments that correspond to polypeptides encoded by the original ORFs (which segments are not normally so joined in nature).

“Crosslinking,” “conjugating,” “link,” “linking” and “joined to” are used interchangeably herein, and refer to the covalent joining of two different molecules by a chemical reaction. The crosslinking can occur in one or more chemical reactions, as described more fully, below.

The term “conjugation partner” as used herein, refers to the individual components that can be linked or are linked in a conjugation reaction.

The term “conjugate” is intended to refer to the heterogeneous molecule formed as a result of covalent linking of conjugation partners one to another, e.g., a biologically active payload covalently linked to a XTEN molecule or a cross-linker covalently linked to a reactive XTEN.

“Cross-linker” and “linker” and “cross-linking agent” are used interchangably and in their broadest context to mean a chemical entity used to covalently join two or more entities. For example, a cross-linker joins two, three, four or more XTEN, or joins a payload to an XTEN, as the entities are defined herein. A cross-linker includes, but is not limited to, the reaction product of small molecule zero-length, homo- or hetero-bifunctional, and multifunctional cross-linker compounds, the reaction product of two click-chemstry reactants. It will be understood by one of skill in the art that a cross-linker can refer to the covalently-bound reaction product remaining after the crosslinking of the reactants. The cross-linker can also comprise one or more reactants which have not yet reacted but which are capable to react with another entity.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Heterologous” means derived from a genotypically distinct entity from the rest of the entity to which it is being compared. For example, a glycine rich sequence removed from its native coding sequence and operatively linked to a coding sequence other than the native sequence is a heterologous glycine rich sequence. The term “heterologous” as applied to a polynucleotide or a polypeptide, means that the polynucleotide or polypeptide is derived from a genotypically distinct entity from that of the rest of the entity to which it is being compared.

The terms “polynucleotides”, “nucleic acids”, “nucleotides” and “oligonucleotides” are used interchangeably. They refer to nucleotides of any length, encompassing a singular nucleic acid as well as plural nucleic acids, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of recombination steps which may include cloning, restriction and/or ligation steps, and other procedures that result in expression of a recombinant protein in a host cell.

The terms “gene” and “gene fragment” are used interchangeably herein. They refer to a polynucleotide containing at least one open reading frame that is capable of encoding a particular protein after being transcribed and translated. A gene or gene fragment may be genomic or cDNA, as long as the polynucleotide contains at least one open reading frame, which may cover the entire coding region or a segment thereof. A “fusion gene” is a gene composed of at least two heterologous polynucleotides that are linked together.

As used herein, a “coding region” or “coding sequence” is a portion of polynucleotide which consists of codons translatable into amino acids. Although a “stop codon” (TAG, TGA, or TAA) is typically not translated into an amino acid, it may be considered to be part of a coding region, but any flanking sequences, for example promoters, ribosome binding sites, transcriptional terminators, introns, and the like, are not part of a coding region. The boundaries of a coding region are typically determined by a start codon at the 5′ terminus, encoding the amino terminus of the resultant polypeptide, and a translation stop codon at the 3′ terminus, encoding the carboxyl terminus of the resulting polypeptide. Two or more coding regions of the present invention can be present in a single polynucleotide construct, e.g., on a single vector, or in separate polynucleotide constructs, e.g., on separate (different) vectors. It follows, then, that a single vector can contain just a single coding region, or comprise two or more coding regions, e.g., a single vector can separately encode a binding domain-A and a binding domain-B as described below. In addition, a vector, polynucleotide, or nucleic acid of the invention can encode heterologous coding regions, either fused or unfused to a nucleic acid encoding a binding domain of the invention. Heterologous coding regions include without limitation specialized elements or motifs, such as a secretory signal peptide or a heterologous functional domain.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

“Homology” or “homologous” or “sequence identity” refers to sequence similarity or interchangeability between two or more polynucleotide sequences or between two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity compared to those sequences. Polypeptides that are homologous preferably have sequence identities that are at least 70%, preferably at least 80%, even more preferably at least 90%, even more preferably at least 95-99% identical.

“Ligation” as applied to polynucleic acids refers to the process of forming phosphodiester bonds between two nucleic acid fragments or genes, linking them together. To ligate the DNA fragments or genes together, the ends of the DNA must be compatible with each other. In some cases, the ends will be directly compatible after endonuclease digestion. However, it may be necessary to first convert the staggered ends commonly produced after endonuclease digestion to blunt ends to make them compatible for ligation.

The terms “stringent conditions” or “stringent hybridization conditions” includes reference to conditions under which a polynucleotide will hybridize to its target sequence, to a detectably greater degree than other sequences (e.g., at least 2-fold over background). Generally, stringency of hybridization is expressed, in part, with reference to the temperature and salt concentration under which the wash step is carried out. Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60° C. to 65° C. Alternatively, temperatures of about 65° C., 60° C., 55° C., or 42° C. may be used. SSC concentration may be varied from about 0.1 to 2×SSC, with SDS being present at about 0.1%. Such wash temperatures are typically selected to be about 5° C. to 20° C. lower than the thermal melting point for the specific sequence at a defined ionic strength and pH. The Tm is the temperature (under defined ionic strength and pH) at which 50% of the target sequence hybridizes to a perfectly matched probe. An equation for calculating Tm and conditions for nucleic acid hybridization are well known and can be found in Sambrook, J. et al., “Molecular Cloning: A Laboratory Manual,” 3^{rd} edition, Cold Spring Harbor Laboratory Press, 2001. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art.

The terms “percent identity,” percentage of sequence identity,” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured. The percentage of sequence identity is calculated by comparing two optimally aligned sequences over the window of comparison, determining the number of matched positions (at which identical residues occur in both polypeptide sequences), dividing the number of matched positions by the total number of positions in the window of comparison (i.e., the window size), and multiplying the result by 100 to yield the percentage of sequence identity. When sequences of different length are to be compared, the shortest sequence defines the length of the window of comparison. Conservative substitutions are not considered when calculating sequence identity.

“Percent identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity, thereby resulting in optimal alignment. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve optimal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Repetitiveness” used in the context of polynucleotide sequences refers to the degree of internal homology in the sequence such as, for example, the frequency of identical nucleotide sequences of a given length. Repetitiveness can, for example, be measured by analyzing the frequency of identical sequences.

In general, a marker (e.g. a protease or a ligand targeted by a TM) may be considered “associated with” or “colocalized with”a target cell or target tissue if it occurs with greater frequency or at higher concentration in, on, or in proximity to the target cell or target tissue, as compared to non-target cells or non-target tissue. For example, a marker may be considered associated with a target tissue if it occurs at a higher concentration in a fluid surrounding a target tissue than if found in fluid more distant from the target tissue. In some embodiments, a marker associated with a target cell is expressed by the target cell at a higher level than by non-target cells. In some embodiments, a marker associated with a target tissue is expressed at a higher level by one or more cells in the target tissue than by cells in non-target tissues. However, markers need not be expressed by a target cell or target tissue in order to be associated with such cell or tissue. For example, an inflammatory marker may be associated with a particular inflamed tissue but be expressed by an immune cell recruited to the tissue. Similarly, a microbial antigen that occurs with greater frequency in infected tissue is considered associated with such infected tissue, even though derived from the microbe. In some embodiments, a marker is associated with a disease or condition, such that the marker occurs more frequently or at higher levels among individuals with the disease or condition than in individuals without the disease or condition.

The term “expression” as used herein refers to a process by which a polynucleotide produces a gene product, for example, an RNA or a polypeptide. It includes without limitation transcription of the polynucleotide into messenger RNA (mRNA), transfer RNA (tRNA), small hairpin RNA (shRNA), small interfering RNA (siRNA) or any other RNA product, and the translation of an mRNA into a polypeptide. Expression produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation or splicing, or polypeptides with post translational modifications, e.g., methylation, glycosylation, the addition of lipids, association with other protein subunits, or proteolytic cleavage.

A “vector” or “expression vector” are used interchangably and refers to a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

“Serum degradation resistance,” as applied to a polypeptide, refers to the ability of the polypeptides to withstand degradation in blood or components thereof, which typically involves proteases in the serum or plasma. The serum degradation resistance can be measured by combining the protein with human (or mouse, rat, monkey, as appropriate) serum or plasma, typically for a range of days (e.g. 0.25, 0.5, 1, 2, 4, 8, 16 days), typically at about 37° C. The samples for these time points can be run on a Western blot assay and the protein is detected with an antibody. The antibody can be to a tag in the protein. If the protein shows a single band on the western, where the protein's size is identical to that of the injected protein, then no degradation has occurred. In this exemplary method, the time point where 50% of the protein is degraded, as judged by Western blots or equivalent techniques, is the serum degradation half-life or “serum half-life” of the protein.

The terms “t_{1/2}”, “half-life”, “terminal half-life”, “elimination half-life” and “circulating half-life” are used interchangeably herein and, as used herein means the terminal half-life calculated as ln(2)/Kₑₗ. Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. When a clearance curve of a given polypeptide is constructed as a function of time, the curve is usually biphasic with a rapid a-phase and longer β-phase. The typical β phase half-life of a human antibody in humans is 21 days.

“Active clearance” means the mechanisms by which a protein is removed from the circulation other than by filtration, and which includes removal from the circulation mediated by cells, receptors, metabolism, or degradation of the protein.

“Apparent molecular weight factor” and “apparent molecular weight” are related terms referring to a measure of the relative increase or decrease in apparent molecular weight exhibited by a particular amino acid or polypeptide sequence. The apparent molecular weight is determined using size exclusion chromatography (SEC) or similar methods by comparing to globular protein standards, and is measured in “apparent kD” units. The apparent molecular weight factor is the ratio between the apparent molecular weight and the actual molecular weight; the latter predicted by adding, based on amino acid composition, the calculated molecular weight of each type of amino acid in the composition or by estimation from comparison to molecular weight standards in an SDS electrophoresis gel. Determination of both the apparent molecular weight and apparent molecular weight factor for representative proteins is described in the Examples.

The terms “hydrodynamic radius” or “Stokes radius” is the effective radius (Rₕ in nm) of a molecule in a solution measured by assuming that it is a body moving through the solution and resisted by the solution's viscosity. In the embodiments of the invention, the hydrodynamic radius measurements of the XTEN polypeptides correlate with the “apparent molecular weight factor” which is a more intuitive measure. The “hydrodynamic radius” of a protein affects its rate of diffusion in aqueous solution as well as its ability to migrate in gels of macromolecules. The hydrodynamic radius of a protein is determined by its molecular weight as well as by its structure, including shape and compactness. Methods for determining the hydrodynamic radius are well known in the art, such as by the use of size exclusion chromatography (SEC), as described in U.S. Pat. Nos. 6,406,632 and 7,294,513. Most proteins have globular structure, which is the most compact three-dimensional structure a protein can have with the smallest hydrodynamic radius. Some proteins adopt a random and open, unstructured, or ‘linear’ conformation and as a result have a much larger hydrodynamic radius compared to typical globular proteins of similar molecular weight.

“Physiological conditions” refers to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers are listed in Sambrook et al. (2001). Physiologically relevant temperature ranges from about 25° C. to about 38° C., and preferably from about 35° C. to about 37° C.

A “single atom residue of a payload” means the atom of a payload that is chemically linked to XTEN after reaction with the subject XTEN or XTEN-linker compositions; typically a sulfur, an oxygen, a nitrogen, or a carbon atom. For example, an atom residue of a payload could be a sulfur residue of a cysteine thiol reactive group in a payload, a nitrogen molecule of an amino reactive group of a peptide or polypeptide or small molecule payload, a carbon or oxygen residue or a reactive carboxyl or aldehyde group of a peptide, protein or a small molecule or synthetic, organic drug.

A “reactive group” is a chemical structure that can be coupled to a second reactive group. Examples of reactive groups are amino groups, carboxyl groups, sulfhydryl groups, hydroxyl groups, aldehyde groups, azide groups. Some reactive groups can be activated to facilitate conjugation with a second reactive group, either directly or through a cross-linker. As used herein, a reactive group can be a part of an XTEN, a cross-linker, an azide/alkyne click-chemistry reactant, or a payload so long as it has the ability to participate in a chemical reaction. Once reacted, a conjugation bond links the residues of the payload or cross-linker or XTEN reactants.

“Controlled release agent”, “slow release agent”, “depot formulation” and “sustained release agent” are used interchangeably to refer to an agent capable of extending the duration of release of a polypeptide of the invention relative to the duration of release when the polypeptide is administered in the absence of agent. Different embodiments of the present invention may have different release rates, resulting in different therapeutic amounts.

The term “payload” as used herein refers to any protein, peptide sequence, small molecule, drug or composition of matter that has a biological, pharmacological or therapeutic activity or beneficial effect that can be demonstrated in an in vitro assay or when administered to a subject. Payload also includes a molecule that can be used for imaging or in vivo diagnostic purposes. Examples of payloads include, but are not limited to, cytokines, enzymes, hormones, blood coagulation factors, and growth factors, chemotherapeutic agents, antiviral compounds, toxins, anti-cancer drugs, cytotoxic drugs, radioactive compounds, and contrast agents, but, in the context of some aspects of the instant invention, specifically excludes targeting moieties, antibodies, antibody fragments, or organic small molecule compounds used solely to bind to receptors or ligands for purposes of localizing the compositions of the instant invention to target tissues.

The term “targeting moiety” (abbreviated “TM”), as used herein, is specifically intended to include antibodies, antibody fragments, the categories of binding molecules listed in Table 1, or peptides, hormones, or organic molecules that have specific binding affinity for a target ligand such as cell-surface receptors or antigens or glycoproteins, oligonucleotides, enzymatic substrates, antigenic determinants, or binding sites that may be present in the on the surface of a target tissue or cell. In some embodiments, a TM is non-proteinaceous. Non-limiting examples of non-proteinaceous TMs are provided herein, such as folate.

The terms “antigen”, “target antigen” and “immunogen” are used interchangeably herein to refer to the structure or binding determinant that an antibody, antibody fragment or an antibody fragment-based molecule binds to or has specificity against.

The term “antagonist”, as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

A “target” refers to the ligand of a targeting moiety, such as cell-surface receptors, antigens, glycoproteins, oligonucleotides, enzymatic substrates, antigenic determinants, or binding sites that may be present in the on the surface of a target tissue or cell.

A “target tissue” refers to a tissue that is the cause of or is part of a disease condition such as, but not limited to cancer or inflammatory conditions. Sources of diseased target tissue include a body organ, a tumor, a cancerous cell, bone, skin, cells that produce cytokines or factors contributing to a disease condition.

A “defined medium” refers to a medium comprising nutritional and hormonal requirements necessary for the survival and/or growth of the cells in culture such that the components of the medium are known. Traditionally, the defined medium has been formulated by the addition of nutritional and growth factors necessary for growth and/or survival. Typically, the defined medium provides at least one component from one or more of the following categories: a) all essential amino acids, and usually the basic set of twenty amino acids plus cysteine; b) an energy source, usually in the form of a carbohydrate such as glucose; c) vitamins and/or other organic compounds required at low concentrations; d) free fatty acids; and e) trace elements, where trace elements are defined as inorganic compounds or naturally occurring elements that are typically required at very low concentrations, usually in the micromolar range. The defined medium may also optionally be supplemented with one or more components from any of the following categories: a) one or more mitogenic agents; b) salts and buffers as, for example, calcium, magnesium, and phosphate; c) nucleosides and bases such as, for example, adenosine and thymidine, hypoxanthine; and d) protein and tissue hydrolysates.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Inhibition constant”, or “Ki”, are used interchangeably and mean the dissociation constant of the enzyme-inhibitor complex, or the reciprocal of the binding affinity of the inhibitor to the enzyme.

As used herein, “treat” or “treating,” or “palliating” or “ameliorating” are used interchangeably and mean administering a drug or a biologic to achieve a therapeutic benefit, to cure or reduce the severity of an existing condition, or to achieve a prophylactic benefit, prevent or reduce the likelihood of onset or severity the occurrence of a condition. By therapeutic benefit is meant eradication or amelioration of the underlying condition being treated or one or more of the physiological symptoms associated with the underlying condition such that an improvement is observed in the subject, notwithstanding that the subject may still be afflicted with the underlying condition.

A “therapeutic effect” or “therapeutic benefit,” as used herein, refers to a physiologic effect, including but not limited to the mitigation, amelioration, or prevention of disease in humans or other animals, or to otherwise enhance physical or mental wellbeing of humans or animals, resulting from administration of a polypeptide of the invention other than the ability to induce the production of an antibody against an antigenic epitope possessed by the biologically active protein. For prophylactic benefit, the compositions may be administered to a subject at risk of developing a particular disease, condition or symptom of the disease (e.g., a bleed in a diagnosed hemophilia A subject), or to a subject reporting one or more of the physiological symptoms of a disease, even though a diagnosis of this disease may not have been made.

The terms “therapeutically effective amount” and “therapeutically effective dose”, as used herein, refer to an amount of a drug or a biologically active protein, either alone or as a part of a polypeptide composition, that is capable of having any detectable, beneficial effect on any symptom, aspect, measured parameter or characteristics of a disease state or condition when administered in one or repeated doses to a subject. Such effect need not be absolute to be beneficial. Determination of a therapeutically effective amount is well within the capability of those skilled in the art, especially in light of the detailed disclosure provided herein.

The term “therapeutically effective dose regimen”, as used herein, refers to a schedule for consecutively administered multiple doses (i.e., at least two or more) of a biologically active protein, either alone or as a part of a polypeptide composition, wherein the doses are given in therapeutically effective amounts to result in sustained beneficial effect on any symptom, aspect, measured parameter or characteristics of a disease state or condition.

### I). General Techniques

The practice of the present invention employs, unless otherwise indicated, conventional techniques of immunology, biochemistry, chemistry, molecular biology, microbiology, cell biology, genomics and recombinant DNA, which are within the skill of the art. See Sambrook, J. et al., “Molecular Cloning: A Laboratory Manual,” 3^{rd} edition, Cold Spring Harbor Laboratory Press, 2001; “Current protocols in molecular biology”, F. M. Ausubel, et al. eds.,1987; the series “Methods in Enzymology,” Academic Press, San Diego, Calif.; “PCR 2: a practical approach”, M. J. MacPherson, B. D. Hames and G. R. Taylor eds., Oxford University Press, 1995; “Antibodies, a laboratory manual” Harlow, E. and Lane, D. eds., Cold Spring Harbor Laboratory,1988; “Goodman & Gilman's The Pharmacological Basis of Therapeutics,” 11^{th} Edition, McGraw-Hill, 2005; and Freshney, R. I., “Culture of Animal Cells: A Manual of Basic Technique,” 4^{th} edition, John Wiley & Sons, Somerset, N.J., 2000, the contents of which are incorporated in their entirety herein by reference.

Host cells can be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing eukaryotic cells. In addition, mammalian host cells can be grown in a defined medium that lacks serum but is supplemented with hormones, growth factors or any other factors necessary for the survival and/or growth of a particular cell type. Whereas a defined medium supporting cell survival maintains the viability, morphology, capacity to metabolize and potentially, capacity of the cell to differentiate, a defined medium promoting cell growth provides all chemicals necessary for cell proliferation or multiplication. The general parameters governing host cell survival and growth in vitro are well established in the art. Physicochemical parameters which may be controlled in different cell culture systems are, e.g., pH, pO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, most cells also require one or more hormones from at least one of the following groups: steroids, prostaglandins, growth factors, pituitary hormones, and peptide hormones to proliferate in serum-free media (Sato, G. H., et al. in “Growth of Cells in Hormonally Defined Media”, Cold Spring Harbor Press, N.Y., 1982). In addition to hormones, cells may require transport proteins such as transferrin (plasma iron transport protein), ceruloplasmin (a copper transport protein), and high-density lipoprotein (a lipid carrier) for survival and growth in vitro. The set of optimal hormones or transport proteins will vary for each cell type. Most of these hormones or transport proteins have been added exogenously or, in a rare case, a mutant cell line has been found which does not require a particular factor. Those skilled in the art will know of other factors required for maintaining a cell culture without undue experimentation.

Growth media for growth of prokaryotic host cells include nutrient broths (liquid nutrient medium) or LB medium (Luria Bertani). Suitable media include defined and undefined media. In general, media contains a carbon source such as glucose needed for bacterial growth, water, and salts. Media may also include a source of amino acids and nitrogen, for example beef or yeast extract (in an undefined medium) or known quantities of amino acids (in a defined medium). In some embodiments, the growth medium is LB broth, for example LB Miller broth or LB Lennox broth. LB broth comprises peptone (enzymatic digestion product of casein), yeast extract and sodium chloride. In some embodiments, a selective medium is used which comprises an antibiotic. In this medium, only the desired cells possessing resistance to the antibiotic will grow.

### II). XTEN and Targeted Cytotoxic Conjugate Compositions

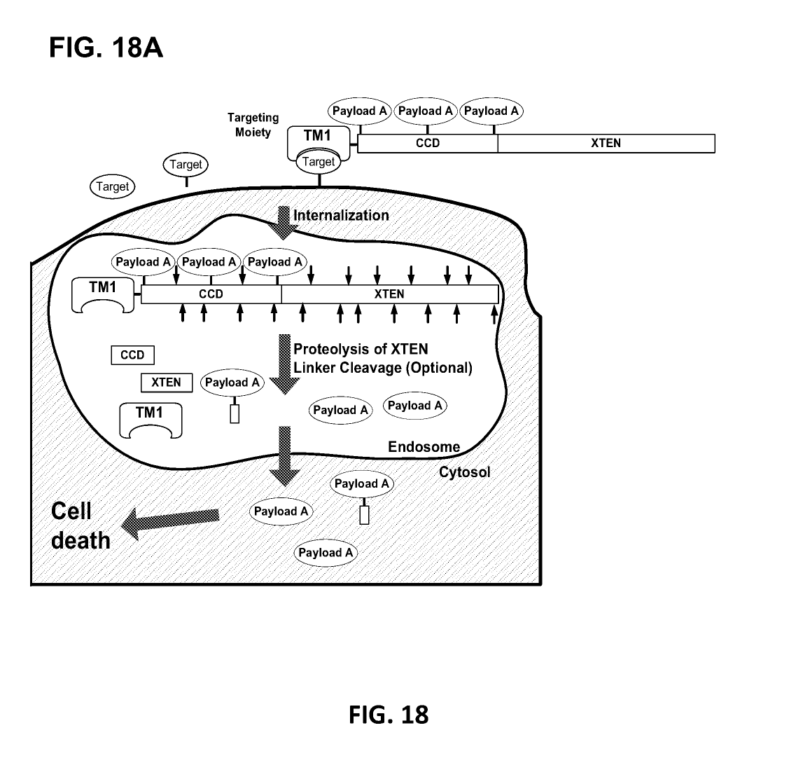

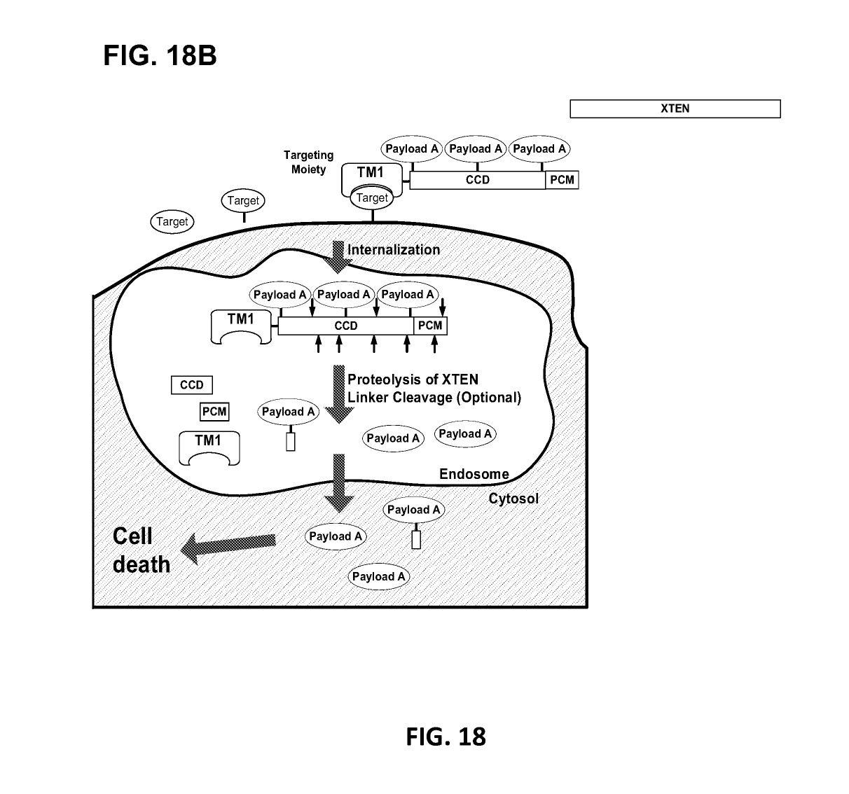

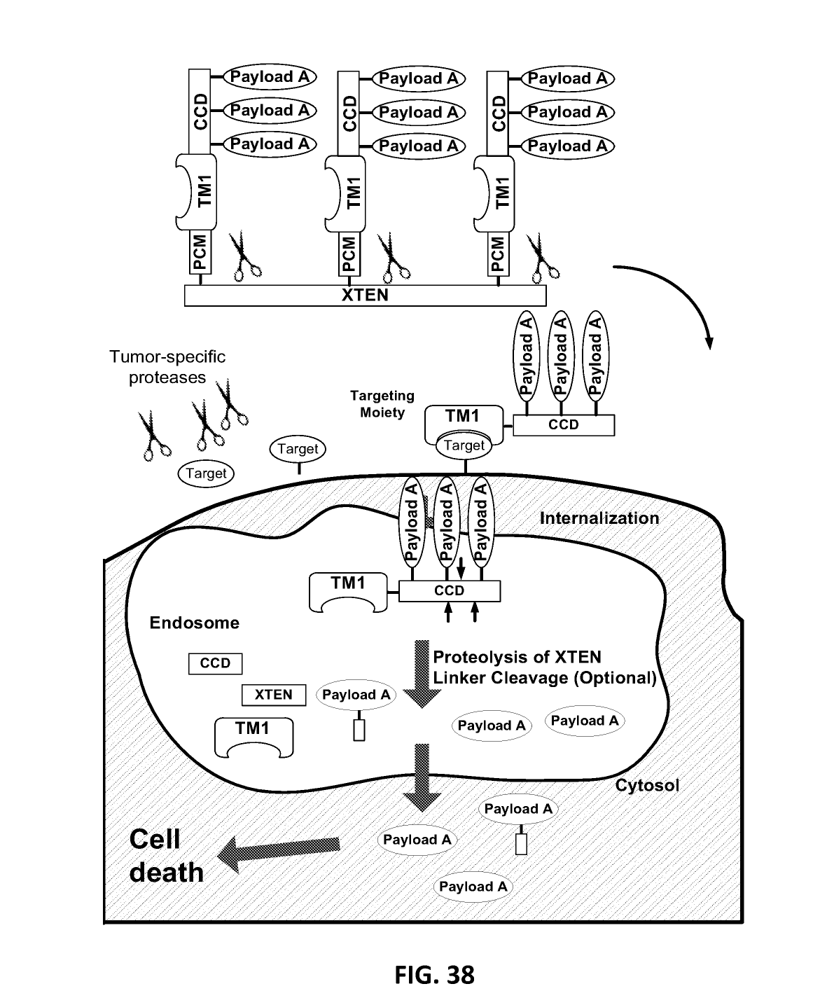

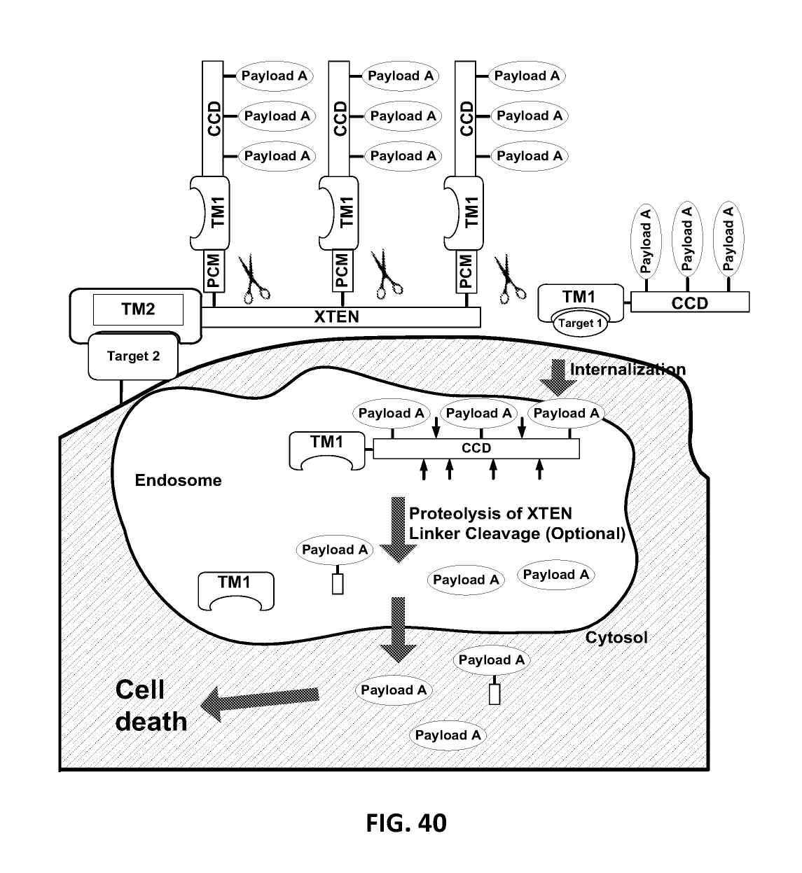

The present invention relates, in part, to targeted conjugate compositions comprising drug payloads capable of selectively binding a target tissue such as a tumor or cancer cell or inflammatory tissue, such that the drug component is taken up by the targeted cell, thereby effecting the pharmacologic effect, wherein the composition comprises one or more XTEN, which confers shielding and enhanced pharmacokinetic and pharmaceutical properties. The invention contemplates several different configurations of the compositions in order to confer certain properties on the subject compositions. In a first type of configuration, the conjugate compositions comprise a fusion protein of a first short polypeptide portion comprising hydrophilic amino acids interspersed with cysteine residues (referred to hereafter as a cysteine containing domain, or CCD) fused to a second portion longer than said first portion that comprises an XTEN polypeptide, and a third portion comprises a targeting moiety (TM) that is capable of specifically binding a ligand associated with the target tissue, and pharmacologically active drugs or biologics (including cytotoxic drugs capable of killing the cells bearing the target cell ligand or anti-inflammatory drugs) conjugated to the cysteine residues of the CCD wherein the targeting moiety binds to the targeted cell and is internalized and degraded, releasing the drug or biologic to exert its pharmacologic effect. In a second type of configuration, the targeted conjugate composition has, in addition to the foregoing components, a protease cleavage moiety (PCM) inserted recombinantly between the CCD and the XTEN, wherein the PCM is capable of being cleaved by a mammalian protease associated with or in proximity to the target tissue. In such case, when the composition is in proximity to, or is bound to the target tissue or cell, the XTEN is released from the composition by the action of the protease, greatly reducing the size of the remaining portion of the construct (the remaining portion hereinafter refered to as a “released targeted composition”, which comprises the one or more targeting moieties fused or linked to the CCD and the drug or biologic linked to the CCD) and shielding effect imparted by the XTEN such that the released targeted composition having the TM and CCD with the attached drugs is better able to extravasate and penetrate the target tissue and be taken up by the cell bearing the ligand of the targeting moiety, whereupon by the internal processing of the molecule, the released drugs exert their pharmacologic effect (see e.g. FIGS. 18, 38 and 40 for a schematic representation of the foregoing process). Thus, the second type is designed to be utilized as a form of prodrug in that the compositions with the release of the shielding XTEN, and the released targeted composition becomes more active than the intact composition, more selective, are better able to extravasate, are better able to penetrate the target tissue, and has higher binding affinity due to the loss of the shielding effect and/or steric hinderance. In an advantage of these compositions, because of the shielding and bulking effects of the XTEN, the intact composition is less likely to interact or bind to normal tissues (that may have a low frequency of receptors that are ligands for the TM, compared to the target tissue) and is less likely to extravasate from normal vasculature in healthy organs and tissues, resulting in less toxicity and fewer side effects compared to conventional chemotherapeutic or biologics. Once the intact composition is are cleaved by the proteases found colocalized in association with the target tissues, the released targeted composition is no longer shielded and regains its full binding affinity potential and because of its much smaller size, can more easily extravasate and penetrate the target tissue. Such compositions are useful in the treatment of certain diseases, including, but not limited to cancer and certain inflammatory diseases. The invention contemplates additional configurations, include variations of the foregoing, including constructs with two or more targeting moieties (which may be identical or may target different ligands), two or more XTEN to further increase the shielding effect and/or increase the molecular mass of the composition, two or more types of drug molecules linked to different CCD, or two or more PCM. In the some embodiments, the CCD, the XTEN, and the PCM (if present) are produced as a fusion protein, while the TM may be joined to the construct either recombinantly or by chemical conjugation. In other embodiments, the TM, the CCD, and the PCM (if present) are produced as a fusion protein, while the one or more XTEN may be joined to the construct either recombinantly or by chemical conjugation. In all cases, the drug or biologic payload is chemically conjugated to the CCD as described more fully, below.

1. Conjugates Linked to Cytotoxic Payloads, Targeting Moieties and Peptidic Cleavage Moieties

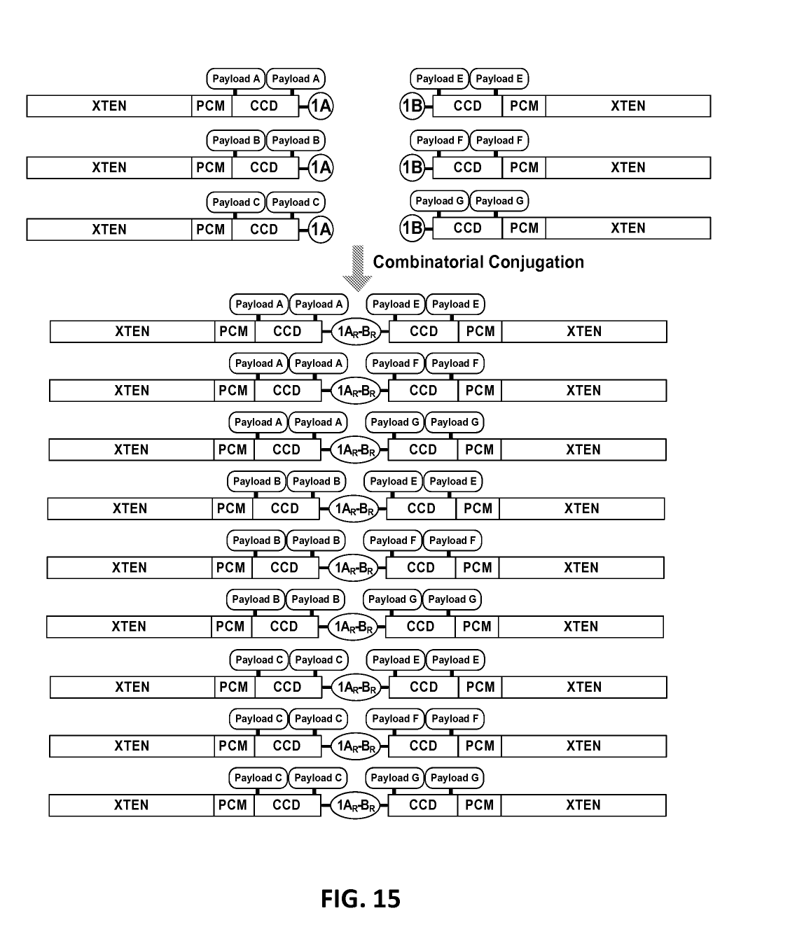

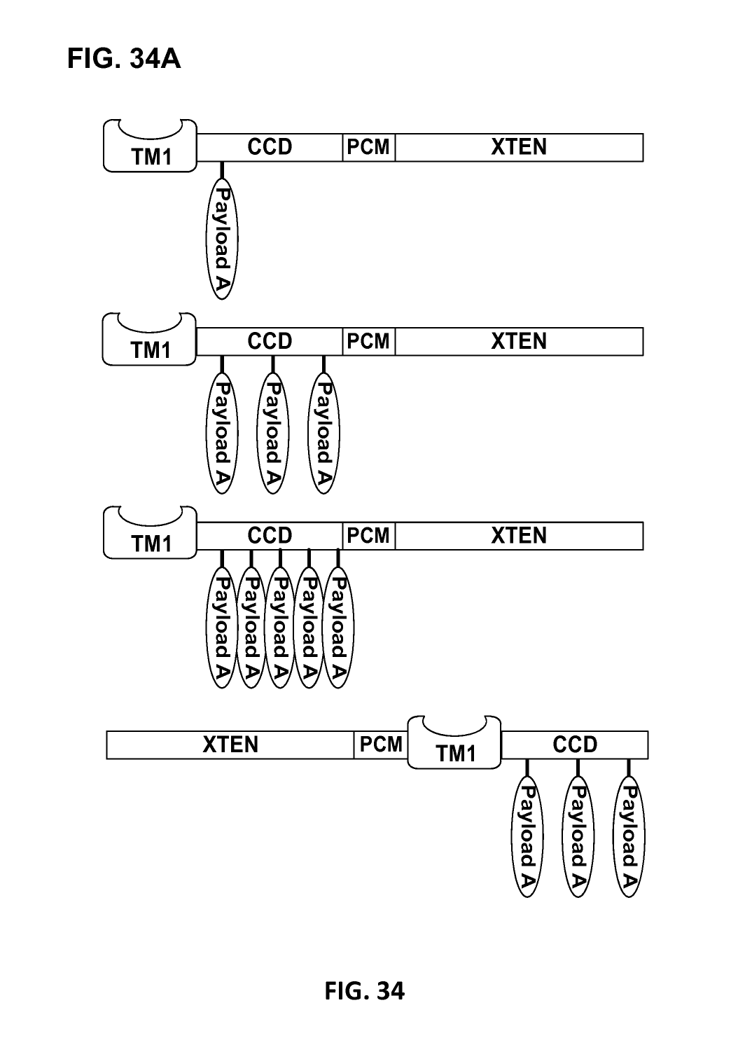

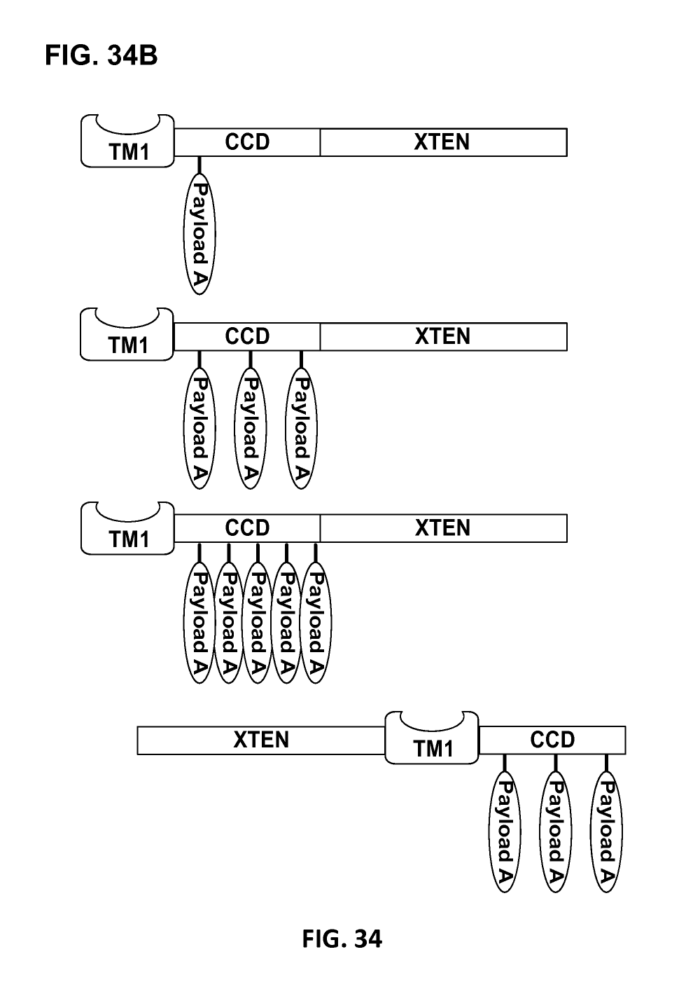

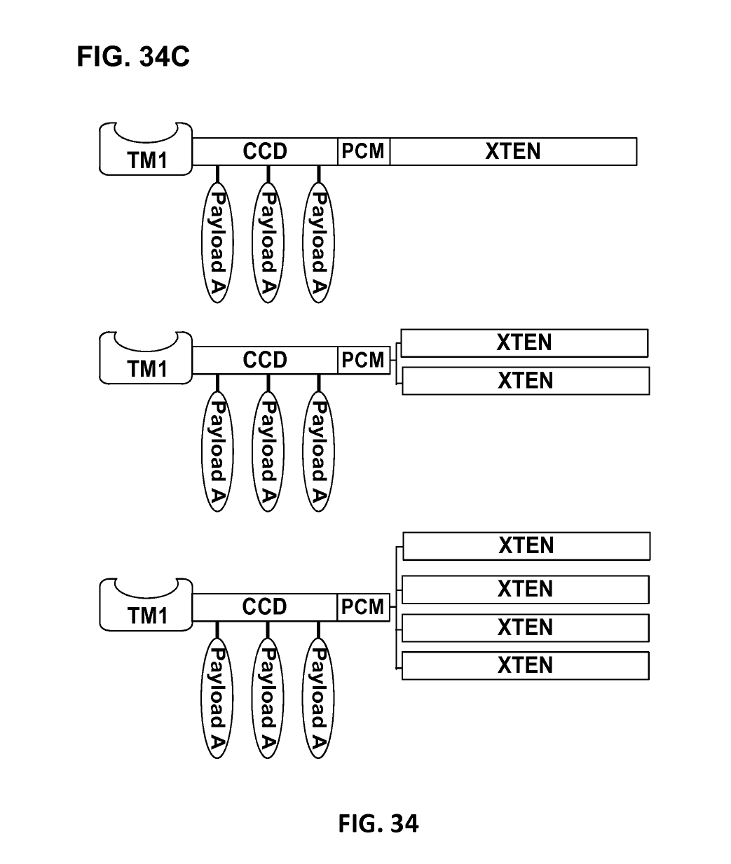

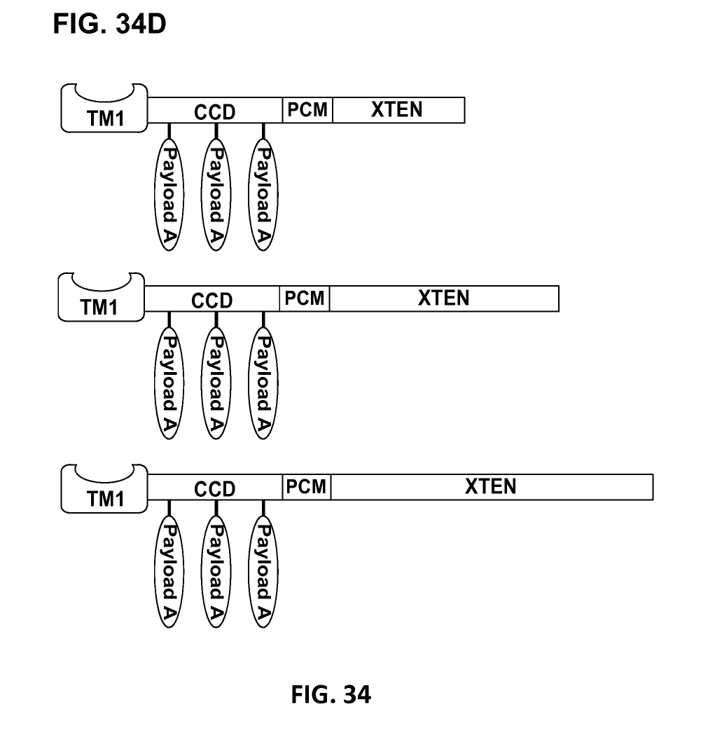

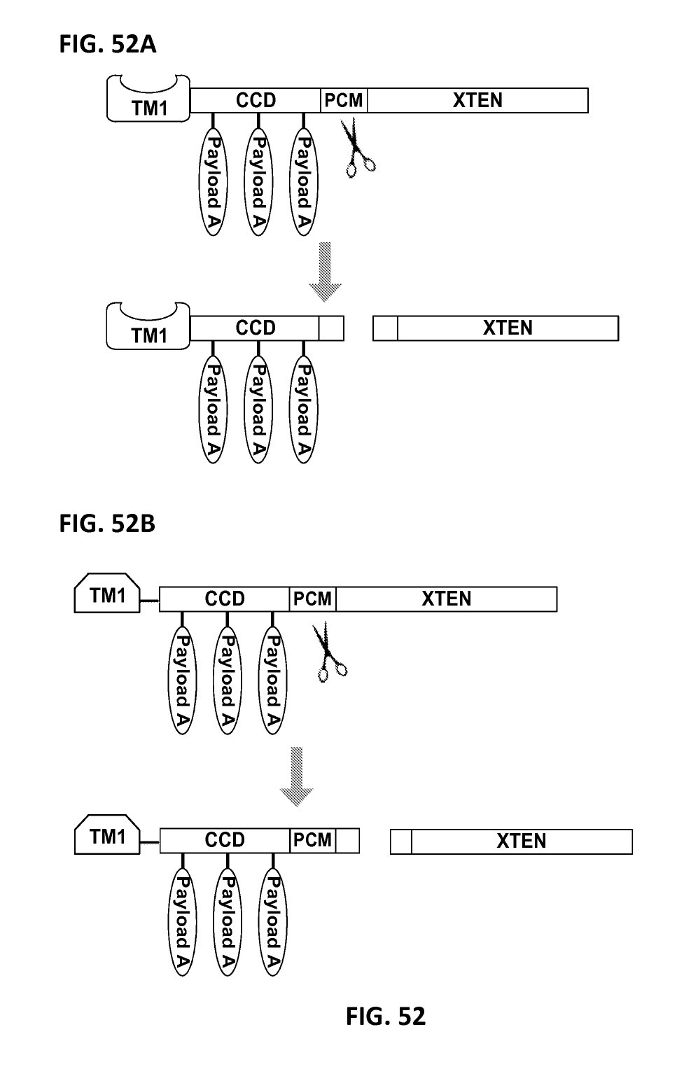

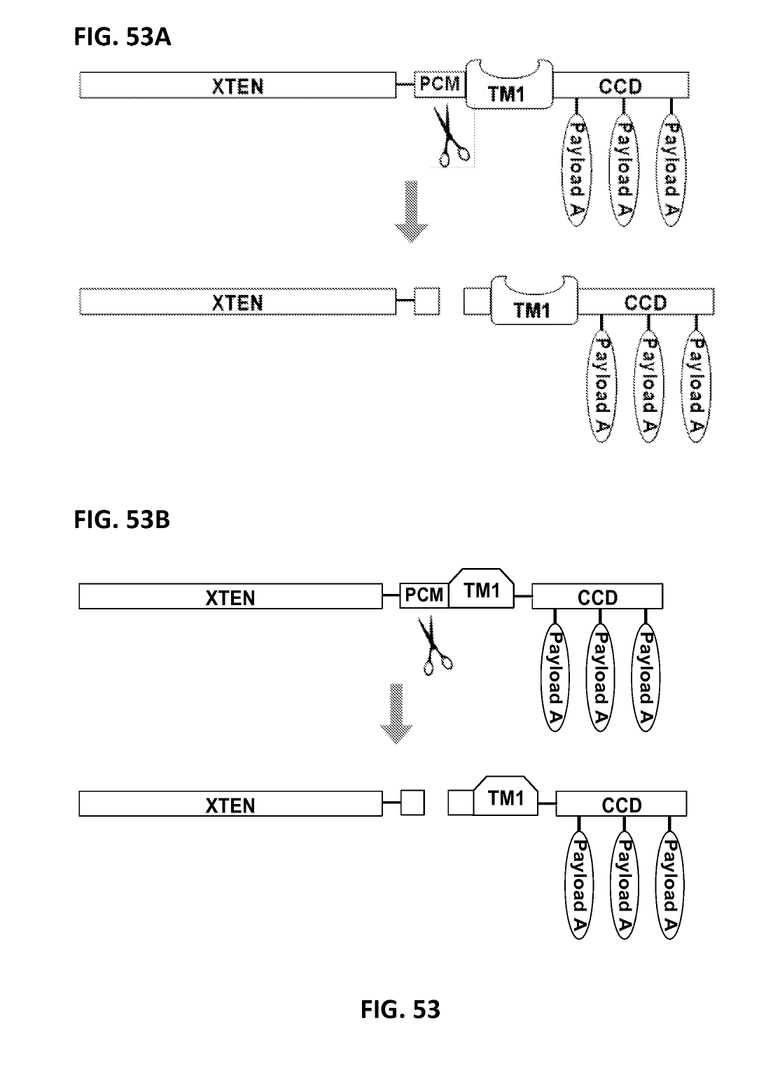

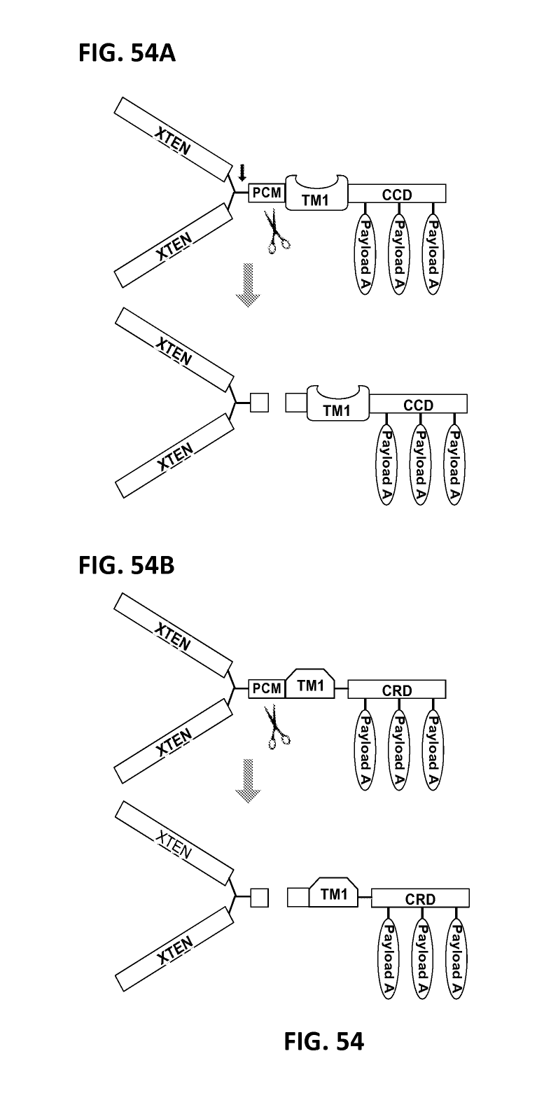

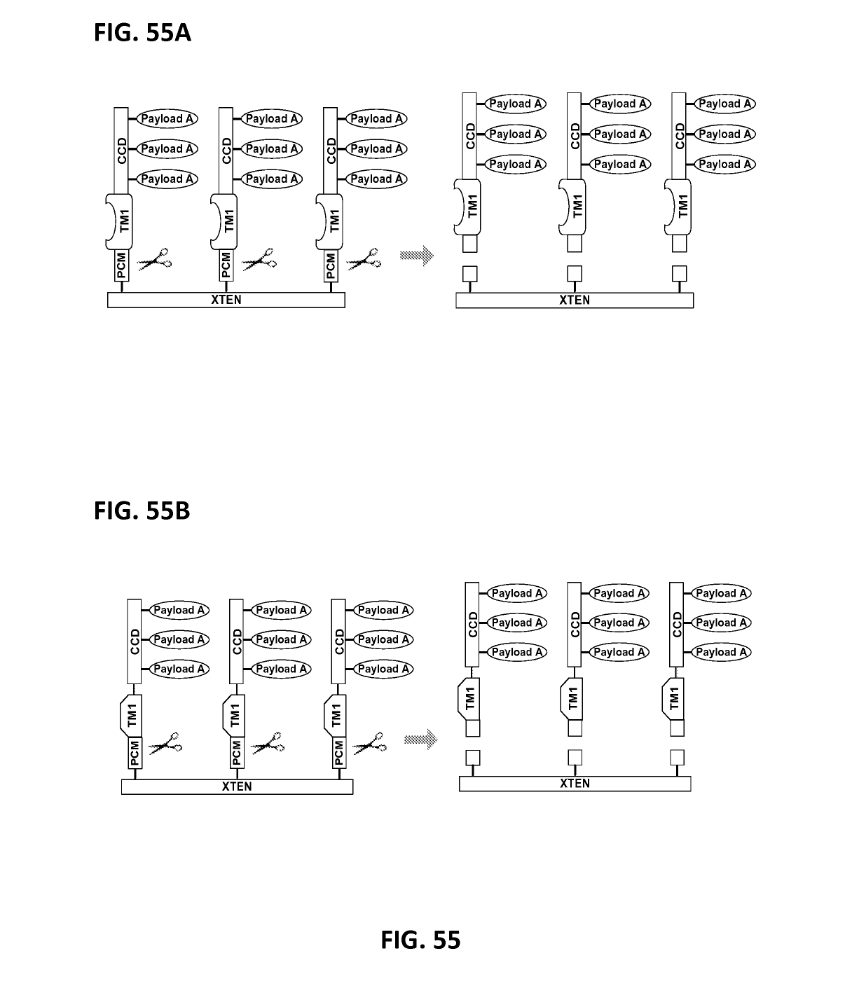

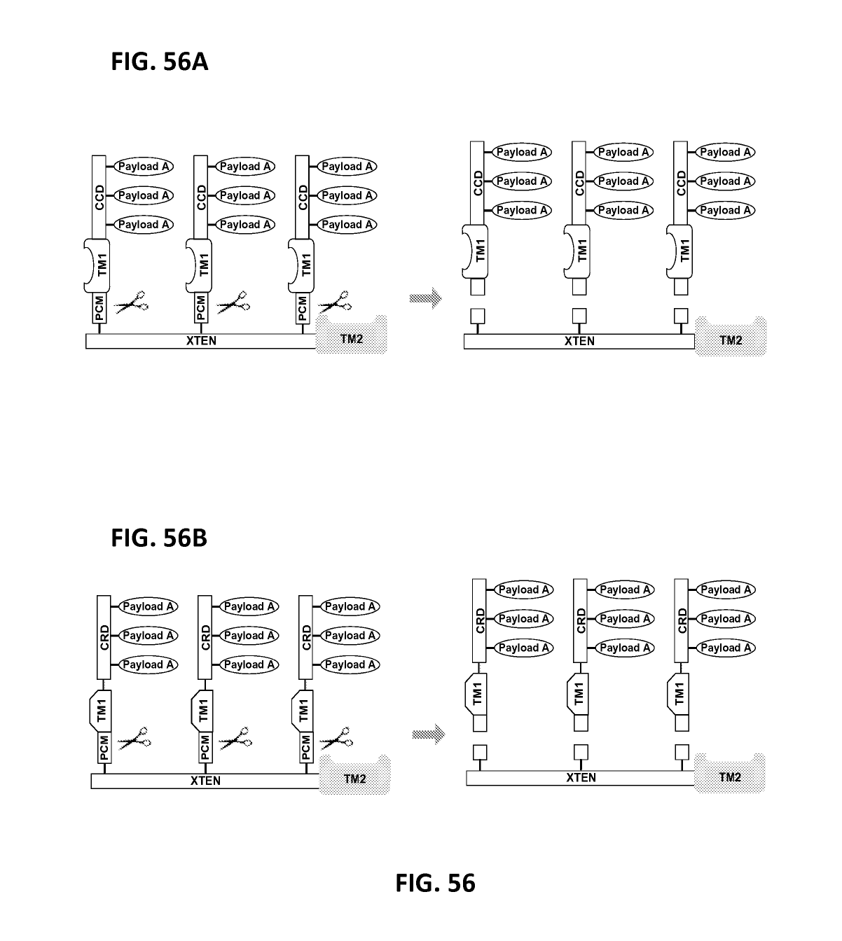

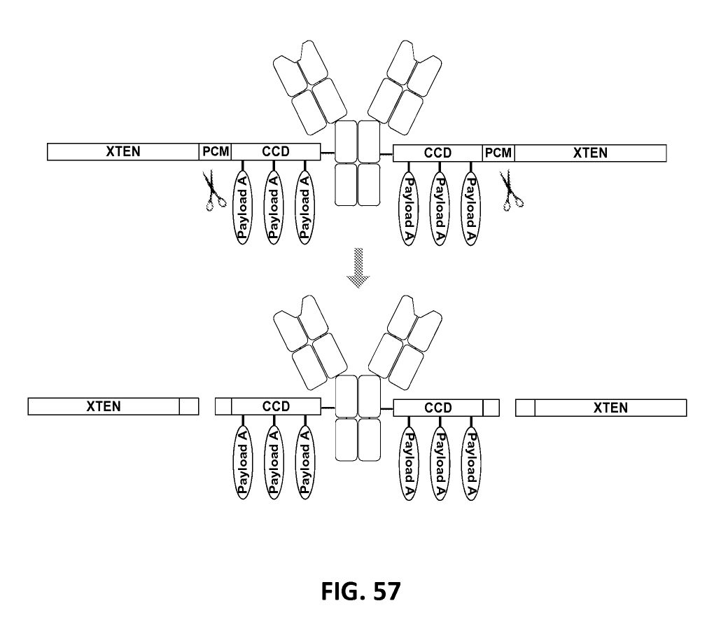

In one aspect, the instant invention provides targeted conjugate compositions comprising a cysteine containing domain (CCD) conjugated to pharmacologically active small molecules or biologics (e.g. biologically active proteins), one or more XTEN, one or more targeting moieties (TM), and one or more peptidic cleavage sequences (PCM), either linked together recombinantly or wherein some components are conjugated to the composition. The invention contemplates a diversity of configurations for use in the subject compositions, including, but not limited to the configurations illustrated in the various schematic drawings of the disclosure. The configurations are designed to confer certain properties to the resulting compositions, including the shielding of the TM and/or the cytotoxic payload drug (non-limiting examples of which are shown in FIGS. 34, 35, 37 and 39) by the attached large XTEN component, an increased molecular weight and hydrodynamic radius that confers enhanced pharmacokinetics and reduces extravasation into normal tissues, and the subsequent reduction of molecular size and hydrodynamic radius after cleavage of the PCM, releasing the large XTEN, such that there is an enhanced ability of the released components comprising the joined TM and CCD-payload conjugates (the “released targeted composition”) to extrasavate and penetrate into the target tissue (non-limiting examples of which are shown in FIGS. 52-57), and the targeting moiety regains its full binding affinity potential (after the shielding effect of the released XTEN), thereby selectively delivering the attached payload drugs to the targeted cell to exert its pharmacologic effect. In another aspect, the design of the configurations also provides the ability to provide cost-effective methods of making combinatorial compositions of various permutations of TM and payload drugs, non-limiting examples of which are shown in FIGS. 15-17, in order to increase potency, safety, and efficacy.

In another aspect, it is an object of the invention to provide targeted conjugate compositions that have the CCD and linked drug payloads, the XTEN, and the TM with binding affinity to the target tissue, but that are lacking the PCM. It is contemplated that in applications where either penetration into the tissue is not a limiting factor (e.g., blood cancers or in diseased tissues with leaky vasculature) or in those disorders where a suitable protease is not produced, the targeted conjugate compositions without the PCM nevertheless have the ability to bind to the target tissue ligand thereby delivering the drug payload, resulting in the desired pharmacologic effect, yet still have the benefit of the enhanced pharmacokinetic properties conferred by the attached XTEN.

In yet another aspect, it is an object of the invention to provide targeted conjugate compositions that have all of the above described components but are configured to include a second, different drug payload, resulting in a bifunctional composition that can provide multiple pharmacologic effects, thereby increasing the overall therapeutic effect. Generally, such compositions will comprise two or three CCD and fused PCM and XTEN arranged in a branched or multimeric configuration, as described more fully, below.

In another aspect, it is an object of the invention to provide targeted conjugate compositions designed with configurations of multiple copies of the TM, CCD and linked payloads and XTEN such that the payloads and/or the TM are shielded by the multiple XTEN components in order to reduce or eliminate non-specific interactions with tissues or cells that are not the intended targets of the compositions, thereby reducing undesireable toxicity or side effects. It will be appreciated by those of skill in the art that the some compositions of the instant invention achieve this reduction in non-specific interactions by a combination of mechanims, which include steric hinderance by locating the TM and/or payloads proximal to the points of attachment between the bulky XTEN molecules, in that the flexible, unstructured characteristic of the long flexible XTEN polypeptides, by being tethered to the composition, are able to oscillate and move around the TM and payload components, providing a degree of blocking between the composition and tissues or cells, as well as a reduction in the ability of the intact composition to penetrate a cell or tissue due to the large molecular mass (contributed to by both the actual molecular weight of the XTEN and due to the known property of the large hydrodynamic radius of the unstructured XTEN) compared to the size of the individual TM and payload components. However, these compositions are designed such that when in proximity to a target tissue or cell bearing or secreting a protease capable of cleaving the PCM, the TM and linked payload is liberated from the bulk of the XTEN by the action of the protease(s), removing the steric hindrence barrier, and is freer to bind to and be internallized by the targeted cell and exert the pharmacologic effect of the attached payload drugs or biologics. The subject compositions fmd use in the treatment of a variety of conditions where selective delivery of a therapeutic or toxic payload to a cell, tissue or organ is desired. In one embodiment, the target tissue is a cancer, which may be a leukemia, a lymphoma, or a tumor. In another embodiment, the target tissue is an area of inflammation, which may be localized in an organ or is generalized in the subject. It is contemplated that the compositions comprising anti-inflammatory drugs or biologics can be used in treatment of diseases selected from the group consisting of acne vulgaris, asthma, autoimmune diseases, autoinflammatory diseases, celiac disease, chronic prostatitis, glomerulonephritis, hypersensitivity reaction, inflammatory bowel disease, Crohn's disease, pelvic inflammatory disease, reperfusion injury, rheumatoid arthritis, sarcoidosis, transplant rejection, vasculitis, psoriasis, fibromyalgia, irritable bowel syndrome, lupus erythematosis, osteoarthritis, scleroderma, and ulcerative colitis.

The invention contemplates a diversity of targeting moieties for use in the subject compositions, including antibodies, antibody fragments such as but not limited to scFV, and antibody mimetics including, but not limited to those set forth in Table 1, as well as peptides and synthetic molecules capable of binding ligands or receptors associated with disease or metabolic or physiologic abnormalities such as, but not limited, to folate, asparaginylglycylarginine analogs (NGR), arginylglycylaspartic acid analogs (RGD) and LHRH analogs described herein. Some of the compositions of the instant invention comprising PCM are designed with consideration of the location of the target tissue protease as well as the presence of the same protease in healthy tissues not intended to be targeted, as well as the presence of the target ligand in healthy tissue but a higher degree of presence of the ligand in unhealthy target tissue, in order to provide the widest therapeutic window (as defined by the largest difference between the minimal effective dose and the maximal tolerated dose) for the composition. To achieve the widest therapeutic window, it is specifically contemplated that some embodiments of the invention provide compositions wherein the TM of the compositions will be placed at an internal location within the composition (rather than at a terminal location) where it can be partially shielded by the XTEN that surrounds it (e.g., where the ligand is found in both healthy tissues and unhealthy target tissues but is in higher concentrations in the latter). Similarly, in order to achieve the widest therapeutic window, it is specifically contemplated that some embodiments of the invention provide compositions wherein the cytotoxic payload is either shielded by the XTEN or linked by PCM to the CCD such that the payload drugs are not released from the composition until the composition is in contact with the target tissue protease or is internalized by the target cell in order to reduce the effects of the payload on healthy tissue.

Conversely, where there is a lower degree of presence of the target ligand in healthy tissue, the invention provides configuration embodiments in which the TM will be incorporated in higher numbers in the composition or in a location less likely to be shielded by the XTEN (such as on the N- or C-terminus of the composition) such that the targeted conjugate composition can efficiently reach and be specifically accumulated in the unhealthy target tissue.

In preferred embodiments, the targeted conjugate compositions are designed such that the TM and the payload remain connected to each other after the PCM is cleaved by one or more tissue-associated proteases and is cleaved away from the XTEN of the composition, with the resulting effect that the smaller mass of the TM and the joined CCD-payload fragment (a “released targeted comosition”) is more effectively able to penetrate into the target tissue and bind to the cell ligand of the TM and then be internalized in the diseased cell in order to exert the pharmacologic effect of the payload (see FIGS. 18B, 38 and 40). In some embodiments, the targeted conjugate compositions are designed with a PCM wherein the PCM is a substrate for two or more different extracellular proteases, each capable of cleaving the composition into a fragment that comprises the TM linked to the joined CCD-payload portion that will bind to the ligand and be taken internalized in the target tissue, whereupon the payload exerts its pharmacologic effect. In some other embodiments, the targeted conjugate compositions are designed with a first and a second PCM wherein the each PCM is a substrate for a different extracellular protease that is capable of cleaving the composition into a fragment that contains the TM, or a fragment that comprises the payload, or a fragment that comprises the TM linked to a payload that will bind to the ligand and be internalized in the target tissue, whereupon the payload exerts its pharmacologic effect. The foregoing embodiments take advantage of the fact that certain diseased target tissues are capable of expressing more than one protease, and that by the selective introduction of different PCM susceptible to different proteases into the compositions, the resulting composition is more likely to be cleaved in proximity to the diseased tissue relative to healthy tissue. In such embodiments, it will be appreciated that the effects on healthy tissues would be lessened by the design of the composition wherein the TM and/or the payload is shielded by flanking XTEN or by designing the TM and/or payload components to be sterically hindered by their location within the construct until reaching the targeted tissue and the associated protease of the target tissue.

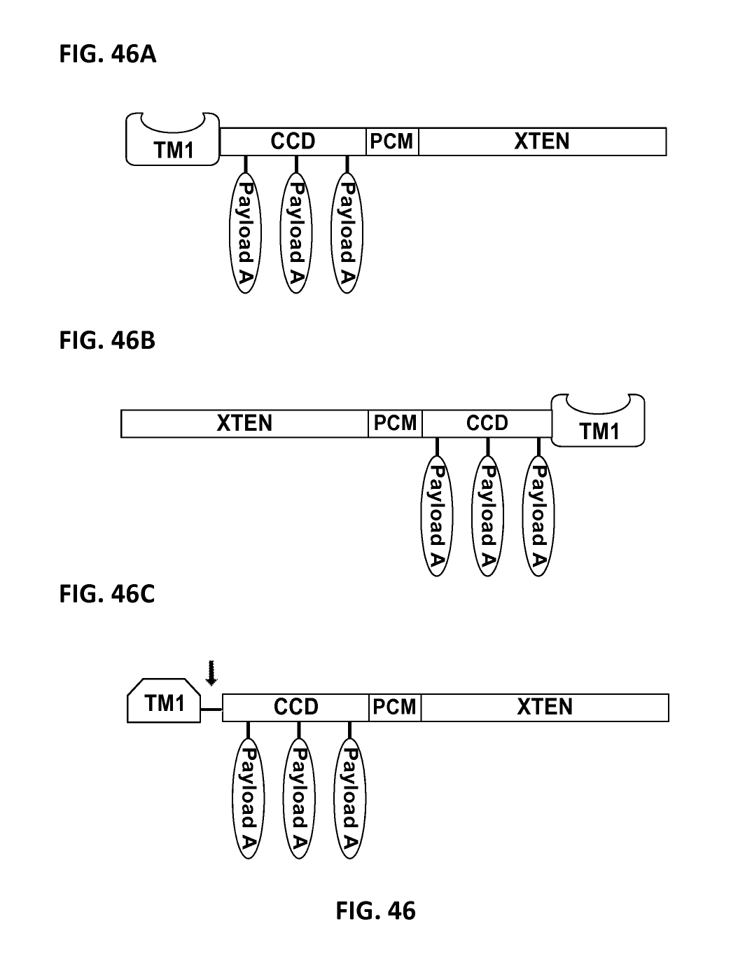

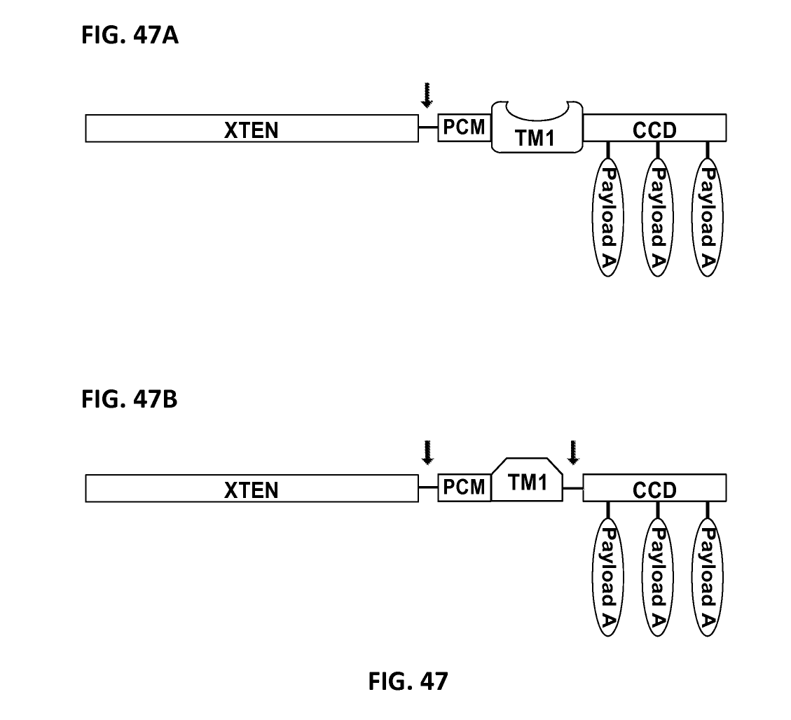

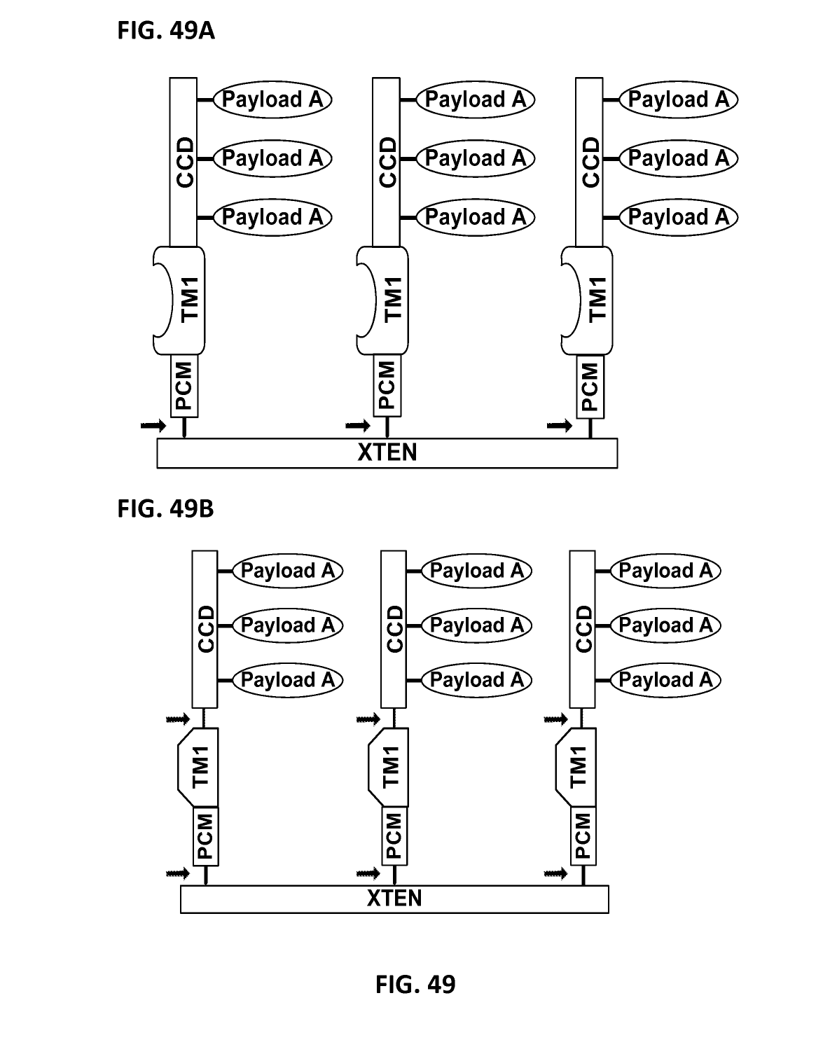

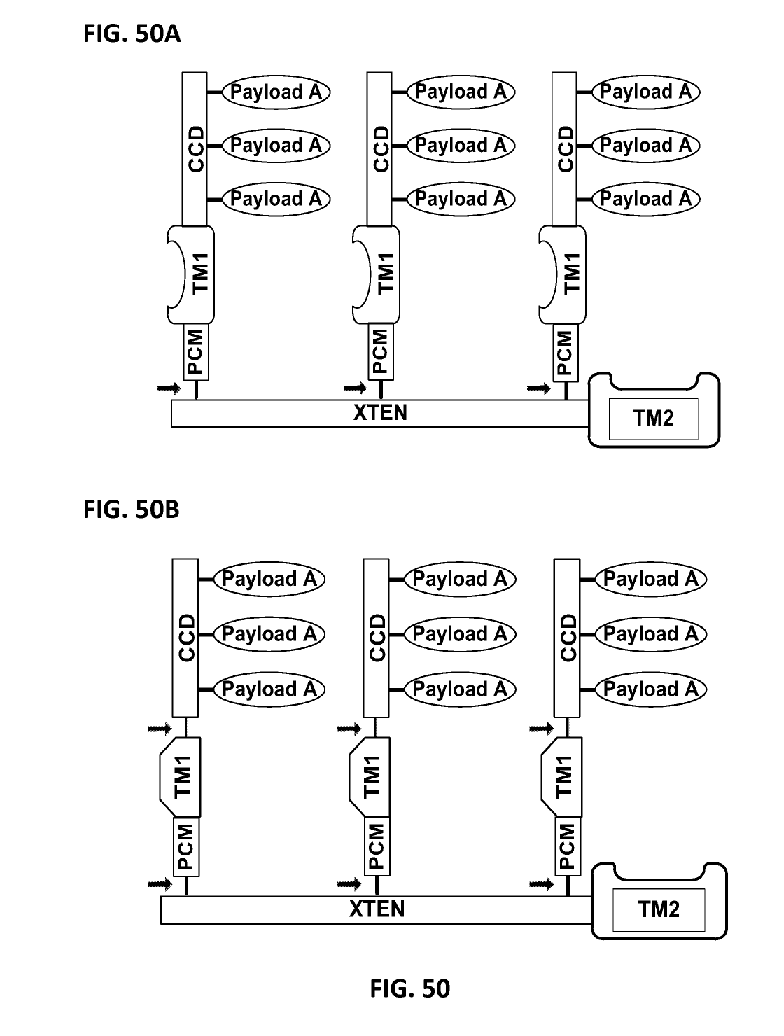

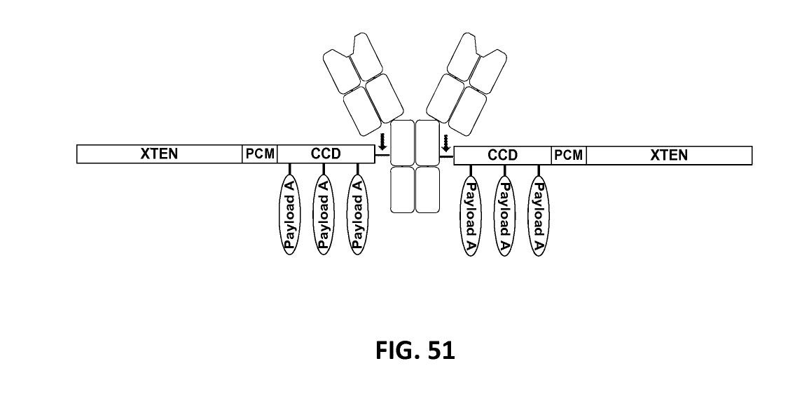

In certain embodiments, the disclosure provides targeted cleavable conjugate compositions comprising a single fusion protein having a short first portion comprising a TM, a cysteine containing domain (CCD) and a peptidic cleavage moiety (PCM) that is a substrate for one or more proteases associated with a target tissue, wherein the PCM is recombinantly linked to a longer second portion comprising an XTEN sequence, separating the construct into two regions; a first region in which the CCD and the linked drug payloads is joined to one or more molecules of a targeting moiety (e.g., either recombinantly or by conjugation) and a second region comprising the XTEN. Non-limiting examples of the resulting compositions are portrayed schematically in FIGS. 46-51. The construct can be designed to be in various configurations from the N-terminus to the C-terminus, including (TM)-(CCD)-(PCM)-(XTEN); (XTEN)-(PCM)-(CCD)-(TM); (XTEN)-(PCM)-(TM)-(CCD); and (CCD)-(TM)-(PCM)-(XTEN). In one embodiment of the foregoing, the CCD sequence exhibits at least 90%, or 91%, or 92%, or 93%, or 94%, or 95%, or 97%, or 99% identity or is identical to a sequence set forth in Table 6, the PCM is a sequence selected from the sequences set forth in Table 8, and the XTEN exhibits at least 90%, or 91%, or 92%, or 93%, or 94%, or 95%, or 96%, or 97%, or 98%, or 99% identity or is identical to a sequence set forth in Table 10. In some embodiments of the subject compositions, the one or more molecules of the TM are antibody fragments. In one embodiment, the one or more molecules of the TM are scFV derived from the antibodies set forth in Table 19 or derived from the VL and VH sequences of Table 19. In another embodiment of the targeted conjugate compositions, the one or more molecules of the TM are non-proteinaceous or are small molecule receptor ligands. In some embodiments, the one or more non-proteinaceous TM are folate. In another embodiment of the targeted conjugate compositions, the one or more molecules of the TM are LHRH (including the analogs of Table 22). In another embodiment of the targeted conjugate compositions, the one or more molecules of the TM are RGD or RGD analogs or NGD or NGD analogs. In another embodiment of the targeted conjugate composition, the drug payloads are selected from the group of payloads of Tables 14-17. In another embodiment of the of the targeted conjugate compositions, the compositions comprise two different payloads wherein each is selected from the group of payloads of Tables 14-17. In another embodiment, the payloads are biologically active proteins, such as proteins selected from the group of payload of Table 16. In other embodiments, the payloads of the targeted compositions are cytotoxic drugs and are selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A. In one embodiment of the foregoing, the cytotoxic payload is MMAF. In another embodiment of the foregoing, the cytotoxic payload is maytansine. In another embodiment of the foregoing, the cytotoxic payload is paclitaxel. In another embodiment of the foregoing, the cytotoxic payload is Pseudomonas exotoxin. In another embodiment of the foregoing, the cytotoxic payload is MMAE. In another embodiment of the foregoing, the cytotoxic payload is mertansine (DM1). In other embodiments, the targeted conjugate composition comprise two different cytotoxic drugs selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A.

In one embodiment of the targeted conjugate composition, the peptidic cleavage moiety (PCM) of the composition is selected from the group of sequences set forth in Table 8. It is specifically contemplated that the PCM of a given compositions have a sequence that is a substrate for one or more proteases associated with a tissue wherein an antigen, marker or receptor on said tissue is also a ligand for the TM of that composition. In such embodiments, the binding of the TM to the ligand brings the targeted conjugate composition into proximity with the tissue-associated protease whereupon the composition is cleaved, thereby releasing the cytotoxic payloads proximal to or within said tissue, resulting in a pharmacologic effect of the drug component. In one embodiment, wherein the drug is a cytotoxic drug, the targeted conjugate composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a cell line comprising said tissue ligand compared to the toxicity of the composition when the cell line does not comprise said tissue ligand. In another embodiment, the composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a cell line comprising the tissue ligand and in which the target tissue-associated protease is present, compared to the toxicity of the composition when the assay does not have the protease. In another embodiment, the targeted conjugate composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay wherein the PCM is cleaved compared to the toxicity of the composition when PCM is not cleaved. In another embodiment, the released targeted composition comprising the TM and theCCD comprising the cytotoxic compound(s) that is cleaved and released from the composition is internalized into a target cell in an in vitro mammalian cell cytotoxicity assay at a concentration that is least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater compared the intact composition that is not cleaved. In another embodiment, the intact targeted conjugate composition exhibits a terminal half-life when administered to a subject that is 10-fold, or 20-fold, or 30-fold, or 40-fold, or 50-fold, or 100-fold longer compared to the cytotoxic drug not linked to the composition and administered in a comparable fashion to a subject. In another embodiment, the targeted conjugate composition exhibits a terminal half-life of at least about 3 days, or at least about 7 days, or at least about 10 days, or at least about 14 days, or at least about 21 days, or at least about 30 days when administered to a subject.

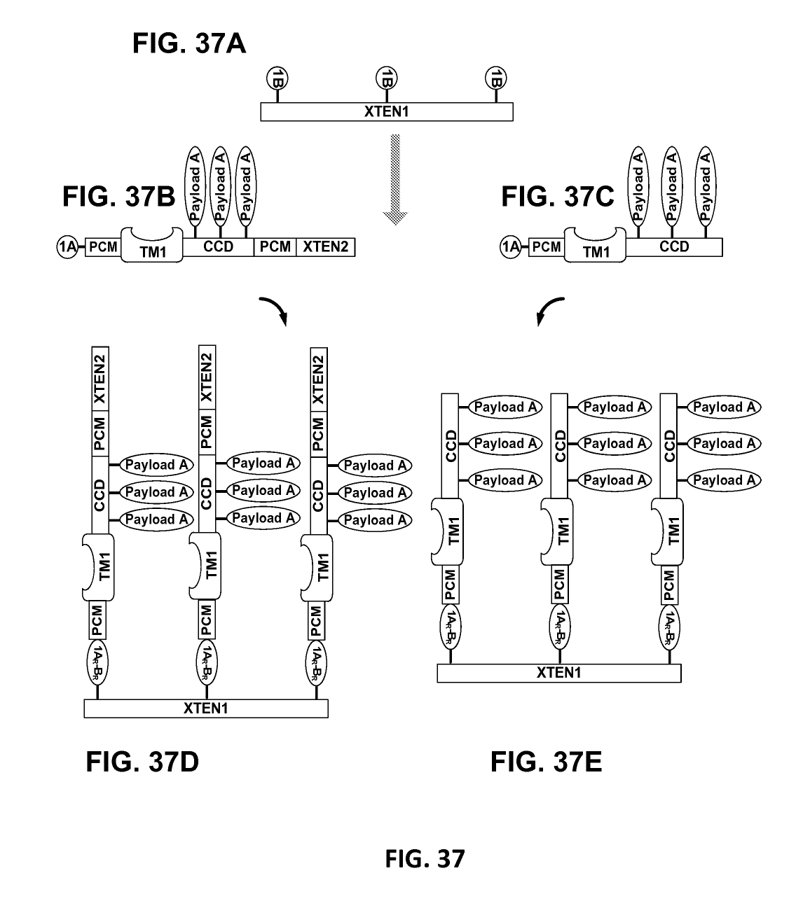

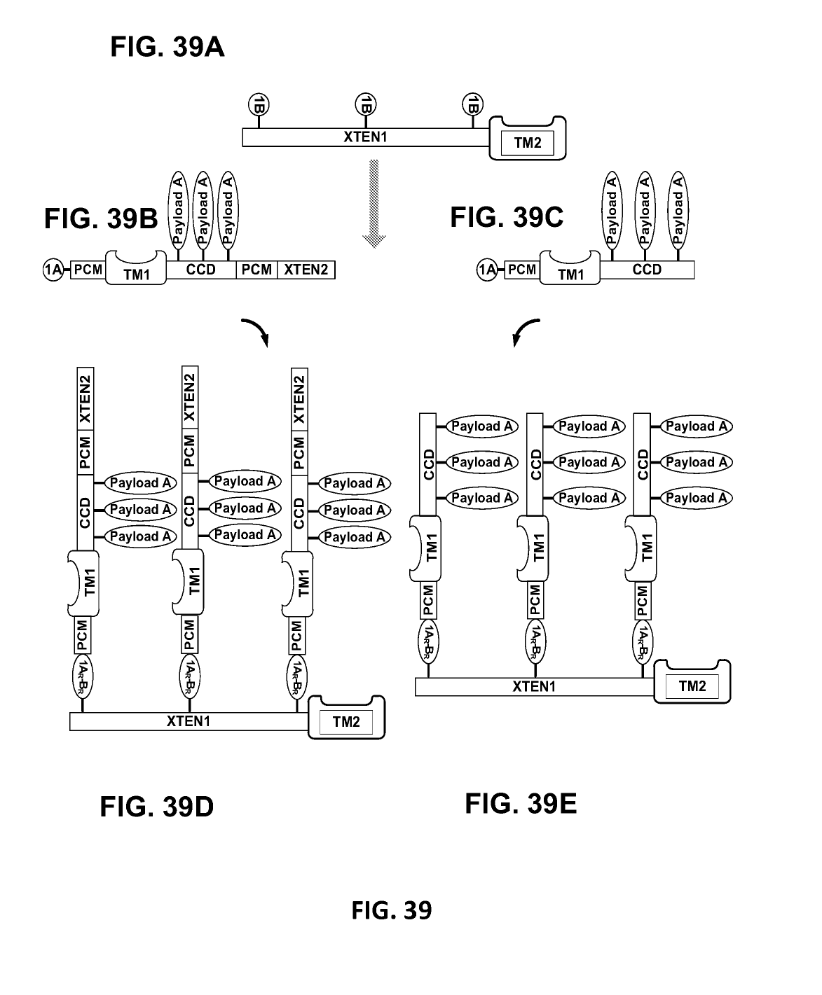

In another aspect, the invention provides multiple targeted conjugate compositions that are conjugated to an XTEN backbone having cysteine residues (e.g., a sequence of Table 11). Non-limiting examples of the various configurations of the resulting compositions are portrayed schematically in FIGS. 37-40, 49 and 50. In one embodiment, the composition comprises a first XTEN comprising cysteine residues, serving as a “backbone” wherein one or more fusion proteins are linked to the cysteines of the backbone XTEN that comprise, in order, a PCM fused or conjugated to a targeting moiety and a CCD bearing drug payloads (see FIG. 37). In another configuration of the foregoing embodiment, the fusion protein further comprises another PCM and an XTEN attached to the C-terminus of the CCD of each of the fusion proteins attached to the backbone XTEN. In another embodiment, the composition comprises a first XTEN comprising cysteine residues wherein a targeting moiety is recombinantly fused or linked by conjugation to the N-or C-terminus of the XTEN, serving as a “backbone” wherein one or more fusion proteins are linked to the cysteines of the backbone XTEN that comprise, in order, a PCM fused or conjugated to a targeting moiety and a CCD bearing drug payloads (see FIG. 39). In another configuration of the foregoing embodiment, the fusion protein further comprises another PCM and an XTEN attached to the C-terminus of the CCD of each of the fusion proteins attached to the backbone XTEN. In the foregoing compositions, the first XTEN exhibits at least 90%, or 91%, or 92%, or 93%, or 94%, or 95%, or 96%, or 97%, or 98%, or 99% or is identical to a sequence selected from the XTEN sequences of Table 11. In another embodiment of the foregoing targeted conjugate composition, the XTEN of the one or more side fusion proteins exhibits at least 90%, or 91%, or 92%, or 93%, or 94%, or 95%, or 96%, or 97%, or 98%, or 99% identity or is identical to a sequence selected from the XTEN sequences of Table 10. In the foregoing embodiment, the composition comprises at least two, or at least three, or at least four, or at least five, or at least six, or at least seven, or at least eight, or at least nine of the fusion proteins comprising the TM, PCM and CCD and conjugated drug molecules, wherein the side fusion proteins are linked to the thiol of the XTEN cysteine residues using cross-linkers described hererin, below. In one embodiment of the composition, the one or more molecules of the TM are antibody fragments, such as an scFv derived from the antibodies or the VL and VH of Table 19. In another embodiment, the one or more TM molecules of the TM are non-proteinaceous or are other small molecule receptor ligands. In some embodiments, the one or more non-proteinaceous TM are folate. In another embodiment, the one or more TM molecules are LHRH. In another embodiment of the targeted conjugate composition, the one or more cytotoxic payloads that are conjugated to cysteine residues of the fusion protein CCD are identical and are selected from the group of payloads of Table 15. In another embodiment of the targeted conjugate composition, the one or more cytotoxic payloads that are conjugated to cysteine residues of the fusion protein CCD are identical and are selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A. In one embodiment of the foregoing, the cytoxic payload is doxirubicin. In another embodiment of the foregoing, the cytotoxic payload is MMAE. In another embodiment of the foregoing, the cytotoxic payload is MMAF. In another embodiment of the foregoing, the cytotoxic payload is maytansine. In another embodiment of the foregoing, the cytotoxic payload is paclitaxel. In another embodiment of the foregoing, the cytotoxic payload is Pseudomonas exotoxin. In another embodiment of the foregoing, the cytotoxic payload is mertansine (DM1). In other embodiments, the targeted conjugate composition comprise two different cytotoxic drugs that are conjugated to the CCD cysteine residues of separate fusion proteins that are subsequently conjugated to the backbone XTEN, wherein each cytotoxic drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A.

In one embodiment of the targeted conjugate composition, the peptidic cleavage moiety (PCM) is selected from the group of sequences set forth in Table 8. In another embodiment of the targeted conjugate composition, the PCM of the composition is a substrate for protease associated with a tissue wherein an antigen, marker or receptor on said tissue is also a ligand for the TM of the composition. In such embodiments, the binding of the TM to the ligand brings the targeted conjugate composition bearing the cytotoxic drug or biologic into proximity with the tissue-associated protease whereupon the composition is cleaved, thereby releasing the components comprising the cytotoxic payloads proximal to the tissue such that the smaller molecular mass is capble of being internalized within said tissue, resulting in a pharmacologic effect know in the art for the cytoxic component. In one embodiment, the targeted conjugate composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a cell line comprising said tissue ligand compared to the toxicity of the composition when the cell line does not comprise said tissue ligand. In another embodiment, the composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a target tissue-associated protease present compared to the toxicity of the composition when the assay does not comprise said target tissue-associated protease. In another embodiment, the composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay wherein the PCM is cleaved compared to the toxicity of the composition when PCM is not cleaved. In another embodiment, the targeted conjugate composition TM-CCD fragment comprising the cytotoxic compound(s) (the released targeted composition) that is cleaved and released from the composition is internalized into a target cell in an in vitro mammalian cell cytotoxicity assay at a concentration that is least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater compared the intact composition. In another embodiment, the targeted conjugate composition exhibits a terminal half-life when administered to a subject that is 10-fold, or 20-fold, or 30-fold, or 40-fold, or 50-fold, or 100-fold longer compared to the corresponding cytotoxic drug not linked to the targeted conjugate composition and administered in a comparable fashion to a subject. In another embodiment, the targeted conjugate composition exhibits a terminal half-life of at least about 3 days, or at least about 7 days, or at least about 10 days, or at least about 14 days, or at least about 21 days, or at least about 30 days when administered to a subject. In another embodiment, the invention provides a targeted conjugate composition that when administered to a subject is cleaved by a protease colocalized with the target tissue, releasing the TM-CCD fragment comprising the cytotoxic compound(s) (the released targeted composition), and the released targeted composition is internalized into the target tissue bearing the ligand to a concentration that is least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater compared the intact composition. In another embodiment, the targeted conjugate composition exhibits a terminal half-life when administered to a subject that is 10-fold, or 20-fold, or 30-fold, or 40-fold, or 50-fold, or 100-fold longer compared to the corresponding cytotoxic drug not linked to the targeted conjugate composition and administered in a comparable fashion to a subject. In another embodiment, the targeted conjugate composition exhibits a terminal half-life of at least about 3 days, or at least about 7 days, or at least about 10 days, or at least about 14 days, or at least about 21 days, or at least about 30 days when administered to a subject.

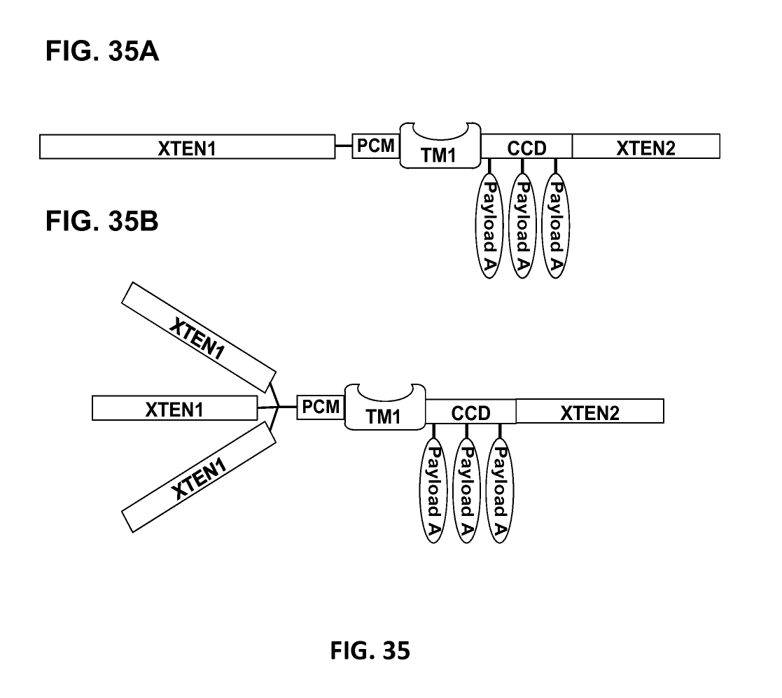

In another aspect, the invention provides targeted conjugate compositions comprising a first and a second region wherein each region is linked at its N-terminus to a peptidic cleavage moiety (PCM) that is a substrate for a protease associated with a tissue, with the PCM separating the composition into two regions; a first region in which a CCD fused to an unmodified XTEN that exhibits at least 90%, or 91%, or 92%, or 93%, or 94%, or 95%, or 96%, or 97%, or 98%, or 99% identity or is identical to a sequence selected from the XTEN sequences of Table 10, and a second region comprising a CCD fused to a second unmodified XTEN in which the XTEN exhibits at least 90%, or 91%, or 92%, or 93%, or 94%, or 95%, or 96%, or 97%, or 98%, or 99% identity or is identical to a sequence selected from the XTEN sequences of Table 10 wherein the second CCD further comprises one or more cytotoxic payloads that are conjugated to the cysteine residues of the second CCD, and wherein the composition further comprises one or more targeting moieties (TM) conjugated to cysteine residue(s) of the first CCD. Non-limiting examples of the resulting compositions are portrayed schematically in FIGS. 34 and 35. In one embodiment of the foregoing compositions of this paragraph, the TM conjugated to the PCM is a scFv derived from the group of antibodies or are scFv derived from the VL and VH of the antibodies of Table 19. In another embodiment of the foregoing, the TM conjugated to the second CCD is non-proteinaceous or are small molecule receptor ligands. In some embodiments, the one or more non-proteinaceous TM are folate. In yet another embodiment of the foregoing, the TM conjugated to the second CCD is an LHRH analog described herein. In one embodiment of the foregoing compositions, the peptidic cleavage moiety (PCM) is selected from the group of sequences set forth in Table 8. In another embodiment of the foregoing compositions, the PCM of the composition is a substrate for protease associated with a tissue wherein an antigen, marker or receptor on said tissue is also a ligand for the TM of the composition. In another embodiment of the foregoing compositions, the one or more cytotoxic payloads conjugated to the first CCD are identical and are selected from the group of payloads of Table 15. In another embodiment of the foregoing compositions of this paragraph, the one or more cytotoxic payloads conjugated to the first CCD are identical and are selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A. In another embodiment of the foregoing, the cytotoxic payload is MMAF. In another embodiment of the foregoing, the cytotoxic payload is maytansine. In another embodiment of the foregoing, the cytotoxic payload is paclitaxel. In another embodiment of the foregoing, the cytotoxic payload is Pseudomonas exotoxin. In another embodiment of the foregoing, the cytotoxic payload is MMAE. In other embodiments of the foregoing compositions of this paragraph, the composition comprises two different cytotoxic drugs selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A in which one drug is linked to the first CCD and the second drug is linked to the second CCD and the TM is fused to the terminal ends of the construct. In such embodiments, the binding of the TM to the ligand brings the composition into proximity with the tissue-associated protease whereupon the PCM of the composition is cleaved, thereby releasing the CCD comprising the cytotoxic payloads proximal to or that are internalized within said tissue, resulting in a pharmacologic effect know in the art for the cytoxic component. In this embodiment, the cleaved composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a cell line comprising said tissue ligand compared to the toxicity of the composition when the cell line does not comprise said tissue ligand. In another embodiment, this composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay with a cell line comprising said tissue ligand and in the presenece of the tissue-associated protease compared to the toxicity of the composition when cell line does not comprise said tissue ligand and the tissue-associated protease. Additionally, the composition exhibits at least about 2-fold, or 3-fold, or 4-fold, or 5-fold, or 6-fold, or 7-fold, or 8-fold, or 9-fold, or 10-fold, or 20-fold, or 30-fold, or 40-fold, or 50-fold, or 100-fold greater toxicity in an in vitro mammalian cell cytotoxicity assay wherein the PCM is cleaved compared to the toxicity of the composition when PCM is not cleaved. As a result of the presences of the XTEN fused to the composition, the composition exhibits a terminal half-life when administered to a subject that is 10-fold, or 20-fold, or 30-fold, or 40-fold, or 50-fold, or 100-fold longer compared to the corresponding cytotoxic drug not linked to the composition and administered in a comparable fashion to a subject. In the embodiment, the composition exhibits a terminal half-life of at least about 3 days, or at least about 7 days, or at least about 10 days, or at least about 14 days, or at least about 21 days, or at least about 30 days when administered to a subject.

In another aspect, the invention provides targeted conjugate compositions comprising at least one targeting moiety directed to a target selected from the group consisting of the targets set forth in Tables 2, 3, 4, 19 and 19 fused to the fusion proteins comprising a CCD, a PCM, and an XTEN wherein the composition further comprises one or more molecules of a cytotoxic payload conjugated to the cysteine residues of the CCD. In one embodiment of the composition, the TM is an scFV derived from the antibodies or the VL and VH sequences of Table 19. In another embodiment, the TM is folate, which is conjugated to the N- or C-terminus of the CCD In another embodiment, the TM is LHRH conjugated to the N- or C-terminus of the CCD. In the foregoing compositions, the cytotoxic payload molecules are identical and are selected from the group of payloads of Tables 14-17. In another embodiment of the foregoing compositions, the one or more cytotoxic payloads are identical and are selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A.

In other embodiments, the targeted conjugate compositions comprise two different cytotoxic drugs selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, and Pseudomonas exotoxin A in which each type of cytotoxic drug is conjugated to a different CCD of the fusion protein such that each CCD of a given composotion comprises only identical cytotoxic drugs.

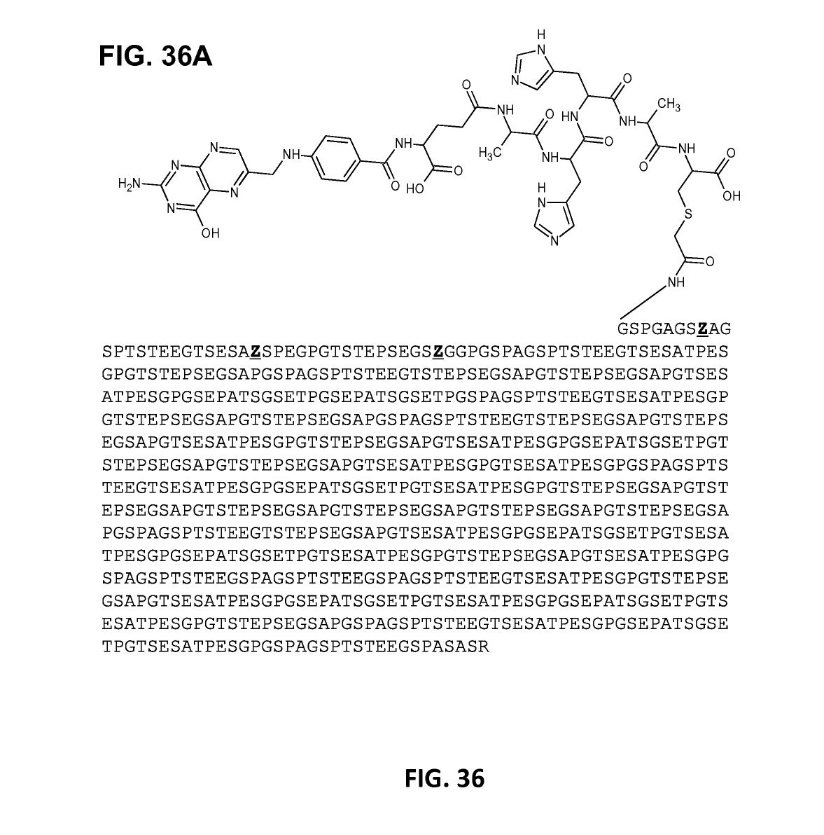

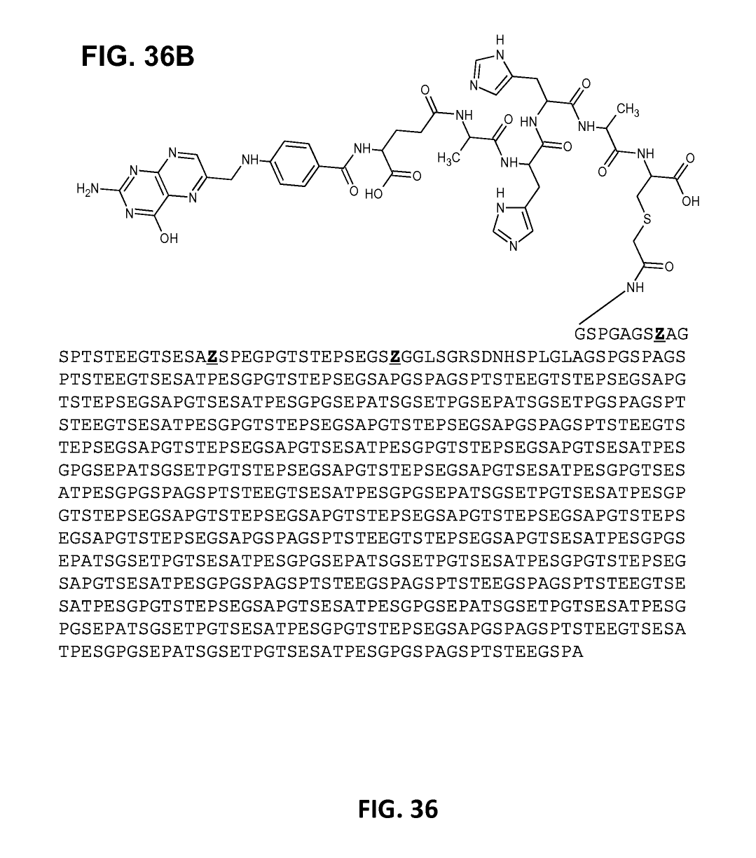

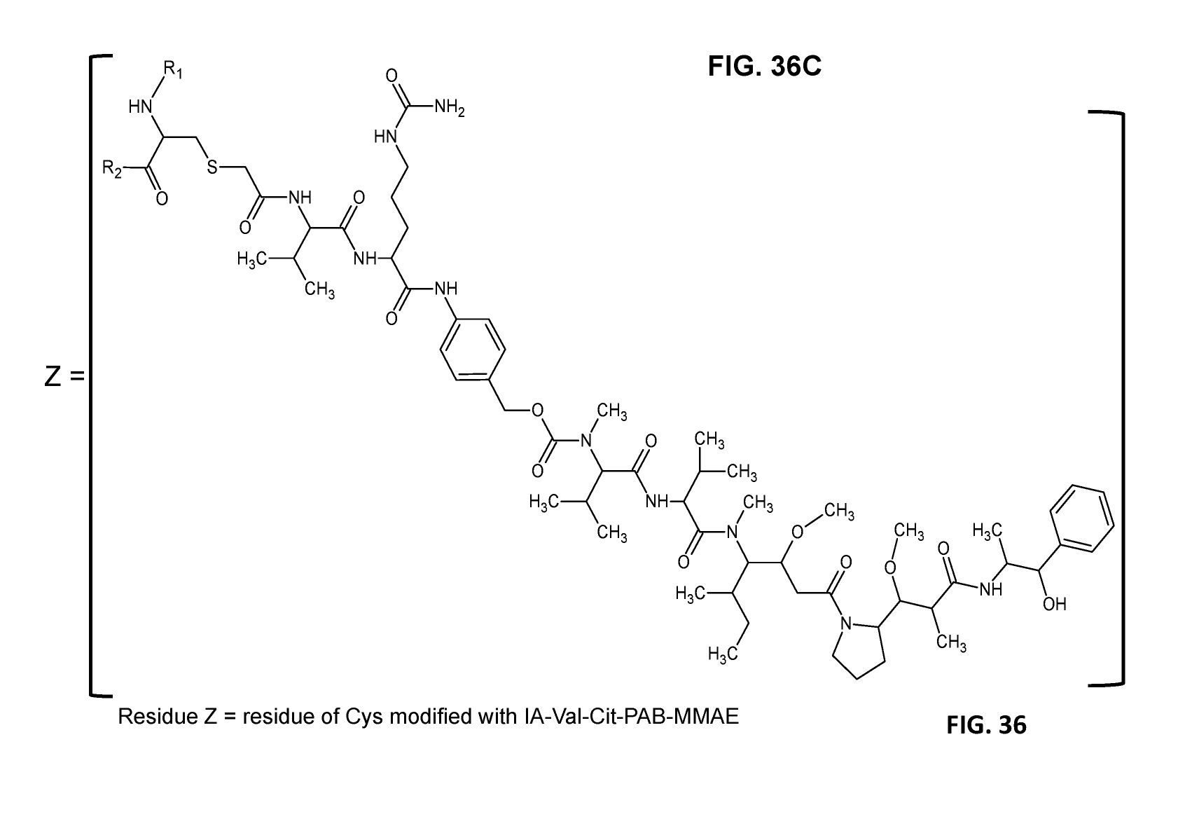

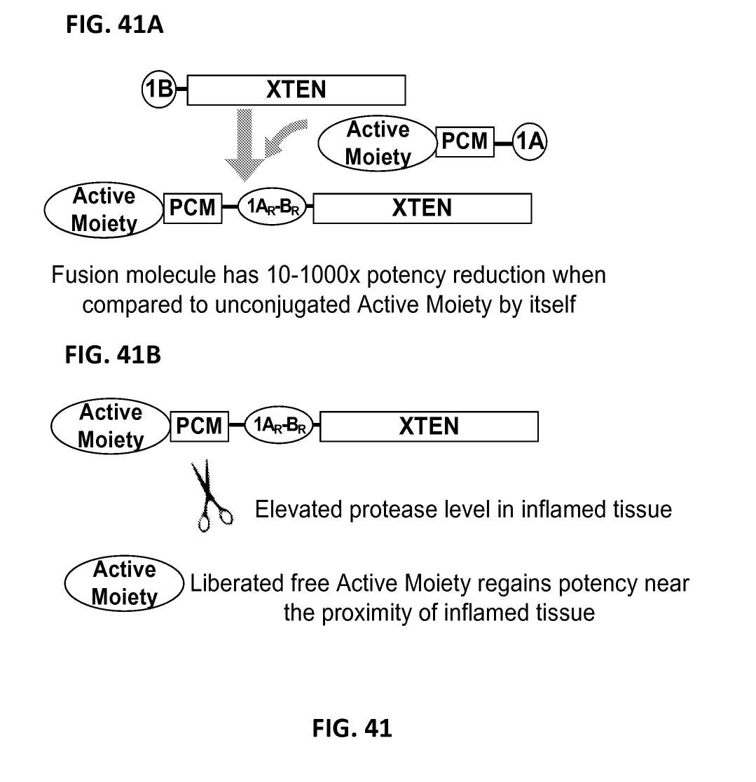

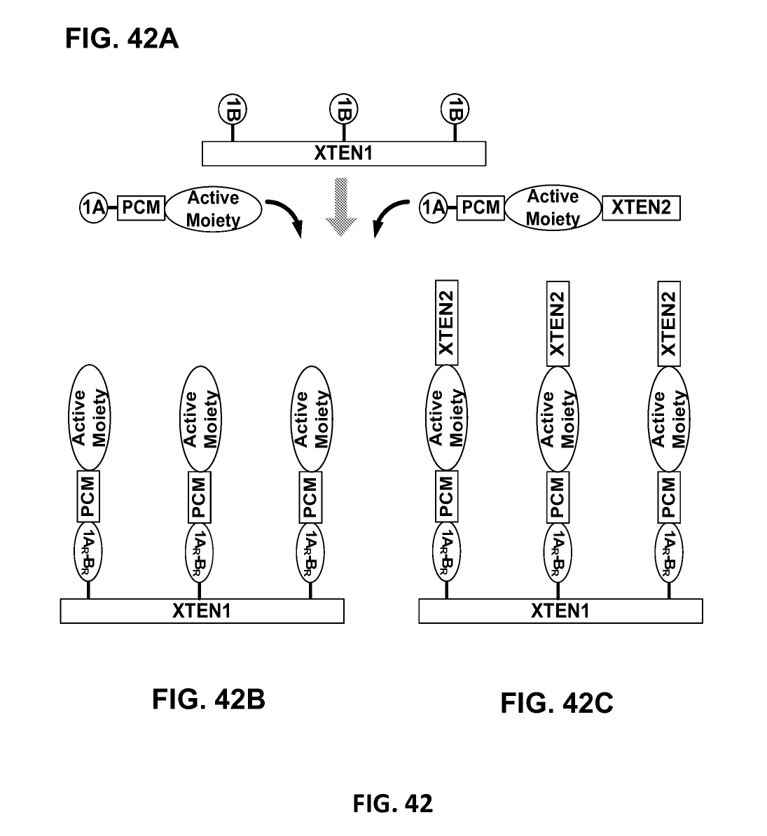

As illustrated in FIGS. 34-37, 41, 42 and 46-51, the subject targeted conjugate compositions can have different valencies, with one, two, three, or four or more fusion protein molecules linked to one or more targeting moieties. Accordingly, a targetedconjugate composition can comprise 1, 2, 3, or 4 or more fusion proteins comprising CCD with linked payloads and targeting moieties.

TABLE 1
Targeting Moieties: Antibody fragments, scaffolds and mimetics
Targeting Moieties

ABDURINS
AdNectins/Fibronectin type III domain
Adnexins/Fibronectin
Affibodies/Protein Z
Affilins
AFFINILUTE
AFFINIMIP
AFIM
Anticalins/Lipocalins
Aptabody
Aptamers
Armadillo repeat proteins
Avimers
Azymetric
Bispecific diabodies
BiTEs
Bivalent diabodies
Centyrins
DARPins/Ankyrin repeat proteins
Diabodies
Domain antibodies/dAbs/human Vh
Engineered affinity proteins
Evibodies
Fabs
Fv
Fynomers
Glubody
Im7/ColE7 immunity protein
iMabs
Knottin/Cysteine-knot miniproteins
Kunitz domains
Maxibodies
Microbodies
Minibodies
Molecular imprinted polymers (MIPs)
Monobodies
Monoclonal antibodies
Monoclonal T cell receptors (mTCR)
MonoLex
Nanobodies
Nanofitins
Phylomers
Single chain variable fragment (scFv)
Single chain Fab (scFab) fragment
Shark Vhh
SMIPs
SOMAmers
Stable scFV
Spiegelmers
Synbodies
TandAbs ®
Telobodies
Tetrabodies
Tetranectins
Tetravalent bispecific antibodies
Trans-body
Triabodies

TABLE 2
Exemplary targets and targeting
moieties to which conjugate compositions
can be directed

| Class | Target | Targeting Moiety |
|---|---|---|
| Peptide | LHRHR | LHRH & analogues (e.g. D-Lys-(6)-LHRH) |
| | CD13, Aminopeptidase | NGR class (e.g. CNGRC (SEQ ID NO: 5), CNGRCG |
| | | (SEQ ID NO: 6), GNGRG (SEQ ID NO: 7), KNGRE |
| | | (SEQ ID NO: 8), (GNGRG)2KGK (core peptide |
| | | disclosed as SEQ ID NO: 9), CVLNGRMEC (SEQ ID |
| | | NO: 10), NGRAHA (SEQ ID NO: 11), |
| | | CNGRCVSGCAGRC (SEQ ID NO: 12)) |
| | Folate receptor | Folate & analogue (e.g. γ-folate, α-folate; pteroate-gly) |
| | Integrin | Cilengitide; RGD-4C; iRGD |
| | LRP receptor | Angiopep-2 |
| | Somatostatin receptor | Somatostatin & analogues (e.g. octreotide; pasireotide; |
| | | lanreotide; vapreotide, JF-07-69) |
| | Nucleolin | F3 peptide |
| | PDGFR-beta | RGR |
| | LyP-1 receptor | LyP-1; CGNKRTRGC (SEQ ID NO: 13) |
| | Chondroitin sulfate | TAASGVRSMH (SEQ ID NO: 14); LTLRWVGLMS |
| | proteoglygan NG2 | (SEQ ID NO: 15) |
| | VPAC1 and VPAC2 | Vasoactive intestinal peptide |
| | CCK1 and CCK2 | Cholecystokinin |
| | Gastrin receptor, CCK1 & | Gastrin |
| | CCK2 | |
| | GRP receptor subtype | Gastrin-releasing peptide |
| | Neurotensin receptor | Neurotensin |
| | Alpha-MSH receptor | Alpha-melanocyte stimulating hormone |
| | Oxytocin receptor | Oxytocin |
| | Lymphatic vessels | LyP-2; CNRRTKAGC (SEQ ID NO: 16) |
| | Lymphatic vessels | LSD; CLSDGKRKC (SEQ ID NO: 17) |
| | Lymphatic vessels | REA; CREAGRKAC (SEQ ID NO: 18) |
| | Lymphatic vessels | AGR, CAGRRSAYC (SEQ ID NO: 19) |
| | Pericytes & endothelia | RSR; CRSRKG (SEQ ID NO: 20) |
| | cells | |
| | Pericytes & endothelia | KAA; CKAAKNK (SEQ ID NO: 21) |
| | cells | |
| | Blood vessels | CSRPRRSEC (SEQ ID NO: 22) |
| | Angiogenic blood vessels | KRK; CGKRK (SEQ ID NO: 23) |
| | & tumor cells | |
| | Angiogenic blood vessels | CDTRL (SEQ ID NO: 24) |
| | Angiogenic blood vessels | CGTKRKC (SEQ ID NO: 25) |
| | & tumor cells | |
| Protein | DR4, DR5 | TRAIL |
| Antibody- | Various | DARPINS |
| like scaffold | Various | Centyrins |
| Antibody | Lewis-Y-related antigen | Br96; anti-Lewis-Y-related antigen antibody |
| | Her2 | Trastuzumab; Pertuzumab; anti-HER2 antibody |
| | EGFR | Cetuximab; anti-EGFR antibody |
| | Nectin-4 | anti-nectin-4 antibody |
| | CanAg (mucin-type | huC242, anti-CanAg antibody |
| | glycoprotein) | |
| | CD138 | anti-CD138 antibody |
| | CD19 | MDX-1342; MOR-208; HuB4; anti-CD19 antibody |
| | CD22 | Epratuzumab; Bectumomab; Inotuzumab; |
| | | Moxetumomab, RFB4; anti-CD22 antibody |
| | CD23 | Lumiliximab, anti-CD23 antibody |
| | CD25 (IL-2 receptor) | Daclizumab, anti-CD25 antibody |
| | CD30 | Xmab2513; cAC10; MDX-060; anti-CD30 antibody |
| | CD33 | Gemtuzumab; HuM195; huMy9-6; anti-CD33 antibody |
| | CD38 | Daratumumab, anti-CD38 antibody |
| | CD40 | SGN-40; HCD122; anti-CD40 antibody |
| | CD56 | huN901; anti-CD56 antibody |
| | CD70 | MDX-1411; anti-CD70 antibody |
| | CD74 | Milatuzumab; anti-CD74 antibody |
| | CD79b | anti-CD79b antibody |
| | CD80 | Galiximab; anti-CD80 antibody |
| | Carcinoembryonic antigen | Lapetuzumab, hCOL-1anti-CEA antibody |
| | (CEA) | |
| | Cripto | anti-Cripto antibody |
| | cMET | CE-355621, DN30, MetMAb; antagonist anti-cMET antibody |
| | EpCAM | Adecatumumab; Edrecolomab; Catumaxomab; anti-EpCAM antibody |
| | EphA2 | 1C1, anti-EphA2 antibody |
| | GPNMB (human | glembatumumab, anti-GPNMB antibody |
| | gylcoprotein NMB | |
| | (osteoactivin)) | |
| | Integrins | anti-integrin antibody |
| | MUC-1 (epitope CA6) | anti-MUC-1 antibody |
| | PSMA | MDX-070, MLN591, anti-PSMA antibody |
| | TGFa | anti-TGFa antibody |
| | TIM1 | anti-TIM1 antibody |
| | Folate receptor 1 | M9346A, Farletuzumab, anti-folate receptor antibody |
| | IL-13 receptor | anti-IL-13 receptor antibody |

Additional targets contemplated for which the targeting moieties of the subject targeted conjugate compositions of the invention can be directed include tumor-associated antigens listed in Table 3. In one embodiment, the invention provides targeted conjugate compositions comprising one or more targeting components capable of binding one or more of the tumor associated antigens of Table 3 and the cancer target ligands of Table 2, Table 4, or Table 19.

TABLE 3
Tumor-associated antigen targets

| TAA targets (synonyms) | Accession Number and References |
|---|---|
| Her2 (ErbB2) | GenBank accession no. M11730; U.S. Pat. No. 5,869,445; |
| | WO2004048938; WO2004027049; WO2004009622; |
| | WO2003081210; WO2003089904; WO2003016475; |
| | US2003118592; WO2003008537; WO2003055439; |
| | WO2003025228; WO200222636; WO200212341; |
| | WO200213847; WO200214503; WO200153463; |
| | WO200141787; WO200044899; WO200020579; |
| | WO9630514; EP1439393; WO2004043361; |
| | WO2004022709; WO200100244 |
| BMPR1B (bone morphogenetic | GenBank accession no. NM_001203; WO2004063362; |
| protein receptor-type IB) | WO2003042661; US 2003134790; WO2002102235; |
| | WO2003055443; WO200299122; WO2003029421; |
| | WO2003024392; WO200298358' WO200254940; |
| | WO200259377; WO200230268 |
| E16 (LAT1, SLC7A5) | GenBank accession no. NM_003486); WO2004048938; |
| | WO2004032842; WO2003042661; WO2003016475; |
| | WO200278524; WO200299074; WO200286443; |
| | WO2003003906; WO200264798; WO200014228; |
| | US2003224454; WO2003025138 |
| STEAP1 (six transmembrane | GenBank accession no. NM_012449; WO2004065577; |
| epithelial antigen of prostate) | WO2004027049; EP1394274; WO2004016225; |
| | WO2003042661; US2003157089; US2003185830; |
| | US2003064397; WO200289747618; WO2003022995 |
| STEAP2 (six transmembrane | GenBank accession no. AF455138; WO2003087306; |
| epithelial antigen of prostate 2) | US2003064397; WO200272596; WO200172962; |
| | WO2003104270; WO2003104270; US2004005598; |
| | WO2003042661; US2003060612; WO200226822; |
| | WO200216429 |
| CA125/0772P (MUC16) | GenBank accession no. AF361486; WO2004045553; |
| | WO200292836; WO200283866; US2003124140 |
| megakaryocyte potentiating factor | GenBank accession no. NM_005823; WO2003101283; |
| (MPF, mesothelin) | WO2002102235; WO2002101075; WO200271928; |
| | WO9410312 |
| Na/Pi cotransporter type IIb (NaPi3b) | GenBank accession no. NM_006424; WO2004022778; |
| | EP1394274; WO2002102235; EP875569; WO200157188; |
| | WO2004032842; WO200175177 |
| Semaphorin 5b (SEMA5B, SEMAG) | GenBank accession no. AB040878; WO2004000997; |
| | WO2003003984; WO200206339; WO200188133; |
| | WO2003054152; WO2003101400 |
| Prostate cancer stem cell antigen | GenBank accession no. AY358628; US2003129192; |
| (PSCA hlg) | US2004044180; US2004044179; US2003096961; |
| | US2003232056; WO2003105758; US2003206918; |
| | EP1347046; WO2003025148 |
| ETBR (Endothelin type B receptor) | GenBank accession no. AY275463; WO2004045516; |
| | WO2004048938; WO2004040000; WO2003087768; |
| | WO2003016475; WO2003016475; WO200261087; |
| | WO2003016494; WO2003025138; WO200198351; |
| | EP522868; WO200177172; US2003109676; U.S. Pat. No. |
| | 6,518,404; U.S. Pat. No. 5,773,223; WO2004001004 |
| TRPV4 (Transient receptor potential | U.S. patent application No. 20090208514 |
| cation channel, subfamily V) | |
| CDC45L | GenBank Accession NO. AJ223728; U.S. patent application No. |
| | 20090208514 |
| CRIPTO (CR, CR1, CRGF) | GenBank accession no. NP_003203 or NM_003212; |
| | US2003224411; WO2003083041; WO2003034984; |
| | WO200288170; WO2003024392; WO200216413; |
| | WO200222808; U.S. Pat. No. 5,854,399; U.S. Pat. No. 5,792,616 |
| CD21 (CR2 (Complement receptor 2) | GenBank accession no. M26004; WO2004045520; |
| or C3DR (C3d/Epstein Barr virus | US2004005538; WO2003062401; WO2004045520; |
| receptor) | WO9102536; WO2004020595 |
| CD79b (CD79B, CD79β, IGb | GenBank accession no. NM_000626 or 11038674; |
| (immunoglobulin-associated beta), | WO2004016225; WO2003087768; US2004101874; |
| B29) | WO2003062401; WO200278524; US2002150573; U.S. Pat. |
| | No. 5,644,033; WO2003048202; WO 99/558658, U.S. Pat. |
| | No. 6,534,482; WO200055351 |
| FcRH2 (IFGP4, IRTA4, SPAP1A | GenBank accession no. NM_030764, AY358130; |
| (SH2 domain containing phosphatase | WO2004016225; WO2003077836; WO200138490; |
| anchor protein 1a), SPAP1B, | WO2003097803; WO2003089624 |
| SPAP1C) | |
| NCA (CEACAM6) | GenBank accession no. M18728; WO2004063709; |
| | EP1439393; WO2004044178; WO2004031238; |
| | WO2003042661; WO200278524; WO200286443; |
| | WO200260317 |
| MDP (DPEP1) | GenBank accession no. BC017023; WO2003016475; |
| | WO200264798 |
| IL20Rα (IL20Ra, ZCYTOR7) | GenBank accession no. AF184971; EP1394274; |
| | US2004005320; WO2003029262; WO2003002717; |
| | WO200222153; US2002042366; WO200146261; |
| | WO200146232; WO9837193 |
| BECAN (Brevican core protein) | GenBank accession no. AF229053; US2003186372; |
| | US2003186373; US2003119131; US2003119122; |
| | US2003119126; US2003119121; US2003119129; |
| | US2003119130; US2003119128; US2003119125; |
| | WO2003016475; WO200202634 |
| EphB2R (DRT, ERK, Hek5, EPHT3, | GenBank accession no. NM_004442; WO2003042661; |
| TyroS) | WO200053216; WO2004065576 (Claim 1); |
| | WO2004020583; WO2003004529; WO200053216 |
| B7h (ASLG659) | GenBank accession no. AX092328; US20040101899; |
| | WO2003104399; WO2004000221; US2003165504; |
| | US2003124140; US2003065143; WO2002102235; |
| | US2003091580; WO200210187; WO200194641; |
| | WO200202624; US2002034749; WO200206317; |
| | WO200271928; WO200202587; WO200140269; |
| | WO200036107; WO2004053079; WO2003004989; |
| | WO200271928 |
| PSCA (Prostate stem cell antigen | GenBank accession no. AJ297436; WO2004022709; |
| precursor | EP1394274; US2004018553; WO2003008537 (Claim 1); |
| | WO200281646; WO2003003906; WO200140309; |
| | US2001055751; WO200032752; WO9851805; |
| | WO9851824; WO9840403 |
| BAFF-R (B cell-activating factor | GenBank accession No. AF116456; WO2004058309; |
| receptor, BLyS receptor 3, BR3) | WO2004011611; WO2003045422; WO2003014294; |
| | WO2003035846; WO200294852; WO200238766; |
| | WO200224909 |
| CD22 (B-cell receptor CD22-β-form, | GenBank accession No. AK026467; WO2003072036 |
| BL-CAM, Lyb-8, Lyb8, SIGLEC-2, | |
| FLJ22814) | |
| CD79a (immunoglobulin-associated | GenBank accession No. NP_001774.10; WO2003088808, |
| alpha) | US20030228319; WO2003062401; US2002150573; |
| | WO9958658; WO9207574; U.S. Pat. No. 5,644,033 |
| CXCR5 (Burkitt's lymphoma receptor | GenBank accession No. NP_001707.1; WO2004040000; |
| 1) | WO2004015426; US2003105292; U.S. Pat. No. 6,555,339; |
| | WO200261087; WO200157188; WO200172830; |
| | WO200022129; WO9928468; U.S. Pat. No. 5,440,021; |
| | WO9428931; WO9217497 |
| HLA-DOB | GenBank accession No. NP_002111.1; WO9958658; U.S. |
| | Pat. No. 6,153,408; U.S. Pat. No. 5,976,551; U.S. Pat. No. |
| | 6,011,146 |
| P2X5 | GenBank accession No. NP_002552.2; WO2004047749; |
| | WO2003072035; WO200222660; WO2003093444; |
| | WO2003087768; WO2003029277 |
| CD72 (B-cell differentiation antigen | GenBank accession No. NP_001773.1; WO2004042346; |
| CD72, Lyb-2) | WO2003026493; WO200075655 |
| CD180 (LY64) | GenBank accession No. NP_005573.1; US2002193567; |
| | WO9707198; WO2003083047; WO9744452 |
| FcRH1 (Fc receptor-like protein 1) | GenBank accession No. NP_443170.1) WO2003077836; |
| | WO200138490; WO2003089624; EP1347046; |
| | WO2003089624 |
| IRTA2 (Immunoglobulin superfamily | GenBank accession No. Human: AF343662, AF343663, |
| receptor translocation associated 2) | AF343664, AF343665, AF369794, AF397453; |
| | WO2003024392; WO2003077836; WO200138490 |
| TENB2 (TMEFF2, tomoregulin, | GenBank accession No. AF179274; AY358907, CAF85723, |
| TPEF, HPP1) | CQ782436; WO2004074320; WO2003042661; |
| | WO2003009814; EP1295944; WO200230268; |
| | WO200190304; US2004249130; US2004022727; |
| | WO2004063355; US2004197325; US2003232350; |
| | US2004005563; US2003124579 |
| CS1 (CRACC, 19A, APEX-1, | GenBank Accession No. NM 021181; US 20100168397 |
| FOAP12) | |
| DLL4 | GenBank Accession No. NM 019074; US 20100303812 |
| Lewis Y | ADB235860; U.S. Pat. No. 7,879,983 |
| CD40 (Bp50, CDW40, MGC9013, | AL035662.65; U.S. Pat. No. 6,946,129 |
| TNFRSF5, p50) | |
| OBA1 (5T4) | GenBank Accession No. NP_001159864.1; US |
| | 20100021483 |
| p97 | Woodbury et al., 1980, Proc. Natl. Acad. Sci. USA 77: |
| | 2183-2186; Brown et al., 1981, J. Immunol. 127: 539-546 |
| carcinoembryonic antigen (CEA) | GenBank Accession No. NP_004354.2; U.S. Pat. No. 6,676,924 |
| TAG-72 | U.S. Pat. No. 7,256,004 |
| DNA | |
| Neuropilin-1 (NRP1) | GenBank Accession No. NP_001019799.1; US |
| | 20080213268 |
| A33 | GenBank Accession No. NP_005805.1; U.S. Pat. No. 7,579,187 |
| Mucin-1 (MUC1) | GenBank Accession No. NP_001018016.1; |
| | NP_001018017.1; U.S. Pat. No. 7,183,388 |
| ED-B fibronectin | U.S. Pat. No. 7,785,591 |
| Thomsen-Friedenreich antigen (TF) | U.S. Pat. No. 7,374,755; US 20100297159 |
| Bombesin receptor | U.S. Pat. No. 5,750,370 |
| CanAg | |
| Carcinoembryonic antigen (CEA) | U.S. Pat. No. 4,818,709 |
| CD13 | |
| CD138 | |
| CD30 | |
| CD33 | |
| CD38 | |
| CD40 | |
| CD47 | |
| CD56 | |
| CD70 | |
| Chondroitin sulfate proteoglygan NG2 | |
| EphA2 | |
| Folate receptor 1 | U.S. Pat. No. 5,547,668 |
| gastrin receptor | |
| GPNMB (human gylcoprotein) | |
| NMB (osteoactivin) | |
| GRP receptor subtype | |
| integrin avb3 | US20110166072 |
| LHRHR | US20110104074 |
| LRP receptor | |
| LyP-1 receptor | |
| Nectin-4 | |
| Neurotensin receptor | U.S. Pat. No. 8,058,230 |
| Nucleolin | |
| Somatostatin receptor | |
| TIM1 | |
| VPAC1 | |
| VPAC2 | |
| Alpha-MSH receptor | |
| CD25 | |
| Interleukin-1 receptor | |

TABLE 4
Target Ligands for Targeting Moieties
Cancer Target Ligand

Androgen Receptor
Alpha integrins
Alpha-V Beta 6 integrin
angiopoietin 2
B7-H3
BAFF
C242 antigen
CA-125
carbonic anhydrase 9 (CA-IX)
cardiac myosin
CCR4
CD117
CD11a
CD13
CD132
CD137
CD138
CD140
CD152
CD166
CD172A
CD19
CD2
CD20
CD22
CD221
CD23
CD25
CD28
CD3
CD30
CD33
CD37
CD38
CD40
CD40L
CD41
CD44
CD47
CD51
CD52
CD56
CD6
CD64
CD70
CD74
CD80
CD81
CD86
CD9
CD95
CEACAM5
CEACAM6
Claudin-3
Claudin-4
cMet
CSFR
CSFR-1
CTLA-4
DLL4
DPP4
EGFR
EpCAM
EphA2
FAP
FGF2
FGF4
FGF8
FGFR1
FGFR2
FGFR3
FGFR4
Folate Receptor
G-CSF
GLUT1
GLUT4
GM-CSF
GP130
GPNMB
HE4
HER2/neu, CD3
HER3
HGF
HLA-DR
human scatter factor receptor kinase
IGF-1 receptor
IGF1R
IGF-I
IgG4
IL1
IL11
IL12
IL12R
IL13
IL-13
IL13R
IL15
IL-17A
IL18
IL1R
IL2
IL21
IL23
IL23R
IL27
IL27R
IL29
IL2R
IL31
IL31R
IL4
IL4R
IL6
IL-6
IL6R
ILGF2
INSR
integrin α5β1
integrin αvβ3
Jagged Receptors and Ligands
LAG-3
Lewis-Y antigen
LIFR
LTA
MCP-1
mesothelin
MRP4
MS4A1
MUC1
mucin CanAg
NARP-1
Neutrophil Elastase
NGF
N-glycolylneuraminic acid
Nicastrin
Notch Ligands
Notch Receptors
OX40
PCSK9
PD-1
PDGF Ligands
PDGF Receptors
PDGF-R α
PD-L1
PD-L2
PSCA
PSMA
Rhesus factor
RON
SDC1
SLAMF7
sphingosine-1-phosphate
TAG-72
TEM1
tenascin C
TGF beta
TIM-3
TLR2-9
TNF alpha
TNFR
TRAIL receptors
TRAIL-R1
TRAIL-R2
tumor antigen CTAA16.88
tumor specific glycosylation of MUC1
TWEAK receptor
TYRP1(glycoprotein 75)
VAM-1
VEGF
VEGF Receptors
VEGF-A
VEGFR1
VEGFR2
vimentin

In particular embodiments, the invention provides targeted conjugate compositions comprising one, two or more targeting moieties and one, two or more types of drugs conjugated to different CCD, and one, two or more XTEN. Non-limiting embodiments of specific targeted conjugate compositions are provided in Table 5, in which the named composition has specified components of: i) targeting moiety; ii) CCD; iii) PCM sequence; iv) XTEN sequences of Table 10 and v) drug (wherein a drug molecule is linked to each cysteine of the corresponding CCD). With respect to the XTEN sequences of Table 5 in the listed conjugates, it is specifically intended that the XTEN can encompass the AE, AF and AG variations of the XTEN described in Table 10; e.g., XTEN864 includes AE864, AF864 and AG864. In one embodiment, a targeted conjugate compositiona of Table 5 is configured according to formula II, below. In another embodiment, a targeted conjugate composition of Table 5 is configured according to formula III, below. As would be appreciated by one of skill in the art, it is specifically contemplated that other combinations of the disclosed components, as well as different numbers or ratios of the respective specified components, as well as different XTEN sequences to which the payloads are conjugated, as well as different targeting moieties described herein may be substituted for those indicated in the exemplary examples in the Table. For example, the invention contemplates that the number of drug molecules attached to a given CCD can be 1, or 2, or 3, or 4, or 5, or 6, or 7, or 8, or 9 or more and that the CCD would have, the corresponding number of cysteine residues to which the drug moieties would be conjugated. Further, the invention contemplates that the number of targeting moieties attached to the subject compositions can be 1, or 2, or 3 or more, which would similarly be fused to an N-terminal amino group or conjugated to a corresponding number of cysteine or lysine residues in the composition.

TABLE 5
Exemplary targeted conjugate compositions

| | Conjugate Description* | | Conjugate Description |
|---|---|---|---|
| Conjugate | (TM¹ + CCD² + PCM³ + XTEN⁴ + Drug⁵) | Conjugate | (TM¹ + CCD² + PCM³ + XTEN⁴ + Drug⁵) |
| 1 | aHER2_scFv-CCD50-BSRS1-XTEN713- | 433 | aHER2_scFv-CCD50-BSRS1-XTEN713- |
| | DM1 | | MMAE |
| 2 | Folate-CCD50-BSRS1-XTEN713-DM1 | 434 | Folate-CCD50-BSRS1-XTEN713-MMAE |
| 3 | aCEA_scFv-CCD50-BSRS1-XTEN713- | 435 | aCEA_scFv-CCD50-BSRS1-XTEN713- |
| | DM1 | | MMAE |
| 4 | aEpCAM_scFv-CCD50-BSRS1-XTEN713- | 436 | aEpCAM_scFv-CCD50-BSRS1-XTEN713- |
| | DM1 | | MMAE |
| 5 | aHER2_scFv-CCD51-BSRS1-XTEN713- | 437 | aHER2_scFv-CCD51-BSRS1-XTEN713- |
| | DM1 | | MMAE |
| 6 | Folate-CCD51-BSRS1-XTEN713-DM1 | 438 | Folate-CCD51-BSRS1-XTEN713-MMAE |
| 7 | aCEA_scFv-CCD51-BSRS1-XTEN713- | 439 | aCEA_scFv-CCD51-BSRS1-XTEN713- |
| | DM1 | | MMAE |
| 8 | aEpCAM_scFv-CCD51-BSRS1-XTEN713- | 440 | aEpCAM_scFv-CCD51-BSRS1-XTEN713- |
| | DM1 | | MMAE |
| 9 | aHER2_scFv-CCD1-BSRS1-XTEN713- | 441 | aHER2_scFv-CCD1-BSRS1-XTEN713- |
| | DM1 | | MMAE |
| 10 | Folate-CCD1-BSRS1-XTEN713-DM1 | 442 | Folate-CCD1-BSRS1-XTEN713-MMAE |
| 11 | aCEA_scFv-CCD1-BSRS1-XTEN713- | 443 | aCEA_scFv-CCD1-BSRS1-XTEN713-MMAE |
| | DM1 | | |
| 12 | aEpCAM_scFv-CCD1-BSRS1-XTEN713- | 444 | aEpCAM_scFv-CCD1-BSRS1-XTEN713- |
| | DM1 | | MMAE |
| 13 | aHER2_scFv-CCD7-BSRS1-XTEN713- | 445 | aHER2_scFv-CCD7-BSRS1-XTEN713- |
| | DM1 | | MMAE |
| 14 | Folate-CCD7-BSRS1-XTEN713-DM1 | 446 | Folate-CCD7-BSRS1-XTEN713-MMAE |
| 15 | aCEA_scFv-CCD7-BSRS1-XTEN713- | 447 | aCEA_scFv-CCD7-BSRS1-XTEN713-MMAE |
| | DM1 | | |
| 16 | aEpCAM_scFv-CCD7-BSRS1-XTEN713- | 448 | aEpCAM_scFv-CCD7-BSRS1-XTEN713- |
| | DM1 | | MMAE |
| 17 | aHER2_scFv-CCD12-BSRS1-XTEN713- | 449 | aHER2_scFv-CCD12-BSRS1-XTEN713- |
| | DM1 | | MMAE |
| 18 | Folate-CCD12-BSRS1-XTEN713-DM1 | 450 | Folate-CCD12-BSRS1-XTEN713-MMAE |
| 19 | aCEA_scFv-CCD12-BSRS1-XTEN713- | 451 | aCEA_scFv-CCD12-BSRS1-XTEN713- |
| | DM1 | | MMAE |
| 20 | aEpCAM_scFv-CCD12-BSRS1-XTEN713- | 452 | aEpCAM_scFv-CCD12-BSRS1-XTEN713- |
| | DM1 | | MMAE |
| 21 | aHER2_scFv-CCD16-BSRS1-XTEN713- | 453 | aHER2_scFv-CCD16-BSRS1-XTEN713- |
| | DM1 | | MMAE |
| 22 | Folate-CCD16-BSRS1-XTEN713-DM1 | 454 | Folate-CCD16-BSRS1-XTEN713-MMAE |
| 23 | aCEA_scFv-CCD16-BSRS1-XTEN713- | 455 | aCEA_scFv-CCD16-BSRS1-XTEN713- |
| | DM1 | | MMAE |
| 24 | aEpCAM_scFv-CCD16-BSRS1-XTEN713- | 456 | aEpCAM_scFv-CCD16-BSRS1-XTEN713- |
| | DM1 | | MMAE |
| 25 | aHER2_scFv-CCD50-BSRS2-XTEN713- | 457 | aHER2_scFv-CCD50-BSRS2-XTEN713- |
| | DM1 | | MMAE |
| 26 | Folate-CCD50-BSRS2-XTEN713-DM1 | 458 | Folate-CCD50-BSRS2-XTEN713-MMAE |
| 27 | aCEA_scFv-CCD50-BSRS2-XTEN713- | 459 | aCEA_scFv-CCD50-BSRS2-XTEN713- |
| | DM1 | | MMAE |
| 28 | aEpCAM_scFv-CCD50-BSRS2-XTEN713- | 460 | aEpCAM_scFv-CCD50-BSRS2-XTEN713- |
| | DM1 | | MMAE |
| 29 | aHER2_scFv-CCD51-BSRS2-XTEN713- | 461 | aHER2_scFv-CCD51-BSRS2-XTEN713- |
| | DM1 | | MMAE |
| 30 | Folate-CCD51-BSRS2-XTEN713-DM1 | 462 | Folate-CCD51-BSRS2-XTEN713-MMAE |
| 31 | aCEA_scFv-CCD51-BSRS2-XTEN713- | 463 | aCEA_scFv-CCD51-BSRS2-XTEN713- |
| | DM1 | | MMAE |
| 32 | aEpCAM_scFv-CCD51-BSRS2-XTEN713- | 464 | aEpCAM_scFv-CCD51-BSRS2-XTEN713- |
| | DM1 | | MMAE |
| 33 | aHER2_scFv-CCD1-BSRS2-XTEN713- | 465 | aHER2_scFv-CCD1-BSRS2-XTEN713- |
| | DM1 | | MMAE |
| 34 | Folate-CCD1-BSRS2-XTEN713-DM1 | 466 | Folate-CCD1-BSRS2-XTEN713-MMAE |
| 35 | aCEA_scFv-CCD1-BSRS2-XTEN713- | 467 | aCEA_scFv-CCD1-BSRS2-XTEN713-MMAE |
| | DM1 | | |
| 36 | aEpCAM_scFv-CCD1-BSRS2-XTEN713- | 468 | aEpCAM_scFv-CCD1-BSRS2-XTEN713- |
| | DM1 | | MMAE |
| 37 | aHER2_scFv-CCD7-BSRS2-XTEN713- | 469 | aHER2_scFv-CCD7-BSRS2-XTEN713- |
| | DM1 | | MMAE |
| 38 | Folate-CCD7-BSRS2-XTEN713-DM1 | 470 | Folate-CCD7-BSRS2-XTEN713-MMAE |
| 39 | aCEA_scFv-CCD7-BSRS2-XTEN713- | 471 | aCEA_scFv-CCD7-BSRS2-XTEN713-MMAE |
| | DM1 | | |
| 40 | aEpCAM_scFv-CCD7-BSRS2-XTEN713- | 472 | aEpCAM_scFv-CCD7-BSRS2-XTEN713- |
| | DM1 | | MMAE |
| 41 | aHER2_scFv-CCD12-BSRS2-XTEN713- | 473 | aHER2_scFv-CCD12-BSRS2-XTEN713- |
| | DM1 | | MMAE |
| 42 | Folate-CCD12-BSRS2-XTEN713-DM1 | 474 | Folate-CCD12-BSRS2-XTEN713-MMAE |
| 43 | aCEA_scFv-CCD12-BSRS2-XTEN713- | 475 | aCEA_scFv-CCD12-BSRS2-XTEN713- |
| | DM1 | | MMAE |
| 44 | aEpCAM_scFv-CCD12-BSRS2-XTEN713- | 476 | aEpCAM_scFv-CCD12-BSRS2-XTEN713- |
| | DM1 | | MMAE |
| 45 | aHER2_scFv-CCD16-BSRS2-XTEN713- | 477 | aHER2_scFv-CCD16-BSRS2-XTEN713- |
| | DM1 | | MMAE |
| 46 | Folate-CCD16-BSRS2-XTEN713-DM1 | 478 | Folate-CCD16-BSRS2-XTEN713-MMAE |
| 47 | aCEA_scFv-CCD16-BSRS2-XTEN713- | 479 | aCEA_scFv-CCD16-BSRS2-XTEN713- |
| | DM1 | | MMAE |
| 48 | aEpCAM_scFv-CCD16-BSRS2-XTEN713- | 480 | aEpCAM_scFv-CCD16-BSRS2-XTEN713- |
| | DM1 | | MMAE |
| 49 | aHER2_scFv-CCD50-BSRS3-XTEN713- | 481 | aHER2_scFv-CCD50-BSRS3-XTEN713- |
| | DM1 | | MMAE |
| 50 | Folate-CCD50-BSRS3-XTEN713-DM1 | 482 | Folate-CCD50-BSRS3-XTEN713-MMAE |
| 51 | aCEA_scFv-CCD50-BSRS3-XTEN713- | 483 | aCEA_scFv-CCD50-BSRS3-XTEN713- |
| | DM1 | | MMAE |
| 52 | aEpCAM_scFv-CCD50-BSRS3-XTEN713- | 484 | aEpCAM_scFv-CCD50-BSRS3-XTEN713- |
| | DM1 | | MMAE |
| 53 | aHER2_scFv-CCD51-BSRS3-XTEN713- | 485 | aHER2_scFv-CCD51-BSRS3-XTEN713- |
| | DM1 | | MMAE |
| 54 | Folate-CCD51-BSRS3-XTEN713-DM1 | 486 | Folate-CCD51-BSRS3-XTEN713-MMAE |
| 55 | aCEA_scFv-CCD51-BSRS3-XTEN713- | 487 | aCEA_scFv-CCD51-BSRS3-XTEN713- |
| | DM1 | | MMAE |
| 56 | aEpCAM_scFv-CCD51-BSRS3-XTEN713- | 488 | aEpCAM_scFv-CCD51-BSRS3-XTEN713- |
| | DM1 | | MMAE |
| 57 | aHER2_scFv-CCD1-BSRS3-XTEN713- | 489 | aHER2_scFv-CCD1-BSRS3-XTEN713- |
| | DM1 | | MMAE |
| 58 | Folate-CCD1-BSRS3-XTEN713-DM1 | 490 | Folate-CCD1-BSRS3-XTEN713-MMAE |
| 59 | aCEA_scFv-CCD1-BSRS3-XTEN713- | 491 | aCEA_scFv-CCD1-BSRS3-XTEN713-MMAE |
| | DM1 | | |
| 60 | aEpCAM_scFv-CCD1-BSRS3-XTEN713- | 492 | aEpCAM_scFv-CCD1-BSRS3-XTEN713- |
| | DM1 | | MMAE |
| 61 | aHER2_scFv-CCD7-BSRS3-XTEN713- | 493 | aHER2_scFv-CCD7-BSRS3-XTEN713- |
| | DM1 | | MMAE |
| 62 | Folate-CCD7-BSRS3-XTEN713-DM1 | 494 | Folate-CCD7-BSRS3-XTEN713-MMAE |
| 63 | aCEA_scFv-CCD7-BSRS3-XTEN713- | 495 | aCEA_scFv-CCD7-BSRS3-XTEN713-MMAE |
| | DM1 | | |
| 64 | aEpCAM_scFv-CCD7-BSRS3-XTEN713- | 496 | aEpCAM_scFv-CCD7-BSRS3-XTEN713- |
| | DM1 | | MMAE |
| 65 | aHER2_scFv-CCD12-BSRS3-XTEN713- | 497 | aHER2_scFv-CCD12-BSRS3-XTEN713- |
| | DM1 | | MMAE |
| 66 | Folate-CCD12-BSRS3-XTEN713-DM1 | 498 | Folate-CCD12-BSRS3-XTEN713-MMAE |
| 67 | aCEA_scFv-CCD12-BSRS3-XTEN713- | 499 | aCEA_scFv-CCD12-BSRS3-XTEN713- |
| | DM1 | | MMAE |
| 68 | aEpCAM_scFv-CCD12-BSRS3-XTEN713- | 500 | aEpCAM_scFv-CCD12-BSRS3-XTEN713- |
| | DM1 | | MMAE |
| 69 | aHER2_scFv-CCD16-BSRS3-XTEN713- | 501 | aHER2_scFv-CCD16-BSRS3-XTEN713- |
| | DM1 | | MMAE |
| 70 | Folate-CCD16-BSRS3-XTEN713-DM1 | 502 | Folate-CCD16-BSRS3-XTEN713-MMAE |
| 71 | aCEA_scFv-CCD16-BSRS3-XTEN713- | 503 | aCEA_scFv-CCD16-BSRS3-XTEN713- |
| | DM1 | | MMAE |
| 72 | aEpCAM_scFv-CCD16-BSRS3-XTEN713- | 504 | aEpCAM_scFv-CCD16-BSRS3-XTEN713- |
| | DM1 | | MMAE |
| 73 | aHER2_scFv-CCD50-BSRS4-XTEN713- | 505 | aHER2_scFv-CCD50-BSRS4-XTEN713- |
| | DM1 | | MMAE |
| 74 | Folate-CCD50-BSRS4-XTEN713-DM1 | 506 | Folate-CCD50-BSRS4-XTEN713-MMAE |
| 75 | aCEA_scFv-CCD50-BSRS4-XTEN713- | 507 | aCEA_scFv-CCD50-BSRS4-XTEN713- |
| | DM1 | | MMAE |
| 76 | aEpCAM_scFv-CCD50-BSRS4-XTEN713- | 508 | aEpCAM_scFv-CCD50-BSRS4-XTEN713- |
| | DM1 | | MMAE |
| 77 | aHER2_scFv-CCD51-BSRS4-XTEN713- | 509 | aHER2_scFv-CCD51-BSRS4-XTEN713- |
| | DM1 | | MMAE |
| 78 | Folate-CCD51-BSRS4-XTEN713-DM1 | 510 | Folate-CCD51-BSRS4-XTEN713-MMAE |
| 79 | aCEA_scFv-CCD51-BSRS4-XTEN713- | 511 | aCEA_scFv-CCD51-BSRS4-XTEN713- |
| | DM1 | | MMAE |
| 80 | aEpCAM_scFv-CCD51-BSRS4-XTEN713- | 512 | aEpCAM_scFv-CCD51-BSRS4-XTEN713- |
| | DM1 | | MMAE |
| 81 | aHER2_scFv-CCD1-BSRS4-XTEN713- | 513 | aHER2_scFv-CCD1-BSRS4-XTEN713- |
| | DM1 | | MMAE |
| 82 | Folate-CCD1-BSRS4-XTEN713-DM1 | 514 | Folate-CCD1-BSRS4-XTEN713-MMAE |
| 83 | aCEA_scFv-CCD1-BSRS4-XTEN713- | 515 | aCEA_scFv-CCD1-BSRS4-XTEN713-MMAE |
| | DM1 | | |
| 84 | aEpCAM_scFv-CCD1-BSRS4-XTEN713- | 516 | aEpCAM_scFv-CCD1-BSRS4-XTEN713- |
| | DM1 | | MMAE |
| 85 | aHER2_scFv-CCD7-BSRS4-XTEN713- | 517 | aHER2_scFv-CCD7-BSRS4-XTEN713- |
| | DM1 | | MMAE |
| 86 | Folate-CCD7-BSRS4-XTEN713-DM1 | 518 | Folate-CCD7-BSRS4-XTEN713-MMAE |
| 87 | aCEA_scFv-CCD7-BSRS4-XTEN713- | 519 | aCEA_scFv-CCD7-BSRS4-XTEN713-MMAE |
| | DM1 | | |
| 88 | aEpCAM_scFv-CCD7-BSRS4-XTEN713- | 520 | aEpCAM_scFv-CCD7-BSRS4-XTEN713- |
| | DM1 | | MMAE |
| 89 | aHER2_scFv-CCD12-BSRS4-XTEN713- | 521 | aHER2_scFv-CCD12-BSRS4-XTEN713- |
| | DM1 | | MMAE |
| 90 | Folate-CCD12-BSRS4-XTEN713-DM1 | 522 | Folate-CCD12-BSRS4-XTEN713-MMAE |
| 91 | aCEA_scFv-CCD12-BSRS4-XTEN713- | 523 | aCEA_scFv-CCD12-BSRS4-XTEN713- |
| | DM1 | | MMAE |
| 92 | aEpCAM_scFv-CCD12-BSRS4-XTEN713- | 524 | aEpCAM_scFv-CCD12-BSRS4-XTEN713- |
| | DM1 | | MMAE |
| 93 | aHER2_scFv-CCD16-BSRS4-XTEN713- | 525 | aHER2_scFv-CCD16-BSRS4-XTEN713- |
| | DM1 | | MMAE |
| 94 | Folate-CCD16-BSRS4-XTEN713-DM1 | 526 | Folate-CCD16-BSRS4-XTEN713-MMAE |
| 95 | aCEA_scFv-CCD16-BSRS4-XTEN713- | 527 | aCEA_scFv-CCD16-BSRS4-XTEN713- |
| | DM1 | | MMAE |
| 96 | aEpCAM_scFv-CCD16-BSRS4-XTEN713- | 528 | aEpCAM_scFv-CCD16-BSRS4-XTEN713- |
| | DM1 | | MMAE |
| 97 | aHER2_scFv-CCD50-BSRS5-XTEN713- | 529 | aHER2_scFv-CCD50-BSRS5-XTEN713- |
| | DM1 | | MMAE |
| 98 | Folate-CCD50-BSRS5-XTEN713-DM1 | 530 | Folate-CCD50-BSRS5-XTEN713-MMAE |
| 99 | aCEA_scFv-CCD50-BSRS5-XTEN713- | 531 | aCEA_scFv-CCD50-BSRS5-XTEN713- |
| | DM1 | | MMAE |
| 100 | aEpCAM_scFv-CCD50-BSRS5-XTEN713- | 532 | aEpCAM_scFv-CCD50-BSRS5-XTEN713- |
| | DM1 | | MMAE |
| 101 | aHER2_scFv-CCD51-BSRS5-XTEN713- | 533 | aHER2_scFv-CCD51-BSRS5-XTEN713- |
| | DM1 | | MMAE |
| 102 | Folate-CCD51-BSRS5-XTEN713-DM1 | 534 | Folate-CCD51-BSRS5-XTEN713-MMAE |
| 103 | aCEA_scFv-CCD51-BSRS5-XTEN713- | 535 | aCEA_scFv-CCD51-BSRS5-XTEN713- |
| | DM1 | | MMAE |
| 104 | aEpCAM_scFv-CCD51-BSRS5-XTEN713- | 536 | aEpCAM_scFv-CCD51-BSRS5-XTEN713- |
| | DM1 | | MMAE |
| 105 | aHER2_scFv-CCD1-BSRS5-XTEN713- | 537 | aHER2_scFv-CCD1-BSRS5-XTEN713- |
| | DM1 | | MMAE |
| 106 | Folate-CCD1-BSRS5-XTEN713-DM1 | 538 | Folate-CCD1-BSRS5-XTEN713-MMAE |
| 107 | aCEA_scFv-CCD1-BSRS5-XTEN713- | 539 | aCEA_scFv-CCD1-BSRS5-XTEN713-MMAE |
| | DM1 | | |
| 108 | aEpCAM_scFv-CCD1-BSRS5-XTEN713- | 540 | aEpCAM_scFv-CCD1-BSRS5-XTEN713- |
| | DM1 | | MMAE |
| 109 | aHER2_scFv-CCD7-BSRS5-XTEN713- | 541 | aHER2_scFv-CCD7-BSRS5-XTEN713- |
| | DM1 | | MMAE |
| 110 | Folate-CCD7-BSRS5-XTEN713-DM1 | 542 | Folate-CCD7-BSRS5-XTEN713-MMAE |
| 111 | aCEA_scFv-CCD7-BSRS5-XTEN713- | 543 | aCEA_scFv-CCD7-BSRS5-XTEN713-MMAE |
| | DM1 | | |
| 112 | aEpCAM_scFv-CCD7-BSRS5-XTEN713- | 544 | aEpCAM_scFv-CCD7-BSRS5-XTEN713- |
| | DM1 | | MMAE |
| 113 | aHER2_scFv-CCD12-BSRS5-XTEN713- | 545 | aHER2_scFv-CCD12-BSRS5-XTEN713- |
| | DM1 | | MMAE |
| 114 | Folate-CCD12-BSRS5-XTEN713-DM1 | 546 | Folate-CCD12-BSRS5-XTEN713-MMAE |
| 115 | aCEA_scFv-CCD12-BSRS5-XTEN713- | 547 | aCEA_scFv-CCD12-BSRS5-XTEN713- |
| | DM1 | | MMAE |
| 116 | aEpCAM_scFv-CCD12-BSRS5-XTEN713- | 548 | aEpCAM_scFv-CCD12-BSRS5-XTEN713- |
| | DM1 | | MMAE |
| 117 | aHER2_scFv-CCD16-BSRS5-XTEN713- | 549 | aHER2_scFv-CCD16-BSRS5-XTEN713- |
| | DM1 | | MMAE |
| 118 | Folate-CCD16-BSRS5-XTEN713-DM1 | 550 | Folate-CCD16-BSRS5-XTEN713-MMAE |
| 119 | aCEA_scFv-CCD16-BSRS5-XTEN713- | 551 | aCEA_scFv-CCD16-BSRS5-XTEN713- |
| | DM1 | | MMAE |
| 120 | aEpCAM_scFv-CCD16-BSRS5-XTEN713- | 552 | aEpCAM_scFv-CCD16-BSRS5-XTEN713- |
| | DM1 | | MMAE |
| 121 | aHER2_scFv-CCD50-BSRS6-XTEN713- | 553 | aHER2_scFv-CCD50-BSRS6-XTEN713- |
| | DM1 | | MMAE |
| 122 | Folate-CCD50-BSRS6-XTEN713-DM1 | 554 | Folate-CCD50-BSRS6-XTEN713-MMAE |
| 123 | aCEA_scFv-CCD50-BSRS6-XTEN713- | 555 | aCEA_scFv-CCD50-BSRS6-XTEN713- |
| | DM1 | | MMAE |
| 124 | aEpCAM_scFv-CCD50-BSRS6-XTEN713- | 556 | aEpCAM_scFv-CCD50-BSRS6-XTEN713- |
| | DM1 | | MMAE |
| 125 | aHER2_scFv-CCD51-BSRS6-XTEN713- | 557 | aHER2_scFv-CCD51-BSRS6-XTEN713- |
| | DM1 | | MMAE |
| 126 | Folate-CCD51-BSRS6-XTEN713-DM1 | 558 | Folate-CCD51-BSRS6-XTEN713-MMAE |
| 127 | aCEA_scFv-CCD51-BSRS6-XTEN713- | 559 | aCEA_scFv-CCD51-BSRS6-XTEN713- |
| | DM1 | | MMAE |
| 128 | aEpCAM_scFv-CCD51-BSRS6-XTEN713- | 560 | aEpCAM_scFv-CCD51-BSRS6-XTEN713- |
| | DM1 | | MMAE |
| 129 | aHER2_scFv-CCD1-BSRS6-XTEN713- | 561 | aHER2_scFv-CCD1-BSRS6-XTEN713- |
| | DM1 | | MMAE |
| 130 | Folate-CCD1-BSRS6-XTEN713-DM1 | 562 | Folate-CCD1-BSRS6-XTEN713-MMAE |
| 131 | aCEA_scFv-CCD1-BSRS6-XTEN713- | 563 | aCEA_scFv-CCD1-BSRS6-XTEN713-MMAE |
| | DM1 | | |
| 132 | aEpCAM_scFv-CCD1-BSRS6-XTEN713- | 564 | aEpCAM_scFv-CCD1-BSRS6-XTEN713- |
| | DM1 | | MMAE |
| 133 | aHER2_scFv-CCD7-BSRS6-XTEN713- | 565 | aHER2_scFv-CCD7-BSRS6-XTEN713- |
| | DM1 | | MMAE |
| 134 | Folate-CCD7-BSRS6-XTEN713-DM1 | 566 | Folate-CCD7-BSRS6-XTEN713-MMAE |
| 135 | aCEA_scFv-CCD7-BSRS6-XTEN713- | 567 | aCEA_scFv-CCD7-BSRS6-XTEN713-MMAE |
| | DM1 | | |
| 136 | aEpCAM_scFv-CCD7-BSRS6-XTEN713- | 568 | aEpCAM_scFv-CCD7-BSRS6-XTEN713- |
| | DM1 | | MMAE |
| 137 | aHER2_scFv-CCD12-BSRS6-XTEN713- | 569 | aHER2_scFv-CCD12-BSRS6-XTEN713- |
| | DM1 | | MMAE |
| 138 | Folate-CCD12-BSRS6-XTEN713-DM1 | 570 | Folate-CCD12-BSRS6-XTEN713-MMAE |
| 139 | aCEA_scFv-CCD12-BSRS6-XTEN713- | 571 | aCEA_scFv-CCD12-BSRS6-XTEN713- |
| | DM1 | | MMAE |
| 140 | aEpCAM_scFv-CCD12-BSRS6-XTEN713- | 572 | aEpCAM_scFv-CCD12-BSRS6-XTEN713- |
| | DM1 | | MMAE |
| 141 | aHER2_scFv-CCD16-BSRS6-XTEN713- | 573 | aHER2_scFv-CCD16-BSRS6-XTEN713- |
| | DM1 | | MMAE |
| 142 | Folate-CCD16-BSRS6-XTEN713-DM1 | 574 | Folate-CCD16-BSRS6-XTEN713-MMAE |
| 143 | aCEA_scFv-CCD16-BSRS6-XTEN713- | 575 | aCEA_scFv-CCD16-BSRS6-XTEN713- |
| | DM1 | | MMAE |
| 144 | aEpCAM_scFv-CCD16-BSRS6-XTEN713- | 576 | aEpCAM_scFv-CCD16-BSRS6-XTEN713- |
| | DM1 | | MMAE |
| 145 | aHER2_scFv-CCD50-BSRS1-XTEN864- | 577 | aHER2_scFv-CCD50-BSRS1-XTEN864- |
| | DM1 | | MMAE |
| 146 | Folate-CCD50-BSRS1-XTEN864-DM1 | 578 | Folate-CCD50-BSRS1-XTEN864-MMAE |
| 147 | aCEA_scFv-CCD50-BSRS1-XTEN864- | 579 | aCEA_scFv-CCD50-BSRS1-XTEN864- |
| | DM1 | | MMAE |
| 148 | aEpCAM_scFv-CCD50-BSRS1-XTEN864- | 580 | aEpCAM_scFv-CCD50-BSRS1-XTEN864- |
| | DM1 | | MMAE |
| 149 | aHER2_scFv-CCD51-BSRS1-XTEN864- | 581 | aHER2_scFv-CCD51-BSRS1-XTEN864- |
| | DM1 | | MMAE |
| 150 | Folate-CCD51-BSRS1-XTEN864-DM1 | 582 | Folate-CCD51-BSRS1-XTEN864-MMAE |
| 151 | aCEA_scFv-CCD51-BSRS1-XTEN864- | 583 | aCEA_scFv-CCD51-BSRS1-XTEN864- |
| | DM1 | | MMAE |
| 152 | aEpCAM_scFv-CCD51-BSRS1-XTEN864- | 584 | aEpCAM_scFv-CCD51-BSRS1-XTEN864- |
| | DM1 | | MMAE |
| 153 | aHER2_scFv-CCD1-BSRS1-XTEN864- | 585 | aHER2_scFv-CCD1-BSRS1-XTEN864- |
| | DM1 | | MMAE |
| 154 | Folate-CCD1-BSRS1-XTEN864-DM1 | 586 | Folate-CCD1-BSRS1-XTEN864-MMAE |
| 155 | aCEA_scFv-CCD1-BSRS1-XTEN864- | 587 | aCEA_scFv-CCD1-BSRS1-XTEN864-MMAE |
| | DM1 | | |
| 156 | aEpCAM_scFv-CCD1-BSRS1-XTEN864- | 588 | aEpCAM_scFv-CCD1-BSRS1-XTEN864- |
| | DM1 | | MMAE |
| 157 | aHER2_scFv-CCD7-BSRS1-XTEN864- | 589 | aHER2_scFv-CCD7-BSRS1-XTEN864- |
| | DM1 | | MMAE |
| 158 | Folate-CCD7-BSRS1-XTEN864-DM1 | 590 | Folate-CCD7-BSRS1-XTEN864-MMAE |
| 159 | aCEA_scFv-CCD7-BSRS1-XTEN864- | 591 | aCEA_scFv-CCD7-BSRS1-XTEN864-MMAE |
| | DM1 | | |
| 160 | aEpCAM_scFv-CCD7-BSRS1-XTEN864- | 592 | aEpCAM_scFv-CCD7-BSRS1-XTEN864- |
| | DM1 | | MMAE |
| 161 | aHER2_scFv-CCD12-BSRS1-XTEN864- | 593 | aHER2_scFv-CCD12-BSRS1-XTEN864- |
| | DM1 | | MMAE |
| 162 | Folate-CCD12-BSRS1-XTEN864-DM1 | 594 | Folate-CCD12-BSRS1-XTEN864-MMAE |
| 163 | aCEA_scFv-CCD12-BSRS1-XTEN864- | 595 | aCEA_scFv-CCD12-BSRS1-XTEN864- |
| | DM1 | | MMAE |
| 164 | aEpCAM_scFv-CCD12-BSRS1-XTEN864- | 596 | aEpCAM_scFv-CCD12-BSRS1-XTEN864- |
| | DM1 | | MMAE |
| 165 | aHER2_scFv-CCD16-BSRS1-XTEN864- | 597 | aHER2_scFv-CCD16-BSRS1-XTEN864- |
| | DM1 | | MMAE |
| 166 | Folate-CCD16-BSRS1-XTEN864-DM1 | 598 | Folate-CCD16-BSRS1-XTEN864-MMAE |
| 167 | aCEA_scFv-CCD16-BSRS1-XTEN864- | 599 | aCEA_scFv-CCD16-BSRS1-XTEN864- |
| | DM1 | | MMAE |
| 168 | aEpCAM_scFv-CCD16-BSRS1-XTEN864- | 600 | aEpCAM_scFv-CCD16-BSRS1-XTEN864- |
| | DM1 | | MMAE |
| 169 | aHER2_scFv-CCD50-BSRS2-XTEN864- | 601 | aHER2_scFv-CCD50-BSRS2-XTEN864- |
| | DM1 | | MMAE |
| 170 | Folate-CCD50-BSRS2-XTEN864-DM1 | 602 | Folate-CCD50-BSRS2-XTEN864-MMAE |
| 171 | aCEA_scFv-CCD50-BSRS2-XTEN864- | 603 | aCEA_scFv-CCD50-BSRS2-XTEN864- |
| | DM1 | | MMAE |
| 172 | aEpCAM_scFv-CCD50-BSRS2-XTEN864- | 604 | aEpCAM_scFv-CCD50-BSRS2-XTEN864- |
| | DM1 | | MMAE |
| 173 | aHER2_scFv-CCD51-BSRS2-XTEN864- | 605 | aHER2_scFv-CCD51-BSRS2-XTEN864- |
| | DM1 | | MMAE |
| 174 | Folate-CCD51-BSRS2-XTEN864-DM1 | 606 | Folate-CCD51-BSRS2-XTEN864-MMAE |
| 175 | aCEA_scFv-CCD51-BSRS2-XTEN864- | 607 | aCEA_scFv-CCD51-BSRS2-XTEN864- |
| | DM1 | | MMAE |
| 176 | aEpCAM_scFv-CCD51-BSRS2-XTEN864- | 608 | aEpCAM_scFv-CCD51-BSRS2-XTEN864- |
| | DM1 | | MMAE |
| 177 | aHER2_scFv-CCD1-BSRS2-XTEN864- | 609 | aHER2_scFv-CCD1-BSRS2-XTEN864- |
| | DM1 | | MMAE |
| 178 | Folate-CCD1-BSRS2-XTEN864-DM1 | 610 | Folate-CCD1-BSRS2-XTEN864-MMAE |
| 179 | aCEA_scFv-CCD1-BSRS2-XTEN864- | 611 | aCEA_scFv-CCD1-BSRS2-XTEN864-MMAE |
| | DM1 | | |
| 180 | aEpCAM_scFv-CCD1-BSRS2-XTEN864- | 612 | aEpCAM_scFv-CCD1-BSRS2-XTEN864- |
| | DM1 | | MMAE |
| 181 | aHER2_scFv-CCD7-BSRS2-XTEN864- | 613 | aHER2_scFv-CCD7-BSRS2-XTEN864- |
| | DM1 | | MMAE |
| 182 | Folate-CCD7-BSRS2-XTEN864-DM1 | 614 | Folate-CCD7-BSRS2-XTEN864-MMAE |
| 183 | aCEA_scFv-CCD7-BSRS2-XTEN864- | 615 | aCEA_scFv-CCD7-BSRS2-XTEN864-MMAE |
| | DM1 | | |
| 184 | aEpCAM_scFv-CCD7-BSRS2-XTEN864- | 616 | aEpCAM_scFv-CCD7-BSRS2-XTEN864- |
| | DM1 | | MMAE |
| 185 | aHER2_scFv-CCD12-BSRS2-XTEN864- | 617 | aHER2_scFv-CCD12-BSRS2-XTEN864- |
| | DM1 | | MMAE |
| 186 | Folate-CCD12-BSRS2-XTEN864-DM1 | 618 | Folate-CCD12-BSRS2-XTEN864-MMAE |
| 187 | aCEA_scFv-CCD12-BSRS2-XTEN864- | 619 | aCEA_scFv-CCD12-BSRS2-XTEN864- |
| | DM1 | | MMAE |
| 188 | aEpCAM_scFv-CCD12-BSRS2-XTEN864- | 620 | aEpCAM_scFv-CCD12-BSRS2-XTEN864- |
| | DM1 | | MMAE |
| 189 | aHER2_scFv-CCD16-BSRS2-XTEN864- | 621 | aHER2_scFv-CCD16-BSRS2-XTEN864- |
| | DM1 | | MMAE |
| 190 | Folate-CCD16-BSRS2-XTEN864-DM1 | 622 | Folate-CCD16-BSRS2-XTEN864-MMAE |
| 191 | aCEA_scFv-CCD16-BSRS2-XTEN864- | 623 | aCEA_scFv-CCD16-BSRS2-XTEN864- |
| | DM1 | | MMAE |
| 192 | aEpCAM_scFv-CCD16-BSRS2-XTEN864- | 624 | aEpCAM_scFv-CCD16-BSRS2-XTEN864- |
| | DM1 | | MMAE |
| 193 | aHER2_scFv-CCD50-BSRS3-XTEN864- | 625 | aHER2_scFv-CCD50-BSRS3-XTEN864- |
| | DM1 | | MMAE |
| 194 | Folate-CCD50-BSRS3-XTEN864-DM1 | 626 | Folate-CCD50-BSRS3-XTEN864-MMAE |
| 195 | aCEA_scFv-CCD50-BSRS3-XTEN864- | 627 | aCEA_scFv-CCD50-BSRS3-XTEN864- |
| | DM1 | | MMAE |
| 196 | aEpCAM_scFv-CCD50-BSRS3-XTEN864- | 628 | aEpCAM_scFv-CCD50-BSRS3-XTEN864- |
| | DM1 | | MMAE |
| 197 | aHER2_scFv-CCD51-BSRS3-XTEN864- | 629 | aHER2_scFv-CCD51-BSRS3-XTEN864- |
| | DM1 | | MMAE |
| 198 | Folate-CCD51-BSRS3-XTEN864-DM1 | 630 | Folate-CCD51-BSRS3-XTEN864-MMAE |
| 199 | aCEA_scFv-CCD51-BSRS3-XTEN864- | 631 | aCEA_scFv-CCD51-BSRS3-XTEN864- |
| | DM1 | | MMAE |
| 200 | aEpCAM_scFv-CCD51-BSRS3-XTEN864- | 632 | aEpCAM_scFv-CCD51-BSRS3-XTEN864- |
| | DM1 | | MMAE |
| 201 | aHER2_scFv-CCD1-BSRS3-XTEN864- | 633 | aHER2_scFv-CCD1-BSRS3-XTEN864- |
| | DM1 | | MMAE |
| 202 | Folate-CCD1-BSRS3-XTEN864-DM1 | 634 | Folate-CCD1-BSRS3-XTEN864-MMAE |
| 203 | aCEA_scFv-CCD1-BSRS3-XTEN864- | 635 | aCEA_scFv-CCD1-BSRS3-XTEN864-MMAE |
| | DM1 | | |
| 204 | aEpCAM_scFv-CCD1-BSRS3-XTEN864- | 636 | aEpCAM_scFv-CCD1-BSRS3-XTEN864- |
| | DM1 | | MMAE |
| 205 | aHER2_scFv-CCD7-BSRS3-XTEN864- | 637 | aHER2_scFv-CCD7-BSRS3-XTEN864- |
| | DM1 | | MMAE |
| 206 | Folate-CCD7-BSRS3-XTEN864-DM1 | 638 | Folate-CCD7-BSRS3-XTEN864-MMAE |
| 207 | aCEA_scFv-CCD7-BSRS3-XTEN864- | 639 | aCEA_scFv-CCD7-BSRS3-XTEN864-MMAE |
| | DM1 | | |
| 208 | aEpCAM_scFv-CCD7-BSRS3-XTEN864- | 640 | aEpCAM_scFv-CCD7-BSRS3-XTEN864- |
| | DM1 | | MMAE |
| 209 | aHER2_scFv-CCD12-BSRS3-XTEN864- | 641 | aHER2_scFv-CCD12-BSRS3-XTEN864- |
| | DM1 | | MMAE |
| 210 | Folate-CCD12-BSRS3-XTEN864-DM1 | 642 | Folate-CCD12-BSRS3-XTEN864-MMAE |
| 211 | aCEA_scFv-CCD12-BSRS3-XTEN864- | 643 | aCEA_scFv-CCD12-BSRS3-XTEN864- |
| | DM1 | | MMAE |
| 212 | aEpCAM_scFv-CCD12-BSRS3-XTEN864- | 644 | aEpCAM_scFv-CCD12-BSRS3-XTEN864- |
| | DM1 | | MMAE |
| 213 | aHER2_scFv-CCD16-BSRS3-XTEN864- | 645 | aHER2_scFv-CCD16-BSRS3-XTEN864- |
| | DM1 | | MMAE |
| 214 | Folate-CCD16-BSRS3-XTEN864-DM1 | 646 | Folate-CCD16-BSRS3-XTEN864-MMAE |
| 215 | aCEA_scFv-CCD16-BSRS3-XTEN864- | 647 | aCEA_scFv-CCD16-BSRS3-XTEN864- |
| | DM1 | | MMAE |
| 216 | aEpCAM_scFv-CCD16-BSRS3-XTEN864- | 648 | aEpCAM_scFv-CCD16-BSRS3-XTEN864- |
| | DM1 | | MMAE |
| 217 | aHER2_scFv-CCD50-BSRS4-XTEN864- | 649 | aHER2_scFv-CCD50-BSRS4-XTEN864- |
| | DM1 | | MMAE |
| 218 | Folate-CCD50-BSRS4-XTEN864-DM1 | 650 | Folate-CCD50-BSRS4-XTEN864-MMAE |
| 219 | aCEA_scFv-CCD50-BSRS4-XTEN864- | 651 | aCEA_scFv-CCD50-BSRS4-XTEN864- |
| | DM1 | | MMAE |
| 220 | aEpCAM_scFv-CCD50-BSRS4-XTEN864- | 652 | aEpCAM_scFv-CCD50-BSRS4-XTEN864- |
| | DM1 | | MMAE |
| 221 | aHER2_scFv-CCD51-BSRS4-XTEN864- | 653 | aHER2_scFv-CCD51-BSRS4-XTEN864- |
| | DM1 | | MMAE |
| 222 | Folate-CCD51-BSRS4-XTEN864-DM1 | 654 | Folate-CCD51-BSRS4-XTEN864-MMAE |
| 223 | aCEA_scFv-CCD51-BSRS4-XTEN864- | 655 | aCEA_scFv-CCD51-BSRS4-XTEN864- |
| | DM1 | | MMAE |
| 224 | aEpCAM_scFv-CCD51-BSRS4-XTEN864- | 656 | aEpCAM_scFv-CCD51-BSRS4-XTEN864- |
| | DM1 | | MMAE |
| 225 | aHER2_scFv-CCD1-BSRS4-XTEN864- | 657 | aHER2_scFv-CCD1-BSRS4-XTEN864- |
| | DM1 | | MMAE |
| 226 | Folate-CCD1-BSRS4-XTEN864-DM1 | 658 | Folate-CCD1-BSRS4-XTEN864-MMAE |
| 227 | aCEA_scFv-CCD1-BSRS4-XTEN864- | 659 | aCEA_scFv-CCD1-BSRS4-XTEN864-MMAE |
| | DM1 | | |
| 228 | aEpCAM_scFv-CCD1-BSRS4-XTEN864- | 660 | aEpCAM_scFv-CCD1-BSRS4-XTEN864- |
| | DM1 | | MMAE |
| 229 | aHER2_scFv-CCD7-BSRS4-XTEN864- | 661 | aHER2_scFv-CCD7-BSRS4-XTEN864- |
| | DM1 | | MMAE |
| 230 | Folate-CCD7-BSRS4-XTEN864-DM1 | 662 | Folate-CCD7-BSRS4-XTEN864-MMAE |
| 231 | aCEA_scFv-CCD7-BSRS4-XTEN864- | 663 | aCEA_scFv-CCD7-BSRS4-XTEN864-MMAE |
| | DM1 | | |
| 232 | aEpCAM_scFv-CCD7-BSRS4-XTEN864- | 664 | aEpCAM_scFv-CCD7-BSRS4-XTEN864- |
| | DM1 | | MMAE |
| 233 | aHER2_scFv-CCD12-BSRS4-XTEN864- | 665 | aHER2_scFv-CCD12-BSRS4-XTEN864- |
| | DM1 | | MMAE |
| 234 | Folate-CCD12-BSRS4-XTEN864-DM1 | 666 | Folate-CCD12-BSRS4-XTEN864-MMAE |
| 235 | aCEA_scFv-CCD12-BSRS4-XTEN864- | 667 | aCEA_scFv-CCD12-BSRS4-XTEN864- |
| | DM1 | | MMAE |
| 236 | aEpCAM_scFv-CCD12-BSRS4-XTEN864- | 668 | aEpCAM_scFv-CCD12-BSRS4-XTEN864- |
| | DM1 | | MMAE |
| 237 | aHER2_scFv-CCD16-BSRS4-XTEN864- | 669 | aHER2_scFv-CCD16-BSRS4-XTEN864- |
| | DM1 | | MMAE |
| 238 | Folate-CCD16-BSRS4-XTEN864-DM1 | 670 | Folate-CCD16-BSRS4-XTEN864-MMAE |
| 239 | aCEA_scFv-CCD16-BSRS4-XTEN864- | 671 | aCEA_scFv-CCD16-BSRS4-XTEN864- |
| | DM1 | | MMAE |
| 240 | aEpCAM_scFv-CCD16-BSRS4-XTEN864- | 672 | aEpCAM_scFv-CCD16-BSRS4-XTEN864- |
| | DM1 | | MMAE |
| 241 | aHER2_scFv-CCD50-BSRS5-XTEN864- | 673 | aHER2_scFv-CCD50-BSRS5-XTEN864- |
| | DM1 | | MMAE |
| 242 | Folate-CCD50-BSRS5-XTEN864-DM1 | 674 | Folate-CCD50-BSRS5-XTEN864-MMAE |
| 243 | aCEA_scFv-CCD50-BSRS5-XTEN864- | 675 | aCEA_scFv-CCD50-BSRS5-XTEN864- |
| | DM1 | | MMAE |
| 244 | aEpCAM_scFv-CCD50-BSRS5-XTEN864- | 676 | aEpCAM_scFv-CCD50-BSRS5-XTEN864- |
| | DM1 | | MMAE |
| 245 | aHER2_scFv-CCD51-BSRS5-XTEN864- | 677 | aHER2_scFv-CCD51-BSRS5-XTEN864- |
| | DM1 | | MMAE |
| 246 | Folate-CCD51-BSRS5-XTEN864-DM1 | 678 | Folate-CCD51-BSRS5-XTEN864-MMAE |
| 247 | aCEA_scFv-CCD51-BSRS5-XTEN864- | 679 | aCEA_scFv-CCD51-BSRS5-XTEN864- |
| | DM1 | | MMAE |
| 248 | aEpCAM_scFv-CCD51-BSRS5-XTEN864- | 680 | aEpCAM_scFv-CCD51-BSRS5-XTEN864- |
| | DM1 | | MMAE |
| 249 | aHER2_scFv-CCD1-BSRS5-XTEN864- | 681 | aHER2_scFv-CCD1-BSRS5-XTEN864- |
| | DM1 | | MMAE |
| 250 | Folate-CCD1-BSRS5-XTEN864-DM1 | 682 | Folate-CCD1-BSRS5-XTEN864-MMAE |
| 251 | aCEA_scFv-CCD1-BSRS5-XTEN864- | 683 | aCEA_scFv-CCD1-BSRS5-XTEN864-MMAE |
| | DM1 | | |
| 252 | aEpCAM_scFv-CCD1-BSRS5-XTEN864- | 684 | aEpCAM_scFv-CCD1-BSRS5-XTEN864- |
| | DM1 | | MMAE |
| 253 | aHER2_scFv-CCD7-BSRS5-XTEN864- | 685 | aHER2_scFv-CCD7-BSRS5-XTEN864- |
| | DM1 | | MMAE |
| 254 | Folate-CCD7-BSRS5-XTEN864-DM1 | 686 | Folate-CCD7-BSRS5-XTEN864-MMAE |
| 255 | aCEA_scFv-CCD7-BSRS5-XTEN864- | 687 | aCEA_scFv-CCD7-BSRS5-XTEN864-MMAE |
| | DM1 | | |
| 256 | aEpCAM_scFv-CCD7-BSRS5-XTEN864- | 688 | aEpCAM_scFv-CCD7-BSRS5-XTEN864- |
| | DM1 | | MMAE |
| 257 | aHER2_scFv-CCD12-BSRS5-XTEN864- | 689 | aHER2_scFv-CCD12-BSRS5-XTEN864- |
| | DM1 | | MMAE |
| 258 | Folate-CCD12-BSRS5-XTEN864-DM1 | 690 | Folate-CCD12-BSRS5-XTEN864-MMAE |
| 259 | aCEA_scFv-CCD12-BSRS5-XTEN864- | 691 | aCEA_scFv-CCD12-BSRS5-XTEN864- |
| | DM1 | | MMAE |
| 260 | aEpCAM_scFv-CCD12-BSRS5-XTEN864- | 692 | aEpCAM_scFv-CCD12-BSRS5-XTEN864- |
| | DM1 | | MMAE |
| 261 | aHER2_scFv-CCD16-BSRS5-XTEN864- | 693 | aHER2_scFv-CCD16-BSRS5-XTEN864- |
| | DM1 | | MMAE |
| 262 | Folate-CCD16-BSRS5-XTEN864-DM1 | 694 | Folate-CCD16-BSRS5-XTEN864-MMAE |
| 263 | aCEA_scFv-CCD16-BSRS5-XTEN864- | 695 | aCEA_scFv-CCD16-BSRS5-XTEN864- |
| | DM1 | | MMAE |
| 264 | aEpCAM_scFv-CCD16-BSRS5-XTEN864- | 696 | aEpCAM_scFv-CCD16-BSRS5-XTEN864- |
| | DM1 | | MMAE |
| 265 | aHER2_scFv-CCD50-BSRS6-XTEN864- | 697 | aHER2_scFv-CCD50-BSRS6-XTEN864- |
| | DM1 | | MMAE |
| 266 | Folate-CCD50-BSRS6-XTEN864-DM1 | 698 | Folate-CCD50-BSRS6-XTEN864-MMAE |
| 267 | aCEA_scFv-CCD50-BSRS6-XTEN864- | 699 | aCEA_scFv-CCD50-BSRS6-XTEN864- |
| | DM1 | | MMAE |
| 268 | aEpCAM_scFv-CCD50-BSRS6-XTEN864- | 700 | aEpCAM_scFv-CCD50-BSRS6-XTEN864- |
| | DM1 | | MMAE |
| 269 | aHER2_scFv-CCD51-BSRS6-XTEN864- | 701 | aHER2_scFv-CCD51-BSRS6-XTEN864- |
| | DM1 | | MMAE |
| 270 | Folate-CCD51-BSRS6-XTEN864-DM1 | 702 | Folate-CCD51-BSRS6-XTEN864-MMAE |
| 271 | aCEA_scFv-CCD51-BSRS6-XTEN864- | 703 | aCEA_scFv-CCD51-BSRS6-XTEN864- |
| | DM1 | | MMAE |
| 272 | aEpCAM_scFv-CCD51-BSRS6-XTEN864- | 704 | aEpCAM_scFv-CCD51-BSRS6-XTEN864- |
| | DM1 | | MMAE |
| 273 | aHER2_scFv-CCD1-BSRS6-XTEN864- | 705 | aHER2_scFv-CCD1-BSRS6-XTEN864- |
| | DM1 | | MMAE |
| 274 | Folate-CCD1-BSRS6-XTEN864-DM1 | 706 | Folate-CCD1-BSRS6-XTEN864-MMAE |
| 275 | aCEA_scFv-CCD1-BSRS6-XTEN864- | 707 | aCEA_scFv-CCD1-BSRS6-XTEN864-MMAE |
| | DM1 | | |
| 276 | aEpCAM_scFv-CCD1-BSRS6-XTEN864- | 708 | aEpCAM_scFv-CCD1-BSRS6-XTEN864- |
| | DM1 | | MMAE |
| 277 | aHER2_scFv-CCD7-BSRS6-XTEN864- | 709 | aHER2_scFv-CCD7-BSRS6-XTEN864- |
| | DM1 | | MMAE |
| 278 | Folate-CCD7-BSRS6-XTEN864-DM1 | 710 | Folate-CCD7-BSRS6-XTEN864-MMAE |
| 279 | aCEA_scFv-CCD7-BSRS6-XTEN864- | 711 | aCEA_scFv-CCD7-BSRS6-XTEN864-MMAE |
| | DM1 | | |
| 280 | aEpCAM_scFv-CCD7-BSRS6-XTEN864- | 712 | aEpCAM_scFv-CCD7-BSRS6-XTEN864- |
| | DM1 | | MMAE |
| 281 | aHER2_scFv-CCD12-BSRS6-XTEN864- | 713 | aHER2_scFv-CCD12-BSRS6-XTEN864- |
| | DM1 | | MMAE |
| 282 | Folate-CCD12-BSRS6-XTEN864-DM1 | 714 | Folate-CCD12-BSRS6-XTEN864-MMAE |
| 283 | aCEA_scFv-CCD12-BSRS6-XTEN864- | 715 | aCEA_scFv-CCD12-BSRS6-XTEN864- |
| | DM1 | | MMAE |
| 284 | aEpCAM_scFv-CCD12-BSRS6-XTEN864- | 716 | aEpCAM_scFv-CCD12-BSRS6-XTEN864- |
| | DM1 | | MMAE |
| 285 | aHER2_scFv-CCD16-BSRS6-XTEN864- | 717 | aHER2_scFv-CCD16-BSRS6-XTEN864- |
| | DM1 | | MMAE |
| 286 | Folate-CCD16-BSRS6-XTEN864-DM1 | 718 | Folate-CCD16-BSRS6-XTEN864-MMAE |
| 287 | aCEA_scFv-CCD16-BSRS6-XTEN864- | 719 | aCEA_scFv-CCD16-BSRS6-XTEN864- |
| | DM1 | | MMAE |
| 288 | aEpCAM_scFv-CCD16-BSRS6-XTEN864- | 720 | aEpCAM_scFv-CCD16-BSRS6-XTEN864- |
| | DM1 | | MMAE |
| 289 | aHER2_scFv-CCD50-BSRS1-XTEN576- | 721 | aHER2_scFv-CCD50-BSRS1-XTEN576- |
| | DM1 | | MMAE |
| 290 | Folate-CCD50-BSRS1-XTEN576-DM1 | 722 | Folate-CCD50-BSRS1-XTEN576-MMAE |
| 291 | aCEA_scFv-CCD50-BSRS1-XTEN576- | 723 | aCEA_scFv-CCD50-BSRS1-XTEN576- |
| | DM1 | | MMAE |
| 292 | aEpCAM_scFv-CCD50-BSRS1-XTEN576- | 724 | aEpCAM_scFv-CCD50-BSRS1-XTEN576- |
| | DM1 | | MMAE |
| 293 | aHER2_scFv-CCD51-BSRS1-XTEN576- | 725 | aHER2_scFv-CCD51-BSRS1-XTEN576- |
| | DM1 | | MMAE |
| 294 | Folate-CCD51-BSRS1-XTEN576-DM1 | 726 | Folate-CCD51-BSRS1-XTEN576-MMAE |
| 295 | aCEA_scFv-CCD51-BSRS1-XTEN576- | 727 | aCEA_scFv-CCD51-BSRS1-XTEN576- |
| | DM1 | | MMAE |
| 296 | aEpCAM_scFv-CCD51-BSRS1-XTEN576- | 728 | aEpCAM_scFv-CCD51-BSRS1-XTEN576- |
| | DM1 | | MMAE |
| 297 | aHER2_scFv-CCD1-BSRS1-XTEN576- | 729 | aHER2_scFv-CCD1-BSRS1-XTEN576- |
| | DM1 | | MMAE |
| 298 | Folate-CCD1-BSRS1-XTEN576-DM1 | 730 | Folate-CCD1-BSRS1-XTEN576-MMAE |
| 299 | aCEA_scFv-CCD1-BSRS1-XTEN576- | 731 | aCEA_scFv-CCD1-BSRS1-XTEN576-MMAE |
| | DM1 | | |
| 300 | aEpCAM_scFv-CCD1-BSRS1-XTEN576- | 732 | aEpCAM_scFv-CCD1-BSRS1-XTEN576- |
| | DM1 | | MMAE |
| 301 | aHER2_scFv-CCD7-BSRS1-XTEN576- | 733 | aHER2_scFv-CCD7-BSRS1-XTEN576- |
| | DM1 | | MMAE |
| 302 | Folate-CCD7-BSRS1-XTEN576-DM1 | 734 | Folate-CCD7-BSRS1-XTEN576-MMAE |
| 303 | aCEA_scFv-CCD7-BSRS1-XTEN576- | 735 | aCEA_scFv-CCD7-BSRS1-XTEN576-MMAE |
| | DM1 | | |
| 304 | aEpCAM_scFv-CCD7-BSRS1-XTEN576- | 736 | aEpCAM_scFv-CCD7-BSRS1-XTEN576- |
| | DM1 | | MMAE |
| 305 | aHER2_scFv-CCD12-BSRS1-XTEN576- | 737 | aHER2_scFv-CCD12-BSRS1-XTEN576- |
| | DM1 | | MMAE |
| 306 | Folate-CCD12-BSRS1-XTEN576-DM1 | 738 | Folate-CCD12-BSRS1-XTEN576-MMAE |
| 307 | aCEA_scFv-CCD12-BSRS1-XTEN576- | 739 | aCEA_scFv-CCD12-BSRS1-XTEN576- |
| | DM1 | | MMAE |
| 308 | aEpCAM_scFv-CCD12-BSRS1-XTEN576- | 740 | aEpCAM_scFv-CCD12-BSRS1-XTEN576- |
| | DM1 | | MMAE |
| 309 | aHER2_scFv-CCD16-BSRS1-XTEN576- | 741 | aHER2_scFv-CCD16-BSRS1-XTEN576- |
| | DM1 | | MMAE |
| 310 | Folate-CCD16-BSRS1-XTEN576-DM1 | 742 | Folate-CCD16-BSRS1-XTEN576-MMAE |
| 311 | aCEA_scFv-CCD16-BSRS1-XTEN576- | 743 | aCEA_scFv-CCD16-BSRS1-XTEN576- |
| | DM1 | | MMAE |
| 312 | aEpCAM_scFv-CCD16-BSRS1-XTEN576- | 744 | aEpCAM_scFv-CCD16-BSRS1-XTEN576- |
| | DM1 | | MMAE |
| 313 | aHER2_scFv-CCD50-BSRS2-XTEN576- | 745 | aHER2_scFv-CCD50-BSRS2-XTEN576- |
| | DM1 | | MMAE |
| 314 | Folate-CCD50-BSRS2-XTEN576-DM1 | 746 | Folate-CCD50-BSRS2-XTEN576-MMAE |
| 315 | aCEA_scFv-CCD50-BSRS2-XTEN576- | 747 | aCEA_scFv-CCD50-BSRS2-XTEN576- |
| | DM1 | | MMAE |
| 316 | aEpCAM_scFv-CCD50-BSRS2-XTEN576- | 748 | aEpCAM_scFv-CCD50-BSRS2-XTEN576- |
| | DM1 | | MMAE |
| 317 | aHER2_scFv-CCD51-BSRS2-XTEN576- | 749 | aHER2_scFv-CCD51-BSRS2-XTEN576- |
| | DM1 | | MMAE |
| 318 | Folate-CCD51-BSRS2-XTEN576-DM1 | 750 | Folate-CCD51-BSRS2-XTEN576-MMAE |
| 319 | aCEA_scFv-CCD51-BSRS2-XTEN576- | 751 | aCEA_scFv-CCD51-BSRS2-XTEN576- |
| | DM1 | | MMAE |
| 320 | aEpCAM_scFv-CCD51-BSRS2-XTEN576- | 752 | aEpCAM_scFv-CCD51-BSRS2-XTEN576- |
| | DM1 | | MMAE |
| 321 | aHER2_scFv-CCD1-BSRS2-XTEN576- | 753 | aHER2_scFv-CCD1-BSRS2-XTEN576- |
| | DM1 | | MMAE |
| 322 | Folate-CCD1-BSRS2-XTEN576-DM1 | 754 | Folate-CCD1-BSRS2-XTEN576-MMAE |
| 323 | aCEA_scFv-CCD1-BSRS2-XTEN576- | 755 | aCEA_scFv-CCD1-BSRS2-XTEN576-MMAE |
| | DM1 | | |
| 324 | aEpCAM_scFv-CCD1-BSRS2-XTEN576- | 756 | aEpCAM_scFv-CCD1-BSRS2-XTEN576- |
| | DM1 | | MMAE |
| 325 | aHER2_scFv-CCD7-BSRS2-XTEN576- | 757 | aHER2_scFv-CCD7-BSRS2-XTEN576- |
| | DM1 | | MMAE |
| 326 | Folate-CCD7-BSRS2-XTEN576-DM1 | 758 | Folate-CCD7-BSRS2-XTEN576-MMAE |
| 327 | aCEA_scFv-CCD7-BSRS2-XTEN576- | 759 | aCEA_scFv-CCD7-BSRS2-XTEN576-MMAE |
| | DM1 | | |
| 328 | aEpCAM_scFv-CCD7-BSRS2-XTEN576- | 760 | aEpCAM_scFv-CCD7-BSRS2-XTEN576- |
| | DM1 | | MMAE |
| 329 | aHER2_scFv-CCD12-BSRS2-XTEN576- | 761 | aHER2_scFv-CCD12-BSRS2-XTEN576- |
| | DM1 | | MMAE |
| 330 | Folate-CCD12-BSRS2-XTEN576-DM1 | 762 | Folate-CCD12-BSRS2-XTEN576-MMAE |
| 331 | aCEA_scFv-CCD12-BSRS2-XTEN576- | 763 | aCEA_scFv-CCD12-BSRS2-XTEN576- |
| | DM1 | | MMAE |
| 332 | aEpCAM_scFv-CCD12-BSRS2-XTEN576- | 764 | aEpCAM_scFv-CCD12-BSRS2-XTEN576- |
| | DM1 | | MMAE |
| 333 | aHER2_scFv-CCD16-BSRS2-XTEN576- | 765 | aHER2_scFv-CCD16-BSRS2-XTEN576- |
| | DM1 | | MMAE |
| 334 | Folate-CCD16-BSRS2-XTEN576-DM1 | 766 | Folate-CCD16-BSRS2-XTEN576-MMAE |
| 335 | aCEA_scFv-CCD16-BSRS2-XTEN576- | 767 | aCEA_scFv-CCD16-BSRS2-XTEN576- |
| | DM1 | | MMAE |
| 336 | aEpCAM_scFv-CCD16-BSRS2-XTEN576- | 768 | aEpCAM_scFv-CCD16-BSRS2-XTEN576- |
| | DM1 | | MMAE |
| 337 | aHER2_scFv-CCD50-BSRS3-XTEN576- | 769 | aHER2_scFv-CCD50-BSRS3-XTEN576- |
| | DM1 | | MMAE |
| 338 | Folate-CCD50-BSRS3-XTEN576-DM1 | 770 | Folate-CCD50-BSRS3-XTEN576-MMAE |
| 339 | aCEA_scFv-CCD50-BSRS3-XTEN576- | 771 | aCEA_scFv-CCD50-BSRS3-XTEN576- |
| | DM1 | | MMAE |
| 340 | aEpCAM_scFv-CCD50-BSRS3-XTEN576- | 772 | aEpCAM_scFv-CCD50-BSRS3-XTEN576- |
| | DM1 | | MMAE |
| 341 | aHER2_scFv-CCD51-BSRS3-XTEN576- | 773 | aHER2_scFv-CCD51-BSRS3-XTEN576- |
| | DM1 | | MMAE |
| 342 | Folate-CCD51-BSRS3-XTEN576-DM1 | 774 | Folate-CCD51-BSRS3-XTEN576-MMAE |
| 343 | aCEA_scFv-CCD51-BSRS3-XTEN576- | 775 | aCEA_scFv-CCD51-BSRS3-XTEN576- |
| | DM1 | | MMAE |
| 344 | aEpCAM_scFv-CCD51-BSRS3-XTEN576- | 776 | aEpCAM_scFv-CCD51-BSRS3-XTEN576- |
| | DM1 | | MMAE |
| 345 | aHER2_scFv-CCD1-BSRS3-XTEN576- | 777 | aHER2_scFv-CCD1-BSRS3-XTEN576- |
| | DM1 | | MMAE |
| 346 | Folate-CCD1-BSRS3-XTEN576-DM1 | 778 | Folate-CCD1-BSRS3-XTEN576-MMAE |
| 347 | aCEA_scFv-CCD1-BSRS3-XTEN576- | 779 | aCEA_scFv-CCD1-BSRS3-XTEN576-MMAE |
| | DM1 | | |
| 348 | aEpCAM_scFv-CCD1-BSRS3-XTEN576- | 780 | aEpCAM_scFv-CCD1-BSRS3-XTEN576- |
| | DM1 | | MMAE |
| 349 | aHER2_scFv-CCD7-BSRS3-XTEN576- | 781 | aHER2_scFv-CCD7-BSRS3-XTEN576- |
| | DM1 | | MMAE |
| 350 | Folate-CCD7-BSRS3-XTEN576-DM1 | 782 | Folate-CCD7-BSRS3-XTEN576-MMAE |
| 351 | aCEA_scFv-CCD7-BSRS3-XTEN576- | 783 | aCEA_scFv-CCD7-BSRS3-XTEN576-MMAE |
| | DM1 | | |
| 352 | aEpCAM_scFv-CCD7-BSRS3-XTEN576- | 784 | aEpCAM_scFv-CCD7-BSRS3-XTEN576- |
| | DM1 | | MMAE |
| 353 | aHER2_scFv-CCD12-BSRS3-XTEN576- | 785 | aHER2_scFv-CCD12-BSRS3-XTEN576- |
| | DM1 | | MMAE |
| 354 | Folate-CCD12-BSRS3-XTEN576-DM1 | 786 | Folate-CCD12-BSRS3-XTEN576-MMAE |
| 355 | aCEA_scFv-CCD12-BSRS3-XTEN576- | 787 | aCEA_scFv-CCD12-BSRS3-XTEN576- |
| | DM1 | | MMAE |
| 356 | aEpCAM_scFv-CCD12-BSRS3-XTEN576- | 788 | aEpCAM_scFv-CCD12-BSRS3-XTEN576- |
| | DM1 | | MMAE |
| 357 | aHER2_scFv-CCD16-BSRS3-XTEN576- | 789 | aHER2_scFv-CCD16-BSRS3-XTEN576- |
| | DM1 | | MMAE |
| 358 | Folate-CCD16-BSRS3-XTEN576-DM1 | 790 | Folate-CCD16-BSRS3-XTEN576-MMAE |
| 359 | aCEA_scFv-CCD16-BSRS3-XTEN576- | 791 | aCEA_scFv-CCD16-BSRS3-XTEN576- |
| | DM1 | | MMAE |
| 360 | aEpCAM_scFv-CCD16-BSRS3-XTEN576- | 792 | aEpCAM_scFv-CCD16-BSRS3-XTEN576- |
| | DM1 | | MMAE |
| 361 | aHER2_scFv-CCD50-BSRS4-XTEN576- | 793 | aHER2_scFv-CCD50-BSRS4-XTEN576- |
| | DM1 | | MMAE |
| 362 | Folate-CCD50-BSRS4-XTEN576-DM1 | 794 | Folate-CCD50-BSRS4-XTEN576-MMAE |
| 363 | aCEA_scFv-CCD50-BSRS4-XTEN576- | 795 | aCEA_scFv-CCD50-BSRS4-XTEN576- |
| | DM1 | | MMAE |
| 364 | aEpCAM_scFv-CCD50-BSRS4-XTEN576- | 796 | aEpCAM_scFv-CCD50-BSRS4-XTEN576- |
| | DM1 | | MMAE |
| 365 | aHER2_scFv-CCD51-BSRS4-XTEN576- | 797 | aHER2_scFv-CCD51-BSRS4-XTEN576- |
| | DM1 | | MMAE |
| 366 | Folate-CCD51-BSRS4-XTEN576-DM1 | 798 | Folate-CCD51-BSRS4-XTEN576-MMAE |
| 367 | aCEA_scFv-CCD51-BSRS4-XTEN576- | 799 | aCEA_scFv-CCD51-BSRS4-XTEN576- |
| | DM1 | | MMAE |
| 368 | aEpCAM_scFv-CCD51-BSRS4-XTEN576- | 800 | aEpCAM_scFv-CCD51-BSRS4-XTEN576- |
| | DM1 | | MMAE |
| 369 | aHER2_scFv-CCD1-BSRS4-XTEN576- | 801 | aHER2_scFv-CCD1-BSRS4-XTEN576- |
| | DM1 | | MMAE |
| 370 | Folate-CCD1-BSRS4-XTEN576-DM1 | 802 | Folate-CCD1-BSRS4-XTEN576-MMAE |
| 371 | aCEA_scFv-CCD1-BSRS4-XTEN576- | 803 | aCEA_scFv-CCD1-BSRS4-XTEN576-MMAE |
| | DM1 | | |
| 372 | aEpCAM_scFv-CCD1-BSRS4-XTEN576- | 804 | aEpCAM_scFv-CCD1-BSRS4-XTEN576- |
| | DM1 | | MMAE |
| 373 | aHER2_scFv-CCD7-BSRS4-XTEN576- | 805 | aHER2_scFv-CCD7-BSRS4-XTEN576- |
| | DM1 | | MMAE |
| 374 | Folate-CCD7-BSRS4-XTEN576-DM1 | 806 | Folate-CCD7-BSRS4-XTEN576-MMAE |
| 375 | aCEA_scFv-CCD7-BSRS4-XTEN576- | 807 | aCEA_scFv-CCD7-BSRS4-XTEN576-MMAE |
| | DM1 | | |
| 376 | aEpCAM_scFv-CCD7-BSRS4-XTEN576- | 808 | aEpCAM_scFv-CCD7-BSRS4-XTEN576- |
| | DM1 | | MMAE |
| 377 | aHER2_scFv-CCD12-BSRS4-XTEN576- | 809 | aHER2_scFv-CCD12-BSRS4-XTEN576- |
| | DM1 | | MMAE |
| 378 | Folate-CCD12-BSRS4-XTEN576-DM1 | 810 | Folate-CCD12-BSRS4-XTEN576-MMAE |
| 379 | aCEA_scFv-CCD12-BSRS4-XTEN576- | 811 | aCEA_scFv-CCD12-BSRS4-XTEN576- |
| | DM1 | | MMAE |
| 380 | aEpCAM_scFv-CCD12-BSRS4-XTEN576- | 812 | aEpCAM_scFv-CCD12-BSRS4-XTEN576- |
| | DM1 | | MMAE |
| 381 | aHER2_scFv-CCD16-BSRS4-XTEN576- | 813 | aHER2_scFv-CCD16-BSRS4-XTEN576- |
| | DM1 | | MMAE |
| 382 | Folate-CCD16-BSRS4-XTEN576-DM1 | 814 | Folate-CCD16-BSRS4-XTEN576-MMAE |
| 383 | aCEA_scFv-CCD16-BSRS4-XTEN576- | 815 | aCEA_scFv-CCD16-BSRS4-XTEN576- |
| | DM1 | | MMAE |
| 384 | aEpCAM_scFv-CCD16-BSRS4-XTEN576- | 816 | aEpCAM_scFv-CCD16-BSRS4-XTEN576- |
| | DM1 | | MMAE |
| 385 | aHER2_scFv-CCD50-BSRS5-XTEN576- | 817 | aHER2_scFv-CCD50-BSRS5-XTEN576- |
| | DM1 | | MMAE |
| 386 | Folate-CCD50-BSRS5-XTEN576-DM1 | 818 | Folate-CCD50-BSRS5-XTEN576-MMAE |
| 387 | aCEA_scFv-CCD50-BSRS5-XTEN576- | 819 | aCEA_scFv-CCD50-BSRS5-XTEN576- |
| | DM1 | | MMAE |
| 388 | aEpCAM_scFv-CCD50-BSRS5-XTEN576- | 820 | aEpCAM_scFv-CCD50-BSRS5-XTEN576- |
| | DM1 | | MMAE |
| 389 | aHER2_scFv-CCD51-BSRS5-XTEN576- | 821 | aHER2_scFv-CCD51-BSRS5-XTEN576- |
| | DM1 | | MMAE |
| 390 | Folate-CCD51-BSRS5-XTEN576-DM1 | 822 | Folate-CCD51-BSRS5-XTEN576-MMAE |
| 391 | aCEA_scFv-CCD51-BSRS5-XTEN576- | 823 | aCEA_scFv-CCD51-BSRS5-XTEN576- |
| | DM1 | | MMAE |
| 392 | aEpCAM_scFv-CCD51-BSRS5-XTEN576- | 824 | aEpCAM_scFv-CCD51-BSRS5-XTEN576- |
| | DM1 | | MMAE |
| 393 | aHER2_scFv-CCD1-BSRS5-XTEN576- | 825 | aHER2_scFv-CCD1-BSRS5-XTEN576- |
| | DM1 | | MMAE |
| 394 | Folate-CCD1-BSRS5-XTEN576-DM1 | 826 | Folate-CCD1-BSRS5-XTEN576-MMAE |
| 395 | aCEA_scFv-CCD1-BSRS5-XTEN576- | 827 | aCEA_scFv-CCD1-BSRS5-XTEN576-MMAE |
| | DM1 | | |
| 396 | aEpCAM_scFv-CCD1-BSRS5-XTEN576- | 828 | aEpCAM_scFv-CCD1-BSRS5-XTEN576- |
| | DM1 | | MMAE |
| 397 | aHER2_scFv-CCD7-BSRS5-XTEN576- | 829 | aHER2_scFv-CCD7-BSRS5-XTEN576- |
| | DM1 | | MMAE |
| 398 | Folate-CCD7-BSRS5-XTEN576-DM1 | 830 | Folate-CCD7-BSRS5-XTEN576-MMAE |
| 399 | aCEA_scFv-CCD7-BSRS5-XTEN576- | 831 | aCEA_scFv-CCD7-BSRS5-XTEN576-MMAE |
| | DM1 | | |
| 400 | aEpCAM_scFv-CCD7-BSRS5-XTEN576- | 832 | aEpCAM_scFv-CCD7-BSRS5-XTEN576- |
| | DM1 | | MMAE |
| 401 | aHER2_scFv-CCD12-BSRS5-XTEN576- | 833 | aHER2_scFv-CCD12-BSRS5-XTEN576- |
| | DM1 | | MMAE |
| 402 | Folate-CCD12-BSRS5-XTEN576-DM1 | 834 | Folate-CCD12-BSRS5-XTEN576-MMAE |
| 403 | aCEA_scFv-CCD12-BSRS5-XTEN576- | 835 | aCEA_scFv-CCD12-BSRS5-XTEN576- |
| | DM1 | | MMAE |
| 404 | aEpCAM_scFv-CCD12-BSRS5-XTEN576- | 836 | aEpCAM_scFv-CCD12-BSRS5-XTEN576- |
| | DM1 | | MMAE |
| 405 | aHER2_scFv-CCD16-BSRS5-XTEN576- | 837 | aHER2_scFv-CCD16-BSRS5-XTEN576- |
| | DM1 | | MMAE |
| 406 | Folate-CCD16-BSRS5-XTEN576-DM1 | 838 | Folate-CCD16-BSRS5-XTEN576-MMAE |
| 407 | aCEA_scFv-CCD16-BSRS5-XTEN576- | 839 | aCEA_scFv-CCD16-BSRS5-XTEN576- |
| | DM1 | | MMAE |
| 408 | aEpCAM_scFv-CCD16-BSRS5-XTEN576- | 840 | aEpCAM_scFv-CCD16-BSRS5-XTEN576- |
| | DM1 | | MMAE |
| 409 | aHER2_scFv-CCD50-BSRS6-XTEN576- | 841 | aHER2_scFv-CCD50-BSRS6-XTEN576- |
| | DM1 | | MMAE |
| 410 | Folate-CCD50-BSRS6-XTEN576-DM1 | 842 | Folate-CCD50-BSRS6-XTEN576-MMAE |
| 411 | aCEA_scFv-CCD50-BSRS6-XTEN576- | 843 | aCEA_scFv-CCD50-BSRS6-XTEN576- |
| | DM1 | | MMAE |
| 412 | aEpCAM_scFv-CCD50-BSRS6-XTEN576- | 844 | aEpCAM_scFv-CCD50-BSRS6-XTEN576- |
| | DM1 | | MMAE |
| 413 | aHER2_scFv-CCD51-BSRS6-XTEN576- | 845 | aHER2_scFv-CCD51-BSRS6-XTEN576- |
| | DM1 | | MMAE |
| 414 | Folate-CCD51-BSRS6-XTEN576-DM1 | 846 | Folate-CCD51-BSRS6-XTEN576-MMAE |
| 415 | aCEA_scFv-CCD51-BSRS6-XTEN576- | 847 | aCEA_scFv-CCD51-BSRS6-XTEN576- |
| | DM1 | | MMAE |
| 416 | aEpCAM_scFv-CCD51-BSRS6-XTEN576- | 848 | aEpCAM_scFv-CCD51-BSRS6-XTEN576- |
| | DM1 | | MMAE |
| 417 | aHER2_scFv-CCD1-BSRS6-XTEN576- | 849 | aHER2_scFv-CCD1-BSRS6-XTEN576- |
| | DM1 | | MMAE |
| 418 | Folate-CCD1-BSRS6-XTEN576-DM1 | 850 | Folate-CCD1-BSRS6-XTEN576-MMAE |
| 419 | aCEA_scFv-CCD1-BSRS6-XTEN576- | 851 | aCEA_scFv-CCD1-BSRS6-XTEN576-MMAE |
| | DM1 | | |
| 420 | aEpCAM_scFv-CCD1-BSRS6-XTEN576- | 852 | aEpCAM_scFv-CCD1-BSRS6-XTEN576- |
| | DM1 | | MMAE |
| 421 | aHER2_scFv-CCD7-BSRS6-XTEN576- | 853 | aHER2_scFv-CCD7-BSRS6-XTEN576- |
| | DM1 | | MMAE |
| 422 | Folate-CCD7-BSRS6-XTEN576-DM1 | 854 | Folate-CCD7-BSRS6-XTEN576-MMAE |
| 423 | aCEA_scFv-CCD7-BSRS6-XTEN576- | 855 | aCEA_scFv-CCD7-BSRS6-XTEN576-MMAE |
| | DM1 | | |
| 424 | aEpCAM_scFv-CCD7-BSRS6-XTEN576- | 856 | aEpCAM_scFv-CCD7-BSRS6-XTEN576- |
| | DM1 | | MMAE |
| 425 | aHER2_scFv-CCD12-BSRS6-XTEN576- | 857 | aHER2_scFv-CCD12-BSRS6-XTEN576- |
| | DM1 | | MMAE |
| 426 | Folate-CCD12-BSRS6-XTEN576-DM1 | 858 | Folate-CCD12-BSRS6-XTEN576-MMAE |
| 427 | aCEA_scFv-CCD12-BSRS6-XTEN576- | 859 | aCEA_scFv-CCD12-BSRS6-XTEN576- |
| | DM1 | | MMAE |
| 428 | aEpCAM_scFv-CCD12-BSRS6-XTEN576- | 860 | aEpCAM_scFv-CCD12-BSRS6-XTEN576- |
| | DM1 | | MMAE |
| 429 | aHER2_scFv-CCD16-BSRS6-XTEN576- | 861 | aHER2_scFv-CCD16-BSRS6-XTEN576- |
| | DM1 | | MMAE |
| 430 | Folate-CCD16-BSRS6-XTEN576-DM1 | 862 | Folate-CCD16-BSRS6-XTEN576-MMAE |
| 431 | aCEA_scFv-CCD16-BSRS6-XTEN576- | 863 | aCEA_scFv-CCD16-BSRS6-XTEN576- |
| | DM1 | | MMAE |
| 432 | aEpCAM_scFv-CCD16-BSRS6-XTEN576- | 864 | aEpCAM_scFv-CCD16-BSRS6-XTEN576- |
| | DM1 | | MMAE |

*Provides the description of the individual components of the targeted conjugate compositions components
¹Provides the name of the targeting moiety wherein each TM other than folate comprises the VH and VL sequences of the indicated antibody as listed in Table 19 linked by a linker of Table 20.
²Provides the name of the cysteine cotaining domain of Table 6
³Provides the name of the PCM sequence of Table 8
⁴Provides the length of the XTEN of Table 10 (e.g., XTEN713 can be an AE713, AF713 or AG713)
⁵Provides the type of drug molecules conjugated to the CCD wherein the number of drug molecules is equal to the number of cysteine residues of the corresponding CCD of the composition

2. Cysteine Containing Domains

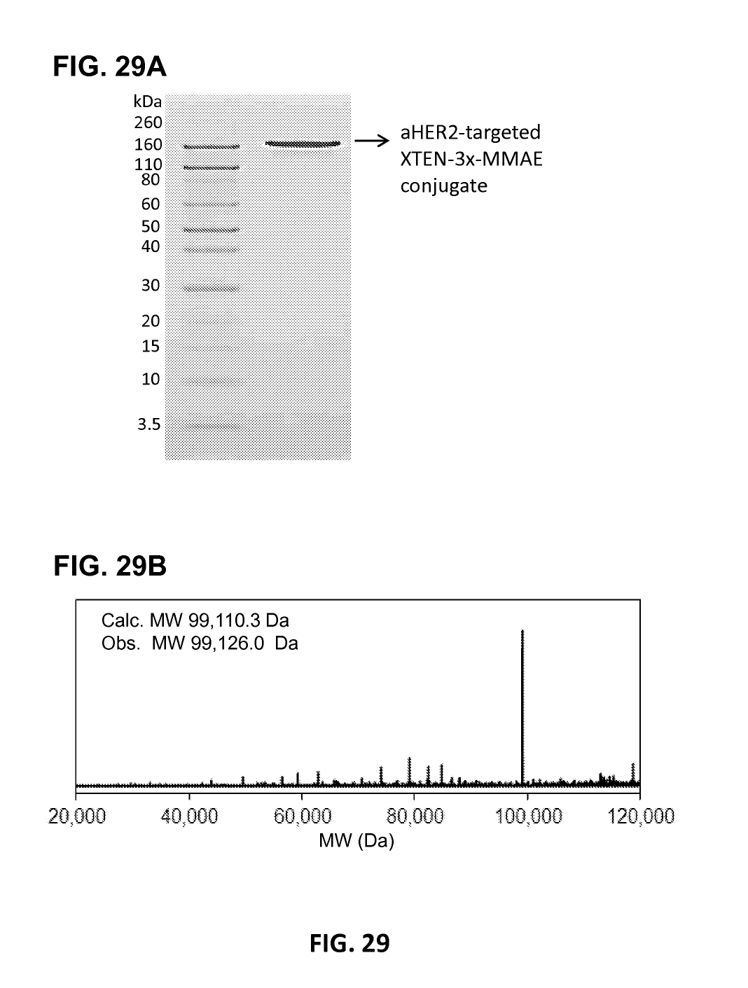

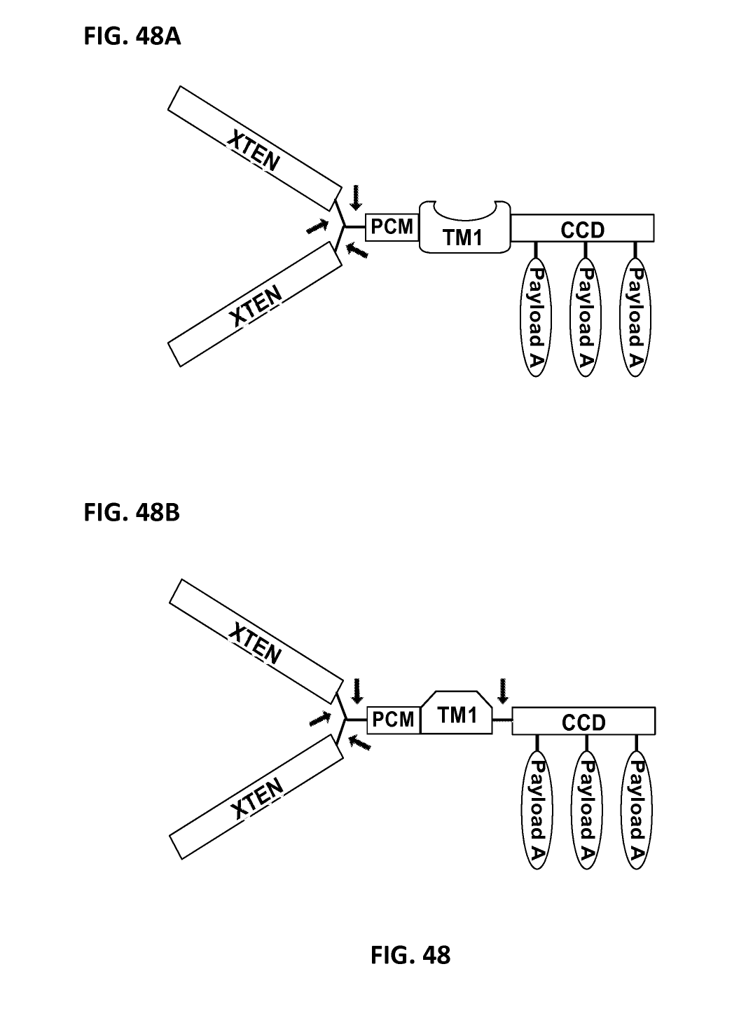

In another aspect, the invention provides polypeptides of short length comprising one or more cysteine residues for the subject compositions to which the drug or biologic payloads described herein are conjugated using cross-linkers (described more fully, below) to link the payloads to the thiol groups of the cysteine residues. In some embodiments, the cysteine containing domains, or “CCD” are polypeptides of relatively short length, and typically comprise at least 6 amino acid residues. In some embodiments, a CCD has between 6 to about 144 amino acids, and between 1 to about 10, or more cysteine residues. Typically, the ratio of cysteine to non-cysteine residues in a CCD is higher than most naturally-occuring peptides and proteins. It is an object of the invention to provide CCD for incorporation into the the subject compositions of the disclosure that comprise targeting moieties, XTEN and, optionally, protease cleavage moieties, in which the fusion protein is specifically configured to locate CCD bearing the linked payload drugs or biologically active proteins in close proximity to the targeting moiety to better ensure that the full number of incorporated payload molecules are delivered to the cell bearing the ligand to which the targeting moiety can bind. While XTEN are not highly prone to proteolytic cleavage in the blood (as demonstrated in the Examples 29 and 48, below, and FIGS. 29 and 48), they are nevertheless susceptible to certain proteases, such as neutrophil elastase, MMP-2, and MMP-9, such that a composition comprising an XTEN administered to a subject is eventually cleaved and degraded by proteolysis over time. In order to optimize the delivery of the intended number of linked payloads to the target tissues, some CCD polypeptides were designed to provide short sequences that have up to 10 cysteine residues interspersed with hydrophilic amino acids. In some embodiments, the invention provides CCD for incorporation into the subject compositions that comprise at least one non-cysteine residue, wherein non-cysteine residues are selected from 3-6 types of amino acids selected the group consisting of glycine (G), alanine (A), serine (S), threonine (T), glutamate (E) and proline (P). In one embodiment, a CCD has 1 cysteine residue and up to 9 non-cysteine residues selected from 3-6 types of amino acids selected from the group consisting of glycine (G), alanine (A), serine (S), threonine (T), glutamate (E) and proline (P). In another embodiment, a CCD of the subject composition has 3 cysteine residues and up to 39 non-cysteine residues selected from the group consisting of 3-6 types of amino acids selected from glycine (G), alanine (A), serine (S), threonine (T), glutamate (E) and proline (P), wherein the cysteine residues can be contiguous or may be separated from another cysteine residue by up to 15 non-cysteine residues. In another embodiment, a CCD of the subject composition has 9 cysteine residues and up to 135 non-cysteine residues selected from the group consisting of glycine (G), alanine (A), serine (S), threonine (T), glutamate (E) and proline (P), wherein no two cysteine residues are contiguous and each cysteine residue may be separated from another cysteine residue by up to 15 non-cysteine residues in the CCD sequence. In another embodiment, a CCD of the subject composition has 1 to 9 cysteine residues and between 6 and 144 total residues (in which the non-cysteine residues are 3-6 types selected from the group consisting of glycine (G), alanine (A), serine (S), threonine (T), glutamate (E) and proline (P)) in which a cysteine residue is located within 2 to 9 residues of the N- or C-terminus of the CCD. In another embodiment, a CCD of the subject composition has 3 to 9 cysteine residues and between 14 and 144 total residues (in which the non-cysteine residues are 3-6 types selected from the group consisting of glycine (G), alanine (A), serine (S), threonine (T), glutamate (E) and proline (P)) and any 2 cysteine residues are separated by no more than 15 non-cysteine residues. In another embodiment, the invention provides CCD for incorporation into the subject compositions having a sequence with at least 90% sequence identity to a sequence selected from the group consisting of the sequences set forth in Table 6. In another embodiment, the invention provides CCD for incorporation into the subject compositions selected from the group consisting of the sequences set forth in Table 6. In another embodiment, the invention provides a fusion protein comprising a CCD having a sequence selected from the group consisting of the sequences set forth in Table 6 fused to a targeting moiety disclosed herein. In another embodiment, the invention provides a fusion protein comprising a CCD having a sequence selected from the group consisting of the sequences set forth in Table 6 fused between a targeting moiety and an XTEN disclosed herein. In another embodiment, the invention provides a fusion protein comprising a CCD having a sequence selected from the group consisting of the sequences set forth in Table 6 fused between a targeting moiety and a PCM disclosed herein. In another embodiment, the invention provides a fusion protein comprising a CCD having a sequence selected from the group consisting of the sequences set forth in Table 5, a targeting moiety, a PCM, and an XTEN disclosed herein. In another embodiment, the invention provides a fusion protein comprising a CCD having a sequence selected from the group consisting of the sequences set forth in Table 6, a PCM, and an XTEN disclosed herein, together with a targeting moiety conjugated to the N- or C-terminus of the CCD.

It is another object of the invention to provide CCD for incorporation into the subject compositions to enhance the ability to recover molecules of the compositions after a conjugation reaction wherein the composition has the full number of intended of payload drug or biologic molecules conjugated to each of the cysteine residues incorporated into the CCD. The invention takes advantage of the surprising discovery that in HPLC analyses drug conjugates of CCD-XTEN fusion proteins provide signically improved peak separation between conjugates having different numbers of drug molcules. The difference can be seen in reaction products comparing XTEN with incorporated cysteine residues spread evenly across the sequence (e.g., the cysteine engineered XTEN of Table 11) versus a fusion protein of an XTEN of Table 10 fused with a CCD with the same number of amino acids as the cysteine-engineer XTEN. The respective polypeptides were subjected to a conjugation reaction to link a given payload to the cysteines, and upon HPLC analysis, the reaction product of the fusion protein of the XTEN and the CCD had significantly greater peak separation with respect to the peak corresponding to the fully-conjugated reaction product relative to the peak corresponding to the underconjugated reaction product that was the closest to the fully conjugated reaction product peak, as compared to the separation of the corresponding peaks of the reaction products of the cysteine-containing XTEN conjugate. Stated differently, compositions comprising CCD with conjugated payload drug or biologically active proteins incorporated into a targeted conjugate composition are capable of achieving greater separation between peaks of the heterogenous conjugation reaction products on reversed-phase HPLC chromatography than the reaction products of a composition wherein the cysteine residues are more evenly distributed across the length of an XTEN of corresponding length not comprising a CCD.

The separation between the peak of the fully conjugated product to the next nearest under-conjugated product can be mathematically defined. As used herein, “Peak Separation” is defined as follows:

Peak Separation=(t_{R2}−t_{R1})/FWHM
wherein
   t_{R2}: retention time of the fully conjugated product peak by reverse phase HPLC;
   t_{R1}: retention time of the underconjugated peak that is closest to the fully conjugated product peak by reverse phase HPLC; and
   FWHM: full width at half maximum of the fully conjugated product peak
wherein the reversed-phase HPLC chromatography conditions are as follows:
   HPLC column is C4-HPLC column (Vydac, catalog number: 214TP5415 Vydac C4)
   Elution Method: 5-50% Buffer B in 45 minutes, 1 ml/min
   Buffer A: 0.1% TFA in H₂O
   Buffer B: 0.1% TFA in acetonitrile

In some embodiments, the invention provides targeted conjugate compositions wherein upon the conjugation between a drug molecule and the cysteine residues of the CCD of the fusion protein, a heterogeneous population of conjugate products is obtained wherein fully conjugated CCD-drug conjugate product is capable of achieving a Peak Separation ≥6 wherein: a) the fusion protein comprises a polypeptide having 600 or more cumulative amino acid residues comprising a CCD with between 3 to 9 cysteine residues; b) the heterogeneous conjugate products have a mixture of at least 1, 2, and 3 or more payloads linked to the CCD; and c) the heterogeneous population of conjugation products are analyzed under reversed-phase HPLC chromatography conditions. In one embodiment of the foregoing, the CCD of the fusion protein is a sequence of Table 6 having 3 cysteine residues and the fusion protein has at least 800 cumulative amino acid residues. In another embodiment of the foregoing, CCD of the fusion protein is a sequence of Table 6 having 9 cysteine residues and the fusion protein has at least 800 cumulative amino acid residues.

TABLE 6
Cysteine Containing Domains (CCD)
for conjugation to drug payload

| CCD | | AA | | SEQ | | SEQ |
|---|---|---|---|---|---|---|
| Desig- | Number | between | Amino Acid | ID | | ID |
| nation | of Cys | Cys | Sequence | NO: | DNA Sequence | NO: |
| CCD1 | 3 | 15 | GSPGAGSCAGSPTS | 26 | GGcTCTCCAggtgcAGGTA | 77 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD2 | 3 | 10 | GSPGPAGCGAPGPS | 27 | GGcTCTCCAGGTCCGGCCG | 78 |
| | | | GGPTCTGSTSATPG | | GTtgcGGTGCACCGGGCCC | |
| | | | ACSA | | GAGTGGTGGTCCTACAtgt | |
| | | | | | ACCGGCAGTACCAGCGCAA | |
| | | | | | CACCTGGTGCAtgcAGCGC | |
| | | | | | C | |
| CCD3 | 3 | 15 | GSPGAGSCTETSPS | 28 | GGcTCTCCAggtgcAGGTA | 79 |
| | | | TPTESPEAGCSGSG | | GCtgtACGGAAACCAGCCC | |
| | | | SPESPSGTEASCTS | | GAGCACCCCGACCGAGTCC | |
| | | | | | CCGGAAGCGGGCtgcAGCG | |
| | | | | | GTAGCGGCAGCCCGGAGAG | |
| | | | | | CCCGAGCGGTACCGAGGCG | |
| | | | | | AGCtgtACGTCC | |
| CCD4 | 3 | 10 | GSPGAGSCTETSPS | 29 | GGcTCTCCAggtgcAGGTA | 80 |
| | | | TPTSCPEAGSGSGS | | GCtgtACGGAAACCAGCCC | |
| | | | PCSP | | GAGCACCCCGACCTCCtgc | |
| | | | | | CCGGAAGCGGGCAGCGGTA | |
| | | | | | GCGGCAGCCCGtgtAGCCC | |
| | | | | | G | |
| CCD5 | 3 | 7 | GSPGAGSCTETSPS | 30 | GGcTCTCCAggtgcAGGTA | 81 |
| | | | TCPTESPEACGS | | GCtgtACGGAAACCAGCCC | |
| | | | | | GAGCACCtgcCCGACCGAG | |
| | | | | | TCCCCGGAAGCGtgtGGCA | |
| | | | | | GC | |
| CCD6 | 3 | 3 | GSPGAGSCTETCSP | 31 | GGcTCTCCAggtgcAGGTA | 82 |
| | | | SCTP | | GCtgtACGGAAACCtgcAG | |
| | | | | | CCCGAGCtgtACCCCG | |
| CCD7 | 3 | 3 | GAPCGAGCAGPCGP | 32 | GGcgCTCCAtgTggtgcAG | 83 |
| | | | | | GTtgcGCaGGTCCAtgtGG | |
| | | | | | CCCG | |
| CCD8 | 3 | 1 | GSPGAGSCTCTCSP | 33 | GGcTCTCCAggtgcAGGTA | 84 |
| | | | | | GCtgtACGtgcACCtgtAG | |
| | | | | | CCCG | |
| CCD9 | 3 | 0 | GSPGAGSCCCTE | 34 | GGcTCTCCAggtgcAGGTA | 85 |
| | | | | | GCtgttgctgtACGGAA | |
| CCD10 | 3 | 7 | GSPCGAGESTTCSP | 35 | GGcTCTCCAtgTggtgcAG | 86 |
| | | | STPTSCPE | | GTGAGAGCACGACCtgcAG | |
| | | | | | CCCGAGCACCCCGACCTCC | |
| | | | | | tgtCCGGAA | |
| CCD11 | 3 | 3 | GSPCGAGCSTTCSP | 36 | GGcTCTCCAtgTggtgcAG | 87 |
| | | | | | GTtgcAGCACGACCtgtAG | |
| | | | | | CCCG | |
| CCD12 | 9 | 15 | GSPGAGSCAGSPTS | 37 | GGcTCTCCAggtgcAGGTA | 88 |
| | | | TEEGTSESACSPEG | | GCtgcGCTGGTAGCCCAAC | |
| | | | PGTSTEPSEGSCGP | | CTCTACCGAAGAAGGTACC | |
| | | | SPAGSPTSTEEGTC | | TCTGAATCCGCTtgTTCCC | |
| | | | TEPSEGSAPGTSEP | | CAGAAGGTCCTGGTACTAG | |
| | | | TCSGSAPGTSESAT | | CACTGAGCCAAGCGAAGGT | |
| | | | PESCGPSEPATSGS | | TCTtgTGGCCCATCCCCGG | |
| | | | ETPGSCAPTSGSET | | CAGGTAGCCCTACCTCTAC | |
| | | | PGSPAGSCTSTEEG | | CGAAGAGGGCACTtGCACC | |
| | | | TSESATPESCGPTE | | GAACCATCTGAGGGTTCCG | |
| | | | SASG | | CTCCTGGCACCTCCGAACC | |
| | | | | | GACTtgcTCCGGCAGTGCT | |
| | | | | | CCGGGTACTTCCGAAAGCG | |
| | | | | | CAACTCCGGAATCCtGCGG | |
| | | | | | TCCTTCTGAGCCTGCTACT | |
| | | | | | TCCGGCTCTGAAACTCCAG | |
| | | | | | GTAGCtgtGCGCCAACTTC | |
| | | | | | TGGTTCTGAAACTCCAGGT | |
| | | | | | TCACCGGCGGGTAGCtgcA | |
| | | | | | CGAGCACGGAGGAAGGTAC | |
| | | | | | CTCTGAGTCGGCCACTCCT | |
| | | | | | GAGTCCtGTGGCCCGACGG | |
| | | | | | AAagcgcctctGGC | |
| CCD13 | 9 | 10 | SGTASSSCPGSSTP | 38 | | |
| | | | SGATCGSPGTPGSG | | | |
| | | | TCASSSPGSSTPCS | | | |
| | | | GATGSPGSSCTPSG | | | |
| | | | ATGSPGCSSPSAST | | | |
| | | | GTGCPGSSPSASTG | | | |
| | | | CTGPGASPGTSCST | | | |
| | | | GSPGTP | | | |
| CCD14 | 9 | 15 | GSEPATSCGSETPG | 39 | | |
| | | | TSESATPESCGPGS | | | |
| | | | EPATSGSETPGCSP | | | |
| | | | AGSPTSTEEGTSTC | | | |
| | | | PSEGSAPGSEPATS | | | |
| | | | GCSETPGSEPATSG | | | |
| | | | SETCPGSEPATSGS | | | |
| | | | ETPGTCSTEPSEGS | | | |
| | | | APGTSESCATPESG | | | |
| | | | PGSEPATSGCSETP | | | |
| | | | GTST | | | |
| CCD15 | 9 | 10 | GSEPATSCGSETPG | 40 | | |
| | | | TSESCATPESGPGS | | | |
| | | | PCATSGSETPGSCP | | | |
| | | | AGSPTSTGTCSTEP | | | |
| | | | SEGSAPCGSEPATS | | | |
| | | | GSTCPGSEPATSGS | | | |
| | | | CTPGSEPATSGCSE | | | |
| | | | TPGTST | | | |
| CCD16 | 9 | 7 | GSPGAGSCTETSPS | 41 | GGcTCTCCAggtgcAGGTA | 89 |
| | | | TCPTESPEACGSGS | | GCtgtACGGAAACCAGCCC | |
| | | | GSPCSPSGTEACST | | GAGCACCtgcCCGACCGAG | |
| | | | SGSEGCSPSSTAPC | | TCCCCGGAAGCGtgtGGCA | |
| | | | GPTETEGCTTSSGP | | GCGGTAGCGGCAGCCCGtg | |
| | | | PCPESATSEG | | cAGCCCGAGCGGTACCGAG | |
| | | | | | GCGtgtAGCACGTCCGGCT | |
| | | | | | CGGAAGGTtgcTCTCCGTC | |
| | | | | | CTCCACGGCACCGtgtGGC | |
| | | | | | CCGACCGAAACCGAGGGCt | |
| | | | | | gcACGACCAGCAGCGGTCC | |
| | | | | | GCCGtgtCCGGAGAGCGCT | |
| | | | | | ACCTCCGAGGGT | |
| CCD17 | 3 | 15 | GSPGAGSCAGSPTS | 42 | GGcTCTCCAggtgcAGGTA | 90 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD18 | 3 | 15 | GSPGAGSCAGSPTS | 43 | GGcTCTCCAggtgcAGGTA | 91 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD19 | 3 | 15 | GSPGAGSCAGSPTS | 44 | GGcTCTCCAggtgcAGGTA | 92 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD20 | 3 | 15 | GSPGAGSCAGSPTS | 45 | GGcTCTCCAggtgcAGGTA | 93 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD21 | 3 | 3 | GAPCGAGCAGPCGP | 46 | GGcgCTCCAtgTggtgcAG | 94 |
| | | | | | GTtgcGCaGGTCCAtgtGG | |
| | | | | | CCCG | |
| CCD22 | 3 | 15 | GSPGAGSCAGSPTS | 47 | | |
| | | | TEEGTSESACSPEG | | | |
| | | | PGTSTEPSEGSCGG | | | |
| CCD23 | 3 | 15 | GSPGAGSCAGSPTS | 48 | GGcTCTCCAggtgcAGGTA | 95 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD24 | 3 | 15 | GSPGAGSCAGSPTS | 49 | GGcTCTCCAggtgcAGGTA | 96 |
| | | | TEEGTSESACSPEG | | GCtgcGCTGGTAGCCCAAC | |
| | | | PGTSTEPSEGSCGG | | CTCTACCGAAGAAGGTACC | |
| | | | | | TCTGAATCCGCTtgTTCCC | |
| | | | | | CAGAAGGTCCTGGTACTAG | |
| | | | | | CACTGAGCCAAGCGAAGGT | |
| | | | | | TCTtgTGGCggt | |
| CCD25 | 3 | 15 | GSPGAGSCAGSPTS | 50 | | |
| | | | TEEGTSESACSPEG | | | |
| | | | PGTSTEPSEGSCGG | | | |
| CCD26 | 3 | 15 | GSPGAGSCAGSPTS | 51 | | |
| | | | TEEGTSESACSPEG | | | |
| | | | PGTSTEPSEGSCGG | | | |
| CCD27 | 3 | 15 | GAPGSPACGSPTST | 52 | | |
| | | | EEGTSESATCPESG | | | |
| | | | PGSEPATSGSTCPA | | | |
| CCD28 | 3 | 15 | TEPSEGSCAPGSPA | 53 | | |
| | | | GSPTSTEEGCTSES | | | |
| | | | ATPESGPGSEPCAT | | | |
| CCD29 | 3 | 15 | PAGSPTSCTEEGTS | 54 | | |
| | | | TEPSEGSAPCGTSE | | | |
| | | | SATPESGPGSPCAT | | | |
| CCD30 | 3 | 15 | SEPATSGCSETPGT | 55 | | |
| | | | SESATPESGCPGTS | | | |
| | | | TEPSEGSAPGSCPA | | | |
| CCD31 | 3 | 15 | GAPSPSACSTGTGP | 56 | | |
| | | | GTPGSGTASCSSPG | | | |
| | | | SSTPSGATGSPCGP | | | |
| CCD32 | 3 | 15 | GPGTPGSCGTASSS | 57 | | |
| | | | PGSSTPSGACTGSP | | | |
| | | | GSSPSASTGTGCPG | | | |
| CCD33 | 3 | 15 | SPSASTGCTGPGAS | 58 | | |
| | | | PGTSSTGSPCGTPG | | | |
| | | | SGTASSSPGSSCTP | | | |
| CCD34 | 3 | 15 | SASTGTGCPGASPG | 59 | | |
| | | | TSSTGSPGTCPGSG | | | |
| | | | TASSSPGSSTPCSG | | | |
| CCD35 | 3 | 15 | GTSTPESCGSASPG | 60 | | |
| | | | TSPSGESSTCAPGT | | | |
| | | | SPSGESSTAPGCST | | | |
| CCD36 | 3 | 15 | AESPGPGCSTSESP | 61 | | |
| | | | SGTAPGSTSCSTAE | | | |
| | | | SPGPGTSPSGSCST | | | |
| CCD37 | 3 | 15 | GSTSSTACSPGPGS | 62 | | |
| | | | TSSTAESPGCPGST | | | |
| | | | SESPSGTAPGSCTS | | | |
| CCD38 | 3 | 15 | ESSTAPGCSTSESP | 63 | | |
| | | | SGTAPGSTSCSPSG | | | |
| | | | TAPGTSPSGESCST | | | |
| CCD39 | 3 | 3 | GSPCATSCGSTCPG | 64 | | |
| CCD40 | 3 | 3 | GTSCSATCPESCGP | 65 | | |
| CCD41 | 3 | 3 | GTSCTEPCSEGCSA | 66 | | |
| CCD42 | 3 | 3 | GSTCSESCPSGCTA | 67 | | |
| CCD43 | 3 | 3 | GTSCTPSCGSACSP | 68 | | |
| CCD44 | 3 | 3 | GTSCPSGCSSTCAP | 69 | | |
| CCD45 | 3 | 3 | GSTCSSTCAESCPG | 70 | | |
| CCD46 | 3 | 3 | GTPCGSGCTASCSS | 71 | | |
| CCD47 | 3 | 3 | GSSCTPSCGATCGS | 72 | | |
| CCD48 | 3 | 3 | GSSCPSACSTGCTG | 73 | | |
| CCD49 | 3 | 3 | GASCPGTCSSTCGS | 74 | | |
| CCD50 | 1 | NA | GSPGAGSCAG | 75 | | |
| CCD51 | 1 | NA | GAPCGA | 76 | | |

3. Peptidic Cleavage Moieties

In one aspect, the invention provides targeted conjugate compositions comprising one or more peptidic cleavage moieties (PCM) that are a substrate for a protease associated with a target tissue in a subject; non-limiting examples of which are a cancer, tumor, or tissues or organs involved in an inflammatory response. It is an object of the invention to provide peptidic cleavage moities (PCM) specifically configured for use in targeted conjugate compositions comprising payloads such that the payloads (with or without some portion of an XTEN sequence) of the compositions, or payloads linked to TM (with or without some portion of an XTEN sequence), are released from the composition when the composition comprising the PCM is in proximity with the targeted tissue-associated protease. The design of the targeted conjugate compositions is such that the resulting released component, comprising the TM and/or the payload have an enhanced ability to attach to or to penetrate into the target tissue; whether by the reduced molecular mass of the resulting fragment or by reduced steric hindrence by the flanking bulky XTEN that is cleaved away.

Stroma in human carcinomas consists of extracellular matrix and various types of non-carcinoma cells such as leukocytes, endothelial cells, fibroblasts, and myofibroblasts. The tumor-associated stroma actively supports tumor growth by stimulating neo-angiogenesis, as well as proliferation and invasion of apposed carcinoma cells. Stromal fibroblasts, often referred to as cancer-associated fibroblasts (CAF), have a particularly important role in tumor progression due to their ability to dynamically modify the composition of the extracellular matrix (ECM), thereby facilitating tumor cell invasion and subsequent metastatic colonization. In particular, it is known in the art that proteases are important components that contribute to malignant progression, including tumor angiogenesis, invasion, extracellular matrix remodeling, and metastasis, where proteases function as part of an extensive multidirectional network of proteolytic interactions.

As a requirement of malignant tumours is their ability to acquire a vasculature system in order to penetrate into surrounding normal tissues and disseminate to distant sites, the tumor relies heavily upon the increased expression of extracellular endoproteases from multiple enzymatic classes; e.g., the metalloproteases (MMP) and serine, threonine, cysteine and aspartic proteases. The role of proteases are not limited to tissue invasion and angiogenesis, however. These enzymes also have major roles in growth factor activation, cellular adhesion, cellular survival and immune surveillance. For example, MMPs are able to impact in vivo on tumour cell behaviour as a consequence of their ability to cleave growth factors, cell surface receptors, cell adhesion molecules, or chemokines. Collectively, the actions of tumor-associated proteases represent a significant force in the phenotypic evolution of cancer.

Considering the differential expression of many such proteolytic enzymes between normal and tumour tissue, this differential expression can be utilized as a means to semi-selectively activate or alter chemotherapeutic agents that are in proximity to or are colocalized with a tumor. As used herein, “colocalized” means that the protease is in highest concentration adjacent to or within a tumor and the concentration diminishes as the distance from the tumor increases. In this respect, the serine and metalloproteases are candidates for targeted, differential drug delivery due to both their elevated activity in the extracellular tumour environment and their ability to selectively and specifically cleave short peptide sequences. Specifically, the increased endoprotease activity within tumours relative to non-diseased tissue can be harnessed to activate prodrug compounds comprising specific peptide sequences and having potent anticancer therapeutics that are subsequently released, resulting in high levels of the active agent at the tumour and low or negative drug levels in normal healthy tissues. As a consequence of the selective delivery of such prodrug cancer therapeutics, there is both a concommitant reduction in the required activity of these agents and reduced toxicity against normal tissues, including liver, heart and bone marrow, thereby greatly improving the therapeutic index of such compounds.

In some embodiments, the invention comprises targeted conjugate compositions comprising PCM wherein when the composition is cleaved by the targeted tissue-associated protease, releasing a fragment comprising the payload, the fragment comprising the payload is capable of penetrating within said tissue to a concentration that is at least 2-fold, or at least 3-fold, or at least 4-fold, or at least 5-fold greater compared to the composition not comprising the PCM. In other embodiments, the invention comprises targeted conjugate compositions comprising PCM wherein when the composition is cleaved by the targeted tissue-associated protease, releasing a released targeted composition fragment comprising the payload and the TM, the released targeted composition is capable of penetrating within said tissue at a rate that is at least 2-fold, or at least 3-fold, or at least 4-fold, or at least 5-fold greater compared to a corresponding composition not comprising the PCM. In one embodiment of the foregoing, the released targeted composition fragment, after its release, has a resulting molecular weight that is at least 4-fold, at least 5-fold, at least 6-fold, at least 7-fold, at least 8-fold, at least 10-fold less than the intact targeted conjugate composition that is not cleaved by the protease. In another embodiment of the foregoing, the released targeted composition, after its release, has a resulting hydrodynamic radius that is at least 4-fold, at least 5-fold, at least 6-fold, at least 7-fold, at least 8-fold, at least 10-fold less than the intact targeted conjugate composition that is not cleaved by the protease. It is specifically contemplated that in the subject targeted conjugate compostion embodiments, the cleavage by the tissue-associated protease results in a fragment comprising the payload that is able to more effectively penetrate the tissue, such as a tumor, because of the reduced size of the fragment relative to the intact composition, resulting in a pharmacologic effect of the payload within said tissue or cell. It is also specifically contemplated that the PCM of the targeted conjugate compositions are designed for use in compositions intended to target specific tissues with a specific protease known to be produced by that target tissue or cell. In one embodiment, the PCM of the targeted conjugate composition comprises an an amino acid sequence that is a substrate for an extracellular protease secreted by the target tissue, including but not limited to the proteases of Table 7. In another embodiment, the PCM of the targeted conjugate composition comprises an an amino acid sequence that is a substrate for an extracellular protease secreted by the target tissue, including but not limited to the group of sequences set forth in Table 8. In another embodiment, the PCM comprises an amino acid sequence that is a substrate for a cellular protease located within a cell, including but not limited to the proteases of Table 7. In another embodiment, the PCM comprises an amino acid sequence sequence that is a substrate for a protease associated with a tissue that is a cancer. In another embodiment, the PCM comprises an amino acid sequence sequence that is a substrate for a protease associated with a cancerous tumor. In another embodiment, the PCM comprises an amino acid sequence sequence that is a substrate for a protease associated with a cancer such as a leukemia. In another embodiment, the PCM comprises an amino acid sequence sequence that is a substrate for a protease associated with an inflammatory tissue.

In one embodiment, the PCM of the targeted conjugate composition is a substrate for at least one protease selected from the group consisting of the group of proteases set forth in Table 7. In some embodiments, the PCM is a substrate for at least one protease selected from the group consisting of metalloproteinases, cysteine proteases, aspartate proteases, and serine proteases. In another embodiment, the PCM is a substrate for one or more proteases selected from the group consisting of meprin, neprilysin (CD10), PSMA, BMP-1, A disintegrin and metalloproteinases (ADAMs), ADAMS, ADAMS, ADAM10, ADAM12, ADAM15, ADAM17 (TACE), ADAM19, ADAM28 (MDC-L), ADAM with thrombospondin motifs (ADAMTS), ADAMTS1, ADAMTS4, ADAMTS5, MMP-1 (Collagenase 1), MMP-2 (Gelatinase A), MMP-3 (Stromelysin 1), MMP-7 (Matrilysin 1), MMP-8 (Collagenase 2), MMP-9 (Gelatinase B), MMP-10 (Stromelysin 2), MMP-11(Stromelysin 3), MMP-12 (Macrophage elastase), MMP-13 (Collagenase 3), MMP-14 (MT1-MMP), MMP-15 (MT2-MMP), MMP-19, MMP-23 (CA-MMP), MMP-24 (MT5-MMP), MMP-26 (Matrilysin 2), MMP-27 (CMMP), Legumain, Cathepsin B, Cathepsin C, Cathepsin K, Cathepsin L, Cathepsin S, Cathespin X, Cathepsin D, Cathepsin E, Secretase, urokinase (uPA), Tissue-type plasminogen activator (tPA), plasmin, thrombin, prostate-specific antigen (PSA, KLK3), human neutrophil elastase (HNE), Elastase, Tryptase, Type II transmembrane serine proteases (TTSPs), DESC1, Hepsin (HPN), Matriptase, Matriptase-2, TMPRSS2, TMPRSS3, TMPRSS4 (CAP2), Fibroblast Activation Protein (FAP), kallikrein-related peptidase (KLK family), KLK4, KLK5, KLK6, KLK7, KLK8, KLK10, KLK11, KLK13, and KLK14. In some embodiments, the PCM is a substrate for an ADAM17. In some embodiments, the PCM is a substrate for a BMP-1. In some embodiments, the PCM is a substrate for a cathepsin. In some embodiments, the PCM is a substrate for a cysteine protease. In some embodiments, the PCM is a substrate for a HtrAl. In some embodiments, the PCM is a substrate for a legumain. In some embodiments, the PCM is a substrate for a MT-SP1. In some embodiments, the PCM is a substrate for a metalloproteinase. In some embodiments, the PCM is a substrate for a neutrophil elastase. In some embodiments, the PCM is a substrate for a thrombin. In some embodiments, the PCM is a substrate for a Type II transmembrane serine protease (TTSP). In some embodiments, the PCM is a substrate for TMPRSS3. In some embodiments, the PCM is a substrate for TMPRSS4. In some embodiments, the PCM is a substrate for uPA. In one embodiment, the PCM comprises a cleavage sequence selected from the group of sequences set forth in Table 8. In another embodiment, the PCM of the cleavage conjugate compostion comprises a first cleavage sequence and a second cleavage sequence different from said first cleavage sequence wherein each sequence is selected from the group of sequences set forth in Table 8 and the first and the second cleavage sequences are linked to each other by 1 to 6 amino acids selected from glycine, serine, alanine, and threonine. In another embodiment, the PCM of the cleavage conjugate compostion comprises a first cleavage sequence, a second cleavage sequence different from said first cleavage sequence, and a third cleavage sequence wherein each sequence is selected from the group of sequences set forth in Table 8 and the first and the second and the third cleavage sequences are linked to each other by 4 to 6 amino acids selected from glycine, serine, alanine, and threonine. In other embodiments, the invention provides targeted conjugate compositions comprising one, two, or three PCM. It is specifically intended that the multiple PCM of the targeted conjugate compositions can be concatenated to form a universal sequence that can be cleaved by multiple proteases. It is contemplated that such compositions would be more readily cleaved by diseased target tissues that express multiple proteases, with the result that the resulting fragments bearing the TM and/or the payload drug(s) would more readily penetrate the target tissue and exert the pharmacologic effect of the payload drug(s).

TABLE 7
Proteases of Target Tissues.

| Class of Proteases | Protease |
|---|---|
| Metalloproteinases | Meprin |
| | Neprilysin (CD10) |
| | PSMA |
| | BMP-1 |
| | A disintegrin and metalloproteinases (ADAMs) |
| | ADAM8 |
| | ADAM9 |
| | ADAM10 |
| | ADAM12 |
| | ADAM15 |
| | ADAM17 (TACE) |
| | ADAM19 |
| | ADAM28 (MDC-L) |
| | ADAM with thrombospondin motifs (ADAMTS) |
| | ADAMTS1 |
| | ADAMTS4 |
| | ADAMTS5 |
| | Matrix Metalloproteinases (MMPs) |
| | MMP-1 (Collagenase 1) |
| | MMP-2 (Gelatinase A) |
| | MMP-3 (Stromelysin 1) |
| | MMP-7 (Matrilysin 1) |
| | MMP-8 (Collagenase 2) |
| | MMP-9 (Gelatinase B) |
| | MMP-10 (Stromelysin 2) |
| | MMP-11(Stromelysin 3) |
| | MMP-12 (Macrophage elastase) |
| | MMP-13 (Collagenase 3) |
| | MMP-14 (MT1-MMP) |
| | MMP-15 (MT2-MMP) |
| | MMP-19 |
| | MMP-23 (CA-MMP) |
| | MMP-24 (MT5-MMP) |
| | MMP-26 (Matrilysin 2) |
| | MMP-27 (CMMP) |
| Cysteine Proteases | Legumain |
| | Cysteine Cathepsins |
| | Cathepsin B |
| | Cathepsin C |
| | Cathepsin K |
| | Cathepsin L |
| | Cathepsin S |
| | Cathespin X |
| Aspartate Proteases | Cathepsin D |
| | Cathepsin E |
| | Secretase |
| Serine Proteases | Urokinase (uPA) |
| | Tissue-type plasminogen activator (tPA) |
| | Plasmin |
| | Thrombin |
| | Prostate-specific antigen (PSA, KLK3) |
| | Human neutrophil elastase (HNE) |
| | Elastase |
| | Tryptase |
| | Type II transmembrane serine proteases (TTSPs) |
| | DESC1 |
| | Hepsin (HPN) |
| | Matriptase |
| | Matriptase-2 |
| | TMPRSS2 |
| | TMPRSS3 |
| | TMPRSS4 (CAP2) |
| | Fibroblast Activation Protein (FAP) |
| | kallikrein-related peptidase (KLK family) |
| | KLK4 |
| | KLK5 |
| | KLK6 |
| | KLK7 |
| | KLK8 |
| | KLK10 |
| | KLK11 |
| | KLK13 |
| | KLK14 |

In certain embodiments, the invention provides PCM compositions intended for use in the subject targeted conjugate compositions comprising at least a first cleavage sequence selected from the group of sequences set forth in Table 8. In some embodiments, the PCM composition sequences are designed with certain properties in mind, including that 1) the nucleic acid encoding the sequences can be readily linked to or within a nucleic acid sequence encoding an XTEN or targeting moiety, resulting in a sequence that can be expressed and recovered as a fusion protein; and 2) the resulting fusion protein can serve as a substrate for a target tissue protease described herein. In one embodiment, the PCM exhibits at least about 90% identity, or at least about 93% identity, or at least about 94% identity, or at least about 95% identity, or at least about 96% identity, or at least about 97% identity, or at least about 98% identity, or at least about 99% identity, or is identical to a peptidyl cleavage sequence selected from the group consisting of the sequences set forth in Table 8.

TABLE 8
Sequences of Peptidyl Cleavage Moieties (PCM)

| PCM | Protease Acting | Exemplary | SEQ ID | Cleavage | SEQ ID |
|---|---|---|---|---|---|
| Designation | Upon Sequence | Cleavage Sequence | NO: | Sequences* | NO: |
| BSRS1 | MMP-2, 7, 9, 14, | LSGR↓SDN↓HSPLG↓LAGS | 97 | | |
| | matriptase, uPA, | | | | |
| | legumain | | | | |
| BSRS2 | MMP-2, 7, 9, 14, | SPLG↓LAGSLSGR↓SDN↓H | 98 | | |
| | matriptase, uPA, | | | | |
| | legumain | | | | |
| BSRS3 | MMP-2, 7, 9, 14, | SPLG↓LSGR↓SDN↓H | 99 | | |
| | matriptase, uPA, | | | | |
| | legumain | | | | |
| BSRS4 | MMP-2, 7, 9, 14, | LAGR↓SDN↓HSPLG↓LAGS | 100 | | |
| | matriptase, uPA, | | | | |
| | legumain | | | | |
| BSRS5 | MMP-2, 7, 9, 14, | LAGR↓SDN↓HVPLS↓LSMG | 101 | | |
| | matriptase, uPA, | | | | |
| | legumain | | | | |
| BSRS6 | MMP-2, 7, 9, 14, | LAGR↓SDN↓HEPLE↓LVAG | 102 | | |
| | matriptase, uPA, | | | | |
| | legumain | | | | |
| RS1 | MMP-2, 7, 9, 14 | SPLG↓LAGS | 103 | | |
| RS2 | MMP-2, 7, 9, 14, | GPLG↓LAR↓G | 104 | | |
| | matriptase, uPA, | | | | |
| | legumain | | | | |
| RS3 | Matriptase, uPA, | LSGR↓SDN↓H | 105 | | |
| | legumain | | | | |
| RS4 | MMP-2, 14 | GTAH↓LMGG | 106 | | |
| RS5 | MMP-14 | RIGS↓LRTA | 107 | | |
| RS6 | MMP-14 | RIGA↓LRTA | 108 | | |
| RS7 | MMP-14 | RIGW↓LRTA | 109 | | |
| RS8 | MMP-14 | RIGN↓LRTA | 110 | | |
| RS9 | MMP-14 | RIGF↓LRTA | 111 | | |
| RS10 | MMP-14 | RIFF↓LRTA | 112 | | |
| RS11 | MMP-14 | RILF↓LRTA | 113 | | |
| RS12 | MMP-14 | RIYF↓LRTA | 114 | | |
| RS13 | MMP-14 | RIQF↓LRTA | 115 | | |
| RS14 | MMP-14 | EPAA↓LMAG | 116 | | |
| RS15 | MMP-14 | EPAN↓LMAG | 117 | | |
| RS16 | MMP-14 | EPAS↓LMAG | 118 | | |
| RS17 | MMP-14 | EPFH↓LMAG | 119 | | |
| RS18 | MMP-14 | EPWH↓LMAG | 120 | | |
| RS19 | MMP-14 | EPRH↓LMAG | 121 | | |
| RS20 | MMP-7 | VPLS↓LFMG | 122 | | |
| RS21 | MMP-7 | VPLS↓LHMG | 123 | | |
| RS22 | MMP-7 | VPLS↓LQAG | 124 | | |
| RS23 | MMP-2, 7, 9, 14 | VPLS↓LTMG | 125 | | |
| RS24 | MMP-2, 7, 9, 14, | VPLS↓LKMG | 126 | | |
| | matriptase | | | | |
| RS25 | MMP-2, 7, 9, 14 | VPLS↓LSMG | 127 | | |
| RS26 | MMP-7 | VPLS↓LNAG | 128 | | |
| RS27 | MMP-7 | VPLS↓LLMG | 129 | | |
| RS28 | MMP-7 | EPLE↓LPAG | 130 | | |
| RS29 | MMP-2,7, 9, 14 | EPLE↓LAAG | 131 | | |
| RS30 | MMP-2, 7, 9 | EPLE↓LVAG | 132 | | |
| RS31 | MMP-7 | EPLE↓LSAG | 133 | | |
| RS32 | MMP-7 | EPLE↓LDAG | 134 | | |
| RS33 | MMP-7 | EPLE↓LQAG | 135 | | |
| RS34 | MMP-2, 7, 9, 14, | EPLE↓LRAG | 136 | | |
| | matriptase | | | | |
| RS35 | MMP-7 | EPLE↓LKAG | 137 | | |
| RS36 | MMP-2, 7, 9, 14 | EPLE↓LIAG | 138 | | |
| RS37 | Elastase-2 | LGPV↓SGVP | 139 | —/—/—/VIAT/—/—/—/— | |
| RS38 | Granzyme-B | VAGD↓SLEE | 140 | V/—/—/D/—/—/—/— | |
| RS39 | MMP-12 | GPAG↓LGGA | 141 | G/PA/—/G/L/—/G/— | 212 |
| RS40 | MMP-13 | GPAG↓LRGA | 142 | G/P/—/G/L/—/GA/— | 213 |
| RS41 | MMP-17 | APLG↓LRLR | 143 | —/PS/—/—/LQ/—/LT/— | |
| RS42 | MMP-20 | PALP↓LVAQ | 144 | | |
| RS43 | TEV | ENLYFQ↓G | 145 | ENLYFQ/G/S | 214 |
| RS44 | Enterokinase | DDDK↓IVGG | 146 | DDDK/IVGG | 215 |
| RS45 | Protease 3C | LEVLFQ↓GP | 147 | LEVLFQ/GP | 216 |
| | (PreScission ™) | | | | |
| RS46 | Sortase A | LPKT↓GSES | 148 | L/P/KEAD/T/G/—/EKS/S | 217 |
| RS47 | Trypsin | K↓X** or R↓X | | K/X or R/X | |
| RS48 | Trypsin | R↓X** | | SASRSA | 218 |
| RS49 | uPA | SGR↓SA | 149 | S/G/R/SRKA/AGSVR | 219 |
| RS50 | tPA | YGR↓ SA | 150 | RYFLI/GA/R/RVAS/AG | |
| RS51 | PSA | SSYY↓ SG | 151 | S/S/FY/Y/S/G | 220 |
| RS52 | DESC1 | RRAR↓VVGG | 152 | R/RAL/ALY/R/AV/V/G/G | 221 |
| RS53 | Hepsin | RQLR↓VVGG | 153 | R/RQ/YL/R/V/V/G/G | 222 |
| RS54 | Matriptase-2 | RRAR↓VVGG | 154 | R/R/A/R/AV/V/G/G | 223 |
| RS55 | MT-SP1/Matriptase | RQAR↓VVGG | 155 | R/QR/A/R/AVY/V/G/G | 224 |
| RS56 | PSMA | N↓ γ N | | N γ N | |
| RS57 | Cathepsin C | GF↓FY | 156 | GP/FWR/**/— | |
| RS58 | Cathepsin D | F↓IK | | FL/IV/KE | |
| RS59 | Cathepsin E | F↓IK | | FL/IV/KE | |
| RS60 | Cathepsin F | WLR↓ | | WYRNle/L/RKQ | |
| RS61 | Cathepsin K | KPR↓ | | KMGH/ILPNle/RKQ | |
| RS62 | Cathepsin L | KFR↓ | | RKLnL/FYW/RKQ | |
| RS63 | Cathepsin S | RVK↓ | | RPI/VLMnL/RKQ | |
| RS64 | Cathepsin V/L2 | PWR↓ | | PNleR/WYF/RKQ | |
| RS65 | MMP | PLG↓HofOrnL | 157 | | |
| RS66 | MMP | EPCitF↓HofYL | 158 | | |
| RS67 | MMP-2 | PQG↓IAGQ | 159 | | |
| RS68 | MMP-2 | PQG↓IMelG | 160 | | |
| RS69 | MMP-9 | AALG↓NvaP | 161 | | |
| RS70 | MMP-9 | GPQG↓IAGQR | 162 | | |
| RS71 | MMP-9 | SGKIPRT↓ATA | 163 | P/R/PSTRA/Hy/ST | |
| RS72 | MMP-9 | SGPLF↓YSVTA | 164 | | |
| RS73 | MMP-9 | PLR↓LSW | 165 | | |
| RS74 | MMP-9 | GKGPRQ↓ITA | 166 | | |
| RS75 | MMP-9 | SGRR↓LIHHT | 167 | S/G/R/R/L/IL | 225 |
| RS76 | MMP-9 | SGQPHY↓LTTA | 168 | | |
| RS77 | MMP-9 | SG↓LKALM | 169 | | |
| RS78 | MMP-9 | SGFGSRY↓LTA | 170 | | |
| RS79 | MMP-9 | SGLRPAK↓STA | 171 | | |
| RS80 | MMP-9 | LGP↓STST | 172 | | |
| RS81 | MMP-9 | PQG↓VR | 173 | | |
| RS82 | MMP-9 | PSG↓LP | 174 | P/S/G/L/HyP | 226 |
| RS83 | MMP-9 | PAG↓VQ | 175 | | |
| RS84 | MMP-9 | PSG↓RD | 176 | | |
| RS85 | MMP-9 | PPG↓IV | 177 | P/PG/G/Hy/HyR | |
| RS86 | MMP-9 | PEN↓FF | 178 | | |
| RS87 | MMP-9 | PLK↓LM | 179 | | |
| RS88 | MMP-9 | PGA↓YH | 180 | | |
| RS89 | MMP-9 | AIH↓IQ | 181 | | |
| RS90 | MMP-9 | HFF↓KN | 182 | | |
| RS91 | MMP-9 | GLS↓LS | 183 | | |
| RS92 | MMP-9 | ASD↓YK | 184 | | |
| RS93 | MMP-2, MMP-9 | GPLG↓MLSQ | 185 | P/Hy/G/Hy/HyWR | |
| RS94 | MMP-2, MMP-9 | CG↓LDD | 186 | | |
| RS95 | MMP-2, MMP-9, | GPQG↓IWGQ | 187 | | |
| | MT1-MMP | | | | |
| RS96 | MMP-7 | RPLA↓LWRS | 188 | | |
| RS97 | MMP-7 | GPLG↓LARK | 189 | | |
| RS98 | Hk2 | GKAFR↓RL | 190 | | |
| RS99 | MMP-9, uPA | RPSA↓SRSA | 191 | | |
| RS100 | MMP-2 | PLGLDpaAR | 192 | | |
| RS101 | MMP-9 | | | P/LMVQChaHofNva/ | |
| | | | | G/LIYSFC/ST | |
| RS102 | MMP-9 | PChaG↓SmcHA | 193 | P/LCha/G/LSmc/HW/A | |
| RS103 | MMP-13, MMP-8 | PChaGNvaHAdF | 194 | | |
| RS104 | ADAM10 | PTASA↓LKG | 195 | P/T/A/AS/A/LFYQ/KRTI/ | 227 |
| | | | | GAS | |
| RS105 | ADAM17 | PRPAA↓VKGT | 196 | P/HR/P/AS/A/VIL/KRTVI/ | |
| | | | | GST/TP | |
| RS106 | Cathepsin B | V↓Cit | | | |
| RS107 | Cathepsin B | F↓K | | | |
| RS108 | Elastase | AA↓PV | 197 | | |
| RS109 | Cathepsin D | GPIC↓FRLG | 198 | | |
| RS110 | Plasmin | A↓FK | | | |
| RS111 | Legumain | AAN↓L | 199 | | |
| RS112 | Legumain | PTN↓ | | PTAWS/TPASI/N | |
| RS113 | Meprin | | | ED/GTAV/— | |
| RS114 | Meprin A | F↓SPFR | 200 | SFAMTY/ | |
| | | | | SFAMTY/P/PVIGA/— | |
| RS115 | Meprin B | E↓EEAY | 201 | DE/DE/YEFDG/ PVIGA/— | |
| RS116 | Neprilysin | _{β}-AIA↓L | 202 | _{β}-A/LI/A/L | |
| RS117 | ADAMTS4 | E↓VQRKTGT | 203 | E/AFVLMY/(−)/RK/—— (−)/ST | |
| RS118 | ADAMTS4 | DVQE↓FRGVTAVIR | 204 | | |
| RS119 | ADAMTS4 | HNE↓FRQRETYMVF | 205 | | |
| RS120 | ADAMTS5 | KEEE↓GLGS | 206 | | |
| RS121 | ADAMTS5 | GELE↓GRGT | 207 | | |
| RS122 | ADAMTS5 | NITEGE↓ARGS | 208 | | |
| RS123 | ADAMTS5 | TAQE↓AGEG | 209 | | |
| RS124 | ADAMTS5 | VSQE↓LGQR | 210 | | |
| RS125 | ADAMTS5 | PTAQE↓AGE | 211 | | |

↓indicates cleavage site
Special amino acid abbreviation:
Cit: Citrilline;
Cha: β-cyclohexylalanine;
Hof: homophenylalanine;
Nva: aminosuberic acid;
Dpa: D-phenylalanine;
Nle: Norleucine;
Smc: S-methylcysteine
*the listing of multiple amino acids before, between, or after a slash indicate alternative amino acids that can be substituted at the position;
“—” indicates that any amino acid may be substituted for the corresponding amino acid indicated in the middle column
**x is any L-amino acid other than proline
Hy is any hydrophobic L-amino acid
γ indicates that bond is a gamma carboxy linkage

### III). XTEN of the Targeted Conjugate Compositions

The present invention relates, in part, to extended recombinant polypeptides (XTEN) sequences engineered for use in targeted conjugate compositions. Such compositions are useful as fusion partners for the creation of fusion proteins as well as reagent conjugation partners to create targeted conjugate compositions. Additionally, it is an object of the present invention to provide methods to create the compositions.

By way of illustrative example, the XTENs capable of linking or fusing to one or more fusion partners partners for the creation of the subject compositions, which include other XTEN, PCM, targeting moieties or CCD to be conjugated to small molecule payloads, resulting in the targetedconjugate compositions, are specifically engineered to confer certain properties on the resulting compositions, including enhanced solubility, enhanced pharmacokinetic properties, increased mass and hydrodynamic radius to reduce extravasation, as well as a shielding effect to reduce undesireable interaction with otherwise healthy tissues and resultant side effects or toxicity. In some cases, XTEN are designed to incorporate defined numbers of reactive amino acids for linking to the targeting moieties or to permit the creation of multivalent constructs where an XTEN serves as either the backbone to which multiple fusion proteins are attached or to permit conjugation to trivalent or quadravalent linkers via cross-linkers or azide/alkyne reactants. The present invention also provides methods to create such engineered XTEN polymers for use in creating the subject compositions.

In another aspect, the invention provides XTEN polymers comprising defined numbers of cross-linkers or azide/alkyne reactants useful as reactant conjugation partners in the creation of monomeric and multimeric configurations, as well as methods of the preparation of such reactants. The XTEN comprising cross-linkers or azide/alkyne reactants are used as reactants in the conjugation of targeting moieties, other XTEN or other fusion proteins to result in specifically designed conjugate compositions used to achieve the desired physical, pharmaceutical, targeting, and pharmacological properties, including differential toxicity to target tissues.

In another aspect, the invention provides compositions of XTEN including combinations of different fusion proteins or targeting moieties, in defined numbers in either monomeric or multimeric configurations to provide compositions with enhanced targeting, pharmaceutical, pharmacokinetic, and pharmacologic properties, including differential toxicity to diseased target tissues compared to healthy tissues. Such compositions linked to such payloads may have utility, when adminisered to a subject, in the prevention, treatment or amelioration of diseases, with a beneficial response due to the pharmacologic or biologic effect of the payload.

4. XTEN: Extended Recombinant Polypeptides

In one aspect, the invention provides XTEN polypeptide compositions that are useful as fusion partners or as conjugation partners to link to one or more targeting moieties, peptidyl cleavage moieties, CCD, or fusion proteins having the foregoing components, either by recombinant fusion or via a cross-linker reactant that, when combined with the drug or biologic payloads linked to the CCD, result in the targeted conjugate compositions.

In some embodiments, XTEN are polypeptides with non-naturally occurring, substantially non-repetitive sequences having a low degree or no secondary or tertiary structure under physiologic conditions. XTEN typically have from about 36 to about 1000 or more amino acids, of which the majority or the entirety are small hydrophilic amino acids. As used herein, “XTEN” specifically excludes whole antibodies or antibody fragments (e.g. single-chain antibodies and Fc fragments). XTEN polypeptides have utility as fusion and as conjugation partners in that they serve in various roles, conferring certain desirable properties when joined, linked, or fused to a targeting moiety, another XTEN, or other fusion partners. The resulting compositions have enhanced properties, such as enhanced pharmacokinetic, physicochemical, pharmacologic, and improved toxicologic and pharmaceutical properties compared to the corresponding payloads or targeting moieties not linked to XTEN, making them useful in the treatment of certain conditions for which the payloads or targeting moieties are known in the art to be used.

The unstructured characteristic and physicochemical properties of the XTEN result, in part, from the overall amino acid composition that is typically disproportionately limited to 4-6 types of hydrophilic amino acids, the linking of the amino acids in a quantifiable, substantially non-repetitive design, and from the resulting length and/or configuration of the XTEN polypeptide. In an advantageous feature common to XTEN but uncommon to native polypeptides, the properties of XTEN disclosed herein are not tied to absolute primary amino acid sequences, as evidenced by the diversity of the exemplary sequences of Tables 10 and 11 that, within varying ranges of length, possess similar properties and confer enhanced properties on the payloads or targeting moieties to which they are linked, many of which are documented in the Examples. Indeed, it is specifically contemplated that the compositions of the invention not be limited to those XTEN specifically enumerated in Tables 10 and 11, but, rather, the embodiments include sequences having at least 90%, 91%, 92%, 93%, 94%, 95%, 96%, 97%, 98%, or 99% sequence identity to the sequences of Tables 10 and 11 as they exhibit the properties of XTEN described below. It has been established that such XTEN have properties more like non-proteinaceous, hydrophilic polymers (such as polyethylene glycol, or “PEG”) than they do proteins. In some embodiments, the XTEN of the present invention exhibit one or more of the following advantageous properties: defined and uniform length (for a given sequence), conformational flexibility, reduced or lack of secondary structure, high degree of random coil formation, high degree of aqueous solubility, high degree of protease resistance, low immunogenicity, low binding to mammalian receptors, a defined degree of charge, and increased hydrodynamic (or Stokes) radii; properties that are similar to certain hydrophilic polymers (e.g., polyethylene glycol) that make them particularly useful as conjugation partners.

The XTEN component(s) of the subject fusion proteins and conjugates are designed to behave like denatured peptide sequences under physiological conditions, despite the extended length of the polymer. “Denatured” describes the state of a peptide in solution that is characterized by a large conformational freedom of the peptide backbone. Most peptides and proteins adopt a denatured conformation in the presence of high concentrations of denaturants or at elevated temperature. Peptides in denatured conformation have, for example, characteristic circular dichroism (CD) spectra and are characterized by a lack of long-range interactions as determined by NMR. “Denatured conformation” and “unstructured conformation” are used synonymously herein. In some embodiments, the invention provides XTEN sequences that, under physiologic conditions, resemble denatured sequences that are largely devoid of secondary structure. In other cases, the XTEN sequences are substantially devoid of secondary structure under physiologic conditions. “Largely devoid,” as used in this context, means that less than 50% of the XTEN amino acid residues of the XTEN sequence contribute to secondary structure as measured or determined by the means described herein. “Substantially devoid,” as used in this context, means that at least about 60%, or about 70%, or about 80%, or about 90%, or about 95%, or about 97%, or at least about 99% of the XTEN amino acid residues of the XTEN sequence do not contribute to secondary structure, as measured or determined by the methods described herein, including algorithms or spectrophotometric assays.

A variety of well-established methods and assays are known in the art for determining and confirming the physicochemical properties of the subject XTEN. Such properties include but are not limited to secondary or tertiary structure, solubility, protein aggregation, stability, absolute and apparent molecular weight, purity and uniformity, melting properties, contamination and water content. The methods to measure such properties include analytical centrifugation, EPR, HPLC-ion exchange, HPLC-size exclusion chromatography (SEC), HPLC-reverse phase, light scattering, capillary electrophoresis, circular dichroism, differential scanning calorimetry, fluorescence, HPLC-ion exchange, HPLC-size exclusion, IR, NMR, Raman spectroscopy, refractometry, and UV/Visible spectroscopy. In particular, secondary structure can be measured spectrophotometrically, e.g., by circular dichroism spectroscopy in the “far-UV” spectral region (190-250 nm). Secondary structure elements, such as alpha-helix and beta-sheet, each give rise to a characteristic shape and magnitude of CD spectra, as does the lack of these structure elements, and an exemplary CD assay of an XTEN is provided in the Examples and supports the conclusion that XTEN lack secondary structure. Secondary structure can also be predicted for a polypeptide sequence via certain computer programs or algorithms, such as the well-known Chou-Fasman algorithm (Chou, P. Y., et al. (1974) Biochemistry, 13: 222-45) and the Garnier-Osguthorpe-Robson algorithm (“Gor algorithm”) (Gamier J, Gibrat J F, Robson B. (1996), GOR method for predicting protein secondary structure from amino acid sequence. Methods Enzymol 266:540-553), as described in US Patent Application Publication No. 20030228309A1. For a given sequence, the algorithms can predict whether there exists some or no secondary structure at all, expressed as the total and/or percentage of residues of the sequence that form, for example, alpha-helices or beta-sheets or the percentage of residues of the sequence predicted to result in random coil formation (which lacks secondary structure). Polypeptide sequences can be analyzed using the Chou-Fasman algorithm using sites on the world wide web at, for example, fasta.bioch.virginia.edu/fasta_www2/fasta_www.cgi?rm=miscl and the Gor algorithm at npsa-pbil.ibcp.fr/cgi-bin/npsa_automat.pl?page=npsa_gor4.html (both accessed onOct. 30, 2015). Random coil can be determined by a variety of methods, including by using intrinsic viscosity measurements, which scale with chain length in a conformation-dependent way (Tanford, C., Kawahara, K. & Lapanje, S. (1966) J. Biol. Chem. 241, 1921-1923), as well as by size-exclusion chromatography (Squire, P. G., Calculation of hydrodynamic parameters of random coil polymers from size exclusion chromotography and comparison with parameters by conventional methods. Journal of Chromatography, 1981, 5,433-442). Additional methods are disclosed in Arnau, et al., Prot Expr and Purif (2006) 48, 1-13.

In one embodiment, the XTEN sequences used in the subject conjugates have an alpha-helix percentage ranging from 0% to less than about 5% as determined by the Chou-Fasman algorithm. In another embodiment, the XTEN sequences have a beta-sheet percentage ranging from 0% to less than about 5% as determined by the Chou-Fasman algorithm. In one embodiment, the XTEN sequences of the conjugates have an alpha-helix percentage ranging from 0% to less than about 5% and a beta-sheet percentage ranging from 0% to less than about 5% as determined by the Chou-Fasman algorithm. In one embodiment, the XTEN sequences of the conjugates have an alpha-helix percentage less than about 2% and a beta-sheet percentage less than about 2%. The XTEN sequences of the compositions have a high degree of random coil formation, as determined by the GOR algorithm. In some embodiments, an XTEN sequence has at least about 80%, more preferably at least about 90%, more preferably at least about 91%, more preferably at least about 92%, more preferably at least about 93%, more preferably at least about 94%, more preferably at least about 95%, more preferably at least about 96%, more preferably at least about 97%, more preferably at least about 98%, and most preferably at least about 99% random coil formation, as determined by the GOR algorithm. In one embodiment, the XTEN sequences of the targeted conjugate compositions have an alpha-helix percentage ranging from 0% to less than about 5% and a beta-sheet percentage ranging from 0% to less than about 5% as determined by the Chou-Fasman algorithm and at least about 90% random coil formation as determined by the GOR algorithm. In another embodiment, the XTEN sequences of the disclosed compositions have an alpha-helix percentage less than about 2% and a beta-sheet percentage less than about 2% as determined by the Chou-Fasman algorithm and at least about 90% random coil formation as determined by the GOR algorithm. In another embodiment, the XTEN sequenes of the compositions are substantially lacking secondary structure as measured by circular dichroism.

The selection criteria for the XTEN to be linked to the components used to create the targeted conjugate compositions generally relate to attributes of physicochemical properties and conformational structure of the XTEN that is, in turn, used to confer enhanced pharmaceutical, pharmacologic, and pharmacokinetic properties to the compositions.

### 1. Non-Repetitive Sequences

It is specifically contemplated that the subject XTEN sequences included in the subject conjugate composition embodiments are substantially non-repetitive. In general, repetitive amino acid sequences have a tendency to aggregate or form higher order structures, as exemplified by natural repetitive sequences such as collagens and leucine zippers. These repetitive amino acids may also tend to form contacts resulting in crystalline or pseudocrystaline structures. In contrast, the low tendency of non-repetitive sequences to aggregate enables the design of long-sequence XTENs with a relatively low frequency of charged amino acids that would otherwise be likely to aggregate if the sequences were repetitive. The non-repetitiveness of a subject XTEN can be observed by assessing one or more of the following features. In one embodiment, a substantially non-repetitive XTEN sequence has no three contiguous amino acids in the sequence that are identical amino acid types unless the amino acid is serine, in which case no more than three contiguous amino acids are serine residues. In another embodiment, as described more fully below, the invention provides a substantially non-repetitive XTEN sequence in which 80-99% of the sequence is comprised of motifs of 12 amino acid residues wherein the motifs consist of 4, 5 or 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In another embodiment, the invention provides a substantially non-repetitive XTEN sequence in which at least about 90% of the sequence consists of motifs of 12 amino acid residues wherein the motifs consist of 4, 5 or 6 types of amino acids selected from glycine (G), alanine (A), serine (S), threonine (T), glutamate (E) and proline (P), and wherein the sequence of any two contiguous amino acid residues in any one motif is not repeated more than twice in the sequence motif. In another embodiment, the invention provides a substantially non-repetitive XTEN sequence in which at least about 90% of the sequence consists of motifs of 12 amino acid residues selected from the group consisting of the sequences set forth in Table 9. In another embodiment, the invention provides a substantially non-repetitive XTEN sequence in which at least 90%, at least 91%, at least 92%, at least 93%, at least 94%, at least 95%, at least 96%, at least 97%, at least 98%, at least 98%, or 100% of the sequence consists of motifs of 12 amino acid residues selected from the group consisting of the AE sequences set forth in Table 9. In another embodiment, the invention provides a substantially non-repetitive XTEN sequence in which at least 90%, at least 91%, at least 92%, at least 93%, at least 94%, at least 95%, at least 96%, at least 97%, at least 98%, at least 98%, or 100% of the sequence consists of motifs of 12 amino acid residues selected from the group consisting of the AF sequences set forth in Table 9. In another embodiment, the invention provides a substantially non-repetitive XTEN sequence in which at least 90%, at least 91%, at least 92%, at least 93%, at least 94%, at least 95%, at least 96%, at least 97%, at least 98%, at least 98%, or 100% of the sequence consists of motifs of 12 amino acid residues selected from the group consisting of the AG sequences set forth in Table 9.

The degree of repetitiveness of a polypeptide or a gene can be measured by computer programs or algorithms or by other means known in the art. According to the current invention, algorithms to be used in calculating the degree of repetitiveness of a particular polypeptide, such as an XTEN, are disclosed herein, and examples of sequences analyzed by algorithms are provided (see Examples, below). In one embodiment, the repetitiveness of a polypeptide of a predetermined length can be calculated (hereinafter “subsequence score”) according to the formula given by Equation I:

$$Subsequence score = \frac{{\sum }_{i = 1}^{m} {Count}_{i}}{m} \qquad I$$

wherein: m=(amino acid length of polypeptide)−(amino acid length of subsequence)+1; and Count,=cumulative number of occurrences of each unique subsequence within

sequence,

An algorithm termed “SegScore” was developed to apply the foregoing equation to quantitate repetitiveness of polypeptides, such as an XTEN, providing the subsequence score wherein sequences of a predetermined amino acid length “n” are analyzed for repetitiveness by determining the number of times (a “count”) a unique subsequence of length “s” appears in the set length, divided by the absolute number of subsequences within the predetermined length of the sequence. The subsequence score of any given polypeptide will depend on the absolute number of unique subsequences and how frequently each unique subsequence (meaning a different amino acid sequence) appears in the predetermined length of the sequence.

In the context of the present invention, “subsequence score” means the sum of occurrences of each unique 3-mer frame across 200 consecutive amino acids of the XTEN polypeptide divided by the absolute number of unique 3-mer subsequences within the 200 amino acid sequence. Examples of such subsequence scores derived from 200 consecutive amino acids of repetitive and non-repetitive polypeptides are presented in Example 32. In one embodiment, the invention provides a XTEN-conjugate comprising one XTEN in which the XTEN has a subsequence score less than 12, more preferably less than 10, more preferably less than 9, more preferably less than 8, more preferably less than 7, more preferably less than 6, and most preferably less than 5. In another embodiment, the invention provides targeted conjugate compositions comprising at least two XTEN in which each individual XTEN has a subsequence score of less than 10, or less than 9, or less than 8, or less than 7, or less than 6, or less than 5, or less. In yet another embodiment, the invention provides XTEN compositions comprising at least three linked XTEN in which each individual XTEN has a subsequence score of less than 10, or less than 9, or less than 8, or less than 7, or less than 6, or less than 5, or less. In the embodiments of the XTEN compositions described herein, an XTEN with a subsequence score of 10 or less (i.e., 9, 8, 7, etc.) is characterized as substantially non-repetitive.

In another embodiment, the average repetitiveness of a polypeptide of any length can be calculated (hereinafter “average subsequence score”) according to the formula given by Equation II:

$$Average subsequence score = \frac{{\sum }_{i = 1}^{n} ( \frac{{Count}_{i}}{m} )}{n} \qquad II$$

wherein: n=(amino acid length of polypeptide)−(amino acid length of block)+1;

m=(amino acid length of block)−(amino acid length of subsequence)+1; and

Count,=cumulative number of occurrences of each unique subsequence within block,

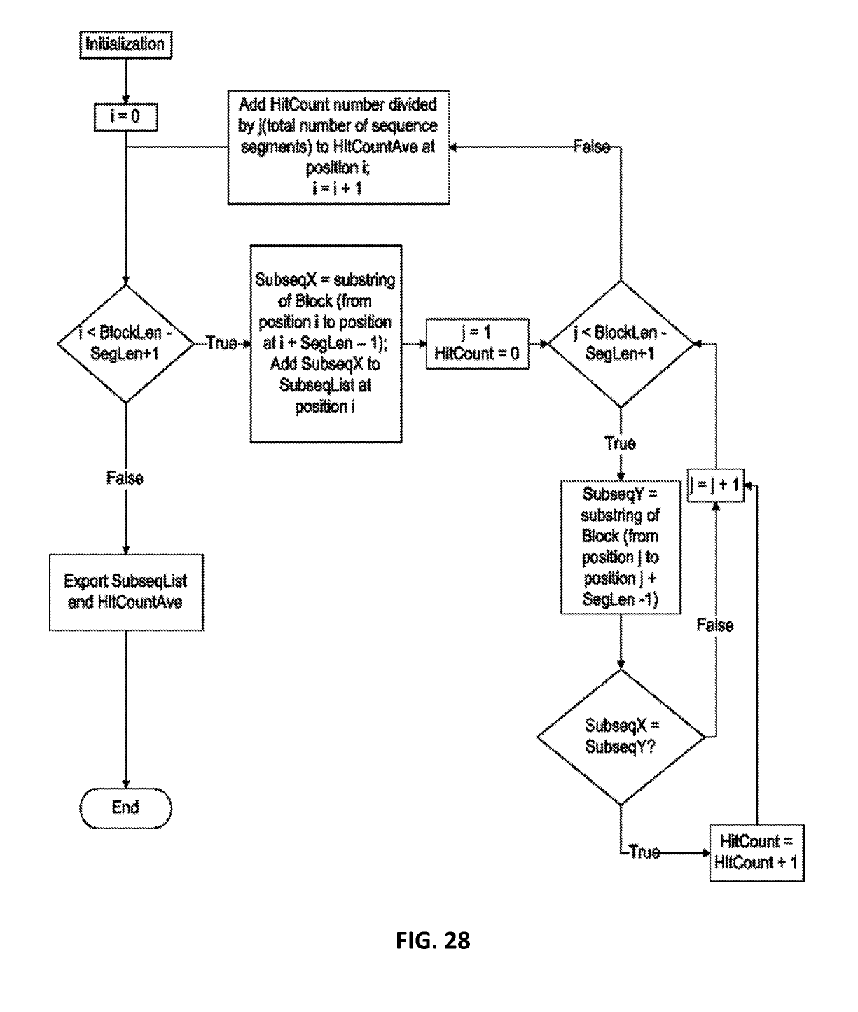

A second algorithm termed “BlockScore” was developed to implement the foregoing equation to quantitate the average repetitiveness of a polypeptide, such as an XTEN, so that the repetitiveness of polypeptides of different lengths could be compared. FIG. 28 depicts a logic flowchart of the BlockScore algorithm. For BlockScore (or algorithms of similar design or purpose), the subject polypeptide sequence can treated as a series of overlapping segments of equal length that are shorter than the length of the polypeptide (hereinafter, “blocks”). In turn, each block can be treated as a series of overlapping segments of equal length that are shorter than the length of the block (hereinafter, “subsequence”). The BlockScore algorithm determines a score, (hereinafter, “average subsequence score”) by first applying the SegScore algorithm to each of the individual overlapping blocks in a polypeptide to create an array of subsequence scores and then determining the average of the subsequence scores for all of the blocks of the polypeptide. For example, a polypeptide of 200 amino acid residues length has a total of 165 overlapping 36-amino acid “blocks” and 198 3-mer amino acid “subsequences”, but the number of unique 3-mer subsequences (meaning a unique specific sequence of three amino acids) found within each block will depend on the amount of repetitiveness within the block; a polypeptide with blocks with a high degree of repetitiveness will generally have fewer unique 3-mer subsequences. The average subsequence score that is generated by BlockScore or by application of the foregoing Equation II to a polypeptide is reflective of the degree of repetitiveness and the values assigned to two variables, 1) the length of the block in amino acid residues, and 2) the length of the subsequence in amino acid residues. The invention contemplates that the variable “subsequence” can be a peptide length of 3 to about 10 amino acid residues and that the variable “block” can be a peptide length of about 20 to about 800 amino acid residues. In a preferred method, and as applied (except as where noted otherwise) to the embodiments that follow, “average subsequence score” for a polypeptide is determined by application of the foregoing Equation II or the BlockScore algorithm to a polypeptide sequence wherein the block length is set at 36 amino acids and the subsequence length is set at 3 amino acids.

In some embodiments, the present invention provides targeted conjugate compositions comprising one or more XTEN in which each XTEN has a average subsequence score of 3 or less, and more preferably less than 2. In another embodiment, the invention provides targeted conjugate compositions comprising two XTEN in which at least one XTEN has a average subsequence score of 3 or less, and more preferably less than 2. In yet another embodiment, the invention provides targeted conjugate compositions comprising at least three XTEN in which each individual XTEN has an average subsequence score of 3 or less, and more preferably less than 2. In the embodiments of the targeted conjugate compositions described herein, an XTEN component of a composition with an average subsequence score of 3 or less is “substantially non-repetitive.”

It has been established that the non-repetitive characteristic of XTEN of the present invention together with the particular types of amino acids that predominate in the XTEN, rather than the absolute primary sequence, confers one or more of the enhanced physicochemical and biological properties of the XTEN and the resulting targeted conjugate composition. Accordingly, while the sequences of Tables 10 and 11 are exemplary, they are not intended to be limiting, as sequences having at least 90%, 91%, 92%, 93%, 94%, 95%, 96%, 97%, 98%, or at least 99% sequence identity to the sequences of Tables 10 and 11 exhibit the enhanced properties of XTEN. These enhanced properties include a high degree of expression of the XTEN protein in the host cell, greater genetic stability of the gene encoding XTEN, and confer a greater degree of solubility, less tendency to aggregate, and enhanced pharmacokinetics of the resulting targeted conjugate compared to payloads or proteins having repetitive sequences not conjugated to XTEN. These enhanced properties permit more efficient manufacturing, greater uniformity of the final product, lower cost of goods, and/or facilitate the formulation of pharmaceutical preparations of the subject compositions containing extremely high protein concentrations, in some cases exceeding 100 mg/ml. Additionally, the XTEN polypeptide sequences of the conjugates are designed to have a low degree of internal repetitiveness in order to reduce or substantially eliminate immunogenicity when administered to a mammal. Polypeptide sequences composed of short, repeated motifs largely limited to only three amino acids, such as glycine, serine and glutamate, may result in relatively high antibody titers when administered to a mammal despite the absence of predicted T-cell epitopes in these sequences. This may be caused by the repetitive nature of polypeptides, as it has been shown that immunogens with repeated epitopes, including protein aggregates, cross-linked immunogens, and repetitive carbohydrates are highly immunogenic and can, for example, result in the cross-linking of B-cell receptors causing B-cell activation. (Johansson, J., et al. (2007) Vaccine, 25 :1676-82; Yankai, Z., et al. (2006) Biochem Biophys Res Commun, 345 :1365-71; Hsu, C. T., et al. (2000) Cancer Res, 60:3701-5); Bachmann MF, et al. Eur J Immunol. (1995) 25(12):3445-3451).

### 2. Exemplary Sequence Motifs and XTEN Segments

The present invention encompasses XTEN used as fusion and conjugation partners that comprise multiple units of shorter sequences, or motifs, in which the amino acid sequences of the motifs are substantially non-repetitive. The non-repetitive property can be met even using a “building block” approach using a small library of sequence motifs that are multimerized to create the XTEN sequences. While an XTEN sequence may consist of multiple units of as few as four different types of sequence motifs, because the motifs themselves generally consist of non-repetitive amino acid sequences, the overall XTEN sequence is designed to render the sequence substantially non-repetitive.

It is specifically intended the range of XTEN lengths for use in the subject compositions of the disclosure are not limiting and that the XTEN can comprise any number of amino acid residues from 36 to 1500 or more and be encompassed by the embodiments of the invention.

In one embodiment, XTEN comprises a sequence in which at least 90%, or 91%, or 92%, or 93%, or 94%, or 95%, or 96%, or 97%, or 98%, or at least 99% of the amino acid residues are four to six types of amino acids selected from the group consisting of glycine (G), alanine (A), serine (S), threonine (T), glutamate (E) and proline (P) that are arranged in a substantially non-repetitive sequence. In one embodiment, an XTEN sequence is made of 4, 5, or 6 types of amino acids selected from the group consisting of glycine (G), alanine (A), serine (S), threonine (T), glutamate (E) and proline (P). In other embodiments, at least about 90%, or 91%, or 92%, or 93%, or 94%, or 95%, or 96%, or 97%, or 98%, or at least 99% of the XTEN sequence consists of non-overlapping sequence motifs wherein each of the motifs has 12 amino acid residues consisting of 4 to 6 types of amino acids selected from glycine (G), alanine (A), serine (S), threonine (T), glutamate (E) and proline (P), and wherein the content of any one amino acid type in the full-length XTEN does not exceed 40%, or 30%, or about 25%, or about 17%, or about 12%, or about 8%. In yet other embodiments, at least about 90%, or about 91%, or about 92%, or about 93%, or about 94%, or about 95%, or about 96%, or about 97%, or about 98%, or about 99%, to about 100% of the XTEN sequence consists of non-overlapping sequence motifs wherein each of the motifs has 12 amino acid residues consisting of glycine (G), alanine (A), serine (S), threonine (T), glutamate (E) and proline (P).

In some embodiments, the invention provides targeted conjugate compositions comprising one, or two, or three, or four substantially non-repetitive XTEN sequence(s) of at least about 100 to about 1200 amino acid residues each, or cumulatively about 200 to about 2000 amino acid residues wherein at least about 90%, or about 91%, or about 92%, or about 93%, or about 94%, or about 95%, or about 96%, or about 97%, or about 98%, or about 99% to about 100% of the sequence consists of multiple units of four or more non-overlapping sequence motifs selected from the amino acid sequences of Table 9. In the embodiments hereinabove described in this paragraph, the motifs or portions of the motifs incorporated into the XTEN can be selected and assembled using the methods described herein to achieve an XTEN of at least 36, at least 42, at least 72, at least 144, at least 288, at least 576, at least 864, at least 1000, at least 1500 amino acid residues, or any intermediate length. Non-limiting examples of XTEN sequences useful for incorporation into the XTEN of the subject compositions are presented in Tables 10 and 11. It is intended that a specified sequence mentioned relative to Table 10 or Table 11 has that sequence set forth in the respective table, while a generalized reference to an AE144 sequence, for example, is intended to encompass any AE sequence having 144 amino acid residues, or a generalized reference to an AG864 sequence, for example, is intended to encompass any AG sequence having 864 amino acid residues, etc.

TABLE 9
XTEN Sequence Motifs of 12 Amino Acids
and Motif Families

| Motif | | SEQ |
|---|---|---|
| Family* | Motif Sequence | ID NO: |
| AE, | GSPAGSPTSTEE | 228 |
| AE | GSEPATSGSETP | 229 |
| AE | GTSESATPESGP | 230 |
| AE | GTSTEPSEGSAP | 231 |
| AF | GSTSESPSGTAP | 232 |
| AF | GTSTPESGSASP | 233 |
| AF | GTSPSGESSTAP | 234 |
| AF | GSTSSTAESPGP | 235 |
| AG | GTPGSGTASSSP | 236 |
| AG | GSSTPSGATGSP | 237 |
| AG | GSSPSASTGTGP | 238 |
| AG | GASPGTSSTGSP | 239 |

*Denotes individual motif sequences that, when used together in various permutations, results in a “family sequence”

TABLE 10
XTEN Polypeptides

| XTEN | | SEQ ID |
|---|---|---|
| Name | Amino Acid Sequence | NO: |
| AE144 | GSEPATSGSETPGTSESATPESGPGSEPATSGSETPGSPAGSPTSTEEGTST | 240 |
| | EPSEGSAPGSEPATSGSETPGSEPATSGSETPGSEPATSGSETPGTSTEPSE | |
| | GSAPGTSESATPESGPGSEPATSGSETPGTSTEPSEGSAP | |
| AE144_1A | SPAGSPTSTEEGTSESATPESGPGTSTEPSEGSAPGSPAGSPTSTEEGTSTE | 241 |
| | PSEGSAPGTSTEPSEGSAPGTSESATPESGPGSEPATSGSETPGSEPATSGS | |
| | ETPGSPAGSPTSTEEGTSESATPESGPGTSTEPSEGSAPG | |
| AE144_2A | TSTEPSEGSAPGSPAGSPTSTEEGTSTEPSEGSAPGTSTEPSEGSAPGTSES | 242 |
| | ATPESGPGTSTEPSEGSAPGTSESATPESGPGSEPATSGSETPGTSTEPSEG | |
| | SAPGTSTEPSEGSAPGTSESATPESGPGTSESATPESGPG | |
| AE144_2B | TSTEPSEGSAPGSPAGSPTSTEEGTSTEPSEGSAPGTSTEPSEGSAPGTSES | 243 |
| | ATPESGPGTSTEPSEGSAPGTSESATPESGPGSEPATSGSETPGTSTEPSEG | |
| | SAPGTSTEPSEGSAPGTSESATPESGPGTSESATPESGPG | |
| AE144_3A | SPAGSPTSTEEGTSESATPESGPGSEPATSGSETPGTSESATPESGPGTSTE | 244 |
| | PSEGSAPGTSTEPSEGSAPGTSTEPSEGSAPGTSTEPSEGSAPGTSTEPSEG | |
| | SAPGTSTEPSEGSAPGSPAGSPTSTEEGTSTEPSEGSAPG | |
| AE144_3B | SPAGSPTSTEEGTSESATPESGPGSEPATSGSETPGTSESATPESGPGTSTE | 245 |
| | PSEGSAPGTSTEPSEGSAPGTSTEPSEGSAPGTSTEPSEGSAPGTSTEPSEG | |
| | SAPGTSTEPSEGSAPGSPAGSPTSTEEGTSTEPSEGSAPG | |
| AE144_4A | TSESATPESGPGSEPATSGSETPGTSESATPESGPGSEPATSGSETPGTSES | 246 |
| | ATPESGPGTSTEPSEGSAPGTSESATPESGPGSPAGSPTSTEEGSPAGSPTS | |
| | TEEGSPAGSPTSTEEGTSESATPESGPGTSTEPSEGSAPG | |
| AE144_4B | TSESATPESGPGSEPATSGSETPGTSESATPESGPGSEPATSGSETPGTSES | 247 |
| | ATPESGPGTSTEPSEGSAPGTSESATPESGPGSPAGSPTSTEEGSPAGSPTS | |
| | TEEGSPAGSPTSTEEGTSESATPESGPGTSTEPSEGSAPG | |
| AE144_5A | TSESATPESGPGSEPATSGSETPGTSESATPESGPGSEPATSGSETPGTSES | 248 |
| | ATPESGPGTSTEPSEGSAPGSPAGSPTSTEEGTSESATPESGPGSEPATSG | |
| | SETPGTSESATPESGPGSPAGSPTSTEEGSPAGSPTSTEEG | |
| AE144_6B | TSTEPSEGSAPGTSESATPESGPGTSESATPESGPGTSESATPESGPGSEPA | 249 |
| | TSGSETPGSEPATSGSETPGSPAGSPTSTEEGTSTEPSEGSAPGTSTEPSEG | |
| | SAPGSEPATSGSETPGTSESATPESGPGTSTEPSEGSAPG | |
| AF144 | GTSTPESGSASPGTSPSGESSTAPGTSPSGESSTAPGSTSSTAESPGPGSTS | 250 |
| | ESPSGTAPGSTSSTAESPGPGTSPSGESSTAPGTSTPESGSASPGSTSSTAE | |
| | SPGPGTSPSGESSTAPGTSPSGESSTAPGTSPSGESSTAP | |
| AG144_1 | SGTASSSPGSSTPSGATGSPGTPGSGTASSSPGSSTPSGATGSPGSSTPSG | 251 |
| | ATGSPGSSPSASTGTGPGSSPSASTGTGPGASPGTSSTGSPGTPGSGTASS | |
| | SPGSSTPSGATGSPGSSPSASTGTGPGSSPSASTGTGPGASP | |
| AG144_2 | PGSSPSASTGTGPGSSPSASTGTGPGTPGSGTASSSPGSSTPSGATGSPGS | 252 |
| | SPSASTGTGPGASPGTSSTGSPGTPGSGTASSSPGSSTPSGATGSPGTPGS | |
| | GTASSSPGASPGTSSTGSPGASPGTSSTGSPGTPGSGTASSS | |
| AG144_A | GASPGTSSTGSPGSSPSASTGTGPGSSPSASTGTGPGTPGSGTASSSPGSS | 253 |
| | TPSGATGSPGSSPSASTGTGPGASPGTSSTGSPGTPGSGTASSSPGSSTPS | |
| | GATGSPGTPGSGTASSSPGASPGTSSTGSPGASPGTSSTGSP | |
| AG144_B | GTPGSGTASSSPGSSTPSGATGSPGASPGTSSTGSPGTPGSGTASSSPGSS | 254 |
| | TPSGATGSPGSSPSASTGTGPGSSPSASTGTGPGSSTPSGATGSPGSSTPS | |
| | GATGSPGASPGTSSTGSPGASPGTSSTGSPGASPGTSSTGSP | |
| AG144_C | GTPGSGTASSSPGASPGTSSTGSPGASPGTSSTGSPGASPGTSSTGSPGSS | 255 |
| | PSASTGTGPGTPGSGTASSSPGASPGTSSTGSPGASPGTSSTGSPGASPGT | |
| | SSTGSPGSSTPSGATGSPGSSTPSGATGSPGASPGTSSTGSP | |
| AG144_F | GSSPSASTGTGPGSSPSASTGTGPGASPGTSSTGSPGASPGTSSTGSPGSS | 256 |
| | TPSGATGSPGSSPSASTGTGPGASPGTSSTGSPGSSPSASTGTGPGTPGSG | |
| | TASSSPGSSTPSGATGSPGSSTPSGATGSPGASPGTSSTGSP | |
| AG144_3 | GTPGSGTASSSPGSSTPSGATGSPGSSTPSGATGSPGSSPSASTGTGPGSS | 257 |
| | PSASTGTGPGASPGTSSTGSPGTPGSGTASSSPGSSTPSGATGSPGSSPSA | |
| | STGTGPGSSPSASTGTGPGASPGTSSTGSPGASPGTSSTGSP | |
| AG144_4 | GTPGSGTASSSPGSSTPSGATGSPGSSPSASTGTGPGSSPSASTGTGPGAS | 258 |
| | PGTSSTGSPGASPGTSSTGSPGSSTPSGATGSPGSSPSASTGTGPGASPGT | |
| | SSTGSPGSSPSASTGTGPGTPGSGTASSSPGSSTPSGATGSP | |
| AE288_1 | GTSESATPESGPGSEPATSGSETPGTSESATPESGPGSEPATSGSETPGTSE | 259 |
| | SATPESGPGTSTEPSEGSAPGSPAGSPTSTEEGTSESATPESGPGSEPATS | |
| | GSETPGTSESATPESGPGSPAGSPTSTEEGSPAGSPTSTEEGTSTEPSEGSA | |
| | PGTSESATPESGPGTSESATPESGPGTSESATPESGPGSEPATSGSETPGSE | |
| | PATSGSETPGSPAGSPTSTEEGTSTEPSEGSAPGTSTEPSEGSAPGSEPATS | |
| | GSETPGTSESATPESGPGTSTEPSEGSAP | |
| AE288_2 | GSPAGSPTSTEEGTSESATPESGPGSEPATSGSETPGTSESATPESGPGTST | 260 |
| | EPSEGSAPGTSTEPSEGSAPGTSTEPSEGSAPGTSTEPSEGSAPGTSTEPSE | |
| | GSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTS | |
| | TEPSEGSAPGTSESATPESGPGSPAGSPTSTEEGSPAGSPTSTEEGSPAGSP | |
| | TSTEEGTSESATPESGPGTSTEPSEGSAP | |
| AG288_1 | PGASPGTSSTGSPGASPGTSSTGSPGTPGSGTASSSPGSSTPSGATGSPGT | 261 |
| | PGSGTASSSPGSSTPSGATGSPGTPGSGTASSSPGSSTPSGATGSPGSSTPS | |
| | GATGSPGSSPSASTGTGPGSSPSASTGTGPGASPGTSSTGSPGTPGSGTAS | |
| | SSPGSSTPSGATGSPGSSPSASTGTGPGSSPSASTGTGPGASPGTSSTGSP | |
| | GASPGTSSTGSPGSSTPSGATGSPGSSPSASTGTGPGASPGTSSTGSPGSS | |
| | PSASTGTGPGTPGSGTASSSPGSSTPSGATGS | |
| AG288_2 | GSSPSASTGTGPGSSPSASTGTGPGTPGSGTASSSPGSSTPSGATGSPGSS | 262 |
| | PSASTGTGPGASPGTSSTGSPGTPGSGTASSSPGSSTPSGATGSPGTPGSG | |
| | TASSSPGASPGTSSTGSPGASPGTSSTGSPGTPGSGTASSSPGSSTPSGAT | |
| | GSPGASPGTSSTGSPGTPGSGTASSSPGSSTPSGATGSPGSSPSASTGTGP | |
| | GSSPSASTGTGPGSSTPSGATGSPGSSTPSGATGSPGASPGTSSTGSPGAS | |
| | PGTSSTGSPGASPGTSSTGSPGTPGSGTASSSP | |
| AF540 | GSTSSTAESPGPGSTSSTAESPGPGSTSESPSGTAPGSTSSTAESPGPGSTS | 263 |
| | STAESPGPGTSTPESGSASPGSTSESPSGTAPGTSPSGESSTAPGSTSESPS | |
| | GTAPGSTSESPSGTAPGTSPSGESSTAPGSTSESPSGTAPGSTSESPSGTAP | |
| | GTSPSGESSTAPGSTSESPSGTAPGSTSESPSGTAPGSTSESPSGTAPGTST | |
| | PESGSASPGSTSESPSGTAPGTSTPESGSASPGSTSSTAESPGPGSTSSTAE | |
| | SPGPGTSTPESGSASPGTSTPESGSASPGSTSESPSGTAPGTSTPESGSASP | |
| | GTSTPESGSASPGSTSESPSGTAPGSTSESPSGTAPGSTSESPSGTAPGSTS | |
| | STAESPGPGTSTPESGSASPGTSTPESGSASPGSTSESPSGTAPGSTSESPS | |
| | GTAPGTSTPESGSASPGSTSESPSGTAPGSTSESPSGTAPGTSTPESGSASP | |
| | GTSPSGESSTAPGSTSSTAESPGPGTSPSGESSTAPGSTSSTAESPGPGTST | |
| | PESGSASPGSTSESPSGTAP | |
| AE576 | GSPAGSPTSTEEGTSESATPESGPGTSTEPSEGSAPGSPAGSPTSTEEGTST | 264 |
| | EPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAP | |
| AF576 | GSTSSTAESPGPGSTSSTAESPGPGSTSESPSGTAPGSTSSTAESPGPGSTS | 265 |
| | STAESPGPGTSTPESGSASPGSTSESPSGTAPGTSPSGESSTAPGSTSESPS | |
| | GTAPGSTSESPSGTAPGTSPSGESSTAPGSTSESPSGTAPGSTSESPSGTAP | |
| | GTSPSGESSTAPGSTSESPSGTAPGSTSESPSGTAPGSTSESPSGTAPGTST | |
| | PESGSASPGSTSESPSGTAPGTSTPESGSASPGSTSSTAESPGPGSTSSTAE | |
| | SPGPGTSTPESGSASPGTSTPESGSASPGSTSESPSGTAPGTSTPESGSASP | |
| | GTSTPESGSASPGSTSESPSGTAPGSTSESPSGTAPGSTSESPSGTAPGSTS | |
| | STAESPGPGTSTPESGSASPGTSTPESGSASPGSTSESPSGTAPGSTSESPS | |
| | GTAPGTSTPESGSASPGSTSESPSGTAPGSTSESPSGTAPGTSTPESGSASP | |
| | GTSPSGESSTAPGSTSSTAESPGPGTSPSGESSTAPGSTSSTAESPGPGTST | |
| | PESGSASPGSTSESPSGTAPGSTSSTAESPGPGTSTPESGSASPGTSTPESG | |
| | SASP | |
| AGS 76 | PGTPGSGTASSSPGSSTPSGATGSPGSSPSASTGTGPGSSPSASTGTGPGS | 266 |
| | STPSGATGSPGSSTPSGATGSPGASPGTSSTGSPGASPGTSSTGSPGASPG | |
| | TSSTGSPGTPGSGTASSSPGASPGTSSTGSPGASPGTSSTGSPGASPGTSS | |
| | TGSPGSSPSASTGTGPGTPGSGTASSSPGASPGTSSTGSPGASPGTSSTGS | |
| | PGASPGTSSTGSPGSSTPSGATGSPGSSTPSGATGSPGASPGTSSTGSPGT | |
| | PGSGTASSSPGSSTPSGATGSPGSSTPSGATGSPGSSTPSGATGSPGSSPS | |
| | ASTGTGPGASPGTSSTGSPGASPGTSSTGSPGTPGSGTASSSPGASPGTSS | |
| | TGSPGASPGTSSTGSPGASPGTSSTGSPGASPGTSSTGSPGTPGSGTASSS | |
| | PGSSTPSGATGSPGTPGSGTASSSPGSSTPSGATGSPGTPGSGTASSSPGS | |
| | STPSGATGSPGSSTPSGATGSPGSSPSASTGTGPGSSPSASTGTGPGASPG | |
| | TSSTGSPGTPGSGTASSSPGSSTPSGATGSPGSSPSASTGTGPGSSPSASTG | |
| | TGPGASPGTSSTGS | |
| AE624 | MAEPAGSPTSTEEGTPGSGTASSSPGSSTPSGATGSPGASPGTSSTGSPGS | 267 |
| | PAGSPTSTEEGTSESATPESGPGTSTEPSEGSAPGSPAGSPTSTEEGTSTEP | |
| | SEGSAPGTSTEPSEGSAPGTSESATPESGPGSEPATSGSETPGSEPATSGS | |
| | ETPGSPAGSPTSTEEGTSESATPESGPGTSTEPSEGSAPGTSTEPSEGSAPG | |
| | SPAGSPTSTEEGTSTEPSEGSAPGTSTEPSEGSAPGTSESATPESGPGTSTE | |
| | PSEGSAPGTSESATPESGPGSEPATSGSETPGTSTEPSEGSAPGTSTEPSEG | |
| | SAPGTSESATPESGPGTSESATPESGPGSPAGSPTSTEEGTSESATPESGP | |
| | GSEPATSGSETPGTSESATPESGPGTSTEPSEGSAPGTSTEPSEGSAPGTST | |
| | EPSEGSAPGTSTEPSEGSAPGTSTEPSEGSAPGTSTEPSEGSAPGSPAGSPT | |
| | STEEGTSTEPSEGSAPGTSESATPESGPGSEPATSGSETPGTSESATPESGP | |
| | GSEPATSGSETPGTSESATPESGPGTSTEPSEGSAPGTSESATPESGPGSP | |
| | AGSPTSTEEGSPAGSPTSTEEGSPAGSPTSTEEGTSESATPESGPGTSTEPS | |
| | EGSAP | |
| AE864 | GSPAGSPTSTEEGTSESATPESGPGTSTEPSEGSAPGSPAGSPTSTEEGTST | 268 |
| | EPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPGTSTEPSEGSA | |
| | PGSEPATSGSETPGTSESATPESGPGTSTEPSEGSAP | |
| AF864 | GSTSSTAESPGPGSTSSTAESPGPGSTSESPSGTAPGSTSSTAESPGPGSTS | 269 |
| | STAESPGPGTSTPESGSASPGSTSESPSGTAPGTSPSGESSTAPGSTSESPS | |
| | GTAPGSTSESPSGTAPGTSPSGESSTAPGSTSESPSGTAPGSTSESPSGTAP | |
| | GTSPSGESSTAPGSTSESPSGTAPGSTSESPSGTAPGSTSESPSGTAPGTST | |
| | PESGSASPGSTSESPSGTAPGTSTPESGSASPGSTSSTAESPGPGSTSSTAE | |
| | SPGPGTSTPESGSASPGTSTPESGSASPGSTSESPSGTAPGTSTPESGSASP | |
| | GTSTPESGSASPGSTSESPSGTAPGSTSESPSGTAPGSTSESPSGTAPGSTS | |
| | STAESPGPGTSTPESGSASPGTSTPESGSASPGSTSESPSGTAPGSTSESPS | |
| | GTAPGTSTPESGSASPGSTSESPSGTAPGSTSESPSGTAPGTSTPESGSASP | |
| | GTSPSGESSTAPGSTSSTAESPGPGTSPSGESSTAPGSTSSTAESPGPGTST | |
| | PESGSASPGSTSESPSGTAPGSTSSTAESPGPGTSTPESGSASPGTSTPESG | |
| | SASPGSTSSTAESPGPGTSPSGESSTAPGTSTPESGSASPGSTSESPSGTAP | |
| | GSTSESPSGTAPGTSTPESGSASPGTSPSGESSTAPGSTSSTAESPGPGTSP | |
| | SGESSTAPGSTSSTAESPGPGSTSSTAESPGPGTSPSGESSTAPGSTSESPS | |
| | GTAPGSTSESPSGTAPGTSTPESGSASPGSTSESPSGTAPGSTSESPSGTAP | |
| | GTSTPESGSASPGSTSESPSGTAPGSTSESPSGTAPGSTSESPSGTAPGSTS | |
| | STAESPGPGTSPSGESSTAPGTSPSGESSTAP | |
| AG864_2 | GASPGTSSTGSPGSSPSASTGTGPGSSPSASTGTGPGTPGSGTASSSPGSS | 270 |
| | TPSGATGSPGSSPSASTGTGPGASPGTSSTGSPGTPGSGTASSSPGSSTPS | |
| | GATGSPGTPGSGTASSSPGASPGTSSTGSPGASPGTSSTGSPGTPGSGTAS | |
| | SSPGSSTPSGATGSPGASPGTSSTGSPGTPGSGTASSSPGSSTPSGATGSP | |
| | GSSPSASTGTGPGSSPSASTGTGPGSSTPSGATGSPGSSTPSGATGSPGAS | |
| | PGTSSTGSPGASPGTSSTGSPGASPGTSSTGSPGTPGSGTASSSPGASPGT | |
| | SSTGSPGASPGTSSTGSPGASPGTSSTGSPGSSPSASTGTGPGTPGSGTAS | |
| | SSPGASPGTSSTGSPGASPGTSSTGSPGASPGTSSTGSPGSSTPSGATGSP | |
| | GSSTPSGATGSPGASPGTSSTGSPGTPGSGTASSSPGSSTPSGATGSPGSS | |
| | TPSGATGSPGSSTPSGATGSPGSSPSASTGTGPGASPGTSSTGSPGASPGT | |
| | SSTGSPGTPGSGTASSSPGASPGTSSTGSPGASPGTSSTGSPGASPGTSST | |
| | GSPGASPGTSSTGSPGTPGSGTASSSPGSSTPSGATGSPGTPGSGTASSSP | |
| | GSSTPSGATGSPGTPGSGTASSSPGSSTPSGATGSPGSSTPSGATGSPGSS | |
| | PSASTGTGPGSSPSASTGTGPGASPGTSSTGSPGTPGSGTASSSPGSSTPS | |
| | GATGSPGSSPSASTGTGPGSSPSASTGTGPGASPGTSSTGSPGASPGTSST | |
| | GSPGSSTPSGATGSPGSSPSASTGTGPGASPGTSSTGSPGSSPSASTGTGP | |
| | GTPGSGTASSSPGSSTPSGATGSPGSSTPSGATGSPGASPGTSSTGSP | |
| AE108A | TEEGTSESATPESGPGTSTEPSEGSAPGSPAGSPTSTEEGTSTEPSEGSAP | 271 |
| | GTSTEPSEGSAPGTSESATPESGPGSEPATSGSETPGSEPATSGSETPGSP | |
| | AGSPTS | |
| AE108B | GSPAGSPTSTEEGTSTEPSEGSAPGTSESATPESGPGTSESATPESGPGTSE | 272 |
| | SATPESGPGSEPATSGSETPGSEPATSGSETPGSPAGSPTSTEEGTSTEPSE | |
| | GSAP | |
| AE144A | STEPSEGSAPGSPAGSPTSTEEGTSTEPSEGSAPGTSESATPESGPGSEPAT | 273 |
| | SGSETPGTSESATPESGPGSEPATSGSETPGTSESATPESGPGTSTEPSEGS | |
| | APGTSESATPESGPGSPAGSPTSTEEGSPAGSPTSTEEGS | |
| AE144B | SEPATSGSETPGTSESATPESGPGSEPATSGSETPGTSESATPESGPGTSTE | 274 |
| | PSEGSAPGSPAGSPTSTEEGTSESATPESGPGSEPATSGSETPGTSESATPE | |
| | SGPGSPAGSPTSTEEGSPAGSPTSTEEGTSTEPSEGSAPG | |
| AE180A | TSTEEGTSESATPESGPGSEPATSGSETPGTSESATPESGPGSPAGSPTSTE | 275 |
| | EGSPAGSPTSTEEGTSTEPSEGSAPGTSESATPESGPGTSESATPESGPGTS | |
| | ESATPESGPGSEPATSGSETPGSEPATSGSETPGSPAGSPTSTEEGTSTEPS | |
| | EGSAPGTSTEPSEGSAPGSEPATS | |
| AE216A | PESGPGTSTEPSEGSAPGSPAGSPTSTEEGTSESATPESGPGSEPATSGSET | 276 |
| | PGTSESATPESGPGSPAGSPTSTEEGSPAGSPTSTEEGTSTEPSEGSAPGTS | |
| | ESATPESGPGTSESATPESGPGTSESATPESGPGSEPATSGSETPGSEPATS | |
| | GSETPGSPAGSPTSTEEGTSTEPSEGSAPGTSTEPSEGSAPGSEPATSGSET | |
| | PGTSESAT | |
| AE252A | ESGPGSEPATSGSETPGTSESATPESGPGTSTEPSEGSAPGSPAGSPTSTEE | 277 |
| | GTSESATPESGPGSEPATSGSETPGTSESATPESGPGSPAGSPTSTEEGSP | |
| | AGSPTSTEEGTSTEPSEGSAPGTSESATPESGPGTSESATPESGPGTSESA | |
| | TPESGPGSEPATSGSETPGSEPATSGSETPGSPAGSPTSTEEGTSTEPSEGS | |
| | APGTSTEPSEGSAPGSEPATSGSETPGTSESATPESGPGTSTEPSE | |
| AE288A | TPESGPGTSTEPSEGSAPGTSESATPESGPGSEPATSGSETPGTSESATPES | 278 |
| | GPGSEPATSGSETPGTSESATPESGPGTSTEPSEGSAPGSPAGSPTSTEEG | |
| | TSESATPESGPGSEPATSGSETPGTSESATPESGPGSPAGSPTSTEEGSPA | |
| | GSPTSTEEGTSTEPSEGSAPGTSESATPESGPGTSESATPESGPGTSESATP | |
| | ESGPGSEPATSGSETPGSEPATSGSETPGSPAGSPTSTEEGTSTEPSEGSAP | |
| | GTSTEPSEGSAPGSEPATSGSETPGTSESA | |
| AE324A | PESGPGSPAGSPTSTEEGSPAGSPTSTEEGSPAGSPTSTEEGTSESATPESG | 279 |
| | PGTSTEPSEGSAPGTSESATPESGPGSEPATSGSETPGTSESATPESGPGSE | |
| | PATSGSETPGTSESATPESGPGTSTEPSEGSAPGSPAGSPTSTEEGTSESAT | |
| | PESGPGSEPATSGSETPGTSESATPESGPGSPAGSPTSTEEGSPAGSPTSTE | |
| | EGTSTEPSEGSAPGTSESATPESGPGTSESATPESGPGTSESATPESGPGSE | |
| | PATSGSETPGSEPATSGSETPGSPAGSPTSTEEGTSTEPSEGSAPGTSTEPS | |
| | EGSAPGSEPATS | |
| AE360A | PESGPGTSTEPSEGSAPGTSESATPESGPGSPAGSPTSTEEGSPAGSPTSTE | 280 |
| | EGSPAGSPTSTEEGTSESATPESGPGTSTEPSEGSAPGTSESATPESGPGSE | |
| | PATSGSETPGTSESATPESGPGSEPATSGSETPGTSESATPESGPGTSTEPS | |
| | EGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPG | |
| | TSESATPESGPGTSESATPESGPGSEPATSGSETPGSEPATSGSETPGSPA | |
| | GSPTSTEEGTSTEPSEGSAPGTSTEPSEGSAPGSEPATSGSETPGTSESAT | |
| AE396A | PESGPGSEPATSGSETPGTSESATPESGPGTSTEPSEGSAPGTSESATPESG | 281 |
| | PGSPAGSPTSTEEGSPAGSPTSTEEGSPAGSPTSTEEGTSESATPESGPGTS | |
| | TEPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPGTSTEPS | |
| AE432A | EGSAPGSPAGSPTSTEEGTSTEPSEGSAPGTSESATPESGPGSEPATSGSE | 282 |
| | TPGTSESATPESGPGSEPATSGSETPGTSESATPESGPGTSTEPSEGSAPGT | |
| | SESATPESGPGSPAGSPTSTEEGSPAGSPTSTEEGSPAGSPTSTEEGTSESA | |
| | TPESGPGTSTEPSEGSAPGTSESATPESGPGSEPATSGSETPGTSESATPES | |
| | GPGSEPATSGSETPGTSESATPESGPGTSTEPSEGSAPGSPAGSPTSTEEG | |
| | TSESATPESGPGSEPATSGSETPGTSESATPESGPGSPAGSPTSTEEGSPA | |
| | GSPTSTEEGTSTEPSEGSAPGTSESATPESGPGTSESATPESGPGTSESATP | |
| | ESGPGSEPATSGSETPGSEPATSGSETPGSPAGSPTSTEEGTSTEPSEGSAP | |
| | GTSTEPSEGSAPGSEPATS | |
| AE468A | EGSAPGTSTEPSEGSAPGTSTEPSEGSAPGSPAGSPTSTEEGTSTEPSEGS | 283 |
| | APGTSESATPESGPGSEPATSGSETPGTSESATPESGPGSEPATSGSETPG | |
| | TSESATPESGPGTSTEPSEGSAPGTSESATPESGPGSPAGSPTSTEEGSPA | |
| | GSPTSTEEGSPAGSPTSTEEGTSESATPESGPGTSTEPSEGSAPGTSESATP | |
| | ESGPGSEPATSGSETPGTSESATPESGPGSEPATSGSETPGTSESATPESGP | |
| | GTSTEPSEGSAPGSPAGSPTSTEEGTSESATPESGPGSEPATSGSETPGTSE | |
| | SATPESGPGSPAGSPTSTEEGSPAGSPTSTEEGTSTEPSEGSAPGTSESATP | |
| | ESGPGTSESATPESGPGTSESATPESGPGSEPATSGSETPGSEPATSGSETP | |
| | GSPAGSPTSTEEGTSTEPSEGSAPGTSTEPSEGSAPGSEPATSGSETPGTSE | |
| | SAT | |
| AE504A | EGSAPGTSTEPSEGSAPGTSTEPSEGSAPGTSTEPSEGSAPGTSTEPSEGS | 284 |
| | APGSPAGSPTSTEEGTSTEPSEGSAPGTSESATPESGPGSEPATSGSETPG | |
| | TSESATPESGPGSEPATSGSETPGTSESATPESGPGTSTEPSEGSAPGTSES | |
| | ATPESGPGSPAGSPTSTEEGSPAGSPTSTEEGSPAGSPTSTEEGTSESATPE | |
| | SGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSE | |
| | SATPESGPGSEPATSGSETPGTSESATPESGPGSPAGSPTSTEEGSPAGSPT | |
| | STEEGTSTEPSEGSAPGTSESATPESGPGTSESATPESGPGTSESATPESGP | |
| | GSEPATSGSETPGSEPATSGSETPGSPAGSPTSTEEGTSTEPSEGSAPGTST | |
| | EPSEGSAPGSEPATSGSETPGTSESATPESGPGTSTEPS | |
| AE540A | TPESGPGSPAGSPTSTEEGTSESATPESGPGSEPATSGSETPGTSESATPES | 285 |
| | GPGTSTEPSEGSAPGTSTEPSEGSAPGTSTEPSEGSAPGTSTEPSEGSAPG | |
| | TSTEPSEGSAPGTSTEPSEGSAPGSPAGSPTSTEEGTSTEPSEGSAPGTSES | |
| | ATPESGPGSEPATSGSETPGTSESATPESGPGSEPATSGSETPGTSESATPE | |
| | SGPGTSTEPSEGSAPGTSESATPESGPGSPAGSPTSTEEGSPAGSPTSTEE | |
| | GSPAGSPTSTEEGTSESATPESGPGTSTEPSEGSAPGTSESATPESGPGSEP | |
| | ATSGSETPGTSESATPESGPGSEPATSGSETPGTSESATPESGPGTSTEPSE | |
| | GSAPGSPAGSPTSTEEGTSESATPESGPGSEPATSGSETPGTSESATPESG | |
| | PGSPAGSPTSTEEGSPAGSPTSTEEGTSTEPSEGSAPGTSESATPESGPGTS | |
| | ESATPESGPGTSESATPESGPGSEPATSGSETPGSEPATSGSETPGSPAGSP | |
| | TSTEEGTSTEPSEGSAPGTSTEP | |
| AE576A | TPESGPGTSESATPESGPGSPAGSPTSTEEGTSESATPESGPGSEPATSGSE | 286 |
| | TPGTSESATPESGPGTSTEPSEGSAPGTSTEPSEGSAPGTSTEPSEGSAPGT | |
| | STEPSEGSAPGTSTEPSEGSAPGTSTEPSEGSAPGSPAGSPTSTEEGTSTEP | |
| | SEGSAPGTSESATPESGPGSEPATSGSETPGTSESATPESGPGSEPATSGS | |
| | ETPGTSESATPESGPGTSTEPSEGSAPGTSESATPESGPGSPAGSPTSTEEG | |
| | SPAGSPTSTEEGSPAGSPTSTEEGTSESATPESGPGTSTEPSEGSAPGTSES | |
| | ATPESGPGSEPATSGSETPGTSESATPESGPGSEPATSGSETPGTSESATPE | |
| | SGPGTSTEPSEGSAPGSPAGSPTSTEEGTSESATPESGPGSEPATSGSETP | |
| | GTSESATPESGPGSPAGSPTSTEEGSPAGSPTSTEEGTSTEPSEGSAPGTSE | |
| | SATPESGPGTSESATPESGPGTSESATPESGPGSEPATSGSETPGSEPATS | |
| | GSETPGSPAGSPTSTEEGTSTEPSEGSAPGTSTEPSEGSAPGSEPATSGSET | |
| | PGTSESA | |
| AE612A | GSETPGTSTEPSEGSAPGTSTEPSEGSAPGTSESATPESGPGTSESATPESG | 287 |
| | PGSPAGSPTSTEEGTSESATPESGPGSEPATSGSETPGTSESATPESGPGTS | |
| | TEPSEGSAPGTSTEPSEGSAPGTSTEPSEGSAPGTSTEPSEGSAPGTSTEPS | |
| | EGSAPGTSTEPSEGSAPGSPAGSPTSTEEGTSTEPSEGSAPGTSESATPES | |
| | GPGSEPATSGSETPGTSESATPESGPGSEPATSGSETPGTSESATPESGPG | |
| | TSTEPSEGSAPGTSESATPESGPGSPAGSPTSTEEGSPAGSPTSTEEGSPA | |
| | GSPTSTEEGTSESATPESGPGTSTEPSEGSAPGTSESATPESGPGSEPATSG | |
| | SETPGTSESATPESGPGSEPATSGSETPGTSESATPESGPGTSTEPSEGSAP | |
| | GSPAGSPTSTEEGTSESATPESGPGSEPATSGSETPGTSESATPESGPGSP | |
| | AGSPTSTEEGSPAGSPTSTEEGTSTEPSEGSAPGTSESATPESGPGTSESA | |
| | TPESGPGTSESATPESGPGSEPATSGSETPGSEPATSGSETPGSPAGSPTST | |
| | EEGTSTEPSEGSAPGTSTEPSEGSAPGSEPATSGSETPGTSESAT | |
| AE648A | PESGPGTSTEPSEGSAPGTSESATPESGPGSEPATSGSETPGTSTEPSEGSA | 288 |
| | PGTSTEPSEGSAPGTSESATPESGPGTSESATPESGPGSPAGSPTSTEEGTS | |
| | ESATPESGPGSEPATSGSETPGTSESATPESGPGTSTEPSEGSAPGTSTEPS | |
| | EGSAPGTSTEPSEGSAPGTSTEPSEGSAPGTSTEPSEGSAPGTSTEPSEGS | |
| | APGSPAGSPTSTEEGTSTEPSEGSAPGTSESATPESGPGSEPATSGSETPG | |
| | TSESATPESGPGSEPATSGSETPGTSESATPESGPGTSTEPSEGSAPGTSES | |
| | ATPESGPGSPAGSPTSTEEGSPAGSPTSTEEGSPAGSPTSTEEGTSESATPE | |
| | SGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSE | |
| | SATPESGPGSEPATSGSETPGTSESATPESGPGSPAGSPTSTEEGSPAGSPT | |
| | STEEGTSTEPSEGSAPGTSESATPESGPGTSESATPESGPGTSESATPESGP | |
| | GSEPATSGSETPGSEPATSGSETPGSPAGSPTSTEEGTSTEPSEGSAPGTST | |
| | EPSEGSAPGSEPATSGSETPGTSESAT | |
| AE684A | EGSAPGSPAGSPTSTEEGTSTEPSEGSAPGTSTEPSEGSAPGTSESATPES | 289 |
| | GPGTSTEPSEGSAPGTSESATPESGPGSEPATSGSETPGTSTEPSEGSAPG | |
| | TSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEG | |
| | SAPGTSTEPSEGSAPGTSTEPSEGSAPGTSTEPSEGSAPGTSTEPSEGSAP | |
| | GSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATP | |
| | ESGPGSPAGSPTSTEEGSPAGSPTSTEEGSPAGSPTSTEEGTSESATPESGP | |
| | GTSTEPSEGSAPGTSESATPESGPGSEPATSGSETPGTSESATPESGPGSEP | |
| | ATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEE | |
| | GTSTEPSEGSAPGTSESATPESGPGTSESATPESGPGTSESATPESGPGSEP | |
| | ATSGSETPGSEPATSGSETPGSPAGSPTSTEEGTSTEPSEGSAPGTSTEPSE | |
| | GSAPGSEPATS | |
| AE720A | TSGSETPGSEPATSGSETPGSPAGSPTSTEEGTSESATPESGPGTSTEPSEG | 290 |
| | SAPGTSTEPSEGSAPGSPAGSPTSTEEGTSTEPSEGSAPGTSTEPSEGSAP | |
| | GTSESATPESGPGTSTEPSEGSAPGTSESATPESGPGSEPATSGSETPGTST | |
| | EPSEGSAPGTSTEPSEGSAPGTSESATPESGPGTSESATPESGPGSPAGSPT | |
| | STEEGTSESATPESGPGSEPATSGSETPGTSESATPESGPGTSTEPSEGSAP | |
| | GTSTEPSEGSAPGTSTEPSEGSAPGTSTEPSEGSAPGTSTEPSEGSAPGTST | |
| | EPSEGSAPGSPAGSPTSTEEGTSTEPSEGSAPGTSESATPESGPGSEPATS | |
| | GSETPGTSESATPESGPGSEPATSGSETPGTSESATPESGPGTSTEPSEGSA | |
| | PGTSESATPESGPGSPAGSPTSTEEGSPAGSPTSTEEGSPAGSPTSTEEGTS | |
| | ESATPESGPGTSTEPSEGSAPGTSESATPESGPGSEPATSGSETPGTSESAT | |
| | PESGPGSEPATSGSETPGTSESATPESGPGTSTEPSEGSAPGSPAGSPTSTE | |
| | EGTSESATPESGPGSEPATSGSETPGTSESATPESGPGSPAGSPTSTEEGSP | |
| | AGSPTSTEEGTSTEPSEGSAPGTSESATPESGPGTSESATPESGPGTSESA | |
| | TPESGPGSEPATSGSETPGSEPATSGSETPGSPAGSPTSTEEGTSTE | |
| AE756A | TSGSETPGSEPATSGSETPGSPAGSPTSTEEGTSESATPESGPGTSTEPSEG | 291 |
| | SAPGTSTEPSEGSAPGSPAGSPTSTEEGTSTEPSEGSAPGTSTEPSEGSAP | |
| | GTSESATPESGPGTSTEPSEGSAPGTSESATPESGPGSEPATSGSETPGTST | |
| | EPSEGSAPGTSTEPSEGSAPGTSESATPESGPGTSESATPESGPGSPAGSPT | |
| | STEEGTSESATPESGPGSEPATSGSETPGTSESATPESGPGTSTEPSEGSAP | |
| | GTSTEPSEGSAPGTSTEPSEGSAPGTSTEPSEGSAPGTSTEPSEGSAPGTST | |
| | EPSEGSAPGSPAGSPTSTEEGTSTEPSEGSAPGTSESATPESGPGSEPATS | |
| | GSETPGTSESATPESGPGSEPATSGSETPGTSESATPESGPGTSTEPSEGSA | |
| | PGTSESATPESGPGSPAGSPTSTEEGSPAGSPTSTEEGSPAGSPTSTEEGTS | |
| | ESATPESGPGTSTEPSEGSAPGTSESATPESGPGSEPATSGSETPGTSESAT | |
| | PESGPGSEPATSGSETPGTSESATPESGPGTSTEPSEGSAPGSPAGSPTSTE | |
| | EGTSESATPESGPGSEPATSGSETPGTSESATPESGPGSPAGSPTSTEEGSP | |
| | AGSPTSTEEGTSTEPSEGSAPGTSESATPESGPGTSESATPESGPGTSESA | |
| | TPESGPGSEPATSGSETPGSEPATSGSETPGSPAGSPTSTEEGTSTEPSEGS | |
| | APGTSTEPSEGSAPGSEPATSGSETPGTSES | |
| AE792A | EGSAPGTSESATPESGPGSEPATSGSETPGSEPATSGSETPGSPAGSPTST | 292 |
| | EEGTSESATPESGPGTSTEPSEGSAPGTSTEPSEGSAPGSPAGSPTSTEEG | |
| | TSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPE | |
| | SGPGTSESATPESGPGSPAGSPTSTEEGTSESATPESGPGSEPATSGSETP | |
| | GTSESATPESGPGTSTEPSEGSAPGTSTEPSEGSAPGTSTEPSEGSAPGTST | |
| | EPSEGSAPGTSTEPSEGSAPGTSTEPSEGSAPGSPAGSPTSTEEGTSTEPSE | |
| | GSAPGTSESATPESGPGSEPATSGSETPGTSESATPESGPGSEPATSGSET | |
| | PGTSESATPESGPGTSTEPSEGSAPGTSESATPESGPGSPAGSPTSTEEGSP | |
| | AGSPTSTEEGSPAGSPTSTEEGTSESATPESGPGTSTEPSEGSAPGTSESA | |
| | TPESGPGSEPATSGSETPGTSESATPESGPGSEPATSGSETPGTSESATPES | |
| | GPGTSTEPSEGSAPGSPAGSPTSTEEGTSESATPESGPGSEPATSGSETPG | |
| | TSESATPESGPGSPAGSPTSTEEGSPAGSPTSTEEGTSTEPSEGSAPGTSES | |
| | ATPESGPGTSESATPESGPGTSESATPESGPGSEPATSGSETPGSEPATSG | |
| | SETPGSPAGSPTSTEEGTSTEPSEGSAPGTSTEPSEGSAPGSEPATSGSETP | |
| | GTSESATPESGPGTSTEPS | |
| AE828A | PESGPGTSTEPSEGSAPGSPAGSPTSTEEGTSTEPSEGSAPGTSTEPSEGSA | 293 |
| | PGTSESATPESGPGSEPATSGSETPGSEPATSGSETPGSPAGSPTSTEEGTS | |
| | ESATPESGPGTSTEPSEGSAPGTSTEPSEGSAPGSPAGSPTSTEEGTSTEPS | |
| | EGSAPGTSTEPSEGSAPGTSESATPESGPGTSTEPSEGSAPGTSESATPES | |
| | GPGSEPATSGSETPGTSTEPSEGSAPGTSTEPSEGSAPGTSESATPESGPG | |
| | TSESATPESGPGSPAGSPTSTEEGTSESATPESGPGSEPATSGSETPGTSES | |
| | ATPESGPGTSTEPSEGSAPGTSTEPSEGSAPGTSTEPSEGSAPGTSTEPSEG | |
| | SAPGTSTEPSEGSAPGTSTEPSEGSAPGSPAGSPTSTEEGTSTEPSEGSAP | |
| | GTSESATPESGPGSEPATSGSETPGTSESATPESGPGSEPATSGSETPGTSE | |
| | SATPESGPGTSTEPSEGSAPGTSESATPESGPGSPAGSPTSTEEGSPAGSPT | |
| | STEEGSPAGSPTSTEEGTSESATPESGPGTSTEPSEGSAPGTSESATPESGP | |
| | GSEPATSGSETPGTSESATPESGPGSEPATSGSETPGTSESATPESGPGTST | |
| | EPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGP | |
| | GTSESATPESGPGTSESATPESGPGSEPATSGSETPGSEPATSGSETPGSP | |
| | AGSPTSTEEGTSTEPSEGSAPGTSTEPSEGSAPGSEPATSGSETPGTSESA | |
| | T | |
| AG108A | SASTGTGPGSSPSASTGTGPGTPGSGTASSSPGSSTPSGATGSPGSSPSAS | 294 |
| | TGTGPGASPGTSSTGSPGTPGSGTASSSPGSSTPSGATGSPGTPGSGTASS | |
| | SPGASP | |
| AG108B | PGTPGSGTASSSPGSSTPSGATGSPGTPGSGTASSSPGSSTPSGATGSPGS | 295 |
| | STPSGATGSPGSSPSASTGTGPGSSPSASTGTGPGASPGTSSTGSPGTPGS | |
| | GTASSS | |
| AG144A | PGSSPSASTGTGPGSSPSASTGTGPGTPGSGTASSSPGSSTPSGATGSPGS | 296 |
| | SPSASTGTGPGASPGTSSTGSPGTPGSGTASSSPGSSTPSGATGSPGTPGS | |
| | GTASSSPGASPGTSSTGSPGASPGTSSTGSPGTPGSGTASSS | |
| AG144B | PSGATGSPGTPGSGTASSSPGSSTPSGATGSPGSSTPSGATGSPGSSPSAS | 297 |
| | TGTGPGSSPSASTGTGPGASPGTSSTGSPGTPGSGTASSSPGSSTPSGATG | |
| | SPGSSPSASTGTGPGSSPSASTGTGPGASPGTSSTGSPGASP | |
| AG180A | TSSTGSPGSSPSASTGTGPGSSPSASTGTGPGTPGSGTASSSPGSSTPSGA | 298 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGS | |
| AG216A | TGTGPGSSPSASTGTGPGTPGSGTASSSPGSSTPSGATGSPGSSPSASTGT | 299 |
| | GPGASPGTSSTGSPGTPGSGTASSSPGSSTPSGATGSPGTPGSGTASSSPG | |
| | ASPGTSSTGSPGASPGTSSTGSPGTPGSGTASSSPGSSTPSGATGSPGASP | |
| | GTSSTGSPGTPGSGTASSSPGSSTPSGATGSPGSSPSASTGTGPGSSPSAST | |
| | GTGPGSSTPSG | |
| AG252A | TSSTGSPGSSPSASTGTGPGSSPSASTGTGPGTPGSGTASSSPGSSTPSGA | 300 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGSGTASSSPGSSTPSGATGSPGSSPS | |
| | ASTGTGPGSSPSASTGTGPGSSTPSGATGSPGSSTPSGATGSPGASPG | |
| AG288A | TSSTGSPGSSPSASTGTGPGSSPSASTGTGPGTPGSGTASSSPGSSTPSGA | 301 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGSGTASSSPGSSTPSGATGSPGSSPS | |
| | ASTGTGPGSSPSASTGTGPGSSTPSGATGSPGSSTPSGATGSPGASPGTSS | |
| | TGSPGASPGTSSTGSPGASPGTSSTGSPGTPGS | |
| AG324A | TSSTGSPGTPGSGTASSSPGASPGTSSTGSPGASPGTSSTGSPGASPGTSS | 302 |
| | TGSPGASPGTSSTGSPGTPGSGTASSSPGSSTPSGATGSPGTPGSGTASSS | |
| | PGSSTPSGATGSPGTPGSGTASSSPGSSTPSGATGSPGSSTPSGATGSPGS | |
| | SPSASTGTGPGSSPSASTGTGPGASPGTSSTGSPGTPGSGTASSSPGSSTPS | |
| | GATGSPGSSPSASTGTGPGSSPSASTGTGPGASPGTSSTGSPGASPGTSST | |
| | GSPGSSTPSGATGSPGSSPSASTGTGPGASPGTSSTGSPGSSPSASTGTGP | |
| | GTPGSGTASSSPGSSTP | |
| AG360A | TSSTGSPGASPGTSSTGSPGTPGSGTASSSPGASPGTSSTGSPGASPGTSS | 303 |
| | TGSPGASPGTSSTGSPGASPGTSSTGSPGTPGSGTASSSPGSSTPSGATGS | |
| | PGTPGSGTASSSPGSSTPSGATGSPGTPGSGTASSSPGSSTPSGATGSPGS | |
| | STPSGATGSPGSSPSASTGTGPGSSPSASTGTGPGASPGTSSTGSPGTPGS | |
| | GTASSSPGSSTPSGATGSPGSSPSASTGTGPGSSPSASTGTGPGASPGTSS | |
| | TGSPGASPGTSSTGSPGSSTPSGATGSPGSSPSASTGTGPGASPGTSSTGS | |
| | PGSSPSASTGTGPGTPGSGTASSSPGSSTPSGATGSPGSSTPSGATGSPGA | |
| | SPG | |
| AG396A | GATGSPGSSTPSGATGSPGSSPSASTGTGPGASPGTSSTGSPGASPGTSST | 304 |
| | GSPGTPGSGTASSSPGASPGTSSTGSPGASPGTSSTGSPGASPGTSSTGSP | |
| | GASPGTSSTGSPGTPGSGTASSSPGSSTPSGATGSPGTPGSGTASSSPGSS | |
| | TPSGATGSPGTPGSGTASSSPGSSTPSGATGSPGSSTPSGATGSPGSSPSA | |
| | STGTGPGSSPSASTGTGPGASPGTSSTGSPGTPGSGTASSSPGSSTPSGAT | |
| | GSPGSSPSASTGTGPGSSPSASTGTGPGASPGTSSTGSPGASPGTSSTGSP | |
| | GSSTPSGATGSPGSSPSASTGTGPGASPGTSSTGSPGSSPSASTGTGPGTP | |
| | GSGTASSSPGSSTPSGATGSPGSSTPSGATGSPGASPGT | |
| AG432A | GATGSPGSSTPSGATGSPGASPGTSSTGSPGTPGSGTASSSPGSSTPSGAT | 305 |
| | GSPGSSTPSGATGSPGSSTPSGATGSPGSSPSASTGTGPGASPGTSSTGSP | |
| | GASPGTSSTGSPGTPGSGTASSSPGASPGTSSTGSPGASPGTSSTGSPGAS | |
| | PGTSSTGSPGASPGTSSTGSPGTPGSGTASSSPGSSTPSGATGSPGTPGSG | |
| | TASSSPGSSTPSGATGSPGTPGSGTASSSPGSSTPSGATGSPGSSTPSGAT | |
| | GSPGSSPSASTGTGPGSSPSASTGTGPGASPGTSSTGSPGTPGSGTASSSP | |
| | GSSTPSGATGSPGSSPSASTGTGPGSSPSASTGTGPGASPGTSSTGSPGAS | |
| | PGTSSTGSPGSSTPSGATGSPGSSPSASTGTGPGASPGTSSTGSPGSSPSAS | |
| | TGTGPGTPGSGTASSSPGSSTPS | |
| AG468A | TSSTGSPGSSPSASTGTGPGTPGSGTASSSPGASPGTSSTGSPGASPGTSS | 306 |
| | TGSPGASPGTSSTGSPGSSTPSGATGSPGSSTPSGATGSPGASPGTSSTGS | |
| | PGTPGSGTASSSPGSSTPSGATGSPGSSTPSGATGSPGSSTPSGATGSPGS | |
| | SPSASTGTGPGASPGTSSTGSPGASPGTSSTGSPGTPGSGTASSSPGASPG | |
| | TSSTGSPGASPGTSSTGSPGASPGTSSTGSPGASPGTSSTGSPGTPGSGTA | |
| | SSSPGSSTPSGATGSPGTPGSGTASSSPGSSTPSGATGSPGTPGSGTASSSP | |
| | GSSTPSGATGSPGSSTPSGATGSPGSSPSASTGTGPGSSPSASTGTGPGAS | |
| | PGTSSTGSPGTPGSGTASSSPGSSTPSGATGSPGSSPSASTGTGPGSSPSAS | |
| | TGTGPGASPGTSSTGSPGASPGTSSTGSPGSSTPSGATGSPGSSPSASTGT | |
| | GPGASPG | |
| AGS04A | TSSTGSPGSSPSASTGTGPGTPGSGTASSSPGASPGTSSTGSPGASPGTSS | 307 |
| | TGSPGASPGTSSTGSPGSSTPSGATGSPGSSTPSGATGSPGASPGTSSTGS | |
| | PGTPGSGTASSSPGSSTPSGATGSPGSSTPSGATGSPGSSTPSGATGSPGS | |
| | SPSASTGTGPGASPGTSSTGSPGASPGTSSTGSPGTPGSGTASSSPGASPG | |
| | TSSTGSPGASPGTSSTGSPGASPGTSSTGSPGASPGTSSTGSPGTPGSGTA | |
| | SSSPGSSTPSGATGSPGTPGSGTASSSPGSSTPSGATGSPGTPGSGTASSSP | |
| | GSSTPSGATGSPGSSTPSGATGSPGSSPSASTGTGPGSSPSASTGTGPGAS | |
| | PGTSSTGSPGTPGSGTASSSPGSSTPSGATGSPGSSPSASTGTGPGSSPSAS | |
| | TGTGPGASPGTSSTGSPGASPGTSSTGSPGSSTPSGATGSPGSSPSASTGT | |
| | GPGASPGTSSTGSPGSSPSASTGTGPGTPGSGTASSSPGSSTP | |
| AG540A | TSSTGSPGASPGTSSTGSPGSSPSASTGTGPGTPGSGTASSSPGASPGTSS | 308 |
| | TGSPGASPGTSSTGSPGASPGTSSTGSPGSSTPSGATGSPGSSTPSGATGS | |
| | PGASPGTSSTGSPGTPGSGTASSSPGSSTPSGATGSPGSSTPSGATGSPGS | |
| | STPSGATGSPGSSPSASTGTGPGASPGTSSTGSPGASPGTSSTGSPGTPGS | |
| | GTASSSPGASPGTSSTGSPGASPGTSSTGSPGASPGTSSTGSPGASPGTSS | |
| | TGSPGTPGSGTASSSPGSSTPSGATGSPGTPGSGTASSSPGSSTPSGATGS | |
| | PGTPGSGTASSSPGSSTPSGATGSPGSSTPSGATGSPGSSPSASTGTGPGS | |
| | SPSASTGTGPGASPGTSSTGSPGTPGSGTASSSPGSSTPSGATGSPGSSPS | |
| | ASTGTGPGSSPSASTGTGPGASPGTSSTGSPGASPGTSSTGSPGSSTPSGA | |
| | TGSPGSSPSASTGTGPGASPGTSSTGSPGSSPSASTGTGPGTPGSGTASSS | |
| | PGSSTPSGATGSPGSSTPSGATGSPGASPG | |
| AG576A | TSSTGSPGTPGSGTASSSPGASPGTSSTGSPGASPGTSSTGSPGASPGTSS | 309 |
| | TGSPGSSPSASTGTGPGTPGSGTASSSPGASPGTSSTGSPGASPGTSSTGS | |
| | PGASPGTSSTGSPGSSTPSGATGSPGSSTPSGATGSPGASPGTSSTGSPGT | |
| | PGSGTASSSPGSSTPSGATGSPGSSTPSGATGSPGSSTPSGATGSPGSSPS | |
| | ASTGTGPGASPGTSSTGSPGASPGTSSTGSPGTPGSGTASSSPGASPGTSS | |
| | TGSPGASPGTSSTGSPGASPGTSSTGSPGASPGTSSTGSPGTPGSGTASSS | |
| | PGSSTPSGATGSPGTPGSGTASSSPGSSTPSGATGSPGTPGSGTASSSPGS | |
| | STPSGATGSPGSSTPSGATGSPGSSPSASTGTGPGSSPSASTGTGPGASPG | |
| | TSSTGSPGTPGSGTASSSPGSSTPSGATGSPGSSPSASTGTGPGSSPSASTG | |
| | TGPGASPGTSSTGSPGASPGTSSTGSPGSSTPSGATGSPGSSPSASTGTGP | |
| | GASPGTSSTGSPGSSPSASTGTGPGTPGSGTASSSPGSSTPSGATGSPGSS | |
| | TPSGATGSPGASPG | |
| AG612A | STGSPGTPGSGTASSSPGSSTPSGATGSPGASPGTSSTGSPGTPGSGTASS | 310 |
| | SPGSSTPSGATGSPGSSPSASTGTGPGSSPSASTGTGPGSSTPSGATGSPG | |
| | SSTPSGATGSPGASPGTSSTGSPGASPGTSSTGSPGASPGTSSTGSPGTPG | |
| | SGTASSSPGASPGTSSTGSPGASPGTSSTGSPGASPGTSSTGSPGSSPSAST | |
| | GTGPGTPGSGTASSSPGASPGTSSTGSPGASPGTSSTGSPGASPGTSSTGS | |
| | PGSSTPSGATGSPGSSTPSGATGSPGASPGTSSTGSPGTPGSGTASSSPGS | |
| | STPSGATGSPGSSTPSGATGSPGSSTPSGATGSPGSSPSASTGTGPGASPG | |
| | TSSTGSPGASPGTSSTGSPGTPGSGTASSSPGASPGTSSTGSPGASPGTSS | |
| | TGSPGASPGTSSTGSPGASPGTSSTGSPGTPGSGTASSSPGSSTPSGATGS | |
| | PGTPGSGTASSSPGSSTPSGATGSPGTPGSGTASSSPGSSTPSGATGSPGS | |
| | STPSGATGSPGSSPSASTGTGPGSSPSASTGTGPGASPGTSSTGSPGTPGS | |
| | GTASSSPGSSTPSGATGSPGSSPSASTGTGPGSSPSASTGTGPGASPGTS | |
| AG648A | GTASSSPGSSTPSGATGSPGSSPSASTGTGPGSSPSASTGTGPGSSTPSGA | 311 |
| | TGSPGSSTPSGATGSPGASPGTSSTGSPGASPGTSSTGSPGASPGTSSTGS | |
| | PGTPGSGTASSSPGASPGTSSTGSPGASPGTSSTGSPGASPGTSSTGSPGS | |
| | SPSASTGTGPGTPGSGTASSSPGASPGTSSTGSPGASPGTSSTGSPGASPG | |
| | TSSTGSPGSSTPSGATGSPGSSTPSGATGSPGASPGTSSTGSPGTPGSGTA | |
| | SSSPGSSTPSGATGSPGSSTPSGATGSPGSSTPSGATGSPGSSPSASTGTGP | |
| | GASPGTSSTGSPGASPGTSSTGSPGTPGSGTASSSPGASPGTSSTGSPGAS | |
| | PGTSSTGSPGASPGTSSTGSPGASPGTSSTGSPGTPGSGTASSSPGSSTPSG | |
| | ATGSPGTPGSGTASSSPGSSTPSGATGSPGTPGSGTASSSPGSSTPSGATG | |
| | SPGSSTPSGATGSPGSSPSASTGTGPGSSPSASTGTGPGASPGTSSTGSPG | |
| | TPGSGTASSSPGSSTPSGATGSPGSSPSASTGTGPGSSPSASTGTGPGASP | |
| | GTSSTGSPGASPGTSSTGSPGSSTPSGATGSPGSSPSASTGTGPGASPGTS | |
| | STGSPGSSPSASTGTGPGTPGSGTASSSPGSSTP | |
| AG684A | TSSTGSPGTPGSGTASSSPGSSTPSGATGSPGSSPSASTGTGPGSSPSASTG | 312 |
| | TGPGSSTPSGATGSPGSSTPSGATGSPGASPGTSSTGSPGASPGTSSTGSP | |
| | GASPGTSSTGSPGTPGSGTASSSPGASPGTSSTGSPGASPGTSSTGSPGAS | |
| | PGTSSTGSPGSSPSASTGTGPGTPGSGTASSSPGASPGTSSTGSPGASPGT | |
| | SSTGSPGASPGTSSTGSPGSSTPSGATGSPGSSTPSGATGSPGASPGTSST | |
| | GSPGTPGSGTASSSPGSSTPSGATGSPGSSTPSGATGSPGSSTPSGATGSP | |
| | GSSPSASTGTGPGASPGTSSTGSPGASPGTSSTGSPGTPGSGTASSSPGAS | |
| | PGTSSTGSPGASPGTSSTGSPGASPGTSSTGSPGASPGTSSTGSPGTPGSG | |
| | TASSSPGSSTPSGATGSPGTPGSGTASSSPGSSTPSGATGSPGTPGSGTAS | |
| | SSPGSSTPSGATGSPGSSTPSGATGSPGSSPSASTGTGPGSSPSASTGTGP | |
| | GASPGTSSTGSPGTPGSGTASSSPGSSTPSGATGSPGSSPSASTGTGPGSS | |
| | PSASTGTGPGASPGTSSTGSPGASPGTSSTGSPGSSTPSGATGSPGSSPSA | |
| | STGTGPGASPGTSSTGSPGSSPSASTGTGPGTPGSGTASSSPGSSTPSGAT | |
| | GSPGSSTPSGATGSPGASPG | |
| AG720A | TSSTGSPGTPGSGTASSSPGSSTPSGATGSPGASPGTSSTGSPGTPGSGTA | 313 |
| | SSSPGSSTPSGATGSPGSSPSASTGTGPGSSPSASTGTGPGSSTPSGATGSP | |
| | GSSTPSGATGSPGASPGTSSTGSPGASPGTSSTGSPGASPGTSSTGSPGTP | |
| | GSGTASSSPGASPGTSSTGSPGASPGTSSTGSPGASPGTSSTGSPGSSPSA | |
| | STGTGPGTPGSGTASSSPGASPGTSSTGSPGASPGTSSTGSPGASPGTSST | |
| | GSPGSSTPSGATGSPGSSTPSGATGSPGASPGTSSTGSPGTPGSGTASSSP | |
| | GSSTPSGATGSPGSSTPSGATGSPGSSTPSGATGSPGSSPSASTGTGPGAS | |
| | PGTSSTGSPGASPGTSSTGSPGTPGSGTASSSPGASPGTSSTGSPGASPGT | |
| | SSTGSPGASPGTSSTGSPGASPGTSSTGSPGTPGSGTASSSPGSSTPSGAT | |
| | GSPGTPGSGTASSSPGSSTPSGATGSPGTPGSGTASSSPGSSTPSGATGSP | |
| | GSSTPSGATGSPGSSPSASTGTGPGSSPSASTGTGPGASPGTSSTGSPGTP | |
| | GSGTASSSPGSSTPSGATGSPGSSPSASTGTGPGSSPSASTGTGPGASPGT | |
| | SSTGSPGASPGTSSTGSPGSSTPSGATGSPGSSPSASTGTGPGASPGTSST | |
| | GSPGSSPSASTGTGPGTPGSGTASSSPGSSTPSGATGSPGSSTPSGATGSP | |
| | GASPG | |
| AG756A | TSSTGSPGSSPSASTGTGPGSSPSASTGTGPGTPGSGTASSSPGSSTPSGA | 314 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGSGTASSSPGSSTPSGATGSPGSSPS | |
| | ASTGTGPGSSPSASTGTGPGSSTPSGATGSPGSSTPSGATGSPGASPGTSS | |
| | TGSPGASPGTSSTGSPGASPGTSSTGSPGTPGSGTASSSPGASPGTSSTGS | |
| | PGASPGTSSTGSPGASPGTSSTGSPGSSPSASTGTGPGTPGSGTASSSPGA | |
| | SPGTSSTGSPGASPGTSSTGSPGASPGTSSTGSPGSSTPSGATGSPGSSTPS | |
| | GATGSPGASPGTSSTGSPGTPGSGTASSSPGSSTPSGATGSPGSSTPSGAT | |
| | GSPGSSTPSGATGSPGSSPSASTGTGPGASPGTSSTGSPGASPGTSSTGSP | |
| | GTPGSGTASSSPGASPGTSSTGSPGASPGTSSTGSPGASPGTSSTGSPGAS | |
| | PGTSSTGSPGTPGSGTASSSPGSSTPSGATGSPGTPGSGTASSSPGSSTPSG | |
| | ATGSPGTPGSGTASSSPGSSTPSGATGSPGSSTPSGATGSPGSSPSASTGT | |
| | GPGSSPSASTGTGPGASPGTSSTGSPGTPGSGTASSSPGSSTPSGATGSPG | |
| | SSPSASTGTGPGSSPSASTGTGPGASPGTSSTGSPGASPG | |
| AG792A | TSSTGSPGSSPSASTGTGPGSSPSASTGTGPGTPGSGTASSSPGSSTPSGA | 315 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGSGTASSSPGSSTPSGATGSPGSSPS | |
| | ASTGTGPGSSPSASTGTGPGSSTPSGATGSPGSSTPSGATGSPGASPGTSS | |
| | TGSPGASPGTSSTGSPGASPGTSSTGSPGTPGSGTASSSPGASPGTSSTGS | |
| | PGASPGTSSTGSPGASPGTSSTGSPGSSPSASTGTGPGTPGSGTASSSPGA | |
| | SPGTSSTGSPGASPGTSSTGSPGASPGTSSTGSPGSSTPSGATGSPGSSTPS | |
| | GATGSPGASPGTSSTGSPGTPGSGTASSSPGSSTPSGATGSPGSSTPSGAT | |
| | GSPGSSTPSGATGSPGSSPSASTGTGPGASPGTSSTGSPGASPGTSSTGSP | |
| | GTPGSGTASSSPGASPGTSSTGSPGASPGTSSTGSPGASPGTSSTGSPGAS | |
| | PGTSSTGSPGTPGSGTASSSPGSSTPSGATGSPGTPGSGTASSSPGSSTPSG | |
| | ATGSPGTPGSGTASSSPGSSTPSGATGSPGSSTPSGATGSPGSSPSASTGT | |
| | GPGSSPSASTGTGPGASPGTSSTGSPGTPGSGTASSSPGSSTPSGATGSPG | |
| | SSPSASTGTGPGSSPSASTGTGPGASPGTSSTGSPGASPGTSSTGSPGSSTP | |
| | SGATGSPGSSPSASTGTGPGASPG | |
| AG828A | TSSTGSPGSSPSASTGTGPGSSPSASTGTGPGTPGSGTASSSPGSSTPSGA | 316 |
| | TGSPGSSPSASTGTGPGASPGTSSTGSPGTPGSGTASSSPGSSTPSGATGS | |
| | PGTPGSGTASSSPGASPGTSSTGSPGASPGTSSTGSPGTPGSGTASSSPGS | |
| | STPSGATGSPGASPGTSSTGSPGTPGSGTASSSPGSSTPSGATGSPGSSPS | |
| | ASTGTGPGSSPSASTGTGPGSSTPSGATGSPGSSTPSGATGSPGASPGTSS | |
| | TGSPGASPGTSSTGSPGASPGTSSTGSPGTPGSGTASSSPGASPGTSSTGS | |
| | PGASPGTSSTGSPGASPGTSSTGSPGSSPSASTGTGPGTPGSGTASSSPGA | |
| | SPGTSSTGSPGASPGTSSTGSPGASPGTSSTGSPGSSTPSGATGSPGSSTPS | |
| | GATGSPGASPGTSSTGSPGTPGSGTASSSPGSSTPSGATGSPGSSTPSGAT | |
| | GSPGSSTPSGATGSPGSSPSASTGTGPGASPGTSSTGSPGASPGTSSTGSP | |
| | GTPGSGTASSSPGASPGTSSTGSPGASPGTSSTGSPGASPGTSSTGSPGAS | |
| | PGTSSTGSPGTPGSGTASSSPGSSTPSGATGSPGTPGSGTASSSPGSSTPSG | |
| | ATGSPGTPGSGTASSSPGSSTPSGATGSPGSSTPSGATGSPGSSPSASTGT | |
| | GPGSSPSASTGTGPGASPGTSSTGSPGTPGSGTASSSPGSSTPSGATGSPG | |
| | SSPSASTGTGPGSSPSASTGTGPGASPGTSSTGSPGASPGTSSTGSPGSSTP | |
| | SGATGSPGSSPSASTGTGPGASPGTSSTGSPGSSPSASTGTGPGTPGSGTA | |
| | SSSPGSSTP | |
| AE869 | GSPGSPAGSPTSTEEGTSESATPESGPGTSTEPSEGSAPGSPAGSPTSTEE | 317 |
| | GTSTEPSEGSAPGTSTEPSEGSAPGTSESATPESGPGSEPATSGSETPGSEP | |
| | ATSGSETPGSPAGSPTSTEEGTSESATPESGPGTSTEPSEGSAPGTSTEPSE | |
| | GSAPGSPAGSPTSTEEGTSTEPSEGSAPGTSTEPSEGSAPGTSESATPESG | |
| | PGTSTEPSEGSAPGTSESATPESGPGSEPATSGSETPGTSTEPSEGSAPGTS | |
| | TEPSEGSAPGTSESATPESGPGTSESATPESGPGSPAGSPTSTEEGTSESAT | |
| | PESGPGSEPATSGSETPGTSESATPESGPGTSTEPSEGSAPGTSTEPSEGSA | |
| | PGTSTEPSEGSAPGTSTEPSEGSAPGTSTEPSEGSAPGTSTEPSEGSAPGSP | |
| | AGSPTSTEEGTSTEPSEGSAPGTSESATPESGPGSEPATSGSETPGTSESA | |
| | TPESGPGSEPATSGSETPGTSESATPESGPGTSTEPSEGSAPGTSESATPES | |
| | GPGSPAGSPTSTEEGSPAGSPTSTEEGSPAGSPTSTEEGTSESATPESGPG | |
| | TSTEPSEGSAPGTSESATPESGPGSEPATSGSETPGTSESATPESGPGSEPA | |
| | TSGSETPGTSESATPESGPGTSTEPSEGSAPGSPAGSPTSTEEGTSESATPE | |
| | SGPGSEPATSGSETPGTSESATPESGPGSPAGSPTSTEEGSPAGSPTSTEE | |
| | GTSTEPSEGSAPGTSESATPESGPGTSESATPESGPGTSESATPESGPGSEP | |
| | ATSGSETPGSEPATSGSETPGSPAGSPTSTEEGTSTEPSEGSAPGTSTEPSE | |
| | GSAPGSEPATSGSETPGTSESATPESGPGTSTEPSEGSAPGR | |
| AE144_R1 | SAGSPGSPAGSPTSTEEGTSESATPESGPGTSTEPSEGSAPGSPAGSPTSTE | 318 |
| | EGTSTEPSEGSAPGTSTEPSEGSAPGTSESATPESGPGSEPATSGSETPGSE | |
| | PATSGSETPGSPAGSPTSTEEGTSESATPESGPGTESASR | |
| AE288_R1 | SAGSPTGPGSEPATSGSETPGTSESATPESGPGSEPATSGSETPGTSESAT | 319 |
| | PESGPGTSTEPSEGSAPGSPAGSPTSTEEGTSESATPESGPGSEPATSGSET | |
| | PGTSESATPESGPGSPAGSPTSTEEGSPAGSPTSTEEGTSTEPSEGSAPGTS | |
| | ESATPESGPGTSESATPESGPGTSESATPESGPGSEPATSGSETPGSEPATS | |
| | GSETPGSPAGSPTSTEEGTSTEPSEGSAPGTSTEPSEGSAPGSEPATSGSET | |
| | PGTSESATPESGPGTSTEPSEGSAPSASR | |
| AE432_R1 | SAGSPGSPAGSPTSTEEGTSESATPESGPGTSTEPSEGSAPGSPAGSPTSTE | 320 |
| | EGTSTEPSEGSAPGTSTEPSEGSAPGTSESATPESGPGSEPATSGSETPGSE | |
| | PATSGSETPGSPAGSPTSTEEGTSESATPESGPGTSTEPSEGSAPGTSTEPS | |
| | EGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPG | |
| | TSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEG | |
| | SAPGTSTEPSEGSAPGTSTEPSEGSAPGTSTEPSEGSAPGTSTEPSEGSAP | |
| | GSPAGSPTSTEEGTESASR | |
| AE576_R1 | SAGSPTEEGTSESATPESGPGSEPATSGSETPGTSESATPESGPGTSTEPSE | 321 |
| | GSAPGTSTEPSEGSAPGTSTEPSEGSAPGTSTEPSEGSAPGTSTEPSEGSA | |
| | PGTSTEPSEGSAPGSPAGSPTSTEEGTSTEPSEGSAPGTSESATPESGPGSE | |
| | PATSGSETPGTSESATPESGPGSEPATSGSETPGTSESATPESGPGTSTEPS | |
| | EGSAPGTSESATPESGPGSPAGSPTSTEEGSPAGSPTSTEEGSPAGSPTST | |
| | EEGTSESATPESGPGTSTEPSEGSAPGTSESATPESGPGSEPATSGSETPG | |
| | TSESATPESGPGSEPATSGSETPGTSESATPESGPGTSTEPSEGSAPGSPA | |
| | GSPTSTEEGTSESATPESGPGSEPATSGSETPGTSESATPESGPGSPAGSPT | |
| | STEEGSPAGSPTSTEEGTSTEPSEGSAPGTSESATPESGPGTSESATPESGP | |
| | GTSESATPESGPGSEPATSGSETPGSEPATSGSETPGSPAGSPTSTEEGTST | |
| | EPSEGSAPGTSTEPSEGSAPGSEPATSGSETPGTSESATPESGPGTSTEPSE | |
| | GSAPSASR | |
| AE864_R1 | SAGSPGSPAGSPTSTEEGTSESATPESGPGTSTEPSEGSAPGSPAGSPTSTE | 322 |
| | EGTSTEPSEGSAPGTSTEPSEGSAPGTSESATPESGPGSEPATSGSETPGSE | |
| | PATSGSETPGSPAGSPTSTEEGTSESATPESGPGTSTEPSEGSAPGTSTEPS | |
| | EGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPG | |
| | TSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEG | |
| | SAPGTSTEPSEGSAPGTSTEPSEGSAPGTSTEPSEGSAPGTSTEPSEGSAP | |
| | GSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATP | |
| | ESGPGSPAGSPTSTEEGSPAGSPTSTEEGSPAGSPTSTEEGTSESATPESGP | |
| | GTSTEPSEGSAPGTSESATPESGPGSEPATSGSETPGTSESATPESGPGSEP | |
| | ATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEE | |
| | GTSTEPSEGSAPGTSESATPESGPGTSESATPESGPGTSESATPESGPGSEP | |
| | ATSGSETPGSEPATSGSETPGSPAGSPTSTEEGTSTEPSEGSAPGTSTEPSE | |
| | GSAPGSEPATSGSETPGTSESATPESGPGTESASR | |
| AF864_R1 | SAGSPGSTSSTAESPGPGSTSSTAESPGPGSTSESPSGTAPGSTSSTAESPG | 323 |
| | PGSTSSTAESPGPGTSTPESGSASPGSTSESPSGTAPGTSPSGESSTAPGST | |
| | SESPSGTAPGSTSESPSGTAPGTSPSGESSTAPGSTSESPSGTAPGSTSESP | |
| | SGTAPGTSPSGESSTAPGSTSESPSGTAPGSTSESPSGTAPGSTSESPSGTA | |
| | PGTSTPESGSASPGSTSESPSGTAPGTSTPESGSASPGSTSSTAESPGPGST | |
| | SSTAESPGPGTSTPESGSASPGTSTPESGSASPGSTSESPSGTAPGTSTPES | |
| | GSASPGTSTPESGSASPGSTSESPSGTAPGSTSESPSGTAPGSTSESPSGTA | |
| | PGSTSSTAESPGPGTSTPESGSASPGTSTPESGSASPGSTSESPSGTAPGST | |
| | SESPSGTAPGTSTPESGSASPGSTSESPSGTAPGSTSESPSGTAPGTSTPES | |
| | GSASPGTSPSGESSTAPGSTSSTAESPGPGTSPSGESSTAPGSTSSTAESPG | |
| | PGTSTPESGSASPGSTSESPSGTAPGSTSSTAESPGPGTSTPESGSASPGTS | |
| | TPESGSASPGSTSSTAESPGPGTSPSGESSTAPGTSTPESGSASPGSTSESP | |
| | SGTAPGSTSESPSGTAPGTSTPESGSASPGTSPSGESSTAPGSTSSTAESPG | |
| | PGTSPSGESSTAPGSTSSTAESPGPGSTSSTAESPGPGTSPSGESSTAPGST | |
| | SESPSGTAPGSTSESPSGTAPGTSTPESGSASPGSTSESPSGTAPGSTSESP | |
| | SGTAPGTSTPESGSASPGSTSESPSGTAPGSTSESPSGTAPGSTSESPSGTA | |
| | PGSTSSTAESPGPGTSPSGESSTAPGTSSASR | |
| AG864_R1 | SAGSPGASPGTSSTGSPGSSPSASTGTGPGSSPSASTGTGPGTPGSGTASS | 324 |
| | SPGSSTPSGATGSPGSSPSASTGTGPGASPGTSSTGSPGTPGSGTASSSPG | |
| | SSTPSGATGSPGTPGSGTASSSPGASPGTSSTGSPGASPGTSSTGSPGTPG | |
| | SGTASSSPGSSTPSGATGSPGASPGTSSTGSPGTPGSGTASSSPGSSTPSG | |
| | ATGSPGSSPSASTGTGPGSSPSASTGTGPGSSTPSGATGSPGSSTPSGATG | |
| | SPGASPGTSSTGSPGASPGTSSTGSPGASPGTSSTGSPGTPGSGTASSSPG | |
| | ASPGTSSTGSPGASPGTSSTGSPGASPGTSSTGSPGSSPSASTGTGPGTPG | |
| | SGTASSSPGASPGTSSTGSPGASPGTSSTGSPGASPGTSSTGSPGSSTPSG | |
| | ATGSPGSSTPSGATGSPGASPGTSSTGSPGTPGSGTASSSPGSSTPSGATG | |
| | SPGSSTPSGATGSPGSSTPSGATGSPGSSPSASTGTGPGASPGTSSTGSPG | |
| | ASPGTSSTGSPGTPGSGTASSSPGASPGTSSTGSPGASPGTSSTGSPGASP | |
| | GTSSTGSPGASPGTSSTGSPGTPGSGTASSSPGSSTPSGATGSPGTPGSGT | |
| | ASSSPGSSTPSGATGSPGTPGSGTASSSPGSSTPSGATGSPGSSTPSGATG | |
| | SPGSSPSASTGTGPGSSPSASTGTGPGASPGTSSTGSPGTPGSGTASSSPG | |
| | SSTPSGATGSPGSSPSASTGTGPGSSPSASTGTGPGASPGTSSTGSPGASP | |
| | GTSSTGSPGSSTPSGATGSPGSSPSASTGTGPGASPGTSSTGSPGSSPSAST | |
| | GTGPGTPGSGTASSSPGSSTPSGATGSPGSSTPSGATGSPGASSASR | |
| AE864_R2 | GSPGAGSPAGSPTSTEEGTSESATPESGPGTSTEPSEGSAPGSPAGSPTST | 325 |
| | EEGTSTEPSEGSAPGTSTEPSEGSAPGTSESATPESGPGSEPATSGSETPGS | |
| | EPATSGSETPGSPAGSPTSTEEGTSESATPESGPGTSTEPSEGSAPGTSTEP | |
| | SEGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPG | |
| | TSTEPSEGSAPGTSESATPESGPGTSESATPESGPGSPAGSPTSTEEGTSES | |
| | ATPESGPGSEPATSGSETPGTSESATPESGPGTSTEPSEGSAPGTSTEPSEG | |
| | SAPGTSTEPSEGSAPGTSTEPSEGSAPGTSTEPSEGSAPGTSTEPSEGSAP | |
| | GSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATP | |
| | ESGPGSPAGSPTSTEEGSPAGSPTSTEEGSPAGSPTSTEEGTSESATPESGP | |
| | GTSTEPSEGSAPGTSESATPESGPGSEPATSGSETPGTSESATPESGPGSEP | |
| | ATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEE | |
| | GTSTEPSEGSAPGTSESATPESGPGTSESATPESGPGTSESATPESGPGSEP | |
| | ATSGSETPGSEPATSGSETPGSPAGSPTSTEEGTSTEPSEGSAPGTSTEPSE | |
| | GSAPGSEPATSGSETPGTSESATPESGPGTESASR | |
| AE713 | PGSPAGSPTSTEEGTSESATPESGPGTSTEPSEGSAPGSPAGSPTSTEEGTS | 326 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPA | |
| AE717 | PGSPAGSPTSTEEGTSESATPESGPGTSTEPSEGSAPGSPAGSPTSTEEGTS | 327 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPASASR | |
| AE146 | PGSPAGSPTSTEEGTSESATPESGPGTSTEPSEGSAPGSPAGSPTSTEEGTS | 328 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPG | |
| AE150 | PGSPAGSPTSTEEGTSESATPESGPGTSTEPSEGSAPGSPAGSPTSTEEGTS | 329 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGSASR | |
| AE818 | PGSPAGSPTSTEEGTSESATPESGPGTSTEPSEGSAPGSPAGSPTSTEEGTS | 330 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPG | |
| AE822 | PGSPAGSPTSTEEGTSESATPESGPGTSTEPSEGSAPGSPAGSPTSTEEGTS | 331 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPGSASR | |
| AE626 | PGSPAGSPTSTEEGTSESATPESGPGTSTEPSEGSAPGSPAGSPTSTEEGTS | 332 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPG | |
| AE630 | PGSPAGSPTSTEEGTSESATPESGPGTSTEPSEGSAPGSPAGSPTSTEEGTS | 333 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGSASR | |
| AE1298 | PGSPAGSPTSTEEGTSESATPESGPGTSTEPSEGSAPGSPAGSPTSTEEGTS | 334 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPGTSTEPSEGSA | |
| | PGSEPATSGSETPGTSESATPESGPGTSTEPSEGSAPGSPAGSPTSTEEGTS | |
| | ESATPESGPGTSTEPSEGSAPGSPAGSPTSTEEGTSTEPSEGSAPGTSTEPS | |
| | EGSAPGTSESATPESGPGSEPATSGSETPGSEPATSGSETPGSPAGSPTST | |
| | EEGTSESATPESGPGTSTEPSEGSAPGTSTEPSEGSAPGSPAGSPTSTEEG | |
| | TSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPE | |
| | SGPGTSESATPESGPGSPAGSPTSTEEGTSESATPESGPGSEPATSGSETP | |
| | GTSESATPESGPGTSTEPSEGSAPGTSTEPSEGSAPGTSTEPSEGSAPGTST | |
| | EPSEGSAPGTSTEPSEGSAPGTSTEPSEGSAPGSPAGSPTSTEEGTSTEPSE | |
| | GSAPG | |
| AE1302 | PGSPAGSPTSTEEGTSESATPESGPGTSTEPSEGSAPGSPAGSPTSTEEGTS | 335 |
| | TEPSEGSAPGTSTEPSEGSAPGTSESATPESGPGSEPATSGSETPGSEPATS | |
| | GSETPGSPAGSPTSTEEGTSESATPESGPGTSTEPSEGSAPGTSTEPSEGSA | |
| | PGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPS | |
| | EGSAPGTSESATPESGPGTSESATPESGPGSPAGSPTSTEEGTSESATPES | |
| | GPGSEPATSGSETPGTSESATPESGPGTSTEPSEGSAPGTSTEPSEGSAPG | |
| | TSTEPSEGSAPGTSTEPSEGSAPGTSTEPSEGSAPGTSTEPSEGSAPGSPA | |
| | GSPTSTEEGTSTEPSEGSAPGTSESATPESGPGSEPATSGSETPGTSESATP | |
| | ESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTST | |
| | EPSEGSAPGTSESATPESGPGSEPATSGSETPGTSESATPESGPGSEPATS | |
| | GSETPGTSESATPESGPGTSTEPSEGSAPGSPAGSPTSTEEGTSESATPESG | |
| | PGSEPATSGSETPGTSESATPESGPGSPAGSPTSTEEGSPAGSPTSTEEGTS | |
| | TEPSEGSAPGTSESATPESGPGTSESATPESGPGTSESATPESGPGSEPATS | |
| | GSETPGSEPATSGSETPGSPAGSPTSTEEGTSTEPSEGSAPGTSTEPSEGSA | |
| | PGSEPATSGSETPGTSESATPESGPGTSTEPSEGSAPGSPAGSPTSTEEGTS | |
| | ESATPESGPGTSTEPSEGSAPGSPAGSPTSTEEGTSTEPSEGSAPGTSTEPS | |
| | EGSAPGTSESATPESGPGSEPATSGSETPGSEPATSGSETPGSPAGSPTST | |
| | EEGTSESATPESGPGTSTEPSEGSAPGTSTEPSEGSAPGSPAGSPTSTEEG | |
| | TSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPE | |
| | SGPGTSESATPESGPGSPAGSPTSTEEGTSESATPESGPGSEPATSGSETP | |
| | GTSESATPESGPGTSTEPSEGSAPGTSTEPSEGSAPGTSTEPSEGSAPGTST | |
| | EPSEGSAPGTSTEPSEGSAPGTSTEPSEGSAPGSPAGSPTSTEEGTSTEPSE | |
| | GSAPGSASR | |
| AF713 | GSTSSTAESPGPGSTSSTAESPGPGSTSESPSGTAPGSTSSTAESPGPGSTS | 336 |
| | STAESPGPGTSTPESGSASPGSTSESPSGTAPGTSPSGESSTAPGSTSESPS | |
| | GTAPGSTSESPSGTAPGTSPSGESSTAPGSTSESPSGTAPGSTSESPSGTAP | |
| | GTSPSGESSTAPGSTSESPSGTAPGSTSESPSGTAPGSTSESPSGTAPGTST | |
| | PESGSASPGSTSESPSGTAPGTSTPESGSASPGSTSSTAESPGPGSTSSTAE | |
| | SPGPGTSTPESGSASPGTSTPESGSASPGSTSESPSGTAPGTSTPESGSASP | |
| | GTSTPESGSASPGSTSESPSGTAPGSTSESPSGTAPGSTSESPSGTAPGSTS | |
| | STAESPGPGTSTPESGSASPGTSTPESGSASPGSTSESPSGTAPGSTSESPS | |
| | GTAPGTSTPESGSASPGSTSESPSGTAPGSTSESPSGTAPGTSTPESGSASP | |
| | GTSPSGESSTAPGSTSSTAESPGPGTSPSGESSTAPGSTSSTAESPGPGTST | |
| | PESGSASPGSTSESPSGTAPGSTSSTAESPGPGTSTPESGSASPGTSTPESG | |
| | SASPGSTSSTAESPGPGTSPSGESSTAPGTSTPESGSASPGSTSESPSGTAP | |
| | GSTSESPSGTAPGTSTPESGSASPGTSPSGESSTAPGSTSSTAESPGPGTSP | |
| | SGESSTAPGSTSSTAESPGPGSTSSTAESPGPGTSPS | |
| AF717 | GSTSSTAESPGPGSTSSTAESPGPGSTSESPSGTAPGSTSSTAESPGPGSTS | 337 |
| | STAESPGPGTSTPESGSASPGSTSESPSGTAPGTSPSGESSTAPGSTSESPS | |
| | GTAPGSTSESPSGTAPGTSPSGESSTAPGSTSESPSGTAPGSTSESPSGTAP | |
| | GTSPSGESSTAPGSTSESPSGTAPGSTSESPSGTAPGSTSESPSGTAPGTST | |
| | PESGSASPGSTSESPSGTAPGTSTPESGSASPGSTSSTAESPGPGSTSSTAE | |
| | SPGPGTSTPESGSASPGTSTPESGSASPGSTSESPSGTAPGTSTPESGSASP | |
| | GTSTPESGSASPGSTSESPSGTAPGSTSESPSGTAPGSTSESPSGTAPGSTS | |
| | STAESPGPGTSTPESGSASPGTSTPESGSASPGSTSESPSGTAPGSTSESPS | |
| | GTAPGTSTPESGSASPGSTSESPSGTAPGSTSESPSGTAPGTSTPESGSASP | |
| | GTSPSGESSTAPGSTSSTAESPGPGTSPSGESSTAPGSTSSTAESPGPGTST | |
| | PESGSASPGSTSESPSGTAPGSTSSTAESPGPGTSTPESGSASPGTSTPESG | |
| | SASPGSTSSTAESPGPGTSPSGESSTAPGTSTPESGSASPGSTSESPSGTAP | |
| | GSTSESPSGTAPGTSTPESGSASPGTSPSGESSTAPGSTSSTAESPGPGTSP | |
| | SGESSTAPGSTSSTAESPGPGSTSSTAESPGPGTSPSSASR | |
| AG713 | GASPGTSSTGSPGSSPSASTGTGPGSSPSASTGTGPGTPGSGTASSSPGSS | 338 |
| | TPSGATGSPGSSPSASTGTGPGASPGTSSTGSPGTPGSGTASSSPGSSTPS | |
| | GATGSPGTPGSGTASSSPGASPGTSSTGSPGASPGTSSTGSPGTPGSGTAS | |
| | SSPGSSTPSGATGSPGASPGTSSTGSPGTPGSGTASSSPGSSTPSGATGSP | |
| | GSSPSASTGTGPGSSPSASTGTGPGSSTPSGATGSPGSSTPSGATGSPGAS | |
| | PGTSSTGSPGASPGTSSTGSPGASPGTSSTGSPGTPGSGTASSSPGASPGT | |
| | SSTGSPGASPGTSSTGSPGASPGTSSTGSPGSSPSASTGTGPGTPGSGTAS | |
| | SSPGASPGTSSTGSPGASPGTSSTGSPGASPGTSSTGSPGSSTPSGATGSP | |
| | GSSTPSGATGSPGASPGTSSTGSPGTPGSGTASSSPGSSTPSGATGSPGSS | |
| | TPSGATGSPGSSTPSGATGSPGSSPSASTGTGPGASPGTSSTGSPGASPGT | |
| | SSTGSPGTPGSGTASSSPGASPGTSSTGSPGASPGTSSTGSPGASPGTSST | |
| | GSPGASPGTSSTGSPGTPGSGTASSSPGSSTPSGATGSPGTPGSGTASSSP | |
| | GSSTPSGATGSPGTPGSGTASSSPGSSTPSGATGSPGSSTPSGATGSPGSS | |
| | PSASTGTGPGSSPSASTGTGPGASPGTSSTGSPGTPGSGTASSSPGSSTP | |
| AG717 | GASPGTSSTGSPGSSPSASTGTGPGSSPSASTGTGPGTPGSGTASSSPGSS | 339 |
| | TPSGATGSPGSSPSASTGTGPGASPGTSSTGSPGTPGSGTASSSPGSSTPS | |
| | GATGSPGTPGSGTASSSPGASPGTSSTGSPGASPGTSSTGSPGTPGSGTAS | |
| | SSPGSSTPSGATGSPGASPGTSSTGSPGTPGSGTASSSPGSSTPSGATGSP | |
| | GSSPSASTGTGPGSSPSASTGTGPGSSTPSGATGSPGSSTPSGATGSPGAS | |
| | PGTSSTGSPGASPGTSSTGSPGASPGTSSTGSPGTPGSGTASSSPGASPGT | |
| | SSTGSPGASPGTSSTGSPGASPGTSSTGSPGSSPSASTGTGPGTPGSGTAS | |
| | SSPGASPGTSSTGSPGASPGTSSTGSPGASPGTSSTGSPGSSTPSGATGSP | |
| | GSSTPSGATGSPGASPGTSSTGSPGTPGSGTASSSPGSSTPSGATGSPGSS | |
| | TPSGATGSPGSSTPSGATGSPGSSPSASTGTGPGASPGTSSTGSPGASPGT | |
| | SSTGSPGTPGSGTASSSPGASPGTSSTGSPGASPGTSSTGSPGASPGTSST | |
| | GSPGASPGTSSTGSPGTPGSGTASSSPGSSTPSGATGSPGTPGSGTASSSP | |
| | GSSTPSGATGSPGTPGSGTASSSPGSSTPSGATGSPGSSTPSGATGSPGSS | |
| | PSASTGTGPGSSPSASTGTGPGASPGTSSTGSPGTPGSGTASSSPGSSTPS | |
| | ASR | |

In some embodiments wherein the XTEN has less than 100% of its amino acids consisting of 4, 5, or 6 types of amino acid selected from glycine (G), alanine (A), serine (S), threonine (T), glutamate (E) and proline (P), or less than 100% of the sequence consisting of the sequence motifs from Table 9 or the XTEN sequences of Table 10 and Table 11, the other amino acid residues of the XTEN are selected from any of the other 14 natural L-amino acids, but are preferentially selected from hydrophilic amino acids such that the XTEN sequence contains at least about 90%, 91%, 92%, 93%, 94%, 95%, 96%, 97%, 98%, or at least about 99% hydrophilic amino acids. An individual amino acid or a short sequence of amino acids other than glycine (G), alanine (A), serine (S), threonine (T), glutamate (E) and proline (P) may be incorporated into the XTEN to achieve a needed property, such as to permit incorporation of a restriction site by the encoding nucleotides, or to facilitate linking to a payload component by inclusion of cysteine or lysine amino acids, or incorporation of a cleavage sequence. As one exemplary embodiment, described more fully below, the XTEN incorporates from 1 to about 20, or 1 to about 15, or 1 to about 10, or 1 to about 5, or 9, or 3, or 2 cysteine residues, or a single cysteine residue wherein the reactive cysteines are utilized for linking to cross-linkers or targeting moieties or other XTEN, as described herein. In these embodiments, the incorporation of the lysine and/or cysteine residues does not otherwise affect the underlying properties of the XTEN, described herein. Specific embodiments of the foregoing XTEN with lysine and/or cyteine residues are set forth in Table 11. The XTEN amino acids that are not glycine (G), alanine (A), serine (S), threonine (T), glutamate (E) and proline (P) are either interspersed throughout the XTEN sequence, are located within or between the sequence motifs, or are concentrated in one or more short stretches of the XTEN sequence such as at or near the N- or C-terminus. As hydrophobic amino acids impart structure to a polypeptide, the invention provides that the content of hydrophobic amino acids in the XTEN utilized in the conjugation constructs will typically be less than 5%, or less than 2%, or less than 1% of the total amino acids incorporated into the XTEN. Hydrophobic residues that are less favored in construction of XTEN include tryptophan, phenylalanine, tyrosine, leucine, isoleucine, valine, and methionine. Additionally, one can design the XTEN sequences to contain less than 5% or less than 4% or less than 3% or less than 2% or less than 1% or none of the following amino acids: methionine (to avoid oxidation), asparagine and glutamine (to avoid desamidation). In other embodiments, the amino acid content of methionine and tryptophan in the XTEN component used in the conjugation constructs is typically less than 5%, or less than 2%, and most preferably less than 1%. In other embodiments, the XTEN of the subject XTEN conjugates will have a sequence that has less than 10% amino acid residues with a positive charge, or less than about 7%, or less that about 5%, or less than about 2% amino acid residues with a positive charge, the sum of methionine and tryptophan residues will be less than 2%, and the sum of asparagine and glutamine residues will be less than 5% of the total XTEN sequence.

### 3. Cysteine- and Lysine-Engineered XTEN Sequences

In another aspect, the invention provides XTEN for incorporation into the subject composition that have defined numbers of incorporated cysteine or lysine residues; “cysteine-engineered XTEN” and “lysine-engineered XTEN”, respectively. It is an object of the invention to provide XTEN with defned numbers of cysteine and/or lysine residues to permit conjugation between the thiol group of the cysteine or the epsilon amino group of the lysine and a reactive group on a payload, targeting moiety, or a cross-linker to be conjugated to the engineered XTEN. In one embodiment of the foregoing, the lysine-engineered XTEN of the invention has a single lysine residue, preferentially located at or near the C-terminus of the XTEN. In another embodiment of the foregoing, the cysteine-engineered XTEN of the invention has between 1 to about 20 cysteine residues, or about 1 to about 10 cysteine residues, or about 1 to about 5 cysteine residues, or 1 to about 3 cysteine residues, or 9 cysteine residues, or 3 cysteine residues, or 2 cysteine residues, or alternatively only a single cysteine residue. Using the foregoing lysine- and/or cysteine-containing XTEN embodiments, conjugates can be constructed that comprise a payload, a targeting moiety, one or more XTEN (which may have a linked cross-linker or payload or targeting moiety) used to create the subject targeted conjugate compositions that are useful in the treatment of a disease in a subject. In one embodiment, the cysteine-engineered XTEN would serve as a backbone carrier to which individual targeted conjugate fusion proteins could be linked using PCM such that the linked individual targeted conjugate fusion proteins would be released when in proximity to a target tissue colocalized with a protease capable of cleaving the PCM. In another embodiment, the cysteine-engineered XTEN are used to make configurations bearing 2, 3, 4 or more XTEN linked to a common cross-linker resulting in multivalent constructs in order to increase the overall molecular weight and size of the targeted conjugate compositions. It will be understood that in the subject targeted conjugate compostions, the maximum number of molecules of the payload, targeting moiety or another XTEN linked to the engineered XTEN component is determined by the numbers of lysines, cysteines or other amino acids with a reactive side group (e.g., a terminal amino or thiol) incorporated into the XTEN.

In one embodiment, the invention provides cysteine-engineered XTEN where nucleotides encoding one or more amino acids of an XTEN (e.g., the XTEN of Table 10) are replaced with a cysteine amino acid to create the cysteine-engineered XTEN gene. In another embodiment, the invention provides cysteine-engineered XTEN where nucleotides encoding one or more cysteine amino acids are inserted into an-XTEN encoding gene to create the cysteine-engineered XTEN gene. In other cases, oligonucleotides encoding one or more motifs of about 9 to about 14 amino acids comprising codons encoding one or more cysteines are linked in frame with other oligos encoding XTEN motifs or full-length XTEN to create the cysteine-engineered XTEN gene. In one embodiment of the foregoing, where the one or more cysteines are inserted into an XTEN sequence during the creation of the XTEN gene, nucleotides encoding cysteine can be linked to codons encoding amino acids used in XTEN to create a cysteine-XTEN motif with the cysteine(s) at a defined position using the methods described herein, or by standard molecular biology techniques, and the motifs subsequently assembled into the gene encoding the full-length cysteine-engineered XTEN. In such cases, where, for example, nucleotides encoding a single cysteine are added to the DNA encoding a motif selected from Table 9, the resulting motif would have 13 amino acids, while incorporating two cysteines would result in a motif having 14 amino acids, etc. In other cases, a cysteine-motif can be created de novo and be of a pre-defined length and number of cysteine amino acids by linking nucleotides encoding cysteine to nucleotides encoding one or more amino acid residues used in XTEN (e.g., G, S, T, E, P, A) at a defined position, and the encoding motifs subsequently assembled by annealing with other XTEN-encoding motif sequences into the gene encoding the full-length XTEN, as described herein. In cases where a lysine-engineered XTEN is utilized to make the conjugates of the invention, the approaches described above would be performed with codons encoding lysine instead of cysteine. Thus, by the foregoing, a new XTEN motif can be created that could comprise about 9-14 amino acid residues and have one or more reactive amino acids; i.e., cysteine or lysine. Non-limiting examples of motifs suitable for use in an engineered XTEN that contain a single cysteine or lysine are:

(SEQ ID NO: 340)
GGSPAGSCTSP
(SEQ ID NO: 341)
GASASCAPSTG
(SEQ ID NO: 342)
TAEAAGCGTAEAA
(SEQ ID NO: 343)
GPEPTCPAPSG
(SEQ ID NO: 344)
GGSPAGSKTSP
(SEQ ID NO: 345)
GASASKAPSTG

However, the invention contemplates motifs of different lengths for incorporation into XTEN.

In one embodiment, the disclosure provides XTEN sequences with a single C-terminal lysine for linking to a payload, targeting moiety, or another XTEN. In another embodiment, the disclosure provides XTEN with 1 to 9 residues of cysteine wherein the sequences with multiple cyteine are interspersed across the length of the XTEN. In such cases where a gene encoding an XTEN with one or more cysteine or lysine residues is to be constructed from existing XTEN motifs or segments, the gene can be designed and built by linking existing “building block” polynucleotides encoding both short- and long-length XTENs; e.g., AE36, AE48, AE144, AE288, AE432, AE576, AE864, AM48, AM875, AE912, AG864, which can be fused in frame with the nucleotides encoding the cysteine- and/or lysine-containing motifs or, alternatively, the cysteine- and/or lysine-encoding nucelotides can be PCR'ed into an existing gene encoding an XTEN sequence using conventional PCR methods, or as described herein. For example, where an existing full-length XTEN gene is to be modified with nucleotides encoding one or more reactive cysteine or lysine residues, an oligonucleotide can be created that encodes a cysteine or lysine and that exhibits partial homology to and can hybridize with one or more short sequences of the XTEN, resulting in a recombination event and substitution of a cysteine or the lysine codon for an existing codon of the XTEN gene.The cysteine- or lysine-encoding oligonucleotides can be designed to hybridize with a given sequence segment at different points along the known XTEN sequence to permit their insertion into an XTEN-encoding gene. Thus, the invention contemplates that multiple XTEN gene constructs can be created with cysteines or lysines inserted at different locations within the XTEN sequence by the selection of restriction sites within the XTEN sequence and the design of oligonucleotides appropriate for the given location and that encode a cysteine or lysine, including use of designed oligonucleotides that result in multiple insertions in the same XTEN sequence. By the design and selection of one or more such oligonucleotides in consideration of the known sequence of the XTEN, and the appropriate use of the methods of the invention, the potential number of substituted reactive cysteine or lysine residues inserted into the full-length XTEN can be estimated and then confirmed by sequencing the resulting XTEN gene.

Non-limiting examples of cysteine- and lysine- engineered XTEN are provided in Table 11. Thus, in one embodiment, the invention provides an XTEN sequence having at least about 80% sequence identity, or at least about 90%, or about 91%, or about 92%, or about 93%, or about 94%, or about 95%, or about 96%, or about 97%, or about 98%, or about 99% sequence identity, or is identical to a sequence or a fragment of a sequence selected from of Table 11, when optimally aligned. However, application of the cysteine- or lysine-engineered methodology to create XTEN encompassing cysteine or lysine residues is not meant to be constrained to the precise compositions or range of composition identities of the foregoing embodiments. As will be appreciated by those skilled in the art, the precise location and numbers of incorporated cysteine or lysine residues in an XTEN can be varied without departing from the invention as described.

TABLE 11
Cysteine- and lysine-engineered XTEN

| | | SEQ |
|---|---|---|
| | | ID |
| Name | Ammo Acid Sequence | NO: |
| Seg | SAGSPTAEAAGCGTAEAAGSPAGSPTSTEEGTSESATPESGPGTSTEPSEGSAPG | 346 |
| 174 | SPAGSPTSTEEGTSTEPSEGSAPGTSTEPSEGSAPGTSESATPESGPGSEPATSGS | |
| | ETPGSEPATSGSETPGSPAGSPTSTEEGTSESATPESGPGTSTEPSEGSAPGTSTEP | |
| | SEGSAPGSPAGSPTSTEEGTSTEPSEGSAPGTSTEPSEGSAPGTSESATPESGPGT | |
| | STEPSEGSAPGTSESATPESGPGSEPATSGSETPGTSTEPSEGSAPGTSTEPSEGS | |
| | APGTSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSAPGT | |
| | STEPSEGSAPGTSTEPSEGSAPGTSTEPSEGSAPGSPAGSPTSTEEGTSTEPSEGS | |
| | APTAEAAGCGTAEAAGTSESATPESGPGSEPATSGSETPGTSESATPESGPGSEP | |
| | ATSGSETPGTSESATPESGPGTSTEPSEGSAPGTSESATPESGPGSPAGSPTSTEE | |
| | GSPAGSPTSTEEGSPAGSPTSTEEGTSESATPESGPGTSTEPSEGSAPGTSESATP | |
| | ESGPGSEPATSGSETPGTSESATPESGPGSEPATSGSETPGTSESATPESGPGTST | |
| | EPSEGSAPGSPAGSPTSTEEGTSESATPESGPGSEPATSGSETPGTSESATPESGP | |
| | GSPAGSPTSTEEGSPAGSPTSTEEGTSTEPSEGSAPGTSESATPESGPGTSESATP | |
| | ESGPGTSESATPESGPGSEPATSGSETPGSEPATSGSETPGSPAGSPTSTEEGTST | |
| | EPSEGSAPGTSTEPSEGSAPGSEPATSGSETPGTSESATPESGPGTSTEPSEGSAP | |
| | TAEAAGCGTAEAASASR | |
| Seg | SAGSPGSPAGSPTSTEEGTSESATPESGPGTSTEPSEGSAPGSPAGSPTSTEEGTS | 347 |
| 175 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPTAEAAGCGTA | |
| | EAAPGSEPATSGSETPGTSESATPESGPGSEPATSGSETPGTAEAAGCGTAEAAS | |
| | TEPSEGSAPGTSESATPESGPGSPAGSPTSTEEGSPATAEAAGCGTAEAASPTST | |
| | EEGTSESATPESGPGTSTEPSEGSAPGTSESATTAEAAGCGTAEAASETPGTSES | |
| | ATPESGPGSEPATSGSETPGTSESATPESGTAEAAGCGTAEAAGSPAGSPTSTEE | |
| | GTSESATPESGPGSEPATSGSETPGTTAEAAGCGTAEAAAGSPTSTEEGSPAGSP | |
| | TSTEEGTSTEPSEGSAPGTSESTAEAAGCGTAEAATPESGPGTSESATPESGPGS | |
| | EPATSGSETPGSEPATSGTAEAAGCGTAEAATEEGTSTEPSEGSAPGTSTEPSEG | |
| | SAPGSEPATSGSETPTAEAAGCGTAEAASASR | |
| Seg | SAGSPTAEAAGCGTAEAAGTSESATPESGPGSEPATSGSETPGTSESATPESGPG | 348 |
| 176 | TSTEPSEGSAPGTSESATPESGPGSPAGSPTSTEEGSPAGSPTSTEEGSPAGSPTST | |
| | EEGTSESATPESGPGTSTEPSEGSAPGTSESATPESGPGSEPATSGSETPGTSESA | |
| | TPESGPGSEPATSGSETPGTSESATPESGPGTSTEPSEGSAPTAEAAGCGTAEAA | |
| | GSPAGSPTSTEEGTSESATPESGPGSEPATSGSETPGTSESATPESGPGSPAGSPT | |
| | STEEGSPAGSPTSTEEGTSTEPSEGSAPGTSESATPESGPGTSESATPESGPGTSES | |
| | ATPESGPGSEPATSGSETPGSEPATSGSETPGSPAGSPTSTEEGTSTEPSEGSAPG | |
| | TSTEPSEGSAPGSEPATSGSETPTAEAAGCGTAEAASASR | |
| Seg | SAGSPTAEAAGCGTAEAAPGSEPATSGSETPGTSESATPESGPGSEPATSGSETP | 349 |
| 177 | GTAEAAGCGTAEAASTEPSEGSAPGTSESATPESGPGSPAGSPTSTEEGSPATAE | |
| | AAGCGTAEAASPTSTEEGTSESATPESGPGTSTEPSEGSAPGTSESATTAEAAGC | |
| | GTAEAASETPGTSESATPESGPGSEPATSGSETPGTSESATPESGTAEAAGCGTA | |
| | EAAGSPAGSPTSTEEGTSESATPESGPGSEPATSGSETPGTTAEAAGCGTAEAA | |
| | AGSPTSTEEGSPAGSPTSTEEGTSTEPSEGSAPGTSESTAEAAGCGTAEAATPES | |
| | GPGTSESATPESGPGSEPATSGSETPGSEPATSGTAEAAGCGTAEAATEEGTSTE | |
| | PSEGSAPGTSTEPSEGSAPGSEPATSGSETPTAEAAGCGTAEAASASR | |
| Seg | SAGSPTAEAAGCGTAEAAGSPAGSPTSTEEGTSESATPESGPGTSTEPSEGSAPG | 350 |
| 187 | SPAGSPTSTEEGTSTEPSEGSAPGTSTEPSEGSAPGTSESATPESGPGSEPATSGS | |
| | ETPGSEPATSGSETPGSPAGSPTSTEEGTSESATPESGPGTSTEPSEGSAPGTSTEP | |
| | SEGSAPGSPAGSPTSTEEGTSTEPSEGSAPGTSTEPSEGSAPGTSESATPESGPGT | |
| | STEPSEGSAPGTSESATPESGPGSEPATSGSETPGTSTEPSEGSAPGTSTEPSEGS | |
| | APGTSESATPESGPGTSESATPESGPGSPAGSPTSTEEGTSESATPESGPGSEPAT | |
| | SGSETPGTSESATPESGPGTSTEPSEGSAPGTSTEPSEGSAPGTSTEPSEGSAPGT | |
| | STEPSEGSAPGTSTEPSEGSAPGTSTEPSEGSAPGSPAGSPTSTEEGTSTEPSEGS | |
| | APGTSESATPESGPGSEPATSGSETPGTSESATPESGPGSEPATSGSETPGTSESA | |
| | TPESGPGTSTEPSEGSAPGTSESATPESGPGSPAGSPTSTEEGSPAGSPTSTEEGSP | |
| | AGSPTSTEEGTSESATPESGPGTSTEPSEGSAPGTSESATPESGPGSEPATSGSET | |
| | PGTSESATPESGPGSEPATSGSETPGTSESATPESGPGTSTEPSEGSAPGSPAGSP | |
| | TSTEEGTSESATPESGPGSEPATSGSETPGTSESATPESGPGSPAGSPTSTEEGSP | |
| | AGSPTSTEEGTSTEPSEGSAPGTSESATPESGPGTSESATPESGPGTSESATPESG | |
| | PGSEPATSGSETPGSEPATSGSETPGSPAGSPTSTEEGTSTEPSEGSAPGTSTEPSE | |
| | GSAPGSEPATSGSETPGTSESATPESGPGTSTEPSEGSAPSASR | |
| Seg | SAGSPTEGTSTEPSEGSAPGTSESTAEAAGCGTAEAATPESGPGTSESATPESGP | 351 |
| 188 | GSEPATSGSETPGSEPATSGTAEAAGCGTAEAATEEGTSTEPSEGSAPGTSTEPS | |
| | EGSAPGSEPATSGSETPTAEAAGCGTAEAASASR | |
| Seg | SAGSPTPGTSESATPESGPGSEPATSGSETPGTSESATPESGPGSEPATSGSETPG | 352 |
| 189 | TSESATPESGPGTSTEPSEGSAPGTSESATPESGPGSPAGSPTSTEEGSPAGSPTST | |
| | EEGSPAGSPTSTEEGTSESATPESGPGTSTEPSEGSAPGTSESATPESGPGSEPAT | |
| | SGSETPGTSESATPESGPGSEPATSGSETPGTSESATPESGPGTSTEPSEGSAPGSP | |
| | AGSPTSTEEGTSESATPESGPGSEPATSGSETPGTSESATPESGPGSPAGSPTSTE | |
| | EGSPAGSPTSTEEGTSTEPSEGSAPGTSESATPESGPGTSESATPESGPGTSESAT | |
| | PESGPGSEPATSGSETPGSEPATSGSETPGSPAGSPTSTEEGTSTEPSEGSAPGTS | |
| | TEPSEGSAPGSEPATSGSETPTAEAAGCGTAEAASASR | |
| Seg | SAGSPTGSEPATSGSETPGTSESATPESGPGSEPATSGSETPGTSESATPESGPGT | 353 |
| 190 | STEPSEGSAPGTSESATPESGPGSPAGSPTSTEEGSPAGSPTSTEEGSPAGSPTSTE | |
| | EGTSESATPESGPGTSTEPSEGSAPGTSESATPESGPGSEPATSGSETPGTSESAT | |
| | PESGPGSEPATSGSETPGTSESATPESGPGTSTEPSEGSAPTAEAAGCGTAEAAG | |
| | SPAGSPTSTEEGTSESATPESGPGSEPATSGSETPGTSESATPESGPGSPAGSPTST | |
| | EEGSPAGSPTSTEEGTSTEPSEGSAPGTSESTAEAAGCGTAEAATPESGPGTSES | |
| | ATPESGPGSEPATSGSETPGSEPATSGSETPGSPAGSPTSTEEGTSTEPSEGSAPG | |
| | TSTEPSEGSAPGSEPATSGSETPTAEAAGCGTAEAASASR | |
| Seg | SAGSPTPGTSESATPESGPGSEPATSGSETPGTSESATPESGPGSEPATSGSETPG | 354 |
| 191 | TSESATPESGPGTSTEPSEGSAPGTSESATPESGPGSPAGSPTSTEEGSPAGSPTST | |
| | EEGSPAGSPTSTEEGTSESATPESGPGTSTEPSEGSAPGTSESATPESGPGSEPAT | |
| | SGSETPGTSESATPESGPGSEPATSGSETPGTSESATPESGPGTSTEPSEGSAPGSP | |
| | AGSPTSTEEGTSESATPESGPGSEPATSGSETPGTSESATPESGPGSPAGSPTSTE | |
| | EGSPAGSPTSTEEGTSTEPSEGSAPGTSESTAEAAGCGTAEAATPESGPGTSESA | |
| | TPESGPGSEPATSGSETPGSEPATSGTAEAAGCGTAEAATEEGTSTEPSEGSAPG | |
| | TSTEPSEGSAPGSEPATSGSETPTAEAAGCGTAEAASASR | |
| Seg | SAGSPGSTSSTAESPGPGSTSSTAESPGPGCTSESPSGTAPGSTSSTAESPGPGSTS | 355 |
| 192 | STAESPGPGTSTPESGSASPGSTSCSPSGEAPGTSPSGESSTAPGSTSESPSGTAPG | |
| | STSESPSGTAPETSPSGESCTAPGSTSASR | |
| Seg | SAGSPGTPGSGTASSSPGSSTPSGATGSPGCAGSGTASSSPGSSTPSGATGSPGTP | 356 |
| 193 | GSGTASSSPGSSTPSGATGSPGSSTCSGATGSPGSSPSASTGTGPGSSPSASTGTG | |
| | PGASPGTSSTGSPGTPGSGTACSSPGSSSASR | |
| Seg | SAGSPGSPAGSPTSTEEGTSESATPESGPGTSTEPSEGSAPGSPAGSPTSTEEGTS | 357 |
| 194 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGTSESATPESGPGTESASK | |
| Seg | SAGSPTGPGSEPATSGSETPGTSESATPESGPGSEPATSGSETPGTSESATPESGP | 358 |
| 195 | GTSTEPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGPGTSE | |
| | SATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTACSE | |
| | GSAPSASR | |
| Seg | SAGSPTGPGSEPATSGSETPGTSESATPESGPGSEPATSGSETPGTSESATPESGP | 359 |
| 196 | GTSTEPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGPGTSE | |
| | SATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTEPSE | |
| | GSCASASR | |
| Seg | SAGSPGSCAGSPTSTEEGTSESACPESGPGTSTEPSEGSCPGSPAGSPTSTEEGTC | 360 |
| 197 | TEPSEGSAPGTSTEPCSGSAPGTSESATPESCPGSEPATSGSETPGSCPATSGSET | |
| | PGSPAGSCTSTEEGTSESATPESCPGTESASR | |
| Seg | SAGSPTGCGSEPATSGSETPGTSESATPESGPGSEPATSGSETPGTSESATPESGP | 361 |
| 198 | GTSTEPSEGSAPGSPAGSPTSTEEGTSESATPESGPGSEPATSGSETPGTSESATP | |
| | ESGPGSPAGSPTSTEEGSPAGSPTSTEEGTSCTPSEGSAPGTSESATPESGPGTSE | |
| | SATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTEPSC | |
| | GSAPSASR | |
| Seg | SAGSPTGCGSEPATSGSETPGTSESATPESGPGSEPATSGSCTPGTSESATPESGP | 362 |
| 199 | GTSTEPSEGSAPGSPAGSPCSTEEGTSESATPESGPGSEPATSGSETPGTSESCTP | |
| | ESGPGSPAGSPTSTEEGSPAGSPTSTEEGTSCTPSEGSAPGTSESATPESGPGTSE | |
| | SATPESGPGCSESATPESGPGSEPATSGSETPGSEPATSGSETCGSPAGSPTSTEE | |
| | GTSTEPSEGSAPGTSTEPSEGCAPGSEPATSGSETPGTSESATPESGPGTSTEPSC | |
| | GSAPSASR | |
| Seg | SAGSPTGSEPATSGSETPGTSESATPESGPGSEPATSGSETPGTSESATPESGPGT | 363 |
| 200 | STEPSEGSAPGTSESATPESGPGSPAGSPTSTEEGSPAGSPTSTEEGSPAGSPTSTE | |
| | EGTSESATPESGPGTSTEPSEGSAPGTSESATPESGPGSEPATSGSETPGTSESAT | |
| | PESGPGSEPATSGSETPGTSESATPESGPGTSTEPSEGSAPTAEAAGCGTAEAAG | |
| | SPAGSPTSTEEGTSESATPESGPGSEPATSGSETPGTSESATPESGPGSPAGSPTST | |
| | EEGSPAGSPTSTEEGTSTEPSEGSAPGTSESATPESGPGTSESATPESGPGTSESA | |
| | TPESGPGSEPATSGSETPGSEPATSGSETPGSPAGSPTSTEEGTSTEPSEGSAPGT | |
| | STEPSEGSAPGSEPATSGSETPGTSESATPESGPGSASR | |
| Seg | SAGSPGAPGTSESATPESGPGTSESATPESGPGTSESATPESGPGSEPATSGSETP | 364 |
| 201 | GSEPATSGSETPGSPAGSPTSTEEGTSTEPSEGSAPGTSTEPSEGSAPGSEPATSG | |
| | SETPGTSESATPESGPGTSTEPSEGSCASASR | |
| Seg | SAGSPTAEAAGCGTAEAATSESATPESGPGTSTEPSEGSAPGSPAGSPTSTEEGT | 365 |
| 203 | STEPSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSPAGS | |
| | PTSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGTSESATPESGPGTESASR | |
| Seg | SAGSPGSPAGSPTSTEEGTSESATPESGPGTSTEPSEGSAPGSPAGSPTSTEEGTS | 366 |
| 204 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGSTAEAAGCGTAEAASASR | |
| Seg | SAGSPTAEAAGCGTAEAATSESATPESGPGTSTEPSEGSAPGSPAGSPTSTEEGT | 367 |
| 205 | STEPSEGSAPGTSTEPSEGSAPGTSESATPESGPGSEPATSGSETPGSEPATSGSE | |
| | TPGSPAGSPTSTEEGTSESATPESGPGTSTEPSEGSAPGTSTEPSEGSAPGSPAGS | |
| | PTSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEETAEAAGCGTAEAATSESATPES | |
| | GPGSEPATSGSETPGTSESATPESGPGSEPATSGSETPGTSESATPESGPGTSTEP | |
| | SEGSAPGTSESATPESGPGSPAGSPTSTEEGSPAGSPTSTEEGSPAGSPTSTEEGT | |
| | SESATPESGPGTSTEPSEGSAPGTSESATPESGPGSEPATSGSETPGTSESATPES | |
| | GPGSEPATSGSETPGTSESATPESGPGTSTEPSEGSAPGSPAGSPTSTEEGTSESA | |
| | TPESGPGSEPATSGSETPGTSESATPESGPGSPAGSPTSTEEGSPAGSPTSTEEGT | |
| | STEPSEGSAPGTSESATPESGPGTSESATPESGPGTSESATPESGPGSEPATSGSE | |
| | TPGSEPATSGSETPGSPAGSPTSTEEGTSTEPSEGSAPGTSTEPSEGSAPGSEPAT | |
| | SGSETPGSTAEAAGCGTAEAASASR | |
| Seg | SAGSPTGSEPATSGSETPGTSESATPESGPGSEPATSGSETPGTSESATPESGPGT | 368 |
| 206 | STEPSEGSAPGTSESATPESGPGSPAGSPTSTEEGSPAGSPTSTEEGSPAGSPTSTE | |
| | EGTSESATPESGPGTSTEPSEGSAPGTSESATPESGPGSEPATSGSETPGTSESAT | |
| | PESGPGSEPATSGSETPGTSESATPESGPGTSTEPSEGSAPTAEAAGCGTAEAAG | |
| | SPAGSPTSTEEGTSESATPESGPGSEPATSGSETPGTSESATPESGPGSPAGSPTST | |
| | EEGSPAGSPTSTEEGTSTEPSEGSAPGTSESATPESGPGTSESATPESGPGTSESA | |
| | TPESGPGSEPATSGSETPGSEPATSGSETPGSPAGSPTSTEEGTSTEPSEGSAPGT | |
| | STEPSEGSAPGSEPATSGSETPTAEAAGCGTAEAASASR | |
| Seg | ATAEAAGCGTAEAAGSPAGSPTSTEEGTSESATPESGPGTSTEPSEGSAPGSPA | 369 |
| 207 | GSPTSTEEGTSTEPSEGSAPGTSTEPSEGSAPGTSESATPESGPGSEPATSGSETP | |
| | GSEPATSGSETPGSPAGSPTSTEEGTSESATPESGPGTSTEPSEGSAPGTSTEPSE | |
| | GSAPGSPAGSPTSTEEGTSTEPSEGSAPGTSTEPSEGSAPGTSESATPESGPGTST | |
| | EPSEGSAPGTSESATPESGPGSEPATSGSETPGTSTEPSEGSAPGTSTEPSEGSAP | |
| | GTSESATPESGPGTSESATPESGPGSPAGSPTSTEEGTSESATPESGPGSEPATSG | |
| | SETPGTSESATPESGPGTSTEPSEGSAPGTSTEPSEGSAPGTSTEPSEGSAPGTSTE | |
| | PSEGSAPGTSTEPSEGSAPGTSTEPSEGSAPGSPAGSPTSTEEGTSTEPSEGSAPG | |
| | TSESATPESGPGSEPATSGSETPGTSESATPESGPGSEPATSGSETPGTSESATPES | |
| | GPGTSTEPSEGSAPGTSESATPESGPGSPAGSPTSTEEGSPAGSPTSTEEGSPAGS | |
| | PTSTEEGTSESASASR | |
| Seg | SAGSPGSPAGSPTSTENLYFQSATPESGPGTSTEPSEGSAPGSPAGSPTSTEEGTS | 370 |
| 208 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGSTAEAAGCGTAEAASASR | |
| Seg | SACSPGSPAGSPTSTEEGTSESATPESGPGTSTEPSEGSAPGSPAGSPTSTEEGTS | 371 |
| 210 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGTSESATPESGPGTCSASR | |
| Seg | SAGSPGSPAGSPTSTEEGTSESATPESGPGTSTEPSEGSAPGSPAGSPTSTEEGTS | 372 |
| 211 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATCG | |
| | SETPGTSESATPESGPGTCSASR | |
| Seg | SAGSPGSCAGSPTSTEEGTSESACSPEGPGTSTEPSEGSCGPSPAGSPTSTEEGTC | 373 |
| 212 | TEPSEGSAPGTSEPTCSGSAPGTSESATPESCGPSEPATSGSETPGSCAPTSGSET | |
| | PGSPAGSCTSTEEGTSESATPESCGPTESASR | |
| Seg | SAGSPGSCSSTAESPGPGSTSSTCSEPGPGSTSESPSGTCGPSTSSTAESPGPGSCS | 374 |
| 213 | STAESPGPGTSPETCGSASPGSTSESPSGTCGPTSPSGESSTAPGSCSESPSGTAP | |
| | GSTSESCSGTAPETSPSGESSTCGPSTSASR | |
| Seg | SAGSPGSPAGSPTSTEEGTSESATPESGPGTSTEPSEGSAPGSPAGSPTSTEEGTS | 375 |
| 214 | TEPSEGSAPGTSTEPSEGSAPGTSESATPESGPGSEPATSGSETPGSEPATSGSET | |
| | PGSPAGSPTSTEEGTSESATPESGPGTESASK | |
| Seg | SAGSPTGPGSEPATSGSETPGTSESATPESGPGSEPATSGSETPGTSESATPESGP | 376 |
| 215 | GTSTEPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGPGTSE | |
| | SATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTEPSE | |
| | GSAPSASK | |
| Seg | SAGSPTGTSESATPESGPGTSTEPSEGSAPGTSESATPESGPGSEPATSGSETPGT | 377 |
| 216 | SESATPESGPGSEPATSGSETPGTSESATPESGPGTSTEPSEGSAPTAEAAGCGT | |
| | AEAAGSPAGSPTSTEEGTSESATPESGPGSEPATSGSETPGTSESATPESGPGSPA | |
| | GSPTSTEEGSPAGSPTSTEEGTSTEPSEGSAPGTSESATPESGPGSASR | |
| Seg | SAGSPTGSPAGSPTSTEEGSPAGSPTSTEEGSPAGSPTSTEEGTSESATPESGPGT | 378 |
| 217 | STEPSEGSAPGTSESATPESGPGSEPATSGSETPGTSESATPESGPGSEPATSGSE | |
| | TPGTSESATPESGPGTSTEPSEGSAPTAEAAGCGTAEAAGSPAGSPTSTEEGTSE | |
| | SATPESGPGSEPATSGSETPGTSESATPESGPGSPAGSPTSTEEGSPAGSPTSTEE | |
| | GTSTEPSEGSAPGTSESATPESGPGTSESATPESGPGTSESATPESGPGSEPATSG | |
| | SETPGSASR | |
| Seg | SAGSPGSCAGSPTSTENLYFQSATPESGPGTSTEPSEGSAPGSPAGSPTSTEEGTS | 379 |
| 218 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGTSESATPESGPGTESASPSAHHHHHHHH | |
| Seg | SAGSPGTSESATPESGPGSEPATSGSETPGTSTEPSEGSAPGTSTEPSEGSAPGTS | 380 |
| 219 | ESATPESGPGTSESATPESGPGSPAGSPTSTEEGTSESATPESGPGSEPATSGSET | |
| | PGTSESATPESGPGTSTEPSEGSAPGTSTEPSEGSAPGTSTEPSEGSAPGTSTEPSE | |
| | GSAPGTSTEPSEGSAPGTSTEPSEGSAPGSPAGSPTSTEEGTSTEPSEGSAPGTSE | |
| | SATPESGPGSEPATSGSETPGTSESATPESGPGSEPATSGSETPGTSESATPESGP | |
| | GTSTEPSEGSAPGTSESATPESGPGSPAGSPTSTEEGSPAGSPTSTEEGSPAGSPT | |
| | STEEGTSESATPESGPGTSTEPSEGSAPGTSESATPESGPGSEPATSGSETPGTSES | |
| | ATPESGPGSEPATSGSETPGTSESATPESGPGTSTEPSEGSAPGSPAGSPTSTEEG | |
| | TSESATPESGPGSEPATSGSETPGTSESATPESGPGSPAGSPTSTEEGSPAGSPTST | |
| | EEGTSTEPSEGSAPGTSESATPESGPGTSESATPESGPGTSESATPESGPGSEPAT | |
| | SGSETPGSEPATSGSETPGSPAGSPTSTEEGTSTEPSEGSAPGTSTEPSEGSAPGS | |
| | EPATSGSETPGSTAEAAGCGTAEAASASR | |
| Seg | GSPGAGSPAGSPTSTEEGTSESATPESGPGTSTEPSEGSAPGSPAGSPTSTEEGTS | 381 |
| 220 | TEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSP | |
| | TSTEEGTSTEPSEGSAPGTSTEPSEGSAPGTSESATPESGPGTSTEPSEGSAPGTS | |
| | ESATPESGPGSEPATSGSETPGTSTEPSEGSAPGTSTEPSEGSAPGTSESATPESG | |
| | PGTSESATPESGPGSPAGSPTSTEEGTSESATPESGPGSEPATSGSETPGTSESAT | |
| | PESGPGTSTEPSEGSAPGTSTEPSEGSAPGTSTEPSEGSAPGTSTEPSEGSAPGTS | |
| | TEPSEGSAPGTSTEPSEGSAPGSPAGSPTSTEEGTSTEPSEGSAPGTSESATPESG | |
| | PGSEPATSGSETPGTSESATPESGPGSEPATSGSETPGTSESATPESGPGTSTEPSE | |
| | GSAPGTSESATPESGPGSPAGSPTSTEEGSPAGSPTSTEEGSPAGSPTSTEEGTSE | |
| | SATPESGPGTSTEPSEGSAPGTSESATPESGPGSEPATSGSETPGTSESATPESGP | |
| | GSEPATSGSETPGTSESATPESGPGTSTEPSEGSAPGSPAGSPTSTEEGTSESATP | |
| | ESGPGSEPATSGSETPGTSESATPESGPGSPAGSPTSTEEGSPAGSPTSTEEGTST | |
| | EPSEGSAPGTSESATPESGPGTSESATPESGPGTSESATPESGPGSEPATSGSETP | |
| | GSEPATSGSETPGSPAGSPTSTEEGTSTEPSEGSAPGTSTEPSEGSAPGSEPATSG | |
| | SETPGTSESATPESGPGTESASK | |
| Seg | GSPGAGPSEPATSGSETPGTSESATPESGPGSEPATSGSETPGTSESATPESGPGT | 382 |
| 221 | STEPSEGSAPTAEAAGCGTAEAAGSPAGSPTSTEEGTSESATPESGPGSEPATSG | |
| | SETPGTSESATPESGPGSPAGSPTSTEEGSASK | |
| Seg | GSPGAGEGTSTEPSEGSAPGTSESTAEAAGCGTAEAATPESGPGTSESATPESGP | 383 |
| 222 | GSEPATSGSETPGSEPATSGTAEAAGCGTAEAATEEGTSTEPSEGSAPGTSTEPS | |
| | EGSAPGSEPATSGSETPTAEAAGCGTAEAASASG | |
| Seg | GSPGAGSEPATSGSETPGTSESATPESGPGSEPATSGSETPGTSESATPESGPGTS | 384 |
| 223 | TEPSEGSAPGTSESATPESGPGSPAGSPTSTEEGSPAGSPTSTEEGSPAGSPTSTE | |
| | EGTSESATPESGPGTSTEPSEGSAPGTSESATPESGPGSEPATSGSETPGTSESAT | |
| | PESGPGSEPATSGSETPGTSESATPESGPGTSTEPSEGSAPTAEAAGCGTAEAAG | |
| | SPAGSPTSTEEGTSESATPESGPGSEPATSGSETPGTSESATPESGPGSPAGSPTST | |
| | EEGSPAGSPTSTEEGTSTEPSEGSAPGTSESATPESGPGTSESATPESGPGTSESA | |
| | TPESGPGSEPATSGSETPGSEPATSGSETPGSPAGSPTSTEEGTSTEPSEGSAPGT | |
| | STEPSEGSAPGSEPATSGSETPGTSESATPESGPGTSASR | |
| Seg | GSPGAGSEPATSGSETPGTSESATPESGPGSEPATSGSETPGTSESATPESGPGTS | 385 |
| 224 | TEPSEGSAPGTSESATPESGPGSPAGSPTSTEEGSPAGSPTSTEEGSPAGSPTSTE | |
| | EGTSESATPESGPGTSTEPSEGSAPGTSESATPESGPGSEPATSGSETPGTSESAT | |
| | PESGPGSEPATSGSETPGTSESATPESGPGTSTEPSEGSAPTAEAAGCGTAEAAG | |
| | SPAGSPTSTEEGTSESATPESGPGSEPATSGSETPGTSESATPESGPGSPAGSPTST | |
| | EEGSPAGSPTSTEEGTSTEPSEGSAPGTSESATPESGTAEAAGCGTAEAAGTSES | |
| | ATPESGPGSEPATSGSETPGSEPATSGSETPGSPAGSPTSTEEGTSTEPSEGSAPG | |
| | TSTEPSEGSAPGSEPATSGSETPGTSESATPESGPGTSASR | |
| Seg | GSPGAGSEPATSGSETPGTSESATPESGPGSEPATSGSETPGTSESATPESGPGTS | 386 |
| 225 | TEPSEGSAPGTSESATPESGPGSPAGSPTSTEEGSPAGSPTSTEETAEAAGCGTA | |
| | EAATSESATPESGPGTSTEPSEGSAPGTSESATPESGPGSEPATSGSETPGTSESA | |
| | TPESGPGSEPATSGSETPGTSESATPESGPGTSTEPSEGSAPTAEAAGCGTAEAA | |
| | GSPAGSPTSTEEGTSESATPESGPGSEPATSGSETPGTSESATPESGPGSPAGSPT | |
| | STEEGSPAGSPTSTEEGTSTEPSEGSAPGTSESATPESGTAEAAGCGTAEAAGTS | |
| | ESATPESGPGSEPATSGSETPGSEPATSGSETPGSPAGSPTSTEEGTSTEPSEGSA | |
| | PGTSTEPSEGSAPGSEPATSGSETPGTSESATPESGPGTSASR | |
| Seg | GSPGAGSPAGSPTSTEEGTSESATPESGPGTSTEPCSGSAPGSPAGSPTSTEEGTS | 387 |
| 226 | TEPSEGSAPGTSTEPCSGSAPGTSESATPESGPGSEPATSGSETPGSEPATCGSET | |
| | PGSPAGSPTSTEEGTSESATPESGPGTESASR | |
| Seg | GSPGAGSPAGSPTSTEEGTSESATPESGPGTSTEGCGESAPGSPAGSPTSTEEGTS | 388 |
| 227 | TEPSEGSAPGTSTEGCGESAPGTSESATPESGPGSEPATSGSETPGSEPAGCGSET | |
| | PGSPAGSPTSTEEGTSESATPESGPGTESASR | |
| Seg | GSPGAGSPAGSPTSTEEGTSESATPESGPGTSTEPSEGSAPGSPAGSPTSGCGETS | 389 |
| 228 | TEPSEGSAPGTSTEGCGESAPGTSESATPESGPSGCGATSGSETPGSEPATSGSET | |
| | PGSPAGSPTSTEEGTSESATPESGPGTESASR | |
| Seg | GSPGAGSPAGSPTSTEEGTSESATPESGPGTSTEPSEGSAPGSPAGSPTSTEEGTS | 390 |
| 229 | TEPSGCGAPGTSTEGCGESAPGTSGCGTPESGPGSEPATSGSETPGSEPATSGSE | |
| | TPGSPAGSPTSTEEGTSESATPESGPGTESASR | |
| Seg | GSPGAGSPAGSPTSTEEGTSESATPESGPGCGTEPSEGSAPGSPGCGPTSTEEGT | 391 |
| 230 | STEPGCGSAPGTESASR | |
| Seg | GSPGAGSCAGSPTSTEEGTSESACSPEGPGTSTEPSEGSCGGPGSPAGSPTSTEE | 392 |
| 231 | GTSESATPESGPGTSTEPSEGSAPGSPAGSPTSTEEGTSTEPSEGSAPGTSTEPSE | |
| | GSAPGTSESATPESGPGSEPATSGSETPGSEPATSGSETPGSPAGSPTSTEEGTSE | |
| | SATPESGPGTSTEPSEGSAPGTSTEPSEGSAPGSPAGSPTSTEEGTSTEPSEGSAP | |
| | GTSTEPSEGSAPGTSESATPESGPGTSTEPSEGSAPGTSESATPESGPGSEPATSG | |
| | SETPGTSTEPSEGSAPGTSTEPSEGSAPGTSESATPESGPGTSESATPESGPGSPA | |
| | GSPTSTEEGTSESATPESGPGSEPATSGSETPGTSESATPESGPGTSTEPSEGSAP | |
| | GTSTEPSEGSAPGTSTEPSEGSAPGTSTEPSEGSAPGTSTEPSEGSAPGTSTEPSE | |
| | GSAPGSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTSTEPSE | |
| | GSAPGTSESATPESGPGSEPATSGSETPGTSESATPESGPGSEPATSGSETPGTSE | |
| | SATPESGPGTSTEPSEGSAPGSPAGSPTSTEEGTSESATPESGPGSEPATSGSETP | |
| | GTSESATPESGPGSPAGSPTSTEEGSPASASR | |
| Seg | GSPGAGSCAGSPTSTEEGTSESACSPEGPGTSTEPSEGSCGGLSGRSDNHSPLGL | 393 |
| 232 | AGSPGSPAGSPTSTEEGTSESATPESGPGTSTEPSEGSAPGSPAGSPTSTEEGTST | |
| | EPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPT | |
| | STEEGTSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPESGPG | |
| | TSESATPESGPGSPAGSPTSTEEGTSESATPESGPGSEPATSGSETPGTSESATPES | |
| | GPGTSTEPSEGSAPGTSTEPSEGSAPGTSTEPSEGSAPGTSTEPSEGSAPGTSTEP | |
| | SEGSAPGTSTEPSEGSAPGSPAGSPTSTEEGTSTEPSEGSAPGTSESATPESGPGS | |
| | EPATSGSETPGTSESATPESGPGSEPATSGSETPGTSESATPESGPGTSTEPSEGS | |
| | APGTSESATPESGPGSPAGSPTSTEEGSPAGSPTSTEEGSPAGSPTSTEEGTSESA | |
| | TPESGPGTSTEPSEGSAPGTSESATPESGPGSEPATSGSETPGTSESATPESGPGS | |
| | EPATSGSETPGTSESATPESGPGTSTEPSEGSAPGSPAGSPTSTEEGTSESATPES | |
| | GPGSEPATSGSETPGTSESATPESGPGSPAGSPTSTEEGSPA | |
| Seg | GSPGAGSCAGSPTSTEEGTSESACSPEGPGTSTEPSEGSCGGLAGRSDNHSPLGL | 394 |
| 233 | AGSPGSPAGSPTSTEEGTSESATPESGPGTSTEPSEGSAPGSPAGSPTSTEEGTST | |
| | EPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPT | |
| | STEEGTSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPESGPG | |
| | TSESATPESGPGSPAGSPTSTEEGTSESATPESGPGSEPATSGSETPGTSESATPES | |
| | GPGTSTEPSEGSAPGTSTEPSEGSAPGTSTEPSEGSAPGTSTEPSEGSAPGTSTEP | |
| | SEGSAPGTSTEPSEGSAPGSPAGSPTSTEEGTSTEPSEGSAPGTSESATPESGPGS | |
| | EPATSGSETPGTSESATPESGPGSEPATSGSETPGTSESATPESGPGTSTEPSEGS | |
| | APGTSESATPESGPGSPAGSPTSTEEGSPAGSPTSTEEGSPAGSPTSTEEGTSESA | |
| | TPESGPGTSTEPSEGSAPGTSESATPESGPGSEPATSGSETPGTSESATPESGPGS | |
| | EPATSGSETPGTSESATPESGPGTSTEPSEGSAPGSPAGSPTSTEEGTSESATPES | |
| | GPGSEPATSGSETPGTSESATPESGPGSPAGSPTSTEEGSPA | |
| Seg | GSPGAGSCAGSPTSTEEGTSESACSPEGPGTSTEPSEGSCGGSPLGLAGSLSGRS | 395 |
| 234 | DNHPGSPAGSPTSTEEGTSESATPESGPGTSTEPSEGSAPGSPAGSPTSTEEGTST | |
| | EPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPT | |
| | STEEGTSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPESGPG | |
| | TSESATPESGPGSPAGSPTSTEEGTSESATPESGPGSEPATSGSETPGTSESATPES | |
| | GPGTSTEPSEGSAPGTSTEPSEGSAPGTSTEPSEGSAPGTSTEPSEGSAPGTSTEP | |
| | SEGSAPGTSTEPSEGSAPGSPAGSPTSTEEGTSTEPSEGSAPGTSESATPESGPGS | |
| | EPATSGSETPGTSESATPESGPGSEPATSGSETPGTSESATPESGPGTSTEPSEGS | |
| | APGTSESATPESGPGSPAGSPTSTEEGSPAGSPTSTEEGSPAGSPTSTEEGTSESA | |
| | TPESGPGTSTEPSEGSAPGTSESATPESGPGSEPATSGSETPGTSESATPESGPGS | |
| | EPATSGSETPGTSESATPESGPGTSTEPSEGSAPGSPAGSPTSTEEGTSESATPES | |
| | GPGSEPATSGSETPGTSESATPESGPGSPAGSPTSTEEGSPA | |
| Seg | GAPCGAGCAGPCGPLAGRSDNHSPLGLAGSPGSPAGSPTSTEEGTSESATPESG | 396 |
| 235 | PGTSTEPSEGSAPGSPAGSPTSTEEGTSTEPSEGSAPGTSTEPSEGSAPGTSESAT | |
| | PESGPGSEPATSGSETPGSEPATSGSETPGSPAGSPTSTEEGTSESATPESGPGTS | |
| | TEPSEGSAPGTSTEPSEGSAPGSPAGSPTSTEEGTSTEPSEGSAPGTSTEPSEGSA | |
| | PGTSESATPESGPGTSTEPSEGSAPGTSESATPESGPGSEPATSGSETPGTSTEPSE | |
| | GSAPGTSTEPSEGSAPGTSESATPESGPGTSESATPESGPGSPAGSPTSTEEGTSE | |
| | SATPESGPGSEPATSGSETPGTSESATPESGPGTSTEPSEGSAPGTSTEPSEGSAP | |
| | GTSTEPSEGSAPGTSTEPSEGSAPGTSTEPSEGSAPGTSTEPSEGSAPGSPAGSPT | |
| | STEEGTSTEPSEGSAPGTSESATPESGPGSEPATSGSETPGTSESATPESGPGSEP | |
| | ATSGSETPGTSESATPESGPGTSTEPSEGSAPGTSESATPESGPGSPAGSPTSTEE | |
| | GSPAGSPTSTEEGSPAGSPTSTEEGTSESATPESGPGTSTEPSEGSAPGTSESATP | |
| | ESGPGSEPATSGSETPGTSESATPESGPGSEPATSGSETPGTSESATPESGPGTST | |
| | EPSEGSAPGSPAGSPTSTEEGTSESATPESGPGSEPATSGSETPGTSESATPESGP | |
| | GSPAGSPTSTEEGSPA | |
| Seg | GAPCGAGCAGPCGPLAGRSDNHSPLGLAGSPGSPAGSPTSTEEGTSESATPESG | 397 |
| 236 | PGTSTEPSEGSAPGSPAGSPTSTEEGTSTEPSEGSAPGTSTEPSEGSAPGTSESAT | |
| | PESGPGSEPATSGSETPGSEPATSGSETPGSPAGSPTSTEEGTSESATPESGPGTS | |
| | TEPSEGSAPG | |
| Seg | GSPGAGSCAGSPTSTEEGTSESACSPEGPGTSTEPSEGSCGGPGSPAGSPTSTEE | 398 |
| 237 | GTSESATPESGPGTSTEPSEGSAPGSPAGSPTSTEEGTSTEPSEGSAPGTSTEPSE | |
| | GSAPGTSESATPESGPGSEPATSGSETPGSEPATSGSETPGSPAGSPTSTEEGTSE | |
| | SATPESGPGTSTEPSEGSAPGTSTEPSEGSAPGSPAGSPTSTEEGTSTEPSEGSAP | |
| | GTSTEPSEGSAPGTSESATPESGPGTSTEPSEGSAPGTSESATPESGPGSEPATSG | |
| | SETPGTSTEPSEGSAPGTSTEPSEGSAPGTSESATPESGPGTSESATPESGPGSPA | |
| | GSPTSTEEGTSESATPESGPGSEPATSGSETPGTSESATPESGPGTSTEPSEGSAP | |
| | GTSTEPSEGSAPGTSTEPSEGSAPGTSTEPSEGSAPGTSTEPSEGSAPGTSTEPSE | |
| | GSAPGSPAGSPTSTEEGTSTEPSEGSAPGTSESATPESGPGSEPATSGSETPGTSE | |
| | SATPESGPGSEPATSGSETPGTSESATPESGPGTSTEPSEGSAPGTSESATPESGP | |
| | GSPAGSPTSTEEGSPAGSPTSTEEGSPAGSPTSTEEGTSESATPESGPGTSTEPSE | |
| | GSAPGTSESATPESGPGSEPATSGSETPGTSESATPESGPGSEPATSGSETPGTSE | |
| | SATPESGPGTSTEPSEGSAPGSPAGSPTSTEEGTSESATPESGPGSEPATSGSETP | |
| | GTSESATPESGPGSPAGSPTSTEEGSPAGSPTSTEEGTSTEPSEGSAPGTSESATP | |
| | ESGPGTSESATPESGPGTSESATPESGPGSEPATSGSETPGSEPATSGSETPGSPA | |
| | GSPTSTEEGTSTEPSEGSAPGSASR | |
| Seg | GSPGATGCGSEPATSGSETPGTSESATPESGPGSEPATSGSETPGTSESATPESGP | 399 |
| 238 | GTSTEPSEGSAPGSPAGSPTSTEEGTSESATPESGPGSEPATSGSETPGTSESATP | |
| | ESGPGSPAGSPTSTEEGSPAGSPTSTEEGTSTEPSEGSAPGTSESATPESGPGTSE | |
| | SATPESGPGTSESATPESGPGSEPATSGSETPGSEPATSGSETPGSPAGSPTSTEE | |
| | GTSTEPSEGSAPGTSTEPSEGSAPGSEPATSGSETPGTSESATPESGPGTSTEPSE | |
| | GSAPSASR | |
| Seg | GSPGAGSCAGSPTSTEEGTSESACSPEGPGTSTEPSEGSCGGLAGRSDNHVPLSL | 400 |
| 239 | SMGPGSPAGSPTSTEEGTSESATPESGPGTSTEPSEGSAPGSPAGSPTSTEEGTST | |
| | EPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPT | |
| | STEEGTSTEPSEGSAPGTSTEPSEGSAPGTSESATPESGPGTSTEPSEGSAPGTSES | |
| | ATPESGPGSEPATSGSETPGTSTEPSEGSAPGTSTEPSEGSAPGTSESATPESGPG | |
| | TSESATPESGPGSPAGSPTSTEEGTSESATPESGPGSEPATSGSETPGTSESATPES | |
| | GPGTSTEPSEGSAPGTSTEPSEGSAPGTSTEPSEGSAPGTSTEPSEGSAPGTSTEP | |
| | SEGSAPGTSTEPSEGSAPGSPAGSPTSTEEGTSTEPSEGSAPGTSESATPESGPGS | |
| | EPATSGSETPGTSESATPESGPGSEPATSGSETPGTSESATPESGPGTSTEPSEGS | |
| | APGTSESATPESGPGSPAGSPTSTEEGSPAGSPTSTEEGSPAGSPTSTEEGTSESA | |
| | TPESGPGTSTEPSEGSAPGTSESATPESGPGSEPATSGSETPGTSESATPESGPGS | |
| | EPATSGSETPGTSESATPESGPGTSTEPSEGSAPGSPAGSPTSTEEGTSESATPES | |
| | GPGSEPATSGSETPGTSESATPESGPGSPAGSPTSTEEGSPA | |

In another aspect, the disclosure provides several XTEN linkers of defined lengths containing a single cysteine residue designed to be incorporated into a fusion protein at the C-terminus of a targeting moiety to permit conjuation of a cross-linker and the resulting TM-linker to the N-terminus of a CCD, the N-terminus of an XTEN, or to a cysteine residue of a cysteine-engineered XTEN of Table 11. The introduction of a reactive thiol that is utilized for conjugation of the targeting moiety to the CCD or to other XTEN (hence, their role as linkers), permits an alternative to creating a single fusion protein comprising the targeting moiety fused to the polypeptide components of the subject targeted conjugate compositions; i.e., the CCD, the PCM and the XTEN. In some cases, the XTEN linkers are designed with H8 tags (SEQ ID NO: 721) to permit recovery of the targeting moiety-linker fusion protein during the processing of the compositions. Non-limiting examples of the XTEN linkers are provided in Table 12, and exemplarly targeted conjugate constructs comprising such targeting moiety-linkers are presented in the Examples, below.

TABLE 12
Cysteine-containing linkers

| Name | | SEQ |
|---|---|---|
| of Cys | | ID |
| Linker | Amino Acid Sequence | NO. |
| X304H8 | GSPGTSESATPESGPGSEPATSGSETPGTSESATPESGPGSEPATSGSETPGTSE | 401 |
| | SATPESGPGTSTEPSEGSAPGSPAGSPTSTEEGTSESATPESGPGSEPATSGSET | |
| | PGTSESATPESGPGSPAGSPTSTEEGSPAGSPTSTEEGTSTEPSEGSAPGTSESA | |
| | TPESGPGTSESATPESGPGTSESATPESGPGSEPATSGSETPGSEPATSGSETPG | |
| | SPAGSPTSTEEGTSTEPSEGSAPGTSTEPSEGSAPGSEPATSGSETPGTSESATP | |
| | ESGPGTSTEPSEGSAPGGAPCGPAGGSSSHHHHHHHH | |
| X304 | GSPGTSESATPESGPGSEPATSGSETPGTSESATPESGPGSEPATSGSETPGTSE | 402 |
| | SATPESGPGTSTEPSEGSAPGSPAGSPTSTEEGTSESATPESGPGSEPATSGSET | |
| | PGTSESATPESGPGSPAGSPTSTEEGSPAGSPTSTEEGTSTEPSEGSAPGTSESA | |
| | TPESGPGTSESATPESGPGTSESATPESGPGSEPATSGSETPGSEPATSGSETPG | |
| | SPAGSPTSTEEGTSTEPSEGSAPGTSTEPSEGSAPGSEPATSGSETPGTSESATP | |
| | ESGPGTSTEPSEGSAPGGAPCGPAGGSSS | |
| X44H8 | GSPGSAGSGSETPGTSESATPESGPGTSTEPSGAPCGPAGGSSSHHHHHHHH | 403 |
| X44 | GSPGSAGSGSETPGTSESATPESGPGTSTEPSGAPCGPAGGSSS | 404 |
| X36H8 | PSEGSAPGSEPATSGSETPGSTAEAAGCGTAEAASAHHHHHHHH | 405 |
| X36 | PSEGSAPGSEPATSGSETPGSTAEAAGCGTAEAASA | 406 |
| X24H8 | TSGSETPGSTAEAAGCGTAEAASAHHHHHHHH | 407 |
| X24 | TSGSETPGSTAEAAGCGTAEAASA | 408 |
| X12H8 | GSTAEAAGCGTAHHHHHHHH | 409 |
| X12 | GSTAEAAGCGTA | 410 |
| CiH8 | GCGHHHHHHHH | 411 |
| Ci | GCG | |

The design, selection, and preparative methods of the invention enable the creation of engineered XTEN that are reactive with electrophilic functionality. The methods to make the subject conjugates provided herein enable the creation of targeted conjugate compositions wherein the payload or targeting moiety molecules are added in a quantified fashion at designated sites. Payloads, targeting moieties and other XTEN may be site-specifically and efficiently linked to the N- or C-terminus of CCD, XTEN, to cysteine-engineered XTEN with a thiol-reactive reagent, or to lysine-engineered XTEN of the disclosure with an amine-reactive reagent, and to an alpha amino group at the N-terminus of a CCD or XTEN, as described more fully, below, and then are purified and characterized using, for example, the non-limiting methods described more specifically in the Examples.

### 4. Length of Sequence

In another aspect, the invention provides XTEN of varying lengths for incorporation into the compositions wherein the length of the XTEN sequence(s) are chosen based on the property or function to be achieved in the composition. For example, XTEN are used as a carrier in the compositions, the invention taking advantage of the discovery that increasing the length of the non-repetitive, unstructured polypeptides enhances the unstructured nature of the XTENs and correspondingly enhances the physicochemical and pharmacokinetic properties of constructs comprising the XTEN carrier. In general, XTEN as monomers or as multimers with cumulative lengths longer that about 400 residues incorporated into the compositions result in longer half-life compared to shorter cumulative lengths, e.g., shorter than about 280 residues. As described more fully in the Examples, proportional increases in the length of the XTEN, even if created by a repeated order of single family sequence motifs (e.g., the four AE motifs of Table 9), result in a sequence with a higher percentage of random coil formation, as determined by GOR algorithm, or reduced content of alpha-helices or beta-sheets, as determined by Chou-Fasman algorithm, compared to shorter XTEN lengths. In addition, increasing the length of the unstructured polypeptide fusion partner, as described in the Examples, results in a construct with a disproportionate increase in terminal half-life compared to polypeptides with unstructured polypeptide partners with shorter sequence lengths. In some embodiments, where the XTEN serve primarily as a carrier, the invention encompasses targeted conjugate compositionscomprising two, three, four or more XTEN wherein the cumulative XTEN sequence length of the XTEN proteins is greater than about 100, 200, 400, 500, 600, 800, 900, or 1000 to about 3000 amino acid residues, wherein the construct exhibits enhanced pharmacokinetic properties when administered to a subject compared to a payload not linked to the XTEN and administered at a comparable dose. In one embodiment of the foregoing, the two or more XTEN sequences each exhibit at least about 80%, 90%, 91%, 92%, 93%, 94%, 95%, 96%, 97%, or 98% or more identity to a sequence selected from any one of Table 10, Table 11, and the remainder, if any, of the carrier sequence(s) contains at least 90% hydrophilic amino acids and less than about 2% of the overall sequence consists of hydrophobic or aromatic amino acids or cysteine. The enhanced pharmacokinetic properties of the targeted conjugate composition, in comparison to payload not linked to the composition, are described more fully, below.

### 5. Net Charge

In other embodiments, the XTEN polypeptides have an unstructured characteristic imparted by incorporation of amino acid residues with a net charge and containing a low percentage or no hydrophobic amino acids in the XTEN sequence. The overall net charge and net charge density is controlled by modifying the content of charged amino acids in the XTEN sequences, either positive or negative, with the net charge typically represented as the percentage of amino acids in the polypeptide contributing to a charged state beyond those residues that are cancelled by a residue with an opposing charge. In some embodiments, the net charge density of the XTEN of the conjugates may be above +0.1 or below −0.1 charges/residue. By “net charge density” of a protein or peptide herein is meant the net charge divided by the total number of amino acids in the protein. In other embodiments, the net charge of an XTEN can be about 0%, about 1%, about 2%, about 3%, about 4%, about 5%, about 6%, about 7%, about 8%, about 9%, about 10% about 11%, about 12%, about 13%, about 14%, about 15%, about 16%, about 17%, about 18%, about 19%, or about 20% or more. Based on the net charge, some XTENs have an isoelectric point (pI) of 1.0, 1.5, 2.0, 2.5, 3.0, 3.5, 4.0, 4.5, 5.0, 5.5, 6.0, or even 6.5. In one embodiment, the XTEN will have an isoelectric point between 1.5 and 4.5 and carry a net negative charge under physiologic conditions.

Since most tissues and surfaces in a human or animal have a net negative charge, in some embodiments the XTEN sequences are designed to have a net negative charge to minimize non-specific interactions between the XTEN containing compositions and various surfaces such as blood vessels, healthy tissues, or various receptors. Not to be bound by a particular theory, an XTEN can adopt open conformations due to electrostatic repulsion between individual amino acids of the XTEN polypeptide that individually carry a net negative charge and that are distributed across the sequence of the XTEN polypeptide. In some embodiments, the XTEN sequence is designed with at least 90% to 95% of the charged residues separated by other non-charged residues such as serine, alanine, threonine, proline or glycine, which leads to a more uniform distribution of charge, better expression or purification behavior. Such a uniform distribution of net negative charge in the extended sequence lengths of XTEN also contributes to the unstructured conformation of the polymer that, in turn, can result in an effective increase in hydrodynamic radius. In preferred embodiments, the negative charge of the subject XTEN is conferred by incorporation of glutamic acid residues. Generally, the glutamic residues are spaced uniformly across the XTEN sequence. In some cases, the XTEN can contain about 10-80, or about 15-60, or about 20-50 glutamic residues per 20 kDa of XTEN that can result in an XTEN with charged residues that would have very similar pKa, which can increase the charge homogeneity of the product and sharpen its isoelectric point, enhance the physicochemical properties of the resulting targeted conjugate composition for, and hence, simplifying purification procedures. For example, where an XTEN with a negative charge is desired, the XTEN can be selected solely from an AE family sequence, which has approximately a 17% net charge due to incorporated glutamic acid, or can include varying proportions of glutamic acid-containing motifs of Table 9 to provide the desired degree of net charge. In one embodiment, an XTEN sequence of Table 10 can be modified to include additional glutamic acid residues to achieve the desired net negative charge. Accordingly, in one embodiment the invention provides XTEN in which the XTEN sequences contain about 1%, 2%, 4%, 8%, 10%, 15%, 17%, 20%, 25%, or even about 30% glutamic acid. In some cases, the XTEN can contain about 10-80, or about 15-60, or about 20-50 glutamic residues per 20kDa of XTEN that can result in an XTEN with charged residues that would have very similar pKa, which can increase the charge homogeneity of the product and sharpen its isoelectric point, enhance the physicochemical properties of the resulting XTEN conjugate composition, and hence, simplifying purification procedures. In one embodiment, the invention contemplates incorporation of up to 5% aspartic acid residues into XTEN in addition to glutamic acid in order to achieve a net negative charge.

Not to be bound by a particular theory, the XTEN of the targeted conjugate compositions with the higher net negative charge are expected to have less non-specific interactions with various negatively-charged surfaces such as blood vessels, tissues, or various receptors, which would further contribute to reduced active clearance. Conversely, it is believed that the XTEN of the targeted conjugate compositions with a low (or no) net charge would have a higher degree of interaction with surfaces that can potentiate the activity of the associated conjugate in the vasculature or tissues.

In other embodiments, where no net charge is desired, the XTEN can be selected from, for example, AG XTEN components, such as the AG motifs of Table 9 that have no net charge. In another embodiment, the XTEN can comprise varying proportions of AE and AG motifs in order to have a net charge that is deemed optimal for a given use or to maintain a given physicochemical property.

The XTEN of the compositions of the present invention generally have no or a low content of positively charged amino acids. In some embodiments, the XTEN may have less than about about 5%, or less than about 2%, or less than about 1% amino acid residues with a positive charge. However, the invention contemplates constructs where a defined number of amino acids with a positive charge, such as lysine, are incorporated into XTEN to permit conjugation between the epsilon amine of the lysine and a reactive group on a payload or a cross-linker to be conjugated to the XTEN backbone. In one embodiment of the foregoing, the XTEN of the subject conjugates has between about 1 to about 10 lysine residues, or about 1 to about 5 lysine residues, or about 1 to about 3 lysine residues, or alternatively only a single lysine residue. Using the foregoing lysine-containing XTEN, conjugates can be constructed that comprise a targeting moiety, or a payload useful in the treatment of a condition in a subject wherein the maximum number of molecules of the payload agent linked to the XTEN component is determined by the numbers of lysines with a reactive side group (e.g., a terminal amine) incorporated into the XTEN.

### 6. Low Immunogenicity

In another aspect, the invention provides XTEN compositions having a low degree of immunogenicity or are substantially non-immunogenic. Several factors can contribute to the low immunogenicity of XTEN, e.g., the non-repetitive sequence, the unstructured conformation, the high degree of solubility, the low degree or lack of self-aggregation, the low degree or lack of proteolytic sites within the sequence, and the low degree or lack of epitopes in the XTEN sequence.

Conformational epitopes are formed by regions of the protein surface that are composed of multiple discontinuous amino acid sequences of the protein antigen. The precise folding of the protein brings these sequences into a well-defined, stable spatial configurations, or epitopes, that can be recognized as “foreign” by the host humoral immune system, resulting in the production of antibodies to the protein or the activation of a cell-mediated immune response. In the latter case, the immune response to a protein in an individual is heavily influenced by T-cell epitope recognition that is a function of the peptide binding specificity of that individual's HLA-DR allotype. Engagement of a MHC Class II peptide complex by a cognate T-cell receptor on the surface of the T-cell, together with the cross-binding of certain other co-receptors such as the CD4 molecule, can induce an activated state within the T-cell. Activation leads to the release of cytokines further activating other lymphocytes such as B cells to produce antibodies or activating T killer cells as a full cellular immune response.

The ability of a peptide to bind a given MHC Class II molecule for presentation on the surface of an APC (antigen presenting cell) is dependent on a number of factors; most notably its primary sequence. In one embodiment, a lower degree of immunogenicity is achieved by designing XTEN sequences that resist antigen processing in antigen presenting cells, and/or choosing sequences that do not bind MHC receptors well. The invention provides substantially non-repetitive XTEN polypeptides designed to reduce binding with MHC II receptors, as well as avoiding formation of epitopes for T-cell receptor or antibody binding, resulting in a low degree of immunogenicity. Avoidance of immunogenicity can attribute to, at least in part, a result of the conformational flexibility of XTEN sequences; i.e., the lack of secondary structure due to the selection and order of amino acid residues. For example, of particular interest are sequences having a low tendency to adapt compactly folded conformations in aqueous solution or under physiologic conditions that could result in conformational epitopes. The administration of polypeptides comprising XTEN, using conventional therapeutic practices and dosing, would generally not result in the formation of neutralizing antibodies to the XTEN sequence, and also reduce the immunogenicity of the payload in the conjugates.

In one embodiment, the XTEN sequences utilized in the subject polypeptides can be substantially free of epitopes recognized by human T cells. The elimination of such epitopes for the purpose of generating less immunogenic proteins has been disclosed previously; see for example WO 98/52976, WO 02/079232, and WO 00/3317 which are incorporated by reference herein. Assays for human T cell epitopes have been described (Stickler, M., et al. (2003) J Immunol Methods, 281: 95-108). Of particular interest are peptide sequences that can be oligomerized without generating T cell epitopes or non-human sequences. This is achieved by testing direct repeats of these sequences for the presence of T-cell epitopes and for the occurrence of 6 to 15-mer and, in particular, 9-mer sequences that are not human, and then altering the design of the XTEN sequence to eliminate or disrupt the epitope sequence. In some embodiments, the XTEN sequences are substantially non-immunogenic by the restriction of the numbers of epitopes of the XTEN predicted to bind MHC receptors. With a reduction in the numbers of epitopes capable of binding to MHC receptors, there is a concomitant reduction in the potential for T cell activation as well as T cell helper function, reduced B cell activation or upregulation and reduced antibody production. The low degree of predicted T-cell epitopes can be determined by epitope prediction algorithms such as, e.g., TEPITOPE (Sturniolo, T., et al. (1999) Nat Biotechnol, 17: 555-61). The TEPITOPE score of a given peptide frame within a protein is the log of the K_{d} (dissociation constant, affinity, off-rate) of the binding of that peptide frame to multiple of the most common human MHC alleles, as disclosed in Sturniolo, T. et al. (1999) Nature Biotechnology 17:555). The score ranges over at least 20 logs, from about 10 to about −10 (corresponding to binding constraints of 10e¹⁰ K_{d} to 10e⁻¹⁰ K_{d}), and can be reduced by avoiding hydrophobic amino acids that serve as anchor residues during peptide display on MHC, such as M, I, L, V, F. In some embodiments, an XTEN component incorporated into a targeted conjugate composition does not have a predicted T-cell epitope at a TEPITOPE threshold score of about −5, or −6, or −7, or −8, or −9, or at a TEPITOPE score of −10. As used herein, a score of “−9” is a more stringent TEPITOPE threshold than a score of −5.

### 7. Increased Hydrodynamic Radius

In another aspect, a subject XTEN useful as a fusion partner has a high hydrodynamic radius; a property that confers a corresponding increased apparent molecular weight to the targeted conjugate composition compared to the payload without the XTEN. As detailed in Example 44, the linking of XTEN to therapeutic protein sequences results in compositions that can have increased hydrodynamic radii, increased apparent molecular weight, and increased apparent molecular weight factor compared to a therapeutic protein not linked to an XTEN. For example, in therapeutic applications in which prolonged half-life is desired, compositions in which one or more XTEN with a high hydrodynamic radius are fused or linked to a targeted conjugate composition can effectively enlarge the hydrodynamic radius of the composition beyond the glomerular pore size of approximately 3-5 nm (corresponding to an apparent molecular weight of about 70 kDa) (Caliceti. 2003. Pharmacokinetic and biodistribution properties of poly(ethylene glycol)-protein conjugates. Adv Drug Deliv Rev 55:1261-1277), resulting in reduced renal clearance of circulating proteins with a corresponding increase in terminal half-life and other enhanced pharmacokinetic properties. The hydrodynamic radius of a protein is conferred by its molecular weight as well as by its structure, including shape or compactness. Not to be bound by a particular theory, the XTEN can adopt open conformations due to the electrostatic repulsion between individual charges of incorporated charged residues in the XTEN as well as because of the inherent flexibility imparted by the particular amino acids in the sequence that lack potential to confer secondary structure. The open, extended and unstructured conformation of the XTEN polypeptide has a greater proportional hydrodynamic radius compared to polypeptides of a comparable sequence length and/or molecular weight that have secondary or tertiary structure, such as typical globular proteins. Methods for determining the hydrodynamic radius are well known in the art, such as by the use of size exclusion chromatography (SEC), as described in U.S. Pat. Nos. 6,406,632 and 7,294,513. Example 51 demonstrates that increases in XTEN length result in proportional increase in the hydrodynamic radius, apparent molecular weight, and/or apparent molecular weight factor to proteins to which they are attached, including scFv, and thus permit the tailoring of a targeted conjugate composition to desired cut-off values of apparent molecular weights or hydrodynamic radii. Accordingly, in certain embodiments, the targeted conjugate composition can be configured with an XTEN such that the resulting composition can have a hydrodynamic radius of at least about 5 nm, or at least about 8 nm, or at least about 10 nm, or about 12 nm, or about 15 nm, or about 20 nm, or about 30 nm or more. As detailed in Example 44, for instance, a scFv of anti-Her2 linked directly to XTEN (without the other components of the CCD and PCM) having 288, 576, or 864 amino acid residues resulted in a determined hydrodynamic radius of 6.7, 8.6, and 9.9; all of which are larger than the known pore size of a renal tubule. In the foregoing embodiments, the large hydrodynamic radius conferred by the XTEN in a targeted conjugate composition can lead to reduced clearance of the resulting conjugate, an increase in terminal half-life, and an increase in mean residence time. As described in the Examples, when the molecular weights of the XTEN-containing compositions are derived from size exclusion chromatography analyses, the open conformation of the XTEN due to the low degree of secondary structure results in an increase in the apparent molecular weight of the conjugates into which they are incorporated. In one embodiment, the present invention makes use of the discovery that the increase in apparent molecular weight can be accomplished by the linking not only of a single XTEN of a given length, but also by the linking of 2, 3, 4 or more XTEN of proportionally shorter lengths, either in linear fashion or as a trimeric or tetrameric, branched configuration, as described more fully, below, and as illustrated in the drawings. In some embodiments, the XTEN comprising a payload and one or more XTEN exhibits an apparent molecular weight of at least about 400 kD, or at least about 500 kD, or at least about 700 kD, or at least about 1000 kD, or at least about 1400 kD, or at least about 1600 kD, or at least about 1800 kD, or at least about 2000 kD. Accordingly, the targeted conjugate composition exhibits an apparent molecular weight that is about 1.3-fold greater, or about 2-fold greater, or about 3-fold greater or about 4-fold greater, or about 8-fold greater, or about 10-fold greater, or about 12-fold greater, or about 15-fold, or about 20-fold greater than the actual molecular weight of the composition. In one embodiment, the isolated targeted conjugate composition of any of the embodiments disclosed herein exhibit an apparent molecular weight factor under physiologic conditions that is greater than about 1.3, or about 2, or about 3, or about 4, or about 5, or about 6, or about 7, or about 8, or about 10, or greater than about 15. In another embodiment, the targeted conjugate composition has, under physiologic conditions, an apparent molecular weight factor that is about 3 to about 20, or is about 5 to about 15, or is about 8 to about 12, or is about 9 to about 10 relative to the actual molecular weight of the composition. Generally, the increased apparent molecular weight of the subject targeted conjugate compositions enhances the pharmacokinetic properties of the composition by a combination of factors, which include reduced active clearance, reduced renal clearance, and reduced loss through capillary and venous junctions.

### 8. Compositions for Increased Expression of XTEN

In another aspect, the invention provides constructs comprising polynucleic acid sequences encoding the fusion proteins of the subject constructs and methods of making the constructs in which additional encoding polynucleotide helper sequences are added to the 5′ end of polynucleotides encoding the fusion proteins or are added to the 5′ end of sequences encoding the fusion protein portion of the subject compositions to enhance and facilitate the expression of the fusion proteins in transformed host cells, such as bacteria. Examples of such encoded helper sequences are given in Table 13 and in the Examples. In one embodiment, the invention provides a polynucleotide sequence construct encoding a polypeptide comprising a helper sequence having at least about 90% sequence identity to a sequence selected from Table 13 linked to the N-terminus of a fusion protein portion of a targeted conjugate composition described herein. The invention provides expression vectors encoding the constructs useful in methods to produce substantially homogeneous preparations of polypeptides and XTEN at high expression levels. In some embodiments, the invention provides methods for producing a substantially homogenous population of polypeptides comprising the fusion protein portion of a targeted conjugate composition, the method comprising culturing in a fermentation reaction a host cell that comprises a vector encoding a polypeptide comprising a helper sequence (wherein the helper sequence has at least 90%sequence identity to a sequence set forth in Table 13) fused to a fusion protein sequence under conditions effective to express the polypeptide such that more than about 2 g/L, or more than about 3 g/L, or more than about 4 g/L, or more than about 5 g/L, or more than about 6 g/L, or more than about 7 grams per liter (7 g/L) of the polypeptide is produced as a component of a crude expression product of the host cell when the fermentation reaction reaches an optical density of at least 130 at a wavelength of 600 nm. In one embodiment, the method further comprises the steps of adsorbing the polypeptide onto a first chromatography substrate under conditions effective to capture an affinity tag of the polypeptide onto the chromatography substrate; eluting and recovering the polypeptide; adsorbing the polypeptide onto a second chromatography substrate under conditions effective to capture the second affinity tag (if present) of the polypeptide onto the chromatography substrate; eluting the polypeptide; and recovering the substantially homogeneous polypeptide preparation. In other embodiments, the invention provides methods for producing a substantially homogenous population of polypeptides comprising a fusion protein of the subject compositions described herein and a first and a second affinity tag and a helper sequence, the method comprising culturing in a fermentation reaction a host cell that comprises a vector encoding a polypeptide comprising an XTEN and the first and second affinity tag under conditions effective to express the polypeptide product at a concentration of more than about 10 milligrams/gram of dry weight host cell (mg/g), or at least about 15 mg/g, or at least about 20 mg/g, or at least about 25 mg/g, or at least about 30 mg/g, or at least about 40 mg/g, or at least about 50 mg/g of said polypeptide when the fermentation reaction reaches an optical density of at least 130 at a wavelength of 600 nm. In one embodiment of the foregoing, the method further comprises the steps of adsorbing the polypeptide onto a first chromatography substrate under conditions effective to capture the first affinity tag of the polypeptide onto the chromatography substrate; eluting and recovering the polypeptide; adsorbing the polypeptide onto a second chromatography substrate under conditions effective to capture the second affinity tag of the polypeptide onto the chromatography substrate; eluting the polypeptide; and recovering the substantially homogeneous polypeptide preparation.

TABLE 13
Examples of helper sequences to facilitate protein
expression, secretion and processing in bacteria

| | SEQ ID |
|---|---|
| Amino Acid Sequence | NO: |
| KNPEQAEEQREET | 412 |
| KNPEQAEEQSEET | 413 |
| KNPEQAEEQAEEQREET | 414 |
| KNPEQAEEQAEEQSEET | 415 |
| KNHEQAEEQAEEQSEET | 416 |
| KKHEQAEEQAEEQSEET | 417 |
| KKPEQAEEQAEEQREET | 418 |
| KNHEQEKEKAEEQSEET | 419 |
| KKQEQEEKKAEEQREET | 420 |
| KNHEKDEKKAEEQSEET | 421 |
| KKQEQEKEQAEEQREET | 422 |
| KNPEQEKEKAEEQREET | 423 |
| KKPEQEEKQAEEQREET | 424 |
| KKQEQEKEQAEEQAESEREET | 425 |
| KKQEQEKEQAEEQSQSQREET | 426 |
| KKQEQEKEQAEEQSESEREET | 427 |
| KKQEQEKEQAEEQAKAESEAEREET | 428 |
| KKQEQEKEQAEEQSKSQAEAEREET | 429 |
| KKQEQEKEQAEEQAQAQAEDEREET | 430 |
| KKQEQEKEQAEEQSKSKAEDEREET | 431 |

### IV). Payloads

The present invention relates in part to targeted conjugate compositions comprising one or more payload molecules. It is contemplated that subject compositions can be linked to a broad diversity of payload molecules, including biologically active peptides, proteins, pharmacologically active small-molecules, and imaging small-molecule payloads, as well as combinations of these types of payloads resulting in compositions with 1, 2, 3, 4 or more types of payloads. More particularly, the active payload may fall into one of a number of structural classes, including but not limited to small molecule drugs, biologically active proteins (peptides, polypeptides, proteins, recombinant proteins, antibodies, and glycoproteins), steroids, and the like. In some embodiments, the invention addresses a long-felt need in both increasing the terminal half-life of exogenously administered therapeutic and diagnostic payloads as well as improving the therapeutic index and reducing side effects and damage caused by such payloads to healthy tissues in a subject in need thereof.

Non-limiting examples of functional classes of pharmacologically active payload agents for use in linking to subject composition of the invention may be any one or more of the following: anti-inflammatories, anti-cancer agents, cytotoxic drugs, neoplastics, antineoplastics, diagnostic agents, contrasting agents, and radioactive imaging agents. In some preferred embodiments, the payloads are cytotoxic or anti-cancer agents, including but not limited one or more drugs and/or biologics selected from the group consisting of the drugs set forth in Tables 14-17. In other preferred embodiments, the payloads are anti-inflammatory agents, including but not limited to one or more drugs selected from the group consisting of the drugs set forth in Table 17.

For the targeted conjugate composition, it is specifically contemplated that a payload can be a pharmacologically active agent that possesses a suitably reactive functional group, including, but not limited to a native amino group, a sulfydryl group, a carboxyl group, an aldehyde group, a ketone group, an alkene group, an alkyne group, an azide group, an alcohol group, a heterocycle, or, alternatively, is modified to contain at least one of the foregoing reactive groups suitable for coupling to either an XTEN, XTEN-cross-linker, or XTEN-click-chemistry reactant of the invention using any of the conjugation methods described herein or are otherwise known to be useful in the art for conjugating such reactive groups. Specific functional moieties and their reactivities are described in Organic Chemistry, 2nd Ed. Thomas Sorrell, University Science Books, Herndon, Va. (2005). Further, it will be understood that any payload containing a reactive group or that is modified to contain a reactive group will also contain a residue after conjugation to which either the XTEN, the XTEN-cross-linker, or the XTEN-click-chemistry reactant is linked.

Exemplary payloads suitable for covalent attachment to either an XTEN polymer, XTEN-cross-linker, or XTEN-click-chemistry reactant include biologically active proteins and pharmacologically active small molecule drugs with activity. Exemplary drugs suitable for the inventive compositions can be found as set forth in the official United States Pharmacopeia, official Homeopathic Pharmacopeia of the United States, or official National Formulary, in the Physician's Desk Reference (PDR) and in the Orange Book maintained by the U.S. Food and Drug Administration (FDA). Preferred drugs are those having the needed reactive functional group or those that can be readily derivatized to provide the reactive functional group for conjugation and will retain at least a portion of the pharmacologic activity of the unconjugated payload when conjugated to XTEN.

1. Drugs as Payloads

In one aspect of the invention, the drug payload for the targeted conjugate compositions for conjugation to the CCD described herein is one or more agents described herein or selected from one or more drugs or biologics selected from the group consisting of the compounds set forth in Tables 14-17, or a pharmaceutically acceptable salt, acid or derivative or agonist thereof. In one embodiment, the payload is one or more cytotoxic agents selected from the group consisting of the drugs set forth in Table 15. In one embodiment, the payload for incorporation into the targeted conjugate composition is one or more anti-inflammatory agents selected from the group consisting of the drugs set forth in Table 17. In another embodiment, the payload is one or more biologic agents selected from the group consisting of the biologics set forth in Table 16. In some embodiments, the drug is derivatized to introduce a reactive group for conjugation to the subject XTEN, the XTEN-cross-linkers, or the XTEN-click-chemistry reactants described herein. In another embodiment, the drug for conjugation is derivatized to introduce a cleavable linker such as, but not limited to, valine-citrulline-PAB, wherein the linker is capable of being cleaved by a circulating or an intracellular protease after administration to a subject, thereby freeing the drug payload from the conjugate.

TABLE 14
Drug Payloads for Conjugation to XTEN
Drugs

Erlotinib; Bortezomib; Alitretinoin, Allopurinol, arsenic trioxide, clofarabine, dexrazoxane,
Fulvestrant; Sutent (SU11248), Letrozole; Imatinib mesylate; PTK787/ZK 222584; Bendamustine;
Romidepsin; Pralatrexate; Cabazitaxel (XRP-6258); Everolimus (RAD-001); Abirateron; Oxaliplatin;
5-FU (5-fluorouracil), leucovorin, rapamycin; lapatinib; lonafarnib; sorafenib; gefitinib;
cyclosphosphamide; busulfan; improsulfan; piposulfan; benzodopa; carboquone; meturedopa;
uredopa; altretamine; triethylenemelamine; triethylenephosphoramide; triethylenethiophosphoramide;
trimethylomelamine; bullatacin; bullatacinone; camptothecin; topotecan; bryostatin; callystatin; CC-
1065; adozelesin; calicheamycin; auristatin; monomethyl auristatin E (MMAE); monomethyl
auristatin F (MMAF); (valine-citrulline-PAB)-monomethyl auristatin E; (valine-citrulline-PAB)-
monomethyl auristatin F; carzelesin; bizelesin; cryptophycins (particularly cryptophycin 1 and
cryptophycin 8); dolastatin; duocarmycin; eleutherobin; pancratistatin; sarcodictyin; spongistatin;
chlorambucil; chlornaphazine; cholophosphamide; estramustine; ifosfamide; mechlorethamine;
mechlorethamine oxide hydrochloride; melphalan; novembichin; phenesterine; prednimustine;
trofosfamide; uracil mustard; carmustine; chlorozotocin; fotemustine; lomustine; nimustine;
ranimnustine; calicheamicin; dynemicin; dynemicin A; clodronate; esperamicin; neocarzinostatin
chromophore; aclacinomysins, actinomycin; anthramycin; azaserine; bleomycin; cactinomycin;
carabicin; carminomycin; carzinophilin; chromomycinis; dactinomycin; daunorubicin; detorubicin; 6-
diazo-5-oxo-L-norleucine; doxorubicin; morpholino-doxorubicin; lenalidomide, cyanomorpholino-
doxorubicin; (valine-citrulline-PAB)-doxorubicin; 2-pyrrolino-doxorubicin and deoxydoxorubicin;
epirubicin; esorubicin; idarubicin; marcellomycin; mitomycin C; mycophenolic acid; nogalamycin;
olivomycin; peplomycin; potfiromycin; puromycin; quelamycin; rodorubicin; streptonigrin;
streptozocin; tubercidin; ubenimex; zinostatin; zorubicin; 5-fluorouracil (5-FU); fdenopterin;
methotrexate; pteropterin; trimetrexate; fludarabine; 6-mercaptopurine; thiamiprine; ancitabine;
azacitidine; 6-azauridine; carmofur; cytarabine; dideoxyuridine; doxifluridine; enocitabine;
meclorethamine, floxuridine; calusterone; dromostanolone propionate; epitiostanol; mepitiostane;
testolactone; aminoglutethimide; trilostane; frolinic acid; aceglatone; aldophosphamide glycoside;
aminolevulinic acid; eniluracil; amsacrine; bestrabucil; bisantrene; edatraxate; defofamine;
demecolcine; diaziquone; elformithine; elliptinium acetate; an epothilone; etoglucid; gallium nitrate;
hydroxyurea; lentinan; lonidainine; maytansine; ansamitocins; mitoguazone; mopidanmol; nitraerine;
pentostatin; phenamet; pirarubicin; losoxantrone; methoxsalen, podophyllinic acid; 2-ethylhydrazide;
procarbazine; razoxane; rhizoxin; ribavirin; zidovudine; acyclovir; gangcyclovir; vidarabine;
idoxuridine; trifluridine; foscarnet; amantadine; rimantadine; saquinavir; indinavir; ritonavir; alpha-
interferons and other interferons; AZT; sizofuran; spirogermanium; tenuazonic acid; triaziquone;
2; 2′,2″-trichlorotriethylamine; T-2 toxin; verracurin A; roridin A; anguidine); urethan; vindesine;
dacarbazine; mannomustine; mitobronitol; mitolactol; pipobroman; gacytosine; arabinoside (“Ara-
C”;); cyclophosphamide; thiotepa; taxoids; epaclitaxel; paclitaxel; docetaxel; doxetaxel; irinotecan;
pemetrexed; chloranbucil; gemcitabine; 6-thioguanine; cisplati; carboplatin; vinblastine; platinum;
etoposide, VP-16; ifosfamide; mitoxantrone; novantrone; teniposide; edatrexate; daunomycin;
aminopterin; xeloda; ibandronate; CPT-11; topoisomerase inhibitor RFS 2000;
difluoromethylornithine (DMFO); retinoic acid; capecitabine; mesna, lidocaine; bupivacaine;
memantine; quinacrine, donepezil; rivastigmine; galantamine; morphine; oxycodone;
hydromorphone; oxymorphone; metopon; apomorphine; normorphine; etorphine; buprenorphine;
meperidine; lopermide; anileridine; ethoheptazine; piminidine; betaprodine; diphenoxylate; fentanil;
sufentanil; alfentanil; remifentanil; levorphanol; dextromethorphan; phenazocine; pentazocine;
cyclazocine; methadone; isomethadone; propoxyphene; naloxone; naltrexone; treprostinil; N-
methylnaloxone; 6-amino-14-hydroxy-17-allylnordesomorphine; naltrendol;, N-methylnaltrexone;
nalbuphine; butorphanol; cyclazocine; pentazocine,; nalmephene; naltrindole; nor-binaltorphimine;
oxilorphan; 6-amino-6-desoxo-naloxone; pentazocine; levallorphanmethylnaltrexone; buprenorphine;
cyclorphan; levalorphan; cyclosporine; cyclosporine A; mycophenylate mofetil; sirolimus; tacrolimus;
prednisone; azathioprine; cyclophosphamide; prednisone; aminocaproic acid; chloroquine;
hydroxychloroquine; dexamethasone; chlorambucil; danazol; bromocriptine; Nilotinib (AMN107);
Nelarabine, amifostine, amiodarone, aminocaproic acid, aminohippurate sodium, aminoglutethimide,
aminolevulinic acid, aminosalicylic acid, amsacrine, anagrelide, anastrozole, asparaginase,
anthracyclines, bexarotene, bicalutamide, bleomycin, buserelin, busulfan, cabergoline, capecitabine,
carboplatin, carmustine, chlorambucin, cilastatin sodium, cisplatin, cladribine, clodronate,
cyclophosphamide, cyproterone, cytarabine, camptothecins, 13-cis retinoic acid, all trans retinoic
acid; dacarbazine, dactinomycin, daunorubicin, deferoxamine, dexamethasone, diclofenac,
diethylstilbestrol, docetaxel, doxorubicin, epirubicin, estramustine, etoposide, exemestane,
fexofenadine, fludarabine, fludrocortisone, fluorouracil, fluoxymesterone, flutamide, gemcitabine,
epinephrine, L-Dopa, hydroxyurea, idarubicin, ifosfamide, imatinib, irinotecan, itraconazole,
goserelin acetate, letrozole, leucovorin, levamisole, lisinopril, lovothyroxine sodium,
mechlorethamine, medroxyprogesterone, megestrol, melphalan, metaraminol bitartrate,
metoclopramide, mexiletine, mitomycin, mitotane, naloxone, nicotine, nilutamide, octreotide,
pamidronate, pilcamycin, porfimer, prednisone, prochlorperazine, ondansetron, raltitrexed, sirolimus,
tacrolimus, tamoxifen, temozolomide, testosterone, tetrahydrocannabinol, thalidomide, thioguanine,
topotecan, tretinoin, valrubicin, vincristine, vindesine, vinorelbine, dolasetron, granisetron;
formoterol, fluticasone, leuprolide, midazolam, alprazolam, amphotericin B, podophylotoxins,
nucleoside antivirals, aroyl hydrazones, sumatriptan; macrolides such as erythromycin, oleandomycin,
troleandomycin, roxithromycin, clarithromycin, davercin, azithromycin, flurithromycin,
dirithromycin, josamycin, spiromycin, midecamycin, leucomycin, miocamycin, rokitamycin,
andazithromycin, and swinolide A; fluoroquinolones such as ciprofloxacin, ofloxacin, levofloxacin,
trovafloxacin, alatrofloxacin, moxifloxicin, norfloxacin, enoxacin, grepafloxacin, sunitinib,
gatifloxacin, lomefloxacin, sparfloxacin, temafloxacin, pefloxacin, amifloxacin, fleroxacin,
tosufloxacin, prulifloxacin, irloxacin, pazufloxacin, clinafloxacin, and sitafloxacin; aminoglycosides
such as gentamicin, netilmicin, paramecin, tobramycin, amikacin, kanamycin, neomycin, and
streptomycin, vancomycin, teicoplanin, rampolanin, mideplanin, colistin, daptomycin, gramicidin,
colistimethate; polymixins such as polymixin B, capreomycin, bacitracin, penems; penicillins
including penicllinase-sensitive agents like penicillin G, penicillin V; penicllinase-resistant agents like
methicillin, oxacillin, cloxacillin, dicloxacillin, floxacillin, nafcillin; gram negative microorganism
active agents like ampicillin, amoxicillin, and hetacillin, cillin, and galampicillin; antipseudomonal
penicillins like carbenicillin, ticarcillin, azlocillin, mezlocillin, and piperacillin; cephalosporins like
cefpodoxime, cefprozil, ceftbuten, ceftizoxime, ceftriaxone, cephalothin, cephapirin, cephalexin,
cephradrine, cefoxitin, cefamandole, cefazolin, cephaloridine, cefaclor, cefadroxil, cephaloglycin,
cefuroxime, ceforanide, cefotaxime, cefatrizine, cephacetrile, cefepime, cefixime, cefonicid,
cefoperazone, cefotetan, cefinetazole, ceftazidime, loracarbef, and moxalactam, monobactams like
aztreonam; and carbapenems such as imipenem, meropenem, pentamidine isethiouate, albuterol
sulfate, lidocaine, metaproterenol sulfate, beclomethasone diprepionate, triamcinolone acetamide,
budesonide acetonide, fluticasone, ipratropium bromide, flunisolide, cromolyn sodium, and
ergotamine tartrate; taxanes such as paclitaxel; SN-38, tyrphostines, 20-epi-1,25 dihydroxy
vitaminD3, 5-ethynyluracil, abiraterone, Acivicin, Aclarubicin, Acodazole Hydrochloride, AcrQnine,
acylfulvene, adecypenol, adramycin, Aldesleukin, ALL-TK antagonists, ambamustine, amidox,
Ambomycin, Ametantrone Acetate, amrubicin, andrographolide, angiogenesis inhibitors, antagonist
D, antagonist G, antarelix, anti-androgen, anti-dorselizing morphogenetic protein-1, anti-estrogen,
antimetabolites, anti-neoplaston, anti-oestrogens, anti-sense oligonucleotides, anti-venom,
aphidicolinglycinate, apoptosis gene modulators, apoptosis regulators, apurinic acid, ara-CDP-DL-
PTBA, arginine deaminase, Asperlin, asulacrine, atamestane, atrimustine, atrsacrine, axinastatin 1,
axinastatin 2, axinastatin 3, azasetron, azatoxin, azatyrosine, Azptepa: Azotomycin, baccatin III
derivatives, balanol, Batimastat, BCR/ABLantagonists, benzochlorins, Benzodepa,
benzoylstaurosporine, staurosporine, beta-alethine, betaclamycin B, betalactamderivatives,
betamethasone, betulinic acid, bFGFinhibitor, Bicalutamide, Bisantrene Hydrochloride,
bisaziridinylspermine, bisnafide, Bisnafide Dimesylate, bistrateneA, Bleomycin Sulfate, breflate,
Brequinar Sodium, bromine epiandrosterone, Bropirimine, budotitane, buthionine sulfoximine,
calcipotriol, calphostin C, camptothecin derivatives, canarypox IL-2, capedtabine, Caracemide,
Carbetimer, carboxamide-amino-triazole: carboxyamidotriazole, CaRestM3, CARN700, cartilage
derived inhibitor, Carubicin Hydrochloride, casein kinase inhibitors(ICOS), castanospermine,
cecropin B, Cedefingol, cetrorelix, chlorins, chloroquinoxaline sulfonamide, , chlorotrianisene,
cicaprost, Cirolemycin, cis-porphyrin, clomifene analogues, clotrimazole, collismycin A, collismycin
B, combretastatin A4, combretastatin analogue, conagenin, crambescidin 816, crisnatol, Crisnatol
Mesylate, cryptophycin 8, cryptophycin A derivatives, curacin A, cyclopentanthraquinones,
cycloplatam, cypemycin, cytarabineocfosfate, cytolyticfactor, cytostatin, cytotoxic agents,
Daunorubicin Hydrochloride, Decitabine, dehydrodidemnin B, deslorelin, dexifosfamide,
Dexormaplatin, dexrmzoxane, dexverapamil, Dezaguanine, Dezaguanine Mesylate, DHEA,
diaziquorie, dicarbazine, didemnin 13, didox, diethylnorspermine, dihydro-5-
azacytidine: dihydrotaxol,9-, dioxamycin, diphenylspiromustine, docosanol, Doxorubicin
Hydrochloride, Droloxifene, Droloxifene Citrate, Dromostanolone Propionate, dronabinol,
Duazomycin, duocannycin SA, ebselen, ecorustine, edelfosine, edrocolomab, Eflomithine
Hydrochloride, eflornithine, elemene, Elsamitrucin, emitefur, Enloplatin, Enpromate, epiandrosterone,
Epipropidine, Epirubicin Hydrochloride, epristeride, Erbulozole, erythrocyte gene therapy, Esorubicin
Hydrochloride, estramustine analogue, estrogen agonists, estrogen antagonists, Etanidazole,
ethinyloestradiol, Ethiodized Oil I131, Etoposide Phosphate, Etoprine, fadrozole, Fadrozole
Hydrochloride, Fazarabine, fazarabine, fenretinide, Fenretinide: Floxuridine, finasteride, flavopiridol,
flezelastine, Fludarabine Phosphate, fluorodaunorunicin hydrochloride, Flurocitabine, forfenimex,
formestane, Fosquidone, fostriecin, Fostriecin Sodium, gadoliniumtexaphyrin, galocitabine, ganirelix,
gelatinase inhibitors: gemcitabine, Gemcitabine Hydrochloride, glutathione inhibitors, Gold Au198,
goserelin, hepsulfam, heregulin, hexamethylenebisacetamide, hexamethylmelamine, human chorionic
gonadotrophin: monophosphoryl lipid A + myobacterium cell walls k, hypericin, ibandronic acid,
Idarubicin Hydrochloride, idoxifene, idramantone, Ilmofosine, ilomastat, imidazoacridones,
imiquimod, immuno stimulant peptides: insulin-like growth factor-1 receptor inhibitor, interferon
agonists, Interferon Alfa-2a, Interferon Alfa-2b, Interferon Alfa-n1, Interferon Alfa-n3, Interferon
Beta-Ia, Interferon Gamma-Ib, iobenguane, iododoxorubicin, ipomeanol, Iproplatin, Irinotecan
Hydrochloride, iroplact, irsogladine, isobengazole, isohomohalicondrin B, itasetron, jasplakinolide,
kahalalide F, lamellarin-Ntriacetate, lanreotide, Lanreotide Acetate, leinamycin, lenograstim, lentinan
sulfate, leptolstatin, leukemia inhibiting factor, leukocyte alpha interferon, Leuprolide Acetate,
leuprolide + estrogen + progesterone, leuprorelin, liarozole, Liarozole Hydrochloride, linear polyamine
analogue, lipophilic disaccharide peptide, lipophilic platinum compounds, lissoclinamide7, lobaplatin,
lombricine, lometrexol, Lometrexol Sodium, lonidamine, Losoxantrone Hydrochloride, lovastatin,
loxoribine, luprolide, lurtotecan, lutetlumtexaphyrin, lysofylline, lyticpeptides, maitansine,
mannostatin A, marimastat, Masoprocol, maspin, matrilysin inhibitors, matrix metallo proteinase
inhibitors, mecaptopurine, Mechlorethamine Hydrochloride, Megestrol Acetate, Melengestrol
Acetate, Menogaril, merbarone, meterelin, methioninase, methlorethamine, Metoprine, Meturedepa,
microalgal, mifepristone, MIFinhibitor, miltefosine, mirimostim, mismatched double stranded RNA,
Mitindomide, Mitocarcin, Mitocromin, Mitogillin, mitoguazone, Mitomalcin, mitomycin analogues,
mitonafide, Mitosper, mitotoxin fibroblast growth factor-saporin, mofarotene, molgramostim,
monoclonal antibody, multiple drug resistance gene inhibitor, multiple tumor suppressor1-based
therapy, mustard anticancer agent, mutamycin, mycaperoxide B, mycobacterial cell wall extract,
Mycobacterium bovis, myriaporone, N-acetyldinaline: N-substitutedbenzamides, nafarelin, nagrestip,
naloxone + pentazocine, napavin, naphterpin, nartograstim, nedaplatin, nemorubicin, neridronic acid,
neutral endo peptidase, nisamycin, nitric oxide modulators, nitrogen mustard derivatives, nitroxide
antioxidant, nitrullyn, Nocodazole: Nogalamycin, O6-benzylguanine, oestradiol, okicenone,
oligonucleotides, onapristone: ondansetron, ondansetron, oracin, oral cytokine inducer, Ormaplatin,
osaterone, oxaunomycin, Oxisuran, palauamine, palmitoylrizoxin, pamidronic acid, panaxytriol,
panomifene, parabactin, pazelliptine: pegaspargase, Pegaspargase,
peldesine: pentosanpolysulfatesodium, Peliomycin, Pentamustine, pentrozole, Peplomycin Sulfate,
perflubron, Perfosfamide, perillyl alcohol, phenazinomycin, phenylacetate, phosphatase inhibitors,
picibanil, pilocarpine hydrochloride, piritrexim, Piroxantrone Hydrochloride, placetin A, placetin B,
plasminogen activator inhibitor, platinum complex, platinum compounds, platinum-triamine complex,
Plicamycin, Plomestane, Porfimer Sodium, Procarbazine Hydrochloride, propylbis-acridone,
prostaglandin J2, prostatic carcinoma, proteasome inhibitors, protein A-based immune modulator,
protein kinase C inhibitor, protein tyrosine phosphatase inhibitors, purine nucleoside phosphorylase
inhibitors, Puromycin Hydrochloride, purpurins, Pyrazofurin, pyrazoloacridine, pyridoxylated
hemoglobin polyoxyethylene conjugate, raf antagonists, ramosetron, rasfarnesyl protein transferase
inhibitors, ras inhibitors: ras-GAP inhibitor, retelliptine demethylated, rhenium Re186 etidronate,
Riboprine, ribozymes, RII retinamide, RM-131 (ghrelin agonist), RM-493 (agonis for melanocortin
type 4 receptor), Rogletimide, rohitumine, romurtide, roquinimex, rubiginone B1, ruboxyl, Safingol
Hydrochloride, Safingol, saintopin: Sar CNU, sarcophytol A, sargramostim, Sdi1 mimetics,
Semustine, senescence derived inhibitor 1, sense oligonucleotides, signal transduction inhibitors,
signal transduction modulators, Simtrazene, single chain antigen binding protein, sobuzoxane, sodium
borocaptate, sodium phenylacetate, solverol, somatomedin binding protein, sonermin, Sparfosate
Sodium, sparfosic acid, Sparsomycin, Spirogermanium Hydrochloride, Spiromustine,
spiromustine: splenopentin, Spiroplatin, splcamycin D, squalamine, stem cell inhibitor, stem-cell
division inhibitors, stipiamide, stromelysin inhibitors, Strontium Chloride Sr89, sulfmosine,
Sulofenur, superactive vasoactive intestinal peptide antagonist, suradista, suramin, swainsonine,
synthetic glycosamino glycans, Talisomycin, tallimustine, tamoxifen methiodide, tauromustine,
tazarotene, Tecogalan Sodium, Tegafur, tellurapyrylium, telnoporfin, telomerase inhibitors,
Teloxantrone Hydrochloride, Temoporfin, ternozolomide, Teroxirone, tetrachlorodecaoxide,
tetrazomine, texotere, thallblastine, thiocoraline, thrombopoietin mimetic, thymalfasin, thymopoietin
receptor agonist, thymotrinan, thyroid stimulating hormone, Tiazofurin, tinethylotiopurpurin,
Tirapazamine, titanocene dichloride, Topotecan Hydrochloride, topsentin, toremifene, Toremifene
Citrate, totipotent stem cell factor, translation inhibitors, Trestolone Acetate, triacetyluridine,
triciribine, Triciribine Phosphate: Trimetrexate, Trimetrexate Glucuronate, Triptorelin,
triptorelin: tropisetron, tubulozole hydrochloride, turosteride, tyrosine kinase inhibitors, UBC
inhibitors, urodepa, urogenital sinus-derived growth inhibitory factor, urokinase receptor antagonists,
vapreotide, variolin B, vector system, velaresol, venom, veramine, verdins, Verleporfin, verteporfin,
Vinblastine Sulfate, vincristine sulfate, vindesine, Vindesine Sulfate, Vinepidine Sulfate, Vinglycinate
Sulfate, Vinleurosine Surfate, vinorelbine tartrate, vinrosidine sulfate, vinxaltine, Vinzolidine Surfate,
vitaxin, Vorozole, zanoterone, Zeniplatin, zilascorb, zinostatin stimalamer, Zorubicin Hydrochloride,
Bovine pancreatic RNase, Human pancreatic RNAse, Mammalian pancreatic RNase, onconase,
ranpirnase, pokeweed antiviral protein, rachelmycin, ricin-A chain, gelonin, everolimus, carfilzomib,
tubulysin, tubulysin B, tubulysin M, maytansinoid DM1, maytansinoid DM4, triptolide, SJG-136,
apaziquone, irofulven, illudin S, tomaymycin, zoledronate

TABLE 15
Cytotoxic Drugs for Conjugation as Payloads to XTEN
Drugs

doxorubicin, nemorubicin, PNU-159682, paclitaxel, docetaxel,
auristatin E, auristatin F, dolastatin 10, dolastatin 15,
monomethyl auristatin E (MMAE), monomethyl auristatin F
(MMAF), monomethyl auristatin D (MMAD), maytansine,
mertansine (DM1), maytansinoid DM4, calicheamicin,
N-acetyl-calicheamicin, vinblastine, vincristine, vindesine,
vinorelbine, camptothecin, topotecan, irinotecan, SN-38,
duocarmycin A, duocarmycin B1, duocarmycin B2, duocarmycin
C1, duocarmycin C2, duocarmycin D, duocarmycin SA, duocarmycin
TM, duocarmycin MB, duocarmycin DM, mitomycin C, rachelmycin,
epothilone A, epothilone B, epothilone C, tubulysin B, tubulysin
M, pyrrolobenzodiazepine (PBD), bortezomib

TABLE 16
Biologically Active Proteins as Payloads for Linking to XTEN
Protein/Peptide Name

hTNF, IL-12, ranpirnase, human ribonuclease (RNAse), Bovine pancreatic
RNase, pokeweed antiviral protein, Pseudomonas exotoxin A, gelonin,
ricin-A, interferon-alpha, interferon-lambda, urease, amatoxin, alpha-
amanitin, beta-amanitin, gamma-amanitin, epsilon-amanitin, bouganin,
staphylococcal enterotoxin

TABLE 17
Anti-inflammatory Drugs as Payloads for Conjugation to XTEN
Drugs

dexamethasone, indomethacin, prednisolone, betamethasone dipropionate,
clobetasol propionate, fluocinonide, flurandrenolide, halobetasol
propionate, diflorasone diacetate, desoximetasone

2. Nucleic Acids as Payloads

The invention also contemplates the use of nucleic acids as payloads in the XTEN conjugates. In one embodiment, the invention provides targeted conjugate compositions wherein the payload is selected from the group consisting of aptamers, antisense oligonucleotides, ribozyme nucleic acids, RNA interference nucleic acids, and antigene nucleic acids. Such nucleic acids used as therapeutics are know in the art (Edwin Jarald, Nucleic acid drugs: a novel approach. African Journal of Biotechnology Vol. 3 (12):662-666, 2004; Joanna B. Opalinska. Nucleic-acid therapeutics: basic principles and recent applications. Nature Reviews Drug Discovery 1:503-514, 2002).

### V). Targeting Moieties and Methods of Making Such Compositions 1. Antibody Fragments as Targeting Moieties

The present invention relates, in part, to targeted conjugate compositions comprising targeting moieties (TM) comprising antibodies or antibody fragments derived from antibodies recombinantly fused or chemically conjugated to one or more extended recombinant polypeptides (“XTEN”). In particular, the invention provides isolated targeted conjugate compositions comprising such TM that are useful in the treatment of diseases, disorders or conditions in which the targeting moiety can be directed to an antigen, ligand, or receptor implicated in, associated with, or that modulates a disease, disorder or condition, while the XTEN carrier portion can be designed to confer a desired half-life or enhanced pharmaceutical property through the payload components on the targeted conjugate compositions, as described more fully above. In one embodiment, the composition can further comprise a second targeting moiety or multiple targeting moieties that can have binding affinity for the same or a different target, resulting in multivalent or multispecific targeting moieties, respectively. The invention provides several different forms and configurations of targeting moieties and XTEN. The targeted conjugate compositions of the embodiments disclosed herein exhibit one or more or any combination of the properties and/or the embodiments as detailed herein.

In general, the targeting moieties of the subject targeted conjugate compositions exhibit a binding specificity to a given target tissue or cell when used in vivo or when utilized in an in vitro assay. The subject targeted conjugate compositions comprising two or more targeting moieties can be designed to bind the same target epitope, different epitopes on the same target, or different targets by the selective incorporation of targeting moieties with binding affinity to the respective binding sites.

The targets to which the targeting moieties of the subject targeted conjugate compositions can be directed include cytokines, cytokine-related proteins, cytokine receptors, chemokines, chemokines receptors, cell surface receptors or antigens, hormones or similar circulating proteins or peptides, oligonucleotides, or enzymatic substrates, or small organic molecules, haptens or drugs. The targets are generally associated with a disease, disorder or condition. As used herein, “a target associated with a disease, disorder or condition” means that the target is either expressed or overexpressed by disease cells or unhealthy tissues, the target causes or is a mediator or is a by-product of the disease, disorder or condition, or the target is generally found in higher concentrations in a subject with the disease, disorder or condition compared to a healthy tissue or subject, or the target is found in higher than baseline concentrations within or proximal to the areas of the disease, disorder or condition in the subject. A target may also be a distinctive epitope, ligand or chemical entity associated with a disease, disorder or condition notwithstanding any overabundance or quantity in diseased versus normal tissue (e.g., EGFR VIII variant). A non-limiting example of the foregoing is the target HER2, which is implicated in approximately 30 percent of breast cancers due to an amplification of the HER2/neu gene or over-expression of its protein product. Over-expression of the HER2 receptor in breast cancer is associated with increased disease recurrence and worse prognosis, and a humanized anti-Her2/neu antibody is used in treatment of breast cancers expressing the HER2 receptor (see for example U.S. Pat. No. 4,753,894).

In one embodiment, the one or more targeting moieties of the targeted conjugate compositions can have binding affinity to one or more tumor-associated antigens (TAA) or ligands known to be expressed on tumor or cancer cells or are otherwise associated with tumors or cancers. Tumor-associated antigens are known in the art, and are generally regarded as effective cellular targets for cancer diagnosis and therapy. In particular, researchers have sought to identify TAA that are specifically expressed on the surface of one or more particular types of cancer cell as compared to on one or more normal non-cancerous cells, and has given rise to the ability to specifically target cancer cells for destruction via antibody-based therapies. In one embodiment, the one or more targeting moieties of the targeted conjugate compositions have binding affinity to targets and ligands selected from, but not limited to the targets of Table 2, Table 3, Table 4, or Table 18.

As described more fully below, the targeting moieties can be derived from or based on sequences of antibodies, antibody fragments, receptors, immunoglobulin-like binding domains, peptides, aptimers, or can be completely synthetic. In some embodiments, the targeting moiety is non-proteinaceous; non-limiting examples of which are provided herein. The targeting moieties can comprise one or more functional antigen binding sites, the latter making the targeting moiety “multispecific.” An “antigen binding site” of a targeting moiety is one that is capable of binding a target antigen with at least a portion of the binding affinity of the parental antibody or receptor from which the antigen binding site is derived. The antigen binding site may itself be composed of more than one binding domain, linked together in the targeting moieties. “Binding domain” means a polypeptide sequence capable of attaching to an antigen or ligand but that may require additional binding domains to actually bind and/or sequester the antigen or ligand. A CDR from an antibody is an example of a binding domain. “Antibody” is used throughout the specification as a prototypical example of a targeting moiety (TM) but is not intended to be limiting.

Methods to measure binding affinity and/or other biologic activity of the targeted conjugate compositions of the invention can be those disclosed herein or methods generally known in the art. In addition, the physicochemical properties of the targeting moiety may be measured to ascertain the degree of target binding, solubility, structure and retention of stability. Assays are conducted that allow determination of binding characteristics of the targeting moieties towards a target, including binding dissociation constant (K_{d}, Kₒₙ and K_{off}), the half-life of dissociation of the ligand-receptor complex, as well as the activity of the targeting moiety to alter the biologic activity of the bound target compared to free target (IC₅₀ values). The term “K_{d}”, as used herein, is intended to refer to the dissociation constant of a particular antibody-antigen interaction as is known in the art, and would apply as a parameter of the binding affinity of a targeting moiety to its cognate ligand for the subject compositions. The term “Kₒₙ”, as used herein, is intended to refer to the on rate constant for association of an antibody to the antigen to form the antibody/antigen complex as is known in the art. The term “K_{off}”, as used herein, is intended to refer to the off rate constant for dissociation of an antibody from the antibody/antigen complex as is known in the art. The term “IC₅₀” refers to the concentration needed to inhibit half of the maximum biological response of the ligand agonist, and is generally determined by competition binding assays.

Techniques such as flow cytometry or surface plasmon resonance can be used to detect binding events. The assays may comprise soluble antigens or receptor molecules, or may determine the binding to cell-expressed receptors. Such assays may include cell-based assays, including assays for proliferation, cell death, apoptosis and cell migration. The binding affinity of the subject compositions for the target ligands can be assayed using binding or competitive binding assays, such as Biacore™ assays with chip-bound receptors or targeting moieties or ELISA assays, as described in U.S. Pat. No. 5,534,617, assays described in the Examples herein, radio-receptor assays, or other assays known in the art. The binding affinity constant can then be determined using standard methods, such as Scatchard analysis, as described by van Zoelen, et al., Trends Pharmacol Sciences (1998) 19)12):487, or other methods known in the art. In addition, libraries of sequence variants of targeting moieties can be compared to the corresponding native or parental antibodies using a competitive ELISA binding assay to determine whether they have the same binding specificity and affinity as the parental antibody, or some fraction thereof such that they are suitable for inclusion in the targeting moieties. The results of such assays can be used in an iterative process of sequence modification of the targeting moieties followed by binding and physicochemical characterization assays to guide the process by which specific constructs with the desired properties are selected.

The invention provides isolated targeting moieties in which the binding affinity of the one or more targeting moieties for target ligands can be at least about 1%, or at least about 10%, or at least about 20%, or at least about 30%, or at least about 40%, or at least about 50%, or at least about 60%, or at least about 70%, or at least about 80%, or at least about 90%, or at least about 95%, or at least about 99%, or at least about 99.9% or more of the affinity of a parental antibody or binding moiety not bound to XTEN for the target receptor or ligand. In one embodiment, the K_{d} between the one or more targeting moieties of the subject targeted conjugate composition and a target ligand or ligands is less than about 10⁻⁴ M, alternatively less than about 10⁻⁵ M, alternatively less than about 10⁻⁶ M, alternatively less than about 10⁻⁷ M, alternatively less than about 10⁻⁸ M, alternatively less than about 10⁻⁹ M, or less than about 10⁻¹⁰ M, or less than about 10⁻¹¹ M, or less than about 10⁻¹² M. In the foregoing embodiment, the binding affinity of the targeting moiety towards the target would be characterized as “specific.” The invention contemplates targeted conjugate compositions comprising two or more targeting moieties in which the binding affinities for the respective targeting moieties may independently be between the ranges of values of the foregoing. It will be understood by one of skill in the art that the TM component of the targeted conjugate compositions is intended to selectively or disproportionately deliver the composition and/or the payload(s) of the composition to the target tissue or cell, compared to healthy tissue or healthy cells in a subject in which the composition is administered or, in the case of in vitro assays, to the proximity of the target cells. In some of the foregoing embodiments f the paragraph, the one or more targeting moieties of the subject targeted conjugate compositions specifically bind to a target of Table 2, Table 3, Table 4, Table 18, or Table 19.

TABLE 18
Tumor cell lines

| | | | Receptor |
|---|---|---|---|
| Target | Cell line | Tissue | status |
| LHRHR | MCF-7 | Breast | positive |
| | MDA-MB-231 | Breast (HER2−/ER−/PR−) | positive |
| | HCC1806 | Breast (HER2−/ER−/PR−) | positive |
| | HCC1937 | Breast (HER2−/ER−/PR−) | positive |
| | OV-1063 | Ovarian | positive |
| | EFO-21 | Ovarian | positive |
| | EFO-27 | Ovarian | positive |
| | NIH:OVCAR-3 | Ovarian | positive |
| | BG-1 | Ovarian | positive |
| | HEC-1A | Endometrial | positive |
| | HEC-1B | Endometrial | positive |
| | Ishikawa | Endometrial | positive |
| | KLE | Endometrial | positive |
| | AN-3-CA | Endometrial | positive |
| | MiaPaCa | Pancreatic | positive |
| | Panc-1 | Pancreatic | positive |
| | rat Dunning | Prostate (androgen-dep) | positive |
| | R-3327-H | | |
| | PC-82 | Prostate (androgen-dep) | positive |
| | MDA-PCa-2b | Prostate (androgen-indep) | positive |
| | C4-2 (derivative | Prostate (androgen-dep) | positive |
| | of LNCaP) | | |
| | A549 | Lung | positive |
| | A2780 | Ovarian | positive |
| | UCI-107* | Ovarian | negative |
| | SK-OV-3* | Ovarian | negative |
| | SW 626 | Ovarian | negative |
| | MFE-296* | Endometrial | negative |
| Folate | KB | Nasopharyngeal | positive |
| receptor | IGROV | Ovarian | positive |
| | SK-OV-3 | Ovarian | positive |
| | HeLa | Cervical | positive |
| | LoVo | Colorectal | positive |
| | SW620 | Colorectal | positive |
| | MDA-MB-231 | Breast | positive |
| | Madison 109 | Lung | positive |
| | A549 | Lung | negative |
| | A375 | Multiple melanoma | negative |
| | LS-174T | Colorectal | negative |
| | SK-BR-3 | Breast | negative |
| | HT-29 | Colorectal | negative |
| | 4T1 | Breast | negative |
| | SK-BR-3 | Breast | negative |
| | PC-3 | Prostate | negative |
| Integrin | HUVEC | Endothelial | positive |
| | A2780 | Ovarian | positive |
| | OVCAR3 | Ovarian | positive |
| | H2009 | Lung | positive |
| | PC-3 | Prostate | positive |
| | DU145 | Prostate | positive |
| | MDA-MB-435 | Melanoma | positive |
| | HT29 | Colorectal | positive |
| | A549 | Lung | positive |
| | A498 | Kidney | positive |
| | Colo205 | Colorectal | positive |
| | U87MG | Glioblastoma | positive |
| | H1299 | Lung | negative |
| CD13 | A549 | Lung | positive |
| | MDA-MB-231 | Breast | negative |
| SSTR2 | HCC-1806 | Breast | positive |
| | A549 | Lung | positive |
| | HepG2 | Liver | positive |
| HER2 | SK-OV-3 | Ovarian | positive |
| | SK-Br-3 | Breast | positive |
| | MCF-7 | Breast | non-amplified |
| | BT-474 | Breast | positive |
| | HCC-1954 | Breast | positive |
| | A549 | Lung | positive |
| MUC-1 | HeLa | Cervical | positive |
| | OVCAR-3 | Ovarian | positive |
| | SK-OV-3 | Ovarian | positive |
| | LS-174T | Colorectal | negative |
| | MCF-7 | Breast | positive |
| | A549 | Lung | positive |
| PSMA | LNCaP | Prostate | positive |
| | MDA-PCa-2b | Prostate | positive |
| | CWR22Rv1 | Prostate | positive |
| | PC-3 | Prostate | negative |
| | DU145 | Prostate | negative |
| EpCAM | Colo205 | Colorectal | positive |
| | HCT-15 | Colorectal | positive |
| | WiDr | Colorectal | positive |
| | BxPc-3 | Pancreas | positive |
| | Capan-1 | Pancreas | positive |
| | OZ | Bile duct | positive |
| | MCF-7 | Breast | positive |
| | A549 | Lung | positive |
| | HepG2 | Liver | positive |
| | Hep3B | Liver | positive |
| | SK-Hep1 | Liver | negative |
| | Colo320DM | Colorectal | negative |
| EGFR | A549 | Lung | positive |
| | H292 | Lung | positive |
| | H1838 | Lung | positive |
| | SKMES | Lung | positive |
| | A431 | Epidermoid | positive |
| TAG-72 | LS174T | Colorectal | positive |
| | CWR22 | Prostate | positive |
| | HT29 | Colorectal | negative |

In one embodiment, the invention provides targeted conjugate compositions comprising targeting moieties capable of binding to a single target. In another embodiment, the targeting moieties of the invention are multispecific and the targeting moieties specifically bind at least two different target antigens or ligands (“bifunctional” or “multispecific”), or different epitopes on the same target. The multivalent targeting moieties can be designed to be bifunctional in that they can incorporate heterologous binding domains from different “parental” antibodies and bind two different ligands or antigens in order to better effect a desired pharmacological response; e.g., dimerization of receptors on a target cell surface leading to cell signaling or, alternatively, cell death, or modulating a biological function of one or more targets. Multispecific targeting moieties leading to cell death, whether by triggering apoptosis or necrosis or by the effects of the delivered cytotoxic payload, are expected to have utility in, particularly, the treatment of oncological disease. Non-limiting examples of pairs of targets contemplated as suitable for multivalent, bifunctional targeting moieties include: IGF1 and IGF2; IGF1/2 and Erb2B; VEGFR and EGFR, CD20 and CD3, CD138 and CD20, CD38 and CD20, CD38 & CD138, CD40 and CD20, CD138 and CD40, CD38 and CD40, IL-1α and IL-1β, IL-12 and IL-18, TNFα and IL-23, TNFα and IL-13, TNF and IL-18, TNF and IL-12, TNF and IL-1beta, TNF and MIF, TNF and IL-17, and TNF and IL-15, TNF and VEGF, VEGFR and EGFR, IL-13 and IL-9, IL-13 and IL-4, IL-13 and IL-5, IL-13 and IL-25, IL-13 and TARC, IL-13 and MDC, IL-13 and MIF, IL-13 and TGF-β, IL-13 and LHR agonist, IL-13 and CL25, IL-13 and SPRR2a, IL-13 and SPRR2b, IL-13 and ADAM8, and TNFα and PGE4, IL-13 and PED2, TNF and PEG2, CD19 and CD20, CD-8 and IL-6, PDL-1 and CTLA-4, CTLA-4 and BTNO2, CSPGs and RGM A, IL-12 and IL-18, IL-12 and TWEAK, IL-13 and ADAM8, IL-13 and CL25, IL-13 and IL-1beta, IL-13 and IL-25, IL-13 and IL-4, IL-13 and IL-5, IL-13 and IL-9, IL-13 and LHR agonist, IL-13 and MDC, IL-13 and MIF, IL-13 and PED2, IL-13 and SPRR2a, IL-13 and SPRR2b, IL-13 and TARC, IL-13 and TGF-β, IL-1α and IL-1β, MAG and RGM A, NgR and RGM A, NogoA and RGM A, OMGp and RGM A, RGM A and RGM B, Te38 and TNFα, TNFα and IL-12, TNFα and IL-12p40, TNFα and IL-13, TNFα and IL-15, TNFα and IL-17, TNFα and IL-18, TNFα and IL-1beta, TNFα and IL-23, TNFα and MIF, TNFα and PEG2, TNFα and PGE4, TNFα and VEGF, TNFα and RANK ligand, TNFα and Blys, TNFα and GP130, TNFα and CD-22; and TNFα and CTLA-4,

The targeting moieties of the targeted conjugate composition can be derived from one or more fragments of various monoclonal antibodies known in the art. Non-limiting examples of such monoclonal antibodies include, but are not limited to anti-TNF antibody (U.S. Pat. No. 6,258,562), anti-IL-12 and or anti-IL-12p40 antibody (U.S. Pat. No. 6,914,128); anti-IL-18 antibody (US 2005/0147610 A1), anti-RANKL (U.S. Pat. No. 7,411,050), anti-C5, anti-CBL, anti-CD147, anti-gp120, anti-VLA4, anti-CD11a, anti-CD18, anti-VEGF, anti-CD40L, anti-Id, anti-ICAM-1, anti-CXCL13, anti-CD2, anti-EGFR, anti-TGF-beta 2, anti-E-selectin, anti-Fact VII, anti-Her2/neu, anti-Fgp, anti-CD11/18, anti-CD14, anti-ICAM-3, anti-CD80, anti-CD4, anti-CD3, anti-CD23, anti-beta2-integrin, anti-alpha4beta7, anti-CD52, anti-HLA DR, anti-CD22, anti-CD20, anti-MIF, anti-CD64 (FcR), anti-TCR alpha beta, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-VNRintegrin, anti-IL-1alpha, anti-IL-1beta, anti-IL-1 receptor, anti-IL-2 receptor, anti-IL-4, anti-IL4 receptor, anti-ILS, anti-IL-5 receptor, anti-IL-6, anti-IL-8, anti-IL-9, anti-IL-13, anti-IL-13 receptor, anti-IL-17, and anti-IL-23 (see Presta L G. 2005 Selection, design, and engineering of therapeutic antibodies J Allergy Clin Immunol. 116:731-6 and Clark, M., “Antibodies for Therapeutic Applications,” Department of Pathology, Cambridge University, UK, 15 Oct. 2000, published online at M. Clark's home page at the website for the Department of Pathology, Cambridge University).

In some embodiments, the targeting moieties are derived from one or more fragments of therapeutic monoclonal antibodies approved for use in humans or antibodies that have demonstrated efficacy in clinical trials or established preclinical models of diseases, disorders or conditions. Non-limiting examples of such monoclonal antibodies are presented in Table 19. Such therapeutic antibodies include, but are not limited to, rituximab, IDEC/Genentech/Roche (see for example U.S. Pat. No. 5,736,137), a chimeric anti-CD20 antibody used in the treatment of many lymphomas, leukemias, and some autoimmune disorders; ofatumumab, an anti-CD20 antibody approved for use for chronic lymphocytic leukemia, and under development for follicular non-Hodgkin's lymphoma, diffuse large B cell lymphoma, rheumatoid arthritis and relapsing remitting multiple sclerosis, being developed by GlaxoSmithKline; lucatumumab (HCD122), an anti-CD40 antibody developed by Novartis for Non-Hodgkin's or Hodgkin's Lymphoma (see, for example, U.S. Pat. No. 6,899,879), AME-133, an antibody developed by Applied Molecular Evolution which binds to cells expressing CD20 to treat non-Hodgkin's lymphoma, veltuzumab (hA20), an antibody developed by Immunomedics, Inc. which binds to cells expressing CD20 to treat immune thrombocytopenic purpura, HumaLYM developed by Intracel for the treatment of low-grade B-cell lymphoma, and ocrelizumab, developed by Genentech which is an anti-CD20 monoclonal antibody for treatment of rheumatoid arthritis (see for example U.S. Patent Application 20090155257), trastuzumab (see for example U.S. Pat. No. 5,677,171), a humanized anti-Her2/neu antibody approved to treat breast cancer developed by Genentech; pertuzumab, an anti-Her2 dimerization inhibitor antibody developed by Genentech in treatment of in prostate, breast, and ovarian cancers; (see for example U.S. Pat. No. 4,753,894); cetuximab, an anti-EGRF antibody used to treat epidermal growth factor receptor (EGFR)-expressing, KRAS wild-type metastatic colorectal cancer and head and neck cancer, developed by Imclone and BMS (see U.S. Pat. No. 4,943,533; PCT WO 96/40210); panitumumab, a fully human monoclonal antibody specific to the epidermal growth factor receptor (also known as EGF receptor, EGFR, ErbB-1 and Herl, currently marketed by Amgen for treatment of metastatic colorectal cancer (see U.S. Pat. No. 6,235,883); zalutumumab, a fully human IgG1 monoclonal antibody developed by Genmab that is directed towards the epidermal growth factor receptor (EGFR) for the treatment of squamous cell carcinoma of the head and neck (see for example U.S. Pat. No. 7,247,301); nimotuzumab, a chimeric antibody to EGFR developed by Biocon, YM Biosciences, Cuba, and Oncosciences, Europe) in the treatment of squamous cell carcinomas of the head and neck, nasopharyngeal cancer and glioma (see for example U.S. Pat. No. 5,891,996; U.S. Pat. No. 6,506,883); alemtuzumab, a humanized monoclonal antibody to CD52 marketed by Bayer Schering Pharma for the treatment of chronic lymphocytic leukemia (CLL), cutaneous T-cell lymphoma (CTCL) and T-cell lymphoma; muromonab-CD3, an anti-CD3 antibody developed by Ortho Biotech/Johnson & Johnson used as an immunosuppressant biologic given to reduce acute rejection in patients with organ transplants; ibritumomab tiuxetan, an anti-CD20 monoclonal antibody developed by IDEC/Schering AG as treatment for some forms of B cell non-Hodgkin's lymphoma; gemtuzumab ozogamicin, an anti-CD33 (p67 protein) antibody linked to a cytotoxic chelator tiuxetan, to which a radioactive isotope is attached, developed by Celltech/Wyeth used to treat acute myelogenous leukemia; alefacept, an anti-LFA-3 Fc fusion developed by Biogen that is used to control inflammation in moderate to severe psoriasis with plaque formation; abciximab, made from the Fab fragments of an antibody to the IIb/IIIa receptor on the platelet membrane developed by Centocor/Lilly as a platelet aggregation inhibitor mainly used during and after coronary artery procedures; basiliximab, a chimeric mouse-human monoclonal antibody to the a chain (CD25) of the IL-2 receptor of T cells, developed by Novartis, used to prevent rejection in organ transplantation; palivizumab, developed by Medimmune; infliximab (REMICADE), an anti-TNFalpha antibody developed by Centocor/Johnson and Johnson, adalimumab (HUMIRA), an anti-TNFalpha antibody developed by Abbott, HUMICADE, an anti-TNFalpha antibody developed by Celltech, etanercept (ENBREL), an anti-TNFalpha Fc fusion developed by Immunex/Amgen, ABX-CBL, an anti-CD147 antibody developed by Abgenix, ABX-IL8, an anti-IL8 antibody developed by Abgenix, ABX-MA1, an anti-MUC18 antibody developed by Abgenix, Pemtumomab (R1549, 90Y-muHMFG1), an anti-MUC1 in development by Antisoma, Therex (R1550), an anti-MUC1 antibody developed by Antisoma, AngioMab (AS1405), developed by Antisoma, HuBC-1, developed by Antisoma, Thioplatin (AS1407) developed by Antisoma, ANTEGREN (natalizumab) a humanized monoclonal antibody against the cell adhesion molecule a4-integrin, an anti-alpha-4-beta-1 (VLA4) and alpha-4-beta-7 antibody developed by Biogen, VLA-1 mAb, an anti-VLA-1 integrin antibody developed by Biogen, LTBR mAb, an anti-lymphotoxin beta receptor (LTBR) antibody developed by Biogen, CAT-152, an anti-TGF-β2 antibody developed by Cambridge Antibody Technology, J695, an anti-IL-12 antibody developed by Cambridge Antibody Technology and Abbott, CAT-192, an anti-TGFβ1 antibody developed by Cambridge Antibody Technology and Genzyme, CAT-213, an anti-Eotaxin1 antibody developed by Cambridge Antibody Technology, LYMPHOSTAT-B, an anti-Blys antibody developed by Cambridge Antibody Technology and Human Genome Sciences Inc., TRAIL-R1mAb, an anti-TRAIL-R1 antibody developed by Cambridge Antibody Technology and Human Genome Sciences, Inc., bevacizumab (AVASTIN, rhuMAb-VEGF), an anti-VEGF antibody developed by Genentech, HERCEPTIN, an anti-HER receptor family antibody developed by Genentech, Anti-Tissue Factor (ATF), an anti-Tissue Factor antibody developed by Genentech, XOLAIR (Omalizumab), an anti-IgE antibody developed by Genentech, MLN-02 Antibody (formerly LDP-02), developed by Genentech and Millennium Pharmaceuticals, HUMAX CD4®, an anti-CD4 antibody developed by Genmab, tocilizuma, and anti-IL6R antibody developed by Chugai, HUMAX-IL15, an anti-IL15 antibody developed by Genmab and Amgen, HUMAX-Inflam, developed by Genmab and Medarex, HUMAX-Cancer, an anti-Heparanase I antibody developed by Genmab and Medarex and Oxford GlycoSciences, HUMAX-Lymphoma, developed by Genmab and Amgen, HUMAX-TAC, developed by Genmab, IDEC-131, and anti-CD40L antibody developed by IDEC Pharmaceuticals, IDEC-151 (Clenoliximab), an anti-CD4 antibody developed by IDEC Pharmaceuticals, IDEC-114, an anti-CD80 antibody developed by IDEC Pharmaceuticals, IDEC-152, an anti-CD23 developed by IDEC Pharmaceuticals, anti-macrophage migration factor (MIF) antibodies developed by IDEC Pharmaceuticals, BEC2, an anti-idiotypic antibody developed by Imclone, IMC-1C11, an anti-KDR antibody developed by Imclone, DC101, an anti-flk-1 antibody developed by Imclone, anti-VE cadherin antibodies developed by Imclone, CEA-CIDE (labetuzumab), an anti-carcinoembryonic antigen (CEA) antibody developed by Immunomedics, Yervoy (ipilimumab), an anti-CTLA4 antibody developed by Bristol-Myers Sequibb in the treatment of melanoma, Lymphocide® (Epratuzumab) an anti-CD22 antibody developed by Immunomedics, AFP-Cide, developed by Immunomedics, MyelomaCide, developed by Immunomedics, LkoCide, developed by Immunomedics, ProstaCide, developed by Immunomedics, MDX-010, an anti-CTLA4 antibody developed by Medarex, MDX-060, an anti-CD30 antibody developed by Medarex, MDX-070 developed by Medarex, MDX-018 developed by Medarex, OSIDEM (IDM-1), and anti-Her2 antibody developed by Medarex and Immuno-Designed Molecules, HUMAX®-CD4, an anti-CD4 antibody developed by Medarex and Genmab, HuMax-IL15, an anti-IL15 antibody developed by Medarex and Genmab, CNTO 148, an anti-TNFa antibody developed by Medarex and Centocor/J&J, CNTO 1275, an anti-cytokine antibody developed by Centocor/J&J, MOR101 and MOR102, anti-intercellular adhesion molecule-1 (ICAM-1) (CD54) antibodies developed by MorphoSys, MOR201, an anti-fibroblast growth factor receptor 3 (FGFR-3) antibody developed by MorphoSys, tremelimumab, an anti-CTLA-4 antibody developed by Pfizer, visilizumab, an anti-CD3 antibody developed by Protein Design Labs, HUZAF, an anti-gamma interferon antibody developed by Protein Design Labs, Anti-a 5β1 Integrin, developed by Protein Design Labs, anti-IL-12, developed by Protein Design Labs, ING-1, an anti-Ep-CAM antibody developed by Xoma, XOLAIR® (Omalizumab) a humanized anti-IgE antibody developed by Genentech and Novartis, and MLN01, an anti-Beta2 integrin antibody developed by Xoma; all of the above-cited antibody references in this paragraph are expressly incorporated herein by reference. The sequences for the above antibodies can be obtained from publicly available databases, patents, or literature references. In addition, non-limiting examples of VH and VL sequences from such monoclonal antibody sequences are presented in Table 19. Examplary linkers suitable for recombinantly linking the VL and VH sequences as scFv or other such antibody fragment compositions are presented in Table 19, but the invention also contemplates use of linkers known in the art for the generation of scFv.

TABLE 19
Monoclonal Antibodies and Sequences

| Trade | Antibody | | | SEQ ID | | SEQ ID |
|---|---|---|---|---|---|---|
| Name | Name | Target | VH Sequence | NO: | VL Sequence | NO: |
| Tysabri ™ | natalizumab | Alpha 4 | QVQLVQSGAEVKK | 432 | DIQMTQSPSSLSAS | 511 |
| | | Integrin | PGASVKVSCKASG | | VGDRVTITCKTSQD | |
| | | | FNIKDTYIHWVRQ | | INKYMAWYQQTPGK | |
| | | | APGQRLEWMGRID | | APRLLIHYTSALQP | |
| | | | PANGYTKYDPKFQ | | GIPSRFSGSGSGRD | |
| | | | GRVTITADTSAST | | YTFTISSLQPEDIA | |
| | | | AYMELSSLRSEDT | | TYYCLQYDNLWTFG | |
| | | | AVYYCAREGYYGN | | QGTKVEIK | |
| | | | YGVYAMDYWGQGT | | | |
| | | | LVTVSS | | | |
| REGN910 | nesvacumab | Ang2 | EVQLVESGGGLVQ | 433 | EIVLTQSPGTLSLS | 512 |
| | | | PGGSLRLSCAASG | | PGERATLSCRASQS | |
| | | | FTESSYDIHWVRQ | | VSSTYLAWYQQKPG | |
| | | | ATGKGLEWVSAIG | | QAPRLLIYGASSRA | |
| | | | PAGDTYYPGSVKG | | TGIPDRFSGSGSGT | |
| | | | RFTISRENAKNSL | | DFTLTISRLEPEDF | |
| | | | YLQMNSLRAGDTA | | AVYYCQHYDNSQTF | |
| | | | VYYCARGLITFGG | | GQGTKVEIK | |
| | | | LIAPFDYWGQGTL | | | |
| | | | VTVSS | | | |
| hMFE23 | | CEA | QVKLEQSGAEVVK | 434 | ENVLTQSPSSMSAS | 513 |
| | | | PGASVKLSCKASG | | VGDRVNIACSASSS | |
| | | | FNIKDSYMHWLRQ | | VSYMHWFQQKPGKS | |
| | | | GPGQRLEWIGWID | | PKLWIYSTSNLASG | |
| | | | PENGDTEYAPKFQ | | VPSRFSGSGSGTDY | |
| | | | GKATFTTDTSANT | | SLTISSMQPEDAAT | |
| | | | AYLGLSSLRPEDT | | YYCQQRSSYPLTFG | |
| | | | AVYYCNEGTPTGP | | GGTKLEIK | |
| | | | YYFDYWGQGTLVT | | | |
| | | | VSS | | | |
| M5A | | CEA | EVQLVESGGGLVQ | 435 | DIQLTQSPSSLSAS | 514 |
| (humanized | | | PGGSLRLSCAASG | | VGDRVTITCRAGES | |
| T84.66) | | | FNIKDTYMHWVRQ | | VDIFGVGFLHWYQQ | |
| | | | APGKGLEWVARID | | KPGKAPKLLIYRAS | |
| | | | PANGNSKYADSVK | | NLESGVPSRFSGSG | |
| | | | GRFTISADTSKNT | | SRTDFTLTISSLQP | |
| | | | AYLQMNSLRAEDT | | EDFATYYCQQTNED | |
| | | | AVYYCAPFGYYVS | | PYTFGQGTKVEIK | |
| | | | DYAMAYWGQGTLV | | | |
| | | | TVSS | | | |
| M5B | | CEA | EVQLVESGGGLVQ | 436 | DIQLTQSPSSLSAS | 515 |
| (humanized | | | PGGSLRLSCAASG | | VGDRVTITCRAGES | |
| T84.66) | | | FNIKDTYMHWVRQ | | VDIFGVGFLHWYQQ | |
| | | | APGKGLEWVARID | | KPGKAPKLLIYRAS | |
| | | | PANGNSKYVPKFQ | | NLESGVPSRFSGSG | |
| | | | GRATISADTSKNT | | SRTDFTLTISSLQP | |
| | | | AYLQMNSLRAEDT | | EDFATYYCQQTNED | |
| | | | AVYYCAPFGYYVS | | PYTFGQGTKVEIK | |
| | | | DYAMAYWGQGTLV | | | |
| | | | TVSS | | | |
| CEA-Cide | Labetuzumab | CEACAM5 | EVQLVESGGGVVQ | 437 | DIQLTQSPSSLSAS | 516 |
| | (MN-14) | | PGRSLRLSCSASG | | VGDRVTITCKASQD | |
| | | | FDFTTYWMSWVRQ | | VGTSVAWYQQKPGK | |
| | | | APGKGLEWIGEIH | | APKLLIYWTSTRHT | |
| | | | PDSSTINYAPSLK | | GVPSRFSGSGSGTD | |
| | | | DRFTISRDNAKNT | | FTFTISSLQPEDIA | |
| | | | LFLQMDSLRPEDT | | TYYCQQYSLYRSFG | |
| | | | GVYFCASLYFGFP | | QGTKVEIK | |
| | | | WFAYWGQGTPVTV | | | |
| | | | SS | | | |
| CEA-Scan | arcitumomab | CEACAM5 | EVKLVESGGGLVQ | 438 | QTVLSQSPAILSAS | 517 |
| | | | PGGSLRLSCATSG | | PGEKVTMTCRASSS | |
| | | | FTFTDYYMNWVRQ | | VTYIHWYQQKPGSS | |
| | | | PPGKALEWLGFIG | | PKSWIYATSNLASG | |
| | | | NKANGYTTEYSAS | | VPARFSGSGSGTSY | |
| | | | VKGRFTISRDKSQ | | SLTISRVEAEDAAT | |
| | | | SILYLQMNTLRAE | | YYCQHWSSKPPTFG | |
| | | | DSATYYCTRDRGL | | GGTKLEIKR | |
| | | | RFYFDYWGQGTTL | | | |
| | | | TVSS | | | |
| MT111 | | CEACAM5 | EVQLVESGGGLVQ | 439 | QAVLTQPASLSASP | 518 |
| | | | PGRSLRLSCAASG | | GASASLTCTLRRGI | |
| | | | FTVSSYWMHWVRQ | | NVGAYSIYWYQQKP | |
| | | | APGKGLEWVGFIR | | GSPPQYLLRYKSDS | |
| | | | NKANGGTTEYAAS | | DKQQGSGVSSRFSA | |
| | | | VKGRFTISRDDSK | | SKDASANAGILLIS | |
| | | | NTLYLQMNSLRAE | | GLQSEDEADYYCMI | |
| | | | DTAVYYCARDRGL | | WHSGASAVFGGGTK | |
| | | | RFYFDYWGQGTTV | | LTVL | |
| | | | TVSS | | | |
| MT103 | blinatumomab | CD19 | QVQLQQSGAELVR | 440 | DIQLTQSPASLAVS | 519 |
| | | | PGSSVKISCKASG | | LGQRATISCKASQS | |
| | | | YAFSSYWMNWVKQ | | VDYDGDSYLNWYQQ | |
| | | | RPGQGLEWIGQIW | | IPGQPPKLLIYDAS | |
| | | | PGDGDTNYNGKFK | | NLVSGIPPRFSGSG | |
| | | | GKATLTADESSST | | SGTDFTLNIHPVEK | |
| | | | AYMQLSSLASEDS | | VDAATYHCQQSTED | |
| | | | AVYFCARRETTTV | | PWTFGGGTKLEIK | |
| | | | GRYYYAMDYWGQG | | | |
| | | | TTVTVSS | | | |
| Arzerra | ofatumumab | CD20 | EVQLVESGGGLVQ | 441 | EIVLTQSPATLSLS | 520 |
| | | | PGRSLRLSCAASG | | PGERATLSCRASQS | |
| | | | FTFNDYAMHWVRQ | | VSSYLAWYQQKPGQ | |
| | | | APGKGLEWVSTIS | | APRLLIYDASNRAT | |
| | | | WNSGSIGYADSVK | | GIPARFSGSGSGTD | |
| | | | GRFTISRDNAKKS | | FTLTISSLEPEDFA | |
| | | | LYLQMNSLRAEDT | | VYYCQQRSNWPITF | |
| | | | ALYYCAKDIQYGN | | GQGTRLEIKR | |
| | | | YYYGMDVWGQGTT | | | |
| | | | VTVSSA | | | |
| Bexxar ™ | toSitumomab | CD20 | QAYLQQSGAELVR | 442 | QIVLSQSPAILSAS | 521 |
| | | | PGASVKMSCKASG | | PGEKVTMTCRASSS | |
| | | | YTFTSYNMHWVKQ | | VSYMHWYQQKPGSS | |
| | | | TPRQGLEWIGAIY | | PKPWIYAPSNLASG | |
| | | | PGNGDTSYNQKFK | | VPARFSGSGSGTSY | |
| | | | GKATLTVDKSSST | | SLTISRVEAEDAAT | |
| | | | AYMQLSSLTSEDS | | YYCQQWSFNPPTFG | |
| | | | AVYFCARVVYYSN | | AGTKLELK | |
| | | | SYWYFDVWGTGTT | | | |
| | | | VTVSG | | | |
| GAZYVA | Obinutuzumab | CD20 | QVQLVQSGAEVKK | 443 | DIVMTQTPLSLPVT | 522 |
| | | | PGSSVKVSCKASG | | PGEPASISCRSSKS | |
| | | | YAFSYSWINWVRQ | | LLHSNGITYLYWYL | |
| | | | APGQGLEWMGRIF | | QKPGQSPQLLIYQM | |
| | | | PGDGDTDYNGKFK | | SNLVSGVPDRFSGS | |
| | | | GRVTITADKSTST | | GSGTDFTLKISRVE | |
| | | | AYMELSSLRSEDT | | AEDVGVYYCAQNLE | |
| | | | AVYYCARNVFDGY | | LPYTEGGGTKVEIK | |
| | | | WLVYWGQGTLVTV | | | |
| | | | SS | | | |
| | Ocrelizumab/ | CD20 | EVQLVESGGGLVQ | 444 | DIQMTQSPSSLSAS | 523 |
| | 2H7 v16 | | PGGSLRLSCAASG | | VGDRVTITCRASSS | |
| | | | YTFTSYNMHWVRQ | | VSYMHWYQQKPGKA | |
| | | | APGKGLEWVGAIY | | PKPLIYAPSNLASG | |
| | | | PGNGDTSYNQKFK | | VPSRFSGSGSGTDF | |
| | | | GRFTISVDKSKNT | | TLTISSLQPEDFAT | |
| | | | LYLQMNSLRAEDT | | YYCQQWSFNPPTFG | |
| | | | AVYYCARVVYYSN | | QGTKVEIKR | |
| | | | SYWYFDVWGQGTL | | | |
| | | | VTVSS | | | |
| Rituxan ™ | rituximab | CD20 | QVQLQQPGAELVK | 445 | QIVLSQSPAILSAS | 524 |
| | | | PGASVKMSCKASG | | PGEKVTMTCRASSS | |
| | | | YTFTSYNMHWVKQ | | VSYTHWFQQKPGSS | |
| | | | TPGRGLEWIGAIY | | PKPWIYATSNLASG | |
| | | | PGNGDTSYNQKFK | | VPVRFSGSGSGTSY | |
| | | | GKATLTADKSSST | | SLTISRVEAEDAAT | |
| | | | AYMQLSSLTSEDS | | YYCQQWTSNPPTFG | |
| | | | AVYYCARSTYYGG | | GGTKLEIKR | |
| | | | DWYFNVWGAGTTV | | | |
| | | | TVSAA | | | |
| Zevalin ™ | ibritumomab | CD20 | QAYLQQSGAELVR | 446 | QIVLSQSPAILSAS | 525 |
| | tieuxetan | | PGASVKMSCKASG | | PGEKVTMTCRASSS | |
| | | | YTFTSYNMHWVKQ | | VSYMHWYQQKPGSS | |
| | | | TPRQGLEWIGAIY | | PKPWIYAPSNLASG | |
| | | | PGNGDTSYNQKFK | | VPARFSGSGSGTSY | |
| | | | GKATLTVDKSSST | | SLTISRVEAEDAAT | |
| | | | AYMQLSSLTSEDS | | YYCQQWSFNPPTFG | |
| | | | AVYFCARVVYYSN | | AGTKLELK | |
| | | | SYWYFDVWGTGTT | | | |
| | | | VTVSA | | | |
| Mylotarg | gemtuzumab | CD33 | QLVQSGAEVKKPG | 447 | DIQLTQSPSTLSAS | 526 |
| | | | SSVKVSCKASGYT | | VGDRVTITCRASES | |
| | | | ITDSNIHWVRQAP | | LDNYGIRFLTWFQQ | |
| | | | GQSLEWIGYIYPY | | KPGKAPKLLMYAAS | |
| | | | NGGTDYNQKFKNR | | NQGSGVPSRFSGSG | |
| | | | ATLTVDNPTNTAY | | SGTEFTLTISSLQP | |
| | | | MELSSLRSEDTDF | | DDFATYYCQQTKEV | |
| | | | YYCVNGNPWLAYW | | PWSFGQGTKVEVKR | |
| | | | GQGTLVTVSSAST | | T | |
| | | | KGP | | | |
| Daratumumab | | CD38 | EVQLLESGGGLVQ | 448 | EIVLTQSPATLSLS | 527 |
| | | | PGGSLRLSCAVSG | | PGERATLSCRASQS | |
| | | | FTFNSFAMSWVRQ | | VSSYLAWYQQKPGQ | |
| | | | APGKGLEWVSAIS | | APRLLIYDASNRAT | |
| | | | GSGGGTYYADSVK | | GIPARFSGSGSGTD | |
| | | | GRFTISRDNSKNT | | FTLTISSLEPEDFA | |
| | | | LYLQMNSLRAEDT | | VYYCQQRSNWPPTF | |
| | | | AVYFCAKDKILWF | | GQGTKVEIK | |
| | | | GEPVFDYWGQGTL | | | |
| | | | VTVSS | | | |
| CE-355621 | | cMET | QVQLVQSGAEVKK | 449 | DIQMTQSPSSVSAS | 528 |
| | | | PGASVKVSCKASG | | VGDRVTITCRASQG | |
| | | | YTFTSYGFSWVRQ | | INTWLAWYQQKPGK | |
| | | | APGQGLEWMGWIS | | APKLLIYAASSLKS | |
| | | | ASNGNTYYAQKLQ | | GVPSRFSGSGSGTD | |
| | | | GRVTMTTDTSTST | | FTLTISSLQPEDFA | |
| | | | AYMELRSLRSDDT | | TYYCQQANSFPLTF | |
| | | | AVYYCARVYADYA | | GGGTKVEIK | |
| | | | DYWGQGTLVTVSS | | | |
| LY2875358 | emibetuzumab | cMET | QVQLVQSGAEVKK | 450 | DIQMTQSPSSLSAS | 529 |
| | | | PGASVKVSCKASG | | VGDRVTITCSVSSS | |
| | | | YTFTDYYMHWVRQ | | VSSIYLHWYQQKPG | |
| | | | APGQGLEWMGRVN | | KAPKLLIYSTSNLA | |
| | | | PNRRGTTYNQKFE | | SGVPSRFSGSGSGT | |
| | | | GRVTMTTDTSTST | | DFTLTISSLQPEDF | |
| | | | AYMELRSLRSDDT | | ATYYCQVYSGYPLT | |
| | | | AVYYCARANWLDY | | FGGGTKVEIK | |
| | | | WGQGTTVTVSS | | | |
| MetMAb | onartuzumab | cMET | EVQLVESGGGLVQ | 451 | DIQMTQSPSSLSAS | 530 |
| | | | PGGSLRLSCAASG | | VGDRVTITCKSSQS | |
| | | | YTFTSYWLHWVRQ | | LLYTSSQKNYLAWY | |
| | | | APGKGLEWVGMID | | QQKPGKAPKLLIYW | |
| | | | PSNSDTRFNPNFK | | ASTRESGVPSRFSG | |
| | | | DRFTISADTSKNT | | SGSGTDFTLTISSL | |
| | | | AYLQMNSLRAEDT | | QPEDFATYYCQQYY | |
| | | | AVYYCATYRSYVT | | AYPWTFGQGTKVEI | |
| | | | PLDYWGQGTLVTV | | KR | |
| | | | SSA | | | |
| | tremelimumab | CTLA4 | QVQLVESGGGVVQ | 452 | DIQMTQSPSSLSAS | 531 |
| | (CP-675206, | | PGRSLRLSCAASG | | VGDRVTITCRASQS | |
| | or 11.2.1) | | FTFSSYGMHWVRQ | | INSYLDWYQQKPGK | |
| | | | APGKGLEWVAVIW | | APKLLIYAASSLQS | |
| | | | YDGSNKYYADSVK | | GVPSRFSGSGSGTD | |
| | | | GRFTISRDNSKNT | | FTLTISSLQPEDFA | |
| | | | LYLQMNSLRAEDT | | TYYCQQYYSTPFTF | |
| | | | AVYYCARDPRGAT | | GPGTKVEIKR | |
| | | | LYYYYYGMDVWGQ | | | |
| | | | GTTVTVSS | | | |
| Yervoy | Ipilimumab | CTLA4 | QVQLVESGGGVVQ | 453 | EIVLTQSPGTLSLS | 532 |
| | 10D1 | | PGRSLRLSCAASG | | PGERATLSCRASQS | |
| | | | FTFSSYTMHWVRQ | | VGSSYLAWYQQKPG | |
| | | | APGKGLEWVTFIS | | QAPRLLIYGAFSRA | |
| | | | YDGNNKYYADSVK | | TGIPDRFSGSGSGT | |
| | | | GRFTISRDNSKNT | | DFTLTISRLEPEDF | |
| | | | LYLQMNSLRAEDT | | AVYYCQQYGSSPWT | |
| | | | AIYYCARTGWLGP | | FGQGTKVEIK | |
| | | | FDYWGQGTLVTVS | | | |
| | | | S | | | |
| MT110 | solitomab | EpCAM | EVQLLEQSGAELV | 454 | ELVMTQSPSSLTVT | 533 |
| | | | RPGTSVKISCKAS | | AGEKVTMSCKSSQS | |
| | | | GYAFTNYWLGWVK | | LLNSGNQKNYLTWY | |
| | | | QRPGHGLEWIGDI | | QQKPGQPPKLLIYW | |
| | | | FPGSGNIHYNEKF | | ASTRESGVPDRFTG | |
| | | | KGKATLTADKSSS | | SGSGTDFTLTISSV | |
| | | | TAYMQLSSLTFED | | QAEDLAVYYCQNDY | |
| | | | SAVYFCARLRNWD | | SYPLTFGAGTKLEI | |
| | | | EPMDYWGQGTTVT | | K | |
| | | | VSS | | | |
| MT201 | Adecatumumab | EpCAM | EVQLLESGGGVVQ | 455 | ELQMTQSPSSLSAS | 534 |
| | | | PGRSLRLSCAASG | | VGDRVTITCRTSQS | |
| | | | FTFSSYGMHWVRQ | | ISSYLNWYQQKPGQ | |
| | | | APGKGLEWVAVIS | | PPKLLIYWATRES | |
| | | | YDGSNKYYADSVK | | GVPDRESGSGSGTD | |
| | | | GRFTISRDNSKNT | | FTLTISSLQPEDSA | |
| | | | LYLQMNSLRAEDT | | TYYCOQSYDIPYTF | |
| | | | AVYYCAKDMGWGS | | GQGTKLEIK | |
| | | | GWRPYYYYGMDVW | | | |
| | | | GQGTTVTVSS | | | |
| Panorex | Edrecolomab | EpCAM | QVQLQQSGAELVR | 456 | NIVMTQSPKSMSMS | 535 |
| | Mab CO17- | | PGTSVKVSCKASG | | VGERVTLTCKASEN | |
| | 1A | | YAFTNYLIEWVKQ | | VVTYVSWYQQKPEQ | |
| | | | RPGQGLEWIGVIN | | SPKLLIYGASNRYT | |
| | | | PGSGGTNYNEKFK | | GVPDRFTGSGSATD | |
| | | | GKATLTADKSSST | | FTLTISSVQAEDLA | |
| | | | AYMQLSSLTSDDS | | DYHCGQGYSYPYTF | |
| | | | AVYFCARDGPWFA | | GGGTKLEIK | |
| | | | YWGQGTLVTVSA | | | |
| | tucotuzumab | EpCAM | QIQLVQSGPELKK | 457 | QILLTQSPAIMSAS | 536 |
| | | | PGETVKISCKASG | | PGEKVTMTCSASSS | |
| | | | YTFTNYGMNWVRQ | | VSYMLWYQQKPGSS | |
| | | | APGKGLKWMGWIN | | PKPWIFDTSNLASG | |
| | | | TYTGEPTYADDFK | | FPARFSGSGSGTSY | |
| | | | GRFVFSLETSAST | | SLIISSMEAEDAAT | |
| | | | AFLQLNNLRSEDT | | YYCHQRSGYPYTFG | |
| | | | ATYFCVRFISKGD | | GGTKLEIK | |
| | | | YWGQGTSVTVSS | | | |
| UBS-54 | | EpCAM | VQLQQSDAELVKP | 458 | DIVMTQSPDSLAVS | 537 |
| | | | GASVKISCKASGY | | LGERATINCKSSQS | |
| | | | TFTDHAIHWVKQN | | VLYSSNNKNYLAWY | |
| | | | PEQGLEWIGYFSP | | QQKPGQPPKLLIYW | |
| | | | GNDDFKYNERFKG | | ASTRESGVPDRFSG | |
| | | | KATLTADKSSSTA | | SGSGTDFTLTISSL | |
| | | | YVQLNSLTSEDSA | | QAEDVAVYYCQQYY | |
| | | | VYFCTRSLNMAYW | | SYPLTFGGGTKVKE | |
| | | | GQGTSVTVS | | SGSVSS | |
| 3622W94 | | EpCAM | EVQLVQSGPEVKK | 459 | DIVMTQSPLSLPVT | 538 |
| | | | PGASVKVSCKASG | | PGEPASISCRSSIN | |
| | | | YTFTNYGMNWVRQ | | KKGSNGITYLYWYL | |
| | | | APGQGLEWMGWIN | | QKPGQSPQLLIYQM | |
| | | | TYTGEPTYGEDFK | | SNLASGVPDRFSGS | |
| | | | GRFAFSLDTSAST | | GSGTDFTLKISRVE | |
| | | | AYMELSSLRSEDT | | AEDVGVYYCAQNLE | |
| | | | AVYFCARFGNYVD | | IPRTFGQGTKVEIK | |
| | | | YWGQGSLVTVSS | | R | |
| 4D5MOCB | | EpCAM | EVQLVQSGPGLVQ | 460 | DIQMTQSPSSLSAS | 539 |
| | | | PGGSVRISCAASG | | VGDRVTITCRSTKS | |
| | | | YTFTNYGMNWVKQ | | LLHSNGITYLYWYQ | |
| | | | APGKGLEWMGWIN | | QKPGKAPKLLIYQM | |
| | | | TYTGESTYADSFK | | SNLASGVPSRFSSS | |
| | | | GRFTFSLDTSASA | | GSGTDFTLTISSLQ | |
| | | | AYLQINSLRAEDT | | PEDFATYYCAQNLE | |
| | | | AVYYCARFAIKGD | | IPRTFGQGTKVEIK | |
| | | | YWGQGTLLTVSS | | R | |
| MEDI-547 | 1C1 | EphA2 | EVQLLESGGGLVQ | 461 | DIQMTQSPSSLSAS | 540 |
| | | | PGGSLRLSCAASG | | VGDRVTITCRASQS | |
| | | | FTFSHYMMAWVRQ | | ISTWLAWYQQKPGK | |
| | | | APGKGLEWVSRIG | | APKLLIYKASNLHT | |
| | | | PSGGPTHYADSVK | | GVPSRFSGSGSGTE | |
| | | | GRFTISRDNSKNT | | FSLTISGLQPDDFA | |
| | | | LYLQMNSLRAEDT | | TYYCQQYNSYSRTF | |
| | | | AVYYCAGYDSGYD | | GQGTKVEIKR | |
| | | | YVAVAGPAEYFQH | | | |
| | | | WGQGTLVTVSSA | | | |
| MORAb- | farletuzumab | FOLR1 | EVQLVESGGGVVQ | 462 | DIQLTQSPSSLSAS | 541 |
| 003 | | | PGRSLRLSCSASG | | VGDRVTITCSVSSS | |
| | | | FTFSGYGLSWVRQ | | ISSNNLHWYQQKPG | |
| | | | APGKGLEWVAMIS | | KAPKPWIYGTSNLA | |
| | | | SGGSYTYYADSVK | | SGVPSRFSGSGSGT | |
| | | | GRFAISRDNAKNT | | DYTFTISSLQPEDI | |
| | | | LFLQMDSLRPEDT | | ATYYCQQWSSYPYM | |
| | | | GVYFCARHGDDPA | | YTFGQGTKVEIK | |
| | | | WFAYWGQGTPVTV | | | |
| | | | SS | | | |
| M9346A | huMOV19 | FOLR1 | QVQLVQSGAEVVK | 463 | DIVLTQSPLSLAVS | 542 |
| | (vLCv1.00) | | PGASVKISCKASG | | LGQPAIISCKASQS | |
| | | | YTFTGYFMNWVKQ | | VSFAGTSLMHWYHQ | |
| | | | SPGQSLEWIGRIH | | KPGQQPRLLIYRAS | |
| | | | PYDGDTFYNQKFQ | | NLEAGVPDRFSGSG | |
| | | | GKATLTVDKSSNT | | SKTDFTLNISPVEA | |
| | | | AHMELLSLTSEDF | | EDAATYYCQQSREY | |
| | | | AVYYCTRYDGSRA | | PYTFGGGTKLEIKR | |
| | | | MDYWGQGTTVTVS | | | |
| | | | S | | | |
| M9346A | huMOV19 | FOLR1 | QVQLVQSGAEVVK | 464 | DIVLTQSPLSLAVS | 543 |
| | (vLCv1.60) | | PGASVKISCKASG | | LGQPAIISCKASQS | |
| | | | YTFTGYFMNWVKQ | | VSFAGTSLMHWYHQ | |
| | | | SPGQSLEWIGRIH | | KPGQQPRLLIYRAS | |
| | | | PYDGDTFYNQKFQ | | NLEAGVPDRFSGSG | |
| | | | GKATLTVDKSSNT | | SKTDFTLTISPVEA | |
| | | | AHMELLSLTSEDF | | EDAATYYCQQSREY | |
| | | | AVYYCTRYDGSRA | | PYTFGGGTKLEIKR | |
| | | | MDYWGQGTTVTVS | | | |
| | | | S | | | |
| 26B3.F2 | | FOLR1 | GPELVKPGASVKI | 465 | PASLSASVGETVTI | 544 |
| | | | SCKASDYSFTGYF | | TCRTSENIFSYLAW | |
| | | | MNWVMQSHGKSLE | | YQQKQGISPQLLVY | |
| | | | WIGRIFPYNGDTF | | NAKTLAEGVPSRFS | |
| | | | YNQKFKGRATLTV | | GSGSGTQFSLKINS | |
| | | | DKSSSTAHMELRS | | LQPEDFGSYYCQHH | |
| | | | LASEDSAVYFCAR | | YAFPWTFGGGSKLE | |
| | | | GTHYFDYWGQGTT | | IK | |
| | | | LTVSS | | | |
| AMG-595 | | Her1(EGFR) | QVQLVESGGGVVQ | 466 | DTVMTQTPLSSHVT | 545 |
| | | | SGRSLRLSCAASG | | LGQPASISCRSSQS | |
| | | | FTFRNYGMHWVRQ | | LVHSDGNTYLSWLQ | |
| | | | APGKGLEWVAVIW | | QRPGQPPRLLIYRI | |
| | | | YDGSDKYYADSVR | | SRRFSGVPDRFSGS | |
| | | | GRFTISRDNSKNT | | GAGTDFTLEISRVE | |
| | | | LYLQMNSLRAEDT | | AEDVGVYYCMQSTH | |
| | | | AVYYCARDGYDIL | | VPRTFGQGTKVEIK | |
| | | | TGNPRDFDYWGQG | | | |
| | | | TLVTVSS | | | |
| Erubitux ™ | cetutximab | Her1(EGFR) | QVQLKQSGPGLVQ | 467 | DILLTQSPVILSVS | 546 |
| | | | PSQSLSITCTVSG | | PGERVSFSCRASQS | |
| | | | FSLTNYGVHWVRQ | | IGTNIHWYQQRTNG | |
| | | | SPGKGLEWLGVIW | | SPRLLIKYASESIS | |
| | | | SGGNTDYNTPFTS | | GIPSRFSGSGSGTD | |
| | | | RLSINKDNSKSQV | | FTLSINSVESEDIA | |
| | | | FFKMNSLQSNDTA | | DYYCQQNNNWPTTF | |
| | | | IYYCARALTYYDY | | GAGTKLELKR | |
| | | | EFAYWGQGTLVTV | | | |
| | | | SAA | | | |
| GA201 | Imgatuzumab | Her1(EGFR) | QVQLVQSGAEVKK | 468 | DIQMTQSPSSLSAS | 547 |
| | | | PGSSVKVSCKASG | | VGDRVTITCRASQG | |
| | | | FTFTDYKIHWVRQ | | INNYLNWYQQKPGK | |
| | | | APGQGLEWMGYFN | | APKRLIYNTNNLQT | |
| | | | PNSGYSTYAQKFQ | | GVPSRFSGSGSGTE | |
| | | | GRVTITADKSTST | | FTLTISSLQPEDFA | |
| | | | AYMELSSLRSEDT | | TYYCLQHNSFPTFG | |
| | | | AVYYCARLSPGGY | | QGTKLEIKRT | |
| | | | YVMDAWGQGTTVT | | | |
| | | | VSS | | | |
| Humax | zalutumumab | Her1 | QVQLVESGGGVVQ | 469 | AIQLTQSPSSLSAS | 548 |
| | | (EGFR) | PGRSLRLSCAASG | | VGDRVTITCRASQD | |
| | | | FTFSTYGMHWVRQ | | ISSALVWYQQKPGK | |
| | | | APGKGLEWVAVIW | | APKLLIYDASSLES | |
| | | | DDGSYKYYGDSVK | | GVPSRFSGSESGTD | |
| | | | GRFTISRDNSKNT | | FTLTISSLQPEDFA | |
| | | | LYLQMNSLRAEDT | | TYYCQQFNSYPLTF | |
| | | | AVYYCARDGITMV | | GGGTKVEIK | |
| | | | RGVMKDYFDYWGQ | | | |
| | | | GTLVTVSS | | | |
| IMC-11F8 | necitumumab | Her1 | QVQLQESGPGLVK | 470 | EIVMTQSPATLSLS | 549 |
| | | (EGFR) | PSQTLSLTCTVSG | | PGERATLSCRASQS | |
| | | | GSISSGDYYWSWI | | VSSYLAWYQQKPGQ | |
| | | | RQPPGKGLEWIGY | | APRLLIYDASNRAT | |
| | | | IYYSGSTDYNPSL | | GIPARFSGSGSGTD | |
| | | | KSRVTMSVDTSKN | | FTLTISSLEPEDFA | |
| | | | QFSLKVNSVTAAD | | VYYCHQYGSTPLTF | |
| | | | TAVYYCARVSIFG | | GGGTKAEIK | |
| | | | VGTFDYWGQGTLV | | | |
| | | | TVSS | | | |
| MM-151 | P1X | Her1 | QVQLVQSGAEVKK | 471 | DIQMTQSPSTLSAS | 550 |
| | | (EGFR) | PGSSVKVSCKASG | | VGDRVTITCRASQS | |
| | | | GTFSSYAISWVRQ | | ISSWWAWYQQKPGK | |
| | | | APGQGLEWMGSII | | APKLLIYDASSLES | |
| | | | PIFGTVNYAQKFQ | | GVPSRFSGSGSGTE | |
| | | | GRVTITADESTST | | FTLTISSLQPDDFA | |
| | | | AYMELSSLRSEDT | | TYYCQQYHAHPTTF | |
| | | | AVYYCARDPSVNL | | GGGTKVEIK | |
| | | | YWYFDLWGRGTLV | | | |
| | | | TVSS | | | |
| MM-151 | P2X | Her1 | QVQLVQSGAEVKK | 472 | DIVMTQSPDSLAVS | 551 |
| | | (EGFR) | PGSSVKVSCKASG | | LGERATINCKSSQS | |
| | | | GTFGSYAISWVRQ | | VLYSPNNKNYLAWY | |
| | | | APGQGLEWMGSII | | QQKPGQPPKLLIYW | |
| | | | PIFGAANPAQKSQ | | ASTRESGVPDRFSG | |
| | | | GRVTITADESTST | | SGSGTDFTLTISSL | |
| | | | AYMELSSLRSEDT | | QAEDVAVYYCQQYY | |
| | | | AVYYCAKMGRGKV | | GSPITFGGGTKVEI | |
| | | | AFDIWGQGTMVTV | | K | |
| | | | SS | | | |
| MM-151 | P3X | Her1 | QVQLVQSGAEVKK | 473 | EIVMTQSPATLSVS | 552 |
| | | (EGFR) | PGASVKVSCKASG | | PGERATLSCRASQS | |
| | | | YAFTSYGINWVRQ | | VSSNLAWYQQKPGQ | |
| | | | APGQGLEWMGWIS | | APRLLIYGASTRAT | |
| | | | AYNGNTYYAQKLR | | GIPARFSGSGSGTE | |
| | | | GRVTMTTDTSTST | | FTLTISSLQSEDFA | |
| | | | AYMELRSLRSDDT | | VYYCQDYRTWPRRV | |
| | | | AVYYCARDLGGYG | | FGGGTKVEIK | |
| | | | SGSVPFDPWGQGT | | | |
| | | | LVTVSS | | | |
| TheraCIM | nimotuzumab | Her1 | QVQLQQSGAEVKK | 474 | DIQMTQSPSSLSAS | 553 |
| | | (EGFR) | PGSSVKVSCKASG | | VGDRVTITCRSSQN | |
| | | | YTFTNYYIYWVRQ | | IVHSNGNTYLDWYQ | |
| | | | APGQGLEWIGGIN | | QTPGKAPKLLIYKV | |
| | | | PTSGGSNFNEKFK | | SNRFSGVPSRFSGS | |
| | | | TRVTITADESSTT | | GSGTDFTFTISSLQ | |
| | | | AYMELSSLRSEDT | | PEDIATYYCFQYSH | |
| | | | AFYFCTRQGLWFD | | VPWTFGQGTKLQIT | |
| | | | SDGRGFDFWGQGT | | | |
| | | | TVTVSS | | | |
| Vectibix ™ | panitumimab | Her1 | QVQLQESGPGLVK | 475 | DIQMTQSPSSLSAS | 554 |
| | | (EGFR) | PSETLSLTCTVSG | | VGDRVTITCQASQD | |
| | | | GSVSSGDYYWTWI | | ISNYLNWYQQKPGK | |
| | | | RQSPGKGLEWIGH | | APKLLIYDASNLET | |
| | | | IYYSGNTNYNPSL | | GVPSRFSGSGSGTD | |
| | | | KSRLTISIDTSKT | | FTFTISSLQPEDIA | |
| | | | QFSLKLSSVTAAD | | TYFCQHFDHLPLAF | |
| | | | TAIYYCVRDRVTG | | GGGTKVEIKR | |
| | | | AFDIWGQGTMVTV | | | |
| | | | SSA | | | |
| 07D06 | | Her1 | QIQLVQSGPELKK | 476 | DVVMTQTPLSLPVS | 555 |
| | | (EGFR) | PGETVKISCKASG | | LGDQASISCRSSQS | |
| | | | YTFTEYPIHWVKQ | | LVHSNGNTYLHWYL | |
| | | | APGKGFKWMGMIY | | QKPGQSPKLLIYKV | |
| | | | TDIGKPTYAEEFK | | SNRFSGVPDRFSGS | |
| | | | GRFAFSLETSAST | | GSGTDFTLKISRVE | |
| | | | AYLQINNLKNEDT | | AEDLGVYFCSQSTH | |
| | | | ATYFCVRDRYDSL | | VPWTFGGGTKLEIK | |
| | | | FDYWGQGTTLTVS | | | |
| | | | S | | | |
| 12D03 | | Her1 | EMQLVESGGGFVK | 477 | DVVMTQTPLSLPVS | 556 |
| | | (EGFR) | PGGSLKLSCAASG | | LGDQASISCRSSQS | |
| | | | FAFSHYDMSWVRQ | | LVHSNGNTYLHWYL | |
| | | | TPKQRLEWVAYIA | | QKPGQSPKLLIYKV | |
| | | | SGGDITYYADTVK | | SNRFSGVPDRFSGS | |
| | | | GRFTISRDNAQNT | | GSGTDFTLKISRVE | |
| | | | LYLQMSSLKSEDT | | AEDLGVYFCSQSTH | |
| | | | AMFYCSRSSYGNN | | VLTFGSGTKLEIK | |
| | | | GDALDFWGQGTSV | | | |
| | | | TVSS | | | |
| | C1 | Her2 | QVQLVESGGGLVQ | 478 | QSPSFLSAFVGDRI | 557 |
| | | | PGGSLRLSCAASG | | TITCRASPGIRNYL | |
| | | | FTFSSYAMGWVRQ | | AWYQQKPGKAPKLL | |
| | | | APGKGLEWVSSIS | | IYAASTLQSGVPSR | |
| | | | GSSRYIYYADSVK | | FSGSGSGTDFTLTI | |
| | | | GRFTISRDNSKNT | | SSLQPEDFATYYCQ | |
| | | | LYLQMNSLRAEDT | | QYNSYPLSFGGGTK | |
| | | | AVYYCAKMDASGS | | VEIKR | |
| | | | YFNFWGQGTLVTV | | | |
| | | | SS | | | |
| Erbicin | | Her2 | QVQLLQSAAEVKK | 479 | QAVVTQEPSFSVSP | 558 |
| | | | PGESLKISCKGSG | | GGTVTLTCGLSSGS | |
| | | | YSFTSYWIGWVRQ | | VSTSYYPSWYQQTP | |
| | | | MPGKGLEWMGITY | | GQAPRTLIYSTNTR | |
| | | | PGDSDTRYSPSFQ | | SSGVPDRFSGSILG | |
| | | | GQVTISADKSIST | | NKAALTITGAQADD | |
| | | | AYLQWSSLKASDT | | ESDYYCVLYMGSGQ | |
| | | | AVYYCARWRDSPL | | YVFGGGTKLTVLG | |
| | | | WGQGTLVTVSS | | | |
| Herceptin | trastuzumab | Her2 | EVQLVESGGGLVQ | 480 | DIQMTQSPSSLSAS | 559 |
| | | | PGGSLRLSCAASG | | VGDRVTITCRASQD | |
| | | | FNIKDTYIHWVRQ | | VNTAVAWYQQKPGK | |
| | | | APGKGLEWVARIY | | APKLLIYSASFLYS | |
| | | | PTNGYTRYADSVK | | GVPSRFSGSRSGTD | |
| | | | GRFTISADTSKNT | | FTLTISSLQPEDFA | |
| | | | AYLQMNSLRAEDT | | TYYCQQHYTTPPTF | |
| | | | AVYYCSRWGGDGF | | GQGTKVEIKR | |
| | | | YAMDYWGQGTLVT | | | |
| | | | VSSA | | | |
| MAGH22 | margetuximab | Her2 | QVQLQQSGPELVK | 481 | DIVMTQSHKFMSTS | 560 |
| | | | PGASLKLSCTASG | | VGDRVSITCKASQD | |
| | | | FNIKDTYIHWVKQ | | VNTAVAWYQQKPGH | |
| | | | RPEQGLEWIGRIY | | SPKLLIYSASFRYT | |
| | | | PTNGYTRYDPKFQ | | GVPDRFTGSRSGTD | |
| | | | DKATITADTSSNT | | FTFTISSVQAEDLA | |
| | | | AYLQVSRLTSEDT | | VYYCQQHYTTPPTF | |
| | | | AVYYCSRWGGDGF | | GGGTKVEIK | |
| | | | YAMDYWGQGASVT | | | |
| | | | VSS | | | |
| MM-302 | F5 | Her2 | QVQLVESGGGLVQ | 482 | QSVLTQPPSVSGAP | 561 |
| | | | PGGSLRLSCAASG | | GQRVTISCTGSSSN | |
| | | | FTFRSYAMSWVRQ | | IGAGYGVHWYQQLP | |
| | | | APGKGLEWVSAIS | | GTAPKLLIYGNTNR | |
| | | | GRGDNTYYADSVK | | PSGVPDRFSGFKSG | |
| | | | GRFTISRDNSKNT | | TSASLAITGLQAED | |
| | | | LYLQMNSLRAEDT | | EADYYCQFYDSSLS | |
| | | | AVYYCAKMTSNAF | | GWVFGGGTKLTVLG | |
| | | | AFDYWGQGTLVTV | | | |
| | | | SS | | | |
| Perjeta | pertuzumab | Her2 | EVQLVESGGGLVQ | 483 | DIQMTQSPSSLSAS | 562 |
| | | | PGGSLRLSCAASG | | VGDRVTITCKASQD | |
| | | | FTFTDYTMDWVRQ | | VSIGVAWYQQKPGK | |
| | | | APGKGLEWVADVN | | APKLLIYSASYRYT | |
| | | | PNSGGSIYNQRFK | | GVPSRFSGSGSGTD | |
| | | | GRFTLSVDRSKNT | | FTLTISSLQPEDFA | |
| | | | LYLQMNSLRAEDT | | TYYCQQYYIYPYTF | |
| | | | AVYYCARNLGPSF | | GQGTKVEIKRT | |
| | | | YFDYWGQGTLVTV | | | |
| | | | SS | | | |
| MM-121/ | | Her3 | EVQLLESGGGLVQ | 484 | QSALTQPASVSGSP | 563 |
| SAR256212 | | | PGGSLRLSCAASG | | GQSITISCTGTSSD | |
| | | | FTFSHYVMAWVRQ | | VGSYNVVSWYQQHP | |
| | | | APGKGLEWVSSIS | | GKAPKLIIYEVSQR | |
| | | | SSGGWTLYADSVK | | PSGVSNRFSGSKSG | |
| | | | GRFTISRDNSKNT | | NTASLTISGLQTED | |
| | | | LYLQMNSLRAEDT | | EADYYCCSYAGSSI | |
| | | | AVYYCTRGLKMAT | | FVIFGGGTKVTVL | |
| | | | IFDYWGQGTLVTV | | | |
| | | | SS | | | |
| MEHD794 | Duligotumab | Her1 | EVQLVESGGGLVQ | 485 | DIQMTQSPSSLSAS | 564 |
| 5A | | (EGFR)/ | PGGSLRLSCAASG | | VGDRVTITCRASQN | |
| | | Her3 | FTLSGDWIHWVRQ | | IATDVAWYQQKPGK | |
| | | | APGKGLEWVGEIS | | APKLLIYSASFLYS | |
| | | | AAGGYTDYADSVK | | GVPSRFSGSGSGTD | |
| | | | GRFTISADTSKNT | | FTLTISSLQPEDFA | |
| | | | AYLQMNSLRAEDT | | TYYCQQSEPEPYTF | |
| | | | AVYYCARESRVSF | | GQGTKVEIKR | |
| | | | EAAMDYWGQGTLV | | | |
| | | | TVSS | | | |
| MM-111 | | Her2/3 | QVQLQESGGGLVK | 486 | QSALTQPASVSGSP | 565 |
| | | | PGGSLRLSCAASG | | GQSITISCTGTSSD | |
| | | | FTFSSYWMSWVRQ | | VGGYNFVSWYQQHP | |
| | | | APGKGLEWVANIN | | GKAPKLMIYDVSDR | |
| | | | RDGSASYYVDSVK | | PSGVSDRFSGSKSG | |
| | | | GRFTISRDDAKNS | | NTASLIISGLQADD | |
| | | | LYLQMNSLRAEDT | | EADYYCSSYGSSST | |
| | | | AVYYCARDRGVGY | | HVIFGGGTKVTVLG | |
| | | | FDLWGRGTLVTVS | | | |
| | | | S | | | |
| MM-111 | | Her2/3 | QVQLVQSGAEVKK | 487 | QSVLTQPPSVSAAP | 566 |
| | | | PGESLKISCKGSG | | GQ | |
| | | | YSFTSYWIAWVRQ | | KVTISCSGSSSNIG | |
| | | | MPGKGLEYMGLIY | | NNYVSWYQQLPGTA | |
| | | | PGDSDTKYSPSFQ | | PKLLIYDHTNRPAG | |
| | | | GQVTISVDKSVST | | VPDRFSGSKSGTSA | |
| | | | AYLQWSSLKPSDS | | SLATSGFRSEDEAD | |
| | | | AVYFCARHDVGYC | | YYCASWDYTLSGWV | |
| | | | TDRTCAKWPEWLG | | FGGGTKLTVLG | |
| | | | VWGQGTLVTVSS | | | |
| BAY 94- | anetumab | Mesothelin | QVELVQSGAEVKK | 488 | DIALTQPASVSGSP | 567 |
| 9343 | ravtansine | | PGESLKISCKGSG | | GQSITISCTGTSSD | |
| | | | YSFTSYWIGWVRQ | | IGGYNSVSWYQQHP | |
| | | | APGKGLEWMGIID | | GKAPKLMIYGVNNR | |
| | | | PGDSRTRYSPSFQ | | PSGVSNRFSGSKSG | |
| | | | GQVTISADKSIST | | NTASLTISGLQAED | |
| | | | AYLQWSSLKASDT | | EADYYCSSYDIESA | |
| | | | AMYYCARGQLYGG | | TPVFGGGTKLTVL | |
| | | | TYMDGWGQGTLVT | | | |
| | | | VSS | | | |
| MORAb- | amatuximab | Mesothelin | QVQLQQSGPELEK | 489 | DIELTQSPAIMSAS | 568 |
| 009 | | | PGASVKISCKASG | | PGEKVTMTCSASSS | |
| | | | YSFTGYTMNWVKQ | | VSYMHWYQQKSGTS | |
| | | | SHGKSLEWIGLIT | | PKRWIYDTSKLASG | |
| | | | PYNGASSYNQKFR | | VPGRFSGSGSGNSY | |
| | | | GKATLTVDKSSST | | SLTISSVEAEDDAT | |
| | | | AYMDLLSLTSEDS | | YYCQQWSKHPLTFG | |
| | | | AVYFCARGGYDGR | | SGTKVEIKR | |
| | | | GFDYWGSGTPVTV | | | |
| | | | SSA | | | |
| hPAM4- | clivatuzumab | MUC1 | QVQLQQSGAEVKK | 490 | DIQLTQSPSSLSAS | 569 |
| Cide | | | FGASVKVSCEASG | | VGDRVTMTCSASSS | |
| | | | YTFPSYVLHWVKQ | | VSSSYLYWYQQKPG | |
| | | | APGQGLEWIGYIN | | KAPKLWIYSTSNLA | |
| | | | PYNDGTQTNKKFK | | SGVPARFSGSGSGT | |
| | | | GKATLTRDTSINT | | DFTLTISSLQPEDS | |
| | | | AYMELSRLRSDDT | | ASYFCHQWNRYPYT | |
| | | | AVYYCARGFGGSY | | FGGGTRLEIK | |
| | | | GFAYNGQGTLVTV | | | |
| | | | SS | | | |
| SAR566658 | huDS6v1.01 | MUC1 | QAQLQVSGAEVVK | 491 | EIVLTQSPATMSAS | 570 |
| | | | PGASVKMSCKASG | | PGERVTITCSAHSS | |
| | | | YTFTSYNMHWVKQ | | VSFMHWFQQKPGTS | |
| | | | TPGQGLEWIGYIY | | PKLWIYSTSSLASG | |
| | | | PGNGATNYNQKFQ | | VPARFGGSGSGTSY | |
| | | | GKATLTADTSSST | | SLTISSMEAEDAAT | |
| | | | AYMQISSLTSEDS | | YYCQQRSSFPLTFG | |
| | | | AVYFCARGDSVPF | | AGTKLELKR | |
| | | | AYWGQGTLVTVSA | | | |
| Theragyn | Pemtumomab | MUC1 | QVQLQQSGAELMK | 492 | DIVMSQSPSSLAVS | 571 |
| | muHMFG1 | | PGASVKISCKATG | | VGEKVTMSCKSSQS | |
| | | | YTFSAYWIEWVKQ | | LLYSSNQKIYLAWY | |
| | | | RPGHGLEWIGEIL | | QQKPGQSPKLLIYW | |
| | | | PGSNNSRYNEKFK | | ASTRESGVPDRFTG | |
| | | | GKATFTADTSSNT | | GGSGTDFTLTISSV | |
| | | | AYMQLSSLTSEDS | | KAEDLAVYYCQQYY | |
| | | | AVYYCSRSYDFAW | | RYPRTFGGGTKLEI | |
| | | | FAYWGQGTPVTVS | | KR | |
| | | | A | | | |
| Therex | Sontuzumab | MUC1 | QVQLVQSGAEVKK | 493 | DIQMTQSPSSLSAS | 572 |
| | huHMFG1 | | PGASVKVSCKASG | | VGDRVTITCKSSQS | |
| | AS1402 | | YTFSAYWIEWVRQ | | LLYSSNQKIYLAWY | |
| | R1150 | | APGKGLEWVGEIL | | QQKPGKAPKLLIYW | |
| | | | PGSNNSRYNEKFK | | ASTRESGVPSRFSG | |
| | | | GRVTVTRDTSTNT | | SGSGTDFTFTISSL | |
| | | | AYMELSSLRSEDT | | QPEDIATYYCQQYY | |
| | | | AVYYCARSYDFAW | | RYPRTFGQGTKVEI | |
| | | | FAYWGQGTLVTVS | | KR | |
| | | | S | | | |
| MDX-1105 | | PD-L1 | QVQLVQSGAEVKK | 494 | EIVLTQSPATLSLS | 573 |
| or BMS- | | | PGSSVKVSCKTSG | | PGERATLSCRASQS | |
| 936559 | | | DTFSTYAISWVRQ | | VSSYLAWYQQKPGQ | |
| | | | APGQGLEWMGGII | | APRLLIYDASNRAT | |
| | | | PIFGKAHYAQKFQ | | GIPARFSGSGSGTD | |
| | | | GRVTITADESTST | | FTLTISSLEPEDFA | |
| | | | AYMELSSLRSEDT | | VYYCQQRSNWPTFG | |
| | | | AVYFCARKFHFVS | | QGTKVEIK | |
| | | | GSPFGMDVWGQGT | | | |
| | | | TVTVSS | | | |
| MEDI- | durvalumab | PD-L1 | EVQLVESGGGLVQ | 495 | EIVLTQSPGTLSLS | 574 |
| 4736 | | | PGGSLRLSCAASG | | PGERATLSCRASQR | |
| | | | FTFSRYWMSWVRQ | | VSSSYLAWYQQKPG | |
| | | | APGKGLEWVANIK | | QAPRLLIYDASSRA | |
| | | | QDGSEKYYVDSVK | | TGIPDRFSGSGSGT | |
| | | | GRFTISRDNAKNS | | DFTLTISRLEPEDF | |
| | | | LYLQMNSLRAEDT | | AVYYCQQYGSLPWT | |
| | | | AVYYCAREGGWFG | | FGQGTKVEIK | |
| | | | ELAFDYWGQGTLV | | | |
| | | | TVSS | | | |
| MPDL3280A | atezolizumab | PD-L1 | EVQLVESGGGLVQ | 496 | DIQMTQSPSSLSAS | 575 |
| | | | PGGSLRLSCAASG | | VGDRVTITCRASQD | |
| | | | FTFSDSWIHWVRQ | | VSTAVAWYQQKPGK | |
| | | | APGKGLEWVAWIS | | APKLLIYSASFLYS | |
| | | | PYGGSTYYADSVK | | GVPSRFSGSGSGTD | |
| | | | GRFTISADTSKNT | | FTLTISSLQPEDFA | |
| | | | AYLQMNSLRAEDT | | TYYCQQYLYHPATF | |
| | | | AVYYCARRHWPGG | | GQGTKVEIK | |
| | | | FDYWGQGTLVTVS | | | |
| | | | S | | | |
| MSB00107 | avelumab | PD-L1 | EVQLLESGGGLVQ | 497 | QSALTQPASVSGSP | 576 |
| 18C | | | PGGSLRLSCAASG | | GQSITISCTGTSSD | |
| | | | FTFSSYIMMWVRQ | | VGGYNYVSWYQQHP | |
| | | | APGKGLEWVSSTY | | GKAPKLMIYDVSNR | |
| | | | PSGGITFYADTVK | | PSGVSNRFSGSKSG | |
| | | | GRFTISRDNSKNT | | NTASLTISGLQAED | |
| | | | LYLQMNSLRAEDT | | EADYYCSSYTSSST | |
| | | | AVYYCARIKLGTV | | RVFGTGTKVTVL | |
| | | | TTVDYWGQGTLVT | | | |
| | | | VSS | | | |
| MLN591 | | PSMA | EVQLVQSGPEVKK | 498 | DIQMTQSPSSLSTS | 577 |
| | | | PGATVKISCKTSG | | VGDRVTLTCKASQD | |
| | | | YTFTEYTIHWVKQ | | VGTAVDWYQQKPGP | |
| | | | APGKGLEWIGNIN | | SPKLLIYWASTRHT | |
| | | | PNNGGTTYNQKFE | | GIPSRFSGSGSGTD | |
| | | | DKATLTVDKSTDT | | FTLTISSLQPEDFA | |
| | | | AYMELSSLRSEDT | | DYYCQQYNSYPLTF | |
| | | | AVYYCAAGWNFDY | | GPGTKVDIK | |
| | | | WGQGTLLTVSS | | | |
| MT112 | pasotuxizumab | PSMA | QVQLVESGGGLVK | 499 | DIQMTQSPSSLSAS | 578 |
| | | | PGESLRLSCAASG | | VGDRVTITCKASQN | |
| | | | FTFSDYYMYWVRQ | | VDTNVAWYQQKPGQ | |
| | | | APGKGLEWVAIIS | | APKSLIYSASYRYS | |
| | | | DGGYYTYYSDIIK | | DVPSRFSGSASGTD | |
| | | | GRFTISRDNAKNS | | FTLTISSVQSEDFA | |
| | | | LYLQMNSLKAEDT | | TYYCQQYDSYPYTF | |
| | | | AVYYCARGFPLLR | | GGGTKLEIK | |
| | | | HGAMDYWGQGTLV | | | |
| | | | TVSS | | | |
| CC49 | | TAG-72 | QVQLVQSGAEVVK | 500 | DIVMSQSPDSLAVS | 579 |
| (Humanized) | | | PGASVKISCKASG | | LGERVTLNCKSSQS | |
| | | | YTFTDHAIHWVKQ | | LLYSGNQKNYLAWY | |
| | | | NPGQRLEWIGYFS | | QQKPGQSPKLLIYW | |
| | | | PGNDDFKYNERFK | | ASARESGVPDRFSG | |
| | | | GKATLTADTSAST | | SGSGTDFTLTISSV | |
| | | | AYVELSSLRSEDT | | QAEDVAVYYCQQYY | |
| | | | AVYFCTRSLNMAY | | SYPLTFGAGTKLEL | |
| | | | WGQGTLVTVSS | | K | |
| IMC-18F1 | icrucumab | VEGFR1 | QAQVVESGGGVVQ | 501 | EIVLTQSPGTLSLS | 580 |
| | | | SGRSLRLSCAASG | | PGERATLSCRASQS | |
| | | | FAFSSYGMHWVRQ | | VSSSYLAWYQQKPG | |
| | | | APGKGLEWVAVIW | | QAPRLLIYGASSRA | |
| | | | YDGSNKYYADSVR | | TGIPDRFSGSGSGT | |
| | | | GRFTISRDNSENT | | DFTLTISRLEPEDF | |
| | | | LYLQMNSLRAEDT | | AVYYCQQYGSSPLT | |
| | | | AVYYCARDHYGSG | | FGGGTKVEIK | |
| | | | VHHYFYYGLDVWG | | | |
| | | | QGTTVTVSS | | | |
| Cyramza | ramucirumab | VEGFR2 | EVQLVQSGGGLVK | 502 | DIQMTQSPSSVSAS | 581 |
| | | | PGGSLRLSCAASG | | IGDRVTITCRASQG | |
| | | | FTFSSYSMNWVRQ | | IDNWLGWYQQKPGK | |
| | | | APGKGLEWVSSIS | | APKLLIYDASNLDT | |
| | | | SSSSYIYYADSVK | | GVPSRFSGSGSGTY | |
| | | | GRFTISRDNAKNS | | FTLTISSLQAEDFA | |
| | | | LYLQMNSLRAEDT | | VYFCQQAKAFPPTF | |
| | | | AVYYCARVTDAFD | | GGGTKVDIKR | |
| | | | IWGQGTMVTVSSA | | | |
| g165 DFM- | alacizumab | VEGFR2 | EVQLVESGGGLVQ | 503 | DIQMTQSPSSLSAS | 582 |
| PEG | pegol | | PGGSLRLSCAASG | | VGDRVTITCRASQD | |
| | | | FTFSSYGMSWVRQ | | IAGSLNWLQQKPGK | |
| | | | APGKGLEWVATIT | | AIKRLIYATSSLDS | |
| | | | SGGSYTYYVDSVK | | GVPKRFSGSRSGSD | |
| | | | GRFTISRDNAKNT | | YTLTISSLQPEDFA | |
| | | | LYLQMNSLRAEDT | | TYYCLQYGSFPPTF | |
| | | | AVYYCVRIGEDAL | | GQGTKVEIK | |
| | | | DYWGQGTLVTVSS | | | |
| Imclone | | VEGFR2 | KVQLQQSGTELVK | 504 | DIVLTQSPASLAVS | 583 |
| 6.64 | | | PGASVKVSCKASG | | LGQRATISCRASES | |
| | | | YIFTEYIIHWVKQ | | VDSYGNSFMHWYQQ | |
| | | | RSGQGLEWIGWLY | | KPGQPPKLLIYRAS | |
| | | | PESNIIKYNEKFK | | NLESGIPARFSGSG | |
| | | | DKATLTADKSSST | | SRTDFTLTINPVEA | |
| | | | VYMELSRLTSEDS | | DDVATYYCQQSNED | |
| | | | AVYFCTRHDGTNF | | PLTFGAGTKLELKR | |
| | | | DYWGQGTTLTVSS | | | |
| | | | A | | | |
| huOKT3 | | CD3 | QVQLVQSGGGVVQ | 505 | DIQMTQSPSSLSAS | 584 |
| | | | PGRSLRLSCKASG | | VGDRVTITCSASSS | |
| | | | YTFTRYTMHWVRQ | | VSYMNWYQQTPGKA | |
| | | | APGKGLEWIGYIN | | PKRWIYDTSKLASG | |
| | | | PSRGYTNYNQKVK | | VPSRFSGSGSGTDY | |
| | | | DRFTISRDNSKNT | | TFTISSLQPEDIAT | |
| | | | AFLQMDSLRPEDT | | YYCQQWSSNPFTFG | |
| | | | GVYFCARYYDDHY | | QGTKLQITR | |
| | | | CLDYWGQGTPVTV | | | |
| | | | SS | | | |
| huUCHT1 | | CD3 | EVQLVESGGGLVQ | 506 | DIQMTQSPSSLSAS | 585 |
| | | | PGGSLRLSCAASG | | VGDRVTITCRASQD | |
| | | | YSFTGYTMNWVRQ | | IRNYLNWYQQKPGK | |
| | | | APGKGLEWVALIN | | APKLLIYYTSRLES | |
| | | | PYKGVSTYNQKFK | | GVPSRFSGSGSGTD | |
| | | | DRFTISVDKSKNT | | YTLTISSLQPEDFA | |
| | | | AYLQMNSLRAEDT | | TYYCQQGNTLPWTF | |
| | | | AVYYCARSGYYGD | | GQGTKVEIK | |
| | | | SDWYFDVWGQGTL | | | |
| | | | VTVSS | | | |
| hu12F6 | | CD3 | QVQLVQSGGGVVQ | 507 | DIQMTQSPSSLSAS | 586 |
| | | | PGRSLRLSCKASG | | VGDRVTMTCRASSS | |
| | | | YTFTSYTMHWVRQ | | VSYMHWYQQTPGKA | |
| | | | APGKGLEWIGYIN | | PKPWIYATSNLASG | |
| | | | PSSGYTKYNQKFK | | VPSRFSGSGSGTDY | |
| | | | DRFTISADKSKST | | TLTISSLQPEDIAT | |
| | | | AFLQMDSLRPEDT | | YYCQQWSSNPPTFG | |
| | | | GVYFCARWQDYDV | | QGTKLQITR | |
| | | | YFDYWGQGTPVTV | | | |
| | | | SS | | | |
| mOKT3 | | CD3 | QVQLQQSGAELAR | 508 | QIVLTQSPAIMSAS | 587 |
| | | | PGASVKMSCKASG | | PGEKVTMTCSASSS | |
| | | | YTFTRYTMHWVKQ | | VSYMNWYQQKSGTS | |
| | | | RPGQGLEWIGYIN | | PKRWIYDTSKLASG | |
| | | | PSRGYTNYNQKFK | | VPAHFRGSGSGTSY | |
| | | | DKATLTTDKSSST | | SLTISGMEAEDAAT | |
| | | | AYMQLSSLTSEDS | | YYCQQWSSNPFTFG | |
| | | | AVYYCARYYDDHY | | SGTKLEINR | |
| | | | CLDYWGQGTTLTV | | | |
| | | | SS | | | |
| MT103 | blinatumomab | CD3 | DIKLQQSGAELAR | 509 | DIQLTQSPAIMSAS | 588 |
| | | | PGASVKMSCKTSG | | PGEKVTMTCRASSS | |
| | | | YTFTRYTMHWVKQ | | VSYMNWYQQKSGTS | |
| | | | RPGQGLEWIGYIN | | PKRWIYDTSKVASG | |
| | | | PSRGYTNYNQKFK | | VPYRFSGSGSGTSY | |
| | | | DKATLTTDKSSST | | SLTISSMEAEDAAT | |
| | | | AYMQLSSLTSEDS | | YYCQQWSSNPLTFG | |
| | | | AVYYCARYYDDHY | | AGTKLELK | |
| | | | CLDYWGQGTTLTV | | | |
| | | | SS | | | |
| MT110 | solitomab | CD3 | DVQLVQSGAEVKK | 510 | DIVLTQSPATLSLS | 589 |
| | | | PGASVKVSCKASG | | PGERATLSCRASQS | |
| | | | YTFTRYTMHWVRQ | | VSYMNWYQQKPGKA | |
| | | | APGQGLEWIGYIN | | PKRWIYDTSKVASG | |
| | | | PSRGYTNYADSVK | | VPARFSGSGSGTDY | |
| | | | GRFTITTDKSTST | | SLTINSLEAEDAAT | |
| | | | AYMELSSLRSEDT | | YYCQQWSSNPLTEG | |
| | | | ATYYCARYYDDHY | | GGTKVEIK | |
| | | | CLDYWGQGTTVTV | | | |
| | | | SS | | | |

*underlined sequences, if present, are CDRs within the VL and VH

TABLE 20
Intramolecular Linkers

| Linker | Name | Amino Acid Sequence | SEQ ID NO: |
|---|---|---|---|
| L-1 | Y30 | GSGEGSEGEGGGEGSEGEGSGEGGEGEGSG | 590 |
| L-2 | GS20 | GGGGSGGGGSGGGGSGGGGS | 591 |
| L-3 | GS15 | GGGGSGGGGSGGGGS | 592 |
| L-4 | AE30_1 | AGSPTSTEEGTSESATPESGPGSEPATSGS | 593 |
| L-5 | AE30_2 | GTSTEPSEGSAPGTSESATPESGPGSEPAT | 594 |
| L-6 | AE30_3 | GSETPGTSESATPESGPGTSTEPSEGSAPG | 595 |
| L-7 | AG30_1 | GTGPGTPGSGTASSSPGSSTPSGATGSPGP | 596 |
| L-8 | AG30_2 | GSGTASSSPGSSTPSGATGSPGSSPSASTG | 597 |
| L-9 | AG30_3 | GASPGTSSTGSPGTPGSGTASSSPGSSTPS | 598 |
| L-10 | AG30_4 | GTSSTGSPGTPGSGTASSSPGSSTPSGATG | 599 |

(i) Exemplary Targeting Moieties

The following section provides a non-limiting list and description of exemplary targeting moieties and their use in targeted conjugate compositions.

Anti-Her2:

In one embodiment, the invention provides an isolated anti-Her2 targeting moiety. “Anti-Her2” means a targeting moiety that specifically binds to the extracellular domain of the HER2/neu receptor (a.k.a. erbB-2 protein), including, but not limited to antibodies, antibody fragments, fragment dimers, traps, and other polypeptides with binding affinity to the extracellular domain of the HER2/erbB-2 protein. In a preferred embodiment, the anti-Her2 targeting moiety is a scFv. The HER2-encoding gene is found on band q21 of chromosome 17, generates a messenger RNA (MRNA) of 4.8 kb, and the protein encoded by the HER2 gene is 185,000 Daltons. In normal subject, ligands that bind to the HER2 receptor promote dimerization with other receptors, resulting in signal transduction and activation of the PI3K/Akt pathway and the MAPK pathway.

In approximately 25% of breast cancers, the HER2 gene is amplified by 2-fold to greater than 20-fold in each tumor cell nucleus relative to the number of copies of chromosome 17. Amplification of the HER2gene drives protein expression and the resulting increase in the number of receptors at the tumor-cell surface promotes receptor activation, leading to signaling, excessive cellular division, and the formation of tumors (Hicks, D G et al., HER2+ breast cancer: review of biologic relevance and optimal use of diagnostic tools. Am J Clin Pathol. (2008) 129(2):263-73).

The anti-Her2 targeting moiety used as a fusion partner with XTEN creates a composition that has therapeutic utility when administered to a subject by binding to the extracellular domain of the extracellular segment of the HER2/neu receptor and delivering a bioactive payload to the target tissue. In addition, such binding can interfere with receptor dimerization and the resulting activation of EGFR intrinsic tyrosine kinase function (Yarden et al, Biochemistry, (1988), 27, 3114-3118; Schlessinger, Biochemistry, (1988), 27, 3119-3123), with the result that cells with bound receptors undergo arrest during the G1 phase of the cell cycle so there is reduced proliferation of tumor cells, as well as suppression of angiogenesis.

One object of the invention is to provide novel anti-Her2 targeting moieties comprising one or more binding moieties that specifically bind to erbB-2 protein expressed on tumor cells and that do not substantially bind to normal human cells, which may be utilized for the treatment or prevention of erbB-2 expressing cancers, or for the detection of erbB-2 expressing tumor cells. The variable domain CDR and FR residues of a humanized HER2 antibody have been reported in Carter et al., Proc. Natl. Acad. Sci. USA, 89:4285 (1992). In one embodiment, the anti-Her2 TM compositions comprise a single anti-Her2 targeting moiety linked to the conjugate composition. In another embodiment, the anti-Her2 compositions comprise a first and a second anti-Her2 targeting moiety, which may be the same or which may bind different epitopes of the erbB-2 protein. In one embodiment, the anti-Her2 targeting moiety component of a conjugate composition comprises one or more complementarity determining regions (CDRs) of trastuzumab capable of binding to the domain IV of the extracellular segment of the HER2/neu receptor linked to the conjugate composition.

Another embodiment of the invention relates to a method of inhibiting growth of tumor cells by administering to a patient a therapeutically effective amount of anti-Her2-targeted conjugate composition capable of inhibiting the HER2 receptor function and delivering a cytotoxic payload to the tumor cells, thereby effecting death of the cells. In another embodiment, the invention provides a method for the treatment and/or prevention of erbB-2 receptor over-expressing tumors comprising the administration of therapeutically-effective amounts of anti-Her2 conjugate composition comprising a first and a second anti-Her2 binding moiety, which may be identical or which may be distinct and bind different epitopes of the erbB-2 protein, capable of inhibiting the HER2 receptor function. Preferably, such combinations of TM will result in more selective delivery of the associated payload agent to the target tumor and exhibit better cytotoxic activity than would be expected for the sum of the cytotoxic activity of the conjugates with individual TMs at the same overall concentration. Additionally, one or more of the administered conjugate compositions may be conjugated to a radionuclide.

Anti-cMet:

In another embodiment, the invention provides an isolated anti-cMet targeting moiety. “Anti-cMet” means a targeting moiety that specifically binds to Met, or hepatocyte growth factor (HGF) receptor. MET is a proto-oncogene, with the encoded hepatocyte growth factor receptor (HGFR) or cMet having tyrosine-kinase activity essential for embryonic development and wound healing. Upon HGF binding and stimulation, MET induces several biological responses that collectively give rise to invasive growth. Abnormal MET activation in cancer correlates with poor prognosis, where aberrantly active MET triggers tumor growth, angiogenesis and formation of new blood vessels that supply the tumor with nutrients, and cancer spread to other organs (metastasis). MET is deregulated in many types of human malignancies, including cancers of kidney, liver, stomach, breast, and brain. Anti-cMET can be an targeting moiety that specifically binds to a HGF receptor, serving as an antagonist to HGF. In a preferred embodiment, the anti-cMET targeting moiety is a scFv. The anti-cMET can be used as a fusion partner to create a fusion protein conjugate composition that has prophylactic or therapeutic utility when administered to a subject for the treatment of MET-expressing tumors. In one embodiment, the anti-cMET component of an conjugate composition comprises one or more complementarity determining regions (CDRs) of the antibody MetMab or PRO143966. Antibodies to cMet and their sequences have been described in U.S. Pat. No. 5,686,292. U.S. Pat. No. 6,468,529 U.S. Pat. No. 7,476,724 and U.S. Patent Application Publication No. 20070092520.

Anti-IL6R:

In another embodiment, the invention provides an isolated anti-IL6R targeting moiety. “Anti-IL6R” means a targeting moiety that specifically binds to an IL-6 receptor. In a preferred embodiment, the anti-IL6R targeting moiety is a scFv. Anti-IL6R can serve as an antagonist to IL-6. The anti-IL6R can be used as a fusion partner to create a conjugate composition that has prophylactic or therapeutic utility when administered to a subject for inflammatory conditions, such as arthritis or Crohn's disease. Tocilizuma has been shown to have clinical utility in moderate to severe rheumatoid arthritis, and has been approved by the FDA. In one embodiment, the anti-IL6R component of a conjugate composition comprises one or more complementarity determining regions (CDRs) of tocilizuma. Antibodies to IL-6R have been described in U.S Pat. Nos. 5,670,373, 5,795,965, 5,817,790, and 7,479,543.

Anti-IL17:

In another embodiment, the invention provides an isolated anti-IL17 targeting moiety. “Anti-IL17” means a targeting moiety that specifically binds to the cytokine IL-17. In a preferred embodiment, the anti-IL17 targeting moiety is a scFv. IL-17 is a disulfide-linked homodimeric cytokine of about 32 kDa which is synthesized and secreted only by CD4+activated memory T cells (reviewed in Fossiez et al., Int. Rev. Immunol., 16: 541-551 (1998)). Interleukin (IL-17) is a pro-inflammatory T cell cytokine that is expressed, for example, in the synovial fluid of patients with rheumatoid arthritis. IL-17 is a potent inducer of various cytokines such as TNF and IL-1, and IL-17 has been shown to have additive or even synergistic effects with TNF and IL-1. The anti-IL17 can be used as a fusion partner to create a conjugate composition that has prophylactic or therapeutic utility when administered to a subject for inflammatory conditions, such as arthritis or Crohn's disease, or in multiple sclerosis. LY2439821 is an antibody that has shown utility, when added to oral DMARDs, in improving signs and symptoms of rheumatoid arthritis. In one embodiment, the anti-IL6R component of a targeting moiety comprises one or more complementarity determining regions (CDRs) of LY2439821. Anti-IL17 antibodies have been described in US Patent Application Nos. 20050147609 and 20080269467 and PCT application publication WO 2007/070750.

IL17R:

In another embodiment, the invention provides an isolated IL17R targeting moiety. “IL17R” means a targeting moiety that specifically binds to the cytokine receptor for IL-17. In a preferred embodiment, the anti-IL17R targeting moiety is a scFv. Studies have shown that contacting T cells with a soluble form of the IL-17 receptor polypeptide inhibited T cell proliferation and IL-2 production induced by PHA, concanavalin A and anti-TCR monoclonal antibody (Yao et al., J. Immunol., 155:5483-5486 [1995]). As interleukin (IL-17) is a pro-inflammatory T cell cytokine that is a potent inducer of various cytokines such as TNF and IL-1, the IL17R can be used as a fusion partner to create aconjugate composition to bind and neutralize IL-17. The IL17R can have therapeutic utility when administered to a subject for inflammatory conditions, such as rheumatoid arthritis or Crohn's disease. IL7R receptors and homologs have been cloned, as described in U.S. Pat. No. 5,869,286.

Anti-IL12:

In another embodiment, the invention provides an isolated anti-IL12 targeting moiety. “Anti-IL12” means a targeting moiety that specifically binds to the cytokine IL-12 and, in some cases, IL-23. In a preferred embodiment, the anti-IL12 targeting moiety is a scFv. Biologically active IL-12 exists as a heterodimer comprised of 2 covalently linked subunits of 35 (p35) and 40 (p40) kD, the latter being known as IL-23. IL-12 is a cytokine that is an important part of the inflammatory response, and stimulates the production of interferon-gamma (IFN-γ) and tumor necrosis factor-alpha (TNF-a) from T and natural killer (NK) cells, and reduces IL-4 mediated suppression of IFN-γ. T cells that produce IL-12 have a coreceptor, CD30, which is associated with IL-12 activity. IL-12 has also been linked with autoimmunity and with psoriasis, with the interaction between T lymphocytes and stem cell keratinocytes that produce IL-12 being of significance. Ustekinumab is an anti-IL12/23 antibody that has demonstrated utility in the treatment of moderate to severe plaque psoriasis, and has been approved by the FDA. The anti-IL-12 can be used as a fusion partner with XTEN to create a fusion protein composition that has therapeutic utility when administered to a subject suffering from inflammatory conditions, such as, but not limited to, psoriasis, rheumatoid arthritis or Crohn's disease. In one embodiment, the anti-IL12 component of a conjugate composition comprises one or more complementarity determining regions (CDRs) of the antibody ustekinumab. Antibodies to IL-12 and their use have been described in U.S. Pat. No. 7,279,157.

Anti-IL23:

In another embodiment, the invention provides an isolated anti-IL23 targeting moiety. “Anti-IL23” means a targeting moiety that specifically binds to the cytokine IL-23. In a preferred embodiment, the anti-IL23 targeting moiety is a scFv. IL-23 is the name given to a factor that is composed of the p40 subunit of IL-12, and is a pro-inflammatory cytokine that is an important part of the inflammatory response against infection. IL-23 promotes upregulation of the matrix metalloprotease MMP9, increases angiogenesis and reduces CD8+ T-cell infiltration. IL-23 has been demonstrated to play a role in psoriasis, multiple sclerosis and inflammatory bowel. Ustekinumab is an anti-IL23 antibody that has demonstrated utility in psoriasis. The anti-IL-23 can be used as a fusion partner with XTEN to create a fusion protein composition that has therapeutic utility when administered to a subject suffering from inflammatory conditions, such as, but not limited to, psoriasis, rheumatoid arthritis or Crohn's disease. In one embodiment, the anti-IL23 component of a conjugate composition comprises one or more complementarity determining regions (CDRs) of the antibody ustekinumab. Antibodies to IL-23 have been described in U.S. Pat. Nos. 7,491,391 and 7,247,711.

CTLA4:

In another embodiment, the invention provides an isolated CTLA4 targeting moiety. “CTLA4” means a targeting moiety that specifically binds to CD80 and CD86 on antigen-presenting cells, and can specifically bind B7. In a preferred embodiment, the anti-CTLA4 targeting moiety is a scFv. The CTLA4 targeting moiety can be used as a fusion partner to create a conjugate composition that has therapeutic utility when administered to a subject suffering from inflammatory conditions, such as, but not limited to, rheumatoid arthritis, psoriasis and in organ transplantation. Belatacept is a fusion protein composed of the Fc fragment of a human IgG1 immunoglobulin linked to the extracellular domain of CTLA-4 that has shown efficacy in providing extended graft survival. In one embodiment, the CD80 and/or CD86 binding component of a conjugate composition comprises one or more binding domains from belatacept. The cloning and use of CTLA4 compositions have been described in U.S. Pat. Nos. 5,434,131, 5,773,253, 5,851,795, 5,885,579, 7,094,874, and 7,439,230.

Anti-CD3:

In another embodiment, the invention provides an isolated anti-CD3 targeting moiety. “Anti-CD3” means a targeting moiety that specifically binds to CD3 T-cell receptor. In a preferred embodiment, the anti-CD3 targeting moiety is a scFv. T-Cell Co-Receptor is a protein complex composed of four distinct chains; a CD3γ chain, a CD3δ chain, and two CD3ε chains. These chains associate with a molecule known as the T cell receptor (TCR) and the -chain to generate an activation signal in T lymphocytes. Anti-CD3 monoclonal antibodies suppress immune responses by transient T-cell depletion and antigenic modulation of the CD3/T-cell receptor complex. For example, anti-CD3 treatment of adult nonobese diabetic (NOD) mice, a spontaneous model of T-cell-mediated autoimmune insulin-dependent diabetes mellitus, can inhibit the autoimmune process leading to diabetes. The use of anti-CD3 antibodies to treat diseases and disorders has been described, for example, in U.S. Pat. No. 4,515,893. In one embodiment, the CD3 binding component of a conjugate composition comprises one or more complementarity determining regions (CDRs) of the antibody Muromonab-CD3.

Anti-CD40:

In another embodiment, the invention provides an isolated anti-CD40 targeting moiety. “Anti-CD40” means a targeting moiety that specifically binds to the cell-surface receptor CD-40. In a preferred embodiment, the anti-CD40 targeting moiety is a scFv. CD-40 is a cell-surface receptor that plays a role in immune responses, as well as cell growth and survival signaling when activated by CD40 ligand (CD4OL). CD40 is commonly over-expressed and activated in B-cell malignancies, such as multiple myeloma and lymphoma. The anti-CD40 can be used as a fusion partner to create a conjugate composition that can have therapeutic utility when administered to a subject suffering from various cancers, particularly B-cell malignancies. In one embodiment, the anti-CD40 component of a conjugate composition comprises one or more complementarity determining regions (CDRs) of the antibody lucatumumab. Anti-CD40 antibodies have been described in U.S. Pat. No. 7,445,780, and U.S. Patent Appl. Nos. 20070110754 and 20080254026.

Anti-TNFalpha:

In another embodiment, the invention provides an isolated anti-TNFalpha targeting moiety. “Anti-TNFalpha” means a targeting moiety that specifically binds to the cytokine TNFalpha. In a preferred embodiment, the anti-TNFalpha targeting moiety is a scFv. TNFalpha, or cachexin, is a pro-inflammatory cytokine involved in systemic inflammation and is a member of a group of cytokines that stimulate the acute phase reaction. The primary role of TNF is in the regulation of immune cells. TNF is produced mainly by macrophages, but is also produced by lymphoid cells, mast cells, endothelial cells, cardiac myocytes, adipose tissue, fibroblasts, and neuronal tissue. Large amounts of TNF are released in response to lipopolysaccharide and Interleukin-1 (IL-1). TNF has been implicated in autoimmune disorders such as rheumatoid arthritis, ankylosing spondylitis, Crohn's disease, psoriasis and refractory asthma, and plays a role in septic shock and other serious forms of acute inflammatory response and SIRS. The anti-IL-TNFalpha can be used as a fusion partner to create a conjugate composition that can have therapeutic utility in a wide variety of inflammatory disorders, including rheumatoid arthritis, ankylosing spondylitis, Crohn's disease, psoriasis and refractory asthma. Anti-TNFalpha antibodies, such as infliximab and etanercept have shown efficacy in psoriasis, Crohn's disease, ankylosing spondylitis, psoriatic arthritis, rheumatoid arthritis and ulcerative colitis. In one embodiment, the anti-TNFalpha component of a conjugate composition comprises one or more complementarity determining regions (CDRs) or binding regions of the infliximab or etanercept. Anti-TNF antibodies have been described in U.S. Pat. No. 6,790,444, and chimeric antibodies comprising a TNF receptor have been described in U.S. Pat. No. 5,605,690.

The invention provides targeting moiety compositions in which the binding regions of the foregoing referenced exemplary targeting moieties are sequence variants. For example, it will be appreciated that various amino acid deletions, insertions and substitutions can be made in the targeting moiety to create variants without departing from the spirit of the invention with respect to the binding activity or the pharmacologic properties of the targeting moiety. Examples of conservative substitutions for amino acids in polypeptide sequences are shown in Table 21. However, in embodiments of the targeting moiety in which the sequence identity of the targeting moiety is less than 100% compared to a specific sequence referenced or disclosed herein, the invention contemplates substitution of any of the other 19 natural L-amino acids for a given amino acid residue of the given targeting moiety, which may be at any position within the sequence of the targeting moiety or binding region of the targeting moiety, including adjacent amino acid residues. If any one substitution results in an undesirable change in binding activity, then one of the alternative amino acids can be employed and the construct protein evaluated by the methods described herein (e.g., the assays of the Examples), or using any of the techniques and guidelines for conservative and non-conservative mutations set forth, for instance, in U.S. Pat. No. 5,364,934, the contents of which is incorporated by reference in its entirety, or using methods generally known in the art. In addition, variants can include, for instance, polypeptides wherein one or more amino acid residues are added or deleted at the N- or C-terminus of the referenced or disclosed amino acid sequence of a targeting moiety that retains some if not all of the binding activity of the referenced or disclosed targeting moiety; e.g., the ability to bind a target of Tables 2, 3, 4,18, or 19.

TABLE 21
Exemplary conservative amino acid substitutions

| Original Residue | Exemplary Substitutions |
|---|---|
| Ala (A) | val; leu; ile |
| Arg (R) | Lys; Gln; Asn |
| Asn (N) | Gln; His; Llys; Arg |
| Asp (D) | Glu |
| Cys (C) | Ser |
| Gln (Q) | Asn |
| Glu (E) | Asp |
| Gly (G) | Pro |
| His (H) | Asn; Gln; Lys; Arg |
| Ile (I) | Leu; Val; Met; Ala; Phe; Norleucine |
| Leu (L) | Norleucine; Ile: Val; Met; Ala: Phe |
| Lys (K) | Arg′ Gln; Asn |
| Met (M) | Leu; Phe; Ile |
| Phe (F) | Leu; Val; i = Lle; Ala |
| Pro (P) | Gly |
| Ser (S) | Thr |
| Thr (T) | Ser |
| Trp (W) | Tyr |
| Tyr(Y) | Trp; Phe: Thr; Ser |
| Val (V) | Ile; Leu; Met; Phe; Ala; Norleucine |

(ii) Exemplary Forms of Targeting Moieties

The following section provides a non-limiting list and description of exemplary forms of targeting moieties.

“Antibody” or “antibodies”, as used here, refers to a targeting moiety consisting of one or more polypeptides substantially encoded by immunoglobulin genes or fragments of immunoglobulin genes, and is used in the broadest sense to cover intact monoclonal antibodies, multispecific antibodies (e.g. bispecific antibodies) formed from at least two intact antibodies or fragment thereof, and antibody fragments, scFv, diabodies and other forms of synthetic TM so long as they exhibit the desired biological activity; e.g., binding affinity to a target ligand or antigen.

Immunoglobulins can be assigned to different classes depending on the amino acid sequence of the constant domain of their heavy chains. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant domains that correspond to the different classes of immunoglobulins are called a, 6, c, y, and μ, respectively. The subunit structures and three-dimensional configurations of different classes of immunoglobulins are well known.

The term “monoclonal” indicates the character of the targeting moiety antibody or antibody fragment as being obtained from a substantially homogeneous population of antibodies or fragments, and is not to be interpreted as requiring production of the antibody by a particular method. For example, while the monoclonal antibodies created in accordance with the methods of the present invention may be made by the hybridoma method first described by Kohler et al., Nature, 256:495 (1975), they may also be synthetics made by recombinant DNA methods (see, e.g., U.S. Pat. No. 4,816,567) and expressed in either mammalian or non-mammalian hosts; e.g., E. coli. The substitution of immortalized cells with bacterial cells considerably simplifies procedures for preparing large amounts of the inventive binding fusion protein molecules. Furthermore, a recombinant production system allows the ability to produce tailor-made antibodies and fragments thereof, or even libraries to screen for specific attributes. For example, it is possible to produce chimeric molecules with new combinations of binding and effector functions, humanized antibodies and novel antigen-binding molecules, including bifunctional binding fusion proteins. Furthermore, the use of polymerase chain reaction (PCR) amplification (Saiki, R. K., et al., Science 239, 487-491 (1988)) to introduce variations into the sequence and isolate antibody producing sequences from cells has great potential for speeding up the timescale under which specificities can be isolated. Amplified V_{H} and V_{L} genes can be cloned directly into vectors for expression in bacteria or mammalian cells (Orlandi, R., et al., 1989, Proc. Natl. Acad. Sci., USA 86, 3833-3837; Ward, E. S., et al., 1989 supra; Larrick, J. W., et al., 1989, Biochem. Biophys. Res. Commun 160, 1250-1255; Sastry, L. et al., 1989, Proc. Natl. Acad. Sci., USA, 86, 5728-5732). Soluble antibody fragments secreted from bacteria can then be screened in binding assays described herein, or others known in the art, to select those constructs with binding activities sufficient to meet the application.

The monoclonal antibodies herein specifically include “chimeric” antibodies in which a portion of the heavy and/or light chain is identical with or has a high degree of homology to corresponding parental sequences in antibodies derived from a particular first species, while the remainder of the chain(s) is identical with or has a high degree of homology to sequences in antibodies derived from a second species, wherein the resulting antibody exhibits the desired biological activity; e.g., binding affinity for the target antigen or ligand (U.S. Pat. No. 4,816,567; Morrison et al., Proc. Natl. Acad. Sci. USA, 81:6851-4855 (1984)).

The term “humanized” means forms of antibodies, including fragments, that are chimeric in that they include minimal sequence derived from non-human immunoglobulin but otherwise comprise sequence from human immunoglobulins. Humanization is a method to reduce adverse immune reactions to non-human immunoglobulin drugs and other biologics containing non-human amino acid sequences. Methods for humanizing non-human antibodies have been described in the art. Preferably, a humanized antibody contains one or more amino acid residues from a source which is non-human (e.g., murine, rat, or non-human primate) and that are typically taken from a variable domain of a V_{L} or V_{H} chain having the desired specificity and affinity for the target ligand. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting non-human hypervariable region sequences for the corresponding sequences of a human antibody (grafting). Accordingly, such “humanized” antibodies are chimeric antibodies (see, e.g., U.S. Pat. No. 4,816,567) wherein all or a portion of the human variable domain has been substituted by the corresponding sequence from a non-human species. In practice, humanized antibodies are typically human antibodies in which some hypervariable CDR residues and possibly some FR residues are substituted by residues from analogous sites in rodent (or other non-human species, e.g., non-human primates) antibodies. In one embodiment, humanized antibodies comprise residues that are not found in the recipient antibody or in the donor antibody to, for example, increase binding affinity or some other property. In general, humanized antibodies comprise substantially all of at least one, and typically two, variable domains, in which all or substantially all of the hypervariable loops (CDRs) correspond to or have sequences derived from those of a non-human immunoglobulin and all or substantially all of the FR regions are those of a human immunoglobulin sequence. In the case of an svFv made from the humaninzed antibody, the variable light and variable heavy chains are typically linked with a linker, which can be a linker of Table 20 or a fragment of an XTEN from Table 10. The humanized antibody can optionally comprise at least a portion of an immunoglobulin constant region (Fc), preferably that of a human immunoglobulin.

The targeting moieties of the subject compositions can be derived from humanized antibodies. The choice of human variable domains, both light and heavy, to be used in the compositions is very important to reduce immunogenicity of the antibody. For example, the sequence of the variable domain of a rodent antibody can be aligned to a set of known human variable-domain sequences in order to select a human variable domain sequence that is both less likely to elicit an immune response in the recipient and most likely to accept the grafted rodent sequences to form a functional antibody that has inherited the physiochemical properties of the parental rodent antibody. In a corresponding fashion, the human sequence that is closest to that of the rodent can be used as the human framework (FR) for the humanized antibody (Sims et al., J. Immunol, 151:2296 (1993); Chothia et J. Mol. Biol ., 196:901 (1987)). The same framework may be used for several different humanized antibodies (Carter et al., Proc. Natl. Acad. Sci. USA, 89:4285 (1992); Presta et al., J. Immnol., 151:2623 (1993)).

An additional property is that targeting moieties can be humanized yet retain high affinity for the antigen and other favorable biological properties. To achieve this goal, according to a preferred method, humanized targeting moieties are prepared by an iterative process of analysis of the parental sequences and various conceptual humanized products using three-dimensional models of the parental and humanized sequences followed by testing. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and donor using standard recombinant DNA techniques so that the desired characteristic, such as increased affinity for the target antigen(s), can be achieved. In one embodiment, targeting moiety constructs are created in which a sequence comprising linked heavy chain variable domains is linked to a heavy chain constant domain and a sequence comprising linked light chain variable domains is linked to a light chain constant domain (referred to in this embodiment as a fusion protein). Preferably the constant domains are human heavy chain constant domain and human light chain constant domain respectively. In a further embodiment of the foregoing, the targeting moiety can be designed to include portions or all of an immunoglobulin hinge region in order to permit dimerization of the binding fusion protein, which then can be linked to the N-terminus of the CCD region. In an alternative embodiment, the binding fusion protein can be designed to incorporate a partial Fc without a hinge and with a CH2 domain that is truncated but retains FcRn binding in order to confer longer terminal half-life on the construct. In yet another embodiment, the binding fusion protein can be designed to incorporate a partial Fc without hinge but with a CH2 and CH3 domain, which can dimerize via the CH3 domain. In the embodiments hereinabove described in this paragraph, the remaining polypeptide components of the conjugate composition can be linked to either the N- or C-terminus of the targeting moiety, to enhance one or more properties of the resulting targeted conjugate composition.

“Antibody fragments” comprise a portion of an intact antibody or a synthetic or chimeric counterpart, preferably the antigen binding or variable region of the intact antibody. Examples of antibody fragments include molecules such as Fab fragments, Fab′ fragments, F(ab′)₂ fragments, Fd fragments, Fabc fragments, Fd fragments, Fabc fragments, domain antibodies (V_{HH}), single-chain antibody molecules (scFv), diabodies, individual antibody light chains, individual antibody heavy chains, chimeric fusions between antibody chains and other molecules, and the like.

A “Fab fragment” refers to a region of an antibody which binds to antigens. A Fab fragment is composed of a disulfide linked heterodimer of one constant and one variable domain of each of the heavy and the light chain These variable domains shape the paratope—the antigen binding site—at the amino terminal end of each monomer. Fab fragments can be generated in vitro. For example, the enzyme papain can be used to cleave an immunoglobulin monomer into two Fab fragments and an Fc fragment. The enzyme pepsin cleaves below the hinge region, so a F(ab′)₂ fragment and a Fc fragment is formed. As described more fully below, variable regions of the heavy and light chains can be fused together to form a single chain variable fragment (scFv), which retains the original specificity of the parent immunoglobulin.

The “light chains” of antibodies (immunoglobulins) from any vertebrate species can be assigned to one of two clearly distinct types, called kappa and lambda, based on the amino acid sequences of their constant domains.

The term “variable” refers to the fact that portions of the variable domains differ extensively in sequence among antibodies and confer the binding specificity of each particular antibody for its particular antigen. The variability is concentrated in three segments called complementarity-determining regions (CDRs) or hypervariable regions, both in the light-chain and the heavy-chain variable domains; i.e., LCDR1, LCDR2 and LCDR3, HCDR1, HCDR2 and HCDR3. In particular, the CDR regions from antibodies can be incorporated into targeting moieties of the subject compositions, but can be also be individually selected from one or more antibodies to create the binding domain. The more highly conserved portions of variable domains are called the framework regions (FR), which when combined with CDR sequences, may also be incorporated into targeting moieties. The variable domains of native heavy and light chains each comprise four FR regions, typically adopting a β-sheet configuration, connected by three CDRs that form loops. The CDRs in each chain are held together in close proximity by the FR regions and, with the CDRs from the other chain, contribute to the formation of the antigen-binding site of antibodies (see Kabat et al., NIH Publ. No. 91-3242, Vol. I, pages 647-669 (1991)). The constant domains are not involved directly in binding an antibody to an antigen, but exhibit or participate in various effector functions, such as antibody-dependent cellular toxicity.

Single-chain Variable Fragment Targeting Moieties

In one aspect, the present invention provides single-chain variable fragment binding fusion protein compositions. The term “single-chain variable fragment” or “scFv” means an antibody fragment that comprises one V_{H} and one V_{L} domain of an antibody, wherein these domains are present in a single polypeptide chain, and are generally joined by a polypeptide linker between the domains that enables the scFv to form the desired structure for antigen binding. Methods for making scFv's are known in the art (see, e.g., U.S. Pat. No. 6,806,079; Bird et al. (1988) Science 242:423-426; Huston et al. (1988) PNAS 85:5879-5883; Pluckthun in The Pharmacology of Monoclonal Antibodies, vol. 113, Rosenburg and Moore eds. Springer-Verlag, New York, pp. 269-315 (1994)). Two scFv can be combined in tandem in a single polypeptide to form a scFv-scFv fusion which can confer increased valency or specificity. Alternatively, two scFv can be joined non-covalently to form a diabody.

A binding domain of the scFv binding fusion protein compositions of the invention can have the N- to C-terminus configuration VH-linker-VL or VL-linker-VH. In one embodiment, the targeting moiety would then be fused to the CCD, PCM, and XTEN and optionally a second XTEN and PCM sequence linked to the N- or C-terminus of the resulting fusion protein, having at least the following structure permutations (N- to C-terminus); XTEN-PCM-CCD-VH-linker-VL; VH-linker-VL-CCD-PCM-XTEN; XTEN-PCM-CCD-VH-linker-VL-PCM-XTEN; XTEN-PCM-CCD-VL-linker-VH; VL-linker-VH-CCD-PCM-XTEN; XTEN-PCM-CCD-VL-linker-VH-CCD-PCM-XTEN. In another embodiment, two identical or distinct scFv in any format above can be joined. In another embodiment, the scFv would be conjugated to an XTEN, either at the N-terminus of the XTEN or to one or more cyteine or lysine residues of the XTEN. In the foregoing embodiment of the fusion proteins, the long carrier XTEN can comprise a sequence that can be a fragment of or that exhibits at least about 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from any one of Tables 10. In the foregoing embodiments of the scFv, the invention contemplates and encompasses compositions in which the VL and VH chains from the named antibodies, whether described in a narrative fashion or listed in the various tables, including Table 19, are incorporated into scFv linked by an appropriate linker, such as the sequence GSGEGSEGEGGGEGSEGEGSGEGGEGEGSG (SEQ ID NO: 590), or a sequence of Table 20 wherein the scFv can serve as a component to be either recombinantly fused to the CCD-PCM-XTEN fusion protein or PCM or is chemically conjugated as a component of a conjugate composition. In one embodiment, the invention provides a scFv TM for a conjugate composition in which the TM is derived from a monoclonal antibody of Table 19, wherein the corresponding VL and VL sequences have at least about 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to the VL and VH sequences of such monoclonal antibody. In another embodiment, the invention provides a scFv TM for an targeted conjugate composition in which the TM is derived from the VH and VL sequences listed for a monoclonal antibody of Table 19, wherein the VL and VL sequences of the TM have at least about 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to the VL and VH sequences of such monoclonal antibody and the VL and VH sequences would be linked by a linker sequence of Table 20 or a linker known in the art for svFv compositions, to result in the scFv.

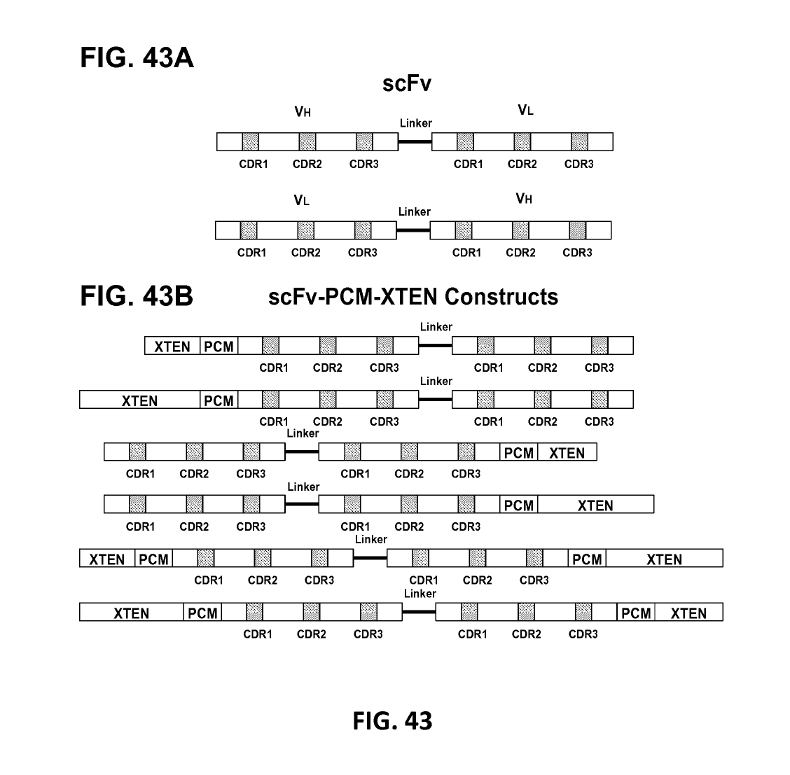

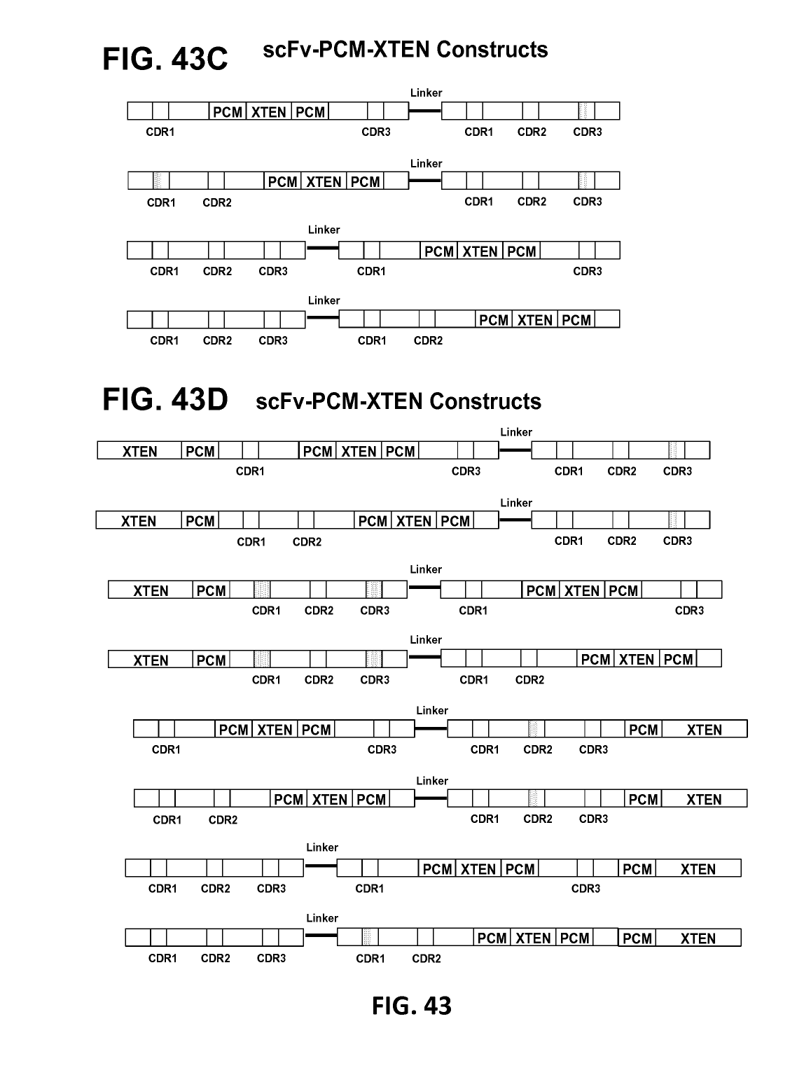

The invention also encompasses scFv targeting moieties constructed using fewer than the six CDRs found in a conventional antibody or scFv. In one embodiment, the scFv comprises five, four, or three CDR regions amongst the possible permutations of LCDR1, LCDR2 and LCDR3, HCDR1, and HCDR2 and HCDR3, intersperced with appropriate linkers, described below. Representative configurations of such scFv permutations are shown in FIG. 43. In one embodiment, the invention provides a scFv TM for an targeted conjugate composition in which the TM is derived from a monoclonal antibody of Table 19, wherein the corresponding LCDR1, LCDR2 and LCDR3, HCDR1, and HCDR2 and HCDR3 sequences have at least about 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to the LCDR1, LCDR2 and LCDR3, HCDR1, and HCDR2 and HCDR3 sequences of such monoclonal antibody. In another embodiment, the invention provides a scFv TM for an targeted conjugate composition in which the TM is derived from the LCDR1, LCDR2 and LCDR3, HCDR1, and HCDR2 and HCDR3 sequences of the VH and VL sequences listed for a monoclonal antibody of Table 19, wherein the LCDR1, LCDR2 and LCDR3, HCDR1, and HCDR2 and HCDR3 sequences of the TM have at least about 80% sequence identity, or 81%, 82%, 83%, 84%, 85%, 86%, 87%, 88%, 89%, 90%, 91%, 92%, 93%, 94%, 95%, 96%, 97%, 98%, 99%, or 100% sequence identity to the LCDR1, LCDR2 and LCDR3, HCDR1, and HCDR2 and HCDR3 sequences of such monoclonal antibody.

The linkers utilized to join the components of the targeting moieties are preferably flexible in nature. In one embodiment the linker joining the V_{L} and V_{H} binding domains that form the antigen binding site of the scFv targeting moiety can have from about 1 to about 30 amino acid residues in length. In another embodiment, the linker can have from about 30 to about 200 amino acid residues, or about 40 to about 144 amino acid residues, or about 50 to about 96 amino acid residues. In any of the embodiments hereinabove described in connection with targeting moieties, the linker can be a sequence derived from an XTEN sequence or a linker sequence of Table 20. In another embodiment, the linker can be a sequence in which at least 80% of the residues are comprised of amino acids glycine, serine, and/or glutamate, such as, but not limited to a sequence with about 80-100% sequence identify to the sequence GSGEGSEGEGGGEGSEGEGSGEGGEGEGSG (SEQ ID NO: 590), or a portion or a multimer thereof.

In one embodiment, the invention provides conjugate compositions comprising two or more scFv targeting moieties. In one embodiment, the two or more scFv targeting moieties may be identical. In another embodiment, the two or more scFv targeting moieties may be different and may bind to different targets (e.g., two or more targets of Tables 18-19) or to different epitopes on the same target. In the foregoing embodiments, the two or more scFv targeting moieties can be joined by a linker sequence, which can include a fragment of an XTEN sequence or a linker sequence of Table 20.

2. Proteins, Hormones and Organic Molecules as Targeting Moieties

In another aspect, the invention provides targeted conjugate compositions comprising XTEN covalently linked to non-antibody molecules that serves as a targeting moiety, which may be proteins, peptides, hormones, non-proteinaceous molecules, or organic molecules with specific binding affinity to a ligand from a target tissue or cell. In one embodiment, the non-antibody targeting moiety is a ligand to a cell surface receptor expressed on a cancer cell. In another embodiment, the non-antibody targeting moiety is a ligand to a cell surface receptor expressed on an inflammatory cell. In another embodiment, the non-antibody targeting moiety is a ligand to a luteinizing hormone-releasing hormone receptor expressed on a cancer cell. In another embodiment, the targeting moiety is one or more molecules of luteinizing hormone-releasing hormone, which targets a cancer cell. In another embodiment, the non-antibody targeting moiety is a ligand to a folate receptor expressed on a cancer cell. In another embodiment, the targeting moiety of the targeted conjugate composition is one or more molecules of folate, which targets a cancer cell. In another embodiment, the targeting moiety of the targeted conjugate compositions is one or more molecules of CTLA4. In another embodiment, the targeting moiety of the targeted conjugate compositions is one or more molecules of asparaginylglycylarginine (NGR) or an analog thereof. In another embodiment, the targeting moiety of the targeted conjugate compositions is one or more molecules of arginylglycylaspartic acid (RGD) or an analog thereof.

“Luteinizing hormone-releasing hormone” or “LHRH” means the human protein (UniProt No. P01148) encoded by the GNRH1 gene that is processed in the preoptic anterior hypothalamus from a 92-amino acid preprohormone into the linear decapeptide end-product having the sequence pyroGlu-His-Trp-Ser-Tyr-Gly-Leu-Arg-Pro-Gly-NH2 (SEQ ID NO: 600), as well as species and synthetic variations thereof, having at least a portion of the biological activity of the native peptide. LHRH plays a pivotal role in the regulation of the pituitary/gonadal axis, and thus reproduction. LHRH exerts its effects through binding to high-affinity receptors on the pituitary gonadotroph cells and subsequent release of FSH and LH. LHRH is found in organs outside of the hypothalamus and pituitary, and because a high percentage of certain cancer tissues have LHRH binding sites and because sex steroids have been implicated in the development of breast and prostate cancers, hormonal therapy with LHRH agonists are approved or are considered for the treatment of sex-steroid-dependent conditions such as estrogen-dependent breast cancer, ovarian cancer, endometrial cancer, bladder cancer and androgen-dependent prostate carcinoma. Because the half-life is reported to be less than 4 minutes. (Redding TW, et al. The Half-life, Metabolism and Excretion of Tritiated Luteinizing Hormone-Releasing Hormone (LH-RH) in Man. J Clin Endocrinol, Metab. (1973) 37:626-631). Accordingly, the invention contemplates use of LHRH as a selective targeting moiety in targeted conjugate compositions useful in treating cancers, described above.

In particular embodiments, the invention provides targeted conjugate compositions comprising one or more LHRH targeting components selected from Table 22 and one or more drug components selected from Tables 14-17. In the foregoing embodiment, the LHRH can be linked to a first XTEN that, in turn, is linked to one or more XTEN to which the drug components are conjugated, using the various configuration embodiments described herein. Alternatively, the LHRH and drug components can be conjugated to a monomeric XTEN.

TABLE 22
Exemplary LHRH
Composition

pGlu-HWSYGLRPG-NH2 (SEQ ID NO: 600)
pGlu-HWSY[D-Lys]LRPG-NH2
pGlu-HWSY[D-Trp]LRPG-NH2
pGlu-HWSY[D-Leu]LRP-NHEt
pGlu-HWSY[D-Ser(tBu)]LRP-NHEt
pGlu-HWSY[D-2-Nal]LRPG-NH2
pGlu-HWSY[D-His(Bzl)]LRP-NHEt
pGlu-HWSY[D-Ser(tBu)]LRP-Azagly-NH2
pGlu-HWSY[D-Trp]LRP-NHEt
pGlu-HWSHDWLPG-NH2 (SEQ ID NO: 601)

“Folate” and “folic acid” are used interchangeably herein to mean the chemical also known as pteroyl-L-glutamic acid, vitamin B9, folacin. and (2S)-2-[(4-{[(2-amino-4-hydroxypteridin-6-yl)methyl]amino}phenyl)formamido]pentanedioic acid. Folate is a ligand for the cell receptor known as folate receptor. Folate receptor alpha is a protein that in humans is encoded by the FOLR1 gene (Campbell IG, et al. (1991). Folate-binding protein is a marker for ovarian cancer (Cancer Res 51 (19): 5329-5338). Many cancer cells have a high requirement for folic acid and overexpress the folate receptor. The folate receptor encoded by this gene is a member of the folate receptor (FOLR) family, and members have a high affinity for folic acid and for several reduced folic acid derivatives, and mediate delivery of 5-methyltetrahydrofolate to the interior of cells. Folate receptor can be overexpressed by a number of tumors including ovarian, breast, renal, lung, colorectal, and brain. Accordingly, the invention contemplates use of folate as a selective targeting moiety in targeted conjugate compositions useful in treating cancers, described above.

“Arginylglycylaspartic acid” or “RGD” are used interchangeably herein to mean a tripeptide composed of L-arginine, glycine, and L-aspartic acid. RGD is a tripeptide sequence common in cellular recognition, and are ligands of integrins. RGD containing peptides can act as inhibitors of integrin-ligand interactions and induce apoptosis RGD peptides can interact with the tumor marker integrin alphaVbeta3, which is known to control angiogenesis, cell proliferation, and cell migration (Mol. Pharmaceutics (2012) 9:2961-2973). Integrin alphaVbeta3, a vitronectin receptor, has been implicated in several malignant tumors, including melanoma, glioma, ovarian, prostate, and breast cancer. Additionally, nearly all breast cancer tumors with a bone metastasis have high expression of integrin alphaVbeta3. Accordingly, the invention contemplates use of RGD as a selective targeting moiety in targeted conjugates conjugates useful in treating cancers. Exemplary RGD analogs useful as targeting moieties in the targeted conjugate compositions include RGDc, cRGC, cyclic(RGDyK), cyclic(RGDfK), cyclic(RGDfC), cyclic(RGDRf(N-Me)v), and cyclic(CGisoDGRG) (SEQ ID NO: 602). The invention also contemplates compositions in which the foregoing RGD analogs are incorporated in short XTEN fragments as targeting moieties.

“Asparaginylglycylarginine” or “NGR” are used interchangeably herein to mean a tripeptide of asparagine, glycine, and arginine NGR is a tripeptide sequence selected by phage display that specifically targets tumor vasculature by recognizing aminopeptidase N (APN or CD13) receptor on the cell membrane of tumor cells. Upon binding to APN, NGR peptides are internalized into cells via the endosomal pathway. Though APN is not exclusively expressed in tumor neovasculature, NGR peptides specifically target APN expressed in tumor blood vessels rather than other APN-expressing tissue (Cancer Res. (2002) 62:867-874). Increased APN expression has been noted for several malignant tumors, including breast, colon, non-small-cell lung, and pancreatic cancer (Cancer Sci. (2011) 102:501-508). Additionally, many cases of high APN tumor expression are correlated with poor survival. Accordingly, the invention contemplates use of NOR as a selective targeting moiety in targeted conjugates conjugates useful in treating cancers. Exemplary NOR analogs useful as targeting moieties in the targeted conjugate compositions include NGR, GNGRG (SEQ ID NO: 603), cyclic(NGR), cyclic(kNGRE), and CNGRC (cyclic disulfide) (SEQ ID NO: 604). The invention also contemplates compositions in which the foregoing NGR analogs are incorporated in short XTEN fragments as targeting moieties.

### VI). XTEN-Cross-Linker and Methods of Making Such Compositions

The present invention relates in part to highly purified preparations of XTEN-cross-linker conjugate compositions useful as conjugation partners to which payloads are conjugated, as described herein. The invention also relates to highly purified preparations of payloads linked to one or more XTEN using the XTEN-cross-linker conjugation partners. The present invention encompasses compositions and methods of making the targeted conjugate compositions formed by linking of any of the herein described XTEN with a payload, as well as reactive compositions and methods of making the compositions formed by conjugating XTEN with a cross-linker or other chemical methods described herein. It is specifically intended that the terms “CCD-conjugate”, “CCD-cross-linker”, “XTEN-conjugate” and “XTEN-cross-linker” encompass the linked reaction products remaining after the conjugation of the reactant conjugation partners, including the reaction products of cross-linkers, click-chemistry reactants, or other methods described herein.

In some embodiments, the CCD and XTEN utilized to create the subject conjugates comprise one or more CCD or XTEN selected from any one of the sequences in Table5, Table 10, or Table 11, which may be linked to the payload component directly or via cross-linkers disclosed herein. In one embodiment, the CCD utilized to create the targeted conjugate compositions comprise a CCD having at least 90%, 91%, 92%, 93%, 94%, 95%, 96%, 97%, 98%, 99%, or 100% sequence identity compared to a CCD sequence selected from Table 5. In other embodiments, the one or more XTEN utilized to create the subject conjugates individually comprise an XTEN sequence having at least about 80% sequence identity, or alternatively 90%, 91%, 92%, 93%, 94%, 95%, 96%, 97%, 98%, 99%, or 100% sequence identity compared to an XTEN selected from Table 10 or Table 11 or a fragment thereof, when optimally aligned with a sequence of comparable length. In one embodiment, the subject conjugates are multimeric in that they comprise a first and a second XTEN sequence, wherein the XTEN are the same or they are different and wherein each individually comprises an XTEN sequence having at least about 90% sequence identity, or alternatively 91%, 92%, 93%, 94%, 95%, 96%, 97%, 98%, 99%, or 100% sequence identity compared to an XTEN selected from Table 10 or Table 11 or a fragment thereof, when optimally aligned with a sequence of comparable length. In another embodiment, the subject conjugates are multimeric in that they comprise a first, a second, or a third XTEN sequences, wherein the XTEN are the same or they are different and wherein each individually comprises an XTEN sequence having at least about 90% sequence identity, or alternatively 91%, 92%, 93%, 94%, 95%, 96%, 97%, 98%, 99%, or 100% sequence identity compared to an XTEN selected from Table 10 or Table 11 or a fragment thereof, when optimally aligned with a sequence of comparable length. In yet another embodiment, the subject conjugates are multimeric in that they comprise 3, 4, 5, 6 or more XTEN sequences, wherein the XTEN are the same or they are different and wherein each individually comprises an XTEN sequence having at least about 80% sequence identity, or alternatively 81%, 82%, 83%, 84%, 85%, 86%, 87%, 88%, 89%, 90%, 91%, 92%, 93%, 94%, 95%, 96%, 97%, 98%, 99%, or 100% sequence identity compared to an XTEN selected from Table 10 or Table 11 or a fragment thereof. In the multimeric conjugates, the cumulative length of the residues in the XTEN sequences is greater than about 100 to about 3000, or about 400 to about 1000 amino acid residues, and the XTEN can be identical or they can be different in sequence or in length. As used herein, cumulative length is intended to encompass the total length, in amino acid residues, when more than one XTEN is incorporated into the conjugate.

The present invention encompasses compositions and methods of making CCD and/or XTEN covalently linked to a small molecule payload drugs, resulting in a conjugate, as well as compositions of CCD or XTEN covalently linked to a payload biologically active proteins (which encompasses peptides or polypeptides), that, along with the other components (e.g., targeting moiety and PCM) result in a targete conjugate composition. In another aspect, the invention provides compositions of one or more CCD or XTEN linked to payloads of one or more drugs, one or more targeting moieties, and one or more peptidyl cleavage moities (PCM) resulting in the targeted conjugate compositions of the instant invention. In particular, the invention provides such targeted conjugate compositions useful in the treatment of a disease or condition for which the administration of a payload drug and/or protein that is useful in the treatment, amelioration or prevention of a disease or condition in a subject. The targeted conjugate compositions of some embodiments generally comprise one or more of the following components: 1) XTEN; 2) CCD; 3) cross-linker; 4) payload, 5) targeting moiety, and, optionally, 5) PCM to which the components are recombinantly fused or chemically conjugated; either directly or by use of a cross-linker, such as commercially-available cross-linkers described herein, or by use of click-chemistry reactants, or in some cases, may be created by conjugation between reactive groups in the CCD or XTEN and payload without the use of a linker as described herein. However, in some cases of foregoing types of compositions, the composition can be created without the use of a cross-linker provided the components are otherwise chemically reactive.

The conjugation of CCD or XTEN to payloads and targeting moieties confers several advantages on the resulting compositions compared to the payloads not linked to CCD or XTEN. As described more fully below, non-limiting examples of the enhanced properties include increases in the overall solubility and metabolic stability, reduced susceptibility to proteolysis in circulation, reduced immunogenicity, reduced rate of absorption when administered subcutaneously or intramuscularly, reduced clearance by the kidney, enhanced interactions with the target tissues by virtue of the targeting moiety with concommitant reduced toxicity, targeted delivery of payload, reduced toxicity of the payload component by virtue of the shielding effect of XTEN until released by cleavage of the PCM, and enhanced pharmacokinetic properties. In particular, it is specifically contemplated that the subject compositions, in accordance with some embodiments, are designed such that they have an enhanced therapeutic index and reduced toxicity or side effects, achieved by a combination of the shielding effect and steric hindrence of XTEN together with targeted delivery (achieved by inclusion of a targeting moiety in the composition) and release of the payload (achieved by inclusion of a peptidyl cleave moiety in the composition) in proximity to or within a target tissue that produces a protease for with the peptidyl cleave moiety is a substrate. In addition, it is contemplated that the compositions will, by their design and linkage to XTEN, have enhanced pharmacokinetic properties compared to the corresponding payload(s) not linked to XTEN, e.g., a terminal half-life increased by at least about 2-fold, 3-fold, 4-fold, 5-fold, 6-fold, 7-fold, 8-fold, 9-fold, 10-fold, 20-fold, 30-fold, 40-fold, or 100-fold greater, increased area under the curve (AUC) (e.g., 25%, 50%, 100%, 200%, 300% or more), lower volume of distribution, slower absorption after subcutaneous or intramuscular injection (an advantage compared to commercially-available forms of payload that must be administered by a similar route) such that the Cmax is lower, which, in turn, results in reductions in adverse effects of the payload that, collectively, results in an increased period of time that a conjugation composition administered to a subject provides therapeutic activity. In some embodiments, the conjugation compositions comprise cleavage sequences (described more fully, above) that permits sustained release of active payload such that the administered targeted conjugate composition acts as a depot when subcutaneously or intramuscularly administered, even after entering the blood circulatory system. It is specifically contemplated that targeted conjugate compositions can exhibit one or more or any combination of the improved properties disclosed herein. As a result of these enhanced properties, the targeted conjugate compositions permit less frequent dosing, more tailored dosing, and/or reduced toxicity compared to payload not linked to the targeted conjugate composition and administered in a comparable fashion. Such targeted conjugate compositions have utility to treat certain conditions known in the art to be affected, ameliorated, or prevented by administration of the payload to a subject in need thereof, as described herein.

1. Cross-Linker Reactants for Conjugation

In another aspect, the invention relates to CCD or XTEN conjugated to cross-linkers, resulting in CCD-cross-linker and XTEN-cross-linker conjugates that can be utilized to prepare targeted conjugate compositions. In particular, the herein-described CCD-cross-linker and XTEN-cross-linker conjugate partners are useful for conjugation to payload agents bearing at least one thiol, amino, aminooxy, carboxyl, aldehyde, alcohol, azide, alkyne or any other reactive group available and suitable, as known in the art, for reaction between the components described herein.

In another aspect, the invention relates to payloads conjugated to cross-linkers, resulting in payload-cross-linker conjugates that can be utilized to prepare targeted conjugate compositions. In particular, the herein-described payload-cross linker partners are useful for conjugation to CCD or XTEN bearing at least one thiol, amino, aminooxy, carboxyl, aldehyde, alcohol, azide, alkyne or any other reactive group available and suitable, as known in the art, for reaction between the components described herein.

Exemplary embodiments of CCD and XTEN have been described above, including preparations of substantially homogeneous XTEN. The invention provides CCD and XTEN that further serve as a platform to which payloads can be conjugated, such that they serve as a “carrier”, conferring certain desirable pharmacokinetic, chemical and pharmaceutical properties to the compositions, amongst other properties described below. In other embodiments, the invention provides polynucleotides that encode CCD or XTEN that can be linked to genes encoding peptide or polypeptide payloads that can be incorporated into expression vectors and incorporated into suitable hosts for the expression and recovery of the subject recombinant fusion proteins.

In some embodiments, the CCD or XTEN components as described herein, above, are engineered to incorporate a defined number of reactive amino acid residues that can be reacted with cross-linking agents or can further contain reactive groups that can be used to conjugate to payloads. In one embodiment, the invention provides CCD comprising one or more a cysteine residues wherein the cysteine, each of which contains a reactive thiol group, are conjugated to a cross-linker, resulting in a CCD-cross-linker conjugate or to thiol-reactive payload, resulting in CCD-payload conjugate. In another embodiment, the invention provides a cysteine-engineered XTEN, such as the sequences of Table 11, wherein the cysteine, each of which contains a reactive thiol group, are conjugated to a cross-linker, resulting in an XTEN-cross-linker conjugate or to thiol-reactive payload, resulting in a XTEN-payload conjugate. In another embodiment, invention provides XTEN with α-amino group or lysine-engineered XTEN wherein lysine, each of which contains a positively charged hydrophilic ε-amino group, are conjugated to a cross-linker, resulting in an XTEN-cross-linker conjugate or to amine-reactive payload, resulting in an XTEN-payload conjugate. In the embodiments of cysteine-engineered XTEN, each comprises about 1 to about 100 cysteine amino acids, or from 1 to about 50 cysteine amino acids, or from 1 to about 40 cysteine amino acids, or from 1 to about 20 cysteine amino acids, or from 1 to about 10 cysteine amino acids, or from 1 to about 5 cysteine amino acids, or 9 cysteines, or 3 cysteines, or a single cysteine amino acid that is available for conjugation. In the embodiments of lysine-engineered XTEN, each comprises about 1 to about 100 lysine amino acids, or from 1 to about 50 lysine amino acids, or from 1 to about 40 lysine engineered amino acids, or from 1 to about 20 lysine engineered amino acids, or from 1 to about 10 lysine engineered amino acids, or from 1 to about 5 lysine engineered amino acids, or a single lysine that is available for conjugation. In another embodiment, the engineered XTEN comprises both cysteine and lysine residues of the foregoing ranges or numbers. In another embodiment, the invention provides CCD wherein each comprises about 1 to about 10 cysteine amino acids, or from 1 to about 10 cysteine amino acids, or from 1 to about 3 cysteine amino acids. In one embodiment, the invention provides CCD wherein the incorporated cysteine, each of which contains a reactive thiol group, are conjugated to a cross-linker, resulting in an CCD-cross-linker conjugate.

Generally, cysteine thiol groups are more reactive (specifically, more nucleophilic) towards electrophilic conjugation reagents than amine or hydroxyl groups. In addition, cysteine residues are generally found in smaller numbers in a given protein; thus are less likely to result in multiple conjugations within the same protein. Cysteine residues have been introduced into proteins by genetic engineering techniques to form covalent attachments to ligands or to form new intramolecular disulfide bonds (Better et al (1994) J. Biol. Chem. 13:9644-9650; Bernhard et al (1994) Bioconjugate Chem. 5:126-132; Greenwood et al (1994) Therapeutic Immunology 1:247-255; Tu et al (1999) Proc. Natl. Acad. Sci USA 96:4862-4867; Kanno et al (2000) J. of Biotechnology, 76:207-214; Chmura et al (2001) Proc. Nat. Acad. Sci. USA 98(15):8480-8484; U.S. Pat. No. 6,248,564).

In one embodiment, the invention provides an isolated composition comprising a cysteine-engineered XTEN or CCD conjugated to a cross-linker, wherein the cross-linker is selected from sulfhydryl-reactive homobifunctional or heterobifunctional cross-linkers. In another embodiment, the invention provides an isolated composition comprising a lysine-engineered XTEN conjugated by a cross-linker, wherein the cross-linker is selected from amine-reactive homobifunctional or heterobifunctional cross-linkers. Cross-linking generally refers to a process of chemically linking two or more molecules by a covalent bond. The process is also called conjugation or bioconjugation with reference to its use with proteins and other biomolecules. For example, proteins can be modified to alter N- and C-termini, and amino acid side chains on proteins and peptides in order to block or expose reactive binding sites, inactivate functions, or change functional groups to create new targets for cross-linking

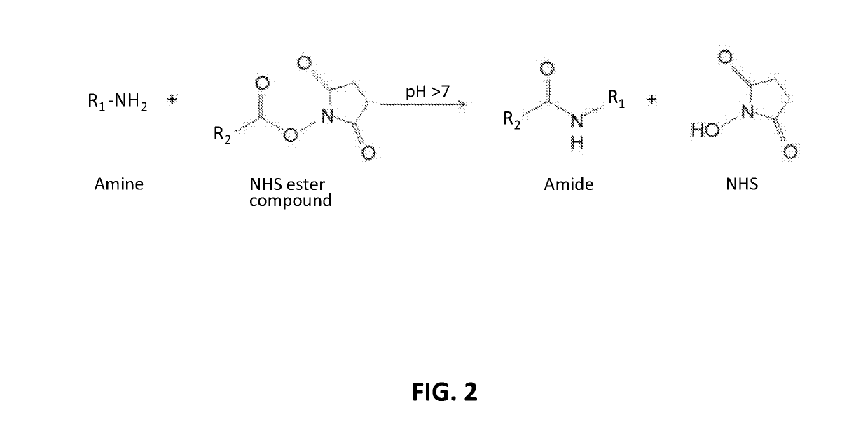

In one aspect, the invention provides methods for the site-specific conjugation to XTEN polymer, accomplished using chemically-active amino acid residues or their derivatives (e.g., the N-terminal α-amine group, the ε-amine group of lysine, the thiol group of cysteine, the C-terminal carboxyl group, carboxyl groups of glutamic acid and aspartic acid). Functional groups suitable for reactions with primary α- and ε-amino groups are chlorocyanurates, dichlorotreazines, trezylates, benzotriazole carbonates, p-nitrophenyl carbonates, trichlorophenyl carbonates, aldehydes, mixed anhydrides, carbonylimidazoles, imidoesters, tetrafluorophenyl (TFP) and pentafluorophenyl (PFP) esters, N-hydroxysuccinimide esters, N-hydroxysulfosuccinimide esters (Harris, J. M., Herati, R. S. Polym. Prepr. (Am. Chem. Soc., Div. Polym. Chem), 32(1), 154-155 (1991); Herman, S., et al. Macromol. Chem. Phys. 195, 203-209 (1994); Roberts, M. J. et. al. Advanced Drug Delivery Reviews, 54, 459-476 (2002)). N-hydroxysuccinimide esters (NHS-esters and their water soluble analogs sulfo-NHS-esters) are commonly used for protein conjugation (see FIG. 2). NHS-esters yield stable amide products upon reaction with primary amines with relatively efficient coupling at physiological pH. The conjugation reactions are typically performed in 50-200 mM phosphate, bicarbonate/carbonate, HEPES or borate buffers (pH between 7 and 9) at 4° C. to room temperature from 0.5 to 2 hrs. NHS-esters are usually used at two- to 50-fold molar excess to protein. Typically, the concentration of the reagent can vary from 0.1-10 mM, while the optimal protein concentration is 50-100 μM.

In another method, given that XTEN polypeptides possess only a single N-terminal α-amino group, the XTEN can be engineered to contain additional α-amino group(s) of intentionally incorporated lysine residues; exemplary sequences of which are provided in Table 11. The α-and ε-amino groups have different pKa values: approximately 7.6 to 8.0 for the α-amino group of the N-terminal amino acid, and approximately 10-10.5 for the ε-amino group of lysine. Such a significant difference in pKa values can be used for selective modification of amino groups. Deprotonation of all primary amines occurs at pH above pH 8.0. In this environment, the nucleophilic properties of different amines determine their reactivity. When deprotonated, the more nucleophilic ε-amino groups of lysines are generally more reactive toward electrophiles than α-amino groups. On the other hand, at a lower pH (for example pH 6), the more acidic α-amino groups are generally more deprotonated than ε-amino groups, and the order of reactivity is inverted. For example, the FDA-approved drug Neulasta (pegfilgranstim) is granulocyte colony-stimulating factor (G-CSF) modified by covalent attachment of 20 kDa PEG-aldehyde. Specific modification of the protein's N-terminal amino acid was accomplished by exploiting the lower pKa of α-amino group as compared to ε-amino groups of internal lysines (Molineaux, G. Curr. Pharm. Des. 10, 1235-1244 (2004), U.S. Pat. No. 5,824,784).

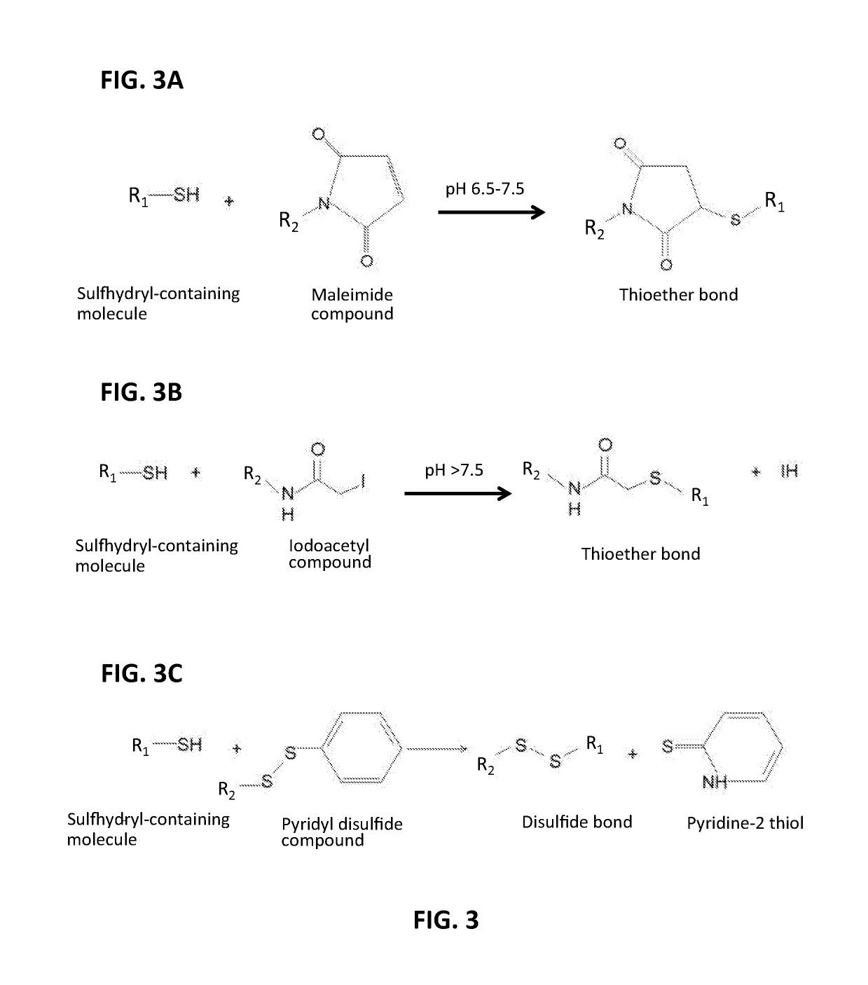

The CCD and XTEN polypeptides comprising cysteine residues can be genetically engineered using recombinant methods described herein (see, e.g., Examples) or by standard methods known in the art. Conjugation to thiol groups can be carried using highly specific reactions, leading to the formation of single conjugate species joined by cross-linking agents. Functional groups suitable for reactions with cysteine thiol-groups are N-maleimides, haloacetyls, and pyridyl disulfides. The maleimide group reacts specifically with sulfhydryl groups when the pH of the reaction mixture is between pH 6.5 and 7.5, forming a stable thioether linkage that is not reversible (see FIG. 3). At neutral pH, maleimides react with sulfhydryls 1,000-fold faster than with amines, but when the pH is raised to greater than 8.5, the reaction favors primary amines Maleimides do not react with tyrosines, histidines or methionines. For reaction solutions, thiols must be excluded from reaction buffers used with maleimides as they will compete for coupling sites. Excess maleimides in the reaction can be quenched at the end of a reaction by adding free thiols, while EDTA can be included in the coupling buffer to minimize oxidation of sulfhydryls.

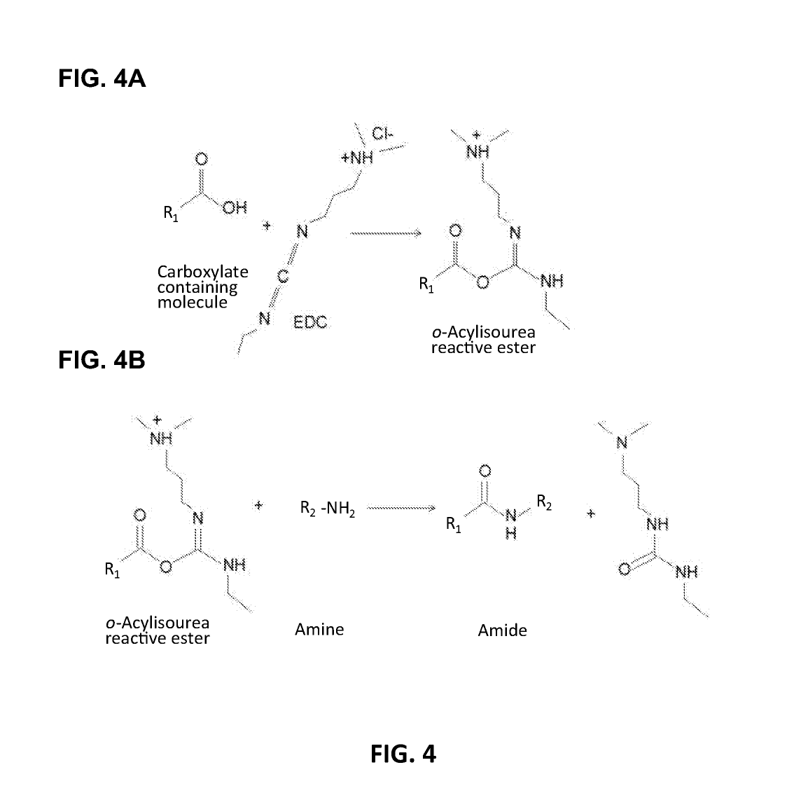

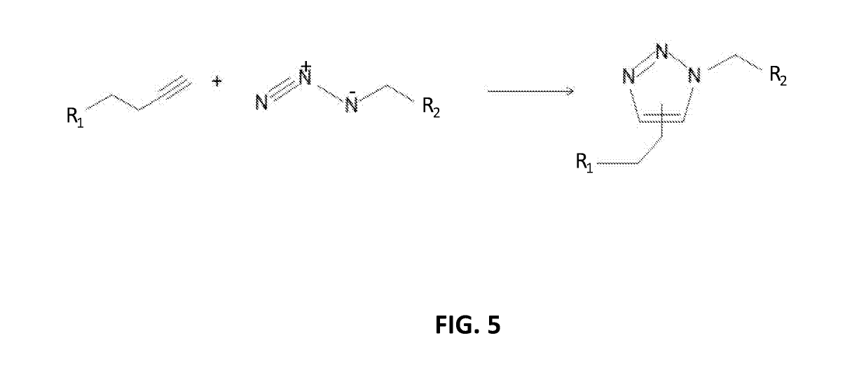

In another embodiment, the invention contemplates use of haloacetyl reagents that are useful for cross-linking sulfhydryls groups of CCD or XTEN or payloads to prepare the subject conjugates. The most commonly used haloacetyl reagents contain an iodoacetyl group that reacts with sulfhydryl groups at physiological pH. The reaction of the iodoacetyl group with a sulfhydryl proceeds by nucleophilic substitution of iodine with a thiol producing a stable thioether linkage (see FIG. 4). Using a slight excess of the iodoacetyl group over the number of sulfhydryl groups at pH 8.3 ensures sulfhydryl selectivity. If a large excess of iodoacetyl group is used, the iodoacetyl group can react with other amino acids. Imidazoles can react with iodoacetyl groups at pH 6.9-7.0, but the incubation must typically proceed for longer than one week. Histidyl side chains and amino groups react in the unprotonated form with iodoacetyl groups above pH 5 and pH 7, respectively. In another embodiment, cross-linkers useful for sulfhydryls groups are pyridyl disulfides. Pyridyl disulfides react with sulfhydryl groups over a broad pH range (the optimal pH is 4-5) to form disulfide bonds linking CCD or XTEN to payloads (see FIG. 5). As a disulfide, conjugates prepared using these reagents are cleavable. During the reaction, a disulfide exchange occurs between the molecule's —SH group and the 2-pyridyldithiol group. As a result, pyridine-2-thione is released. These reagents can be used as crosslinkers and to introduce sulfhydryl groups into proteins. The disulfide exchange can be performed at physiological pH, although the reaction rate is slower.

The targeted conjugate compositions comprising active synthetic peptides or polypeptides can be prepared using chemically active amino acid residues or their derivatives; e.g., the N-terminal α-amino group, the ε-amino group of lysine, a thiol group of cysteine, the carboxyl group of the C-terminal amino acid, a carboxyl group of aspartic acid or glutamic acid. Each peptide contains N-terminal α-amino group regardless of a primary amino acid sequence. If necessary, N-terminal α-amino group can be left protected/blocked upon chemical synthesis of the active peptide/polypeptide. The synthetic peptide/polypeptide may contain additional ε-amino group(s) of lysine that can be either natural or specifically substituted for conjugation.

Since cysteines are generally less abundant in natural peptide and protein sequences than lysines, the use of cysteines as a site for conjugation reduces the likelihood of multiple conjugations to XTEN-cross-linker molecules in a reaction. It also reduces the likelihood of peptide/protein deactivation upon conjugation. Moreoever, conjugation to cysteine sites can often be carried out in a well-defined manner, leading to the formation of single species polypeptide conjugates. In some cases cysteine may be absent in the amino acid sequence of the peptide to be conjugated. In such a case, cysteine residue can be added to the N- or C-terminus of the peptide either recombinantly or synthetically using standard methods. Alternatively, a selected amino acid can be chemically or genetically modified to cysteine. As one example, serine modification to cysteine is considered a conservative mutation. Another approach to introduce a thiol group in cysteine-lacking peptides is chemical modification of the lysine ε-amino group using thiolating reagents such as 2-iminothiolane (Traut's reagent), SATA (N-succinimidyl S-acetylthioacetate), SATP (N-succinimidyl 5-acetylthiopropionate), SAT-PEO₄-Ac (N-Succinimidyl S-acetyl(thiotetraethylene glycol)), SPDP (N-Succinimidyl 3-(2-pyridyldithio)propionate), LC-SPDP (Succinimidyl 6-(3′-[2-pyridyldithio]propionamido)hexanoate) (described more fully, below). Once a unique thiol group is introduced in the peptide, it can be selectively modified by compounds containing sufhydryl-reactive such as N-maleimides, haloacetyls, and pyridyl disulfides, as described above.

The conjugation between the CCD or XTEN polypeptide and a peptide, protein or small molecule drug payload may be achieved by a variety of linkage chemistries, including commercially available zero-length, homo- or hetero-bifunctional, and multifunctional cross-linker compounds, according to methods known and available in the art, such as those described, for example, in R. F. Taylor (1991) “Protein immobilization. Fundamentals and Applications”, Marcel Dekker Inc., N.Y.; G. T. Hermanson et al. (1992) “Immobilized Affinity Ligand Techniques”, Academic Press, San Diego; G. T. Hermanson (2008) “Bioconjugate Techniques”, 2^{nd}. ed. Elsevier, Inc., S. S. Wong (1991) “Chemistry of Protein Conjugation and Crosslinking”, CRC Press, Boca Raton. Suitable cross-linking agents for use in preparing the conjugates of the disclosure are commercially-available from companies like Sigma-Aldrich, Thermo Scientific (Pierce and Invitrogen Protein Research Products), ProteoChem, G-Biosciences. Preferred embodiments of cross-linkers comprise a thiol-reactive functional group or an amino-reactive functional group. A list of exemplary cross-linkers is provided in Table 23.

TABLE 23
Exemplary cross-linkers
Cross-linker

maleimides, haloacetyls, pyridyl disulfides, AMAS (N-(α-Maleimidoacetoxy)-succinimide ester),
BMB (1,4-Bis-Maleimidobutane), BMDB (1,4 Bismaleimidyl-2,3-dihydroxybutane), BMH (Bis-
Maleimidohexane), BMOE (Bis-Maleimidoethane), BMPH (N-(β-Maleimidopropionic
acid)hydrazide), BMPS (N-(β-Maleimidopropyloxy)succinimide ester), BM(PEG)₂ (1,8-Bis-
Maleimidodiethylene-glycol), BM(PEG)₃ (1,11-Bis-Maleimidotriethyleneglycol), BS²G (Bis
(sulfosuccinimidyl)glutarate), BS³ (Sulfo-DSS) (Bis (sulfosuccinimidyl)suberate), BS[PEG]₅ (Bis
(NHS)PEG5), BS(PEG)₉ (Bis (NHS)PEG9), BSOCOES (Bis(2-
[succinimidoxycarbonyloxy]ethyl)sulfone), C6-SANH (C6-Succinimidyl 4-hydrazinonicotinate
acetone hydrazone), C6-SFB (C6-Succinimidyl 4-formylbenzoate), DCC (N,N-
Dicyclohexylcarbodiimide), DPDPB (1,4-Di-(3′-[2′pyridyldithio]propionamido) butane), DSG
(Disuccinimidyl glutarate), DSP (Dithiobis(succimidylpropionate), Lomant's Reagent), DSS
(Disuccinimidyl suberate), DST (Disuccinimidyl tartarate), , DTME (Dithiobis-maleimidoethane),
DTSSP (Sulfo-DSP) (3,3′-Dithiobis (sulfosuccinimidylpropionate)), EDC (1-Ethyl-3-(3-
dimethylaminopropyl) carbodiimide hydrochloride), EGS (Ethylene glycol
bis(succinimidylsuccinate)), EMCA (N-ε-Maleimidocaproic acid), EMCH (N-(ε-Maleimidocaproic
acid)hydrazide), EMCS (N-(ε-Maleimidocaproyloxy)succinimide ester), GMBS (N-(γ-
Maleimidobutyryloxy)succinimide ester), KMUA (N-κ-Maleimidoundecanoic acid), KMUH (N-(κ-
Maleimidoundecanoic acid)hydrazide), LC-SMCC (Succinimidyl 4-(N-maleimidomethyl)
cyclohexane-1-carboxy-(6-amidocaproate)), LC-SPDP (Succinimidyl 6-(3′-[2-
pyridyldithio]propionamido)hexanoate), MBS (m-Maleimidobenzoyl-N-hydroxysuccinimide ester),
MPBH (4-(4-N-Maleimidophenyl)-butyric acid hydrazide), PDPH (3-(2-
Pyridyldithio)propionylhydrazide), SANH (Succinimidyl 4-hydrazinonicotinate acetone hydrazone),
SBAP (Succinimdyl 3-(bromoacetamido)propionate), , SFB (Succinimidyl 4-formylbenzoate), SHTH
(Succinimidyl 4-hydrazidoterephthalate), SIA (N-succinimidyl iodoacetate), SIAB (N-
Succinimidyl(4-iodoacetyl)aminobenzoate), SMPB (Succinimidyl 4-(p-maleimidophenyl) butyrate),
SMCC (Succinimidyl 4-(N-maleimido-methyl)cyclohexane-1-carboxylate), SM[PEG]₂ (NHS-PEG2-
Maliemide), SM[PEG]₄ (NHS-PEG4-Maliemide), SM(PEG)₆ (NHS-PEG6-Maleimide), SM[PEG]₈
(NHS-PEG8-Maliemide), SM[PEG]₁₂ (NHS-PEG12-Maliemide), SM(PEG)₂₄ (NHS-PEG24-
Maleimide), SMPB (Succinimidyl 4-(p-maleimido-phenyl)butyrate), SMPH (Succinimidyl-6-(β-
maleimidopropionamido)hexanoate), SMPT (4-Succinimidyloxycarbonyl-methyl-α-(2-
pyridyldithio)toluene), SPB (Succinimidyl-(4-psoralen-8-yloxy)butyrate), SPDP (N-Succinimidyl 3-
(2-pyridyldithio)propionate), Sulfo-DST (Sulfodisuccinimidyl tartrate), Sulfo-EGS (Ethylene glycol
bis (sulfo-succinimidyl succinate)), Sulfo-EMCS (N-(ε-Maleimidocaproyloxy)sulfosuccinimide
ester), Sulfo-GMBS (N-(γ-Maleimidobutryloxy)sulfosuccinimide ester), , Sulfo-KMUS (N-(κ-
Maleimidoundecanoyloxy)sulfosuccinimide ester), Sulfo-LC-SMPT (Sulfosuccinimidyl 6-(α-methyl-
α-[2-pyridyldithio]-toluamido)hexanoate), Sulfo-LC-SPDP (Sulfosuccinimidyl 6-(3′-[2-
pyridyldithio]propionamido)hexanoate), Sulfo-MBS (m-Maleimidobenzoyl-N-
hydroxysulfosuccinimide ester), Sulfo-SIA (N-sulfosuccinimidyl iodoacetate), Sulfo-SIAB
(Sulfosuccinimidyl(4-iodo-acetyl)aminobenzoate), Sulfo-SMCC (Sulfosuccinimidyl 4-(N-
maleimidomethyl)cyclohexane-1-carboxylate), Sulfo-SMPB (Sulfosuccinimidyl 4-(p-
maleimidophenyl)butyrate), TMEA (Tris-(2-Maleimidoethyl)amine), TSAT (Tris-(succinimidyl
aminotriacetate)), 3-propargyloxypropanoic acid NHS ester, acetylene-PEG-NHS ester,
dibenzylcyclooctyne (DBCO)-NHS ester, DBCO-PEG-NHS ester, cyclooctyne (COT)-NHS ester,
COT-PEG-NHS ester, COT-PEG-pentafluorophenyl (PFP) ester, BCOT-NHS ester, BCOT-PEG-
NHS ester, BCOT-PEG-pentafluorophenyl (PFP) ester, Acetylene-PEG4-maleimide, DBCO-
maleimide, COT-maleimide, BCOT-maleimide, 3-azide-propionic acid NHS ester, 6-azide-hexanoic
acid NHS ester, 3-azide-propionic acidPFP ester, 6-azide-hexanoic acid PFP ester, azide-PEG-NHS
ester, azide-PEG-PFP ester, azide-PEG-maleimide

Non-limiting examples of cross-linkers are BMB (1,4-Bis-Maleimidobutane), BMDB (1,4 Bismaleimidyl-2,3-dihydroxybutane), BMH (Bis-Maleimidohexane), BMOE (Bis-Maleimidoethane), BMPH (N-(β-Maleimidopropionic acid)hydrazide), BMPS (N-(β-Maleimidopropyloxy)succinimide ester), BM(PEG)₂ (1,8-Bis-Maleimidodiethylene-glycol), BM(PEG)₃ (1,11-Bis-Maleimidotriethyleneglycol), BS²G (Bis (sulfosuccinimidyl)glutarate), BS³ (Sulfo-DSS) (Bis (sulfosuccinimidyl)suberate), BS[PEG]₅ (Bis (NHS)PEG5), BS(PEG)₉ (Bis (NHS)PEG9), BSOCOES (Bis(2-[succinimidoxycarbonyloxy]ethyl)sulfone), C6-SANH (C6-Succinimidyl 4-hydrazinonicotinate acetone hydrazone), C6-SFB (C6-Succinimidyl 4-formylbenzoate), DCC (N,N-Dicyclohexylcarbodiimide), DPDPB (1,4-Di-(3′-[2′pyridyldithio]propionamido) butane), DSG (Disuccinimidyl glutarate), DSP (Dithiobis(succimidylpropionate), Lomant's Reagent), DSS (Disuccinimidyl suberate), DST (Disuccinimidyl tartarate), DTME (Dithiobis-maleimidoethane), DTSSP (Sulfo-DSP) (3,3′-Dithiobis (sulfosuccinimidylpropionate)), EDC (1-Ethyl-3-(3-dimethylaminopropyl) carbodiimide hydrochloride), EGS (Ethylene glycol bis(succinimidylsuccinate)), EMCA (N-E-Maleimidocaproic acid), EMCH (N-(ε-Maleimidocaproic acid)hydrazide), EMCS (N-(E-Maleimidocaproyloxy)succinimide ester), GMBS (N-(γ-Maleimidobutyryloxy)succinimide ester), KMUA (N-κ-Maleimidoundecanoic acid), KMUH (N-(κ-Maleimidoundecanoic acid)hydrazide), LC-SMCC (Succinimidyl 4-(N-maleimidomethyl) cyclohexane-1-carboxy-(6-amidocaproate)), LC-SPDP (Succinimidyl 6-(3′-[2-pyridyldithio]propionamido)hexanoate), MBS (m-Maleimidobenzoyl-N-hydroxysuccinimide ester), MPBH (4-(4-N-Maleimidophenyl)-butyric acid hydrazide), SBAP (Succinimdyl 3-(bromoacetamido)propionate), SFB (Succinimidyl 4-formylbenzoate), SHTH (Succinimidyl 4-hydrazidoterephthalate), SIA (N-succinimidyl iodoacetate), SIAB (N-Succinimidyl(4-iodoacetyl)aminobenzoate), SMPB (Succinimidyl 4-(p-maleimidophenyl) butyrate), SMCC (Succinimidyl 4-(N-maleimido-methypcyclohexane-1-carboxylate), SM[PEG]₂ (NHS-PEG2-Maliemide), SM[PEG]₄ (NHS-PEG4-Maliemide), SM(PEG)₆ (NHS-PEG6-Maleimide), SM[PEG]₈ (NHS-PEG8-Maliemide), SM[PEG]₁₂ (NHS-PEG12-Maliemide), SM(PEG)₂₄ (NHS-PEG24-Maleimide), SMPB (Succinimidyl 4-(p-maleimido-phenyl)butyrate), SMPH (Succinimidyl-6-(β-maleimidopropionamido)hexanoate), SMPT (4-Succinimidyloxycarbonyl-methyl-α-(2-pyridyldithio)toluene), SPB (Succinimidyl-(4-psoralen-8-yloxy)butyrate), SPDP (N-Succinimidyl 3-(2-pyridyldithio)propionate), Sulfo-DST (Sulfodisuccinimidyl tartrate), Sulfo-EGS (Ethylene glycol bis (sulfo-succinimidyl succinate)), Sulfo-EMCS (N-(ε-Maleimidocaproyloxy)sulfosuccinimide ester), Sulfo-GMBS (N-(γ-Maleimidobutryloxy)sulfosuccinimide ester), Sulfo-KMUS (N-(κ-Maleimidoundecanoyloxy)sulfosuccinimide ester), Sulfo-LC-SMPT (Sulfosuccinimidyl 6-(α-methyl-α-[2-pyridyldithio]-toluamido)hexanoate), Sulfo-LC-SPDP (Sulfosuccinimidyl 6-(3′-[2-pyridyldithio]propionamido)hexanoate), Sulfo-MBS (m-Maleimidobenzoyl-N-hydroxysulfosuccinimide ester), Sulfo-SIAB (Sulfosuccinimidyl(4-iodo-acetyl)aminobenzoate), Sulfo-SMCC (Sulfosuccinimidyl 4-(N-maleimidomethyl)cyclohexane-1-carboxylate), Sulfo-SMPB (Sulfosuccinimidyl 4-(p-maleimidophenyl)butyrate), TMEA (Tris-(2-Maleimidoethyl)amine), TSAT (Tris-(succinimidyl aminotriacetate)).

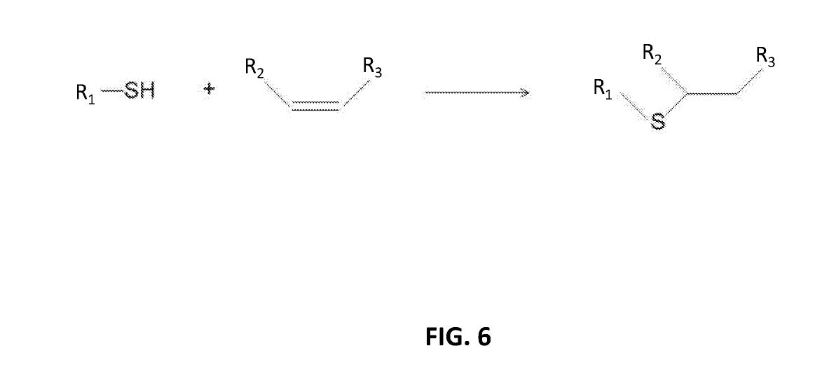

In some embodiments, CCD-conjugates or XTEN-conjugates using cross-linking reagents introduce non-natural spacer arms. However, in cases where a native peptide bond is preferred, the invention provides that a reaction can be carried out using zero-length cross-linkers that act via activation of a carboxylate group. In the embodiments thereof, in order to achieve reaction selectivity, the first polypeptide has to contain only a free C-terminal carboxyl group while all lysine, glutamic acid and aspartic acid side chains are protected and the second peptide/protein N-terminal α-amine has to be the only available unprotected amino group (requiring that any lysines, asparagines or glutamines be protected). In such cases, use of XTEN AG family sequences of Table 10 that are without glutamic acid as the first polypeptide in the XTEN-conjugate or XTEN-cross-linker is preferred. Accordingly, in one embodiment, the invention provides XTEN-cross-linker and XTEN-conjugate comprising AG XTEN sequences wherein the compositions are conjugated to payloads using a zero-length cross-linkers. Exemplary zero-length cross-linkers utilized in the embodiment include but are not limited to DCC (N,N-Dicyclohexylcarbodiimide) and EDC (1-Ethyl-3-(3-dimethylaminopropyl) carbodiimide hydrochloride) wherein the cross-linikers are used to directly conjugate carboxyl functional groups of one molecule (such as a payload) to the primary amine of another molecule, such as a payload with that functional group (see FIG. 6). Sulfo-NHS (N-hydroxysulfosuccinimide) and NHS (N-hydroxysuccinimide) are used as catalysts for conjugation, increasing reaction efficiency (Grabarek Z, Gergely J. Zero-length crosslinking procedure with the use of active esters. (1990) Anal. Biochem. 185(1), 131-135). EDC reacts with carboxylic acid group and activates the carboxyl group to form an active O-acylisourea intermediate, allowing it to be coupled to the amino group in the reaction mixture. The O-acylisourea intermediate is unstable in aqueous solutions, making it ineffective in two-step conjugation procedures without increasing the stability of the intermediate using N-hydroxysuccinimide This intermediate reacts with a primary amine to form an amide derivative. The crosslinking reaction is usually performed between pH 4.5 to 5 and requires only a few minutes for many applications. However, the yield of the reaction is similar at pH from 4.5 to 7.5. The hydrolysis of EDC is a competing reaction during coupling and is dependent on temperature, pH and buffer composition. 4-Morpholinoethanesulfonic acid (MES) is an effective carbodiimide reaction buffer. Phosphate buffers reduce the reaction efficiency of the EDC, but increasing the amount of EDC can compensate for the reduced efficiency. Tris, glycine and acetate buffers may not be used as conjugation buffers.

The disclosure also provides compositions in which three components of the targeted conjugate compositions, such as portrayed in formula VIII, below, or FIG. 34C, in which two XTENs are linked by trivalent cross-linkers to the fusion protein, resulting in trimeric conjugates. The invention also contemplates configurations wherein three molecules of a fusion protein of formula I-VII are linked by the cysteine-engineered XTEN components of the fusion proteins using trimeric cross-linkers. Trimeric cross-linkers can be created based on synthetic peptides Ac-Cys-Ser-Pro-Cys-Ser-Pro-Cys-NH₂ (SEQ ID NO: 605) or Ac-Lys-Ser-Pro-Lys-Ser-Pro-Lys-NH₂ (SEQ ID NO: 606) with various reactive side groups described in Table 24, using standard conjugation techniques.

TABLE 24
Trivalent Cross-linkers
Trivalent Cross-linker

| Trivalent Core | Group 1 | Group 2 | Group 3 |
|---|---|---|---|
| Ac- | Lys(azidoacetyl) | Lys(azidoacetyl) | Lys(azidoacetyl) |
| KSPKSPK- | Lys(maleimidopropyonyl) | Lys(maleimidopropyonyl) | Lys(maleimidopropyonyl) |
| NH₂ (SEQ ID | Lys(bromoacetyl) | Lys(bromoacetyl) | Lys(bromoacetyl) |
| NO: 606) | Lys(iodoacetyl) | Lys(iodoacetyl) | Lys(iodoacetyl) |
| AcCSPCSPC- | Cys | Cys | Cys |
| NH₂ (SEQ ID | | | |
| NO: 605) | | | |

In other embodiments, CCD or XTEN and payloads can be conjugated using a broad group of cross-linkers, including those consisting of a spacer arm (linear or branched) and two or more reactive ends that are capable of attaching to specific functional groups (e.g., primary amines, sulfhydryls, etc.) on proteins or other molecules. Linear cross-linkers can be homobifunctional or heterobifunctional. Homobifunctional cross-linkers have two identical reactive groups which are used to cross-link proteins in one step reaction procedure. Non-limiting examples of amine-reactive homobifunctional cross-linkers are BS2G (Bis (sulfosuccinimidyl)glutarate), BS3 (Sulfo-DSS) (Bis (sulfosuccinimidyl)suberate), BS[PEG]5 (Bis (NHS)PEGS), BS(PEG)9 (Bis (NHS)PEG9), BSOCOES (Bis(2-[succinimidoxycarbonyloxy]ethyl)sulfone), DSG (Disuccinimidyl glutarate), DSP (Dithiobis(succimidylpropionate) (Lomant's Reagent), DSS (Disuccinimidyl suberate), DST (Disuccinimidyl tartarate), DTSSP (Sulfo-DSP) (3,3′-Dithiobis (sulfosuccinimidyl propionate)), EGS (Ethylene glycol bis(succinimidylsuccinate)), Sulfo-EGS (Ethylene glycol bis (sulfo-succinimidyl succinate)).

Additionally, examples of homobifunctional cross-linkers employed in the compositions and in the methods to create the CCD-conjugate and/or XTEN-conjugate and/or CCD-cross-linker and/or XTEN-cross-linker compositions are sulfhydryl-reactive agents such as BMB (1,4-Bis-Maleimidobutane), BMH (Bis-Maleimidohexane), BMDB (1,4 Bismaleimidyl-2,3-dihydroxybutane), BMOE (Bis-Maleimidoethane), BM(PEG)2 (1,8-Bis-Maleimidodiethylene-glycol), BM(PEG)3 (1,11-Bis-Maleimidotriethyleneglycol), DPDPB (1,4-Di-(3′-[2′pyridyldithio]propionamido) butane), DTME (Dithiobis-maleimidoethane).

For the creation of CCD-cross-linker and XTEN-cross-linker conjugates for subsequent conjugation to payloads, heterobifunctional cross-linkers are preferred as the sequential reactions can be controlled. As heterobifunctional cross-linkers possess two different reactive groups, their use in the compositions allows for sequential two-step conjugation. A heterobifunctional reagent is reacted with a first protein using the more labile group. In one embodiment, the conjugation of the heterobifunctional cross-linker to a reactive group in a CCD or an XTEN results in a CCD-cross-linker or an XTEN-cross-linker conjugate, respectively. After completing the reaction and removing excess unreacted cross-linker, the modified protein (such as the XTEN-cross-linker) can be added to the payload which interacts with a second reactive group of the cross-linker, resulting in a CCD-conjugate or an XTEN-conjugate. Most commonly used heterobifunctional cross-linkers contain an amine-reactive group at one end and a sulfhydryl-reactive group at the other end. Accordingly, these cross-linkers are suitable for use with cysteine- or lysine-engineered XTEN, or with the alpha-amino group of the N-terminus of the XTEN. Non-limiting examples of heterobifunctional cross-linkers are AMAS (N-(α-Maleimidoacetoxy)-succinimide ester), BMPS (N-(β-Maleimidopropyloxy)succinimide ester), EMCS (N-(ε-Maleimidocaproyloxy)succinimide ester), GMBS (N-(γ-Maleimidobutyryloxy)succinimide ester), LC-SMCC (Succinimidyl 4-(N-maleimidomethyl) cyclohexane-1-carboxy-(6-amidocaproate)), LC-SPDP (Succinimidyl 6-(3′-[2-pyridyldithio]propionamido)hexanoate), MBS (m-Maleimidobenzoyl-N-hydroxysuccinimide ester), SBAP (Succinimdyl 3-(bromoacetamido)propionate), SIA (N-succinimidyl iodoacetate), SIAB (N-Succinimidyl(4-iodoacetyl)aminobenzoate), SMPB (Succinimidyl 4-(p-maleimidophenyl) butyrate), SMCC (Succinimidyl 4-(N-maleimido-methypcyclohexane-1-carboxylate), SM[PEG]₂ (NHS-PEG2-Maliemide), SM[PEG]₄ (NHS-PEG4-Maliemide), SM(PEG)₆ (NHS-PEG6-Maleimide), SM[PEG]₈ (NHS-PEG8-Maliemide), SM[PEG]₁₂ (NHS-PEG12-Maliemide), SM(PEG)₂₄ (NHS-PEG24-Maleimide), SMPB (Succinimidyl 4-(p-maleimido-phenyl)butyrate), SMPH (Succinimidyl-6-β-maleimidopropionamido)hexanoate), SMPT (4-Succinimidyloxycarbonyl-methyl-α-(2-pyridyldithio)toluene), SPDP (N-Succinimidyl 3-(2-pyridyldithio)propionate), Sulfo-EMCS (N-(ε-Maleimidocaproyloxy)sulfosuccinimide ester), Sulfo-GMBS (N-(γ-Maleimidobutryloxy)sulfosuccinimide ester), Sulfo-KMUS (N-(κ-Maleimidoundecanoyloxy)sulfosuccinimide ester), Sulfo-LC-SMPT (Sulfosuccinimidyl 6-(α-methyl-α-[2-pyridyldithio]-toluamido)hexanoate), Sulfo-LC-SPDP (Sulfosuccinimidyl 6-(3′-[2-pyridyldithio]propionamido)hexanoate), Sulfo-MBS (m-Maleimidobenzoyl-N-hydroxysulfosuccinimide ester), Sulfo-SIAB (Sulfosuccinimidyl(4-iodo-acetyl)aminobenzoate), Sulfo-SMCC (Sulfosuccinimidyl 4-(N-maleimidomethyl)cyclohexane-1-carboxylate), Sulfo-SMPB (Sulfosuccinimidyl 4-(p-maleimidophenyl)butyrate). An example of a heterobifunctional cross-linker that allows covalent conjugation of amine- and sulfhydryl-containing molecules is Sulfo-SMCC (Sulfo Sulfosuccinimidyl 4-(N-maleimidomethyl)cyclohexane-1-carboxylate). Sulfo-SMCC is a water soluble analog of SMCC that can be prepared in aqueous buffers up to 10 mM concentration. The cyclohexane ring in the spacer arm of this cross-linker decreases the rate of hydrolysis of the maleimide group compared to similar reagents not containing this ring. This feature enables CCD or XTEN that have been maleimide-activated with SMCC or Sulfo-SMCC to be lyophilized and stored for later conjugation to a sulfhydryl-containing molecule. Thus, in one embodiment, the invention provides an XTEN-cross-linker having an XTEN having at least about 80% sequence identity, or at least about 90%, or about 91%, or about 92%, or about 93%, or about 94%, or about 95%, or about 96%, or about 97%, or about 98%, or about 99% sequence identity, or is identical to a sequence or a fragment of a sequence selected from of Table 11, when optimally aligned, wherein XTEN-cross-linker has one or more cross-linkers of sulfo-SMCC linked to the α-amino group of the XTEN or the ε-amine of a lysine-engineered XTEN. In another embodiment, the invention provides an XTEN-cross-linker having an XTEN having at least about 80% sequence identity, or at least about 90%, or about 91%, or about 92%, or about 93%, or about 94%, or about 95%, or about 96%, or about 97%, or about 98%, or about 99% sequence identity, or is identical to a sequence or a fragment of a sequence selected from of Table 10, when optimally aligned, wherein the XTEN-cross-linker has one sulfo-SMCC linked to the amino group of the N-terminus of the XTEN. The foregoing described heterobifunctional cross-linkers conjugate two molecules via a single amine and a single cysteine. A special type of cross-linker was developed for site-specific conjugation to disulfide bridges in proteins (Balan S. et al. Site-specific PEGylation of protein disulfide bonds using a three-carbon bridge. (2007) Bioconjugate Chem. 18, 61-76; Brocchini S. et al. Disulfide bridge based PEGylation of proteins. (2008) Advanced Drug Delivery Reviews 60, 3-12). First, the linker is synthesized as an amine-specific 4[2,2-bis[p-tolylsulfonyl)methyl]acetyl) benzoic acid-NHS ester. This molecule can be covalently attached to the amino group of a CCD or XTEN yielding-Bis(sulfone) or XTEN-Bis(sulfone), respectively. Incubation of the latter molecule in 50 mM sodium phosphate buffer, pH 7.8, will result in elimination of toluene sulfinic acid to generate XTEN-α,β-unsaturated β′-monosulfone. The resulting molecule will react with a disulfide bridge-containing payload protein in a site-specific manner. In a first step the disulfide bridge is converted into two thiols by reduction. In a second step, the CCD-monosulfone or XTEN-monosulfone bis-alkylates two cysteines resulting in a chemically-stable three-carbon bridge. The same α,β-unsaturated β′-monosulfone can be used not only for conjugation to two thiol groups derived from a disulfide bridge but also for conjugation to polyhistidine tags (Cong Y. et al. Site-specific PEGylation at histidine tags. (2012) Bioconjugate Chem. 23, 248-263).

Conjugation using cross-linker compositions with the sulfo-SMCC is usually performed in a two-step process. In one embodiment, the amine-containing protein is prepared in conjugation buffer of, e.g., phosphate-buffered saline (PBS=100 mM sodium phosphate, 150 mM sodium chloride, pH 7.2) or a comparable amine- and sulfhydryl-free buffer at pH 6.5-7.5. The addition of EDTA to 1-5 mM helps to chelate divalent metals, thereby reducing disulfide formation in the sulfhydryl-containing protein. The concentration of the amine-containing protein determines the cross-linker molar excess to be used. In general, in protein samples of <1 mg/ml utilize an 40-80-fold molar excess, protein samples of 1-4 mg/ml utilize a 20-fold molar excess, and protein samples of 5-10 mg/ml utilize a 5- to 10-fold molar excess of the cross-linker. The reaction mixture (amine-containing protein and cross-linker) is incubated for 30 minutes at room temperature or 2 hours at 4° C. and then the excess cross-linker is removed using a desalting column equilibrated with conjugation buffer. In the case of preparing a CCD-cross-linker or XTEN-cross-linker, the composition would be held at that point. In embodiments wherein the CCD-cross-linker or XTEN-cross-linker is conjugated to a payload, the sulfhydryl-containing payload and the cross-linker conjugate are mixed in a molar ratio corresponding to that desired for the final conjugate (taking into account the number of expected cross-linkers conjugated to one or more amino groups per molecule of the CCD or XTEN) and consistent with the single sulfhydryl group that exists on the payload. The reaction mixture is incubated at room temperature for 30 minutes or 2 hours at 4° C. Conjugation efficiency can be estimated by SDS-PAGE followed by protein staining or by appropriate analytical chromatography technique such as reverse phase HPLC or cation/anion exchange chromatography.

In one embodiment, the invention provides conjugate compositions created using cross-linkers that are multivalent, resulting in compositions that have 2, 3, 4, 5, 6 or more XTEN using the synthetic peptides of composition Ac-(Lys*-Ser-Pro)ₙ-Lys*-NH₂ (SEQ ID NO: 607) (where n=1, 2, 3, 4, 5, etc., and Lys* is Lysine with ε-amino group modified to azide, maleimide, iodoacetyl, bromoacetyl, etc.). In another embodiment, the invention provides conjugate compositions created using cross-linkers that are multivalent, resulting in compositions that have 2, 3, 4, 5, 6 or more XTEN linked to 1, 2, 3, 4, 5, 6 or more different payloads. Non-limiting examples of multivalent trifunctional cross-linkers are “Y-shaped” sulfhydryl-reactive TMEA (Tris-(2-Maleimidoethyl)amine) and amine-reactive TSAT (Tris-(succimimidyl aminotricetate). Any combination of reactive moieties can be designed using a scaffold polymer, either linear or branched, for multivalent compositions. Not to be bound by a particular theory, a conjugate composition having three XTEN linked by a trifunctional linker (with payloads linked, in turn to CCD via incorporated cysteine residues) can utilize proportionally shorter XTEN for each “arm” of the construct compared to a monovalent XTEN composition wherein the same number of payloads are linked to the incorporated cysteine amino residues of each CCD, and the resulting trimeric targeted-conjugate composition will have a comparable apparent molecular weight and hydrodynamic radius as the monomeric XTEN-conjugate composition, yet will have lower viscosity, aiding administration of the composition to the subject through small-bore needles, and will provide equal or better potency from the payloads due to reduced steric hindrance and increased flexibility of the composition compared to the monomeric composition having an equivalent number of XTEN amino acids.

Cross-linkers can be classified as either “homobifunctional” or “heterobifunctional” wherein homobifunctional cross-linkers have two or more identical reactive groups and are used in one-step reaction procedures to randomly link or polymerize molecules containing like functional groups, and heterobifunctional cross-linkers possess different reactive groups that allow for either single-step conjugation of molecules that have the respective target functional groups or allow for sequential (two-step) conjugations that minimize undesirable polymerization or self-conjugation. In a preferred embodiment, where CCD-cross-linkers or XTEN-cross-linkers are prepared and isolated as compositions for subsequent reaction, the CCD-cross-linker or XTEN-cross-linker is linked to a heterbifunctional cross-linker and has at least one reactive group available for subsequent reaction.

In one embodiment, the invention provides conjugate compositions that are conjugated utilizing cleavable cross-linkers with disulfide bonds. Typically, the cleavage is effected by disulfide bond reducing agents such as β-mercaptoethanol, DTT, TCEP, however it is specifically contemplated that such compositions would be cleavable endogenously in a slow-release fashion by conditions with endogenous reducing agents (such as cysteine and glutathione). The following are non-limiting examples of such cross-linkers: DPDPB (1,4-Di-(3′-[2′pyridyldithio]propionamido) butane), DSP (Dithiobis(succimidylpropionate) (Lomant's Reagent), DTME (Dithiobis-maleimidoethane), DTSSP (Sulfo-DSP) (3,3′-Dithiobis (sulfosuccinimidylpropionate)), LC-SPDP (Succinimidyl 6-(3′-[2-pyridyldithio]propionamido)hexanoate), PDPH (3-(2-Pyridyldithio)propionylhydrazide), SMPT (4-Succinimidyloxycarbonyl-methyl-α-(2-pyridyldithio)toluene), SPDP (N-Succinimidyl 3-(2-pyridyldithio)propionate), Sulfo-LC-SMPT (Sulfosuccinimidyl 6-(α-methyl-α-[2-pyridyldithio]-toluamido)hexanoate), Sulfo-LC-SPDP (Sulfosuccinimidyl 6-(3′-[2-pyridyldithio]propionamido)hexanoate). In another embodiment, XTEN-conjugates comprising BSOCOES (Bis(2-[succinimidoxycarbonyloxy]ethyl)sulfone) can be cleaved under alkaline conditions. In another embodiment, XTEN-conjugates comprising DST (Disuccinimidyl tartarate) and BMDB (1,4 Bismaleimidyl-2,3-dihydroxybutane) can be cleaved by periodate oxidation. EGS (Ethylene glycol bis(succinimidylsuccinate)) and Sulfo-EGS (Ethylene glycol bis (sulfo-succinimidyl succinate)) are cleaved by hydroxylamine but would be expected to be cleaved endogenously such that the active payload would be released from the conjugate.

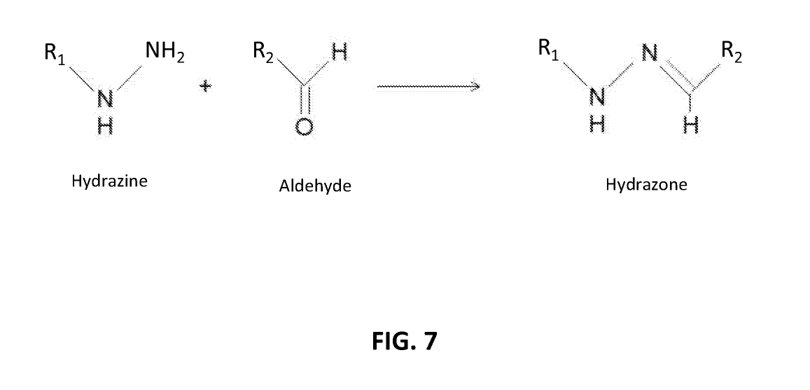

In general, the conjugation reagents described above assume that a cross-linker is reactive with the otherwise stable and inert groups such as amines, sulfhydryls and carboxyls. In other embodiments, the invention provides a different approach of conjugation based on separate modifications of the CCD, XTEN and payload with two functional groups which are stable and inactive toward biopolymers in general yet highly reactive toward each other. Several orthogonal reactions have been grouped under the concept of click chemistry, which provides XTEN-azide/alkyne reactants that have good stability properties and are therefore particularly suited as reagents for subsequent conjugation with payloads in a separate reaction (Kolb H. C., Finn M. G., Sharpless K. B. Click chemistry: diverse chemical function from a few good reactions. (2001) Angew. Chem. Int. Ed. Engl. 40(11), 2004-2021). Generally, click chemistry is used as a reaction concept which embraces reactions involving (1) alkyne-azide; (2) “ene”-thiol, and (3) aldehyde-hydrazide, and the invention contemplates use of all three. One example is the Huisgen 1,3-dipolar cycloaddition of alkynes to azides to form 1,4-disubsituted-1,2,3-triazoles, shown in FIG. 7. Azide and alkyne moieties can be introduced into peptide/protein or drug payloads or into XTEN by chemical modification of N-terminal α-amino groups, ε-amino groups of lysine, and sulfhydryl groups of cysteine. Table 25 provides non-limiting examples of click chemistry reactants contemplated for use in the making of the conjugate compositions, wherein one component of the intended conjugate (CCD, XTEN or a payload) is reacted with a reactant 1 of the Table and the second component (CCD, XTEN, or a payload) is reacted with a azide reactant 2 of the Table. For example, one molecule is modified with an alkyne moiety using an amine-reactive alkyne, such as 3-propargyloxypropanoic acid, NHS ester, acetylene-PEG4-NHS ester, dibenzylcyclooctyne (DBCO)-NHS ester, DBCO-PEG4-NHS ester, cyclooctyne (COT)-PEG2-NHS ester, COT-PEG3-NHS ester, COT-PEG4-NHS ester, COT-PEG2-pentafluorophenyl (PFP) ester, COT-PEG3-PFP ester, COT-PEG4-PFP ester, BCOT-PEG2-NHS ester, BCOT-PEG3-NHS ester, BCOT-PEG4-NHS ester, BCOT-PEG2-PFP ester, BCOT-PEG3-PFP ester, BCOT-PEG4-PFP ester. Alternatively, the molecule is modified with a sulfhydryl-reactive alkyne such as acetylene-PEG4-Maleimide, DBCO-Maleimide, or DBCO-PEG4-Maleimide. The second molecule is modified with azide-PEG2-NHS ester, azide-PEG3-NHS ester, azide-PEG4-NHS ester, azide-PEG2-PFP ester, azide-PEG3-PFP ester, azide-PEG4-PFP ester or azide-PEG4-Maleimide. The azide and alkyne moieties can be used interchangeably; they are biologically unique, stable and inert towards biological molecules and aqueous environments. When mixed, the azide and alkyne reactants form an irreversible covalent bond without any side reactions (Moses J. E. and Moorhouse A. D. The growing applications of click chemistry. (2007) Chem. Soc. Rev. 36, 1249-1262; Breinbauer R. and Kohn M. Azide-alkyne coupling: a powerful reaction for bioconjugate chemistry. (2003) ChemBioChem 4(11), 1147-1149; Rostovtsev V. V., Green L. G., Fokin V. V., Sharpless K. B. A stepwise Huisgen cycloaddition process: copper(I)-catalyzed regioselective “ligation” of azides and terminal alkynes.(2002) Angew Chem Int Ed Engl. 41(14), 2596-2599). In one embodiment, the invention provides a conjugate comprising a first XTEN conjugated to a second XTEN wherein the first XTEN is linked to a alkyne reactant 1 from Table 25, the second XTEN is linked to a azide reactant 2 from Table 25, and then the first XTEN and the second XTEN are linked under conditions effective to react the alkyne reactant 1 and the azide reactant 2, resulting in the XTEN-XTEN conjugate. In another embodiment, the invention provides a conjugate comprising a first CCD conjugated to a payload wherein the CCD is linked to a alkyne reactant 1 from Table 25, the payload is linked to a azide reactant 2 from Table 25, and then the CCD and the payload are linked under conditions effective to react the alkyne reactant 1 and the azide reactant 2, resulting in the CCD-payload-conjugate. In another embodiment, the invention provides a conjugate comprising a first CCD conjugated to a payload wherein the CCD is linked to a azide reactant 2 from Table 25, the payload is linked to a alkyne reactant 1 from Table 25, and then the CCD and the payload are linked under conditions effective to react the alkyne reactant 1 and the azide reactant 2, resulting in the CCD-payload conjugate. In the foregoing embodiments, the conditions to effect the reactions are those described herein or are reaction conditions known in the art for the conjugation of such reactants. The invention also contemplates the various combinations of the foregoing conjugates; e.g., a CCD-payload conjugate in which the components are linked by click chemistry reactants and in which one CCD further comprises one or more molecules of a payload conjugated to the CCD using click chemistry, an XTEN-CCD conjugate in which the components are linked by click chemistry reactants in which one CCD further comprises one or more molecules of a first payload conjugated to the CCD using click chemistry and the XTEN further comprises one or more molecules of a second payload conjugated to the XTEN using click chemistry. Additional variations on these combinations will be readily apparent to those of ordinary skill in the art.

TABLE 25
Alkyne and Azide Click-chemistry Reactants

| | Attached | | Attached |
|---|---|---|---|
| Alkyne Reactant 1 | to: | Azide Reactant 2 | to: |
| 3-propargyloxypropanoic | Amine | 3-azide-propionic acid, | Amine |
| acid, NHS ester* | | NHS ester* | |
| acetylene-(oxyethyl)ₙ- | Amine | 6-azide-hexanoic acid, | Amine |
| NHS ester*, where n is | | NHS ester* | |
| 1-10 | | | |
| dibenzylcyclooctyne | Amine | 3-azide-propionic acid, | Amine |
| (DBCO)-NHS ester* | | PFP ester | |
| DBCO-(oxyethyl)ₙ-NHS | Amine | 6-azide-hexanoic acid, | Amine |
| ester*, where n is 1-10 | | PFP ester | |
| cyclooctyne (COT)-NHS | Amine | azide-(oxyethyl)ₙNHS | Amine |
| ester* | | ester*, where n is 1-10 | |
| COT-(oxyethyl)ₙ-NHS | Amine | azide-(oxyethyl)ₙ-PFP | Amine |
| ester*, where n is 1-10 | | ester, where n is 1-10 | |
| COT-(oxyethyl)ₙ- | Amine | 1-azido-3,6,9,12- | Amine |
| pentafluorophenyl (PFP) | | tetraoxapentadecan-15- | |
| ester, where n is 1-10 | | oic acid N-hydroxy- | |
| | | succinimide ester | |
| BCOT-NHS ester* | Amine | azide-(oxyethyl)ₙ- | Thiol |
| | | maleimide, where n is | |
| | | 1-10 | |
| BCOT-(oxyethyl)ₙ-NHS | Amine | | |
| ester*, where n is 1-10 | | | |
| BCOT-(oxyethyl)ₙ- | Amine | | |
| pentafluorophenyl | | | |
| (PFP) ester, where | | | |
| n is 1-10 | | | |
| 6-(11,12-didehydro- | Amine | | |
| dibenzo[b,f]azocin- | | | |
| 5(6H)-yl)-6-oxohexanoic | | | |
| acid N-hydroxysulfo- | | | |
| succinimide ester | | | |
| acetylene-(oxyethyl)ₙ- | Thiol | | |
| maleimide, where n is | | | |
| 1-10 | | | |
| DBCO-maleimide | Thiol | | |
| COT-maleimide | Thiol | | |
| BCOT-maleimide | Thiol | | |

*could be either NHS ester or sulfo-NHS ester

In some embodiments, the XTEN-conjugates and the CCD-conjugates are conjugated using thio-ene based click chemistry that proceeds by free radical reaction, termed thiol-ene reaction, or anionic reaction, termed thiol Michael addition (Hoyle C. E. and Bowman C. N. Thiol-ene click chemistry. (2010) Angew. Chem. Int. Ed. 49, 1540-1573). It particular, is believed that thiol Michael addition is better suited for targeted conjugate compositions wherein the payload is a protein (Pounder R. J. et. al. Metal free thiol-maleimide ‘Click’ reaction as a mild functionalisation strategy for degradable polymers. (2008) Chem Commun (Camb). 41, 5158-5160). As at least one molecule needs to contain a free thiol group, a CCD can be utilized if the payload does not contain cysteine. Alternatively, the thiol group can be introduced by chemical modification of N-terminal α-amino group or the lysine ε-amino groups of either the XTEN, the CCD, or the payload peptide/protein using thiolating reagents such as 2-iminothiolane (Traut's reagent), SATA (N-succinimidyl 5-acetylthioacetate), SATP (N-succinimidyl S-acetylthiopropionate), SAT-PEO₄-Ac (N-Succinimidyl S-acetyl(thiotetraethylene glycol)), SPDP (N-Succinimidyl 3-(2-pyridyldithio)propionate), LC-SPDP (Succinimidyl 6-(3′-[2-pyridyldithio]propionamido)hexanoate). Such methods are known in the art (Carlsson J. et al. (1978) Biochem. J. 173, 723-737; Wang D. et al. (1997) Bioconjug. Chem. 8, 878-884; Traut R. R. et al. (1973) Biochemistry 12(17), 3266-3273; Duncan, R. J. S. et.al. (1983) Anal. Biochem. 132. 68-73; U.S. Pat. No. 5,708,146). The second component of thiol-Michael addition reaction requires a reagent with electron-deficient carbon-carbon double bond, such as in (meth)acrylates, maleimides, α,β-unsaturated ketones, fumarate esters, acrylonitrile, cinnamates, and crotonates. The N-maleimides are commonly used as sulfhydryl-reactive functionalities and can be introduced into the payload, the CCD, or the XTEN molecule via N-terminal α-amino group or Lys ε-amino group modification using commercially available heterobifunctional cross-linkers such as AMAS (N-(α-Maleimidoacetoxy)-succinimide ester), BMPS (N-(β-Maleimidopropyloxy)succinimide ester) and others described above. The resulting two molecules containing free thiol and maleimide moieties, respectively, form a stable covalent bond under mild conditions, resulting in a conjugate linked by maleimide.

In other embodiments, XTEN-conjugates, XTEN-XTEN conjugates and CCD-conjugates are created utilizing click chemistry based on reactions between hydrazides and aldehydes, such as described by Ganguly et al. and as shown in FIG. 7 (Ganguly T. et al. The hydrazide/hydrazone click reaction as a biomolecule labeling strategy for M(CO)3 (M=Re, 99 mTc) radiopharmaceuticals. (2011) Chem. Commun. 47, 12846-12848). For example, an CCD can be modified to have a hydrazine or hydrazide that is mixed with a payload having an aldehyde group to yield the desired CCD-payload conjugate. In one embodiment, the invention provides CCD with at least one hydrazine or hydrazide introduced in either the α-N-terminal amino group or, alternatively one or more lysine ε-amino groups are modified to provide an CCD suitable as a reagent for conjugation to a target payload as it is considered to be stable. The resulting bis-arylhydrazones formed from aromatic hydrazines and aromatic aldehydes are stable to 92° C. and a wide range of pH values from 2.0-10.0 (Solulink, Inc., Protein-Protein Conjugation Kit, Technical Manual, Catalog #S-9010-1). The leaving group in the reaction is water and no reducing agents (e.g., sodium cyanoborohydride) are required to stabilize the bond. Molecules modified with either hydrazine/hydrazide or aldehyde moieties have good stability in aqueous environments and remain active without special handling requirements. The amino group(s) of the CCD molecule are modified by NHS-ester/hydrazide, such as SANH (succinimidyl 4-hydrazinonicotinate acetone hydrazone), C6-SANH (C6-Succinimidyl 4-hydrazinonicotinate acetone hydrazone), SHTH (Succinimidyl 4-hydrazidoterephthalate hydrocholoride). In a typical reaction, a protein is prepared as 1-5 mg/ml solution in modification buffer (100 mM Phosphate, 150 mM NaCl, pH 7.4) and the modifying agent is added in a 5- to 20-fold molar excess and the reaction is carried out for 2 hrs at room temperature. Separately, the payload molecule is modified with NHS-ester/aldehyde SFB (succinimidyl 4-formylbenzoate) or C6-SFB (C6-Succinimidyl 4-formylbenzoate) under similar conditions. Both modified molecules are then desalted into conjugation buffer (100 mM phosphate, 150 mM NaCl, pH 6.0). The resulting components are mixed together using 1 mole equivalent of a limiting protein and 1.5-2 mole equivalents of a protein that can be used in abundance. A catalyst buffer of 100 mM aniline in 100 mM phosphate, 150 mM NaCl, pH 6.0 is added to adjust the final concentration of aniline to 10 mM and the reaction is carried out for 2 hrs at room temperature.

In another embodiment, the CCD-payload or XTEN-payload conjugate can be produced by reaction between an aldehyde and primary amino group followed by reduction of the formed Schiff base with sodium borohydride or cyanoborohydride. As a first step in the method, a CCD or an XTEN molecule, such as XTEN with a primary α-amino group or Lys-containing XTEN with an ε-amino group, is modified by NHS-ester/aldehyde SFB (succinimidyl 4-formylbenzoate), C6-SFB (C6-succinimidyl 4-formylbenzoate) or SFPA (succinimidyl 4-formylphenoxyacetate) using typical amine-NHS chemistry in an amine-free coupling buffer such as 0.1M sodium phosphate, 0.15M NaCl, pH 7.2. The resulting modified aldehyde-molecule can either be held at this point as an XTEN- or CCD-cross-linker composition or can be used as a reagent to create an targeted conjugate composition. To make the targeted conjugate composition, the modified aldehyde-CCD (which may also comprise a PCM and XTEN as a fusion protein) is mixed with a payload with a reactive amino-group and a mild reducing agent such as 20-100 mM sodium cyanoborohydride. The reaction mixture is incubated up to 6 hours at room temperature or overnight at 4° C. Unreacted aldehyde groups are then blocked with 50-500 mM Tris.HCl, pH 7.4 and 20-100 mM sodium cyanoborohydride, permitting separation of the conjugated purified conjugate.

In other embodiments, the invention provides conjugates comprising peptides or protein payloads wherein the payload is conjugated via chemical ligation based on the reactivity of the peptide/protein C-terminal acyl azide of the payload. As an example, when the peptide or protein is produced using solid-phase peptide synthesis (SPPS) with hydroxymethylbenzoic acid (HMBA) resin, the final peptide can be cleaved from the resin by a variety of nucleophilic reagents to give access to peptides with diverse C-terminal functionalities. In one embodiment, the method includes hydrazinolysis of the peptidyl/protein resins to yield peptide or protein hydrazides. Nitrosation of resulting acyl hydrazides with sodium nitrite or tent-butyl nitrite in dilute hydrochloric acid then results in formation of acyl azides. The resulting carbonyl azide (or acyl azide) is an activated carboxylate group (esters) that can react with a primary amine of an XTEN or a CCD to form a stable amide bond, resulting in the conjugate. In alternative embodiments, the primary amine could be the α-amine of the XTEN or CCD N-terminus or one or more a-amine of engineered lysine residues in the XTEN sequence. In the conjugation reaction, the azide function is the leaving group. The conjugation reaction with the amine groups occurs by attack of the nucleophile at the electron-deficient carbonyl group (Meienhofer, J. (1979) The Peptides: Analysis, Synthesis, Biology. Vol. 1, Academic Press: N.Y.; ten Kortenaar P. B. W. et. al. Semisynthesis of horse heart cytochrome c analogues from two or three fragments. (1985) Proc. Natl. Acad. Sci. USA 82, 8279-8283)

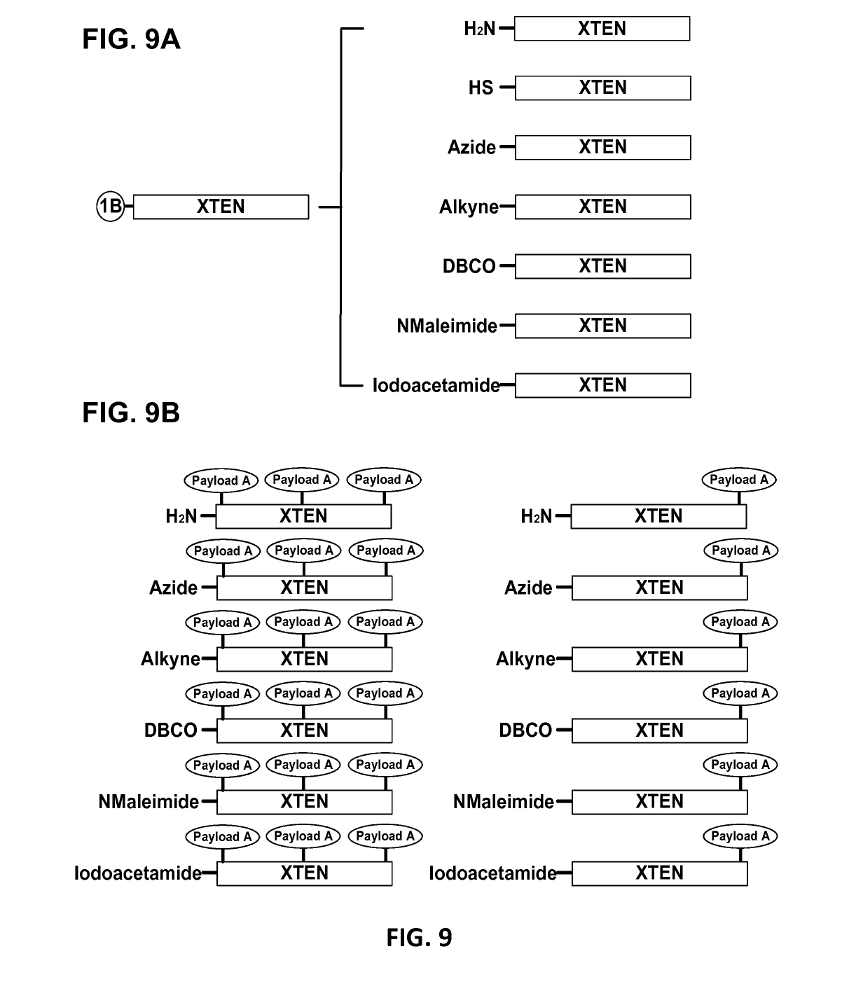

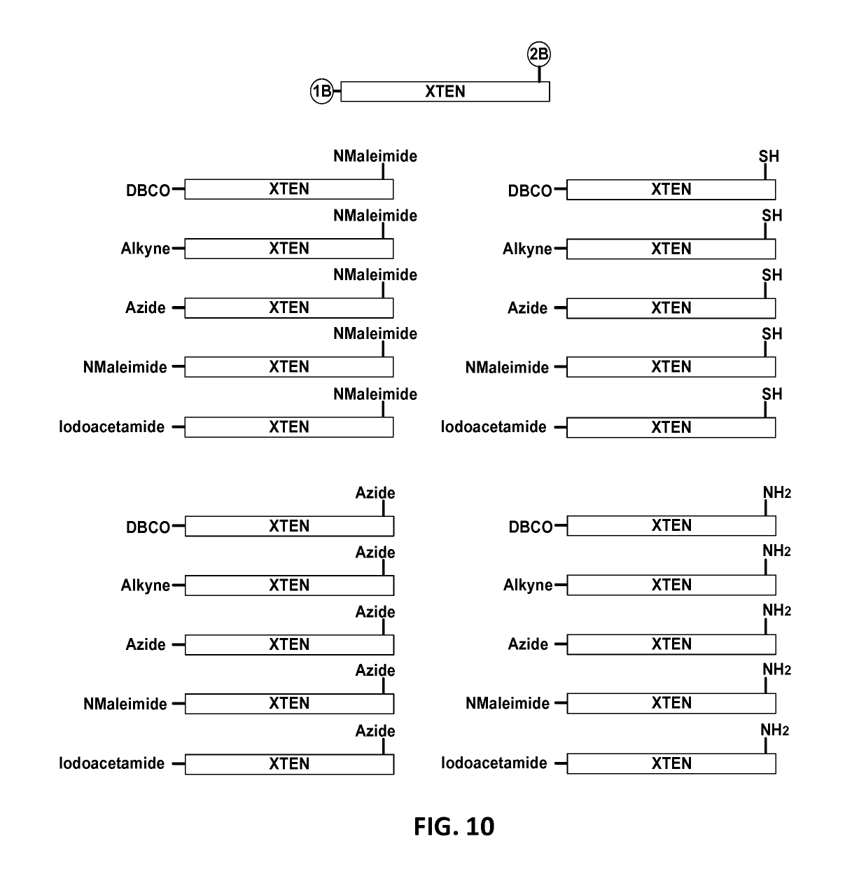

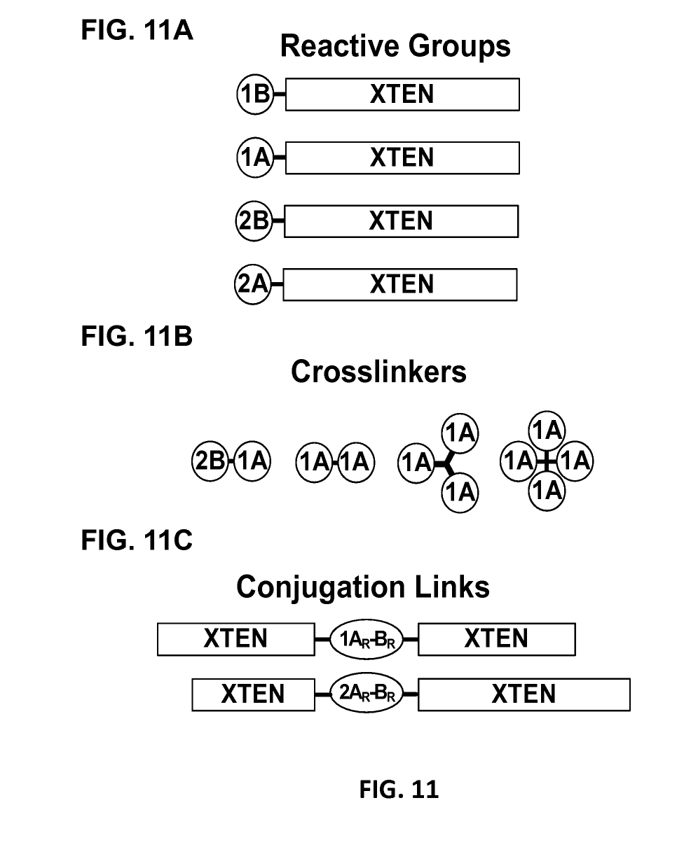

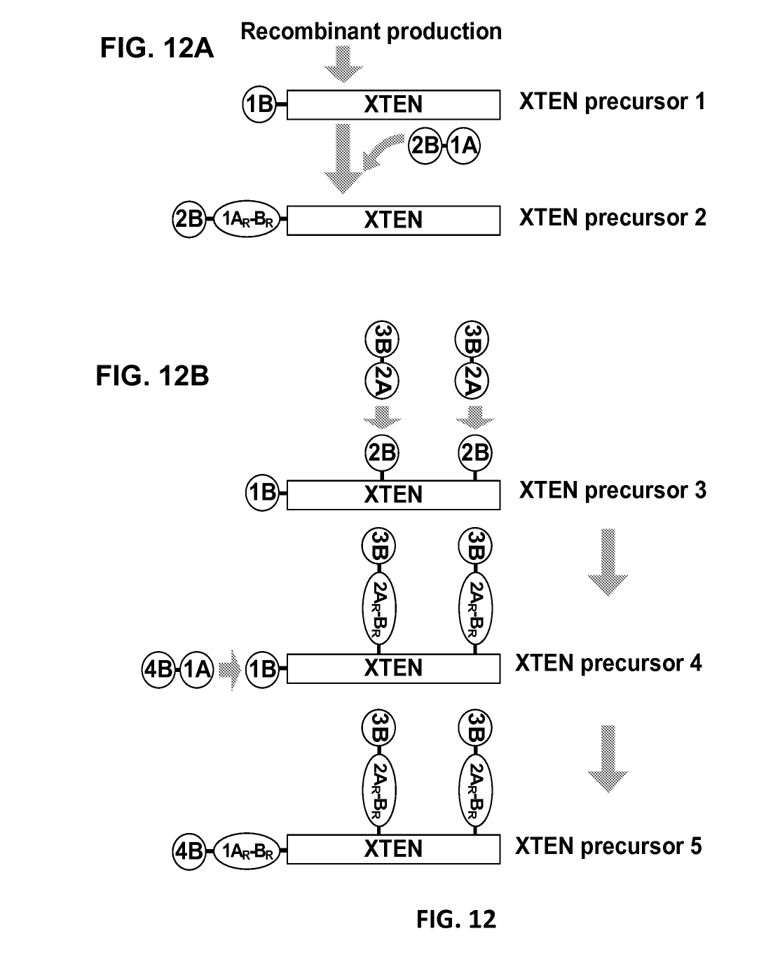

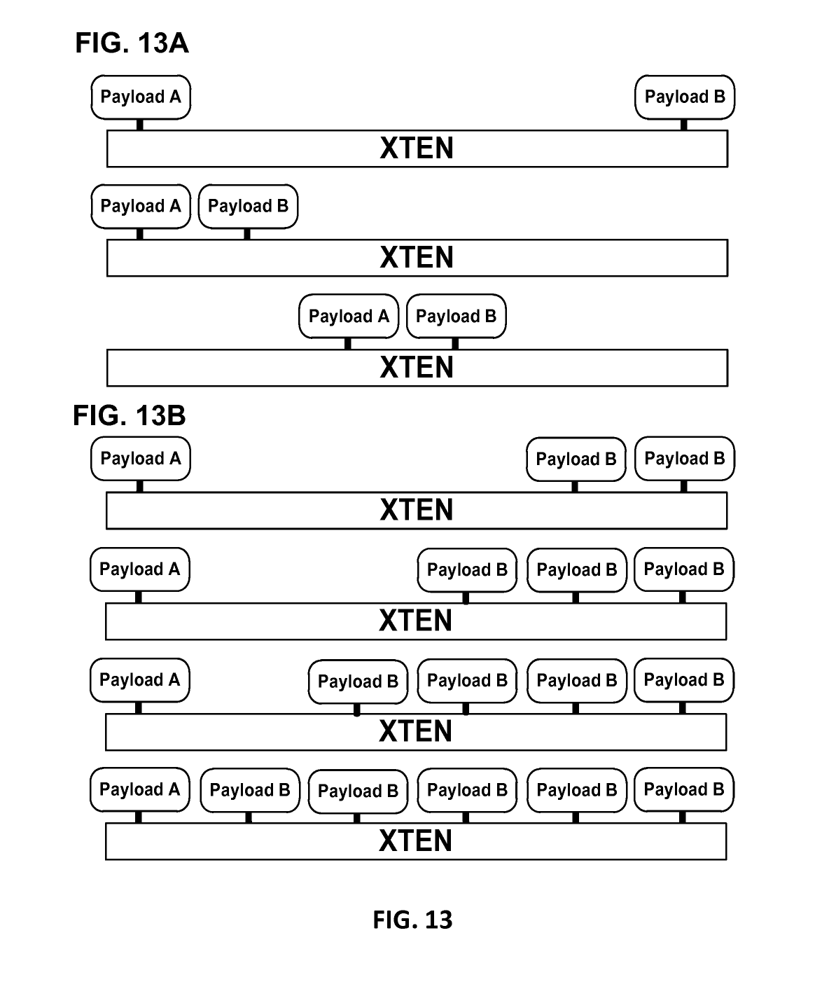

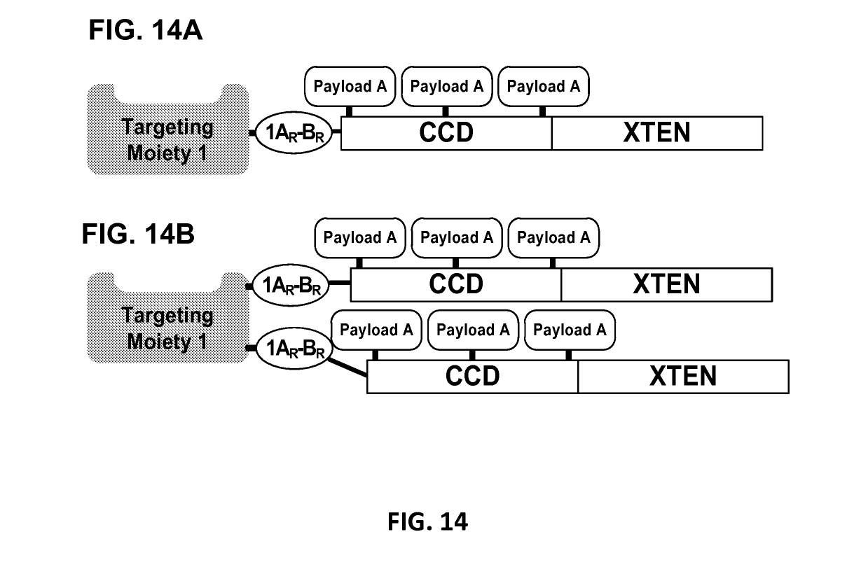

In another embodiment, the invention provides targeted conjugate compositions prepared by enzymatic ligation. Transglutaminases are enzymes that catalyze the formation of an isopeptide bond between the γ-carboxamide group of glutamine of a payload peptide or protein and the ε-amino group of a lysine in a lysine-engineered XTEN, thereby creating inter- or intramolecular cross-links between the XTEN and payload (see FIG. 9), resulting in the composition (Lorand L, Conrad S.M. Transglutaminases.(1984) Mol. Cell Biochem. 58(1-2), 9-35). Non-limiting examples of enzymes that have been successfully used for ligations are factor XIIIa (Schense J. C., Hubbell J. A. Cross-linking exogenous bifunctional peptides into fibrin gels with factor XIIIa. (1999) Bioconjug. Chem. 10(1):75-81) and tissue transglutaminase (Collier J. H., Messersmith P. B. Enzymatic modification of self-assembled peptide structures with tissue transglutaminase. (2003) Bioconjug. Chem. 14(4), 748-755; Davis N. E., Karfeld-Sulzer L. S., Ding S., Barron A. E. Synthesis and characterization of a new class of cationic protein polymers for multivalent display and biomaterial applications. (2009) Biomacromolecules 10 (5), 1125-1134). The glutamine substrate sequence GQQQL (SEQ ID NO: 608) is known to have high specificity toward tissue transglutaminase (Hu B. H., Messersmith P. B. Rational design of transglutaminase substrate peptides for rapid enzymatic formation of hydrogels.(2003) J. Am. Chem. Soc. 125(47), 14298-14299). Tissue transglutaminase sequence specificity was less stringent for an acyl acceptor (lysine) than for acyl donor (glutamine) (Greenberg C. S., Birckbichler P. J., Rice R. H. Transglutaminases: multifunctional cross-linking enzymes that stabilize tissues. (1991) FASEB J. 1991, 5, 3071-3077).

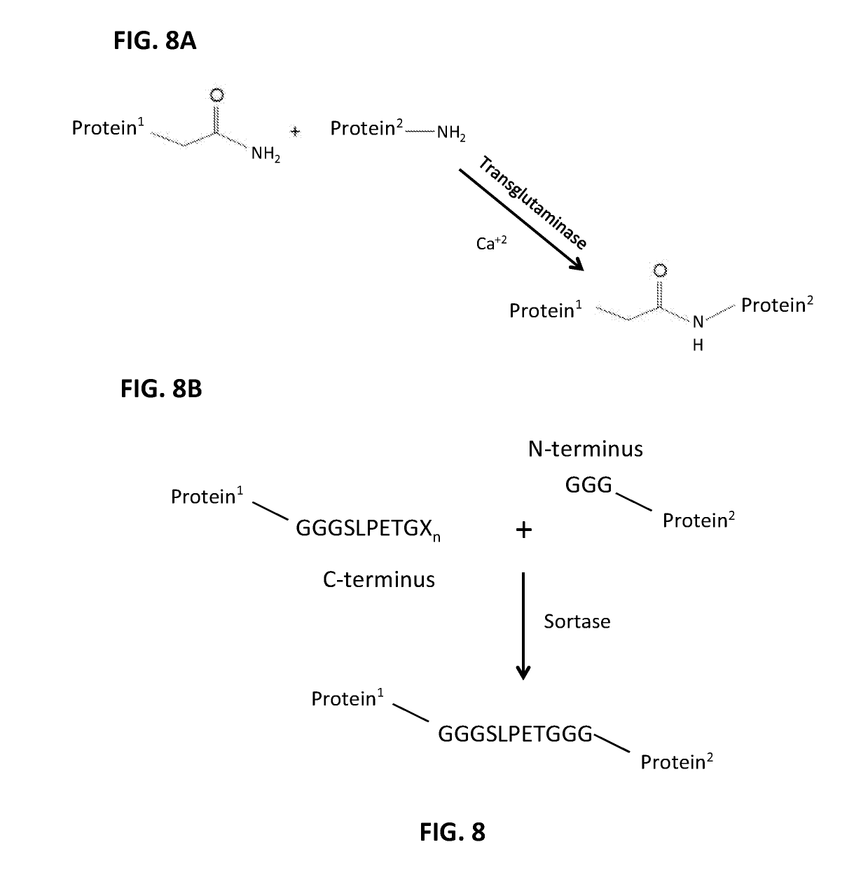

In an alternative embodiment of an enzymatically-created targeted conjugate composition, the sortase A transpeptidase enzyme from Staphylococcus aureus is used to catalyze the cleavage of a short 5-amino acid recognition sequence LPXTG (SEQ ID NO: 3) between the threonine and glycine residues of Protein1, and subsequently transfers the acyl-fragment to an N-terminal oligoglycine nucleophile of Protein1 (see FIG. 8). By functionalizing the Protein2 to contain the oligoglycine, it is possible to enzymatically conjugate the two proteins in a site-specific fashion to result in the desired targeted conjugate composition composition. The (poly)peptide bearing the sortase recognition site (LPXTG) (SEQ ID NO: 3) can be readily made using standard molecular biology cloning protocols. It is convenient to introduce glutamic acid in the X position of the recognition site, as this residue is commonly found in natural substrates of sortase A (Boekhorst J., de Been M. W., Kleerebezem M., Siezen R. J. Genome-wide detection and analysis of cell wall-bound proteins with LPxTG-like sorting motifs (SEQ ID NO: 3). (2005) J. Bacteriol. 187, 4928-4934). A high level of transacylation can be achieved by placing the sortase cleavage site both at the C-terminus of the substrate (Popp M. W., Antos J. M., Grotenbreg G. M., Spooner E., Ploegh H. L. Sortagging: A versatile method for protein labeling. (2007) Nat. Chem. Biol. 311,707-708) and in flexible loops (Popp M. W., Artavanis-Tsakonas K., Ploegh H. L. Substrate filtering by the active-site crossover loop in UCHL3 revealed by sortagging and gain-of-function mutations. (2009) J. Biol. Chem. 284(6), 3593-3602). For proteins labeled at the C-terminus, it is important that the glycine in the minimal LPETG tag (SEQ ID NO: 609) is not placed at the C-terminus; it must be in a peptide linkage with at least one further C-terminal amino acid. In addition, better linkage is achieved by adding an extra glycine to the C-terminus of the cleavage site to yield LPETGG (SEQ ID NO: 610) (Pritz S., Wolf Y., Kraetke O., Klose J., Bienert M., Beyermann M. Synthesis of biologically active peptide nucleic acid-peptide conjugates by sortase-mediated ligation. (2007) J. Org. Chem. 72, 3909-3912; Tanaka T., Yamamoto T., Tsukiji S., Nagamune T. Site-specific protein modification on living cells catalyzed by sortase. (2008) Chembiochem 95, 802-807). Nucleophiles compatible with sortase-mediated transpeptidation have the single structural requirement of a stretch of glycine residues with a free amino terminus. Successful transpeptidation can be achieved with nucleophiles containing anywhere from one to five glycines; however, in a preferred embodiment, a maximum reaction rate is obtained when two or three glycines are present.

While the various embodiments of conjugation chemistry have been described in terms of protein-protein conjugations, it is specifically intended that in practicing the invention, the payload moiety of the targeted conjugate compositions can be a small molecule drug in those conjugation methods applicable to functional groups like amines, sulfhydryls, carboxyl that are present in the target small molecule drugs. It will be understood by one of ordinary skill in the art that one can apply even more broad chemical techniques compared to protein and peptides whose functionalities are usually limited to amino, sulfhydryl and carboxyl groups. Drug payloads can be conjugated to the XTEN through functional groups including, but not limited to, primary amino groups, aminoxy, hydrazide, hydroxyl, thiol, thiolate, succinate (SUC), succinimidyl succinate (SS), succinimidyl propionate (SPA), succinimidyl butanoate (SBA), succinimidyl carboxymethylate (SCM), benzotriazole carbonate (BTC), N-hydroxysuccinimide (NHS), p-nitrophenyl carbonate (NPC). Other suitable reactive functional groups of drug molecule payloads include acetal, aldehydes (e.g., acetaldehyde, propionaldehyde, and butyraldehyde), aldehyde hydrate, alkenyl, acrylate, methacrylate, acrylamide, active sulfone, acid halide, isocyanate, isothiocyanate, maleimide, vinylsulfone, dithiopyridine, vinylpyridine, iodoacetamide, epoxide, glyoxal, dione, mesylate, tosylate, and tresylate.

In some embodiments of the XTEN-conjugates with drugs as the payload, the drug molecules are attached to lysine- or cysteine engineered XTEN (such as the sequences of Table 11) or the CCD of Table 6 by cross-linkers having two reactive sites for binding to the drug and the XTEN or the CCD. Preferred cross-linker groups are those that are relatively stable to hydrolysis in the circulation, are biodegradable and are nontoxic when cleaved from the conjugate. In addition, the use of cross-linkers can provide the potential for conjugates with an even greater flexibility between the drug and the CCD or XTEN, or provide sufficient space between the drug and the CCD or XTEN such that the CCD or XTEN does not interfere with the binding between the pharmacophore and its binding site. In one embodiment, a cross-linker has a reactive site that has an electrophilic group that is reactive to a nucleophilic group present on a CCD or an XTEN. Preferred nucleophiles include thiol, thiolate, and primary amine. The heteroatom of the nucleophilic group of a lysine- or cysteine-engineered XTEN or the CCD comprising cysteine is reactive to an electrophilic group on a cross-linker and forms a covalent bond to the cross-linker unit, resulting in a cross-linker conjugate. Useful electrophilic groups for cross-linkers include, but are not limited to, maleimide and haloacetamide groups, and provide a convenient site for attachment to the XTEN. In another embodiment, a cross-linker has a reactive site that has a nucleophilic group that is reactive to an electrophilic group present on a drug such that a conjugation can occur between the XTEN-cross-linker or the CCD-cross-liner and the payload drug, resulting in a conjugate. Useful electrophilic groups on a drug include, but are not limited to, hydroxyl, thiol, aldehyde, alkene, alkane, azide and ketone carbonyl groups. The heteroatom of a nucleophilic group of a cross-linker can react with an electrophilic group on a drug and form a covalent bond. Useful nucleophilic groups on a cross-linker include, but are not limited to, hydrazide, oxime, amino, hydrazine, thiosemicarbazone, hydrazine carboxylate, and arylhydrazide. The electrophilic group on a drug provides a convenient site for attachment to a cross-inker.

In a particular embodiment, the conjugation of drugs to the lysine epsilon amino group of a subject lysine-engineered XTEN makes use of a reactive drug-N-hydroxylsuccinimide reactant, or esters such as drug-succinimidyl propionate, or drug-succinimidyl butanoate or other drug-succinimide conjugates. Alternatively, lysine residues of the subject lysine-engineered XTEN may be used to introduce free sulfhydryl groups through reaction with 2-iminothiolane. Alternatively, targeting substance lysines of subject lysine-engineered XTEN may may be linked to a heterobifunctional reagent having a free hydrazide or aldehyde group available for conjugation with an active drug agent. Reactive esters can conjugate at physiological pH, but less reactive derivatives typically require higher pH values. Low temperatures may also be employed if a labile protein payload is being used. Under low temperature conditions, a longer reaction time may be used for the conjugation reaction.

In another particular embodiment, the invention provides XTEN-conjugates with an amino group conjugation with lysine residues of a subject lysine-engineered XTEN wherein the conjugation is facilitated by the difference between the pKa values of the α-amino group of the N-terminal amino acid (approximately 7.6 to 8.0) and pKa of the ε-amino group of lysine (approximately 10). Conjugation of the terminal amino group often employs reactive drug-aldehydes (such as drug-propionaldehyde or drug-butylaldehyde), which are more selective for amines and thus are less likely to react with, for example, the imidazole group of histidine. In addition, amino residues are reacted with succinic or other carboxylic acid anhydrides, or with N,N′-Disuccinimidyl carbonate (DSC), N,N′-carbonyl diimidazole (CDI), or p-nitrophenyl chloroformate to yield the activated succinimidyl carbonate, imidazole carbamate or p-nitrophenyl carbonate, respectively. Derivatization with these agents has the effect of reversing the charge of the lysinyl residues. Conjugation of a drug-aldehyde to the terminal amino group of a subject XTEN typically takes place in a suitable buffer performed at a pH which allows one to take advantage of the pKa differences between the ε-amino groups of the lysine residues and that of the α-amino group of the N-terminal residue of the protein. In the method of the embodiment, the reaction for coupling uses a pH in the range of from about pH 7 up to about 8. Useful methods for conjugation of the lysine epsilon amino group have been described in U.S. Pat. No. 4,904,584 and U.S. Pat. No. 6,048,720.

The activation method and/or conjugation chemistry to be used in the creation of the targeted conjugate compositions depends on the reactive groups of the polypeptide as well as the functional groups of the drug moiety (e.g., being amino, hydroxyl, carboxyl, aldehyde, sulfhydryl, alkene, alkane, azide, etc), the functional group of the drug-cross-linker reactant, or the functional group of the XTEN-cross-linker or the CCD-cross-linker reactant. The drug conjugation may be directed towards conjugation to all available attachment groups on the engineered XTEN polypeptide or the CCD such as the specific engineered attachment groups on the incorporated cysteine residues or lysine residues. In order to control the reactants such that the conjugation is directed to the appropriate reactive site, the invention contemplates the use of protective groups during the conjugation reaction. A “protecting group” is a moiety that prevents or blocks reaction of a particular chemically reactive functional group in a molecule under certain reaction conditions. The protecting group will vary depending upon the type of chemically reactive group being protected as well as the reaction conditions to be employed, as well as the presence of additional reactive groups in the molecule. Non-limiting examples of functional groups which may be protected include carboxylic acid groups, hydroxyl groups, amino groups, thiol groups, and carbonyl groups. Representative protecting groups for carboxylic acids and hydroxyls include esters (such as a p-methoxybenzyl ester), amides and hydrazides; for amino groups, carbamates (such as tert-butoxycarbonyl) and amides; for hydroxyl groups, ethers and esters; for thiol groups, thioethers and thioesters; for carbonyl groups, acetals and ketals; and the like. Such protecting groups are well-known to those skilled in the art and are described, for example, in T. W. Greene and G. M. Wuts, Protecting Groups in Organic Synthesis, Third Edition, Wiley, New York, 1999, and references cited therein. The conjugation may be achieved in one step or in a stepwise manner (e.g., as described in WO 99/55377), such as through addition of a reaction intermediate cross-linker, using the cross-linkers disclosed herein or those known in the art to be useful for conjugation to cysteine or lysine residues of polypeptides to be linked to reactive functional groups on drug molecules.

In some embodiments of the invention, the method for conjugating a cross-linker to a cysteine-engineered XTEN or CCD may provide that the XTEN or CCD is pre-treated with a reducing agent, such as dithiothreitol (DTT) to reduce any cysteine disulfide residues to form highly nucleophilic cysteine thiol groups (—CH₂ SH). The reducing agent is subsequently removed by any conventional method, such as by desalting. The reduced XTEN or CCD thus reacts with drug-linker compounds, or cross-linker reagents, with electrophilic functional groups such as maleimide or a-halo carbonyl, according to, for example, the conjugation method of Klussman et al. (2004) Bioconjugate Chemistry 15(4), 765-773. Conjugation of a cross-linker or a drug to a cysteine residue typically takes place in a suitable buffer at pH 6-9 at temperatures varying from 4° C. to 25° C. for periods up to about 16 hours. Alternatively, the cysteine residues can be derivatized. Suitable derivatizing agents and methods are well known in the art. For example, cysteinyl residues most commonly are reacted with α-haloacetates (and corresponding amines), such as iodoacetic acid or iodoacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

In some instances, the conjugation is performed under conditions aiming at reacting as many of the available attachment groups as possible with drug or drug-linker molecules. This is achieved by means of a suitable molar excess of the drug in relation to the polypeptide. Typical molar ratios of activated drug or drug-linker molecules to polypeptide are up to about 1000-1, such as up to about 200-1 or up to about 100-1. In some cases, the ratio may be somewhat lower, however, such as up to about 50-1, 10-1 or 5-1. Equimolar ratios also may be used.

In preferred embodiments, the targeted conjugate compositions of the disclosure retain at least a portion of the pharmacologic activity compared to the corresponding payload not linked to the targeted conjugate composition. In one embodiment, the targeted conjugate composition retains at least about 1%, or at least about 5%, or at least about 10%, or at least about 20%, or at least about 30%, or at least about 40%, or at least about 50%, or at least about 60%, or at least about 70%, or at least about 80%, or at least about 90%, or at least about 95% of the pharmacologic activity of the payload not linked to the targeted conjugate composition.

In one embodiment, targeted conjugate compositions can be designed to release the payload in the body by unspecific or enzymatic hydrolysis of the linker, including disulfide bond reduction, pH-dependent release, or by exogenous or endogenous proteases, including the proteases of Table 6. Macromolecules can be taken up by the cell either through receptor-mediated endocytosis, adsorptive endocytosis or fluid phase endocytosis (Jain R.K. Transport of molecules across tumor vasculature. (1987) Cancer Metastasis Rev. 6(4), 559-593; Jain R. K. Transport of molecules, particles, and cells in solid tumors. (1999) Ann. Rev. Biomed. Eng. 1, 241-263; Mukherjee S., Ghosh R. N., Maxfield F. R. Endocytosis. (1997) Physiol. Rev. 77(3), 759-803). Upon cellular uptake of targeted conjugate composition, the payload can be released by low pH values in endosomes (pH 5.0-6.5) and lysosomes (pH 4.5-5.0), as well as by lysosomal enzymes (e.g., esterases and proteases). Example of acid-sensitive cross-linker is 6-maleimidodocaproyl hydrazone which can be coupled to thiol-bearing carriers. The hydrazone linker is rapidly cleaved at pH values <5 allowing a release of the payload in the acidic pH of endosomes and lysosomes following internalization of the conjugate (Trail P. A. et al. Effect of linker variation on the stability, potency, and efficacy of carcinoma-reactive BR64-doxorubicin immunoconjugates. (1997) Cancer Res. 57(1), 100-105; Kratz F. et al. Acute and repeat-dose toxicity studies of the (6-maleimidocaproyl)hydrazone derivative of doxorubicin (DOXO-EMCH), an albumin-binding prodrug of the anticancer agent doxorubicin. (2007) Hum. Exp. Toxicol. 26(1), 19-35). Clinically approved mAb-drug conjugate, gemtuzumab ozogamicin (Mylotarg™) is a drug-antibody conjugate containing a humanized mAb P67.6 against CD33, linked chemically to the cytotoxic antibiotic agent calicheamicin. The linker between the antibody and the drug incorporates two labile bonds: a hydrazone and a sterically hindered disulfide. It has been shown that the acid-sensitive hydrazone bond is the actual cleavage site (Jaracz S., Chen J., Kuznetsova L. V., Ojima I. Recent advances in tumor-targeting anticancer drug conjugates. (2005) Bioorg. Med. Chem. 13(17), 5043-5054).

For those targeted conjugate compositions in which the payload is linked by a disulfide bond, the payload can be released by reduction of disulfide bond within the labile linker. For example, huN901-DM1 is a tumor-activated immunotherapeutic prodrug developed by ImmunoGen, Inc. for the treatment of small cell lung cancer. The prodrug consists of humanized anti-CD56 mAb (huN901) conjugated with microtubule inhibitor maytansinoid DM1. An average of 3.5-3.9 molecules of DM1 are bound to each antibody via hindered disulfide bonds. Although the disulfide link is stable in blood, it is cleaved rapidly on entering the cell targeted by huM901, thus releasing active DM1 (Smith S. V. Technology evaluation: huN901-DM1, ImmunoGen. (2005) Curr. Opin. Mol. Ther. 7(4), 394-401). DM1 has been also coupled to Millennium Pharmaceuticals MLN-591, an anti-prostate-specific membrane antigen mAb. DM1 is linked to the antibody via a hindered disulfide bond that provides serum stability at the same time as allowing intracellular drug release on internalization (Henry M. D. et al. A prostate-specific membrane antigen-targeted monoclonal antibody-chemotherapeutic conjugate designed for the treatment of prostate cancer. (2004) Cancer Res. 64(21), 7995-8001).

Release of the payload from the targeted conjugate composition can be achieved by creating compositions using short cleavable peptides as linkers between the payload and the CCD or engineered XTEN. Example of the conjugate assessed clinically is doxorubicin-HPMA (N-(2-hydroxypropyl)methacrylamide) conjugate in which doxorubicin is linked through its amino sugar to the HPMA copolymer via a tetrapeptide spacer GlyPheLeuGly (SEQ ID NO: 611) that is cleaved by lysosomal proteases, such as cathepsin B (Vasey P. A. et al. Phase I clinical and pharmacokinetic study of PK1 [N-(2-hydroxypropyl)methacrylamide copolymer doxorubicin]: first member of a new class of chemotherapeutic agents-drug-polymer conjugates. (1999) Clin. Cancer Res. 5(1), 83-94). Other examples of carrier-drug conjugates with peptide linkers that reached clinical stage of development are macromolecular platinum complexes. Two HPMA-based drug candidates consisted of a HPMA copolymer backbone to which the complexing aminomalonate platinum complexes were bound through cathepsin B-cleavable peptide spacer GlyPheLeuGly (SEQ ID NO: 611) or tripeptide spacer GlyGlyGly (Rademaker-Lakhai J. M. et al. A Phase I and pharmacological study of the platinum polymer AP5280 given as an intravenous infusion once every 3 weeks in patients with solid tumors. (2004) Clin. Cancer Res. 10(10), 3386-3395; Sood P. et al. Synthesis and characterization of AP5346, a novel polymer-linked diaminocyclohexyl platinum chemotherapeutic agent. (2006) Bioconjugate Chem. 17(5), 1270-1279).

A highly selective method was developed to target prostate cancer via prostate-specific antigen (PSA) protease which is almost exclusively expressed in prostate tissue and prostate carcinomas. A novel albumin-binding prodrug of paclitaxel, EMC-ArgSerSerTyrTyrSerLeu-PABC-paclitaxel (SEQ ID NO: 612) (EMC: ε-maleimidocaproyl; PABC: p-aminobenzyloxycarbonyl) was synthesized. This prodrug was water soluble and was bound to endogenous and exogenous albumin. Albumin-bound form of the prodrug was cleaved by PSA releasing the paclitaxel-dipeptide Ser-Leu-PABC-paclitaxel. Due to the incorporation of a PABC self-eliminating linker, this dipeptide was rapidly degraded to liberate paclitaxel as a final cleavage product (Elsadek B. et al. Development of a novel prodrug of paclitaxel that is cleaved by prostate-specific antigen: an in vitro and in vivo evaluation study. (2010) Eur. J. Cancer 46(18), 3434-3444).

Self-immolative spacers have gained significant interest due to their utility in prodrug delivery systems. Several reports described linear self-eliminating systems or dendrimeric structures which can release all of their units through a domino-like chain fragmentation, initiated by a single cleavage event (Haba K. et al. Single-triggered trimeric prodrugs. (2005) Angew. Chem., Int. Ed. 44, 716-720; Shabat D. Self-immolative dendrimers as novel drug delivery platforms. (2006) J. Polym. Sci., Part A: Polym. Chem. 44, 1569-1578.Warnecke A., Kratz F. 2,4-Bis(hydroxymethyl)aniline as a building block for oligomers with self-eliminating and multiple release properties. (2008) J. Org. Chem. 73, 1546-1552; Sagi A. et al. Self-immolative polymers. (2008) J. Am. Chem. Soc. 130, 5434-5435). In one study, a self-immolative dendritic prodrug with four molecules of the anticancer agent camptothecin and two molecules of PEG5000 was designed and synthesized. The prodrug was effectively activated by penicillin-G-amidase under physiological conditions and free camptothecin was released to the reaction media to cause cell-growth inhibition (Gopin A. et al. Enzymatic activation of second-generation dendritic prodrugs: conjugation of self-immolative dendrimers with poly(ethylene glycol) via click chemistry. (2006) Bioconjugate Chem. 17, 1432-1440). Incorporation of a specific enzymatic substrate, cleaved by a protease that is overexpressed in tumor cells, could generate highly efficient cancer-cell-specific dendritic prodrug activation systems. Non-limiting examples of sequences that are cleavable by proteases are listed in Table 6.

In some embodiments, the invention provides targeted conjugate composition configurations, including dimeric, trimeric, tetrameric and higher order conjugates in which the payload is attached to the XTEN using a labile linker as described herein, above. In one embodiment of the foregoing, the composition further includes a targeting component to deliver the composition to a ligand or receptor on a targeted cell. In another embodiment, the invention provides conjugates in which one, two, three, or four individual conjugate compositions are conjugated with labile linkers to antibodies or antibody fragments, providing soluble compositions for use in targeted therapy of clinical indications such as, but not limited to, various treatment of tumors and other cancers wherein the antibody provides the targeting component and then, when internalized within the target cell, the labile linker permits the payload to disassociate from the composition and effect the intended activity (e.g, cytotoxicity in a tumor cell).

The unstructured characteristics and uniform composition and charge of XTEN result in properties that can be exploited for purification of targeted conjugate compositions following a conjugation reaction. Of particular utility is the capture of targeted conjugate compositions by ion exchange, which allows the removal of un-reacted payload and payload derivatives. Of particular utility is the capture of conjugates by hydrophobic interaction chromatography (HIC). Due to their hydrophilic nature, most XTEN polypeptides show low binding to HIC resins, which facilitates the capture of targeted conjugate compositions due to hydrophobic interactions between the payload and the column material, and their separation from un-conjugated composition that failed to conjugate to the payload during the conjugation process. The high purity of XTEN and targeted conjugate compositions offers a significant benefit compared to most chemical or natural polymers, particularly pegylated payloads. Most chemical and natural polymers are produced by random-or semi-random polymerization, which results in the generation of many homologs. Such polymers can be fractionated by various methods to increase fraction of the target entity in the product. However, even after enrichment most preparations of natural polymers and their payload conjugates contain less than 10% target entity. Examples of PEG conjugates with G-CSF have been described in [Bagal, D., et al. (2008) Anal Chem, 80: 2408-18]. This publication shows that even a PEG conjugate that is approved for therapeutic use contains more than 100 homologs that occur with a concentration of at least 10% of the target entity.

The complexity of random polymers, such as PEG, is a significant impediment for the monitoring and quality control during conjugation and purification. In contrast, XTEN purified by the methods described herein have high levels of purity and uniformity. In addition, the conjugates created as described herein routinely contain greater than about 80%, 90%, 91%, 92%, 93%, 94%, 95%, 96%, 97%, 98%, or 99% of the intended target and in the intended configuration, resulting in easy to interpret mass spectra and chromatograms.

5. Targeted Conjugate Composition Configurations

It is an object of the invention to provide different configurations of the targeted conjugate compositions or configurations with multiple numbers of a given type of component in order to confer tailored properties to the resulting compositions.

In one aspect, the invention provides monomeric targeted conjugate compositions having single copies of a targeting moiety, a CCD, an XTEN, a payload (e.g. a drug or biologically active protein) conjugated to each cysteine moiety in the CCD, and optionally a PCM.

In one embodiment, the targeted conjugate composition is configured according to the structure of formula I:

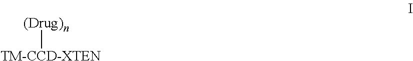

wherein: i) the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; ii) the CCD is selected from the group consisting of the CCD of Table 6; iii) the XTEN has at least 90%, or 91%, or 92%, or 93%, or 94%, or 95%, or 96%, or 97%, or 98%, or 99% identity or is identical to a sequence set forth in Table 10; and iv) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula II:

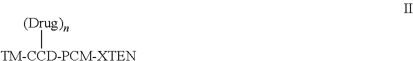

wherein: i) the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; ii) the CCD is selected from the group consisting of the CCD of Table 6; iii) the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; and v) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula III:

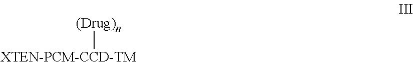

wherein: i) the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; ii) the CCD is selected from the group consisting of the CCD of Table 6; iii) the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; and v) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula IV:

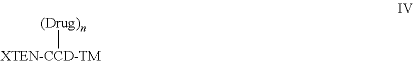

wherein: i) the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; ii) the CCD is selected from the group consisting of the CCD of Table 8; iii) the XTEN has at least 90%, or 91%, or 92%, or 93%, or 94%, or 95%, or 96%, or 97%, or 98%, or 99% identity or is identical to a sequence set forth in Table 10; and iv) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula V:

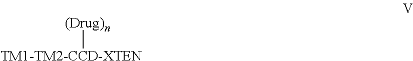

wherein: i) the TM1 and TM2 are different scFv, each comprising a VL and a VH sequence, wherein each VL and VH is derived from a first and a second antibody of Table 19 or wherein each has at least 90%, or 91%, or 92%, or 93%, or 94%, or 95%, or 96%, or 97%, or 98%, or 99% identity or is identical to a VL and a VH from a first and a second antibody selected from the group consisting of the VL and VH sequences set forth in Table 19, and further comprises a linker sequence selected from the group consisting of the sequences set forth in Table 20 wherein the linker is recombinantly fused between the VL and the VH and the TM1 and TM2 are recombinantly fused together by a short linker of hydrophilic amino acids selected from the group consisting of the sequences SGGGGS (SEQ ID NO: 1), GGGGS (SEQ ID NO: 2), GGS, and GSP; ii) the CCD is selected from the group consisting of the CCD of Table 6; iii) the XTEN has at least 90%, or 91%, or 92%, or 93%, or 94%, or 95%, or 96%, or 97%, or 98%, or 99% identity or is identical to a sequence set forth in Table 10; and iv) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula VI:

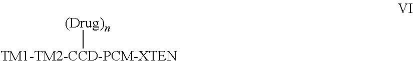

wherein: i) the TM1 and TM2 are different scFv, each comprising a VL and a VH sequence, wherein each VL and VH is derived from a first and a second antibody of Table 19 or wherein each has at least 90%, or 91%, or 92%, or 93%, or 94%, or 95%, or 96%, or 97%, or 98%, or 99% identity or is identical to a VL and a VH from a first and a second antibody selected from the group consisting of the VL and VH sequences set forth in Table 19, and further comprises a linker sequence selected from the group consisting of the sequences set forth in Table 20 wherein the linker is recombinantly fused between the VL and the VH and the TM1 and TM2 are recombinantly fused together by a short linker of hydrophilic amino acids selected from the group consisting of the sequences SGGGGS (SEQ ID NO: 1), GGGGS (SEQ ID NO: 2), GGS, and GSP; ii) the CCD is selected from the group consisting of the CCD of Table 6; iii) the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; and iv) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

In another embodiment, the targeted conjugate composition is configured according to the structure of formula VII:

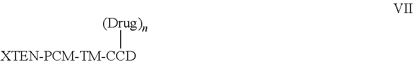

wherein: i) the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; ii) the CCD is selected from the group consisting of the CCD of Table 6; iii) the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; and v) the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD.

6. Multimeric Configurations of Compositions

In one aspect, the invention provides targeted conjugate compositions wherein different numbers of XTEN partners are joined by linkers in a numerically-defined configuration; e.g., dimeric, trimeric, tetrameric, or multimeric. As used herein, “precursor” is intended to include components used as reactants in a conjugation reaction leading to an intermediate or fmal composition, and includes but is not limited to XTEN segments of any length (including the XTEN of Tables 10 and 11), XTEN-crosslinkers, XTEN-payload-crosslinker segments, CCD-cross-linkers, CCD-payloads, CCD-XTEN-crosslinkers, payloads with reactive groups, linkers, and other such components described herein.

In some embodiments, the invention provides conjugates in which two XTEN precursor segments are linked by a divalent cross-linker, resulting in a divalent configuration, such as shown in FIGS. 15-17. In one embodiment of the divalent XTEN-conjugate, each XTEN-conjugate can be a monomeric fusion protein further comprising a targeting moiety, a CCD, a PCM, and a biologically active peptide or polypeptide, wherein each fusion protein precursor segment is linked to the divalent linker by the alpha-amino group of the N-terminus, resulting in the divalent conjugate. In another embodiment of the divalent XTEN-conjugate, each XTEN precursor segment is a monomeric fusion protein comprising a targeting moiety, a CCD, a PCM, and a biologically active peptide or polypeptide, wherein each fusion protein is linked to the divalent linker at the C-terminus, resulting in the divalent conjugate. In another embodiment of the divalent XTEN-conjugate, each conjugate comprises one or more payloads (that can be a peptide, polypeptide or a drug) conjugated to the CCD, a CCD-XTEN fusion, or to the XTEN, wherein each precursor is linked to the other precursor comprising one or more second, different payload molecules by a divalent linker at the N-terminus, resulting in the divalent conjugate. In the foregoing embodiments of the paragraph, different approaches may be used to create the precurors to be linked, such as conjugating a linker to a first precuror XTEN and then effecting a second reaction to join the precursor to the reactive group of the terminus of the second XTEN or CCD-XTEN precursor. Alternatively, one or both of the XTEN or CCD-XTEN termini can be modified as precurors that can then be joined by click chemistry or by other methods described or illustrated herein, leaving few or no residual atoms to bridge the intersection of the resulting conjugate. In another embodiment, two CCD-XTEN or XTEN precuror sequences are linked by a disulfide bridge using cysteines or thiol groups introduced at or near the termini of the precursor XTEN reactants, resulting in a divalent XTEN-conjugate. Using such methods, various configurations can be constructed. Exemplary configurations of such divalent conjugates follow.

In one embodiment, the invention provides a targeted conjugate composition having the structure of formula VIII

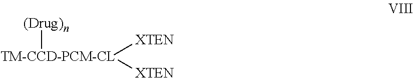

wherein independently for each occurrence the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; the CCD is selected from the group consisting of the CCD of Table 6; the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; CL is a trimeric cross-linker selected from Table 24, each XTEN is identical wherein the XTEN has at least 90%, or 91%, or 92%, or 93%, or 94%, or 95%, or 96%, or 97%, or 98%, or 99% identity or is identical to a sequence set forth in Table 10; and the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD. In the foregoing embodiment, the XTEN can be linked to the fusion protein using a trimeric cross-linker including, but not limited to the cross-linkers of Table 24.

The invention further provides XTEN-linker and XTEN-linker payload conjugates with a tetrameric configuration. In one embodiment, the invention provides conjugates in which three XTEN sequences are linked by a tetraravalent linker, resulting in a tetrarameric configuration. In one embodiment, the invention provides a targeted conjugate composition having the structure of formula IX

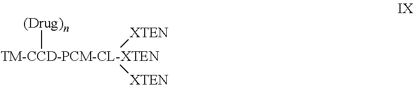

wherein independently for each occurrence the TM is an scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; the CCD is selected from the group consisting of the CCD of Table 6; the PCM is selected from the group consisting of the sequences set forth in Table 8; iv) the XTEN has at least 90%, or 91%, or 92%, or 93%, or 94%, or 95%, or 96%, or 97%, or 98%, or 99% identity or is identical to a sequence set forth in Table 10; CL is a tetravalent cross-linker; each XTEN is identical wherein the XTEN has at least 90%, or 91%, or 92%, or 93%, or 94%, or 95%, or 96%, or 97%, or 98%, or 99% identity or is identical to a sequence set forth in Table 10; and the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD. Non-limiting examples of tetravalent linkers include a tetraravalent-thiol, a quadravalent-N-maleimide linker such as described in U.S. Pat. No. 7,524,821.

In another embodiment, the invention provides a targeted conjugate composition having the structure of formula X

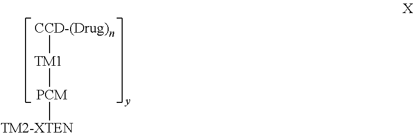

wherein independently for each occurrence the TM1 is a first scFv comprising a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences set forth in Table 20 wherein the linker is recombinantly fused between the VL and the VH; the TM2 is a second scFv, different from the first scFv, wherein the TM2 comprises a VL and a VH sequence, wherein each VL and VH is derived from an antibody of Table 19 or has at least 90%, or 91%, or 92%, or 93%, or 94%, or 95%, or 96%, or 97%, or 98%, or 99% identity or is identical to a VL and a VH from an antibody selected from the group consisting of the VL and VH sequences set forth in Table 19, and further comprises a linker sequence selected from the group consisting of the sequences of Table 20 wherein the linker is recombinantly fused between the VL and the VH; the CCD is selected from the group consisting of the CCD of Table 6; the PCM is selected from the group consisting of the PCM of Table 8; the XTEN is a cysteine-engineered XTEN having at least 90%, or 91%, or 92%, or 93%, or 94%, or 95%, or 96%, or 97%, or 98%, or 99% identity or is identical to a sequence set forth in Table 11; the drug is selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin; wherein n is equal to the number of cysteine residues of the CCD; and y is an integer between 3 and 10, inclusive, equal to the number of cysteine residues of the XTEN.

It is specifically contemplated that each the scFv of the TM of the embodiments of formulae I-X have VL and VH that can be configured, from the N-terminus to the C-terminus, as VH-linker-VL or VL-linker-VH and that the TM1 and TM2 of the embodiments of formula V and formula VI can each independently be configured as VH-linker-VL or VL-linker-VH.

7. Multivalent Configurations with Four or More XTEN

Using XTEN of Table 11, compositions are contemplated containing three or more XTEN-conjugate molecules linked to the cysteine-engineered backbone, in which fusion proteins of PCM, TM, and CCD (with linked payload drugs) are conjugated to the cysteine residues of the XTEN resulting in a “comb” multivalent configuration. In one embodiment, the multivalent configuration conjugate composition is created by reacting the N-terminus of the PCM of the foregoing fusion protein to the cysteine-engineered XTEN with a linker appropriate for reaction with the cysteine-engineered XTEN, resulting in the final product. In the embodiments, the valency of the final product is controlled by the number of reactive cysteine groups incorporated into the XTEN. Additionally, it is contemplated that the fmal product can be designed to locate a second targeting moiety on the N- or C-terminus of the XTEN, which improves interactions with its ligand on the target cell. Representative schematic examples of such comb configurations are shown in FIGS. 37 and 39.

8. Bispecific Payload Configurations on Monomer XTEN Backbone

In another aspect, the invention provides XTEN-conjugates containing two different payload molecules linked to a single cysteine-engineered XTEN backbone, resulting in a bivalent conjugate. In one embodiment, the bivalent configuration conjugate is created by reacting the engineered XTEN, such as those specifically provided in Table 11, with a first targeted conjugate composition with one or more molecules of a first attached payload drug with a cross-linker appropriate for reaction with the cysteine-engineered XTEN, followed by a second reaction with a second targeted conjugate composition with one or more molecules of a second, different attached payload drug with a cross-linker appropriate for reaction with the lysine-engineered XTEN, resulting in the final product. The number and location of linked targeted conjugate compositions is controlled by the design of the engineered XTEN, with the placement of the reactive thiol or amino group being determinative. In one embodiment, the bivalent conjugate comprises a single molecule of a first targeted conjugate compositions with one or molecules of a first payload and a single molecule of a second targeted conjugate compositions with one or more molecules of a second payload linked to the respective cysteine and lysine residues of the engineered XTEN. In another embodiment, the bivalent conjugate comprises one, or two, or three, or more molecules of a first targeted conjugate compositions with one or molecules of a first payload linked to cysteine residues of the cysteine-lysine-engineered XTEN and a single molecule of a second targeted conjugate compositions with one or molecules of a second payload linked to a single lysine of the cysteine-lysine-engineered XTEN. In another embodiment, the bivalent conjugate comprises one, or two, or three, or four, or five molecules of a first payload and one, or two, or three, or four, or five molecules of a second payload linked to the cysteine-lysine-engineered XTEN by linkers.

In another embodiment, the bivalent configuration conjugate is created by reacting the cysteine- and lysine-engineered XTEN, such as those of Table 11, with a first linker appropriate for reaction with the cysteine-engineered XTEN, followed by a second reaction with a linker appropriate for reaction with the lysine-engineered XTEN, then reacting the XTEN-crosslinker backbone with a first payload with a thiol reactive group capable of reacting with the first linker, followed by a reaction of a second payload with an amino group capable of reacting with the second cross-linker, resulting in the final product.

9. Libraries of XTEN-Payload Configurations

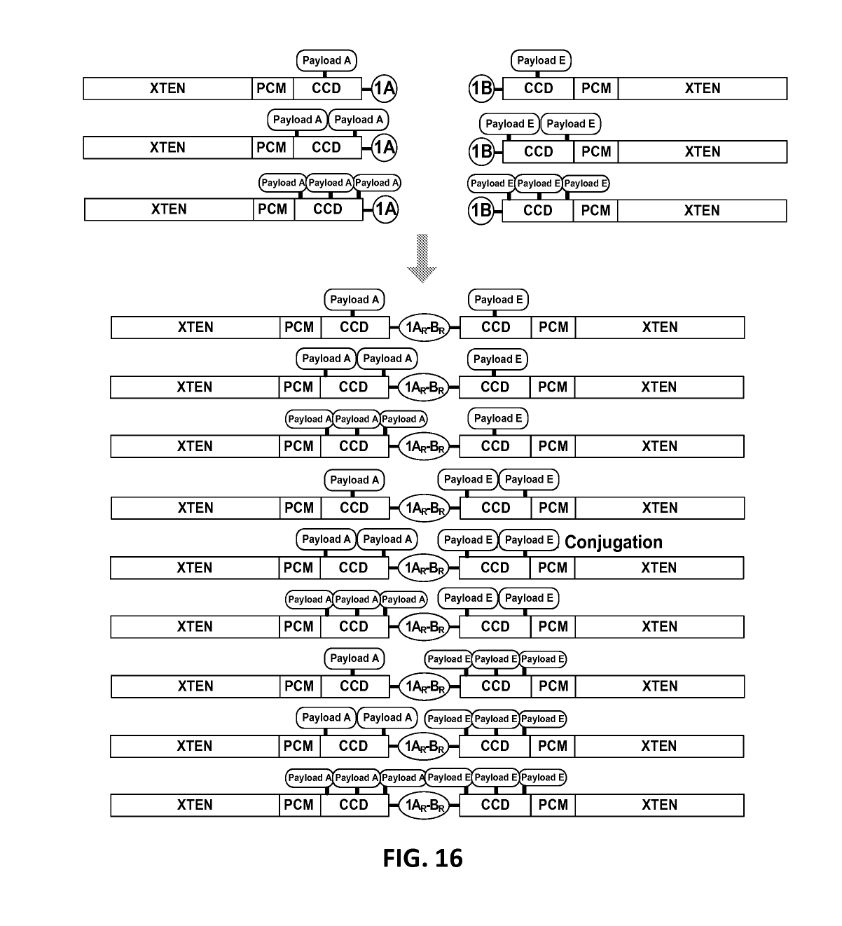

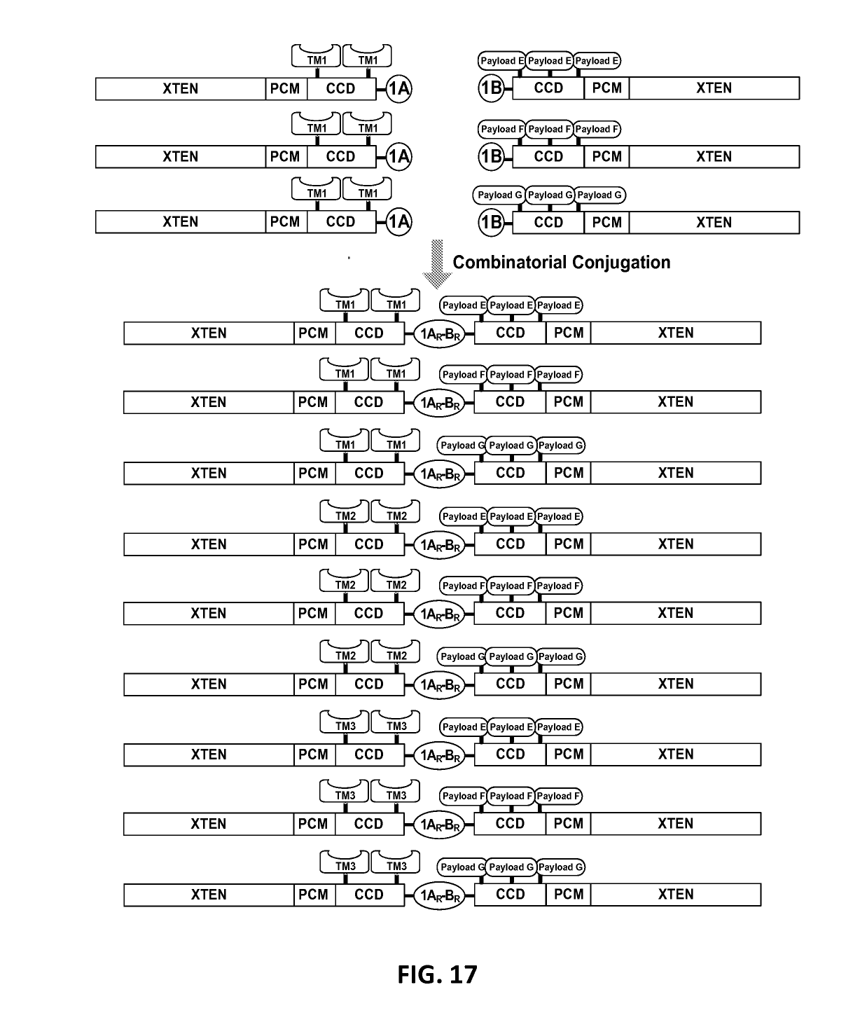

In another aspect, the invention provides libraries of XTEN-payload conjugate precursors, methods to make the libraries, and methods to combine the library precursors in a combinatorial approach, as illustrated in FIGS. 15-16, to achieve optimal combinations of, as well as the optimal ratio of payloads. In one embodiment, the invention provides a library of individual XTEN each linked to 1, or 2, or 3, or 4, or 5, or 6, or 7, or 8, or 9, or 10 or more or more molecules of a given payload, including those described herein, to create the library of XTEN-payload precursors. In the method, a series of XTEN-payload precursors to be linked are further conjugated to a linker, and then is subsequently mixed and reacted with the other XTEN-payload precursors capable of reacting with the linker under conditions to effect the conjugation, resulting in a library of the various permutations and ratios of payloads linked to XTEN in configurations described herein. Such a library is then screened in an in vitro or in vivo assay suitable to assess a parameter in a given clinical indication (e.g., cancer, metabolic disorder, diabetes) in order to determine those compositions providing the optimal response or action. In one exemplary embodiment, one category of precursor includes various targeting moieties, such as antibody fragments or scFv (e.g., of or derived from the antibodies of Table 19) with binding affinity to a tumor-associated antigens or ligands of Table 2, Table 3, Table 4, Table 18, or Table 19, and the second category of precursor is one or more payloads, such as a cytotoxic drug or a payload chosen from Tables 14-17. Each category of precursor to be linked is further conjugated to a linker, and, as illustrated in FIG. 17, is subsequently mixed and reacted with the other XTEN-payload precursors capable of reacting with the linker under conditions to effect the conjugation, resulting in a library of the various targeting moieties and drug permutations in varying ratios to each other. The XTEN-conjugates are designed to permit fixed ratios of one payload to another; e.g., is 1:1, or 1:1.5, or 1:2, or 1:3, or 2:3, or 1:4, or 1:5 or 1:9 in the case of two different payloads. Similar ranges of ratios would be applied for library conjugates comprising 3, 4, 5 or more payloads. In one embodiment of the foregoing, and as illustrated in FIG. 37, the conjugates further comprise one or more peptidyl cleavage moieties (PCM) between the XTEN backbone and the XTEN-payload component wherein the PCM is a substrate for a protease associated with the target tissue that is the ligand of the targeting moiety wherein the binding of the targeting moiety to the ligand brings the conjugate into proximity to the protease, resulting in the release of the XTEN-payload component and enhanced killing or an enhanced biological effect on the target tissue compared to a composition lacking in said PCM and targeting moiety. In such cases, the released payload may act directly at the surface of the tissue or may be internalized and further degraded, resulting in release of the payload, such as the cytotoxic drugs described herein.

### VII). Dosage Forms and Pharmaceutical Compositions

The present invention provides pharmaceutical compositions comprising targeted conjugate compositions of the disclosure. In one embodiment, the pharmaceutical composition comprises an targeted conjugate composition selected from the various embodiments described herein, and at least one pharmaceutically acceptable carrier.

In another aspect, the present invention provides bolus doses or dosage forms comprising a targeted conjugate composition described herein. In one embodiment, the bolus dose or dosage of a targeted conjugate composition comprises a therapeutically effective bodyweight adjusted bolus dose for a human patient.

In other embodiments, the bolus dose or dosage is (i) for use in treating cancer in a subject in need; and/or (ii) formulated for subcutaneous administration. In one embodiment, the bolus dose or dosage form is a pharmaceutical composition comprising a targeted conjugate composition of any of the embodiments disclosed herein and a pharmaceutically acceptable carrier.

In another embodiment, the invention provides kits, comprising packaging material and at least a first container comprising the pharmaceutical composition of the foregoing embodiment and a label identifying the pharmaceutical composition and storage and handling conditions, and optionally a sheet of instructions for the preparation and/or administration of the pharmaceutical compositions to a subject.

The invention provides a method of preparing a pharmaceutical composition, comprising the step of combining a subject targeted conjugate composition of the embodiments with at least one pharmaceutically acceptable carrier into a pharmaceutically acceptable formulation. The targeted conjugate compositions of the present invention can be formulated according to known methods to prepare pharmaceutically useful compositions, whereby the targeted conjugate composition is combined in admixture with a pharmaceutically acceptable carrier vehicle, such as aqueous solutions or buffers, pharmaceutically acceptable suspensions and emulsions. Examples of non-aqueous solvents include propyl ethylene glycol, polyethylene glycol and vegetable oils. Therapeutic formulations are prepared for storage by mixing the active ingredient having the desired degree of purity with optional physiologically acceptable carriers, excipients or stabilizers, as described in Remington's Pharmaceutical Sciences 16th edition, Osol, A. Ed. (1980), in the form of lyophilized formulations or aqueous solutions. The pharmaceutical compositions can be administered by any suitable means or route, including subcutaneously, subcutaneously by infusion pump, intramuscularly, and intravenously. It will be appreciated that the preferred route will vary with the disease and age of the recipient, and the severity of the condition being treated. Osmotic pumps may be used as slow release agents in the form of tablets, pills, capsules or implantable devices. Syringe pumps may also be used as slow release agents. Such devices are described in U.S. Pat. Nos. 4,976,696; 4,933,185; 5,017,378; 6,309,370; 6,254,573; 4,435,173; 4,398,908; 6,572,585; 5,298,022; 5,176,502; 5,492,534; 5,318,540; and 4,988,337, the contents of which are incorporated herein by reference. One skilled in the art, considering both the disclosure of this invention and the disclosures of these other patents could produce a syringe pump for the extended release of the compositions of the present invention.

In another embodiment, the invention provides an targeted conjugate composition of any of the embodiments described herein for use in making a medicament useful for the treatment of a condition including, but not limited a cancer or an inflammatory condition.

### VIII). Methods of Treatment

The invention provides a method of treating a disease in a subject, comprising administering to the subject a therapeutically effective effective amount of the targeted conjugate composition of any of the foregoing embodiments to a subject in need thereof. In one embodiment, the targeted conjugate composition of the method comprises a single type of payload selected from Tables 14-17. In another embodiment, the method comprises administering to the subject a therapeutically effective effective amount of a targeted conjugate composition selected from the group consisting of the constructs set forth in Table 5. In another embodiment, the method comprises administering to the subject a therapeutically effective effective amount of a targeted conjugate composition selected from the group consisting of the constructs set forth in the Examples.

In other embodiments, the method comprises administering to a human patient with cancer at least two therapeutically effective bodyweight adjusted bolus doses of a targeted conjugate composition of any of the embodiments disclosed herein. In one embodiment of the foregoing, the method comprises administering to a human patient with cancer at least two therapeutically effective bodyweight adjusted bolus doses of a targeted conjugate composition selected from the group consisting of the constructs set forth in Table 5, wherein said administration of said bolus doses is separated by at least about 7 days, at least about 10 days, at least about 14 days, at least about 21 days, at least about 28 days, or at least about monthly.

In another embodiment, the method comprises administering to a human patient with cancer at least two therapeutically effective bodyweight adjusted bolus doses of a targeted conjugate composition selected from the group consisting of the constructs set forth in Table 5, wherein said therapeutically effective bodyweight adjusted bolus dose is selected from the group consisting of at least about 0.05 mg/kg, at least about 0.1 mg/kg, at least about 0.2 mg/kg, at least about 0.4 mg/kg, at least about 0.8 mg/kg, at least about 1.0 mg/kg, at least about 1.2 mg/kg, at least about 1.4 mg/kg, at least about 1.6 mg/kg, at least about 1.8 mg/kg, at least about 2.0 mg/kg, at least about 2.2 mg/kg, at least about 2.4 mg/kg, at least about 2.6 mg/kg, at least about 2.7 mg/kg, at least about 2.8 mg/kg, at least 3.0 mg/kg, at least 4.0 mg/kg, at least about 5.0 mg/kg, at least about 6.0 mg/kg, at least about 7.0 mg/kg, at least about 10 mg/kg, or at least about 15 mg/kg.

In the methods of treatment, the payload of the targeted conjugate composition is one that is known in the art to have a beneficial effect when administered to a subject with a particular disease or condition. In one embodiment, the payload(s) of the composition mediate their therapeutic effect via a cytoxic effect on a cell of a target tissue. In the foregoing embodiments of the paragraph, the method is useful in treating or ameliorating or preventing a disease selected from cancer, cancer supportive care, or inflammation, autoimmune disease, infectious diseases, metabolic disease, musculoskeletal disease, nephrology disorders, ophthalmologic diseases, pain, and respiratory diseases associated with inflammation. With greater particularity, the cancer is selected from breast cancer, ER/PR+ breast cancer, Her2+ breast cancer, triple-negative breast cancer, liver carcinoma, lung cancer, non-small cell lung cancer, mesothelioma, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, adenocarcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, and pancreatic cancer, leukemia, acute myeloid leukemia (AML), chronic myeloid leukemia (PCML), acute lymphocytic leukemia (ALL), chronic lymphocytic leukemia (CLL), T-cell acute lymphoblastic leukemia, lymphoblastic disease, multiple myeloma, Hodgkin's lymphoma, and non-Hodgkin's lymphoma.

In some embodiments of the method of treatment, the targeted conjugate composition can be administered subcutaneously, intramuscularly, or intravenously. In one embodiment, the composition is administered using a therapeutically effective amount. In one embodiment, administration of two or more consecutive doses of the therapeutically effective amount results in a gain in time spent within a therapeutic window for the composition compared to the payload not linked to the targeted conjugate composition and administered using comparable doses to a subject. The gain in time spent within the therapeutic window can be at least three-fold longer than unmodified payload, or alternatively, at least four-fold, or five-fold, or six-fold, or seven-fold, or eight-fold, or nine-fold, or at least 10-fold, or at least 20-fold, or at least about 30-fold, or at least about 50-fold, or at least about 100-fold longer than the corresponding payload or payloads not linked to XTEN.

In one embodiment of the method of treatment, a smaller moles/kg amount of at least about two-fold less, or at least about three-fold less, or at least about four-fold less, or at least about five-fold less, or at least about six-fold less, or at least about eight-fold less, or at least about 10-fold less of the targeted conjugate composition or a pharmaceutical composition comprising the targeted conjugate composition is administered to a subject in need thereof in comparison to the corresponding payload(s) not linked to the targeted conjugate composition under a dose regimen needed to maintain a therapeutic effect. In some embodiments of the method, the therapeutic effect is a measured parameter, clinical symptom or endpoint known in the art to be associated with the underlying condition of the subject to be treated or prevented such as, but not limited to, presence or concentration of a cancer marker, size of a tumor, tumor stasis, numbers of tumors, tumor necrosis, body weight, cytokine levels, blood parameters, pain, time-to-progression of the cancer, time-to-relapse, time-to-discovery of local recurrence, time-to-discovery of regional metastasis, time-to-discovery of distant metastasis, time-to-onset of symptoms, hospitalization, time-to-increase in pain medication requirement, time-to-requirement of salvage chemotherapy, time-to-requirement of salvage surgery, time-to-requirement of salvage radiotherapy, time-to-treatment failure, and time of survival. In the foregoing embodiment, the time required to maintain the therapeutic effect is at least about 21 days, or at least about 30 days, or at least about one month, at least about 45 days, at least about 60 days, at least about 90 days, or at least about 120 days.

In another embodiment of the method of treatment, a smaller moles/kg amount of at least about two-fold less, or at least about three-fold less, or at least about four-fold less, or at least about five-fold less, or at least about six-fold less, or at least about eight-fold less, or at least about 10-fold less of the targeted conjugate composition or a pharmaceutical composition comprising the targeted conjugate composition is administered to a subject in need thereof in comparison to the corresponding payload(s) not linked to the targeted conjugate composition under a dose regimen needed to maintain a comparable area under the curve as the corresponding moles/kg amount of the payload(s) not linked to the targeted conjugate composition needed to maintain a therapeutic effect. In another embodiment, the targeted conjugate composition or a pharmaceutical composition comprising the conjugate requires less frequent administration for routine treatment of a subject, wherein the dose of targeted conjugate composition or pharmaceutical composition is administered about every four days, about every seven days, about every 10 days, about every 14 days, about every 21 days, or about monthly to the subject, and the targeted conjugate composition achieves a comparable area under the curve as the corresponding payload(s) not linked to the targeted conjugate composition and administered to the subject. In yet other embodiments, an accumulatively smaller amount of about 5%, or about 10%, or about 20%, or about 40%, or about 50%, or about 60%, or about 70%, or about 80%, or about 90% less of moles/kg of the targeted conjugate composition is administered to a subject in comparison to the corresponding amount of the payload(s) not linked to the targeted conjugate composition under a dose regimen needed to maintain an effective blood concentration, yet the conjugate achieves at least a comparable area under the curve as the corresponding payload(s) not linked to the targeted conjugate composition. The accumulatively smaller amount is measure for a period of at least about one week, or at least about 14 days, or at least about 21 days, or at least about 30 days, or at least about one month.

In one embodiment, the invention provides a method of treating a cancer cell in vitro, comprising administering to a culture of a cancer cell a composition comprising an effective amount of an targeted conjugate composition, comprising a targeting moiety directed to a target of Table 2 or Table 3, Table 4, Table 18, or Table 19. and one or more payloads selected from the compounds of Tables 14-17. In another embodiment, the invention provides a method of treating a cancer in a subject, comprising administering to the subject a pharmaceutical composition comprising an effective amount of an targeted conjugate composition comprising a targeting moiety directed to a target of Table 2, or Table 3, or Table 4, Table 18 or Table 19 and one or more payloads selected from the compounds of Tables 14-17. In another embodiment of the method, the cancer is selected from the group consisting of breast cancer, ER/PR+ breast cancer, Her2+ breast cancer, triple-negative breast cancer, liver carcinoma, lung cancer, non-small cell lung cancer, mesothelioma, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, adenocarcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, pancreatic cancer, leukemia, acute myeloid leukemia (AML), chronic myeloid leukemia (PCML), acute lymphocytic leukemia (ALL), chronic lymphocytic leukemia (CLL), T-cell acute lymphoblastic leukemia, lymphoblastic disease, multiple myeloma, Hodgkin's lymphoma and non-Hodgkin's lymphoma. In another embodiment of the method, the administration results in at least a 10%, or 20%, or 30%, or 40%, or 50%, or 60%, or 70%, or 80%, or 90% greater improvement of at least one, two, or three parameters associated with a cancer compared to an untreated subject wherein the parameters are selected from the group consisting of response rate as defined by the Response Evaluation Criteria in Solid Tumors (RECIST), time-to-progression of the cancer (relapse), discovery of local recurrence, discovery of regional metastasis, discovery of distant metastasis, onset of symptoms, hospitalization, increase in pain medication requirement, requirement of salvage chemotherapy, requirement of salvage surgery, requirement of salvage radiotherapy, time-to-treatment failure, and increased time of survival.

In another aspect, the invention provides a regimen for treating a subject with a disease, said regimen comprising a composition comprising a targeted conjugate composition of any of the embodiments described herein. In one embodiment of the regimen, the regimen further comprises the step of determining the amount of pharmaceutical composition comprising the targeted conjugate composition needed to achieve a therapeutic effect in the patient.

The invention provides targeted conjugate composition comprising a treatment regimen for a diseased subject comprising administering a pharmaceutical composition comprising a conjugate of any of the embodiments described herein in two or more successive doses adminitered at an effective amount, wherein the adminstration results in the improvement of at least one parameter associated with the disease.

In some embodiments, the invention provides a method of treating a disease, comprising a regimen of administering one, or two, or three, or four or more therapeutically effective doses of a pharmaceutical composition comprising an embodiment of a targeted conjugate composition to a subject in need thereof. In one embodiment of the method, the disease is selected from the group consisting of breast cancer, ER/PR+ breast cancer, Her2+ breast cancer, triple-negative breast cancer, liver carcinoma, lung cancer, non-small cell lung cancer, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, and pancreatic cancer. In another embodiment of the method, the administered pharmaceutical composition comprises a targeting moiety wherein the targeting moiety has specific binding affinity for a tumor of the disease. In another embodiment of the method, the administered pharmaceutical composition comprises a targeting moiety wherein the targeting moiety has specific binding affinity for a tumor associated antigen selected from the group consisting of the tumor associated antigens of Table 3. In another embodiment of the method, the administered doses result in a decrease in the tumor size in the subject of at least 10%, or at least 20%, or at least 30%, or at least 40%, or at least 50% or greater. In the foregoing embodiment, the decrease in tumor size is achieved within at least about 7 days, or at least about 10 days, or at least about 14 days, or at least about 21 days, or at least about 30 days after the administration. In another embodiment of the method, the administered doses result in tumor stasis in the subject, wherein statsis is achieved within at least about 7 days, or at least about 10 days, or at least about 14 days, or at least about 21 days, or at least about 30 days after the administration. In the foregoing embodiments of the paragraph the regimen comprises administration of the therapeutically effective dose every 7 days, or every 10 days, or every 14 days, or every 21 days, or monthly.

In another aspect, the invention provides a targeted conjugate composition for use in the preparation of a medicament for use in treating a disease in a subject. In one embodiment, the disclosure provides a targeted conjugate composition of any of the embodiments disclosed herein for use in the preparation of a medicament for use in treating a cancer in a subject. In the foregoing embodiment, the cancer is selected from the group consisting of breast cancer, ER/PR+ breast cancer, Her2+ breast cancer, triple-negative breast cancer, liver carcinoma, lung cancer, non-small cell lung cancer, mesothelioma, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, adenocarcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, pancreatic cancer, leukemia, acute myeloid leukemia (AML), chronic myeloid leukemia (PCML), acute lymphocytic leukemia (ALL), chronic lymphocytic leukemia (CLL), T-cell acute lymphoblastic leukemia, lymphoblastic disease, multiple myeloma, Hodgkin's lymphoma and non-Hodgkin's lymphoma.

### IX). Pharmaceutical Kits

In another aspect, the invention provides a kit to facilitate the use of the conjugate compositions. In some embodiment, the kit comprises a pharmaceutical composition provided herein, a container and a label or package insert on or associated with the container. Suitable containers include, for example, bottles, vials, syringes, etc., formed from a variety of materials such as glass or plastic. The container holds a pharmaceutical composition as a formulation that is effective for treating a subject and may have a sterile access port (for example the container may be an intravenous solution bag or a vial having a stopper pierceable by a hypodermic injection needle). The package insert can list the approved indications for the drug, instructions for the reconstitution and/or administration of the drug for the use for the approved indication, appropriate dosage and safety information, and information identifying the lot and expiration of the drug. In another embodiment of the foregoing, the kit can comprise a second container that can carry a suitable diluent for the pharmaceutical composition, the use of which will provide the user with the appropriate concentration to be delivered to the subject. In another embodiment, the kit comprises, in at least a first container: a first container: an amount of a conjugate composition drug sufficient to administer in treatment of a subject with a disease; an amount of a pharmaceutically acceptable carrier; a second container that can carry a suitable diluent for the subject composition, which will provide the user with the appropriate concentration of the pharmaceutical composition to be delivered to the subject; together with a label identifying the drug and storage and handling conditions, and/or a sheet of the approved indications for the drug and instructions for the reconstitution and/or administration of the drug for the use for the treatment of a approved indication, appropriate dosage and safety information, and information identifying the lot and expiration of the drug.

### X). The Nucleic Acids Sequences of the Invention

The present invention provides isolated polynucleic acids encoding the polypeptide components of the targeted conjugate compositions and sequences complementary to polynucleic acid molecules encoding the polypeptide components of the targeted conjugate compositions. In some embodiments, the invention provides polynucleic acids encoding the targeted conjugate composition of any of the embodiments described herein, or the complement of the polynucleic acid.

In other embodiments, the invention provides polynucleic acids encoding the fusion proteins of targeting moieties, CCD, PCM and XTEN fused as a single polypeptide of any of the embodiments described herein, or the complement of the polynucleic acids. In one embodiment, the polynucleic acids encodes the protein components selected from the group consisting of the constructs of Table 5, or the complement of the polynucleic acid.

In one embodiment, the invention encompasses methods to produce polynucleic acids encoding the polypeptides of the targeted conjugate compositions, or sequences complementary to the polynucleic acids, including homologous variants thereof. In general, the methods include producing a polynucleotide sequence coding for the polypeptides of the targeted conjugate compositions and expressing the resulting gene product and assembling nucleotides encoding the components, ligating the components in frame, incorporating the encoding gene into an expression vector appropriate for a host cell, transforming the appropriate host cell with the expression vector, and culturing the host cell under conditions causing or permitting the resulting fusion protein to be expressed in the transformed host cell, thereby producing the fusion protein polypeptide, which is recovered by methods described herein or by standard protein purification methods known in the art. In one embodiment of the foregoing, the host cell is a prokaryonte cell. In another embodiment, the host cell is E. coli. Standard recombinant techniques in molecular biology are used to make the polynucleotides and expression vectors of the present invention.

In accordance with the invention, nucleic acid sequences that encode polypeptides of the targeted conjugate compositions (or its complement) are used to generate recombinant DNA molecules that direct the expression in appropriate host cells. Several cloning strategies are suitable for performing the present invention, many of which are used to generate a construct that comprises a gene coding for a composition of the present invention, or its complement. In one embodiment, the cloning strategy is used to create a gene that encodes a polypeptide of a targeted conjugate composition that comprises nucleotides encoding the polypeptide that is used to transform a host cell for expression of the polypeptide of the targeted conjugate composition. In another embodiment, the cloning strategy is used to create a gene that encodes a protein payload that comprises nucleotides encoding the payload that is used to transform a host cell for expression of the payload composition for conjugation to the polypeptides of the targeted conjugate composition.

In one approach, a construct is first prepared containing the DNA sequence corresponding to a polypeptide of the targeted conjugate composition. Exemplary methods for the preparation of such constructs are described in the Examples. The construct is then used to create an expression vector suitable for transforming a host cell, such as a prokaryotic host cell (e.g., E. coli) for the expression and recovery of the XTEN. Exemplary methods for the creation of expression vectors, the tranformation of host cells and the expression and recovery of the polypeptides of the subject compositions are described in the Examples.

The gene encoding for the polypeptides of the targeted conjugate composition can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate short sequences of polynucleotides encoding the individual component genes of a desired sequence. Genes encoding polypeptides of the targeted conjugate compositions are assembled from oligonucleotides using standard techniques of gene synthesis. The gene design can be performed using algorithms that optimize codon usage and amino acid composition. The resulting genes are then assembled with genes encoding payload peptide or polypeptide of the targeted conjugate composition, and the resulting genes used to transform a host cell and produce and recover the polypeptide of the targeted conjugate composition for evaluation of its properties, as described herein.

The resulting polynucleotides encoding the polypeptides of the targeted conjugate compositions can then be individually cloned into an expression vector. The nucleic acid sequence is inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated.

The invention provides for the use of plasmid expression vectors containing replication and control sequences that are compatible with and recognized by the host cell, and are operably linked to the gene encoding the polypeptide for controlled expression of the polypeptide. The vector ordinarily carries a replication site, as well as sequences that encode proteins that are capable of providing phenotypic selection in transformed cells. Such vector sequences are well known for a variety of bacteria, yeast, and viruses. Useful expression vectors that can be used include, for example, segments of chromosomal, non-chromosomal and synthetic DNA sequences. “Expression vector” refers to a DNA construct containing a DNA sequence that is operably linked to a suitable control sequence capable of effecting the expression of the DNA encoding the polypeptide in a suitable host. The requirements are that the vectors are replicable and viable in the host cell of choice. Low- or high-copy number vectors may be used as desired.

Suitable vectors include, but are not limited to, derivatives of SV40 and pcDNA and known bacterial plasmids such as col EI, pCRl, pBR322, pMal-C2, pET, pGEX as described by Smith, et al., Gene 57:31-40 (1988), pMB9 and derivatives thereof, plasmids such as RP4, phage DNAs such as the numerous derivatives of phage I such as NM98 9, as well as other phage DNA such as M13 and filamentous single stranded phage DNA; yeast plasmids such as the 2 micron plasmid or derivatives of the 2 m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like. Yeast expression systems that can also be used in the present invention include, but are not limited to, the non-fusion pYES2 vector (Invitrogen), the fusion pYESHisA, B, C (Invitrogen), pRS vectors and the like. The control sequences of the vector include a promoter to effect transcription, an optional operator sequence to control such transcription, a sequence encoding suitable mRNA ribosome binding sites, and sequences that control termination of transcription and translation. The promoter may be any DNA sequence, which shows transcriptional activity in the host cell of choice and may be derived from genes encoding proteins either homologous or heterologous to the host cell. Promoters suitable for use in expression vectors with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)], all is operably linked to the DNA encoding CFXTEN polypeptides. Promoters for use in bacterial systems can also contain a Shine-Dalgarno (S. D.) sequence, operably linked to the DNA encoding CFXTEN polypeptides.

### XI). Concatenate Sequences of the Invention

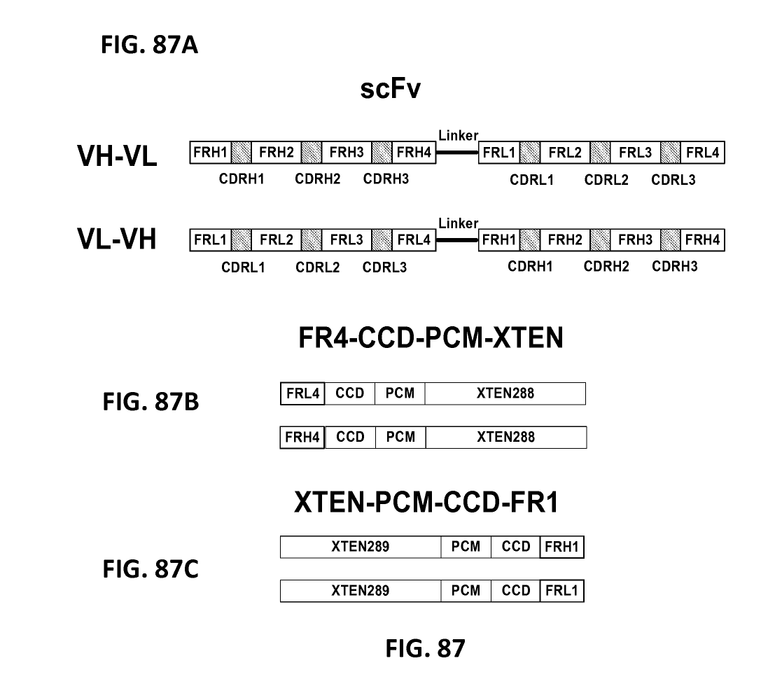

In another aspect, the invention provides compositions comprising fusion proteins of concatenates of polypeptide components of targeted conjugate compositions useful in making or assembling targeted conjugate compositions. As illustrated in FIGS. 87B and C, the contantenate sequences of the sequences of Tables 26 and 27 are fusion proteins having at least four different configurations, in an N-terminus to C-terminus configuration; 1) a FRH4 sequence, a CCD, a PCM, and a short XTEN sequence of 228 amino acids; 2) a FRL4 sequence, a CCD, a PCM, and a short XTEN sequence of 228 amino acids; 3) a short XTEN sequence of 229 amino acids, a PCM, a CCD, and a FRL1; and 4) a short XTEN sequence of 229 amino acids, a PCM, a CCD, and a FRH1. It is an object of the invention to provide fusion protein concatenates in compositions of targeted conjugate compositions in which a concatenate sequence is inserted into a fusion protein between a scFv of the embodiments disclosed herein (less the corresponding FR sequences WGQGTLVTVS (SEQ ID NO: 613), or TFGQGTKVEIK (SEQ ID NO: 614), or EVQLVESGGG (SEQ ID NO: 615), or DIQMTQSPSS (SEQ ID NO: 616)) and an XTEN of Table 10 or Table 11. The resulting fusion protein would then be conjugated to one or more payload drugs or biologics described herein, including but not limited to those of Tables 14-17, resulting in the targeted conjugate composition.

In one embodiment, the invention provides compositions comprising sequences exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 26. In another embodiment, the invention provides compositions comprising sequences exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 27.

In another embodiment, the invention provides compositions comprising a fusion protein having the components, in an N- to C-terminal orientation:1) a first region comprising a scFv derived from an antibody of Table 19 comprising a FRL1, CDRL1, FRL2, CDRL2, FRL3, CRL3, a FRL4, a linker from Table 20, FRH1, CDRH1, FRH2, CDRH2, FRH3, and CRH3 sequences; 2) a second region comprising a sequence exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence from Table 26; and 3) a third region comprising an XTEN exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 10 or Table 11.

In another embodiment, the invention provides compositions comprising a fusion protein having the components, in an N- to C-terminal orientation; 1) a first region comprising a scFv derived from an antibody of Table 19 comprising a FRH1, CDRH1, FRH2, CDRH2, FRH3, CRH3, a FRH4, a linker from Table 20, FRL1, CDRL1, FRL2, CDRL2, FRL3, and a CRL3 sequences; 2) a second region comprising a sequence exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence from Table 26; and 3) a third region comprising an XTEN exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 10 or Table 11.

In another embodiment, the invention provides compositions comprising a fusion protein having the components, in an N- to C-terminal orientation:1) a first region comprising an XTEN exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 10 or Table 11; 2) a second region comprising a sequence exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence from Table 27; and 3) a third region comprising a a scFv derived from an antibody of Table 19 comprising CDRL1, FRL2, CDRL2, FRL3, CRL3, a FRL4, a linker from Table 20, FRH1, CDRH1, FRH2, CDRH2, FRH3, CRH3 and FRH4 sequences.

In another embodiment, the invention provides compositions comprising a fusion protein having the components, in an N- to C-terminal orientation:1) a first region comprising an XTEN exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence selected from the group of sequences set forth in Table 10 or Table 11; 2) a second region comprising a sequence exhibiting at least about 90%, or at least about 91%, or at least about 92%, or at least about 93%, or at least about 94%, or at least about 95%, or at least about 96%, or at least about 97%, or at least about 98%, or at least about 99% identity, or is identical to a sequence from Table 27; and 3) a third region comprising a a scFv derived from an antibody of Table 19 comprising CDRH1, FRH2, CDRH2, FRH3, CRH3, a FRH4, a linker from Table 20, FRL1, CDRL1, FRL2, CDRL2, FRL3, CRL3 and FRH4 sequences.

In other embodiments, the invention provides a targeted conjugate composition comprising the fusion proteins of the paragraphs of this section with a drug or biologic linked to the cysteine residues wherein the drug or biologic is selected from the drugs or biologics of Tables 14-17. In one embodiment of the foregoing the drug or biologic is selected from the group consisting of group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), bortezomib, hTNF, I1-12, ranpirnase, hTNF, IL-12, ranpirnase, human ribonuclease (RNAse), Bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin.

TABLE 26
Concatenates of fusion protein components for targeted conjugate compositions

| | | SEQ ID |
|---|---|---|
| No. | Amino Acid Sequences of FR4-CCD-PCM-XTEN* | NO: |
| 1 | WGQGTLVTVSGSPGAGSCAGLSGRSDNHSPLGLAGSPGSPAGSPTSTEEGTSESATPES | 617 |
| | GPGTSTEPSEGSAPGSPAGSPTSTEEGTSTEPSEGSAPGTSTEPSEGSAPGTSESATPE | |
| | SGPGSEPATSGSETPGSEPATSGSETPGSPAGSPTSTEEGTSESATPESGPGTSTEPSE | |
| | GSAPGTSTEPSEGSAPGSPAGSPTSTEEGTSTEPSEGSAPGTSTEPSEGSAPGTSESAT | |
| | PESGPGTSTEPSEGSAPGTSESATPESGPGSEPATSGSETPGTSTEPSEGSAPGTSTEP | |
| | SEGSAPGTSESATPESGPGTSESATPESG | |
| 2 | WGQGTLVTVSGAPCGALSGRSDNHSPLGLAGSPGSPAGSPTSTEEGTSESATPESGPGT | 618 |
| | STEPSEGSAPGSPAGSPTSTEEGTSTEPSEGSAPGTSTEPSEGSAPGTSESATPESGPG | |
| | SEPATSGSETPGSEPATSGSETPGSPAGSPTSTEEGTSESATPESGPGTSTEPSEGSAP | |
| | GTSTEPSEGSAPGSPAGSPTSTEEGTSTEPSEGSAPGTSTEPSEGSAPGTSESATPESG | |
| | PGTSTEPSEGSAPGTSESATPESGPGSEPATSGSETPGTSTEPSEGSAPGTSTEPSEGS | |
| | APGTSESATPESGPGTSESATPESG | |
| 3 | WGQGTLVTVSGSPGAGSCAGSPTSTEEGTSESACSPEGPGTSTEPSEGSCGGLSGRSDN | 619 |
| | HSPLGLAGSPGSPAGSPTSTEEGTSESATPESGPGTSTEPSEGSAPGSPAGSPTSTEEG | |
| | TSTEPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPTSTE | |
| | EGTSTEPSEGSAPGTSTEPSEGSAPGTSESATPESGPGTSTEPSEGSAPGTSESATPES | |
| | GPGSEPATSGSETPGTSTEPSEGSAPGTSTEPSEGSAPGTSESATPESGPGTSESATPE | |
| | SG | |
| 4 | WGQGTLVTVSGAPCGAGCAGPCGPLSGRSDNHSPLGLAGSPGSPAGSPTSTEEGTSESA | 620 |
| | TPESGPGTSTEPSEGSAPGSPAGSPTSTEEGTSTEPSEGSAPGTSTEPSEGSAPGTSES | |
| | ATPESGPGSEPATSGSETPGSEPATSGSETPGSPAGSPTSTEEGTSESATPESGPGTST | |
| | EPSEGSAPGTSTEPSEGSAPGSPAGSPTSTEEGTSTEPSEGSAPGTSTEPSEGSAPGTS | |
| | ESATPESGPGTSTEPSEGSAPGTSESATPESGPGSEPATSGSETPGTSTEPSEGSAPGT | |
| | STEPSEGSAPGTSESATPESGPGTSESATPESG | |
| 5 | WGQGTLVTVSGSPGAGSCAGSPTSTEEGTSESACSPEGPGTSTEPSEGSCGPSPAGSPT | 621 |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASGLSGRSDNHSPLGLAGSPGSPAGS | |
| | PTSTEEGTSESATPESGPGTSTEPSEGSAPGSPAGSPTSTEEGTSTEPSEGSAPGTSTE | |
| | PSEGSAPGTSESATPESGPGSEPATSGSETPGSEPATSGSETPGSPAGSPTSTEEGTSE | |
| | SATPESGPGTSTEPSEGSAPGTSTEPSEGSAPGSPAGSPTSTEEGTSTEPSEGSAPGTS | |
| | TEPSEGSAPGTSESATPESGPGTSTEPSEGSAPGTSESATPESGPGSEPATSGSETPGT | |
| | STEPSEGSAPGTSTEPSEGSAPGTSESATPESGPGTSESATPESG | |
| 6 | WGQGTLVTVSGSPGAGSCTETSPSTCPTESPEACGSGSGSPCSPSGTEACSTSGSEGCS | 622 |
| | PSSTAPCGPTETEGCTTSSGPPCPESATSEGLSGRSDNHSPLGLAGSPGSPAGSPTSTE | |
| | EGTSESATPESGPGTSTEPSEGSAPGSPAGSPTSTEEGTSTEPSEGSAPGTSTEPSEGS | |
| | APGTSESATPESGPGSEPATSGSETPGSEPATSGSETPGSPAGSPTSTEEGTSESATPE | |
| | SGPGTSTEPSEGSAPGTSTEPSEGSAPGSPAGSPTSTEEGTSTEPSEGSAPGTSTEPSE | |
| | GSAPGTSESATPESGPGTSTEPSEGSAPGTSESATPESGPGSEPATSGSETPGTSTEPS | |
| | EGSAPGTSTEPSEGSAPGTSESATPESGPGTSESATPESG | |
| 7 | WGQGTLVTVSGSPGAGSCAGSPLGLAGSLSGRSDNHPGSPAGSPTSTEEGTSESATPES | 623 |
| | GPGTSTEPSEGSAPGSPAGSPTSTEEGTSTEPSEGSAPGTSTEPSEGSAPGTSESATPE | |
| | SGPGSEPATSGSETPGSEPATSGSETPGSPAGSPTSTEEGTSESATPESGPGTSTEPSE | |
| | GSAPGTSTEPSEGSAPGSPAGSPTSTEEGTSTEPSEGSAPGTSTEPSEGSAPGTSESAT | |
| | PESGPGTSTEPSEGSAPGTSESATPESGPGSEPATSGSETPGTSTEPSEGSAPGTSTEP | |
| | SEGSAPGTSESATPESGPGTSESATPESG | |
| 8 | WGQGTLVTVSGAPCGASPLGLAGSLSGRSDNHPGSPAGSPTSTEEGTSESATPESGPGT | 624 |
| | STEPSEGSAPGSPAGSPTSTEEGTSTEPSEGSAPGTSTEPSEGSAPGTSESATPESGPG | |
| | SEPATSGSETPGSEPATSGSETPGSPAGSPTSTEEGTSESATPESGPGTSTEPSEGSAP | |
| | GTSTEPSEGSAPGSPAGSPTSTEEGTSTEPSEGSAPGTSTEPSEGSAPGTSESATPESG | |
| | PGTSTEPSEGSAPGTSESATPESGPGSEPATSGSETPGTSTEPSEGSAPGTSTEPSEGS | |
| | APGTSESATPESGPGTSESATPESG | |
| 9 | WGQGTLVTVSGSPGAGSCAGSPTSTEEGTSESACSPEGPGTSTEPSEGSCGGSPLGLAG | 625 |
| | SLSGRSDNHPGSPAGSPTSTEEGTSESATPESGPGTSTEPSEGSAPGSPAGSPTSTEEG | |
| | TSTEPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPTSTE | |
| | EGTSTEPSEGSAPGTSTEPSEGSAPGTSESATPESGPGTSTEPSEGSAPGTSESATPES | |
| | GPGSEPATSGSETPGTSTEPSEGSAPGTSTEPSEGSAPGTSESATPESGPGTSESATPE | |
| | SG | |
| 10 | WGQGTLVTVSGAPCGAGCAGPCGPSPLGLAGSLSGRSDNHPGSPAGSPTSTEEGTSESA | 626 |
| | TPESGPGTSTEPSEGSAPGSPAGSPTSTEEGTSTEPSEGSAPGTSTEPSEGSAPGTSES | |
| | ATPESGPGSEPATSGSETPGSEPATSGSETPGSPAGSPTSTEEGTSESATPESGPGTST | |
| | EPSEGSAPGTSTEPSEGSAPGSPAGSPTSTEEGTSTEPSEGSAPGTSTEPSEGSAPGTS | |
| | ESATPESGPGTSTEPSEGSAPGTSESATPESGPGSEPATSGSETPGTSTEPSEGSAPGT | |
| | STEPSEGSAPGTSESATPESGPGTSESATPESG | |
| 11 | WGQGTLVTVSGSPGAGSCAGSPTSTEEGTSESACSPEGPGTSTEPSEGSCGPSPAGSPT | 627 |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASGSPLGLAGSLSGRSDNHPGSPAGS | |
| | PTSTEEGTSESATPESGPGTSTEPSEGSAPGSPAGSPTSTEEGTSTEPSEGSAPGTSTE | |
| | PSEGSAPGTSESATPESGPGSEPATSGSETPGSEPATSGSETPGSPAGSPTSTEEGTSE | |
| | SATPESGPGTSTEPSEGSAPGTSTEPSEGSAPGSPAGSPTSTEEGTSTEPSEGSAPGTS | |
| | TEPSEGSAPGTSESATPESGPGTSTEPSEGSAPGTSESATPESGPGSEPATSGSETPGT | |
| | STEPSEGSAPGTSTEPSEGSAPGTSESATPESGPGTSESATPESG | |
| 12 | WGQGTLVTVSGSPGAGSCTETSPSTCPTESPEACGSGSGSPCSPSGTEACSTSGSEGCS | 628 |
| | PSSTAPCGPTETEGCTTSSGPPCPESATSEGSPLGLAGSLSGRSDNHPGSPAGSPTSTE | |
| | EGTSESATPESGPGTSTEPSEGSAPGSPAGSPTSTEEGTSTEPSEGSAPGTSTEPSEGS | |
| | APGTSESATPESGPGSEPATSGSETPGSEPATSGSETPGSPAGSPTSTEEGTSESATPE | |
| | SGPGTSTEPSEGSAPGTSTEPSEGSAPGSPAGSPTSTEEGTSTEPSEGSAPGTSTEPSE | |
| | GSAPGTSESATPESGPGTSTEPSEGSAPGTSESATPESGPGSEPATSGSETPGTSTEPS | |
| | EGSAPGTSTEPSEGSAPGTSESATPESGPGTSESATPESG | |
| 13 | WGQGTLVTVSGSPGAGSCAGSPLGLSGRSDNHPGSPAGSPTSTEEGTSESATPESGPGT | 629 |
| | STEPSEGSAPGSPAGSPTSTEEGTSTEPSEGSAPGTSTEPSEGSAPGTSESATPESGPG | |
| | SEPATSGSETPGSEPATSGSETPGSPAGSPTSTEEGTSESATPESGPGTSTEPSEGSAP | |
| | GTSTEPSEGSAPGSPAGSPTSTEEGTSTEPSEGSAPGTSTEPSEGSAPGTSESATPESG | |
| | PGTSTEPSEGSAPGTSESATPESGPGSEPATSGSETPGTSTEPSEGSAPGTSTEPSEGS | |
| | APGTSESATPESGPGTSESATPESG | |
| 14 | WGQGTLVTVSGAPCGASPLGLSGRSDNHPGSPAGSPTSTEEGTSESATPESGPGTSTEP | 630 |
| | SEGSAPGSPAGSPTSTEEGTSTEPSEGSAPGTSTEPSEGSAPGTSESATPESGPGSEPA | |
| | TSGSETPGSEPATSGSETPGSPAGSPTSTEEGTSESATPESGPGTSTEPSEGSAPGTST | |
| | EPSEGSAPGSPAGSPTSTEEGTSTEPSEGSAPGTSTEPSEGSAPGTSESATPESGPGTS | |
| | TEPSEGSAPGTSESATPESGPGSEPATSGSETPGTSTEPSEGSAPGTSTEPSEGSAPGT | |
| | SESATPESGPGTSESATPESG | |
| 15 | WGQGTLVTVSGSPGAGSCAGSPTSTEEGTSESACSPEGPGTSTEPSEGSCGGSPLGLSG | 631 |
| | RSDNHPGSPAGSPTSTEEGTSESATPESGPGTSTEPSEGSAPGSPAGSPTSTEEGTSTE | |
| | PSEGSAPGTSTEPSEGSAPGTSESATPESGPGSEPATSGSETPGSEPATSGSETPGSPA | |
| | GSPTSTEEGTSESATPESGPGTSTEPSEGSAPGTSTEPSEGSAPGSPAGSPTSTEEGTS | |
| | TEPSEGSAPGTSTEPSEGSAPGTSESATPESGPGTSTEPSEGSAPGTSESATPESGPGS | |
| | EPATSGSETPGTSTEPSEGSAPGTSTEPSEGSAPGTSESATPESGPGTSESATPESG | |
| 16 | WGQGTLVTVSGAPCGAGCAGPCGPSPLGLSGRSDNHPGSPAGSPTSTEEGTSESATPES | 632 |
| | GPGTSTEPSEGSAPGSPAGSPTSTEEGTSTEPSEGSAPGTSTEPSEGSAPGTSESATPE | |
| | SGPGSEPATSGSETPGSEPATSGSETPGSPAGSPTSTEEGTSESATPESGPGTSTEPSE | |
| | GSAPGTSTEPSEGSAPGSPAGSPTSTEEGTSTEPSEGSAPGTSTEPSEGSAPGTSESAT | |
| | PESGPGTSTEPSEGSAPGTSESATPESGPGSEPATSGSETPGTSTEPSEGSAPGTSTEP | |
| | SEGSAPGTSESATPESGPGTSESATPESG | |
| 17 | WGQGTLVTVSGSPGAGSCAGSPTSTEEGTSESACSPEGPGTSTEPSEGSCGPSPAGSPT | 633 |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASGSPLGLSGRSDNHPGSPAGSPTST | |
| | EEGTSESATPESGPGTSTEPSEGSAPGSPAGSPTSTEEGTSTEPSEGSAPGTSTEPSEG | |
| | SAPGTSESATPESGPGSEPATSGSETPGSEPATSGSETPGSPAGSPTSTEEGTSESATP | |
| | ESGPGTSTEPSEGSAPGTSTEPSEGSAPGSPAGSPTSTEEGTSTEPSEGSAPGTSTEPS | |
| | EGSAPGTSESATPESGPGTSTEPSEGSAPGTSESATPESGPGSEPATSGSETPGTSTEP | |
| | SEGSAPGTSTEPSEGSAPGTSESATPESGPGTSESATPESG | |
| 18 | WGQGTLVTVSGSPGAGSCTETSPSTCPTESPEACGSGSGSPCSPSGTEACSTSGSEGCS | 634 |
| | PSSTAPCGPTETEGCTTSSGPPCPESATSEGSPLGLSGRSDNHPGSPAGSPTSTEEGTS | |
| | ESATPESGPGTSTEPSEGSAPGSPAGSPTSTEEGTSTEPSEGSAPGTSTEPSEGSAPGT | |
| | SESATPESGPGSEPATSGSETPGSEPATSGSETPGSPAGSPTSTEEGTSESATPESGPG | |
| | TSTEPSEGSAPGTSTEPSEGSAPGSPAGSPTSTEEGTSTEPSEGSAPGTSTEPSEGSAP | |
| | GTSESATPESGPGTSTEPSEGSAPGTSESATPESGPGSEPATSGSETPGTSTEPSEGSA | |
| | PGTSTEPSEGSAPGTSESATPESGPGTSESATPESG | |
| 19 | WGQGTLVTVSGSPGAGSCAGLAGRSDNHVPLSLSMGPGSPAGSPTSTEEGTSESATPES | 635 |
| | GPGTSTEPSEGSAPGSPAGSPTSTEEGTSTEPSEGSAPGTSTEPSEGSAPGTSESATPE | |
| | SGPGSEPATSGSETPGSEPATSGSETPGSPAGSPTSTEEGTSESATPESGPGTSTEPSE | |
| | GSAPGTSTEPSEGSAPGSPAGSPTSTEEGTSTEPSEGSAPGTSTEPSEGSAPGTSESAT | |
| | PESGPGTSTEPSEGSAPGTSESATPESGPGSEPATSGSETPGTSTEPSEGSAPGTSTEP | |
| | SEGSAPGTSESATPESGPGTSESATPESG | |
| 20 | WGQGTLVTVSGAPCGALAGRSDNHVPLSLSMGPGSPAGSPTSTEEGTSESATPESGPGT | 636 |
| | STEPSEGSAPGSPAGSPTSTEEGTSTEPSEGSAPGTSTEPSEGSAPGTSESATPESGPG | |
| | SEPATSGSETPGSEPATSGSETPGSPAGSPTSTEEGTSESATPESGPGTSTEPSEGSAP | |
| | GTSTEPSEGSAPGSPAGSPTSTEEGTSTEPSEGSAPGTSTEPSEGSAPGTSESATPESG | |
| | PGTSTEPSEGSAPGTSESATPESGPGSEPATSGSETPGTSTEPSEGSAPGTSTEPSEGS | |
| | APGTSESATPESGPGTSESATPESG | |
| 21 | WGQGTLVTVSGSPGAGSCAGSPTSTEEGTSESACSPEGPGTSTEPSEGSCGGLAGRSDN | 637 |
| | HVPLSLSMGPGSPAGSPTSTEEGTSESATPESGPGTSTEPSEGSAPGSPAGSPTSTEEG | |
| | TSTEPSEGSAPGTSTEPSEGSAPGTSESATPESGPGSEPATSGSETPGSEPATSGSETP | |
| | GSPAGSPTSTEEGTSESATPESGPGTSTEPSEGSAPGTSTEPSEGSAPGSPAGSPTSTE | |
| | EGTSTEPSEGSAPGTSTEPSEGSAPGTSESATPESGPGTSTEPSEGSAPGTSESATPES | |
| | GPGSEPATSGSETPGTSTEPSEGSAPGTSTEPSEGSAPGTSESATPESGPGTSESATPE | |
| | SG | |
| 22 | WGQGTLVTVSGAPCGAGCAGPCGPLAGRSDNHVPLSLSMGPGSPAGSPTSTEEGTSESA | 638 |
| | TPESGPGTSTEPSEGSAPGSPAGSPTSTEEGTSTEPSEGSAPGTSTEPSEGSAPGTSES | |
| | ATPESGPGSEPATSGSETPGSEPATSGSETPGSPAGSPTSTEEGTSESATPESGPGTST | |
| | EPSEGSAPGTSTEPSEGSAPGSPAGSPTSTEEGTSTEPSEGSAPGTSTEPSEGSAPGTS | |
| | ESATPESGPGTSTEPSEGSAPGTSESATPESGPGSEPATSGSETPGTSTEPSEGSAPGT | |
| | STEPSEGSAPGTSESATPESGPGTSESATPESG | |
| 23 | WGQGTLVTVSGSPGAGSCAGSPTSTEEGTSESACSPEGPGTSTEPSEGSCGPSPAGSPT | 639 |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASGLAGRSDNHVPLSLSMGPGSPAGS | |
| | PTSTEEGTSESATPESGPGTSTEPSEGSAPGSPAGSPTSTEEGTSTEPSEGSAPGTSTE | |
| | PSEGSAPGTSESATPESGPGSEPATSGSETPGSEPATSGSETPGSPAGSPTSTEEGTSE | |
| | SATPESGPGTSTEPSEGSAPGTSTEPSEGSAPGSPAGSPTSTEEGTSTEPSEGSAPGTS | |
| | TEPSEGSAPGTSESATPESGPGTSTEPSEGSAPGTSESATPESGPGSEPATSGSETPGT | |
| | STEPSEGSAPGTSTEPSEGSAPGTSESATPESGPGTSESATPESG | |
| 24 | WGQGTLVTVSGSPGAGSCTETSPSTCPTESPEACGSGSGSPCSPSGTEACSTSGSEGCS | 640 |
| | PSSTAPCGPTETEGCTTSSGPPCPESATSEGLAGRSDNHVPLSLSMGPGSPAGSPTSTE | |
| | EGTSESATPESGPGTSTEPSEGSAPGSPAGSPTSTEEGTSTEPSEGSAPGTSTEPSEGS | |
| | APGTSESATPESGPGSEPATSGSETPGSEPATSGSETPGSPAGSPTSTEEGTSESATPE | |
| | SGPGTSTEPSEGSAPGTSTEPSEGSAPGSPAGSPTSTEEGTSTEPSEGSAPGTSTEPSE | |
| | GSAPGTSESATPESGPGTSTEPSEGSAPGTSESATPESGPGSEPATSGSETPGTSTEPS | |
| | EGSAPGTSTEPSEGSAPGTSESATPESGPGTSESATPESG | |
| 25 | TFGQGTKVEIKGSPGAGSCAGLSGRSDNHSPLGLAGSPGSPAGSPTSTEEGTSESATPE | 641 |
| | SGPGTSTEPSEGSAPGSPAGSPTSTEEGTSTEPSEGSAPGTSTEPSEGSAPGTSESATP | |
| | ESGPGSEPATSGSETPGSEPATSGSETPGSPAGSPTSTEEGTSESATPESGPGTSTEPS | |
| | EGSAPGTSTEPSEGSAPGSPAGSPTSTEEGTSTEPSEGSAPGTSTEPSEGSAPGTSESA | |
| | TPESGPGTSTEPSEGSAPGTSESATPESGPGSEPATSGSETPGTSTEPSEGSAPGTSTE | |
| | PSEGSAPGTSESATPESGPGTSESATPESG | |
| 26 | TFGQGTKVEIKGAPCGALSGRSDNHSPLGLAGSPGSPAGSPTSTEEGTSESATPESGPG | 642 |
| | TSTEPSEGSAPGSPAGSPTSTEEGTSTEPSEGSAPGTSTEPSEGSAPGTSESATPESGP | |
| | GSEPATSGSETPGSEPATSGSETPGSPAGSPTSTEEGTSESATPESGPGTSTEPSEGSA | |
| | PGTSTEPSEGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPGTSTEPSEG | |
| | SAPGTSESATPESGPGTSESATESG | |
| 27 | TFGQGTKVEIKGSPGAGSCAGSPTSTEEGTSESACSPEGPGTSTEPSEGSCGGLSGRSD | 643 |
| | NHSPLGLAGSPGSPAGSPTSTEEGTSESATPESGPGTSTEPSEGSAPGSPAGSPTSTEE | |
| | GTSTEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSPTST | |
| | EEGTSTEPSEGSAPGTSTEPSEGSAPGTSESATPESGPGTSTEPSEGSAPGTSESATPE | |
| | SGPGSEPATSGSETPGTSTEPSEGSAPGTSTEPSEGSAPGTSESATPESGPGTSESATP | |
| | ESG | |
| 28 | TFGQGTKVEIKGAPCGAGCAGPCGPLSGRSDNHSPLGLAGSPGSPAGSPTSTEEGTSES | 644 |
| | ATPESGPGTSTEPSEGSAPGSPAGSPTSTEEGTSTEPSEGSAPGTSTEPSEGSAPGTSE | |
| | SATPESGPGSEPATSGSETPGSEPATSGSETPGSPAGSPTSTEEGTSESATPESGPGTS | |
| | TEPSEGSAPGTSTEPSEGSAPGSPAGSPTSTEEGTSTEPSEGSAPGTSTEPSEGSAPGT | |
| | SESATPESGPGTSTEPSEGSAPGTSESATPESGPGSEPATSGSETPGTSTEPSEGSAPG | |
| | TSTEPSEGSAPGTSESATPESGPGTSESATPESG | |
| 29 | TFGQGTKVEIKGSPGAGSCAGSPTSTEEGTSESACSPEGPGTSTEPSEGSCGPSPAGSP | 645 |
| | TSTEEGTCTEPSEGSAPGTSEPTCSGSAPGTSESATPESCGPSEPATSGSETPGSCAPT | |
| | SGSETPGSPAGSCTSTEEGTSESATPESCGPTESASGLSGRSDNHSPLGLAGSPGSPAG | |
| | SPTSTEEGTSESATPESGPGTSTEPSEGSAPGSPAGSPTSTEEGTSTEPSEGSAPGTST | |
| | EPSEGSAPGTSESATPESGPGSEPATSGSETPGSEPATSGSETPGSPAGSPTSTEEGTS | |
| | ESATPESGPGTSTEPSEGSAPGTSTEPSEGSAPGSPAGSPTSTEEGTSTEPSEGSAPGT | |
| | STEPSEGSAPGTSESATPESGPGTSTEPSEGSAPGTSESATPESGPGSEPATSGSETPG | |
| | TSTEPSEGSAPGTSTEPSEGSAPGTSESATPESGPGTSESATPESG | |
| 30 | TFGQGTKVEIKGSPGAGSCTETSPSTCPTESPEACGSGSGSPCSPSGTEACSTSGSEGC | 646 |
| | SPSSTAPCGPTETEGCTTSSGPPCPESATSEGLSGRSDNHSPLGLAGSPGSPAGSPTST | |
| | EEGTSESATPESGPGTSTEPSEGSAPGSPAGSPTSTEEGTSTEPSEGSAPGTSTEPSEG | |
| | SAPGTSESATPESGPGSEPATSGSETPGSEPATSGSETPGSPAGSPTSTEEGTSESATP | |
| | ESGPGTSTEPSEGSAPGTSTEPSEGSAPGSPAGSPTSTEEGTSTEPSEGSAPGTSTEPS | |
| | EGSAPGTSESATPESGPGTSTEPSEGSAPGTSESATPESGPGSEPATSGSETPGTSTEP | |
| | SEGSAPGTSTEPSEGSAPGTSESATPESGPGTSESATPESG | |
| 31 | TFGQGTKVEIKGSPGAGSCAGSPLGLAGSLSGRSDNHPGSPAGSPTSTEEGTSESATPE | 647 |
| | SGPGTSTEPSEGSAPGSPAGSPTSTEEGTSTEPSEGSAPGTSTEPSEGSAPGTSESATP | |
| | ESGPGSEPATSGSETPGSEPATSGSETPGSPAGSPTSTEEGTSESATPESGPGTSTEPS | |
| | EGSAPGTSTEPSEGSAPGSPAGSPTSTEEGTSTEPSEGSAPGTSTEPSEGSAPGTSESA | |
| | TPESGPGTSTEPSEGSAPGTSESATPESGPGSEPATSGSETPGTSTEPSEGSAPGTSTE | |
| | PSEGSAPGTSESATPESGPGTSESATPESG | |
| 32 | TFGQGTKVEIKGAPCGASPLGLAGSLSGRSDNHPGSPAGSPTSTEEGTSESATPESGPG | 648 |
| | TSTEPSEGSAPGSPAGSPTSTEEGTSTEPSEGSAPGTSTEPSEGSAPGTSESATPESGP | |
| | GSEPATSGSETPGSEPATSGSETPGSPAGSPTSTEEGTSESATPESGPGTSTEPSEGSA | |
| | PGTSTEPSEGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPGTSTEPSEG | |
| | SAPGTSESATPESGPGTSESATPESG | |
| 33 | TFGQGTKVEIKGSPGAGSCAGSPTSTEEGTSESACSPEGPGTSTEPSEGSCGGSPLGLA | 649 |
| | GSLSGRSDNHPGSPAGSPTSTEEGTSESATPESGPGTSTEPSEGSAPGSPAGSPTSTEE | |
| | GTSTEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSPTST | |
| | EEGTSTEPSEGSAPGTSTEPSEGSAPGTSESATPESGPGTSTEPSEGSAPGTSESATPE | |
| | SGPGSEPATSGSETPGTSTEPSEGSAPGTSTEPSEGSAPGTSESATPESGPGTSESATP | |
| | ESG | |
| 34 | TFGQGTKVEIKGAPCGAGCAGPCGPSPLGLAGSLSGRSDNHPGSPAGSPTSTEEGTSES | 650 |
| | ATPESGPGTSTEPSEGSAPGSPAGSPTSTEEGTSTEPSEGSAPGTSTEPSEGSAPGTSE | |
| | SATPESGPGSEPATSGSETPGSEPATSGSETPGSPAGSPTSTEEGTSESATPESGPGTS | |
| | TEPSEGSAPGTSTEPSEGSAPGSPAGSPTSTEEGTSTEPSEGSAPGTSTEPSEGSAPGT | |
| | SESATPESGPGTSTEPSEGSAPGTSESATPESGPGSEPATSGSETPGTSTEPSEGSAPG | |
| | TSTEPSEGSAPGTSESATPESGPGTSESATPESG | |
| 35 | TFGQGTKVEIKGSPGAGSCAGSPTSTEEGTSESACSPEGPGTSTEPSEGSCGPSPAGSP | 651 |
| | TSTEEGTCTEPSEGSAPGTSEPTCSGSAPGTSESATPESCGPSEPATSGSETPGSCAPT | |
| | SGSETPGSPAGSCTSTEEGTSESATPESCGPTESASGSPLGLAGSLSGRSDNHPGSPAG | |
| | SPTSTEEGTSESATPESGPGTSTEPSEGSAPGSPAGSPTSTEEGTSTEPSEGSAPGTST | |
| | EPSEGSAPGTSESATPESGPGSEPATSGSETPGSEPATSGSETPGSPAGSPTSTEEGTS | |
| | ESATPESGPGTSTEPSEGSAPGTSTEPSEGSAPGSPAGSPTSTEEGTSTEPSEGSAPGT | |
| | STEPSEGSAPGTSESATPESGPGTSTEPSEGSAPGTSESATPESGPGSEPATSGSETPG | |
| | TSTEPSEGSAPGTSTEPSEGSAPGTSESATPESGPGTSESATPESG | |
| 36 | TFGQGTKVEIKGSPGAGSCTETSPSTCPTESPEACGSGSGSPCSPSGTEACSTSGSEGC | 652 |
| | SPSSTAPCGPTETEGCTTSSGPPCPESATSEGSPLGLAGSLSGRSDNHPGSPAGSPTST | |
| | EEGTSESATPESGPGTSTEPSEGSAPGSPAGSPTSTEEGTSTEPSEGSAPGTSTEPSEG | |
| | SAPGTSESATPESGPGSEPATSGSETPGSEPATSGSETPGSPAGSPTSTEEGTSESATP | |
| | ESGPGTSTEPSEGSAPGTSTEPSEGSAPGSPAGSPTSTEEGTSTEPSEGSAPGTSTEPS | |
| | EGSAPGTSESATPESGPGTSTEPSEGSAPGTSESATPESGPGSEPATSGSETPGTSTEP | |
| | SEGSAPGTSTEPSEGSAPGTSESATPESGPGTSESATPESG | |
| 37 | TFGQGTKVEIKGSPGAGSCAGSPLGLSGRSDNHPGSPAGSPTSTEEGTSESATPESGPG | 653 |
| | TSTEPSEGSAPGSPAGSPTSTEEGTSTEPSEGSAPGTSTEPSEGSAPGTSESATPESGP | |
| | GSEPATSGSETPGSEPATSGSETPGSPAGSPTSTEEGTSESATPESGPGTSTEPSEGSA | |
| | PGTSTEPSEGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPGTSTEPSEG | |
| | SAPGTSESATPESGPGTSESATPESG | |
| 38 | TFGQGTKVEIKGAPCGASPLGLSGRSDNHPGSPAGSPTSTEEGTSESATPESGPGTSTE | 654 |
| | PSEGSAPGSPAGSPTSTEEGTSTEPSEGSAPGTSTEPSEGSAPGTSESATPESGPGSEP | |
| | ATSGSETPGSEPATSGSETPGSPAGSPTSTEEGTSESATPESGPGTSTEPSEGSAPGTS | |
| | TEPSEGSAPGSPAGSPTSTEEGTSTEPSEGSAPGTSTEPSEGSAPGTSESATPESGPGT | |
| | STEPSEGSAPGTSESATPESGPGSEPATSGSETPGTSTEPSEGSAPGTSTEPSEGSAPG | |
| | TSESATPESGPGTSESATPESG | |
| 39 | TFGQGTKVEIKGSPGAGSCAGSPTSTEEGTSESACSPEGPGTSTEPSEGSCGGSPLGLS | 655 |
| | GRSDNHPGSPAGSPTSTEEGTSESATPESGPGTSTEPSEGSAPGSPAGSPTSTEEGTST | |
| | EPSEGSAPGTSTEPSEGSAPGTSESATPESGPGSEPATSGSETPGSEPATSGSETPGSP | |
| | AGSPTSTEEGTSESATPESGPGTSTEPSEGSAPGTSTEPSEGSAPGSPAGSPTSTEEGT | |
| | STEPSEGSAPGTSTEPSEGSAPGTSESATPESGPGTSTEPSEGSAPGTSESATPESGPG | |
| | SEPATSGSETPGTSTEPSEGSAPGTSTEPSEGSAPGTSESATPESGPGTSESATPESG | |
| 40 | TFGQGTKVEIKGAPCGAGCAGPCGPSPLGLSGRSDNHPGSPAGSPTSTEEGTSESATPE | 656 |
| | SGPGTSTEPSEGSAPGSPAGSPTSTEEGTSTEPSEGSAPGTSTEPSEGSAPGTSESATP | |
| | ESGPGSEPATSGSETPGSEPATSGSETPGSPAGSPTSTEEGTSESATPESGPGTSTEPS | |
| | EGSAPGTSTEPSEGSAPGSPAGSPTSTEEGTSTEPSEGSAPGTSTEPSEGSAPGTSESA | |
| | TPESGPGTSTEPSEGSAPGTSESATPESGPGSEPATSGSETPGTSTEPSEGSAPGTSTE | |
| | PSEGSAPGTSESATPESGPGTSESATPESG | |
| 41 | TFGQGTKVEIKGSPGAGSCAGSPTSTEEGTSESACSPEGPGTSTEPSEGSCGPSPAGSP | 657 |
| | TSTEEGTCTEPSEGSAPGTSEPTCSGSAPGTSESATPESCGPSEPATSGSETPGSCAPT | |
| | SGSETPGSPAGSCTSTEEGTSESATPESCGPTESASGSPLGLSGRSDNHPGSPAGSPTS | |
| | TEEGTSESATPESGPGTSTEPSEGSAPGSPAGSPTSTEEGTSTEPSEGSAPGTSTEPSE | |
| | GSAPGTSESATPESGPGSEPATSGSETPGSEPATSGSETPGSPAGSPTSTEEGTSESAT | |
| | PESGPGTSTEPSEGSAPGTSTEPSEGSAPGSPAGSPTSTEEGTSTEPSEGSAPGTSTEP | |
| | SEGSAPGTSESATPESGPGTSTEPSEGSAPGTSESATPESGPGSEPATSGSETPGTSTE | |
| | PSEGSAPGTSTEPSEGSAPGTSESATPESGPGTSESATPESG | |
| 42 | TFGQGTKVEIKGSPGAGSCTETSPSTCPTESPEACGSGSGSPCSPSGTEACSTSGSEGC | 658 |
| | SPSSTAPCGPTETEGCTTSSGPPCPESATSEGSPLGLSGRSDNHPGSPAGSPTSTEEGT | |
| | SESATPESGPGTSTEPSEGSAPGSPAGSPTSTEEGTSTEPSEGSAPGTSTEPSEGSAPG | |
| | TSESATPESGPGSEPATSGSETPGSEPATSGSETPGSPAGSPTSTEEGTSESATPESGP | |
| | GTSTEPSEGSAPGTSTEPSEGSAPGSPAGSPTSTEEGTSTEPSEGSAPGTSTEPSEGSA | |
| | PGTSESATPESGPGTSTEPSEGSAPGTSESATPESGPGSEPATSGSETPGTSTEPSEGS | |
| | APGTSTEPSEGSAPGTSESATPESGPGTSESATPESG | |
| 43 | TFGQGTKVEIKGSPGAGSCAGLAGRSDNHVPLSLSMGPGSPAGSPTSTEEGTSESATPE | 659 |
| | SGPGTSTEPSEGSAPGSPAGSPTSTEEGTSTEPSEGSAPGTSTEPSEGSAPGTSESATP | |
| | ESGPGSEPATSGSETPGSEPATSGSETPGSPAGSPTSTEEGTSESATPESGPGTSTEPS | |
| | EGSAPGTSTEPSEGSAPGSPAGSPTSTEEGTSTEPSEGSAPGTSTEPSEGSAPGTSESA | |
| | TPESGPGTSTEPSEGSAPGTSESATPESGPGSEPATSGSETPGTSTEPSEGSAPGTSTE | |
| | PSEGSAPGTSESATPESGPGTSESATPESG | |
| 44 | TFGQGTKVEIKGAPCGALAGRSDNHVPLSLSMGPGSPAGSPTSTEEGTSESATPESGPG | 660 |
| | TSTEPSEGSAPGSPAGSPTSTEEGTSTEPSEGSAPGTSTEPSEGSAPGTSESATPESGP | |
| | GSEPATSGSETPGSEPATSGSETPGSPAGSPTSTEEGTSESATPESGPGTSTEPSEGSA | |
| | PGTSTEPSEGSAPGSPAGSPTSTEEGTSTEPSEGSAPGTSTEPSEGSAPGTSESATPES | |
| | GPGTSTEPSEGSAPGTSESATPESGPGSEPATSGSETPGTSTEPSEGSAPGTSTEPSEG | |
| | SAPGTSESATPESGPGTSESATPESG | |
| 45 | TFGQGTKVEIKGSPGAGSCAGSPTSTEEGTSESACSPEGPGTSTEPSEGSCGGLAGRSD | 661 |
| | NHVPLSLSMGPGSPAGSPTSTEEGTSESATPESGPGTSTEPSEGSAPGSPAGSPTSTEE | |
| | GTSTEPSEGSAPGTSTEPSEGSAPGTSESATPESGPGSEPATSGSETPGSEPATSGSET | |
| | PGSPAGSPTSTEEGTSESATPESGPGTSTEPSEGSAPGTSTEPSEGSAPGSPAGSPTST | |
| | EEGTSTEPSEGSAPGTSTEPSEGSAPGTSESATPESGPGTSTEPSEGSAPGTSESATPE | |
| | SGPGSEPATSGSETPGTSTEPSEGSAPGTSTEPSEGSAPGTSESATPESGPGTSESATP | |
| | ESG | |
| 46 | TFGQGTKVEIKGAPCGAGCAGPCGPLAGRSDNHVPLSLSMGPGSPAGSPTSTEEGTSES | 662 |
| | ATPESGPGTSTEPSEGSAPGSPAGSPTSTEEGTSTEPSEGSAPGTSTEPSEGSAPGTSE | |
| | SATPESGPGSEPATSGSETPGSEPATSGSETPGSPAGSPTSTEEGTSESATPESGPGTS | |
| | TEPSEGSAPGTSTEPSEGSAPGSPAGSPTSTEEGTSTEPSEGSAPGTSTEPSEGSAPGT | |
| | SESATPESGPGTSTEPSEGSAPGTSESATPESGPGSEPATSGSETPGTSTEPSEGSAPG | |
| | TSTEPSEGSAPGTSESATPESGPGTSESATPESG | |
| 47 | TFGQGTKVEIKGSPGAGSCAGSPTSTEEGTSESACSPEGPGTSTEPSEGSCGPSPAGSP | 663 |
| | TSTEEGTCTEPSEGSAPGTSEPTCSGSAPGTSESATPESCGPSEPATSGSETPGSCAPT | |
| | SGSETPGSPAGSCTSTEEGTSESATPESCGPTESASGLAGRSDNHVPLSLSMGPGSPAG | |
| | SPTSTEEGTSESATPESGPGTSTEPSEGSAPGSPAGSPTSTEEGTSTEPSEGSAPGTST | |
| | EPSEGSAPGTSESATPESGPGSEPATSGSETPGSEPATSGSETPGSPAGSPTSTEEGTS | |
| | ESATPESGPGTSTEPSEGSAPGTSTEPSEGSAPGSPAGSPTSTEEGTSTEPSEGSAPGT | |
| | STEPSEGSAPGTSESATPESGPGTSTEPSEGSAPGTSESATPESGPGSEPATSGSETPG | |
| | TSTEPSEGSAPGTSTEPSEGSAPGTSESATPESGPGTSESATPESG | |
| 48 | TEGQGTKVEIKGSPGAGSCTETSPSTCPTESPEACGSGSGSPCSPSGTEACSTSGSEGC | 664 |
| | SPSSTAPCGPTETEGCTTSSGPPCPESATSEGLAGRSDNHVPLSLSMGPGSPAGSPTST | |
| | EEGTSESATPESGPGTSTEPSEGSAPGSPAGSPTSTEEGTSTEPSEGSAPGTSTEPSEG | |
| | SAPGTSESATPESGPGSEPATSGSETPGSEPATSGSETPGSPAGSPTSTEEGTSESATP | |
| | ESGPGTSTEPSEGSAPGTSTEPSEGSAPGSPAGSPTSTEEGTSTEPSEGSAPGTSTEPS | |
| | EGSAPGTSESATPESGPGTSTEPSEGSAPGTSESATPESGPGSEPATSGSETPGTSTEP | |
| | SEGSAPGTSTEPSEGSAPGTSESATPESGPGTSESATPESG | |

*Sequence is the N- to C-terminus sequence of fusion of FR4-CCD-PCM-XTEN

TABLE 27
Concatenates of fusion protein components for targeted coniugate compositions

| | | SEQ ID |
|---|---|---|
| No. | Amino Acid Sequences of XTEN-PCM-CCD-FI21* | NO: |
| 1 | GTSESATPESGPGSEPATSGSETPGTSESATPESGPGSEPATSGSETPGTSESATPESG | 665 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGEVQLVE | |
| | SGGGLSGRSDNHSPLGLAGSGSPGAGSCAG | |
| 2 | GTSESATPESGPGSEPATSGSETPGTSESATPESGPGSEPATSGSETPGTSESATPESG | 666 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGDIQMTQ | |
| | SPSSSPLGLAGSLSGRSDNHGSPGAGSCAG | |
| 3 | GTSESATPESGPGSEPATSGSETPGTSESATPESGPGSEPATSGSETPGTSESATPESG | 667 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAG | |
| 4 | GTSESATPESGPGSEPATSGSETPGTSESATPESGPGSEPATSGSETPGTSESATPESG | 668 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAG | |
| 5 | GTSESATPESGPGSEPATSGSETPGTSESATPESGPGSEPATSGSETPGTSESATPESG | 669 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGAPCGA | |
| 6 | GTSESATPESGPGSEPATSGSETPGTSESATPESGPGSEPATSGSETPGTSESATPESG | 670 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGAPCGA | |
| 7 | GTSESATPESGPGSEPATSGSETPGTSESATPESGPGSEPATSGSETPGTSESATPESG | 671 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGAPCGA | |
| 8 | GTSESATPESGPGSEPATSGSETPGTSESATPESGPGSEPATSGSETPGTSESATPESG | 672 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGAPCGA | |
| 9 | GTSESATPESGPGSEPATSGSETPGTSESATPESGPGSEPATSGSETPGTSESATPESG | 673 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCAGSPTSTEEGTSESACSPEGPGTSTEPSEGSCGG | |
| 10 | GTSESATPESGPGSEPATSGSETPGTSESATPESGPGSEPATSGSETPGTSESATPESG | 674 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCAGSPTSTEEGTSESACSPEGPGTSTEPSEGSCGG | |
| 11 | GTSESATPESGPGSEPATSGSETPGTSESATPESGPGSEPATSGSETPGTSESATPESG | 675 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAGSPTSTEEGTSESACSPEGPGTSTEPSEGSCGG | |
| 12 | GTSESATPESGPGSEPATSGSETPGTSESATPESGPGSEPATSGSETPGTSESATPESG | 676 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAGSPTSTEEGTSESACSPEGPGTSTEPSEGSCGG | |
| 13 | GTSESATPESGPGSEPATSGSETPGTSESATPESGPGSEPATSGSETPGTSESATPESG | 677 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGAPCGAGCAGPCGP | |
| 14 | GTSESATPESGPGSEPATSGSETPGTSESATPESGPGSEPATSGSETPGTSESATPESG | 678 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGAPCGAGCAGPCGP | |
| 15 | GTSESATPESGPGSEPATSGSETPGTSESATPESGPGSEPATSGSETPGTSESATPESG | 679 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGAPCGAGCAGPCGP | |
| 16 | GTSESATPESGPGSEPATSGSETPGTSESATPESGPGSEPATSGSETPGTSESATPESG | 680 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGAPCGAGCAGPCGP | |
| 17 | GTSESATPESGPGSEPATSGSETPGTSESATPESGPGSEPATSGSETPGTSESATPESG | 681 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 18 | GTSESATPESGPGSEPATSGSETPGTSESATPESGPGSEPATSGSETPGTSESATPESG | 682 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 19 | GTSESATPESGPGSEPATSGSETPGTSESATPESGPGSEPATSGSETPGTSESATPESG | 683 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAGSPTSTEEGTSESACSPEGPGTSTEPSEGSCGPSPAGSPTSTEE | |
| | GTCTEPSEGSAPGTSEPTCSGSAPGTSESATPESCGPSEPATSGSETPGSCAPTSGSET | |
| | PGSPAGSCTSTEEGTSESATPESCGPTESASG | |
| 20 | GTSESATPESGPGSEPATSGSETPGTSESATPESGPGSEPATSGSETPGTSESATPESG | 684 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 21 | GTSESATPESGPGSEPATSGSETPGTSESATPESGPGSEPATSGSETPGTSESATPESG | 685 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCTETSPSTCPTESPEACGSGSGSPCSPSGTEACSTSGSEGCS | |
| | PSSTAPCGPTETEGCTTSSGPPCPESATSEG | |
| 22 | GTSESATPESGPGSEPATSGSETPGTSESATPESGPGSEPATSGSETPGTSESATPESG | 686 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCTETSPSTCPTESPEACGSGSGSPCSPSGTEACSTSGSEGCS | |
| | PSSTAPCGPTETEGCTTSSGPPCPESATSEG | |
| 23 | GTSESATPESGPGSEPATSGSETPGTSESATPESGPGSEPATSGSETPGTSESATPESG | 687 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCTETSPSTCPTESPEACGSGSGSPCSPSGTEACSTSGSEGCSPSST | |
| | APCGPTETEGCTTSSGPPCPESATSEG | |
| 24 | GTSESATPESGPGSEPATSGSETPGTSESATPESGPGSEPATSGSETPGTSESATPESG | 688 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCTETSPSTCPTESPEACGSGSGSPCSPSGTEACSTSGSEGCS | |
| | PSSTAPCGPTETEGCTTSSGPPCPESATSEG | |
| 25 | GTSESATPESGPGSEPATSGSETPGTSESATPESGPGSEPATSGSETPGTSESATPESG | 689 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCAG | |
| 26 | GTSESATPESGPGSEPATSGSETPGTSESATPESGPGSEPATSGSETPGTSESATPESG | 690 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCAG | |
| 27 | GTSESATPESGPGSEPATSGSETPGTSESATPESGPGSEPATSGSETPGTSESATPESG | 691 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAG | |
| 28 | GTSESATPESGPGSEPATSGSETPGTSESATPESGPGSEPATSGSETPGTSESATPESG | 692 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAG | |
| 29 | GTSESATPESGPGSEPATSGSETPGTSESATPESGPGSEPATSGSETPGTSESATPESG | 693 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGAPCGA | |
| 30 | GTSESATPESGPGSEPATSGSETPGTSESATPESGPGSEPATSGSETPGTSESATPESG | 694 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGAPCGA | |
| 31 | GTSESATPESGPGSEPATSGSETPGTSESATPESGPGSEPATSGSETPGTSESATPESG | 695 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGAPCGA | |
| 32 | GTSESATPESGPGSEPATSGSETPGTSESATPESGPGSEPATSGSETPGTSESATPESG | 696 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGAPCGA | |
| 33 | GTSESATPESGPGSEPATSGSETPGTSESATPESGPGSEPATSGSETPGTSESATPESG | 697 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCAGSPTSTEEGTSESACSPEGPGTSTEPSEGSCGG | |
| 34 | GTSESATPESGPGSEPATSGSETPGTSESATPESGPGSEPATSGSETPGTSESATPESG | 698 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCAGSPTSTEEGTSESACSPEGPGTSTEPSEGSCGG | |
| 35 | GTSESATPESGPGSEPATSGSETPGTSESATPESGPGSEPATSGSETPGTSESATPESG | 699 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAGSPTSTEEGTSESACSPEGPGTSTEPSEGSCGG | |
| 36 | GTSESATPESGPGSEPATSGSETPGTSESATPESGPGSEPATSGSETPGTSESATPESG | 700 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAGSPTSTEEGTSESACSPEGPGTSTEPSEGSCGG | |
| 37 | GTSESATPESGPGSEPATSGSETPGTSESATPESGPGSEPATSGSETPGTSESATPESG | 701 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGAPCGAGCAGPCGP | |
| 38 | GTSESATPESGPGSEPATSGSETPGTSESATPESGPGSEPATSGSETPGTSESATPESG | 702 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGAPCGAGCAGPCGP | |
| 39 | GTSESATPESGPGSEPATSGSETPGTSESATPESGPGSEPATSGSETPGTSESATPESG | 703 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGAPCGAGCAGPCGP | |
| 40 | GTSESATPESGPGSEPATSGSETPGTSESATPESGPGSEPATSGSETPGTSESATPESG | 704 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGAPCGAGCAGPCGP | |
| 41 | GTSESATPESGPGSEPATSGSETPGTSESATPESGPGSEPATSGSETPGTSESATPESG | 705 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 42 | GTSESATPESGPGSEPATSGSETPGTSESATPESGPGSEPATSGSETPGTSESATPESG | 706 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 43 | GTSESATPESGPGSEPATSGSETPGTSESATPESGPGSEPATSGSETPGTSESATPESG | 707 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCAGSPTSTEEGTSESACSPEGPGTSTEPSEGSCGPSPAGSPTSTEE | |
| | GTCTEPSEGSAPGTSEPTCSGSAPGTSESATPESCGPSEPATSGSETPGSCAPTSGSET | |
| | PGSPAGSCTSTEEGTSESATPESCGPTESASG | |
| 44 | GTSESATPESGPGSEPATSGSETPGTSESATPESGPGSEPATSGSETPGTSESATPESG | 708 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCAGSPTSTEEGTSESACSPEGPGTSTEPSEGSCGPSPAGSPT | |
| | STEEGTCTEPSEGSAPGTSEPTCSGSAPGTSESATPESCGPSEPATSGSETPGSCAPTS | |
| | GSETPGSPAGSCTSTEEGTSESATPESCGPTESASG | |
| 45 | GTSESATPESGPGSEPATSGSETPGTSESATPESGPGSEPATSGSETPGTSESATPESG | 709 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLSGRSD | |
| | NHSPLGLAGSGSPGAGSCTETSPSTCPTESPEACGSGSGSPCSPSGTEACSTSGSEGCS | |
| | PSSTAPCGPTETEGCTTSSGPPCPESATSEG | |
| 46 | GTSESATPESGPGSEPATSGSETPGTSESATPESGPGSEPATSGSETPGTSESATPESG | 710 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLA | |
| | GSLSGRSDNHGSPGAGSCTETSPSTCPTESPEACGSGSGSPCSPSGTEACSTSGSEGCS | |
| | PSSTAPCGPTETEGCTTSSGPPCPESATSEG | |
| 47 | GTSESATPESGPGSEPATSGSETPGTSESATPESGPGSEPATSGSETPGTSESATPESG | 711 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGSPLGLS | |
| | GRSDNHGSPGAGSCTETSPSTCPTESPEACGSGSGSPCSPSGTEACSTSGSEGCSPSST | |
| | APCGPTETEGCTTSSGPPCPESATSEG | |
| 48 | GTSESATPESGPGSEPATSGSETPGTSESATPESGPGSEPATSGSETPGTSESATPESG | 712 |
| | PGTSTEPSEGSAPGSPAGSPTSTEEGTSESATPESGPGSEPATSGSETPGTSESATPES | |
| | GPGSPAGSPTSTEEGSPAGSPTSTEEGTSTEPSEGSAPGTSESATPESGPGTSESATPE | |
| | SGPGTSESATPESGPGSEPATSGSETPGSEPATSGSETPGSPAGSPTSTEEGTSTEPSE | |
| | GSAPGTSTEPSEGSAPGSEPATSGSETPGTSESATPESGPGTSTEPSEGSAPGLAGRSD | |
| | NHVPLSLSMGGSPGAGSCTETSPSTCPTESPEACGSGSGSPCSPSGTEACSTSGSEGCS | |
| | PSSTAPCGPTETEGCTTSSGPPCPESATSEG | |

*Sequence is the N- to C-terminus sequence of fusion of XTEN-PCM-CCD-FR1*

The following are examples of compositions, methods, and treatment regimens of the invention. It is understood that various other embodiments may be practiced, given the general description provided above.

### EXAMPLES

### Example 1

### Construction of XTEN_AE864

XTEN_AE864 was constructed from serial dimerization of XTEN_AE36 to AE72, 144, 288, 576 and 864. A collection of XTEN_AE72 segments was constructed from 37 different segments of XTEN_AE36. Cultures of E. coli harboring all 37 different 36-amino acid segments were mixed and plasmid was isolated. This plasmid pool was digested with BsaI/NcoI to generate the small fragment as the insert. The same plasmid pool was digested with BbsI/NcoI to generate the large fragment as the vector. The insert and vector fragments were ligated resulting in a doubling of the length and the ligation mixture was transformed into BL21Gold(DE3) cells to obtain colonies of XTEN_AE72.

This library of XTEN_AE72 segments was designated LCW0406. All clones from LCW0406 were combined and dimerized again using the same process as described above yielding library LCW0410 of XTEN_AE144. All clones from LCW0410 were combined and dimerized again using the same process as described above yielding library LCW0414 of XTEN_AE288. Two isolates LCW0414.001 and LCW0414.002 were randomly picked from the library and sequenced to verify the identities. All clones from LCW0414 were combined and dimerized again using the same process as described above yielding library LCW0418 of XTEN_AE576. We screened 96 isolates from library LCW0418 for high level of GFP fluorescence. 8 isolates with right sizes of inserts by PCR and strong fluorescence were sequenced and 2 isolates (LCW0418.018 and LCW0418.052) were chosen for future use based on sequencing and expression data.

The specific clone pCW0432 of XTEN_AE864 was constructed by combining LCW0418.018 of XTEN_AE576 and LCW0414.002 of XTEN_AE288 using the same dimerization process as described above.

### Example 2

### Construction of XTEN_AG864

Using the several consecutive rounds of dimerization, we assembled a collection of XTEN_AG864 sequences starting from segments of XTEN_AD36. The methods and materials utilized were adapted from those of Example 1, above. These sequences were assembled and several isolates from XTEN_AG864 were evaluated and found to show good expression and excellent solubility under physiological conditions. A full length clone of XTEN_AG864 had excellent solubility and showed half-life exceeding 60 h in cynomolgus monkeys.

### Example 3

### Construction of CXTEN (1xAmino, 3xThiol-XTEN864)

PCR reaction was performed on plasmid pYS0072 with pairs of primers T7 promoter& SASRSABsaIrev-AACG (“SASRSA” disclosed as SEQ ID NO: 218), CH-AE864BsaIfor-AACG&AE432-Cl2BbsIrev2 and Cl2-AE432BsaIfor2&AE_003SbfIrev to obtain the PCR products of CI1, CI2 and AE-SbfI. Gel-purify the bands of right sizes and digest with the corresponding suitable restriction enzymes as the inserts.

Digest plasmid pYS0072 with NdeI/SbfI and gel-purify the large fragment as the vector. Ligate the vector with inserts above and transform into DH5α competent cells to screen by sequencing confirmation for the right clone of pCW1305, which encodes the protein of HD2-R-XTEN_AE864 (C12, C432, C854)-R-H8. The DNA sequences and protein sequences are provided in the Table 28, below.

TABLE 28
DNA and amino acid sequence for CXTEN (1xAmino, 3-Thio-XTEN864)

| Clone | | |
|---|---|---|
| Name | DNA Sequence | Amino Acid Sequence |
| N-term- | ATGAAAAACCCAGAGCAAGCAGAAGAACAAGCTGA | MKNPEQAEEQAEEQR |
| HD2-R- | AGAACAGCGCGAAGAAACAagcgcgtctcgttccgctGGGTC | EETSASRSAGSPTAEA |
| AE864_ | TCCAACGGCAGAAGCCGCTGGCtgcGGTACTGCTGAA | AGCGTAEAATSESATP |
| 3Cys-R- | GCGGCAACCTCTGAATCCGCTACTCCAGAATCCGGT | ESGPGTSTEPSEGSAP |
| H8 | CCTGGTACTAGCACTGAGCCAAGCGAAGGTTCTGCT | GSPAGSPTSTEEGTST |
| | CCAGGCTCCCCGGCAGGTAGCCCTACCTCTACCGAA | EPSEGSAPGTSTEPSEG |
| | GAGGGCACTAGCACCGAACCATCTGAGGGTTCCGCT | SAPGTSESATPESGPG |
| | CCTGGCACCTCCACTGAACCGTCCGAAGGCAGTGCT | SEPATSGSETPGSEPAT |
| | CCGGGTACTTCCGAAAGCGCAACTCCGGAATCCGGC | SGSETPGSPAGSPTSTE |
| | CCTGGTTCTGAGCCTGCTACTTCCGGCTCTGAAACTC | EGTSESATPESGPGTS |
| | CAGGTAGCGAGCCAGCGACTTCTGGTTCTGAAACTC | TEPSEGSAPGTSTEPSE |
| | CAGGTTCACCGGCGGGTAGCCCGACGAGCACGGAG | GSAPGSPAGSPTSTEE |
| | GAAGGTACCTCTGAGTCGGCCACTCCTGAGTCCGGT | GTSTEPSEGSAPGTST |
| | CCGGGCACGAGCACCGAGCCGAGCGAGGGTTCAGC | EPSEGSAPGTSESATPE |
| | CCCGGGTACCAGCACGGAGCCGTCCGAGGGTAGCG | SGPGTSTEPSEGSAPG |
| | CACCGGGTTCTCCGGCGGGCTCCCCTACGTCTACGG | TSESATPESGPGSEPAT |
| | AAGAGGGTACGTCCACTGAACCTAGCGAGGGCAGC | SGSETPGTSTEPSEGSA |
| | GCGCCAGGCACCAGCACTGAACCGAGCGAAGGCAG | PGTSTEPSEGSAPGTSE |
| | CGCACCTGGCACTAGCGAGTCTGCGACTCCGGAGAG | SATPESGPGTSESATPE |
| | CGGTCCGGGTACGAGCACGGAACCAAGCGAAGGCA | SGPGSPAGSPTSTEEG |
| | GCGCCCCAGGTACCTCTGAATCTGCTACCCCAGAAT | TSESATPESGPGSEPAT |
| | CTGGCCCGGGTTCCGAGCCAGCTACCTCTGGTTCTG | SGSETPGTSESATPESG |
| | AAACCCCAGGTACTTCCACTGAACCAAGCGAAGGT | PGTSTEPSEGSAPGTST |
| | AGCGCTCCTGGCACTTCTACTGAACCATCCGAAGGT | EPSEGSAPGTSTEPSEG |
| | TCCGCTCCTGGTACGTCTGAAAGCGCTACCCCTGAA | SAPGTSTEPSEGSAPG |
| | AGCGGCCCAGGCACCTCTGAAAGCGCTACTCCTGAG | TSTEPSEGSAPGTSTEP |
| | AGCGGTCCAGGCTCTCCAGCAGGTTCTCCAACCTCC | SEGSAPGSPAGSPTST |
| | ACTGAAGAAGGCACCTCTGAGTCTGCTACCCCTGAA | EETAEAAGCGTAEAA |
| | TCTGGTCCTGGCTCCGAACCTGCTACCTCTGGTTCCG | TSESATPESGPGSEPAT |
| | AAACTCCAGGTACCTCGGAATCTGCGACTCCGGAAT | SGSETPGTSESATPESG |
| | CTGGCCCGGGCACGAGCACGGAGCCGTCTGAGGGT | PGSEPATSGSETPGTSE |
| | AGCGCACCAGGTACCAGCACTGAGCCTTCTGAGGGC | SATPESGPGTSTEPSEG |
| | TCTGCACCGGGTACCTCCACGGAACCTTCGGAAGGT | SAPGTSESATPESGPG |
| | TCTGCGCCGGGTACCTCCACTGAGCCATCCGAGGGT | SPAGSPTSTEEGSPAG |
| | TCAGCACCAGGTACTAGCACGGAACCGTCCGAGGG | SPTSTEEGSPAGSPTST |
| | CTCTGCACCAGGTACGAGCACCGAACCGTCGGAGG | EEGTSESATPESGPGT |
| | GTAGCGCTCCAGGTAGCCCAGCGGGCTCTCCGACAA | STEPSEGSAPGTSESAT |
| | GCACCGAAGAAACCGCTGAAGCCGCAGGTtgtGGCAC | PESGPGSEPATSGSETP |
| | TGCGGAAGCTGCAACAAGCGAGAGCGCGACTCCTG | GTSESATPESGPGSEP |
| | AATCTGGTCCGGGTAGCGAGCCTGCAACCAGCGGTT | ATSGSETPGTSESATP |
| | CTGAGACGCCGGGCACTTCCGAATCTGCGACCCCGG | ESGPGTSTEPSEGSAP |
| | AGTCCGGTCCAGGTTCAGAGCCGGCGACGAGCGGTT | GSPAGSPTSTEEGTSES |
| | CGGAAACGCCGGGTACGTCTGAATCAGCCACGCCG | ATPESGPGSEPATSGS |
| | GAGTCTGGTCCGGGTACCTCGACCGAACCAAGCGA | ETPGTSESATPESGPGS |
| | AGGTTCGGCACCGGGTACTAGCGAGAGCGCAACCC | PAGSPTSTEEGSPAGS |
| | CTGAAAGCGGTCCGGGCAGCCCGGCAGGTTCTCCAA | PTSTEEGTSTEPSEGSA |
| | CCAGCACCGAAGAAGGTTCCCCTGCTGGTAGCCCGA | PGTSESATPESGPGTSE |
| | CCTCTACGGAGGAAGGTAGCCCTGCAGGTTCCCCAA | SATPESGPGTSESATPE |
| | CTTCTACTGAGGAAGGTACTTCTGAGTCCGCTACCC | SGPGSEPATSGSETPG |
| | CAGAAAGCGGTCCTGGTACCTCCACTGAACCGTCTG | SEPATSGSETPGSPAG |
| | AAGGCTCTGCACCAGGCACTTCTGAGTCTGCTACTC | SPTSTEEGTSTEPSEGS |
| | CAGAAAGCGGCCCAGGTTCTGAACCAGCAACTTCTG | APGTSTEPSEGSAPGS |
| | GCTCTGAGACTCCAGGCACTTCTGAGTCCGCAACGC | EPATSGSETPGSTAEA |
| | CTGAATCCGGTCCTGGTTCTGAACCAGCTACTTCCG | AGCGTAEAASASRSA |
| | GCAGCGAAACCCCAGGTACCTCTGAGTCTGCGACTC | HHHHHHHH (SEQ ID |
| | CAGAGTCTGGTCCTGGTACTTCCACTGAGCCTAGCG | NO: 714) |
| | AGGGTTCCGCACCAGGTTCTCCGGCTGGTAGCCCGA | |
| | CCAGCACGGAGGAGGGTACGTCTGAATCTGCAACG | |
| | CCGGAATCGGGCCCAGGTTCGGAGCCTGCAACGTCT | |
| | GGCAGCGAAACCCCGGGTACCTCCGAATCTGCTACA | |
| | CCGGAAAGCGGTCCTGGCAGCCCTGCTGGTTCTCCA | |
| | ACCTCTACCGAGGAGGGTTCACCGGCAGGTAGCCCG | |
| | ACTAGCACTGAAGAAGGTACTAGCACGGAGCCGAG | |
| | CGAGGGTAGTGCTCCGGGTACGAGCGAGAGCGCAA | |
| | CGCCAGAGAGCGGTCCAGGCACCAGCGAATCGGCC | |
| | ACCCCTGAGAGCGGCCCAGGTACTTCTGAGAGCGCC | |
| | ACTCCTGAATCCGGCCCTGGTAGCGAGCCGGCAACC | |
| | TCCGGCTCAGAAACTCCTGGTTCGGAACCAGCGACC | |
| | AGCGGTTCTGAAACTCCGGGTAGCCCGGCAGGCAG | |
| | CCCAACGAGCACCGAAGAGGGTACCAGCACGGAAC | |
| | CGAGCGAGGGTTCTGCCCCGGGTACTTCCACCGAAC | |
| | CATCGGAGGGCTCTGCACCTGGTAGCGAACCTGCGA | |
| | CGTCTGGTTCTGAAACGCCGGGTAgCACTGCAGAAG | |
| | CGGCTGGTtgtGGCACCGCCGAAGCAGCTagcgcctctcgct | |
| | ccgcaCATCACCATCACCACCATCATCACTAA (SEQ ID | |
| | NO: 713) | |

### Example 4

### Construction of CCD-XTEN

A pair of primers AE_003BsaIBbsIfor-TGGT&AE712-MycAgeIrev was used to run PCR on pCW1470 to obtain the PCR product of BsaIBbsI-AE712. Gel-purify the band of right size and digest with BsaI/AgeI as the insert. Ligate the insert with BsaI/AgeI digested pNL0322 vector and transform into DH5α competent cells to screen for the right clone of pCW1471 as the vector next cloning step.

A pair of primers AE42_3CBsaIfor& AE42BsaIrev-TGGT was used to run PCR on pCW1466 to obtain the PCR product of AGGT-AE42-TGGT. Gel-purify the band of right size and digest with BsaI as the insert. Digest pCW1471 above with BsaI/BbsI and gel-purify the large fragment as the vector. Ligate the vector with insert and transform into DH5α competent cells to screen by sequencing confirmation for the right clone of pCW1472 (AC1255), which encodes the protein of HD2-R-XTEN_AE42 (C8, C24, C40)-XTEN_AE712-R-Myc-H8. The DNA sequences and protein sequences are provided in the Table 29 below.

TABLE 29
DNA and amino acid sequence for 1xAmino, 3xThio-XTEN42-XTEN712

| Clone | | Amino Acid |
|---|---|---|
| Name | DNA Sequence | Sequence |
| HD2-R- | ATGAAAAACCCAGAGCAAGCAGAAGAACAAGCTGA | MKNPEQAEEQAEE |
| AE42_ | AGAACAGCGCGAAGAAACAagcgcgtctcgtGGGTCTCCAg | QREETSASRGSPGA |
| 3Cys- | gtgcAGGTAGCtgcGCTGGTAGCCCAACCTCTACCGAAG | GSCAGSPTSTEEGT |
| AE712- | AAGGTACCTCTGAATCCGCTtgTTCCCCAGAAGGTCCT | SESACSPEGPGTSTE |
| R-myc- | GGTACTAGCACTGAGCCAAGCGAAGGTTCTtgTGGCgg | PSEGSCGGPGSPAG |
| H8 | tCCtGGTAGCCCAGCTGGTAGCCCAACCTCTACCGAA | SPTSTEEGTSESATP |
| | GAAGGTACCTCTGAATCCGCTACTCCAGAATCCGGTC | ESGPGTSTEPSEGS |
| | CTGGTACTAGCACTGAGCCAAGCGAAGGTTCTGCTC | APGSPAGSPTSTEE |
| | CAGGCTCCCCGGCAGGTAGCCCTACCTCTACCGAAG | GTSTEPSEGSAPGT |
| | AGGGCACTAGCACCGAACCATCTGAGGGTTCCGCTC | STEPSEGSAPGTSES |
| | CTGGCACCTCCACTGAACCGTCCGAAGGCAGTGCTC | ATPESGPGSEPATS |
| | CGGGTACTTCCGAAAGCGCAACTCCGGAATCCGGCC | GSETPGSEPATSGS |
| | CTGGTTCTGAGCCTGCTACTTCCGGCTCTGAAACTCC | ETPGSPAGSPTSTEE |
| | AGGTAGCGAGCCAGCGACTTCTGGTTCTGAAACTCC | GTSESATPESGPGT |
| | AGGTTCACCGGCGGGTAGCCCGACGAGCACGGAGGA | STEPSEGSAPGTSTE |
| | AGGTACCTCTGAGTCGGCCACTCCTGAGTCCGGTCCG | PSEGSAPGSPAGSP |
| | GGCACGAGCACCGAGCCGAGCGAGGGTTCAGCCCCG | TSTEEGTSTEPSEGS |
| | GGTACCAGCACGGAGCCGTCCGAGGGTAGCGCACCG | APGTSTEPSEGSAP |
| | GGTTCTCCGGCGGGCTCCCCTACGTCTACGGAAGAG | GTSESATPESGPGT |
| | GGTACGTCCACTGAACCTAGCGAGGGCAGCGCGCCA | STEPSEGSAPGTSES |
| | GGCACCAGCACTGAACCGAGCGAAGGCAGCGCACCT | ATPESGPGSEPATS |
| | GGCACTAGCGAGTCTGCGACTCCGGAGAGCGGTCCG | GSETPGTSTEPSEGS |
| | GGTACGAGCACGGAACCAAGCGAAGGCAGCGCCCC | APGTSTEPSEGSAP |
| | AGGTACCTCTGAATCTGCTACCCCAGAATCTGGCCCG | GTSESATPESGPGT |
| | GGTTCCGAGCCAGCTACCTCTGGTTCTGAAACCCCAG | SESATPESGPGSPA |
| | GTACTTCCACTGAACCAAGCGAAGGTAGCGCTCCTG | GSPTSTEEGTSESAT |
| | GCACTTCTACTGAACCATCCGAAGGTTCCGCTCCTGG | PESGPGSEPATSGSE |
| | TACGTCTGAAAGCGCTACCCCTGAAAGCGGCCCAGG | TPGTSESATPESGP |
| | CACCTCTGAAAGCGCTACTCCTGAGAGCGGTCCAGG | GTSTEPSEGSAPGT |
| | CTCTCCAGCAGGTTCTCCAACCTCCACTGAAGAAGGC | STEPSEGSAPGTSTE |
| | ACCTCTGAGTCTGCTACCCCTGAATCTGGTCCTGGCT | PSEGSAPGTSTEPSE |
| | CCGAACCTGCTACCTCTGGTTCCGAAACTCCAGGTAC | GSAPGTSTEPSEGS |
| | CTCGGAATCTGCGACTCCGGAATCTGGCCCGGGCAC | APGTSTEPSEGSAP |
| | GAGCACGGAGCCGTCTGAGGGTAGCGCACCAGGTAC | GSPAGSPTSTEEGT |
| | CAGCACTGAGCCTTCTGAGGGCTCTGCACCGGGTAC | STEPSEGSAPGTSES |
| | CTCCACGGAACCTTCGGAAGGTTCTGCGCCGGGTAC | ATPESGPGSEPATS |
| | CTCCACTGAGCCATCCGAGGGTTCAGCACCAGGTAC | GSETPGTSESATPES |
| | TAGCACGGAACCGTCCGAGGGCTCTGCACCAGGTAC | GPGSEPATSGSETP |
| | GAGCACCGAACCGTCGGAGGGTAGCGCTCCAGGTAG | GTSESATPESGPGT |
| | CCCAGCGGGCTCTCCGACAAGCACCGAAGAAGGCAC | STEPSEGSAPGTSES |
| | CAGCACCGAGCCGTCCGAAGGTTCCGCACCAGGTAC | ATPESGPGSPAGSP |
| | AAGCGAGAGCGCGACTCCTGAATCTGGTCCGGGTAG | TSTEEGSPAGSPTST |
| | CGAGCCTGCAACCAGCGGTTCTGAGACGCCGGGCAC | EEGSPAGSPTSTEE |
| | TTCCGAATCTGCGACCCCGGAGTCCGGTCCAGGTTCA | GTSESATPESGPGT |
| | GAGCCGGCGACGAGCGGTTCGGAAACGCCGGGTACG | STEPSEGSAPGTSES |
| | TCTGAATCAGCCACGCCGGAGTCTGGTCCGGGTACCT | ATPESGPGSEPATS |
| | CGACCGAACCAAGCGAAGGTTCGGCACCGGGTACTA | GSETPGTSESATPES |
| | GCGAGAGCGCAACCCCTGAAAGCGGTCCGGGCAGCC | GPGSEPATSGSETP |
| | CGGCAGGTTCTCCAACCAGCACCGAAGAAGGTTCCC | GTSESATPESGPGT |
| | CTGCTGGTAGCCCGACCTCTACGGAGGAAGGTAGCC | STEPSEGSAPGSPA |
| | CTGCAGGTTCCCCAACTTCTACTGAGGAAGGTACTTC | GSPTSTEEGTSESAT |
| | TGAGTCCGCTACCCCAGAAAGCGGTCCTGGTACCTCC | PESGPGSEPATSGSE |
| | ACTGAACCGTCTGAAGGCTCTGCACCAGGCACTTCTG | TPGTSESATPESGP |
| | AGTCTGCTACTCCAGAAAGCGGCCCAGGTTCTGAAC | GSPAGSPTSTEEGSP |
| | CAGCAACTTCTGGCTCTGAGACTCCAGGCACTTCTGA | ASASRSAEQKLISE |
| | GTCCGCAACGCCTGAATCCGGTCCTGGTTCTGAACCA | EDLSPATGHHHHH |
| | GCTACTTCCGGCAGCGAAACCCCAGGTACCTCTGAG | HHH (SEQ ID NO: |
| | TCTGCGACTCCAGAGTCTGGTCCTGGTACTTCCACTG | 716) |
| | AGCCTAGCGAGGGTTCCGCACCAGGTTCTCCGGCTG | |
| | GTAGCCCGACCAGCACGGAGGAGGGTACGTCTGAAT | |
| | CTGCAACGCCGGAATCGGGCCCAGGTTCGGAGCCTG | |
| | CAACGTCTGGCAGCGAAACCCCGGGTACCTCCGAAT | |
| | CTGCTACACCGGAAAGCGGTCCTGGCAGCCCTGCTG | |
| | GTTCTCCAACCTCTACCGAGGAGGGTTCACCGGCAag | |
| | cgcctctcgctccgcaGAACAGAAACTGATCTCTGAAGAGGA | |
| | TCTGtctccggctaccggtCATCACCATCACCACCATCATCAC | |
| | TAA (SEQ ID NO: 715) | |

### Example 5

### Construction of CCD-PCM-XTEN

A pair of primers 4Afor&AE712HindIIIrev was used to run the PCR reaction on plasmid pCW1464 containing PCM-XTEN_AE712-H8 to obtain the PCR product of AE712 without Histidines. Gel-purify the band of right size and digest with SbfI/HindIII as the insert. Digest pCW1464 with BsaI/HindIII and gel-purify the large fragment as the vector. Ligate the vector with the previously BsaI/SbfI digested XTEN fragment from pCW1330 and SbfI/HindIII digested PCR insert of XTEN_AE712. Transform into DH5α competent cells to obtain the colonies of pCW1469 and screen for the right clone by sequencing confirmation as the vector in next cloning step.

A pair of primers TEV-AE42_3CNheIfor&AE42_3CBsaIrev was used to run PCR on pCW1466 to obtain the PCR product of TEV-AE42_3C. Gel-purify the band of right size and digest with NheI/BsaI as the insert. Digest plasmid pCW1469 above with NdeI/BsaI and gel-purify the large fragment as the vector. Ligate the vector with the annealed oligonucleotides of HD2-H8NheIfor&rev and the NheI/BsaI digested PCR insert of TEV-AE42_3C. Transform into DH5α competent cells to screen by sequencing confirmation for the right clone of pCW1470 (AC1254), which encodes the protein of HD2-His8-TEV-XTEN_AE42 (C8, C24, C40)-PCM-XTEN_AE712 (“His8” disclosed as SEQ ID NO: 721). The DNA sequences and protein sequences are provided in the Table 30, below.

TABLE 30
DNA and amino acid sequence for 1xAmino, 3xThio-XTEN42-PCM-XTEN712

| Clone | | Amino Acid |
|---|---|---|
| Name | DNA Sequence | Sequence |
| HD2-H8- | ATGAAAAACCCAGAGCAAGCAGAAGAACAAGCTGA | MKNPEQAEEQAEE |
| TEV- | AGAACAGCGCGAAGAAACACACCATCACCATCATCA | QREETHHHHHHHH |
| AE42_ | CCACCATtctgctagccgaccgctGAAAATCTGTATTTTCAGG | SASRSAENLYFQGS |
| 3Cys-PCM- | GcTCTCCAggtgcAGGTAGCtgcGCTGGTAGCCCAACCTC | PGAGSCAGSPTSTE |
| AE712 | TACCGAAGAAGGTACCTCTGAATCCGCTtgTTCCCCA | EGTSESACSPEGPG |
| | GAAGGTCCTGGTACTAGCACTGAGCCAAGCGAAGGT | TSTEPSEGSCGGLS |
| | TCTtgTGGCggtctgtCAggtCGTtctGATaacCATtccCCActgGG | GRSDNHSPLGLAGS |
| | TctgGCTGGGTCTCCAGGTAGCCCAGCTGGTAGCCCAA | PGSPAGSPTSTEEG |
| | CCTCTACCGAAGAAGGTACCTCTGAATCCGCTACTCC | TSESATPESGPGTST |
| | AGAATCCGGTCCTGGTACTAGCACTGAGCCAAGCGA | EPSEGSAPGSPAGS |
| | AGGTTCTGCTCCAGGCTCCCCGGCAGGTAGCCCTACC | PTSTEEGTSTEPSEG |
| | TCTACCGAAGAGGGCACTAGCACCGAACCATCTGAG | SAPGTSTEPSEGSAP |
| | GGTTCCGCTCCTGGCACCTCCACTGAACCGTCCGAAG | GTSESATPESGPGS |
| | GCAGTGCTCCGGGTACTTCCGAAAGCGCAACTCCGG | EPATSGSETPGSEP |
| | AATCCGGCCCTGGTTCTGAGCCTGCTACTTCCGGCTC | ATSGSETPGSPAGS |
| | TGAAACTCCAGGTAGCGAGCCAGCGACTTCTGGTTCT | PTSTEEGTSESATPE |
| | GAAACTCCAGGTTCACCGGCGGGTAGCCCGACGAGC | SGPGTSTEPSEGSAP |
| | ACGGAGGAAGGTACCTCTGAGTCGGCCACTCCTGAG | GTSTEPSEGSAPGSP |
| | TCCGGTCCGGGCACGAGCACCGAGCCGAGCGAGGGT | AGSPTSTEEGTSTEP |
| | TCAGCCCCGGGTACCAGCACGGAGCCGTCCGAGGGT | SEGSAPGTSTEPSE |
| | AGCGCACCGGGTTCTCCGGCGGGCTCCCCTACGTCTA | GSAPGTSESATPES |
| | CGGAAGAGGGTACGTCCACTGAACCTAGCGAGGGCA | GPGTSTEPSEGSAP |
| | GCGCGCCAGGCACCAGCACTGAACCGAGCGAAGGCA | GTSESATPESGPGS |
| | GCGCACCTGGCACTAGCGAGTCTGCGACTCCGGAGA | EPATSGSETPGTSTE |
| | GCGGTCCGGGTACGAGCACGGAACCAAGCGAAGGC | PSEGSAPGTSTEPSE |
| | AGCGCCCCAGGTACCTCTGAATCTGCTACCCCAGAAT | GSAPGTSESATPES |
| | CTGGCCCGGGTTCCGAGCCAGCTACCTCTGGTTCTGA | GPGTSESATPESGP |
| | AACCCCAGGTACTTCCACTGAACCAAGCGAAGGTAG | GSPAGSPTSTEEGT |
| | CGCTCCTGGCACTTCTACTGAACCATCCGAAGGTTCC | SESATPESGPGSEPA |
| | GCTCCTGGTACGTCTGAAAGCGCTACCCCTGAAAGC | TSGSETPGTSESATP |
| | GGCCCAGGCACCTCTGAAAGCGCTACTCCTGAGAGC | ESGPGTSTEPSEGS |
| | GGTCCAGGCTCTCCAGCAGGTTCTCCAACCTCCACTG | APGTSTEPSEGSAP |
| | AAGAAGGCACCTCTGAGTCTGCTACCCCTGAATCTG | GTSTEPSEGSAPGT |
| | GTCCTGGCTCCGAACCTGCTACCTCTGGTTCCGAAAC | STEPSEGSAPGTSTE |
| | TCCAGGTACCTCGGAATCTGCGACTCCGGAATCTGGC | PSEGSAPGTSTEPSE |
| | CCGGGCACGAGCACGGAGCCGTCTGAGGGTAGCGCA | GSAPGSPAGSPTST |
| | CCAGGTACCAGCACTGAGCCTTCTGAGGGCTCTGCA | EEGTSTEPSEGSAP |
| | CCGGGTACCTCCACGGAACCTTCGGAAGGTTCTGCG | GTSESATPESGPGS |
| | CCGGGTACCTCCACTGAGCCATCCGAGGGTTCAGCA | EPATSGSETPGTSES |
| | CCAGGTACTAGCACGGAACCGTCCGAGGGCTCTGCA | ATPESGPGSEPATS |
| | CCAGGTACGAGCACCGAACCGTCGGAGGGTAGCGCT | GSETPGTSESATPES |
| | CCAGGTAGCCCAGCGGGCTCTCCGACAAGCACCGAA | GPGTSTEPSEGSAP |
| | GAAGGCACCAGCACCGAGCCGTCCGAAGGTTCCGCA | GTSESATPESGPGSP |
| | CCAGGTACAAGCGAGAGCGCGACTCCTGAATCTGGT | AGSPTSTEEGSPAG |
| | CCGGGTAGCGAGCCTGCAACCAGCGGTTCTGAGACG | SPTSTEEGSPAGSPT |
| | CCGGGCACTTCCGAATCTGCGACCCCGGAGTCCGGT | STEEGTSESATPESG |
| | CCAGGTTCAGAGCCGGCGACGAGCGGTTCGGAAACG | PGTSTEPSEGSAPG |
| | CCGGGTACGTCTGAATCAGCCACGCCGGAGTCTGGT | TSESATPESGPGSEP |
| | CCGGGTACCTCGACCGAACCAAGCGAAGGTTCGGCA | ATSGSETPGTSESA |
| | CCGGGTACTAGCGAGAGCGCAACCCCTGAAAGCGGT | TPESGPGSEPATSGS |
| | CCGGGCAGCCCGGCAGGTTCTCCAACCAGCACCGAA | ETPGTSESATPESGP |
| | GAAGGTTCCCCTGCTGGTAGCCCGACCTCTACGGAG | GTSTEPSEGSAPGSP |
| | GAAGGTAGCCCTGCAGGTTCCCCAACTTCTACTGAG | AGSPTSTEEGTSES |
| | GAAGGTACTTCTGAGTCCGCTACCCCAGAAAGCGGT | ATPESGPGSEPATS |
| | CCTGGTACCTCCACTGAACCGTCTGAAGGCTCTGCAC | GSETPGTSESATPES |
| | CAGGCACTTCTGAGTCTGCTACTCCAGAAAGCGGCC | GPGSPAGSPTSTEE |
| | CAGGTTCTGAACCAGCAACTTCTGGCTCTGAGACTCC | GSPA (SEQ ID NO: |
| | AGGCACTTCTGAGTCCGCAACGCCTGAATCCGGTCCT | 718) |
| | GGTTCTGAACCAGCTACTTCCGGCAGCGAAACCCCA | |
| | GGTACCTCTGAGTCTGCGACTCCAGAGTCTGGTCCTG | |
| | GTACTTCCACTGAGCCTAGCGAGGGTTCCGCACCAG | |
| | GTTCTCCGGCTGGTAGCCCGACCAGCACGGAGGAGG | |
| | GTACGTCTGAATCTGCAACGCCGGAATCGGGCCCAG | |
| | GTTCGGAGCCTGCAACGTCTGGCAGCGAAACCCCGG | |
| | GTACCTCCGAATCTGCTACACCGGAAAGCGGTCCTG | |
| | GCAGCCCTGCTGGTTCTCCAACCTCTACCGAGGAGG | |
| | GTTCACCGGCATAA (SEQ ID NO: 717) | |

### Example 6

### Fermentation of XTEN for Conjugation

Starter cultures were prepared by inoculating glycerol stocks of E. coli carrying the plasmid containing the appropriate XTEN for conjugation protein sequences into 125 mL of LB Broth media containing 50 μg/mL kanamycin. The cultures were then shaken overnight at 37° C. The starter culture was used to inoculate 2 L of fermentation batch media containing −12.5 g ammonium sulfate, 15 g potassium phosphate dibasic anhydrous, 2.5 g sodium citrate dihydrate; 8.5 g sodium phosphate monobasic monohydrate; 50 g NZ BL4 soy peptone (Kerry Bioscience #5X00043); 25 g yeast extract (Teknova #Y9020); 1.8 L water; 0.5 mL polypropylene glycol; 2.5 mL trace elements solution (Amunix recipe 144-1); 17.5 mL 1M magnesium sulfate; and 2 mL Kanamycin (50 mg/mL)—in 5 L glass jacketed vessel with a B. Braun Biostat B controller. The fermentation control settings were: pH=6.9+/−0.1; dO2=10%; dissolved oxygen cascade in stirrer only mode with a range of 125-1180 rpm; air flow of 5 liters per minute of 90% oxygen; initial temperature 37° C.; base control 13% ammonium hydroxide; and no acid control. After 6 hours of culture a 50% glucose feed was initiated at a rate of 30 g/hr. After 20 hours of culture, 25 mL of 1M magnesium sulfate and 3 mL of 1M IPTG were added. After a total fermentation run time of 45 hours the culture was harvested by centrifugation yielding cell pellets between 0.45-1.1 kilograms in wet weight for all constructs. The pellets were stored frozen at −80° C. until further use. Culture samples at multiple time points in the fermentation were taken, the cells were lysed, then cell debris was flocculated with heat and rapid cooling, clarified soluble lysates were prepared by centrifugation and analyzed by a regular non-reducing SDS-PAGE using NuPAGE 4-12% Bis-Tris gel from Invitrogen according to manufacturer's specifications with Coomassie staining Results show an accumulation of XTEN fusion protein as a function of fermentation run time and that the XTEN fusion protein constructs were expressed at fermentation scale with titers >1 g/L, with an apparent MW of about 160 kDa (note: the actual molecular weight are 100 kDa. The observed migration in SDS-PAGE was comparable to that observed for other XTEN-containing fusion proteins).

### Example 7

### Purification of CCD-XTEN from High-Density Bacterial Fermentation

1. Expression

The construct AC1255 (MKNPEQAEEQAEEQREET-SASRGS-CCD1-XTEN_AE713-SASRSA-Myc-His8) (“MKNPEQAEEQAEEQREET,” “SASRGS,” “SASRSA,” and “His8” disclosed as SEQ ID NOS 719-720, 218, and 721, respectively) was expressed in E. coli BL21_DE3 under the control of T7 RNA polymerase. AC1255 was cultured in a LB flask until OD600 of ˜2.5 and transferred into a 10 L fermenter containing a rich medium with 2.1 g/L glucose. After batch feed exhaustion, 70% w/v glucose was added in a pre-programmed glucose limited profile. The culture was induced with IPTG at 40-50 OD600 then cultured for 18-24 hours until harvest. The cells were pelleted by centrifugation and frozen at −80° C.

2. Lysis and Clarification

The cell pellet (2600 g) was resuspended in 7400 ml of 50 mM citrate, pH 4.0. The resuspended cells were heated to 80° C. for 15 minutes, followed by rapid cooling on ice for 30 minutes. pH of the lysate was adjusted to 3.0 with 12 M HCl, and the lysate was stored overnight at pH 3.0 with stirring at 4° C. The lysate was then clarified by centrifugation at 7000 rpm for 40 min at 4° C. for two times, and the supernatant was 0.2 μm filtered.

3. Cation Exchange Capture Step

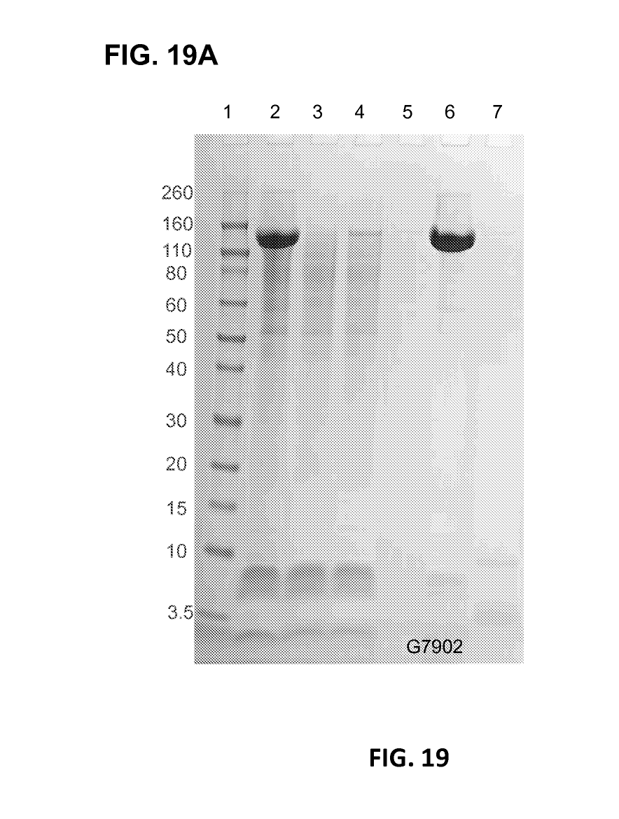

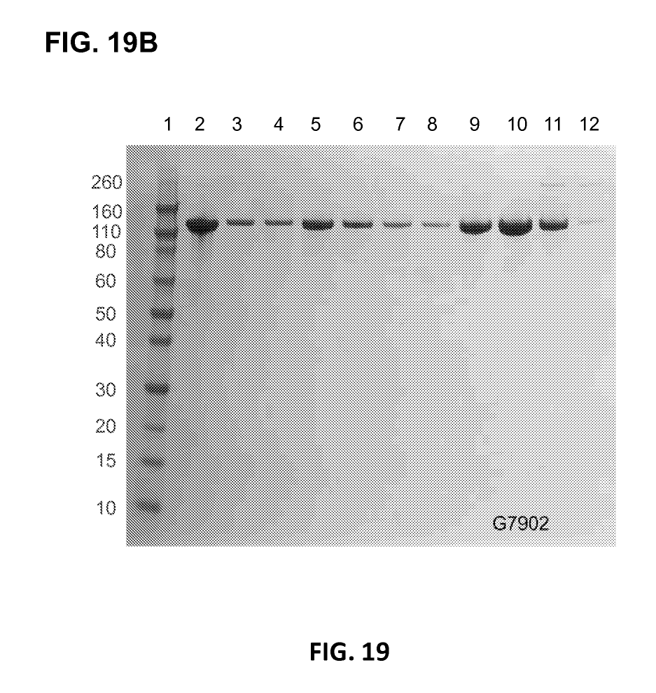

The clarified lysate was loaded on to a 1 L column packed with Capto SP ImpRes (GE Healthcare), previously sanitized with NaOH and equilibrated with Mcilvaine's Buffer, pH 3.0, 100 mM NaCl. The column was washed with 3 column volumes of Mcilvaine's Buffer, pH 3.0, and eluted with 5 column volume of elution buffer (34% 20 mM citric acid, pH 2.5, 66% 40 mM sodium phosphate dibasic, pH 9.0), and then stripped with 1 column volume of 20 M phosphate, 500 mM NaCl, pH 7.0. The fractions were analyzed by 4-12% Bis-Tris SDS-PAGE and Coomassie staining (FIG. 19A). Based on the gel, the elution fraction contained desired product and was used for further processing.

4. Trypsin Digestion of Cation Exchange Column Elution Pool

After cation exchange chromatography, trypsin digestion was used to cleave off the N-tag and C-tag. pH of the pooled fractions was then adjusted to 8.0 using 40% w/w sodium hydroxide/12 M HCl, and then incubated with bovine trypsin (Sigma) at room temperature overnight with gentle mixing (1:5000 enzyme/protein mass ratio). After reaction, trypsin was inactivated by adding 2 mM EDTA and 10 mM DTT, and heating the reaction mixture to 80° C. for 15 minutes, followed by cooling on ice till the temperature was decreased to 10° C.

5. Anion Exchange Polishing Step

Anion exchange chromatography was used as a polishing step to separate the cleaved tags and truncated products from the final product. The sample after trypsin digestion was 0.2 μm filtered, and then loaded on to a 1.57 L column packed with Capto Q ImpRes (GE Healthcare), previously sanitized with NaOH and equilibrated with 20 mM MES, pH 6.35. After loading, the column was washed with 4 column volumes of 20 mM MES pH 6.35, and then 4 column volumes of 20 mM MES, pH 6.35, 50 mM NaCl. Three gradient elution steps were applied: 20 mM MES, pH 6.35, 50-145 mM NaCl over 5 column volumes, followed by 20 mM MES, pH 6.35, 145-205 mM NaCl over 5 column volumes, and finally 20 mM MES, pH 6.35, 205-350 mM NaCl over 5 column volumes. After that, the column was stripped with 20 mM MES, pH 6.35, 500 mM NaCl for 0.5 column volume. The fractions were analyzed by SDS-PAGE followed by staining by Stains-all (FIG. 19B). Based on the gel, elutions E1 to E7 were pooled for formulation.

7. Concentration and Buffer Exchange (Formulation)

As a final step, the pooled proteins were buffer exchanged into the formulation buffer (20 mM MES, pH 5.5) using Biomax-5 Pellicon XL ultrafiltration cassettes (Millipore) and concentrated down to a final concentration of >15 mg/mL.

### Example 8

### Purification of CCD-PCM-XTEN from High-Density Bacterial Fermentation

1. Expression

The construct AC1254 (MKNPEQAEEQAEEQREET-His8-SASRSA-TEV-CCD1-LSGRSDNHSPLGLAGS-AE713) (“MKNPEQAEEQAEEQREET,” “His8,” “SASRSA,” and “LSGRSDNHSPLGLAGS” disclosed as SEQ ID NOS 719, 721, 218, and 97, respectively) was expressed in E. coli BL21_DE3 under the control of T7 RNA polymerase. AC1255 was cultured in a LB flask until OD600 of ˜2.5 and transferred into a 10 L fermenter containing a rich medium with 2.1 g/L glucose. After batch feed exhaustion, 70% w/v glucose was added in a pre-programmed glucose limited profile. The culture was induced with IPTG at 40-50 OD600 then cultured for 18-24 hours until harvest. The cells were pelleted by centrifugation and frozen at −80° C.

2. Lysis and Clarification

The cell pellet (4204 g) was resuspended in 8400 ml of 50 mM citrate, pH 4.0. The resuspended cells were heated to 80° C. for 15 minutes, followed by rapid cooling on ice for 30 minutes. pH of the lysate was adjusted to 3.0 with 12 M HCl, and the lysate was stored overnight at pH 3.0 with stirring at 4° C. The lysate was then clarified by centrifugation at 7000 rpm for 40 min at 4° C. for two times, and the supernatant was 0.2 μm filtered.

3. Cation Exchange Capture Step

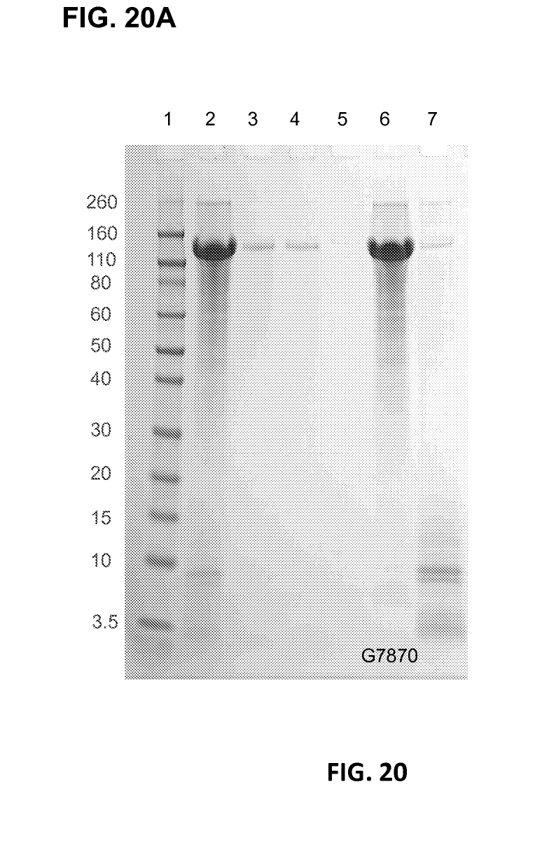

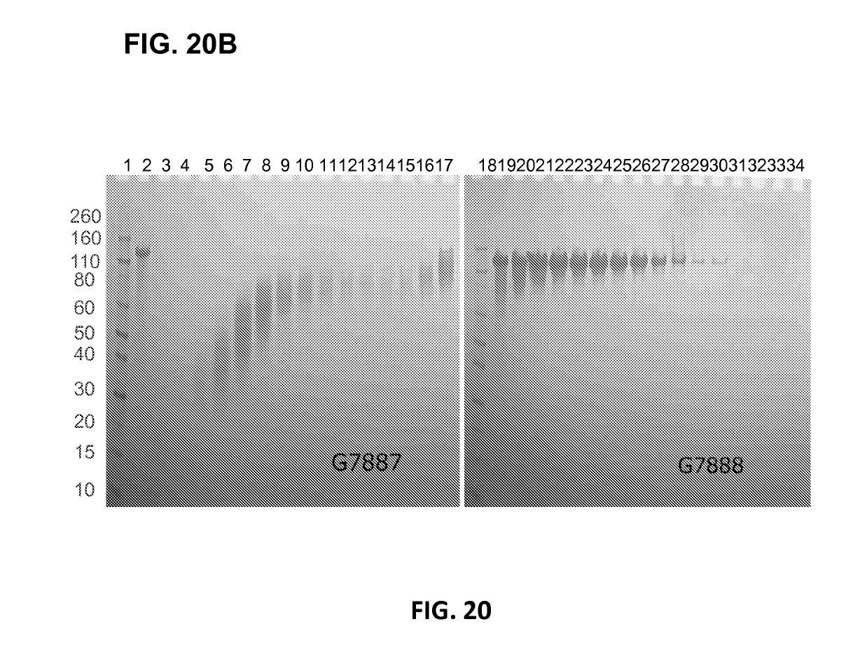

The clarified lysate was loaded on to a 1 L column packed with Capto SP ImpRes (GE Healthcare), previously sanitized with NaOH and equilibrated with Mcilvaine's Buffer, pH 3.0, 100 mM NaCl. The column was washed with 3 column volumes of Mcilvaine's Buffer, pH 3.0, and eluted with 5 column volume of elution buffer (34% 20 mM citric acid, pH 2.5, 66% 40 mM sodium phosphate dibasic, pH 9.0), and then stripped with 1 column volume of 20 M phosphate, 500 mM NaCl, pH 7.0. The fractions were analyzed by 4-12% Bis-Tris SDS-PAGE and Coomassie staining (FIG. 20A). Based on the gel, the elution fraction contained desired product and was used for further processing.

4. TEV Protease Digestion of Cation Exchange Column Elution Pool

After cation exchange chromatography, TEV protease digestion was used to cleave off the N-tag. pH of the elution pool was then adjusted to pH 8.0 using 40% w/w sodium hydroxide, and DTT and EDTA were added to the sample to reach final concentration of 1 mM each. Then, the sample was incubated with TEV protease at room temperature overnight with gentle mixing (1:20 enzyme/protein mass ratio). After reaction, TEV protease was inactivated by adding 2 mM EDTA and 10 mM DTT, and heating the reaction mixture to 80° C. for 15 minutes, followed by cooling on ice till the temperature was decreased to 10° C.

5. Anion Exchange Polishing Step

Anion exchange chromatography was used as a polishing step to separate the cleaved tags and truncated products from the final product. The sample after TEV digestion was 0.2 μm filtered, and then loaded on to a 3 L column packed with Capto Q ImpRes (GE Healthcare), previously sanitized with NaOH and equilibrated with 20 mM MES, pH 6.35. After loading, the column was washed with 3 column volumes of 20 mM MES pH 6.35, and 3 column volumes of 20 mM MES, pH 6.35, 40 mM NaCl. Three gradient elution steps were then applied: 20 mM MES, pH 6.35, 40-90 mM NaCl over 3 column volumes, followed by 20 mM MES, pH 6.35, 90-200 mM NaCl over 5 column volumes, and finally 20 mM MES, pH 6.35, 200-350 mM NaCl over 8 column volumes. The column was then stripped with 20 mM MES, pH 6.35, 500 mM NaCl for 1 column volume. The fractions were analyzed by SDS-PAGE followed by staining by Stains-all (FIG. 20B). Based on the gel, elutions 17 to 22 were pooled for formulation.

7. Concentration and Buffer Exchange (Formulation)

As a final step, the pooled proteins were buffer exchanged into the formulation buffer (20 mM MES, pH 5.5) using Biomax-5 Pellicon XL ultrafiltration cassettes (Millipore) and concentrated down to a final concentration of >15 mg/mL.

### Example 9

### Enzyme Activation, Storage and Digestion of CCD-PCM-XTEN Construct AC1254

The example shows that one of the aforementioned CCD-PCM-XTEN constructs AC1254, with PCM sequence being BSRS1 in Table 8 and previously described in Example 8, can be cleaved by various tumor-associated proteases including MMP-2, MMP-7, MMP-9, MMP-14, MTSP1, and uPA in test tubes.

1. Enzyme Activation

All enzymes used were obtained from R&D Systems. Recombinant human u-plasminogen activator (uPA) and recombinant human matriptase were provided as activated enzymes and stored at −80° C. until use. Recombinant mouse MMP-2, recombinant human MMP-7, and recombinant mouse MMP-9 were supplied as zymogens and required activation by 4-aminophenylmercuric acetate (APMA). APMA was first dissolved in 0.1M NaOH to a final concentration of 10 mM before the pH was readjusted to neutral using 0.1N HCl. Further dilution of the APMA stock to 2.5 mM was done in 50 mM Tris, 150 mM NaCl, 10 mM CaCl₂, pH 7.5. To activate pro-MMP, 1 mM APMA and 100 ug/mL of pro-MMP were incubated at 37° C. for 1 hour (MMP-2, MMP-7) or 3 hours (MMP-9). Activated enzyme added to a fmal concentration of 50% glycerol could then be stored at −20° C. for several weeks.

2. Enzymatic Digestion

A panel of enzymes was tested to determine cleavage efficiency of each enzyme for AC1254 (CCD1-BSRS1-AE713). 10 μM of the substrate was incubated with each enzyme in the following enzyme-to-substrate molar ratios: MMP-2 (1:680), MMP-7 (1:200), MMP-9 (1: 6711), matriptase (1:12.5), and uPA (1:12.5). Reactions were incubated at 37° C. for two hours before stopping digestion by adding EDTA to 20 mM in the case of MMP reactions and heating at 85° C. for 15 minutes in the case of uPA and matriptase reactions.

3. Analysis of Cleavage Efficiency.

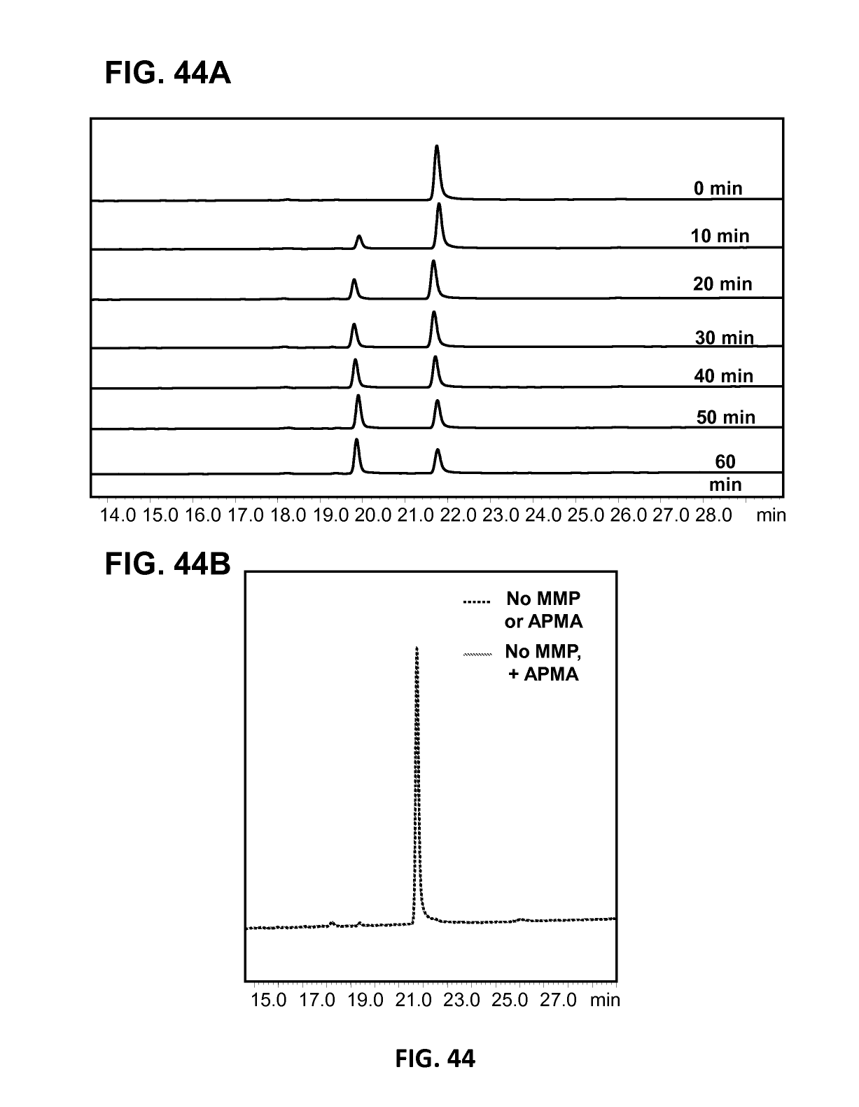

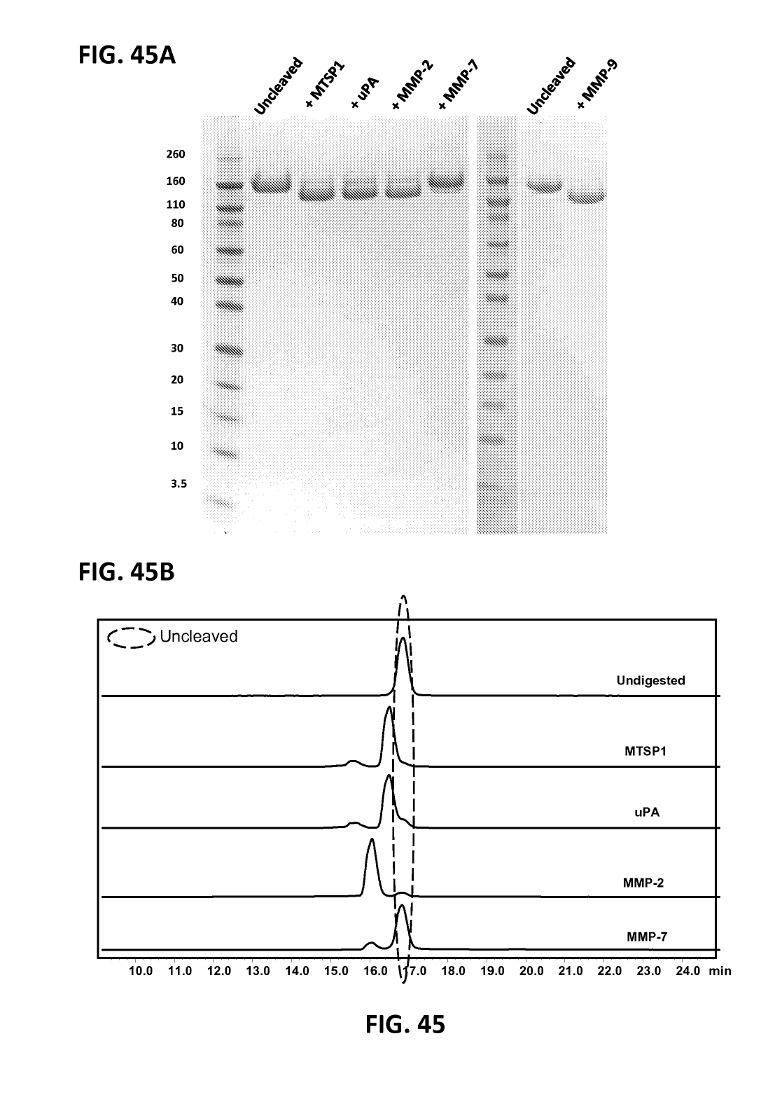

Analysis of the samples to determine percentage of cleaved product was performed by loading 5 μg of undigested and digested material on SDS-PAGE and staining with Coomassie Blue (FIG. 45A), as well as running 8 μg of undigested and digested product to C4 RP-HPLC (FIG. 45B). Through RP-HPLC, a typical time course of MMP-9 digestion over the course of an hour could be observed through analyzing samples of the digestion reaction collected at 10 minute intervals. (FIG. 44A) Two negative controls could also be included: one to confirm that digestion did not occur in the absence of MMP, and one to confirm that digestion did not occur in the presence of APMA alone (FIG. 44B). For SDS-PAGE gels, ImageJ was used to analyze band intensity and determine percent cleavage. Upon cleavage by various proteases at the PCM segment, the substrate CCD1-PCM-XTEN would yield two fragments, with the small fragment mostly containing CCD1 and the other containing XTEN, which would migrate at a lower apparent molecular weight on SDS-PAGE. The results confirm that all proteases tested here cleaved the construct as intended, with varying degree of catalytic efficiency.

### Example 10

### Conjugation of IA-MMAE to CXTEN864 to Produce 3x-MMAE-CXTEN864

205 mg of CXTEN (XTEN_AE864(Am1,C12,C432,C854), sequence in Table 31, below) was reduced with 3 molar equivalents of TCEP at pH 8.0 for 20 min at 80° C. The reduced 3x-Cys-XTEN was reacted with 5 molar equivalents of IA-MMAE (dissolved in anhydrous DMF) overnight at 25° C. Conjugation efficiency was assessed by C4 RP-HPLC. Quantification of the separation between the fully conjugated drug-loaded product peak (in this case the 3x-MMAE peak) and the underconjugated peak closest to the fully conjugated peak (in this case the 2x-MMAE peak) was determined by computing the Peak Separation as the difference in retention time between these peaks divided by the full width at half maximum, using the following formula:

Peak Separation=(t_{R2}−t_{R1})/FWHM

wherein

t_{R2}: retention time of fully conjugated product peak

t_{R1}: retention time of underconjugated peak that is the closest to the fully conjugated product peak

FWHM: full width at half maximum

Analytical method for comparison of separation for different constructs: reaction mixture containing 10 μg XTEN was injected to C4 RP-HPLC (Vydac, catalog #214TP5415, 4.6 mm×150 mm, 5 um particle size)) and analyzed using a method of 5-50% Buffer B (0.1% TFA in acetonitrile) in Buffer A (0.1% TFA in water) over 45 minutes at 1 mL/min.

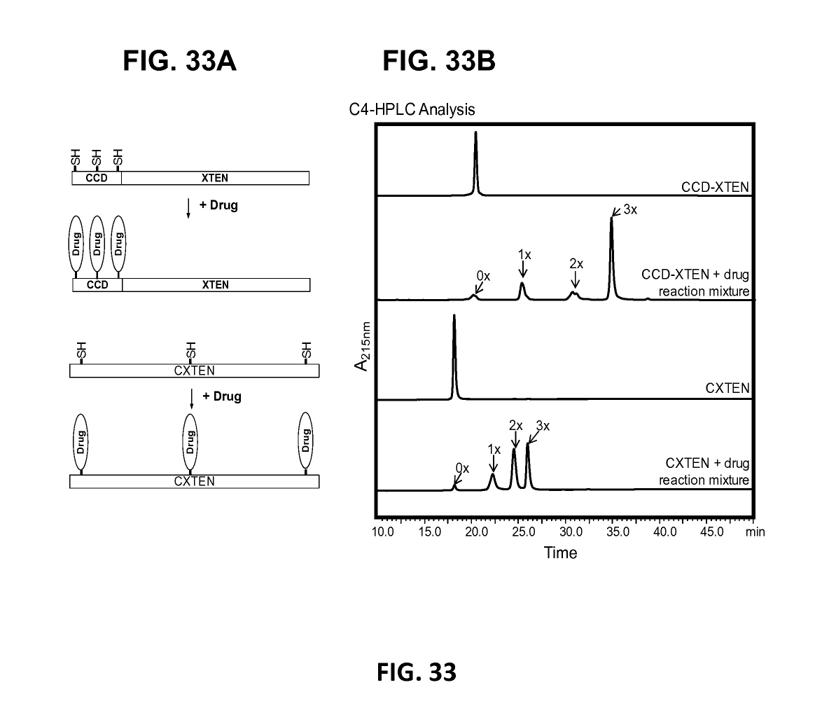

Using this method, Peak Separation was determined to be 4.5 for CXTEN when making 3xMMAE conjugate (FIG. 33B).

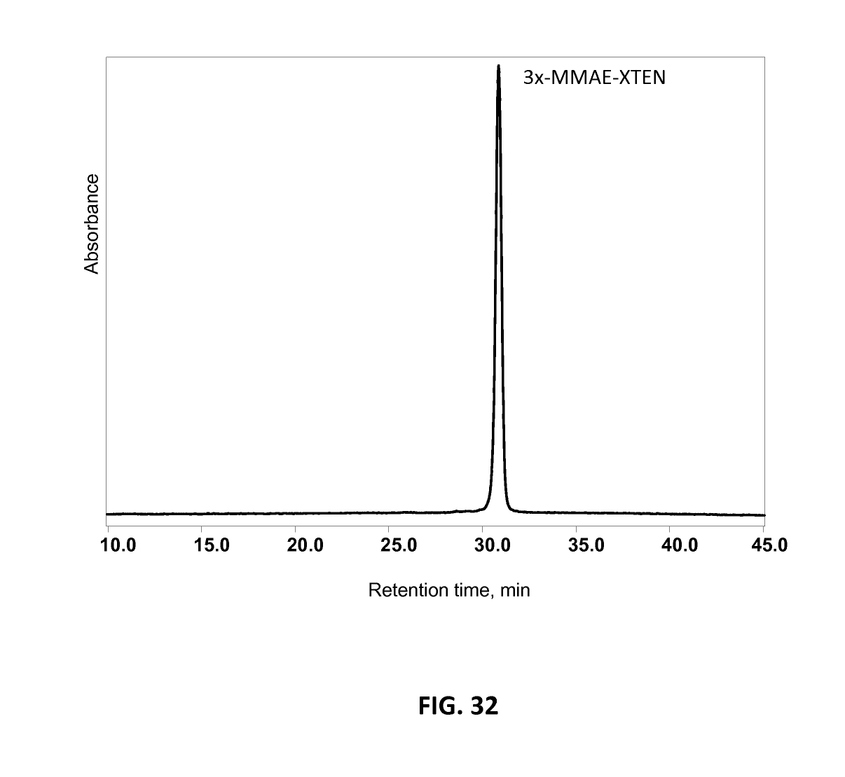

For purification, the mixture was acidified to pH <3 with TFA, and DMF was added to final volume of 13% (v/v). The mixture was split into two aliquots, and each was purified by preparative C4 RP-HPLC (C4, Vydac, 250 mm×22 mm). Chromatographic fractions were analyzed by C4 RP-HPLC. Fractions containing the desired product were pooled, neutralized with 1 M HEPES pH 8.0, and concentrated under vacuum. The 3x-MMAE-XTEN product was formulated into 20 mM HEPES pH 7.0, 50 mM NaCl using ultrafiltration (Sartorius, Vivacell 100, 10 kDa MWCO). High purity of the final product was demonstrated by C4 RP-HPLC (>95%, FIG. 32). The reaction yield was determined to be 53.0%.

TABLE 31
Amino acid sequence of components in Example 10

| Component Name | Amino Acid Sequence |
|---|---|
| XTEN_AE864 (Am1, C12, | SAGSPTAEAAGCGTAEAATSESATPESGPGTSTEPSEGSAP |
| C432, C854) | GSPAGSPTSTEEGTSTEPSEGSAPGTSTEPSEGSAPGTSESA |
| | TPESGPGSEPATSGSETPGSEPATSGSETPGSPAGSPTSTEE |
| | GTSESATPESGPGTSTEPSEGSAPGTSTEPSEGSAPGSPAGS |
| | PTSTEEGTSTEPSEGSAPGTSTEPSEGSAPGTSESATPESGP |
| | GTSTEPSEGSAPGTSESATPESGPGSEPATSGSETPGTSTEPS |
| | EGSAPGTSTEPSEGSAPGTSESATPESGPGTSESATPESGPG |
| | SPAGSPTSTEEGTSESATPESGPGSEPATSGSETPGTSESATP |
| | ESGPGTSTEPSEGSAPGTSTEPSEGSAPGTSTEPSEGSAPGT |
| | STEPSEGSAPGTSTEPSEGSAPGTSTEPSEGSAPGSPAGSPTS |
| | TEETAEAAGCGTAEAATSESATPESGPGSEPATSGSETPGT |
| | SESATPESGPGSEPATSGSETPGTSESATPESGPGTSTEPSEG |
| | SAPGTSESATPESGPGSPAGSPTSTEEGSPAGSPTSTEEGSP |
| | AGSPTSTEEGTSESATPESGPGTSTEPSEGSAPGTSESATPE |
| | SGPGSEPATSGSETPGTSESATPESGPGSEPATSGSETPGTS |
| | ESATPESGPGTSTEPSEGSAPGSPAGSPTSTEEGTSESATPES |
| | GPGSEPATSGSETPGTSESATPESGPGSPAGSPTSTEEGSPA |
| | GSPTSTEEGTSTEPSEGSAPGTSESATPESGPGTSESATPES |
| | GPGTSESATPESGPGSEPATSGSETPGSEPATSGSETPGSPA |
| | GSPTSTEEGTSTEPSEGSAPGTSTEPSEGSAPGSEPATSGSE |
| | TPGSTAEAAGCGTAEAASASR (SEQ ID NO: 722) |

### Example 11

### Conjugation of IA-MMAE to CCD-XTEN to Produce 3x-MMAE-CCD-XTEN

To 236 mg of CCD-XTEN (XTEN_AE759(Am1,C8,C24,C40), sequence in Table 33, below) in 12.3 mL of 20 mM MES pH 5.5 was added 3.08 mL 1 M HEPES pH 8.0. This CCD-XTEN was reduced with 3 molar equivalents of TCEP for 20 min at 80° C. The reduced CCD-XTEN was then diluted with 1.4 mL of 1 M HEPES pH 8.0 and reacted with 6 molar equivalents of IA-MMAE (dissolved in 6.13 mL anhydrous DMF) for 24 h at 25° C. The reaction was quenched with 30 molar equivalents of glutathione for 30 min at 25° C.

Conjugation was assessed by C4 RP-HPLC (Vydac, catalog #214TP5415, 4.6 mm×150 mm, 5 um particle size) using the same analytical method as described in Example 10. Improved Peak Separation (Peak Separation=11.8) for 3x drug-loaded CCD-XTEN was observed (FIG. 33B), compared to 3x drug-loaded CXTEN (Peak Separation=4.5, see Example 10).

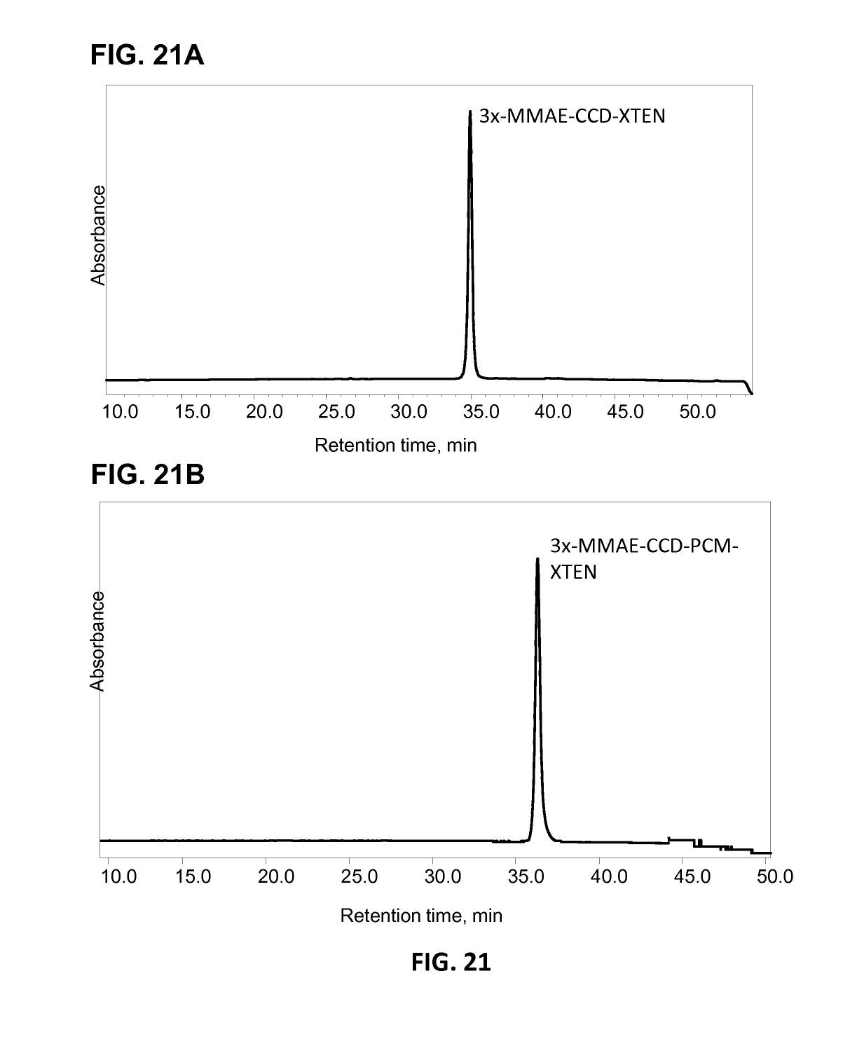

The mixture was acidified to pH <3 with TFA, and DMF was added to final volume of 13% (v/v). The desired 3x-MMAE-XTEN product was purified by preparative C4 RP-HPLC (C4, Vydac, 250 mm×22 mm). Chromatographic fractions were analyzed by C4 RP-HPLC. Fractions containing the desired product were pooled, neutralized with 1 M HEPES pH 8.0, and purified over a MacroCap Q column (GE Healthcare), with elution using a gradient of 10 column volumes from 150 mM to 350 mM NaCl. Chromatographic fractions were analyzed by C4 RP-HPLC. Selected fractions with 3x-MMAE-CCD-XTEN were pooled and formulated into 20 mM HEPES pH 7.0, 50 mM NaCl using ultrafiltration. High purity of the final product was demonstrated by C4 RP-HPLC (>95%, FIG. 21A). The reaction yield was determined to be 57.9%, which was superior to that achieved in the conjugation of IA-MMAE to the CXTEN (see Example 10, above).

Conclusions: The incorporation of CCD into the fusion proteins of the constructs resulted in an enhanced ability to recover the desired fully-conjugated product compared to constructs utilizing CXTEN (as determined by Peak Separation) and overall higher product yields.

### Example 12

### Conjugation of IA-MMAE to CCD-PCM-XTEN to Produce 3x-MMAE-CCD-PCM-XTEN

CCD-PCM-XTEN (XTEN_AE42(Am1,C8,C24,C40)-PCM-XTEN_AE713 was reduced with 3 molar equivalents of TCEP at pH 8.0 for 20 min at 80° C. The reduced CCD-PCM-XTEN was then reacted with 6 molar equivalents of IA-MMAE at pH 8.0 for 2 d at 25° C. The reaction was quenched with 30 molar equivalents of glutathione for 30 min at 25° C., and the conjugation was assessed by C4 RP-HPLC. The mixture was acidified to pH <3 with TFA, and DMF was added to final volume of 13% (v/v). The desired 3x-MMAE-CCD-PCM-XTEN product was purified by preparative C4 RP-HPLC (C4, Vydac, 250 mm×22 mm). Chromatographic fractions were analyzed by C4 RP-HPLC. Fractions containing the desired product were pooled, neutralized with 1 M HEPES pH 8.0, and purified over a MacroCap Q column (GE Healthcare), with elution using a gradient of 10 column volumes from 150 mM to 350 mM NaCl. Chromatographic fractions were analyzed by C4 RP-HPLC. Selected fractions with 3x-MMAE-CCD-PCM-XTEN were pooled and formulated into 20 mM HEPES pH 7.0, 50 mM NaCl using ultrafiltration. High purity of the final product was demonstrated by C4 RP-HPLC (>95%, FIG. 21B) and intact mass (observed ESI-MS of +12 Da).

### Example 13

### Preparation of Iodoacetamide-3x-DM1-CXTEN by Conjugation

The pH of 1.5 mL of XTEN_AE432(Am1,C12,C217,C422) (28.5 mg, 725 nmol) cysteine-engineered XTEN segment in 20 mM MES pH 5.5 was adjusted with 0.075 mL of 1 M HEPES pH 8.0. To this 3x-Cys-XTEN was added 0.067 mL of IA-DM1 (54 mM in anhydrous DMF), and additional DMF was added to fully solubilize the IA-DM1. The reaction was incubated at room temperature for 2.5 h and monitored by RP-HPLC. The 3x-DM1-CXTEN product was purified by preparative HPLC (C4, Vydac, 250 mm×10 mm). Chromatographic fractions were analyzed by C4 RP-HPLC. Fractions containing 3x-DM1-CXTEN were pooled, neutralized with 1 M HEPES pH 8.0, and concentrated under vacuum. The 3x-DM1-CXTEN product was formulated into 20 mM HEPES pH 7.0, 135 mM NaCl using ultrafiltration (Amicon Ultra-15, 3 kDa MWCO). The N-terminus of 3x-DM1-CXTEN was converted to the iodoacetamide by reaction of 0.45 mL of 3x-DM1-CXTEN (4.1 mg, 99 nmol) and 0.023 mL 1 M HEPES pH 8.0 with 0.01 mL of SIA (50 mM in anhydrous DMF) at room temperature for 2 h. Excess SIA was removed during formulation into 20 mM HEPES pH 7.0, 135 mM NaCl by ultrafiltration (Amicon Ultra-15, 5 kDa MWCO).

### Example 14

### Preparation of Iodoacetamide-3x-MMAE-CXTEN by Conjugation

The pH of 1 mL of XTEN_AE432(Am1,C12,C217,C422) (30.4 mg) cysteine-engineered XTEN segment in 20 mM MES pH 5.5 was adjusted with 0.27 mL of 1 M HEPES pH 8.0. To this 3x-Cys-XTEN was added IA-MMAE (5 molar equivalents, 64.6 mg/mL in anhydrous DMF), and additional 0.3 mL DMF was added to fully solubilize the IA-MMAE in the mixture. The reaction was incubated at 25° C. for 4 h and monitored by C4 RP-HPLC. The 3x-MMAE-CXTEN product was split into two aliquots, and each was purified by preparative HPLC (C4, Vydac, 250 mm×10 mm) Chromatographic fractions were analyzed by C4 RP-HPLC. Fractions containing 3x-MMAE-CXTEN were pooled, neutralized with 1 M HEPES pH 8.0, and concentrated under vacuum. The 3x-MMAE-CXTEN product was formulated into 20 mM HEPES pH 7.0, 50 mM NaCl using ultrafiltration (Amicon Ultra-15, 3 kDa MWCO). The N-terminus of 3x-MMAE-CXTEN was converted to the iodoacetamide by reaction of 3x-MMAE-CXTEN (126 mg, 2940 nmol) in 15 mL of 20 mM HEPES pH 7.0, 50 mM NaCl with 0.15 mL of SIA (10 molar equivalents, 200 mM in anhydrous DMF) at 25° C. for 1.5 h. Excess SIA was removed during formulation into 20 mM HEPES pH 7.0 by ultrafiltration (Sartorius, Vivacell 100, 5 kDa MWCO). The results of ESI-MS (calc MW 43,024.5 Da, obs MW 43,022.0 Da) and IA reactivity with model cysteine-containing peptide HCKFWW (Bachem, H-3524) of 87.4% demonstrated high purity and reactivity of the final product.

### Example 15

### Preparation of MCC-3x-MMAE-CCD-XTEN or MCC-3x-MMAE-CCD-PCM-XTEN

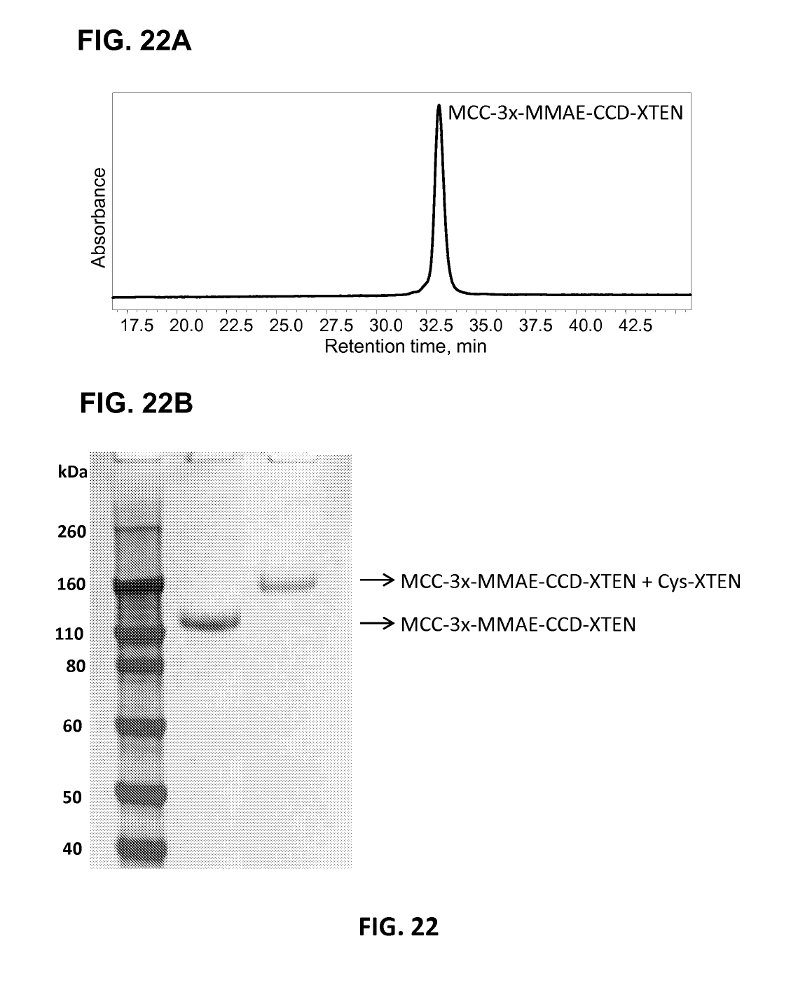

30 mg of 3x-MMAE-CCD-XTEN (from Example 11) or 3x-MMAE-CCD-PCM-XTEN (from Example 12) was reacted with 10 molar equivalents SMCC (dissolved in DMF) in 20 mM HEPES pH 7.0, 50 mM NaCl, 3.3% (v/v) DMF for 1 h at 25° C. temperature. Excess SMCC was removed by ultrafiltration using 20 mM HEPES pH 7.0, 50 mM NaCl. High purity of product was demonstrated by C4 RP-HPLC (>95%, FIG. 22A) and SDS-PAGE (FIG. 22B). MCC-3x-MMAE-CCD-XTEN and MCC-3x-MMAE-CCD-PCM-XTEN reactivity were each assessed by reaction with model cysteine-containing XTEN (XTEN_AE288(Am1,C283). The results of MCC reactivity showed high yield of maleimide products (FIG. 22B).

### Example 16

### Conjugation of aHER2-XTEN to 3x-DM1-CXTEN to Make an aHER2-Targeted XTEN-3x-DM1 Conjugate

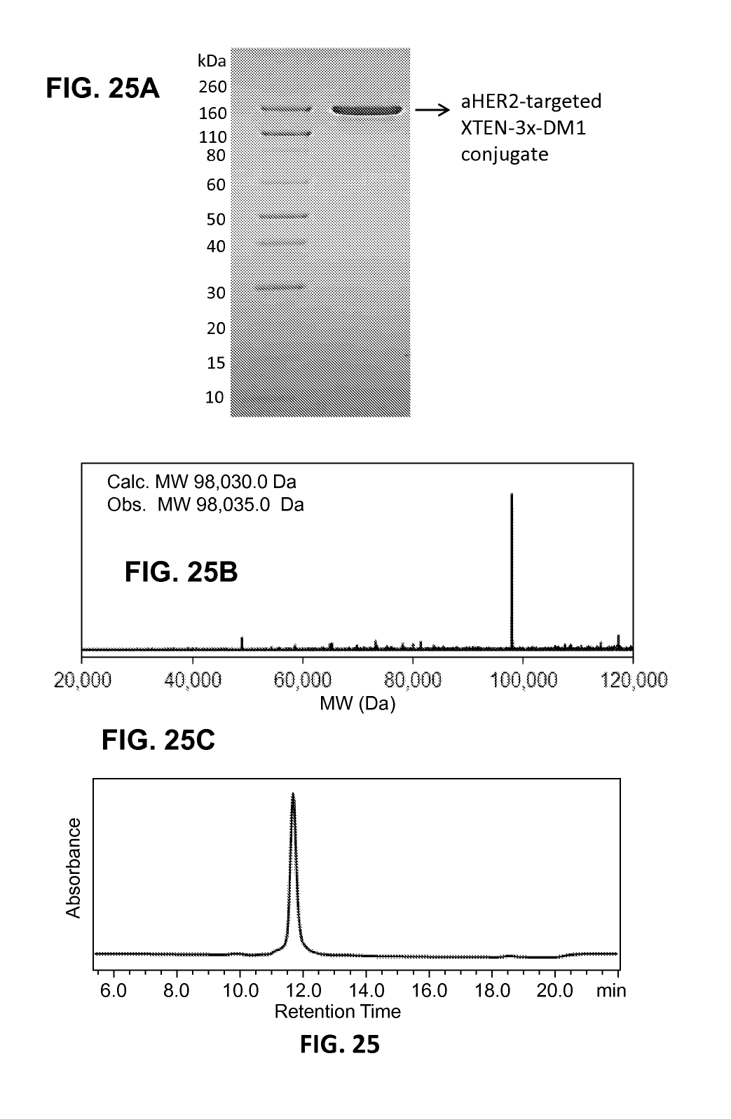

10 mL of aHER2-XTEN_AE304(C296)-H8 (153 mg, 2722 nmol) in 20 mM HEPES pH 7.0, 50 mM NaCl was reduced with 0.5 mM TCEP for 1.5 hat room temperature. Excess TCEP was removed by loading 2.5 mL each to four desalting columns (GE, PD-10) and eluting each with 3.5 mL of 20 mM HEPES pH 7.0, 50 mM NaCl (14 mL total). The cysteine side chain of reduced of aHER2-XTEN_AE304(C296)-H8 (153 mg, 2722 nmol) was reacted with the N-terminal iodoacetamide of IA-3xDM1-CXTEN (1853 nmol, see Example 13) in a total volume of 28 mL of 20 mM HEPES pH 7.0, 50 mM NaCl with pH adjusted to 8.5 with sodium borate buffer at 25° C. overnight. Reaction was monitored by SDS-PAGE. The mixture was loaded onto an immobilized metal affinity chromatography column (Toyopearl AF-Chelate 650M, 150 mL, 50 mm diameter) charged with Cu(II). Unreacted IA-3x-DM1-CXTEN was removed in the flow-through. aHER2-targeted XTEN-drug conjugate was eluted with 10 mM to 100 mM imidazole in 20 mM phosphate pH 7.0. Chromatographic fractions were analyzed by SDS-PAGE. Fractions with desired conjugate were pooled and purified over a MacroCap Q column (GE Healthcare, 100 mL, 50 mm diameter), with elution in 18 column volume gradient from 150 mM to 350 mM NaCl in 20 mM HEPES pH 7.0. Chromatographic fractions were analyzed by SDS-PAGE. Selected fractions with the aHER2-targeted CXTEN-3x-DM1 conjugate were pooled and formulated into PBS using ultrafiltration (Sartorius, Vivacell 100, 10 kDa MWCO). The results of SDS-PAGE (FIG. 25A), ESI-MS (FIG. 25B), and HIC (FIG. 25C) analyses demonstrated high purity of the fmal product.

TABLE 32
Amino acid sequence of components in Examples 16 and 17

| Component Name | Amino Acid Sequence |
|---|---|
| aHER2-XTEN_AE304 | DIQMTQSPSSLSASVGDRVTITCRASQDVNTAVAWYQQKPG |
| (C296)-118 | KAPKLLIYSASFLYSGVPSRFSGSRSGTDFTLTISSLQPEDFAT |
| | YYCQQHYTTPPTFGQGTKVEIKTGSGEGSEGEGGGEGSEGE |
| | GSGEGGEGEGSGTEVQLVESGGGLVQPGGSLRLSCAASGFN |
| | IKDTYIHWVRQAPGKGLEWVARIYPTNGYTRYADSVKGRF |
| | TISADTSKNTAYLQMNSLRAEDTAVYYCSRWGGDGFYAM |
| | DYWGQGTLVTVSGSPGTSESATPESGPGSEPATSGSETPGTS |
| | ESATPESGPGSEPATSGSETPGTSESATPESGPGTSTEPSEGSA |
| | PGSPAGSPTSTEEGTSESATPESGPGSEPATSGSETPGTSESAT |
| | PESGPGSPAGSPTSTEEGSPAGSPTSTEEGTSTEPSEGSAPGTS |
| | ESATPESGPGTSESATPESGPGTSESATPESGPGSEPATSGSET |
| | PGSEPATSGSETPGSPAGSPTSTEEGTSTEPSEGSAPGTSTEPS |
| | EGSAPGSEPATSGSETPGTSESATPESGPGTSTEPSEGSAPGG |
| | APCGPAGGSSSHHHHHHHH (SEQ ID NO 723) |
| XTEN_AE432 (Am1, C12, | SAGSPTAEAAGCGTAEAAGTSESATPESGPGSEPATSGSETP |
| C217, C422) | GTSESATPESGPGTSTEPSEGSAPGTSESATPESGPGSPAGSPT |
| | STEEGSPAGSPTSTEEGSPAGSPTSTEEGTSESATPESGPGTST |
| | EPSEGSAPGTSESATPESGPGSEPATSGSETPGTSESATPESGP |
| | GSEPATSGSETPGTSESATPESGPGTSTEPSEGSAPTAEAAGC |
| | GTAEAAGSPAGSPTSTEEGTSESATPESGPGSEPATSGSETPG |
| | TSESATPESGPGSPAGSPTSTEEGSPAGSPTSTEEGTSTEPSEG |
| | SAPGTSESATPESGPGTSESATPESGPGTSESATPESGPGSEPA |
| | TSGSETPGSEPATSGSETPGSPAGSPTSTEEGTSTEPSEGSAPG |
| | TSTEPSEGSAPGSEPATSGSETPTAEAAGCGTAEAASASR |
| | (SEQ ID NO 724) |

### Example 17

### Conjugation of aHER2-XTEN to 3x-MMAE-CXTEN to Make an aHER2-Targeted XTEN-3x-MMAE Conjugate

10 mL of aHER2-XTEN_AE304(C296)-H8 (153 mg, 2722 nmol) in 20 mM HEPES pH 7.0, 50 mM NaCl was reduced with 0.5 mM TCEP for 1.5 hat room temperature. Excess TCEP was removed by loading 2.5 mL each to four desalting columns (GE, PD-10) and eluting each with 3.5 mL of 20 mM HEPES pH 7.0, 50 mM NaCl (14 mL total). The cysteine side chain of reduced of aHER2-XTEN_AE304(C296)-H8 (153 mg, 2722 nmol) was reacted with the N-terminal iodoacetamide of IA-3xMMAE-CXTEN (2722 nmol, see Example 14) in a total volume of 20 mL of 20 mM HEPES pH 7.0, 50 mM NaCl with pH adjusted to 8.5 with sodium borate buffer at 25° C. overnight. Reaction was monitored by SDS-PAGE. The mixture was loaded onto an immobilized metal affinity chromatography column (Toyopearl AF-Chelate 650M, 150 mL, 50 mm diameter) charged with Cu(II). Unreacted IA-3x-MMAE-XTEN was removed in the flow-through. aHER2-targeted XTEN-drug conjugate was eluted with 10 mM to 100 mM imidazole in 20 mM phosphate pH 7.0. Chromatographic fractions were analyzed by SDS-PAGE. Fractions with desired conjugate were pooled and purified over a MacroCap Q column (GE Healthcare, 100 mL, 50 mm diameter), with elution in 14 column volume gradient from 150 mM to 350 mM NaCl in 20 mM HEPES pH 7.0. Chromatographic fractions were analyzed by SDS-PAGE. Selected fractions with the aHER2-targeted XTEN-3x-MMAE conjugate were pooled and formulated into PBS using ultrafiltration (Sartorius, Vivacell 100, 10 kDa MWCO). The results of SDS-PAGE (FIG. 29A) and ESI-MS (FIG. 29B) analyses demonstrated high purity of the final product.

### Example 18

### Conjugation of aHER2-XTEN to MCC-3x-MMAE-CCD-XTEN to Make an aHER2-Targeted CCD-XTEN-Drug Conjugate

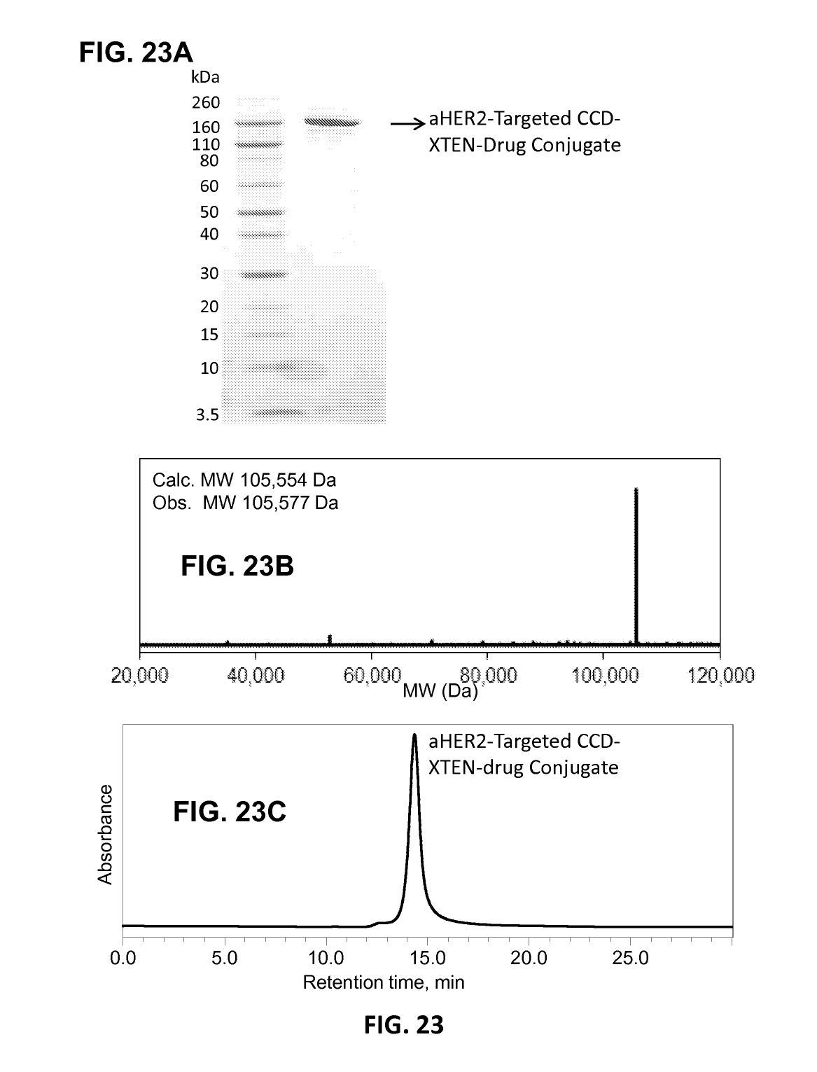

aHER2-XTEN_AE44(C36)-H8 (10.2 mg, 315 nmol) in 1 mL of 20 mM HEPES pH 7.0, 50 mM NaCl was reduced with 1 mM TCEP for 1.5 hat room temperature. Excess TCEP was removed by desalting column (GE, PD MiniTrap G-25), eluting in 1.5 mL of 20 mM HEPES pH 7.0, 50 mM NaCl. The cysteine side chain of reduced aHER2-XTEN_AE44(C36)-H8 (10.2 mg, 315 nmol) was reacted with the N-terminal maleimide of MCC-3x-MMAE-CCD-XTEN (13 mg, 178 nmol, see Example 15) in a total volume of 2.5 mL 20 mM HEPES pH 7.0, 50 mM NaCl at 25° C. for 2 h. The reaction was monitored by SDS-PAGE. The reaction mixture was diluted with 6.5 mL of 20 mM sodium phosphate pH 7.0 and loaded onto an immobilized metal affinity chromatography column (Toyopearl AF-Chelate 650M, 9 mL, 15 mm diameter) charged with Cu(II). Unreacted MCC-3x-MMAE-CCD-XTEN was removed in the flow-through. aHER2-targeted XTEN-drug conjugate was eluted with 10 mM to 100 mM imidazole in 20 mM phosphate pH 7.0. Chromatographic fractions were analyzed by SDS-PAGE. Fractions with desired conjugate were pooled and purified over a MacroCap Q column (GE Healthcare, 10 mL, 16 mm diameter), with elution using a 10 column volume gradient from 150 mM to 350 mM NaCl in 20 mM HEPES pH 7.0. Chromatographic fractions were analyzed by SDS-PAGE. Selected fractions with aHER2-targeted XTEN-drug conjugate were pooled and formulated into PBS using ultrafiltration (Sartorius, Vivaspin 15R, 5 kDa MWCO). The results of SDS-PAGE (FIG. 23A), ESI-MS (FIG. 23B), and SEC-HPLC (FIG. 23C) analyses demonstrated high purity of the final product.

TABLE 33
Amino acid sequence of components in Example 18

| Component Name | Amino Acid SequenCe |
|---|---|
| aHER2-XTEN_AE44 | DIQMTQSPSSLSASVGDRVTITCRASQDVNTAVAWYQQKPG |
| (C36)-118 | KAPKLLIYSASFLYSGVPSRFSGSRSGTDFTLTISSLQPEDFAT |
| | YYCQQHYTTPPTFGQGTKVEIKTGSGEGSEGEGGGEGSEGE |
| | GSGEGGEGEGSGTEVQLVESGGGLVQPGGSLRLSCAASGFN |
| | IKDTYIHWVRQAPGKGLEWVARIYPTNGYTRYADSVKGRF |
| | TISADTSKNTAYLQMNSLRAEDTAVYYCSRWGGDGFYAM |
| | DYWGQGTLVTVSGSPGSAGSGSETPGTSESATPESGPGTSTE |
| | PSGAPCGPAGGSSSHHHHHHHH (SEQ ID NO 725) |
| XTEN_AE759 (Am1, | GSPGAGSCAGSPTSTEEGTSESACSPEGPGTSTEPSEGSCGGP |
| C8, C24, C40) | GSPAGSPTSTEEGTSESATPESGPGTSTEPSEGSAPGSPAGSPT |
| | STEEGTSTEPSEGSAPGTSTEPSEGSAPGTSESATPESGPGSEP |
| | ATSGSETPGSEPATSGSETPGSPAGSPTSTEEGTSESATPESGP |
| | GTSTEPSEGSAPGTSTEPSEGSAPGSPAGSPTSTEEGTSTEPSE |
| | GSAPGTSTEPSEGSAPGTSESATPESGPGTSTEPSEGSAPGTSE |
| | SATPESGPGSEPATSGSETPGTSTEPSEGSAPGTSTEPSEGSAP |
| | GTSESATPESGPGTSESATPESGPGSPAGSPTSTEEGTSESATP |
| | ESGPGSEPATSGSETPGTSESATPESGPGTSTEPSEGSAPGTST |
| | EPSEGSAPGTSTEPSEGSAPGTSTEPSEGSAPGTSTEPSEGSAP |
| | GTSTEPSEGSAPGSPAGSPTSTEEGTSTEPSEGSAPGTSESATP |
| | ESGPGSEPATSGSETPGTSESATPESGPGSEPATSGSETPGTSE |
| | SATPESGPGTSTEPSEGSAPGTSESATPESGPGSPAGSPTSTEE |
| | GSPAGSPTSTEEGSPAGSPTSTEEGTSESATPESGPGTSTEPSE |
| | GSAPGTSESATPESGPGSEPATSGSETPGTSESATPESGPGSEP |
| | ATSGSETPGTSESATPESGPGTSTEPSEGSAPGSPAGSPTSTEE |
| | GTSESATPESGPGSEPATSGSETPGTSESATPESGPGSPAGSPT |
| | STEEGSPASASR (SEQ ID NO: 726) |

### Example 19

### Conjugation of aHER2-XTEN to MCC-3x-MMAE-CCD-PCM-XTEN to Make an aHER2-Targeted CCD-XTEN-Drug Conjugate with PCM

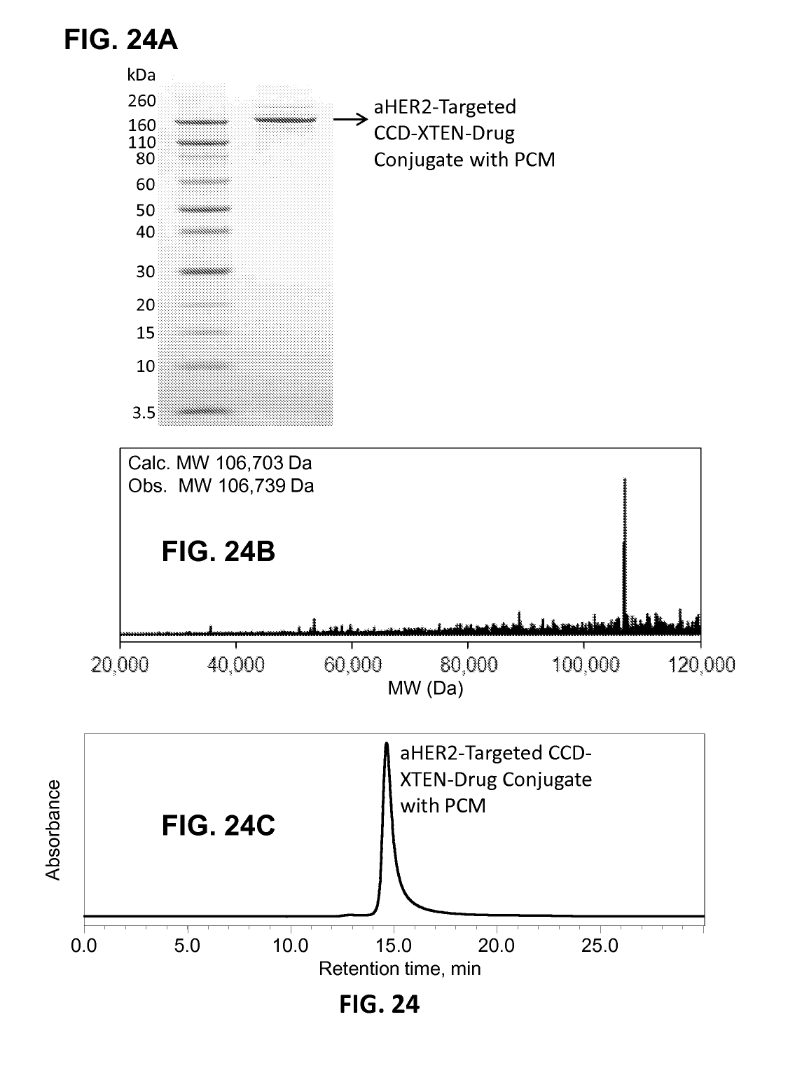

aHER2-XTEN_AE44(C36)-H8 (20.4 mg, 630 nmol) in 2 mL of 20 mM HEPES pH 7.0, 50 mM NaCl was reduced with 0.75 mM TCEP for 1.5 h at room temperature. Excess TCEP was removed by desalting column (GE, PD-10), eluting in 3.5 mL of 20 mM HEPES pH 7.0, 50 mM NaCl. The cysteine side chain of reduced aHER2-XTEN_AE44(C36)-H8 (20.4 mg, 630 nmol) was reacted with the N-terminal maleimide of MCC-3x-MMAE-CCD-PCM-XTEN (30 mg, 403 nmol, see Example 15) in a total volume of 7.3 mL 20 mM HEPES pH 7.0, 50 mM NaCl at 25° C. for 2 h. The reaction was monitored by SDS-PAGE. The mixture was diluted with 12.7 mL of 20 mM sodium phosphate pH 7.0 and loaded onto an immobilized metal affinity chromatography column (Toyopearl AF-Chelate 650M, 20 mL, 27 mm diameter) charged with Cu(II). Unreacted MCC-3x-MMAE-CCD-PCM-XTEN was removed in the flow-through. aHER2-targeted XTEN-drug conjugate was eluted with 10 mM to 100 mM imidazole in 20 mM phosphate pH 7.0. Chromatographic fractions were analyzed by SDS-PAGE. Fractions with desired conjugate were pooled and purified over a MacroCap Q column (GE Healthcare, 25 mL, 16 mm diameter), with elution using a 10 column volume gradient from 150 mM to 350 mM NaCl in 20 mM HEPES pH 7.0. Chromatographic fractions were analyzed by SDS-PAGE. Selected fractions with pure aHER2-targeted XTEN-drug conjugate with PCM were pooled and formulated into PBS using ultrafiltration (Sartorius, Vivaspin 15R, 5 kDa MWCO). The results of SDS-PAGE (FIG. 24A), ESI-MS (FIG. 24B), and SEC-HPLC (FIG. 24C) analyses demonstrated high purity of the final product.

TABLE 34
Amino acid sequence of components in Example 19

| Component Name | Amino Acid Sequence |
|---|---|
| aHER2-XTEN_AE44 | DIQMTQSPSSLSASVGDRVTITCRASQDVNTAVAWYQQKPG |
| (C36)-118 | KAPKLLIYSASFLYSGVPSRFSGSRSGTDFTLTISSLQPEDFAT |
| | YYCQQHYTTPPTFGQGTKVEIKTGSGEGSEGEGGGEGSEGE |
| | GSGEGGEGEGSGTEVQLVESGGGLVQPGGSLRLSCAASGFN |
| | IKDTYIHWVRQAPGKGLEWVARIYPTNGYTRYADSVKGRF |
| | TISADTSKNTAYLQMNSLRAEDTAVYYCSRWGGDGFYAM |
| | DYWGQGTLVTVSGSPGSAGSGSETPGTSESATPESGPGTSTE |
| | PSGAPCGPAGGSSSHHHHHHHH (SEQ ID NO: 727) |
| XTEN_AE42 (Am1, C8, | GSPGAGSCAGSPTSTEEGTSESACSPEGPGTSTEPSEGSCGG |
| C24, C40) | (SEQ ID NO: 728) |
| PCM | LSGRSDNHSPLGLAGS (SEQ ID NO: 729) |
| XTEN_AE713 | PGSPAGSPTSTEEGTSESATPESGPGTSTEPSEGSAPGSPAGSP |
| | TSTEEGTSTEPSEGSAPGTSTEPSEGSAPGTSESATPESGPGSE |
| | PATSGSETPGSEPATSGSETPGSPAGSPTSTEEGTSESATPESG |
| | PGTSTEPSEGSAPGTSTEPSEGSAPGSPAGSPTSTEEGTSTEPS |
| | EGSAPGTSTEPSEGSAPGTSESATPESGPGTSTEPSEGSAPGTS |
| | ESATPESGPGSEPATSGSETPGTSTEPSEGSAPGTSTEPSEGSA |
| | PGTSESATPESGPGTSESATPESGPGSPAGSPTSTEEGTSESAT |
| | PESGPGSEPATSGSETPGTSESATPESGPGTSTEPSEGSAPGTS |
| | TEPSEGSAPGTSTEPSEGSAPGTSTEPSEGSAPGTSTEPSEGSA |
| | PGTSTEPSEGSAPGSPAGSPTSTEEGTSTEPSEGSAPGTSESAT |
| | PESGPGSEPATSGSETPGTSESATPESGPGSEPATSGSETPGTS |
| | ESATPESGPGTSTEPSEGSAPGTSESATPESGPGSPAGSPTSTE |
| | EGSPAGSPTSTEEGSPAGSPTSTEEGTSESATPESGPGTSTEPS |
| | EGSAPGTSESATPESGPGSEPATSGSETPGTSESATPESGPGSE |
| | PATSGSETPGTSESATPESGPGTSTEPSEGSAPGSPAGSPTSTE |
| | EGTSESATPESGPGSEPATSGSETPGTSESATPESGPGSPAGSP |
| | TSTEEGSPA (SEQ ID NO: 730) |

### Example 20

### Conjugation of Folate-AHHAC to IA-3x-MMAE-CCD-XTEN to Make a Folate-Targeted CCD-XTEN-Drug Conjugate

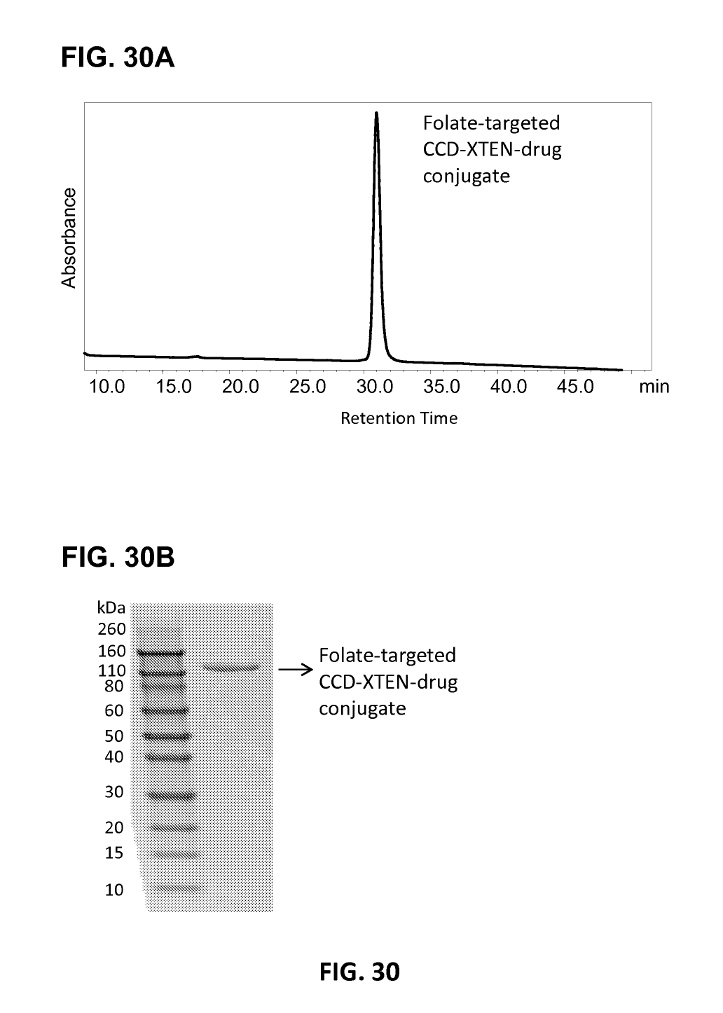

The N-terminus of 3x-MMAE-CCD-XTEN (18.1 mg, see Example 11) was converted to iodoacetamide with SIA (10 molar equivalents) reacted with the cysteine side chain of Folate-AHHAC (3 molar equivalents) in a total volume of 1.8 mL 200 mM HEPES pH 7.0, 50 mM NaCl at 25° C. overnight. The reaction mixture was loaded onto an immobilized metal affinity chromatography column (Toyopearl AF-Chelate 650M, 20 mL, 27 mm diameter) charged with Cu(II). Unreacted IA-3x-MMAE-CCD-XTEN was removed in the flow-through. Folate-targeted XTEN-drug conjugate was eluted with 50 mM imidazole in 20 mM phosphate pH 8.0. Chromatographic fractions were analyzed by MALDI-MS and SDS-PAGE. Fractions with desired conjugate were pooled, acidified to pH <3 with TFA, then purified by preparative HPLC (C4, Vydac, 250 mm×10 mm) Chromatographic fractions were analyzed by MALDI and RP-HPLC. Fractions containing the folate-targeted XDC were pooled, neutralized with 1 M HEPES pH 8.0, and concentrated under vacuum. The product was formulated into PBS using ultrafiltration (Amicon Ultra-4, 3 kDa MWCO). Purity of the product was demonstrated by C4 RP-HPLC (FIG. 30A) and SDS-PAGE (FIG. 30B).

### Example 21

### Conjugation of Folate-AHHAC to MCC-3x-MMAE-CCD-PCM-XTEN to Make a Folate-Targeted CCD-XTEN-Drug Conjugate with PCM

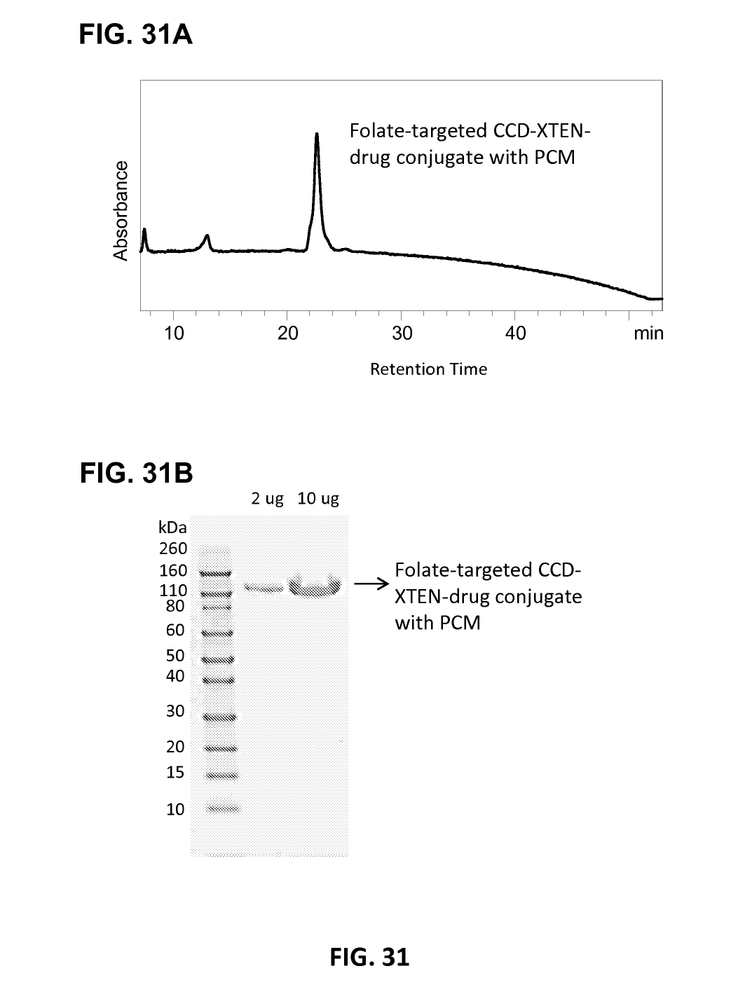

MCC-3x-MMAE-CCD-PCM-XTEN (33.5 mg, see Example 15) was reacted with the cysteine side chain of Folate-AHHAC (3 molar equivalents) in a total volume of 4.3 mL 200 mM HEPES pH 7.0, 50 mM NaCl at 25° C. overnight. The reaction mixture was loaded onto an immobilized metal affinity chromatography column (Toyopearl AF-Chelate 650M, 20 mL, 27 mm diameter) charged with Cu(II). Unreacted MCC-3x-MMAE-CCD-PCM-XTEN was removed in the flow-through. Folate-targeted XTEN-drug conjugate with PCM was eluted with 50 mM imidazole in 20 mM phosphate pH 8.0. Chromatographic fractions were analyzed by MALDI-MS. Fractions with desired conjugate were pooled, acidified to pH <3 with TFA, then purified by preparative HPLC (C4, Vydac, 250 mm×10 mm). Chromatographic fractions were analyzed by MALDI-MS and RP-HPLC. Fractions containing the folate-targeted XDC were pooled, neutralized with 1 M HEPES pH 8.0, and concentrated under vacuum. The product was formulated into PBS using ultrafiltration (Amicon Ultra-15, 3 kDa MWCO). Purity of the product was demonstrated by C4 RP-HPLC (FIG. 31A) and SDS-PAGE (FIG. 31B).

### Example 22

### Preparation of A Bispecific Conjugate from Monospecific XTEN Precursors Linked by the N-termini

The example describes the creation of an XTEN-conjugate composition by linking two different XTEN-payload precursors in an N- to N-terminus configuration; one with a payload A and one with a payload B, resulting in a bispecific conjugate.

As a first step, XTEN molecules containing multiple cysteines (cysteine-engineered XTEN) are prepared as described above, and are formulated in 20 mM HEPES, pH 7.0, 50 mM NaCl. A Payload A-maleimide is dissolved in aqueous solution 20 mM HEPES, pH 7.0, DMF or DMCO or any other appropriate solvent depending on reagent solubility. The Payload A-maleimide is added to the cysteine-engineered XTEN in a 2-6 molar excess over XTEN and incubated for 1 hr at 25° C. Completion of modification is monitored by C18 RP-HPLC. The resulting Payload A-XTEN conjugate is purified from contaminants and unreacted components using preparative C4-C18 RP-HPLC. The Payload A-XTEN conjugate is formulated in 20 mM HEPES, pH 7.0, 50 mM NaCl. Next, the Payload A-XTEN conjugate is further modified by adding dibenzylcyclooctyne (DBCO)-NHS ester or DBCO-sulfo-NHS ester in a 10-50 molar excess to the XTEN and incubating 1-2 hrs at 25° C. Completion of the modification is monitored by analytical C18 RP-HPLC. If the conjugation efficiency is low (for example, <90%) or multiple unspecific products are formed, the DBCO-Payload A-XTEN conjugate is purified using preparative C4-C18 RP-HPLC. If the efficiency of DBCO-NHS ester conjugation is high (>90%) with no significant side products, the DBCO-Payload A-XTEN conjugate is purified from excess reagent by buffer exchange using a 10-30 kDa MWCO centrifugal device, acetonitrile precipitation or anion exchange chromatography.

To create the second XTEN-payload precursor, a Payload B-maleimide is dissolved in aqueous solution 20 mM HEPES, pH 7.0, DMF or DMCO or any other appropriate solvent depending on reagent solubility. Payload B-maleimide is added to the second cysteine-containing XTEN in 2-6 molar excess over XTEN concentration and incubated for 1 hr at 25° C. Completion of modification is monitored by analytical C18 RP-HPLC. The resulting Payload B-XTEN conjugate is purified from contaminants and reactants using preparative C4-C18 RP-HPLC. The Payload B-XTEN conjugate is formulated in 20 mM HEPES, pH 7.0, 50 mM NaCl. Azide-PEG4-NHS ester is added in 10-50 molar excess to the Payload B-XTEN and incubated 1-2 hrs at 25° C. Completion of modification is monitored by C18 RP-HPLC. If the conjugation efficiency is low (for example <90%) or multiple unspecific products are formed, the azide-Payload B-XTEN conjugate is purified using preparative C4-C18 RP-HPLC. If the efficiency of DBCO-NHS ester conjugation is high (>90%) with no significant side products, the azide-Payload B-XTEN conjugate is purified from excess reagent by buffer exchange using a 10-30 kDa MWCO centrifugal device, acetonitrile precipitation or anion exchange chromatography. The final product is created by mixing purified and concentrated DBCO-Payload A-XTEN and azide-Payload B-XTEN proteins in an equilmolar ratio in 20 mM HEPES pH 7.0 buffer, 50 mM NaCl and incubated at 25° C. for 1 hr or longer until the reaction is complete. Completion of modification is monitored by C4 or C18 RP-HPLC. If necessary, the bispecific conjugate Payload A-XTEN-Payload B is purified by preparatiove RP-HPLC, hydrophobic interaction chromatography or anion exchange chromatography.

### Example 23

### Preparation of A Trimeric Conjugate from Monospecific XTEN Precursors Linked by the N-termini

Monospecific XTEN-payload precursors will be prepared as N-terminal fusions of a Payload A linked to an XTEN molecule; e.g. of lengths ranging from AE144 to AE890, containing a single cysteine at the C-terminus. Purified precursors are formulated in 20 mM HEPES, pH 7.0, 50 mM NaCl. Tris-(2-maleimidoethyl)amine (TMEA, Thermo Scientific, cat. #33043) and dissolved in DMSO or DMF. Precursor (4-6 molar excess over cross-linker) and TMEA reagent are mixed and incubated for 1 hr at 25° C. Completion of the modification is monitored by C4 or C18 RP-HPLC or size exclusion chromatography. The resulting trivalent Payload A-XTEN conjugate is purified from protein reactants or partial product mixture by hydrophobic interaction chromatography (HIC), anion exchange chromatography or preparative C4-C18 RP-HPLC.

### Example 24

### In Vitro Serum Stability of Folate-XTEN-Drug Conjugates

As a measure of stability, folate-XTEN-drug conjugates are incubated independently in normal human, cynomolgus monkey and mouse plasma at 37° C. for up to 2 weeks with aliquots removed at periodic intervals and stored at −80° C. until analysis. The stability of folate-XTEN-drug conjugate is assessed either by the amount of free drug or the integrity of the folate-XTEN-drug conjugate over time. Free drug is quantitated with HPLC and/or LC-MS/MS whereas the amount of intact folate-XTEN-drug conjugate is determined using an XTEN/drug and/or folate/drug ELISA.

For RP-HPLC analysis, plasma samples are treated with organic solvents such as acetonitrile or acetone to precipitate proteins. Soluble fractions are evaporated under vacuum, redissolved in loading solutions and analyzed by RP-HPLC. Analytes are detected by UV absorption at wavelength specific for a particular drug, compared to known drug standards. For example, doxorubicin is detected at 480 nm. For LC-MS/MS analysis, plasma samples will be treated with organic solvents such as acetonitrile or acetone to precipitate proteins. Soluble fractions will be evaporated in vacuum, redissolved in loading solutions and analyzed by RP-HPLC. Analytes will be in-line detected and quantitated by triple quadrupole tandem mass spectrometry. Parental ion-daughter ion pairs will be determined experimentally for each drug. Calibration standards will be prepared by adding known amounts of free drug to corresponding plasma type and will be treated in parallel with experimental samples. For quantitative ELISA, optimal concentrations of antibodies for folate-XTEN-drug conjugate in the ELISAs is determined using criss-cross serial dilution analysis. An appropriate capture antibody recognizing one component of the conjugate is coated onto a 96-well microtiter plate by an overnight incubation at 4° C. The wells are blocked, washed and serum stability samples added to the wells, each at varying dilutions to allow optimal capture of the folate-XTEN-drug conjugate by the coated antibody. After washing, detection antibody recognizing another component of the conjugate is added and allowed to bind to the conjugate captured on the plate. Wells are then washed again and either streptavidin-horseradish peroxidase (complementary to biotinylated version of detection antibody) or an appropriate secondary antibody-horseradish peroxidase (complementary to non-biotinylated version of detection antibody) is then added. After appropriate incubation and a final wash step, tetramethylbenzidine (TMB) substrate is added and the plate is read at 450 nM. Concentrations of intact conjugate are then calculated for each time point by comparing the colorimetric response to a calibration curve prepared with folate-XTEN-drug in the relevant plasma type. The t1/2 of the decay of the conjugate in human, cyno and mouse serum is then defined using linear regression analysis of the log concentrations vs. time.

### Example 25

### Preparation of aHER2-XTEN-Alexa Fluor 568 Conjugate

1x-amino,1x-thiol-XTEN (XTEN_AE864(Am1,C12), 124 nmol) in 0.5 mL of 20 mM MES pH 5.5 was neutralized with 20 uL of 1 M HEPES pH 8. The thiol of this XTEN was then reacted with 3 molar equivalents of Alexa Fluor 568 C5 Maleimide (Thermo Fisher Scientific, catalog #A20341, 372 nmol, 0.0372 mL of a 10 mM solution in anhydrous DMF). The reaction was incubated at room temperature for 2 h, and the conjugation was monitored by C18 RP-HPLC. The mixture was acidified to pH <3 with TFA, and the desired XTEN-Alexa Fluor 568 product was purified by preparative C18 RP-HPLC (C18, Phenomenex, catalog #00G-4005-NO, 250 mm x 10 mm) Chromatographic fractions were analyzed by C18 RP-HPLC. Fractions containing the desired product were pooled, neutralized with 1 M HEPES pH 8.0, and concentrated under vacuum. Selected fractions with XTEN-Alexa Fluor 568 were pooled and formulated into 20 mM HEPES pH 7.0, 50 mM NaCl using ultrafiltration (Sartorius, Vivaspin 15R, 5 kDa MWCO). High purity of the fmal product was demonstrated by C18 RP-HPLC (>98%), SD S-PAGE, and intact mass (observed ESI-MS of +13 Da). The N-terminal amine of XTEN-Alexa Fluor 568 (51.7 nmol) in 0.4 mL of 20 mM HEPES pH 7.0, 50 mM NaCl was converted to an iodoacetamide using 10 molar equivalents of SIA (517 nmol, 49 mg/mL in anhydrous DMF) at room temperature in the dark for 2 h. Excess SIA was removed by ultrafiltration (Sartorius, Vivaspin 15R, 5 kDa MWCO).

aHER2-XTEN_AE44(C36)-H8 (2 mg, 63 nmol) in 0.2 mL of 20 mM HEPES pH 7.0, 50 mM NaCl was reduced with 0.25 mM TCEP for 1.5 h at room temperature. Excess TCEP was removed by desalting column (GE, PD MiniTrap G-25), eluting in 1.5 mL of 20 mM HEPES pH 7.0, 50 mM NaCl. This reduced aHER2-XTEN was concentrated by ultrafiltration (Sartorius, Vivaspin 500, 5 kDa MWCO) to 0.09 mL in 20 mM HEPES pH 7.0, 50 mM NaCl. The cysteine side chain of reduced aHER2-XTEN_AE44(C36)-H8 (2 mg, 64 nmol) was reacted with the N-terminal iodoacetamide of IA-XTEN-Alexa Fluor 568 (51.7 nmol, 0.26 mL in 20 mM HEPES pH 7.0, 50 mM NaCl) at approximately pH 9 by addition of 0.0055 mL of 0.4 M sodium borate pH 9.95. The reaction was incubated overnight at 25° C. then checked by SDS-PAGE. The reaction mixture was diluted to 3 mL with 20 mM sodium phosphate pH 8.0 and loaded onto an immobilized metal affinity chromatography column (Toyopearl AF-Chelate 650M, 3 mL, 15 mm diameter) charged with Cu(II). Unreacted IA-XTEN-Alexa Fluor 568 was removed in the flow-through. aHER2-targeted XTEN-fluorophore conjugate was eluted with 25 mM to 100 mM imidazole in 20 mM phosphate pH 8.0. Chromatographic fractions were analyzed by SDS-PAGE. Fractions with desired conjugate were pooled and purified over a MacroCap Q column (GE Healthcare, 10 mL, 16 mm diameter), with elution using a 10 column volume gradient from 150 mM to 350 mM NaCl in 20 mM HEPES pH 7.0. Chromatographic fractions were analyzed by SDS-PAGE. Selected fractions with aHER2-targeted XTEN-fluorophore conjugate were pooled and formulated into PBS using ultrafiltration (Sartorius, Vivaspin 15R, 5 kDa MWCO). The results of SDS-PAGE and intact mass (observed ESI-MS of +16 Da) demonstrated high purity of the final product.

### Example 26

### In Vivo and Ex Vivo Imaging of aHER2-XTEN-Alexa Fluor 568 Conjugate

An Alexa Fluor 568-tagged aHER2-XTEN molecule is used as a surrogate to investigate the targeting and biodistribution efficiency of aHER2-XTEN-drug conjugates. Experiments will be carried out in nude mice bearing subcutaneous grown xenografts of HER2 positive tumor cells using in vivo, followed by ex vivo, fluorescence imaging with IVIS 50 optical imaging system (Caliper Life Sciences, Hopkinton, Mass.). In brief, female nu/nu mice bearing HER2 positive tumor cells are given a single intravenous injection of high or low dose of aHER2-XTEN-Alexa Fluor 568 and corresponding doses of non-targeting Alexa Fluor 568-tagged XTEN control. Whole body scans are acquired pre-injection and then at approximately 8, 24, 48 and 72 hours post-injection on live anesthetized animals using the IVIS 50 optical imaging system. After measuring the distribution of fluorescence in the entire animal at the last time point of 72 h, tumor and healthy organs including liver, lung, heart, spleen and kidneys are excised and their fluorescence registered and processed by the imaging system. Small and medium binning of the CCD chip is used and the exposure time optimized to obtain at least several thousand counts from the signals that were observable in each mouse in the image and to avoid saturation of the CCD chip. To normalize images for quantification, a background fluorescence image is acquired using background excitation and emission filters for the Alexa Fluor 568 spectral region. The intensity of fluorescence is expressed as different colors with blue color reflecting the lowest intensity and red being indicative of the highest intensity, and the resulting images are used to assess the uptake of the conjugates and controls.

### Example 27

### In Vivo and Ex Vivo Imaging of Folate-XTEN-Cy5.5 Conjugates

A Cy5.5 fluorescent tagged folate-XTEN molecule is used as a surrogate to investigate the targeting and biodistribution efficiency of folate-XTEN-drug conjugates. Experiments will be carried out in nude mice bearing subcutaneous grown xenografts of folate receptor positive tumor cells using in vivo, followed by ex vivo, fluorescence imaging with IVIS 50 optical imaging system (Caliper Life Sciences, Hopkinton, Mass.). As culture media contain high folate content, folate receptor positive tumor cells to be transplanted onto these mice will be grown in folate-free cell culture media containing 5-10% heat-inactivated FCS with no antibiotics. Similarly, normal rodent chow contains a high concentration of folic acid; nude mice used in this study will be maintained on folate-free diet 2 weeks prior to tumor implantation and for the duration of the imaging analysis to reduce serum folate concentration.

In brief, female nu/nu mice bearing folate receptor positive tumor cells are given a single intravenous injection of high or low dose folate-XTEN-Cy5.5 and corresponding doses of non-targeting Cy5.5 tagged XTEN control. Whole body scans are acquired pre-injection and then at approximately 8, 24, 48 and 72 hours post-injection on live anesthetized animals using the IVIS 50 optical imaging system. After measuring the distribution of fluorescence in the entire animal at the last time point of 72 h, tumor and healthy organs including liver, lung, heart, spleen and kidneys are excised and their fluorescence registered and processed by the imaging system. Cy5.5 excitation (615-665 nm) and emission (695-770 nm) filters are selected to match the fluorescence agents' wavelengths. Small and medium binning of the CCD chip is used and the exposure time optimized to obtain at least several thousand counts from the signals that were observable in each mouse in the image and to avoid saturation of the CCD chip. To normalize images for quantification, a background fluorescence image is acquired using background excitation and emission filters for the Cy5.5 spectral region. The intensity of fluorescence is expressed as different colors with blue color reflecting the lowest intensity and red being indicative of the highest intensity, and the resulting images are used to assess the uptake of the conjugates and controls.

### Example 28

### Human Clinical Trial Designs for Evaluating Folate-XTEN-Drug Conjugates

Targeted chemotherapy is a modern approach aimed at increasing the efficacy of systemic chemotherapy and reducing its side effects. Folate, also known as folic acid, vitamin B₉, is a vital nutrient required by all living cells for nucleotide biosynthesis and function as cofactor in certain biological pathways. The folate receptor is a focus for the development of therapies to treat fast dividing malignancies; in particular ovarian cancer and non-small cell lung carcinoma. While folate receptor expression is negligible in normal ovary, ˜90% of epithelial ovarian cancers overexpress the folate receptor, as do many lung adenocarinomas, thereby opening the possibility of directed therapies. Fusion of a XTEN carrying ≥1 copy of folate to a XTEN bearing ≥3 drug molecules to create a targeted peptide-drug conjugate is expected to improve the therapeutic index and the extended half-life will enable dosing at levels way below maximum tolerated dose (MTD), reduce dosing frequency and cost (reduced drug required per dose).

Clinical evaluation of folate-XTEN-drug composition is conducted in patients with relapsed or refractory advanced tumors or in patients suffering from platinum-resistant ovarian cancer and non-small cell lung carcinoma who have failed using other chemotherapies. Clinical trials are designed to determine the efficacy and advantages of the folate-XTEN-drug conjugate over standard therapies in humans Such studies in patients would comprise three phases. First, a Phase I safety and pharmacokinetics study is conducted to determine the MTD and to characterize the dose-limiting toxicity, pharmacokinetics and preliminary pharmacodynamics in humans. These initial studies could be performed in patients with folate receptor positive status that have relapsed or have refractory advanced tumors and for which standard curative or palliative measures could not be used or were no longer effective or tolerated. The phase I study would use single escalating doses of folate-XTEN-drug conjugate and would measure biochemical, PK, and clinical parameters to permit the determination of the MTD and establish the threshold and maximum concentrations in dosage and in circulating drug that constitute the therapeutic window to be used in subsequent Phase II and Phase III trials as well as defining potential toxicities and adverse events to be tracked in future studies.

Phase II clinical studies of human patients would be independently conducted in folate receptor positive platinum-resistant ovarian cancer patient population, non-small cell lung carcinoma patients having failed numerous chemotherapies, and patients suffering from relapsed or refractory advanced tumors. The trials would evaluate the efficacy and safety of folate-XTEN-drug conjugate alone and in combination with a current chemotherapy employed in the specific indication. Patients will receive intravenously administered folate-XTEN-drug conjugate at a dose level and regimen determined in the Phase I study with or without the standard chemotherapy agent. A control arm comprising of the chemotherapy agent plus placebo would be included. The primary endpoint would be response rate as defined by the Response Evaluation Criteria in Solid Tumors (RECIST). Secondary endpoints will include safety and tolerability, time-to-progression and overall survival.

A phase III efficacy and safety study is conducted in folate-receptor positive platinum-resistant ovarian cancer patients, non-small cell lung carcinoma patients, or advanced tumor relapsed or refractory patients cancer patients to test ability to reach statistically significant clinical endpoints such as progression-free-survival as measured by RECIST. The trial will also be statistically powered for overall survival as a secondary endpoint with projected enrollment in excess of 400 patients. Efficacy outcomes are determined using standard statistical methods. Toxicity and adverse event markers are also followed in the study to verify that the compound is safe when used in the manner described.

### Example 29

### Biodegradation of XTEN in Tissue Homogenate

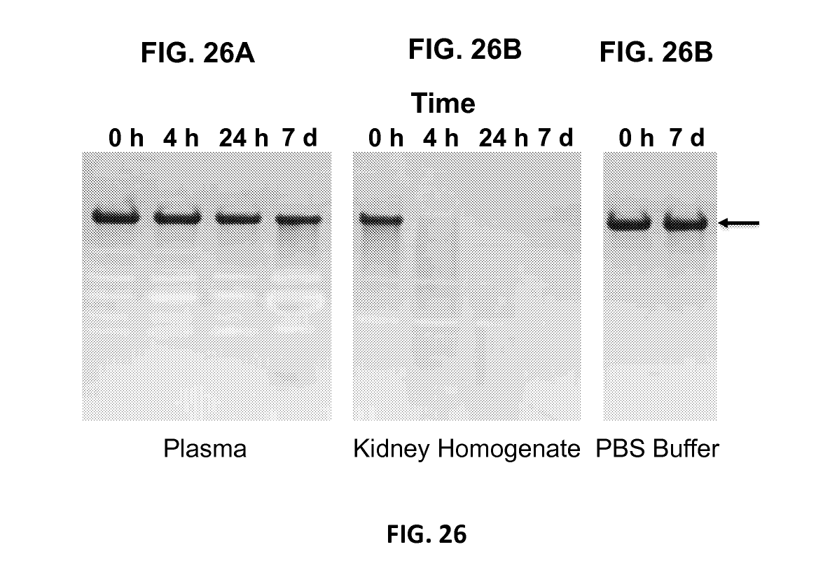

Purified XTEN_AE864 protein was incubated at a final concentration of 0.3 mg/ml in rat plasma (Bioreclamation IVT, Baltimore, Md.), rat kidney homogenate (Bioreclamation), or PBS buffer for up to 7 days at 37° C. Samples were withdrawn at time 0, 4 hours, 24 hours, and 7 days, were immediately frozen and stored at -80° C., then thawed right before analysis. XTEN was extracted from the samples by methanol precipitation. Briefly, methanol pre-chilled at −20° C. was added to the samples at a volume ratio of 2:1 and mixed by vortex. The mixture was kept at −20° C. for 30 min and then centrifuged at 14,000 rpm for 20 min at 8° C. The supernatant was subjected to centrifugal evaporation for 1-2 hours to take the sample to complete dryness, and was then resuspended in PBS buffer to the original volume before methanol extraction. The reconstituted samples were analyzed by SDS-PAGE with staining using Stains-all. The results are shown in FIG. 26. The XTEN_AE864 showed only minor signs of degradation over the 7 day incubation period in both plasma and PBS buffer. However, XTEN_AE864 was rapidly degraded in rat kidney by the 24 hour interval.

### Example 30

### Characterization of Secondary Structure of XTEN

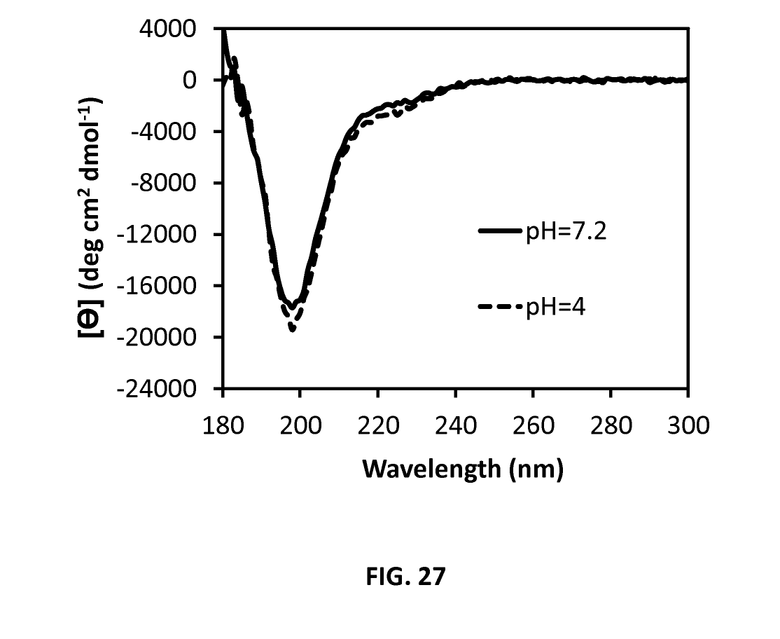

Purified XTEN_AE864 was evaluated for the degree of secondary structure by circular dichroism spectroscopy. CD spectroscopy was performed on a Jasco J850 spectrometer (Jasco, Inc., Easton, Md.) with a Peltier thermal cell holder. The concentration of protein was adjusted to 0.2 mg/mL in 13 mM sodium phosphate buffer at pH 7.2, or 20 mM sodium acetate buffer at pH 4.0. The experiments were carried out using quartz cells with an optical path length of 0.1 cm. The CD spectra were acquired at 20° C. All spectra were recorded in four replicates from 300 nm to 180 nm using a bandwidth of 1 nm. The CD spectra showed similar profiles for XTEN_AE864 at both pH 7.2 and pH 4.0 (FIG. 27) The data are consistent with the spectra of an unstructured polypeptide, where there is no evidence of stable secondary structure.

### Example 31

### Increasing Solubility and Stability of a Peptide Payload by Linking to XTEN

In order to evaluate the ability of XTEN to enhance the physicochemical properties of solubility and stability, fusion proteins of glucagon plus shorter-length XTEN were prepared and evaluated. The test articles were prepared in Tris-buffered saline at neutral pH and characterization of the Gcg-XTEN solution was by reverse-phase HPLC and size exclusion chromatography to affirm that the protein was homogeneous and non-aggregated in solution. The data are presented in Table 35. For comparative purposes, the solubility limit of unmodified glucagon in the same buffer was measured at 60 μM (0.2 mg/mL), and the result demonstrate that for all lengths of XTEN added, a substantial increase in solubility was attained. Importantly, in most cases the glucagon-XTEN fusion proteins were prepared to achieve target concentrations and were not evaluated to determine the maximum solubility limits for the given construct. However, in the case of glucagon linked to the AF-144 XTEN, the limit of solubility was determined, with the result that a 60-fold increase in solubility was achieved, compared to glucagon not linked to XTEN. In addition, the glucagon-AF144 was evaluated for stability, and was found to be stable in liquid formulation for at least 6 months under refrigerated conditions and for approximately one month at 37° C. (data not shown).

The data support the conclusion that the linking of short-length XTEN polypeptides to a biologically active protein such as glucagon can markedly enhance the solubility properties of the protein by the resulting fusion protein, as well as confer stability at the higher protein concentrations.

TABLE 35
Solubility of Glucagon-XTEN constructs

| Test Article | Solubility | |
|---|---|---|
| Glucagon | 60 | μM |
| Glucagon-Y36 | >370 | μM |
| Glucagon-Y72 | >293 | μM |
| Glucagon-AF108 | >145 | μM |
| Glucagon-AF120 | >160 | μM |
| Glucagon-Y144 | >497 | μM |
| Glucagon-AE144 | >467 | μM |
| Glucagon-AF144 | >3600 | μM |
| Glucagon-Y288 | >163 | μM |

### Example 32

### Analysis of Polypeptide Sequences for Repetitiveness

In this Example, different polypeptides, including several XTEN sequences, were assessed for repetitiveness in the amino acid sequence. Polypeptide amino acid sequences can be assessed for repetitiveness by quantifying the number of times a shorter subsequence appears within the overall polypeptide. For example, a polypeptide of 200 amino acid residues length has a total of 165 overlapping 36-amino acid “blocks” (or “36-mers”) and 198 3-mer “subsequences”, but the number of unique 3-mer subsequences will depend on the amount of repetitiveness within the sequence. For the analyses, different polypeptide sequences were assessed for repetitiveness by determining the average subsequence score obtained by application of the following equation:

$$Average subsequence score = \frac{{\sum }_{i = 1}^{n} ( \frac{{Count}_{i}}{m} )}{n}$$

where: n=(amino acid length of polypeptide)−(amino acid length of block)+1;

m=(amino acid length of block)−(amino acid length of subsequence)+1; and

Count,=cumulative number of occurrences of each unique subsequence within block,

In the analyses of the present Example, the average subsequence score for the polypeptides of Table 36 were determined using the foregoing equation in a computer program wherein the block length was set at 36 amino acids and the subsequence length was set at 3 amino acids. The resulting average subsequence score is a reflection of the degree of repetitiveness within the polypeptide.

The results, shown in Table 36, indicate that the polypeptides consisting of 2 or 3 amino acid types have high average subsequence scores and, hence, a high degree of repetitiveness, while XTEN designed with only four types of 12 amino acids motifs (e.g., motifs from a family of Table 9), each consisting of four to six amino acids (i.e., G, S, T, E, P, and A) in a non-repetitive sequence, have average subsequence scores of less than 3 and, in many cases, less than 2, reflecting a low degree of repetitiveness across the entire sequence. For example, the L288 sequence has two amino acid types and has short, highly repetitive block sequences, resulting in a average subsequence score of 8.5. The polypeptide J288 has three amino acid types but also has short, repetitive block sequences, resulting in a average subsequence score of 5.7. Y576 also has three amino acid types, but is not made of internal repeats, reflected in the average subsequence score of 4.7. W576 consists of four types of amino acids, but has a higher degree of internal repetitiveness with the blocks, e.g., “GGSG” (SEQ ID NO: 731), resulting in a average subsequence score of 4.3. The XTEN AD576 consists of four types of 12 amino acid motifs, each consisting of four types of amino acids. Because of the low degree of internal repetitiveness of the individual motifs, the overall average subsequence score amino acids is 2.5. In contrast, the XTEN's consisting of four motifs containing six types of amino acids, each with a low degree of internal repetitiveness, have average subsequence scores less than 2. For the XTEN sequences AE864 and AG864, the output of the program was graphed to show the variation in repetitiveness over the length of the sequence. For AE864 and AG864, the output in which the individual subsequence score for each of the sequential 36-mer blocks were plotted as individual points corresponding to the start of each block as the amino acid number in the sequence in the X axis versus the subsequence scores for the corresponding blocks in the Y-axis showed that for AE864 the sequence, which has an overall average subsequence score of 1.7, varies between scores of 1 and 2 for much of the sequence, but has areas of higher repetitiveness starting around amino acid 330, 505, and 725. Conversely, there are approximately 10 blocks where the subsequence score approaches 1, a score that represents a complete lack of repetitiveness. Similarly, the graph for AG864 showed that the sequence, which has an overall average subsequence score of 1.9, varies between scores of 1.2 and 2 for much of the sequence, but has four areas of higher repetitiveness where the subsequence scores are above 3.

Conclusions: The results indicate that the combination of 12 amino acid subsequence motifs, each consisting of four to six amino acid types that are essentially non-repetitive, into a longer XTEN polypeptide results in an overall sequence that is substantially non-repetitive, as indicated by overall average subsequence scores less than 3 and, in many cases, less than 2. This is despite the fact that each subsequence motif may be used multiple times across the sequence. In contrast, polymers created from smaller numbers of amino acid types resulted in higher average subsequence scores, with polypeptides consisting of two amino acid type having higher scores that those consisting of three amino acid types.

TABLE 36
Average subsequence score calculations of nolvueutide sequences

| | | | SEQ ID |
|---|---|---|---|
| Seq Name | Amino Acid Sequence | Score | NO: |
| H288 | GSGGEGGSGGSGGSGGEGGSGGSGGSGGEGGSGGSGGSGGEG | 7.1 | 732 |
| | GSGGSGGSGGEGGSGGSGGSGGEGGSGGSGGSGGEGGSGGSG | | |
| | GSGGEGGSGGSGGSGGEGGSGGSGGSGGEGGSGGSGGSGGEG | | |
| | GSGGSGGSGGEGGSGGSGGSGGEGGSGGSGGSGGEGGSGGSG | | |
| | GSGGEGGSGGSGGSGGEGGSGGSGGSGGEGGSGGSGGSGGEG | | |
| | GSGGSGGSGGEGGSGGSGGSGGEGGSGGSGGSGGEGGSGGSG | | |
| | GSGGEGGSGGSGGSGGEGGSGGSGGSGGEGGSGGSG | | |
| J288 | GSGGEGGSGGEGGSGGEGGSGGEGGSGGEGGSGGEGGSGGEG | 5.7 | 733 |
| | GSGGEGGSGGEGGSGGEGGSGGEGGSGGEGGSGGEGGSGGEG | | |
| | GSGGEGGSGGEGGSGGEGGSGGEGGSGGEGGSGGEGGSGGEG | | |
| | GSGGEGGSGGEGGSGGEGGSGGEGGSGGEGGSGGEGGSGGEG | | |
| | GSGGEGGSGGEGGSGGEGGSGGEGGSGGEGGSGGEGGSGGEG | | |
| | GSGGEGGSGGEGGSGGEGGSGGEGGSGGEGGSGGEGGSGGEG | | |
| | GSGGEGGSGGEGGSGGEGGSGGEGGSGGEGGSGGEG | | |
| K288 | GEGEGGGEGGEGEGGGEGGEGEGGGEGGEGEGGGEGGEGEGG | 8.0 | 734 |
| | GEGGEGEGGGEGGEGEGGGEGGEGEGGGEGGEGEGGGEGGEG | | |
| | EGGGEGGEGEGGGEGGEGEGGGEGGEGEGGGEGGEGEGGGEG | | |
| | GEGEGGGEGGEGEGGGEGGEGEGGGEGGEGEGGGEGGEGEGG | | |
| | GEGGEGEGGGEGGEGEGGGEGGEGEGGGEGGEGEGGGEGGEG | | |
| | EGGGEGGEGEGGGEGGEGEGGGEGGEGEGGGEGGEGEGGGEG | | |
| | GEGEGGGEGGEGEGGGEGGEGEGGGEGGEGEGGGEG | | |
| L288 | SSESSESSSSESSSESSESSSSESSSESSESSSSESSSESSESSSSESSS | 8.5 | 735 |
| | ESSESSSSESSSESSESSSSESSSESSESSSSESSSESSESSSSESSSES | | |
| | SESSSSESSSESSESSSSESSSESSESSSSESSSESSESSSSESSSESSE | | |
| | SSSSESSSESSESSSSESSSESSESSSSESSSESSESSSSESSSESSESS | | |
| | SSESSSESSESSSSESSSESSESSSSESSSESSESSSSESSSESSESSSS | | |
| | ESSSESSESSSSESSSESSESSSSESSSESSESSSSES | | |
| Y288 | GEGSGEGSEGEGSEGSGEGEGSEGSGEGEGGSEGSEGEGGSEGS | 4.7 | 736 |
| | EGEGGSEGSEGEGSGEGSEGEGGSEGSEGEGSGEGSEGEGSEGG | | |
| | SEGEGGSEGSEGEGSGEGSEGEGGEGGSEGEGSEGSGEGEGSGE | | |
| | GSEGEGSEGSGEGEGSGEGSEGEGSEGSGEGEGSEGSGEGEGGS | | |
| | EGSEGEGSEGSGEGEGGEGSGEGEGSGEGSEGEGGGEGSEGEGS | | |
| | GEGGEGEGSEGGSEGEGGSEGGEGEGSEGSGEGEGSEGGSEGE | | |
| | GSEGGSEGEGSEGSGEGEGSEGSGE | | |
| AE42_1 | TEPSEGSAPGSPAGSPTSTEEGTSESATPESGPGSEPATSGS | 1.2 | 737 |
| AE42_2 | PAGSPTSTEEGTSTEPSEGSAPGTSESATPESGPGSEPATSG | 1.2 | 738 |
| AE42_3 | SEPATSGSETPGTSESATPESGPGTSTEPSEGSAPGSPAGSP | 1.1 | 739 |
| AG42_1 | GAPSPSASTGTGPGTPGSGTASSSPGSSTPSGATGSPGPSGP | 1.1 | 740 |
| AG42_2 | GPGTPGSGTASSSPGSSTPSGATGSPGSSPSASTGTGPGASP | 1.3 | 741 |
| AG42_3 | SPSASTGTGPGASPGTSSTGSPGTPGSGTASSSPGSSTPSGA | 1.4 | 742 |
| AG42_4 | SASTGTGPGASPGTSSTGSPGTPGSGTASSSPGSSTPSGATG | 1.4 | 743 |
| AE48 | MAEPAGSPTSTEEGTPGSGTASSSPGSSTPSGATGSPGASPGTSST | 1.2 | 744 |
| | GS | | |
| AM48 | MAEPAGSPTSTEEGASPGTSSTGSPGSSTPSGATGSPGSSTPSGAT | 1.7 | 745 |
| | GS | | |
| AE144 | GSEPATSGSETPGTSESATPESGPGSEPATSGSETPGSPAGSPTST | 1.6 | 746 |
| | EEGTSTEPSEGSAPGSEPATSGSETPGSEPATSGSETPGSEPATSG | | |
| | SETPGTSTEPSEGSAPGTSESATPESGPGSEPATSGSETPGTSTEPS | | |
| | EGSAP | | |
| AF144 | GTSTPESGSASPGTSPSGESSTAPGTSPSGESSTAPGSTSSTAESPG | 1.7 | 747 |
| | PGSTSESPSGTAPGSTSSTAESPGPGTSPSGESSTAPGTSTPESGSA | | |
| | SPGSTSSTAESPGPGTSPSGESSTAPGTSPSGESSTAPGTSPSGESS | | |
| | TAP | | |
| AG144_1 | PGSSPSASTGTGPGSSPSASTGTGPGTPGSGTASSSPGSSTPSGAT | 1.6 | 748 |
| | GSPGSSPSASTGTGPGASPGTSSTGSPGTPGSGTASSSPGSSTPSG | | |
| | ATGSPGTPGSGTASSSPGASPGTSSTGSPGASPGTSSTGSPGTPGS | | |
| | GTASSS | | |
| AG144_2 | SGTASSSPGSSTPSGATGSPGTPGSGTASSSPGSSTPSGATGSPGS | 1.7 | 749 |
| | STPSGATGSPGSSPSASTGTGPGSSPSASTGTGPGASPGTSSTGSP | | |
| | GTPGSGTASSSPGSSTPSGATGSPGSSPSASTGTGPGSSPSASTGT | | |
| | GPGASP | | |
| AG144_3 | GTPGSGTASSSPGSSTPSGATGSPGSSTPSGATGSPGSSPSASTGT | 1.7 | 750 |
| | GPGSSPSASTGTGPGASPGTSSTGSPGTPGSGTASSSPGSSTPSGA | | |
| | TGSPGSSPSASTGTGPGSSPSASTGTGPGASPGTSSTGSPGASPGT | | |
| | SSTGSP | | |
| AG144_4 | GTPGSGTASSSPGSSTPSGATGSPGSSPSASTGTGPGSSPSASTGT | 1.7 | 751 |
| | GPGASPGTSSTGSPGASPGTSSTGSPGSSTPSGATGSPGSSPSAST | | |
| | GTGPGASPGTSSTGSPGSSPSASTGTGPGTPGSGTASSSPGSSTPS | | |
| | GATGSP | | |
| AE288 | GTSESATPESGPGSEPATSGSETPGTSESATPESGPGSEPATSGSE | 1.6 | 752 |
| | TPGTSESATPESGPGTSTEPSEGSAPGSPAGSPTSTEEGTSESATP | | |
| | ESGPGSEPATSGSETPGTSESATPESGPGSPAGSPTSTEEGSPAGS | | |
| | PTSTEEGTSTEPSEGSAPGTSESATPESGPGTSESATPESGPGTSES | | |
| | ATPESGPGSEPATSGSETPGSEPATSGSETPGSPAGSPTSTEEGTS | | |
| | TEPSEGSAPGTSTEPSEGSAPGSEPATSGSETPGTSESATPESGPG | | |
| | TSTEPSEGSAP | | |
| AG288_1 | PGASPGTSSTGSPGASPGTSSTGSPGTPGSGTASSSPGSSTPSGAT | 1.8 | 753 |
| | GSPGTPGSGTASSSPGSSTPSGATGSPGTPGSGTASSSPGSSTPSG | | |
| | ATGSPGSSTPSGATGSPGSSPSASTGTGPGSSPSASTGTGPGASPG | | |
| | TSSTGSPGTPGSGTASSSPGSSTPSGATGSPGSSPSASTGTGPGSS | | |
| | PSASTGTGPGASPGTSSTGSPGASPGTSSTGSPGSSTPSGATGSPG | | |
| | SSPSASTGTGPGASPGTSSTGSPGSSPSASTGTGPGTPGSGTASSS | | |
| | PGSSTPSGATGS | | |
| AG288_2 | GSSPSASTGTGPGSSPSASTGTGPGTPGSGTASSSPGSSTPSGATG | 1.8 | 754 |
| | SPGSSPSASTGTGPGASPGTSSTGSPGTPGSGTASSSPGSSTPSGA | | |
| | TGSPGTPGSGTASSSPGASPGTSSTGSPGASPGTSSTGSPGTPGSG | | |
| | TASSSPGSSTPSGATGSPGASPGTSSTGSPGTPGSGTASSSPGSST | | |
| | PSGATGSPGSSPSASTGTGPGSSPSASTGTGPGSSTPSGATGSPGS | | |
| | STPSGATGSPGASPGTSSTGSPGASPGTSSTGSPGASPGTSSTGSP | | |
| | GTPGSGTASSSP | | |
| AD576 | GSSESGSSEGGPGSGGEPSESGSSGSSESGSSEGGPGSSESGSSEG | 2.5 | 755 |
| | GPGSSESGSSEGGPGSSESGSSEGGPGSSESGSSEGGPGESPGGSS | | |
| | GSESGSEGSSGPGESSGSSESGSSEGGPGSSESGSSEGGPGSSESG | | |
| | SSEGGPGSGGEPSESGSSGESPGGSSGSESGESPGGSSGSESGSGG | | |
| | EPSESGSSGSSESGSSEGGPGSGGEPSESGSSGSGGEPSESGSSGS | | |
| | EGSSGPGESSGESPGGSSGSESGSGGEPSESGSSGSGGEPSESGSS | | |
| | GSGGEPSESGSSGSSESGSSEGGPGESPGGSSGSESGESPGGSSGS | | |
| | ESGESPGGSSGSESGESPGGSSGSESGESPGGSSGSESGSSESGSS | | |
| | EGGPGSGGEPSESGSSGSEGSSGPGESSGSSESGSSEGGPGSGGEP | | |
| | SESGSSGSSESGSSEGGPGSGGEPSESGSSGESPGGSSGSESGESP | | |
| | GGSSGSESGSSESGSSEGGPGSGGEPSESGSSGSSESGSSEGGPGS | | |
| | GGEPSESGSSGSGGEPSESGSSGESPGGSSGSESGSEGSSGPGESS | | |
| | GSSESGSSEGGPGSEGSSGPGESS | | |
| AE576 | AGSPAGSPTSTEEGTSESATPESGPGTSTEPSEGSAPGSPAGSPTS | 1.7 | 756 |
| | TEEGTSTEPSEGSAPGTSTEPSEGSAPGTSESATPESGPGSEPATS | | |
| | GSETPGSEPATSGSETPGSPAGSPTSTEEGTSESATPESGPGTSTE | | |
| | PSEGSAPGTSTEPSEGSAPGSPAGSPTSTEEGTSTEPSEGSAPGTS | | |
| | TEPSEGSAPGTSESATPESGPGTSTEPSEGSAPGTSESATPESGPG | | |
| | SEPATSGSETPGTSTEPSEGSAPGTSTEPSEGSAPGTSESATPESGP | | |
| | GTSESATPESGPGSPAGSPTSTEEGTSESATPESGPGSEPATSGSE | | |
| | TPGTSESATPESGPGTSTEPSEGSAPGTSTEPSEGSAPGTSTEPSE | | |
| | GSAPGTSTEPSEGSAPGTSTEPSEGSAPGTSTEPSEGSAPGSPAGS | | |
| | PTSTEEGTSTEPSEGSAPGTSESATPESGPGSEPATSGSETPGTSES | | |
| | ATPESGPGSEPATSGSETPGTSESATPESGPGTSTEPSEGSAPGTS | | |
| | ESATPESGPGSPAGSPTSTEEGSPAGSPTSTEEGSPAGSPTSTEEG | | |
| | TSESATPESGPGTSTEPSEGSAP | | |
| AF540 | GSTSSTAESPGPGSTSSTAESPGPGSTSESPSGTAPGSTSSTAESPG | 1.8 | 757 |
| | PGSTSSTAESPGPGTSTPESGSASPGSTSESPSGTAPGTSPSGESST | | |
| | APGSTSESPSGTAPGSTSESPSGTAPGTSPSGESSTAPGSTSESPSG | | |
| | TAPGSTSESPSGTAPGTSPSGESSTAPGSTSESPSGTAPGSTSESPS | | |
| | GTAPGSTSESPSGTAPGTSTPESGSASPGSTSESPSGTAPGTSTPES | | |
| | GSASPGSTSSTAESPGPGSTSSTAESPGPGTSTPESGSASPGTSTPE | | |
| | SGSASPGSTSESPSGTAPGTSTPESGSASPGTSTPESGSASPGSTSE | | |
| | SPSGTAPGSTSESPSGTAPGSTSESPSGTAPGSTSSTAESPGPGTST | | |
| | PESGSASPGTSTPESGSASPGSTSESPSGTAPGSTSESPSGTAPGTS | | |
| | TPESGSASPGSTSESPSGTAPGSTSESPSGTAPGTSTPESGSASPGT | | |
| | SPSGESSTAPGSTSSTAESPGPGTSPSGESSTAPGSTSSTAESPGPG | | |
| | TSTPESGSASPGSTSESPSGTAP | | |
| AF504 | GASPGTSSTGSPGSSPSASTGTGPGSSPSASTGTGPGTPGSGTASS | 1.9 | 758 |
| | SPGSSTPSGATGSPGSNPSASTGTGPGASPGTSSTGSPGTPGSGTA | | |
| | SSSPGSSTPSGATGSPGTPGSGTASSSPGASPGTSSTGSPGASPGT | | |
| | SSTGSPGTPGSGTASSSPGSSTPSGATGSPGASPGTSSTGSPGTPG | | |
| | SGTASSSPGSSTPSGATGSPGSNPSASTGTGPGSSPSASTGTGPGS | | |
| | STPSGATGSPGSSTPSGATGSPGASPGTSSTGSPGASPGTSSTGSP | | |
| | GASPGTSSTGSPGTPGSGTASSSPGASPGTSSTGSPGASPGTSSTG | | |
| | SPGASPGTSSTGSPGSSPSASTGTGPGTPGSGTASSSPGASPGTSS | | |
| | TGSPGASPGTSSTGSPGASPGTSSTGSPGSSTPSGATGSPGSSTPS | | |
| | GATGSPGASPGTSSTGSPGTPGSGTASSSPGSSTPSGATGSPGSST | | |
| | PSGATGSPGSSTPSGATGSPGSSPSASTGTGPGASPGTSSTGSP | | |
| AGS576 | PGTPGSGTASSSPGSSTPSGATGSPGSSPSASTGTGPGSSPSASTG | 2.1 | 759 |
| | TGPGSSTPSGATGSPGSSTPSGATGSPGASPGTSSTGSPGASPGTS | | |
| | STGSPGASPGTSSTGSPGTPGSGTASSSPGASPGTSSTGSPGASPG | | |
| | TSSTGSPGASPGTSSTGSPGSSPSASTGTGPGTPGSGTASSSPGAS | | |
| | PGTSSTGSPGASPGTSSTGSPGASPGTSSTGSPGSSTPSGATGSPG | | |
| | SSTPSGATGSPGASPGTSSTGSPGTPGSGTASSSPGSSTPSGATGS | | |
| | PGSSTPSGATGSPGSSTPSGATGSPGSSPSASTGTGPGASPGTSST | | |
| | GSPGASPGTSSTGSPGTPGSGTASSSPGASPGTSSTGSPGASPGTS | | |
| | STGSPGASPGTSSTGSPGASPGTSSTGSPGTPGSGTASSSPGSSTP | | |
| | SGATGSPGTPGSGTASSSPGSSTPSGATGSPGTPGSGTASSSPGSS | | |
| | TPSGATGSPGSSTPSGATGSPGSSPSASTGTGPGSSPSASTGTGPG | | |
| | ASPGTSSTGSPGTPGSGTASSSPGSSTPSGATGSPGSSPSASTGTG | | |
| | PGSSPSASTGTGPGASPGTSSTGS | | |
| AD836 | GSSESGSSEGGPGSSESGSSEGGPGESPGGSSGSESGSGGEPSESG | 2.5 | 760 |
| | SSGESPGGSSGSESGESPGGSSGSESGSSESGSSEGGPGSSESGSS | | |
| | EGGPGSSESGSSEGGPGESPGGSSGSESGESPGGSSGSESGESPGG | | |
| | SSGSESGSSESGSSEGGPGSSESGSSEGGPGSSESGSSEGGPGSSE | | |
| | SGSSEGGPGSSESGSSEGGPGSSESGSSEGGPGSGGEPSESGSSGE | | |
| | SPGGSSGSESGESPGGSSGSESGSGGEPSESGSSGSEGSSGPGESS | | |
| | GSSESGSSEGGPGSGGEPSESGSSGSEGSSGPGESSGSSESGSSEG | | |
| | GPGSGGEPSESGSSGESPGGSSGSESGSGGEPSESGSSGSGGEPSE | | |
| | SGSSGSSESGSSEGGPGSGGEPSESGSSGSGGEPSESGSSGSEGSS | | |
| | GPGESSGESPGGSSGSESGSEGSSGPGESSGSEGSSGPGESSGSGG | | |
| | EPSESGSSGSSESGSSEGGPGSSESGSSEGGPGESPGGSSGSESGS | | |
| | GGEPSESGSSGSEGSSGPGESSGESPGGSSGSESGSEGSSGPGSSE | | |
| | SGSSEGGPGSGGEPSESGSSGSEGSSGPGESSGSEGSSGPGESSGS | | |
| | EGSSGPGESSGSGGEPSESGSSGSGGEPSESGSSGESPGGSSGSES | | |
| | GESPGGSSGSESGSGGEPSESGSSGSEGSSGPGESSGESPGGSSGS | | |
| | ESGSSESGSSEGGPGSSESGSSEGGPGSSESGSSEGGPGSGGEPSE | | |
| | SGSSGSSESGSSEGGPGESPGGSSGSESGSGGEPSESGSSGSSESG | | |
| | SSEGGPGESPGGSSGSESGSGGEPSESGSSGESPGGSSGSESGSGG | | |
| | EPSESGSS | | |
| AE864 | GSPAGSPTSTEEGTSESATPESGPGTSTEPSEGSAPGSPAGSPTST | 1.7 | 761 |
| | EEGTSTEPSEGSAPGTSTEPSEGSAPGTSESATPESGPGSEPATSG | | |
| | SETPGSEPATSGSETPGSPAGSPTSTEEGTSESATPESGPGTSTEPS | | |
| | GSAPGTSTEPSEGSAPGSPAGSPTSTEEGTSTEPSEGSAPGTSTE | | |
| | PSEGSAPGTSESATPESGPGTSTEPSEGSAPGTSESATPESGPGSE | | |
| | PATSGSETPGTSTEPSEGSAPGTSTEPSEGSAPGTSESATPESGPG | | |
| | TSESATPESGPGSPAGSPTSTEEGTSESATPESGPGSEPATSGSETP | | |
| | GTSESATPESGPGTSTEPSEGSAPGTSTEPSEGSAPGTSTEPSEGS | | |
| | APGTSTEPSEGSAPGTSTEPSEGSAPGTSTEPSEGSAPGSPAGSPT | | |
| | STEEGTSTEPSEGSAPGTSESATPESGPGSEPATSGSETPGTSESA | | |
| | TPESGPGSEPATSGSETPGTSESATPESGPGTSTEPSEGSAPGTSES | | |
| | ATPESGPGSPAGSPTSTEEGSPAGSPTSTEEGSPAGSPTSTEEGTS | | |
| | ESATPESGPGTSTEPSEGSAPGTSESATPESGPGSEPATSGSETPG | | |
| | TSESATPESGPGSEPATSGSETPGTSESATPESGPGTSTEPSEGSAP | | |
| | GSPAGSPTSTEEGTSESATPESGPGSEPATSGSETPGTSESATPES | | |
| | GPGSPAGSPTSTEEGSPAGSPTSTEEGTSTEPSEGSAPGTSESATP | | |
| | ESGPGTSESATPESGPGTSESATPESGPGSEPATSGSETPGSEPAT | | |
| | SGSETPGSPAGSPTSTEEGTSTEPSEGSAP | | |
| AF864 | GSTSSTAESPGPGSTSSTAESPGPGSTSESPSGTAPGSTSSTAESPG | 1.8 | 762 |
| | PGSTSSTAESPGPGTSTPESGSASPGSTSESPSGTAPGTSPSGESST | | |
| | APGSTSESPSGTAPGSTSESPSGTAPGTSPSGESSTAPGSTSESPSG | | |
| | TAPGSTSESPSGTAPGTSPSGESSTAPGSTSESPSGTAPGSTSESPS | | |
| | GTAPGSTSESPSGTAPGTSTPESGSASPGSTSESPSGTAPGTSTPES | | |
| | GSASPGSTSSTAESPGPGSTSSTAESPGPGTSTPESGSASPGTSTPE | | |
| | SGSASPGSTSESPSGTAPGTSTPESGSASPGTSTPESGSASPGSTSE | | |
| | SPSGTAPGSTSESPSGTAPGSTSESPSGTAPGSTSSTAESPGPGTST | | |
| | PESGSASPGTSTPESGSASPGSTSESPSGTAPGSTSESPSGTAPGTS | | |
| | TPESGSASPGSTSESPSGTAPGSTSESPSGTAPGTSTPESGSASPGT | | |
| | SPSGESSTAPGSTSSTAESPGPGTSPSGESSTAPGSTSSTAESPGPG | | |
| | TSTPESGSASPGSTSESPSGTAPGSTSSTAESPGPGTSTPESGSASP | | |
| | GTSTPESGSASPGSTSSTAESPGPGTSPSGESSTAPGTSTPESGSAS | | |
| | PGSTSESPSGTAPGSTSESPSGTAPGTSTPESGSASPGTSPSGESST | | |
| | APGSTSSTAESPGPGTSPSGESSTAPGSTSSTAESPGPGSTSSTAES | | |
| | PGPGTSPSGESSTAPGSTSESPSGTAPGSTSESPSGTAPGTSTPESG | | |
| | SASPGSTSESPSGTAPGSTSESPSGTAPGTSTPESGSASPGSTSESP | | |
| | SGTAPGSTSESPSGTAPGSTSESPSGTAPGSTSSTAESPGPGTSPS | | |
| | GESSTAPGTSPSGESSTAP | | |
| AG864 | GASPGTSSTGSPGSSPSASTGTGPGSSPSASTGTGPGTPGSGTASS | 1.9 | 763 |
| | SPGSSTPSGATGSPGSSPSASTGTGPGASPGTSSTGSPGTPGSGTA | | |
| | SSSPGSSTPSGATGSPGTPGSGTASSSPGASPGTSSTGSPGASPGT | | |
| | SSTGSPGTPGSGTASSSPGSSTPSGATGSPGASPGTSSTGSPGTPG | | |
| | SGTASSSPGSSTPSGATGSPGSSPSASTGTGPGSSPSASTGTGPGS | | |
| | STPSGATGSPGSSTPSGATGSPGASPGTSSTGSPGASPGTSSTGSP | | |
| | GASPGTSSTGSPGTPGSGTASSSPGASPGTSSTGSPGASPGTSSTG | | |
| | SPGASPGTSSTGSPGSSPSASTGTGPGTPGSGTASSSPGASPGTSS | | |
| | TGSPGASPGTSSTGSPGASPGTSSTGSPGSSTPSGATGSPGSSTPS | | |
| | GATGSPGASPGTSSTGSPGTPGSGTASSSPGSSTPSGATGSPGSST | | |
| | PSGATGSPGSSTPSGATGSPGSSPSASTGTGPGASPGTSSTGSPGA | | |
| | SPGTSSTGSPGTPGSGTASSSPGASPGTSSTGSPGASPGTSSTGSP | | |
| | GASPGTSSTGSPGASPGTSSTGSPGTPGSGTASSSPGSSTPSGATG | | |
| | SPGTPGSGTASSSPGSSTPSGATGSPGTPGSGTASSSPGSSTPSGA | | |
| | TGSPGSSTPSGATGSPGSSPSASTGTGPGSSPSASTGTGPGASPGT | | |
| | SSTGSPGTPGSGTASSSPGSSTPSGATGSPGSSPSASTGTGPGSSPS | | |
| | ASTGTGPGASPGTSSTGSPGASPGTSSTGSPGSSTPSGATGSPGSS | | |
| | PSASTGTGPGASPGTSSTGSPGSSPSASTGTGPGTPGSGTASSSPG | | |
| | SSTPSGATGSPGSSTPSGATGSPGASPGTSSTGSP | | |
| AM875 | GTSTEPSEGSAPGSEPATSGSETPGSPAGSPTSTEEGSTSSTAESP | 1.5 | 764 |
| | GPGTSTPESGSASPGSTSESPSGTAPGSTSESPSGTAPGTSTPESGS | | |
| | ASPGTSTPESGSASPGSEPATSGSETPGTSESATPESGPGSPAGSP | | |
| | TSTEEGTSTEPSEGSAPGTSESATPESGPGTSTEPSEGSAPGTSTEP | | |
| | SEGSAPGSPAGSPTSTEEGTSTEPSEGSAPGTSTEPSEGSAPGTSE | | |
| | SATPESGPGTSESATPESGPGTSTEPSEGSAPGTSTEPSEGSAPGT | | |
| | SESATPESGPGTSTEPSEGSAPGSEPATSGSETPGSPAGSPTSTEE | | |
| | GSSTPSGATGSPGTPGSGTASSSPGSSTPSGATGSPGTSTEPSEGS | | |
| | APGTSTEPSEGSAPGSEPATSGSETPGSPAGSPTSTEEGSPAGSPT | | |
| | STEEGTSTEPSEGSAPGASASGAPSTGGTSESATPESGPGSPAGSP | | |
| | TSTEEGSPAGSPTSTEEGSTSSTAESPGPGSTSESPSGTAPGTSPSG | | |
| | ESSTAPGTPGSGTASSSPGSSTPSGATGSPGSSPSASTGTGPGSEP | | |
| | ATSGSETPGTSESATPESGPGSEPATSGSETPGSTSSTAESPGPGS | | |
| | TSSTAESPGPGTSPSGESSTAPGSEPATSGSETPGSEPATSGSETP | | |
| | GTSTEPSEGSAPGSTSSTAESPGPGTSTPESGSASPGSTSESPSGTA | | |
| | PGTSTEPSEGSAPGTSTEPSEGSAPGTSTEPSEGSAPGSSTPSGAT | | |
| | GSPGSSPSASTGTGPGASPGTSSTGSPGSEPATSGSETPGTSESAT | | |
| | PESGPGSPAGSPTSTEEGSSTPSGATGSPGSSPSASTGTGPGASPG | | |
| | TSSTGSPGTSESATPESGPGTSTEPSEGSAPGTSTEPSEGSAP | | |
| AM1296 | GTSTEPSEGSAPGSEPATSGSETPGSPAGSPTSTEEGSTSSTAESP | 1.6 | 765 |
| | GPGTSTPESGSASPGSTSESPSGTAPGSTSESPSGTAPGTSTPESGS | | |
| | ASPGTSTPESGSASPGSEPATSGSETPGTSESATPESGPGSPAGSP | | |
| | TSTEEGTSTEPSEGSAPGTSESATPESGPGTSTEPSEGSAPGTSTEP | | |
| | SEGSAPGSPAGSPTSTEEGTSTEPSEGSAPGTSTEPSEGSAPGTSE | | |
| | SATPESGPGTSESATPESGPGTSTEPSEGSAPGTSTEPSEGSAPGT | | |
| | SESATPESGPGTSTEPSEGSAPGSEPATSGSETPGSPAGSPTSTEE | | |
| | GSSTPSGATGSPGTPGSGTASSSPGSSTPSGATGSPGTSTEPSEGS | | |
| | APGTSTEPSEGSAPGSEPATSGSETPGSPAGSPTSTEEGSPAGSPT | | |
| | STEEGTSTEPSEGSAPGPEPTGPAPSGGSEPATSGSETPGTSESAT | | |
| | PESGPGSPAGSPTSTEEGTSESATPESGPGSPAGSPTSTEEGSPAG | | |
| | SPTSTEEGTSESATPESGPGSPAGSPTSTEEGSPAGSPTSTEEGSTS | | |
| | STAESPGPGSTSESPSGTAPGTSPSGESSTAPGSTSESPSGTAPGST | | |
| | SESPSGTAPGTSPSGESSTAPGTSTEPSEGSAPGTSESATPESGPG | | |
| | TSESATPESGPGSEPATSGSETPGTSESATPESGPGTSESATPESGP | | |
| | GTSTEPSEGSAPGTSESATPESGPGTSTEPSEGSAPGTSPSGESST | | |
| | APGTSPSGESSTAPGTSPSGESSTAPGTSTEPSEGSAPGSPAGSPT | | |
| | STEEGTSTEPSEGSAPGSSPSASTGTGPGSSTPSGATGSPGSSTPS | | |
| | GATGSPGSSTPSGATGSPGSSTPSGATGSPGASPGTSSTGSPGAS | | |
| | ASGAPSTGGTSPSGESSTAPGSTSSTAESPGPGTSPSGESSTAPGT | | |
| | SESATPESGPGTSTEPSEGSAPGTSTEPSEGSAPGSSPSASTGTGP | | |
| | GSSTPSGATGSPGASPGTSSTGSPGTSTPESGSASPGTSPSGESST | | |
| | APGTSPSGESSTAPGTSESATPESGPGSEPATSGSETPGTSTEPSE | | |
| | GSAPGSTSESPSGTAPGSTSESPSGTAPGTSTPESGSASPGSPAGS | | |
| | PTSTEEGTSESATPESGPGTSTEPSEGSAPGSPAGSPTSTEEGTSES | | |
| | ATPESGPGSEPATSGSETPGSSTPSGATGSPGASPGTSSTGSPGSS | | |
| | TPSGATGSPGSTSESPSGTAPGTSPSGESSTAPGSTSSTAESPGPG | | |
| | SSTPSGATGSPGASPGTSSTGSPGTPGSGTASSSPGSPAGSPTSTE | | |
| | EGSPAGSPTSTEEGTSTEPSEGSAP | | |
| AM923 | MAEPAGSPTSTEEGASPGTSSTGSPGSSTPSGATGSPGSSTPSGAT | 1.5 | 766 |
| | GSPGTSTEPSEGSAPGSEPATSGSETPGSPAGSPTSTEEGSTSSTA | | |
| | ESPGPGTSTPESGSASPGSTSESPSGTAPGSTSESPSGTAPGTSTPE | | |
| | SGSASPGTSTPESGSASPGSEPATSGSETPGTSESATPESGPGSPA | | |
| | GSPTSTEEGTSTEPSEGSAPGTSESATPESGPGTSTEPSEGSAPGT | | |
| | STEPSEGSAPGSPAGSPTSTEEGTSTEPSEGSAPGTSTEPSEGSAP | | |
| | GTSESATPESGPGTSESATPESGPGTSTEPSEGSAPGTSTEPSEGS | | |
| | APGTSESATPESGPGTSTEPSEGSAPGSEPATSGSETPGSPAGSPT | | |
| | STEEGSSTPSGATGSPGTPGSGTASSSPGSSTPSGATGSPGTSTEP | | |
| | SEGSAPGTSTEPSEGSAPGSEPATSGSETPGSPAGSPTSTEEGSPA | | |
| | GSPTSTEEGTSTEPSEGSAPGASASGAPSTGGTSESATPESGPGSP | | |
| | AGSPTSTEEGSPAGSPTSTEEGSTSSTAESPGPGSTSESPSGTAPG | | |
| | TSPSGESSTAPGTPGSGTASSSPGSSTPSGATGSPGSSPSASTGTG | | |
| | PGSEPATSGSETPGTSESATPESGPGSEPATSGSETPGSTSSTAESP | | |
| | GPGSTSSTAESPGPGTSPSGESSTAPGSEPATSGSETPGSEPATSG | | |
| | SETPGTSTEPSEGSAPGSTSSTAESPGPGTSTPESGSASPGSTSESP | | |
| | SGTAPGTSTEPSEGSAPGTSTEPSEGSAPGTSTEPSEGSAPGSSTP | | |
| | SGATGSPGSSPSASTGTGPGASPGTSSTGSPGSEPATSGSETPGTS | | |
| | ESATPESGPGSPAGSPTSTEEGSSTPSGATGSPGSSPSASTGTGPG | | |
| | ASPGTSSTGSPGTSESATPESGPGTSTEPSEGSAPGTSTEPSEGSA | | |
| | P | | |
| AE912 | MAEPAGSPTSTEEGTPGSGTASSSPGSSTPSGATGSPGASPGTSST | 1.7 | 767 |
| | GSPGSPAGSPTSTEEGTSESATPESGPGTSTEPSEGSAPGSPAGSP | | |
| | TSTEEGTSTEPSEGSAPGTSTEPSEGSAPGTSESATPESGPGSEPA | | |
| | TSGSETPGSEPATSGSETPGSPAGSPTSTEEGTSESATPESGPGTS | | |
| | TEPSEGSAPGTSTEPSEGSAPGSPAGSPTSTEEGTSTEPSEGSAPG | | |
| | TSTEPSEGSAPGTSESATPESGPGTSTEPSEGSAPGTSESATPESGP | | |
| | GSEPATSGSETPGTSTEPSEGSAPGTSTEPSEGSAPGTSESATPES | | |
| | GPGTSESATPESGPGSPAGSPTSTEEGTSESATPESGPGSEPATSG | | |
| | SETPGTSESATPESGPGTSTEPSEGSAPGTSTEPSEGSAPGTSTEPS | | |
| | EGSAPGTSTEPSEGSAPGTSTEPSEGSAPGTSTEPSEGSAPGSPAG | | |
| | SPTSTEEGTSTEPSEGSAPGTSESATPESGPGSEPATSGSETPGTSE | | |
| | SATPESGPGSEPATSGSETPGTSESATPESGPGTSTEPSEGSAPGT | | |
| | SESATPESGPGSPAGSPTSTEEGSPAGSPTSTEEGSPAGSPTSTEE | | |
| | GTSESATPESGPGTSTEPSEGSAPGTSESATPESGPGSEPATSGSE | | |
| | TPGTSESATPESGPGSEPATSGSETPGTSESATPESGPGTSTEPSE | | |
| | GSAPGSPAGSPTSTEEGTSESATPESGPGSEPATSGSETPGTSESA | | |
| | TPESGPGSPAGSPTSTEEGSPAGSPTSTEEGTSTEPSEGSAPGTSES | | |
| | ATPESGPGTSESATPESGPGTSESATPESGPGSEPATSGSETPGSE | | |
| | PATSGSETPGSPAGSPTSTEEGTSTEPSEGSAPGTSTEPSEGSAPG | | |
| | SEPATSGSETPGTSESATPESGPGTSTEPSEGSAP | | |

### Example 33

### Selective Cytotoxicity of 3xFA(γ),3xMMAE-XTEN on KB Cells

The ability to selectively target and kill cells bearing folate receptors was evaluated. Test articles of free MMAE, a non-targeting 3xMMAE-XTEN conjugate (XTEN linked to toxin) and the folate receptor-targeted 3xFA(γ),3xMMAE-XTEN conjugate were evaluated in a CellTiter-Glo anti-proliferation assay using the folate receptor-positive KB cell line. As culture media contain high folic acid content, KB cells were grown in folic acid-free media containing 10% heat-inactivated fetal calf serum at 37° C., 5% CO2 for at least 7 days prior to the commencement of the cell viability experiment, This medium was also utilized for the execution of the experiment. In brief, KB cells were plated at 10,000 cells per well onto a 96-well microtiter assay plate. KB cells were allowed to adhere to the plate by an overnight incubation at 37° C., 5% CO2. The spent media was then removed and wells designated to contain folic acid competitor received assay medium containing folic acid, while wells not designated to have folic acid competitor received assay medium only. The plate was incubated for 30 min at 37° C., 5% CO2 before the assay media was aspirated and plate washed with assay media. Free MMAE, 3xMMAE-XTEN and 3xFA(γ),3xMMAE-XTEN in the presence or absence of folic acid competitor was then added at an appropriate range of doses. The plate was then further incubated for 2-4 h at 37° C., 5% CO2. Media was then removed, the plate washed and fresh media introduced and the plate was allowed to incubate for an additional 48-72 h. After the appropriate incubation period, CellTiter-Glo reagent was added and the plate was read on a luminometer. The IC50 of each test article was determined using a 4 parameter logistic curve fit using GraphPad Prism.

Results: Free MMAE drug moiety showed highly potent killing of KB cells, with an IC50 of 0.8 nM, while 3 copies of MMAE conjugated to non-targeting XTEN resulted in at least a 3 log reduction in cell killing (IC50>1,000 nM). Significantly, the addition of 3 copies of folate targeting domains to the 3xMMAE-XTEN conjugate restored the cell killing, with an IC 50 of 4.2 nM; a level of activity close to that observed for free MMAE. Of equal importance, the introduction of folic acid as a competitor to the targeted conjugate impaired the observed cell killing activity of 3xFA(γ),3xMMAE-XTEN on the KB cell line. This reduction from potent cell killing of the folate-XTEN drug conjugate (from 4.2 nM to >1,000 nM) supports the conclusion that the detected cell toxicity was, under the experimental conditions, facilitated by use of the folate as the targeting mechanism for the drug conjugate against the KB cell line.

### Example 34

### In Vitro Cell-Based Evaluation of XTEN1-PCM-TM1-XTEN2

An experiment was performed to test the ability of an XTEN-conjugate composition comprising components including a folate targeting moiety (TM), a peptidyl cleavage sequence (PCM), and a cytotoxic drug, all components being linked to an XTEN in a designed configuration, in order to exhibit a differential degree in an in vitro assay of cytotoxicity on a mammalian cell in the presence or absence of a protease capable of cleaving the composition and releasing the components. In the experiment, the test XTEN-conjugate composition was conjugate #2, consisting of XTEN864 as XTEN1; PLGLAG (SEQ ID NO: 4) as the sequence of the PCM, folate as the TM, MMAF as the cytotoxic drug which was configured as XTEN432-3xMMAF using a second XTEN (XTEN2). As corresponding controls, conjugates not containing XTEN1 (conjugate #385; PLGLAG-1xfolate-XTEN432-3xMMAF (“PLGLAG” disclosed as SEQ ID NO: 4)) or XTEN1-PCM (conjugate #386; 1xfolate-XTEN432-3xMMAF) were included in the analysis. All 3 conjugates were tested in a CellTiter-Glo anti-proliferation assay utilizing folate receptor-positive human KB cells.

Briefly, KB cells were grown in folate-free RPMI plus 10% heat-inactivated fetal calf serum and were plated at 1×10⁴ into each well of a 96-well microtiter plate. The KB cells were allowed to attach to the plate by an overnight incubation at 37° C., 5% CO2. Conjugate #2, #386 and #385 with and without MMP-9 treatment were introduced in a dose range in duplicates and the plate was incubated for 3 days at 37° C., 5% CO2. After the appropriate incubation period, CellTiter-Glo reagent was added to each well, mixed for 2 minutes on an orbital shaker. The plate was then centrifuged at 90 x g and incubated at room temperature for an additional 10 minutes to stabilize the luminescent signal Luminescence signals were then read on a luminometer and an IC₅₀s (half maximal inhibitory concentration) was calculated with GraphPad Prism software.

Results. The results, shown in Table 37, demonstrated that conjugate #386 showed potent cytotoxic activity with an IC₅₀ of 0.54 nM. As conjugate #386 does not contain the PCM, treatment with MMP-9 did not affect its in vitro activity (IC₅₀ of 0.73 nM). No change in cytotoxic activity was observed between MMP-9-treated and MMP-9-untreated conjugate #385; a construct containing PCM but without XTEN1 (XTEN864) for a shielding effect (IC₅₀ of 0.94 nM and 0.75 nM respectively). Significantly, while MMP-9 treated conjugate #2 exhibited in vitro cytotoxic activities equivalent to that of conjugate #386; conjugate #2 not treated with MMP-9 was 8-fold less active. The data suggest that under the experimental conditions, the XTEN imparts a shielding effect against proteolysis by MMP-9, and that the construct with a PCM capable of being cleaved by a protease is capable of causing an enhanced degree of cytotoxicity when the protease is present.

TABLE 37
Cytotoxicity of MMP-9 treated and untreated conjugates

| Conjugate # | MMP-9 treated | IC₅₀ (nM) |
|---|---|---|
| 386 | No | 0.54 |
| 386 | Yes | 0.73 |
| 385 | No | 0.75 |
| 385 | Yes | 0.94 |
| 2 | No | 6.88 |
| 2 | Yes | 0.82 |

### Example 35

### In Vitro Cytotoxicity and Specificity Evaluation of FA-XTEN432-3xMMAF, XTEN432-3xMMAF, FA-XTEN864-3xMMAF and XTEN864-3xMMAF Conjugates

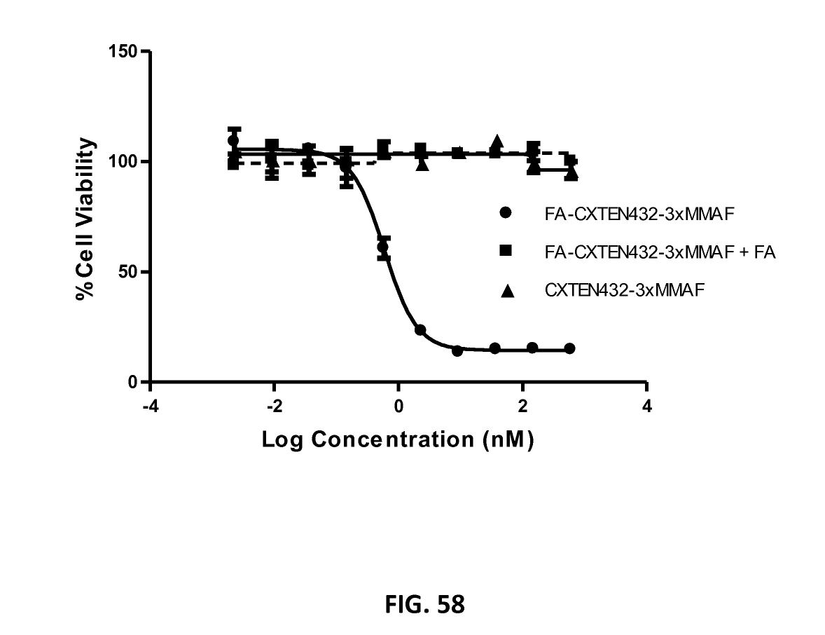

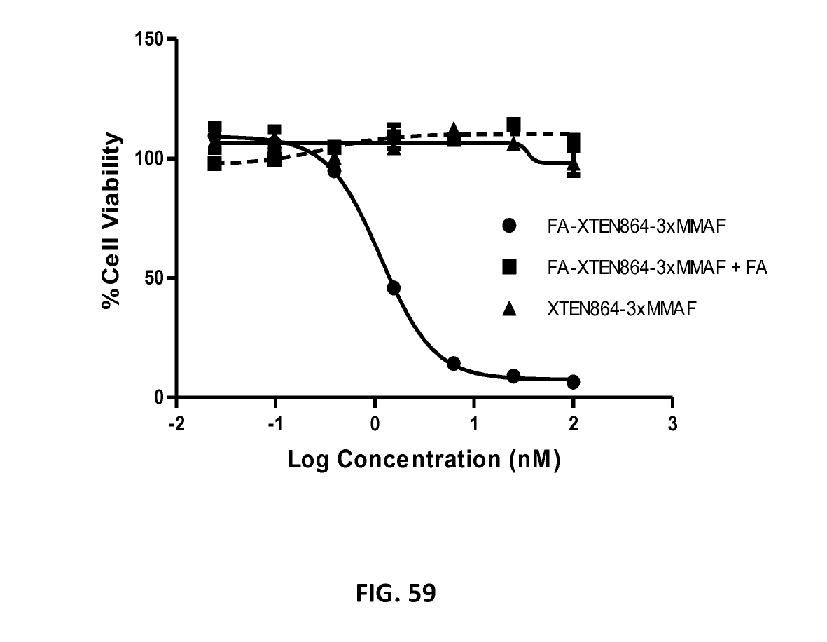

The cytotoxic activity of four conjugate constructs; two having a folic acid (FA) targeting moiety and two without, with all four having three molecules of MMAF conjugated to XTEN_432 or XTEN_864, were compared for the ability to effect cytotoxicity in an in vitro assay. FA-XTEN432-3xMMAF, XTEN432-3xMMAF, FA-XTEN864-3xMMAF and XTEN864-3xMMAF were evaluated in a CellTiter-Glo cell viability assay using the folate receptor positive KB cell line in the presence and absence of free folic acid (FA) as competitor (FIG. 58 and FIG. 59). As cell culture media contain high folic acid content, the KB cells were grown and the assay performed in folic acid-free RPMI plus 10% heat-inactivated fetal calf serum (FCS). Briefly, KB cells were plated at 1×104 into each well of a 96-well microtiter plate and allowed to attach to the plate by an overnight incubation at 37° C., 5% CO2. The FA-XTEN432-3xMMAF and FA-XTEN864-3xMMAF conjugates, with and without folic acid treatment, as well as XTEN432-3xMMAF and XTEN864-3xMMAF conjugates were all introduced into the plate in a 4 fold serial dilution dose range of 600 to 0.002 nM for the XTEN432 series or 100 to 0.02 nM for the XTEN864 series (in duplicate) and plate was incubated for 3 days at 37° C., 5% CO2. After the appropriate incubation period, CellTiter-Glo reagent was added to each well, and the plate was mixed for 2 min on an orbital shaker. The plate was then centrifuged at 90×g and incubated at room temperature for an additional 10 min to stabilize the luminescent signal. Luminescence signals were then read on a luminometer and an IC50s (half maximal inhibitory concentration) was calculated with GraphPad Prism software.

Results: As shown in FIG. 58, dosed at equimolar concentration, FA-XTEN432-3xMMAF, with the folate targeting moiety, showed highly potent killing with IC₅₀ of 0.6 nM while the XTEN432-3xMMAF conjugate construct bearing no targeting moiety was drastically less effective by at least 3 logs, yielding minimal cytotoxicity (IC₅₀>600 nM). Importantly, the addition of folic acid as competitor impaired the observed cell killing activity of FA-XTEN432-3xMMAF on KB cells (IC₅₀>600 nM), demonstrating the specificity of the folate receptor-mediated cytotoxicity (FIG. 58). Similar results were obtained with the XTEN864-containing conjugates. FA-XTEN864-3xMMAF exhibited potent cytotoxicity (IC₅₀ of 1.2 nM) that was inhibited by folic acid (IC₅₀>100 nM), while the corresponding non-targeted XTEN864-3xMMAF conjugate did not possess any significant in vitro cytotoxic activity (IC₅₀>100 nM) (FIG. 59).

Conclusions:The data suggest that under the experimental conditions, the folate moiety imparts a folate receptor-mediated targeting effect that enhanced the cytotoxic activity of the XTEN-drug conjugates.

### Example 36

### In Vivo Efficacy and Safety Evaluation of FA-XTEN432-3xMMAF, XTEN432-3xMMAF, FA-XTEN864-3xMMAF and XTEN864-3xMMAF Conjugates

Folate-XTEN-drug conjugates are intended for targeted delivery of the toxin component to folate receptor positive tumor cells. Table 18 describes examples of tumor lines that can be used in a xenograft study. As an example, the folate receptor positive human KB cervical cell line was used to determine the in vivo efficacy and safety of the folate-XTEN-drug constructs. Prior to beginning the in vivo efficacy and safety study, two studies in female nu/nu mice were carried out to determine: (1) pharmacokinetics of targeted FA-XTEN432-3xMMAF and the corresponding non-targeting XTEN432-3xMMAF molecules; and (2) the maximum tolerated dose (MTD) of FA-XTEN432-3xMMAF conjugate. Results from both studies contributed to the final design of the efficacy & safety study for appropriate dose level and dosing frequency to be used. As normal rodent chow contains a high concentration of folic acid (6 mg/kg chow), all mice used in these studies were maintained on a folate deficient diet for 2 weeks prior to study initiation and for the duration of the study to reduce serum folate concentration to level representing human physiological range.

1) Pharmacokinetics of FA-XTEN432-3xMMAF and XTEN432-3xMMAF in Female nu/nu Mice on a Low-Folate Diet

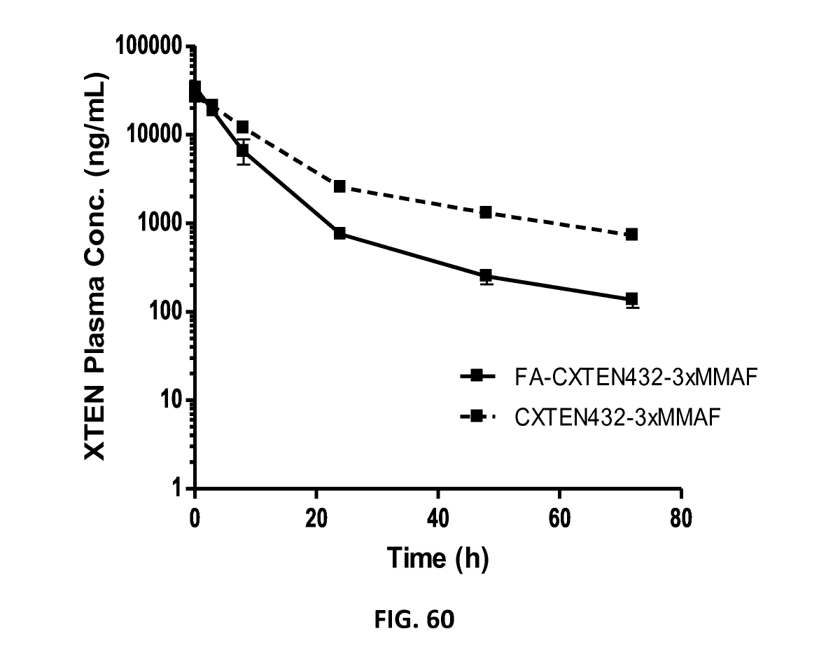

Six female nu/nu mice were intravenously injected with a single dose of 2 mg/kg of the FA-XTEN432-3xMMAF, and six female nu/nu mice were intravenously injected with a single dose of 2 mg/kg of XTEN432-3xMMAF. Blood was drawn and processed into plasma at the denoted time points of pre-dose, 10 min, 3 h, 8 h, 1 d, 2 d and 3 d post-dose for both groups. The amount of FA-XTEN432-3xMMAF and XTEN432-3xMMAF present in the various plasma samples were quantitated using a sandwich ELISA with an anti-XTEN antibody. The elimination half-life of FA-XTEN432-3xMMAF was estimated to be 9.1 h and XTEN432-3xMMAF to be 13.7 h (FIG. 60) using PK Solutions v2 software.

2) MTD of FA-XTEN432-3xMMAF in Female nu/nu Mice on a Low-Folate Diet

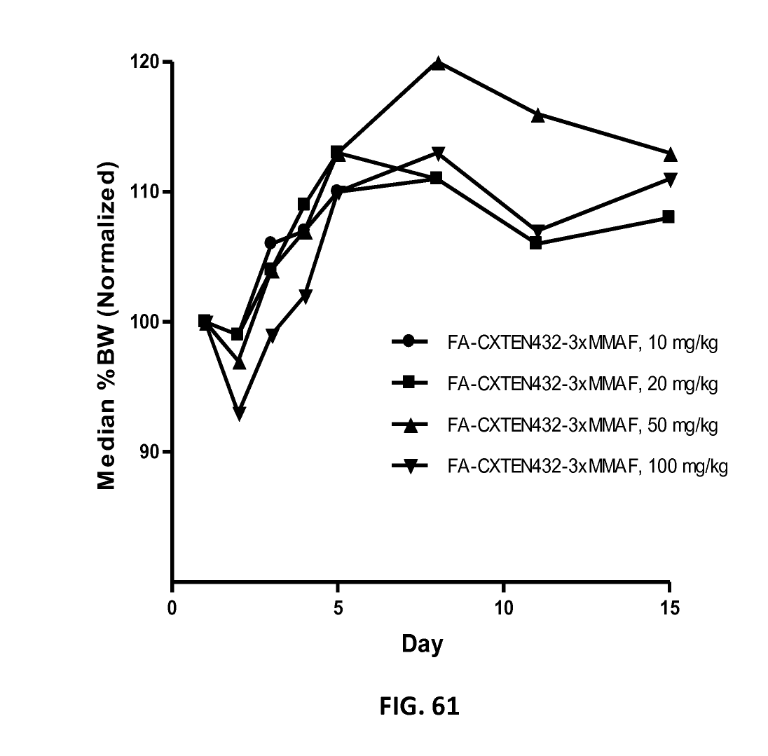

The single dose MTD experiment was carried out with 5 female nu/nu mice per group, evaluating the IV administration of FA-XTEN432-3xMMAF at 10, 20, 50 and 100 mg/kg (molar equivalent of 0.5, 1, 2.5 and 5 mg/kg MMAF respectively). As a measure of gross toxicity, the body weight of each animal was monitored daily for the first week and then twice per week until the study endpoint of 15 days. In addition, any death and clinical signs of piloerection, hunched behavior patterns, respiratory pattern, tremors, convulsions, prostration, self-mutilation and dehydration were recorded. The XTEN-drug conjugate was considered to have acceptable toxicity when the group mean body weight loss was <20% during the study and not more than 10% occurrence in treatment-related death among treated animals The MTD was set as the highest dose with acceptable toxicity. All regimens of FA-XTEN432-3xMMAF were acceptably tolerated with minimal body weight loss of 1 to 6% (FIG. 61); and exhibited no clinical signs consistent with drug-related side effects. Hence, the MTD of FA-XTEN432-3xMMAF was determined to be >100 mg/kg.

3) Efficacy and Safety Analysis of Folate-XTEN-Drug Conjugates

Based on the PK and MTD results obtained, above, the following study design was adopted for the efficacy and safety assessment of folate-XTEN-drug conjugates. To reduce folate content, the folate receptor positive KB cells to be implanted into nu/nu mice were first grown in folic acid-free cell culture media containing 10% heat-inactivated fetal calf serum with no antibiotics. Similarly, to reduce serum folate concentration, mice used in the xenograft studies were maintained on a folate deficient diet for 2 weeks prior to tumor implantation and for the duration of the study. Briefly, 3×10⁶ KB cells were injected subcutaneously in the flank of nu/nu mice and allowed to form tumors, the size of which was measured with calipers and the volume calculated as 0.5×L×W², where L=measurement of longest axis in millimeters and W=measurement of axis perpendicular to L in millimeters. Mice bearing KB tumor sizes of 75-162 mm³ were randomized into 7 groups of 8 animals per group and administered intravenously with the respective XTEN-drug conjugates, 3×per week for 1 week according to Table 38.

TABLE 38
Experimental dose schedule

| | | | | | Total |
|---|---|---|---|---|---|
| | | | | Dose | MMAF |
| | | | Dose/ | per | equivalent |
| | | | injection | week | per week |
| Group | N | Agent | (mg/kg) | (mg/kg) | (mg/kg) |
| 1 | 8 | PBS | 0 | 0 | 0 |
| 2 | 8 | FA-XTEN432-3xMMAF | 6.7 | 20 | 1 |
| 3 | 8 | FA-XTEN432-3xMMAF | 26.4 | 79 | 4 |
| 4 | 8 | XTEN432-3xMMAF | 26.4 | 77 | 4 |
| 5 | 8 | FA-XTEN864-3xMMAF | 12.7 | 38 | 1 |
| 6 | 8 | FA-XTEN864-3xMMAF | 50 | 151 | 4 |
| 7 | 8 | XTEN864-3xMMAF | 50 | 149 | 4 |

Cessation or regression of tumor growth was determined through caliper measurement twice per week until the study endpoint. The endpoint of the study was defined as tumor volume of 2,000 mm³ or 60 days, whichever comes first. Animals were scored as being partial regressions (PR) when tumor volume was determined to be <50% of its day 1 volume for three consecutive measurements during the course of the study, and >13.5 mm³ for one or more of these measurements. Animals were considered to be complete regression (CR) when the tumor volume was <13.5 mm³ for three consecutive measurements during the study. Any animal with a complete regression at the end of the study was further classified as tumor free survivor (TFS). To assess gross toxicity, animals were monitored individually for body weight on a daily basis for the first week and then twice per week till study endpoint. Any clinical signs of distress, deaths or adverse events were also recorded.

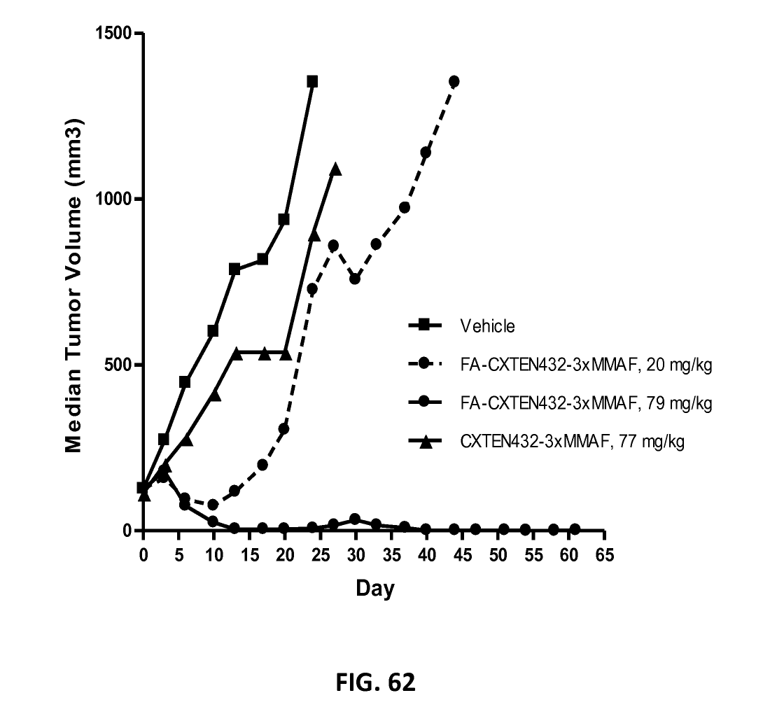

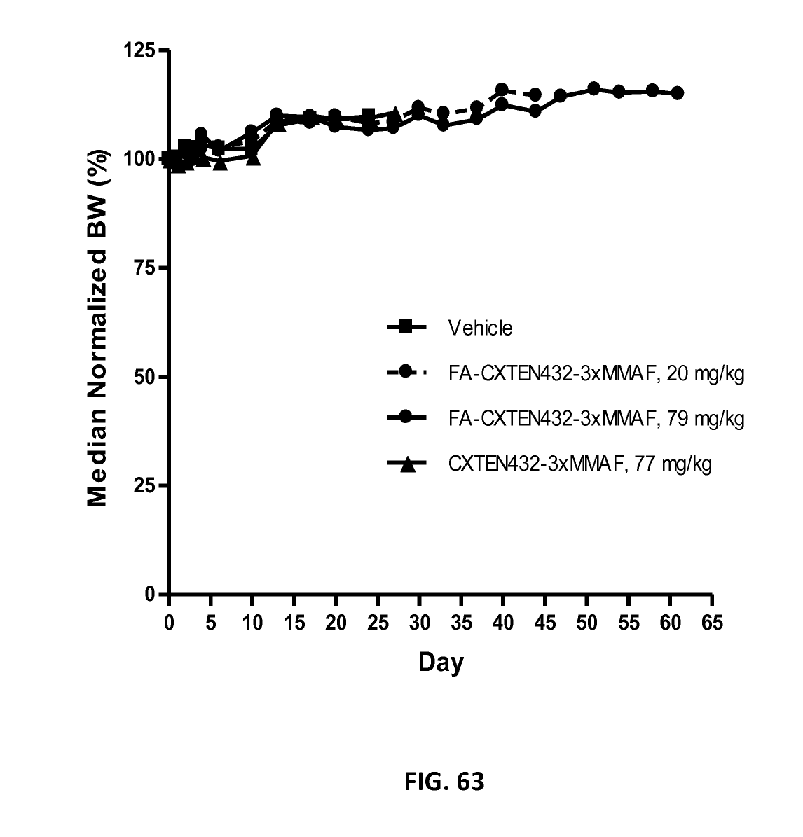

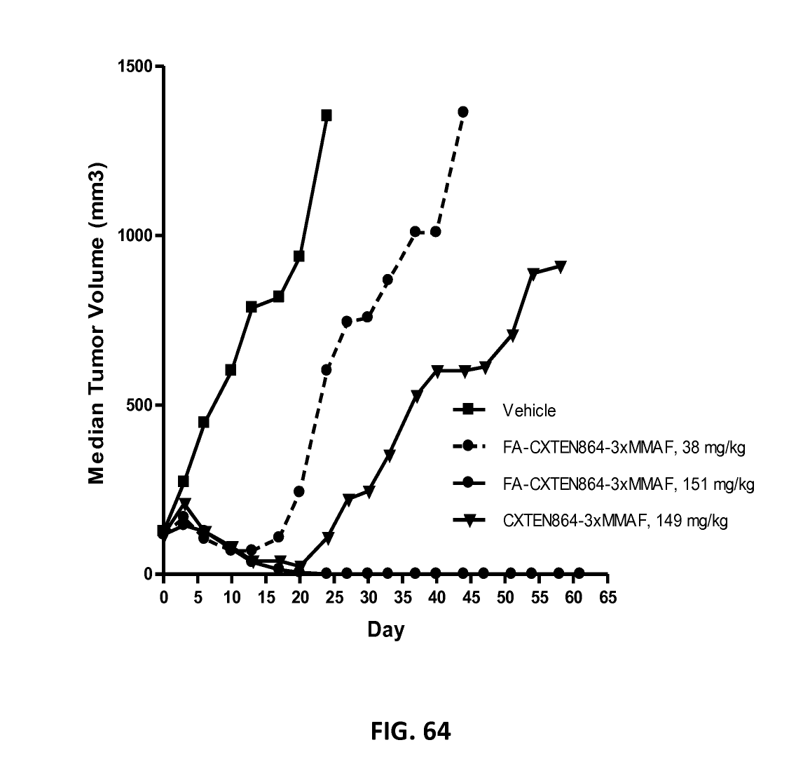

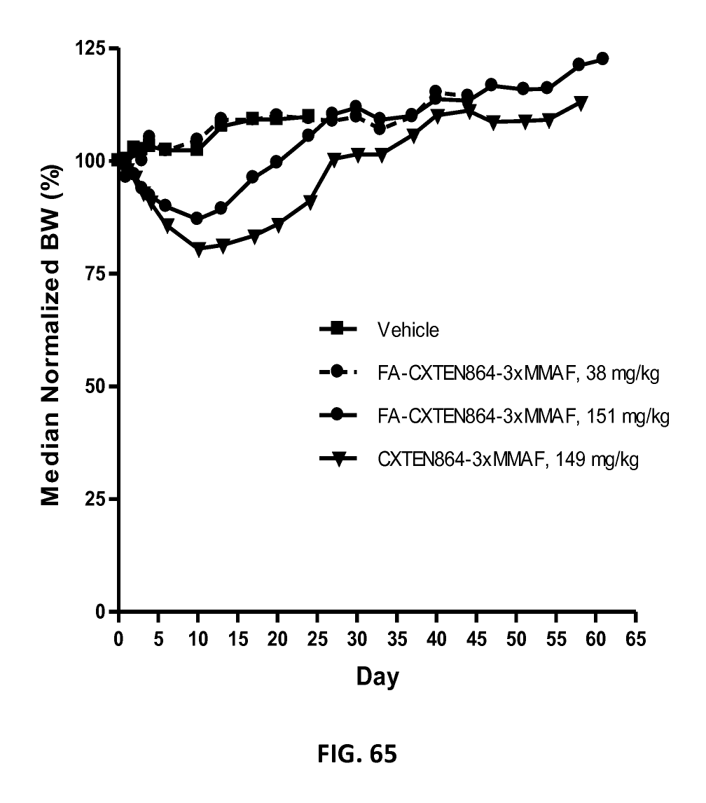

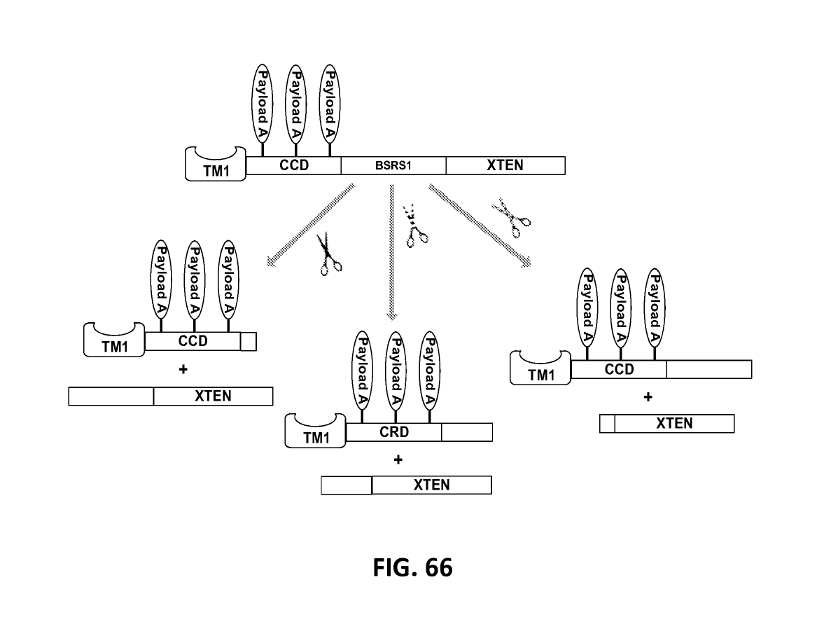

Results: As shown in FIGS. 62 and 63, FA-XTEN432-3xMMAF administered at 79 mg/kg was found to be efficacious and well-tolerated yielding 2 PR, 6CR and 5TFS with no visible weight loss. In contrast, the XTEN432-3xMMAF injected group exhibited aggressive tumor progression with no responders (FIG. 62). At 38 mg/kg, FA-XTEN864-3xMMAF yielded 1 PR with no significant loss in body weight (FIGS. 64 and 65). However, at 151 mg/kg, the FA-XTEN864-3xMMAF conjugate was highly efficacious with 8 TFS (FIG. 64) and acceptable toxicity with a maximum body weight loss of 12% by day 11 (FIG. 65). The non-targeted XTEN864-3xMMAF also caused tumor regression to some extent with 2 PR, 2 CR and 1 TFS (FIG. 64). This group, however, had the highest toxicity among all conjugates tested with a maximum loss in body weight of 20% by day 11 (FIG. 65). All animals in FA-XTEN864-3xMMAF and XTEN864-3xMMAF group regained body weight and recovered well over time (FIG. 65).

Conclusions: The data strongly suggest that within the context of the KB xenograft model, presence or absence of the folate targeting moiety, as well as XTEN length, have a significant impact on the performance of XTEN-drug conjugates. Drug conjugates bearing targeting moiety are more efficacious than their non-targeting counterparts; and XTEN864-containing constructs were more effective than XTEN432-containing constructs.

### Example 37

### Pharmacokinetics and Bio-Distribution Analysis of Anti-HER2scFv-XTEN432, XTEN432, anti-HER2scFv-XTEN864 and XTEN864 in Female Athymic Nude Mice Bearing Human BT474 Breast Carcinoma

The pharmacokinetic and biodistribution properties of targeted XTEN and separate XTEN molecules were analyzed in female athymic nude mice bearing human BT474 breast carcinoma. To enable the concurrent evaluation of anti-HER2scFv-XTEN432, XTEN432, anti-HER2scFv-XTEN864 and XTEN864 in the same mouse and to minimize mouse-to-mouse variation, anti-HER2scFv-XTEN432, XTEN432, anti-HER2scFv-XTEN864 and XTEN864 were each labeled with an orthogonal rare lanthanide earth ion via the DOTA (1,4,7,10-tetraazacyclododecane-1,4,7,10-tetraacetic acid) chelator to yield the respective molecules: anti-HER2scFv-XTEN432-DOTA-holmium (Ho), XTEN432-DOTA-Thulium (Tm), anti-HER2scFv-XTEN864-DOTA-Terbium (Tb) and XTEN864-DOTA_Lutetium (Lu). All 4 labeled proteins were then mixed at equimolar concentration for administration at 2 targeted dose levels of 26 nmol/kg and 460 nmol/kg. The 26 nmol/kg dose was selected so as not to cause HER2 receptor saturation and to avoid competition between anti-HER2scFv-XTEN432-DOTA-Ho and anti-HER2scFv-XTEN864-DOTA-Tb for available HER2 binding sites on the BT474 tumor cells. The 460 nmol/kg dose on the other hand was likely to cause HER2 receptor saturation and competition between both HER2 targeting XTENylated proteins.

Eighteen female athymic nude mice were injected subcutaneously in the flank with 1×10⁷ BT474 (ATCC cat #HTB-20) human breast cancer cells. The grafted tumors were monitored as their volumes approached the target range of 250-350 mm³, the size of which was measured with calipers and the volume calculated as 0.5×(L×W²) where L=length and W=width, in millimeters (mm) of the tumor. A month after cells implant, designated as Day 0, 12 out of the 18 mice bearing appropriate BT474 tumor size were sorted into 4 groups of 3 animals per group. At Day 0, individual tumor volume of the 12 mice ranged from 152 to 381 mm³ and group mean tumor volumes were 256 to 257 mm³ Treatment was initiated on Day 0 with the intravenous administration of 26 nmol/kg of anti-HER2scFv-XTEN432-DOTA-Ho, XTEN432-DOTA-Tm, anti-HER2scFv-XTEN864-DOTA-Tb and XTEN864-DOTA-Lu mixture to 6 mice with established BT474 xenograft. The other 6 mice were injected with 460 nmol/kg of the protein mixture.

For pharmacokinetics analysis, blood were drawn into lithium heparinized tubes and processed into plasma at defined time points of pre-dose, 3 h, 8 h, 1 d, 2 d and 3 d post-dose for both dose groups. The amount of anti-HER2scFv-XTEN432-DOTA-Ho, XTEN432-DOTA-Tm, anti-HER2scFv-XTEN864-DOTA-Tb and XTEN864-DOTA-Lu present in the various plasma samples were quantitated for the respective rare-earth metal using Inductively Coupled Plasma Mass Spectrometry (ICP-MS). The elimination half-life for each labeled protein administered at 26 nmol/kg and 460 nmol/kg was determined using GraphPad Prism (San Diego).

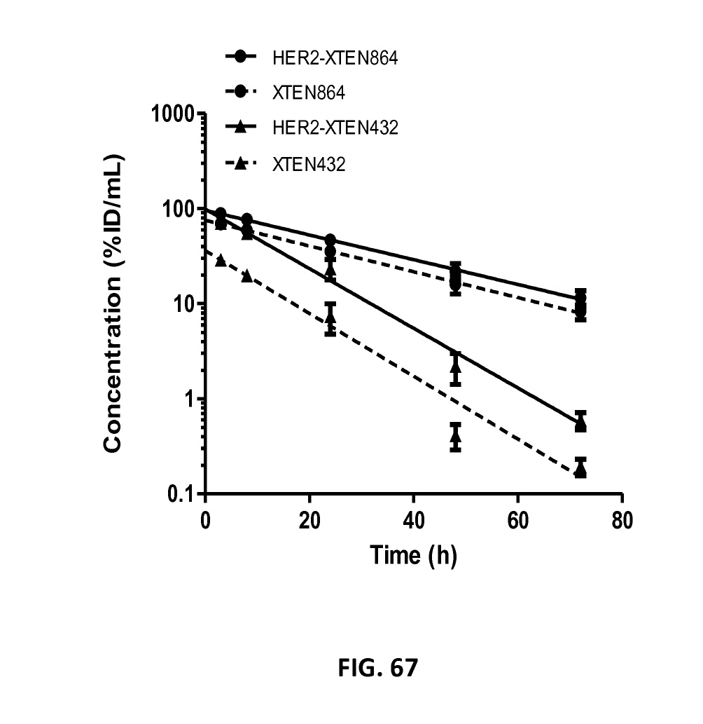

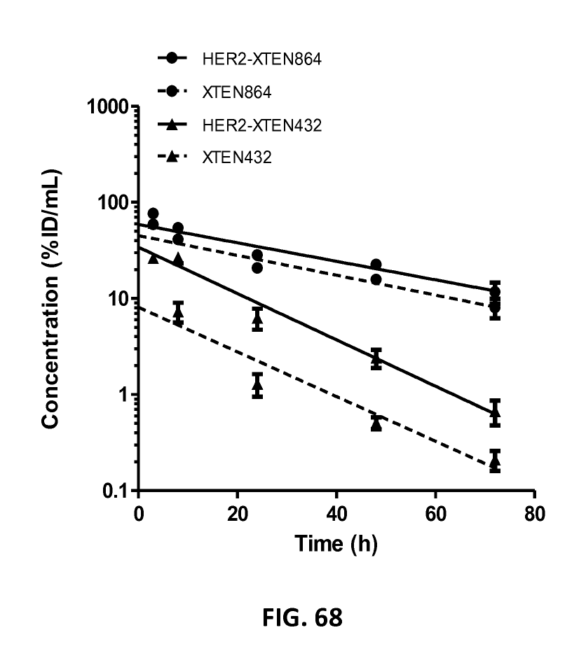

The T_{1/2} for anti-HER2scFv-XTEN432-DOTA-Ho was estimated to be 9.6 to 12.5 h; 9.2 to 13.0 h for XTEN432-DOTA-Tm; 23.1 to 31.2 h for anti-HER2scFv-XTEN864-DOTA-Tb and 22.2 to 29.2 h for XTEN864-DOTA-Lu (FIGS. 67 and 68). As expected, XTEN length has a critical influence on half-life; with the longer 864 amino acid XTEN providing a T_{1/2} of 22-31 h versus the shorter 432 amino acid XTEN with a T_{1/2} of 9-13 h. The presence of the anti-HER2 targeting scFv, on the other hand, seemed to have little effect on plasma exposure.

For tissue bio-distribution analysis, 3 mice from each dose level were sacrificed at 24 h and another 3 mice at 72 h. At the respective terminal endpoint, tumor, heart, kidneys, liver, lungs, spleen, pancreas and brain were harvested, rinsed in PBS, blotted dry, weigh and snap frozen. The amount of anti-HER2scFv-XTEN432-DOTA-Ho, XTEN432-DOTA-Tm, anti-HER2scFv-XTEN864-DOTA-Tb and XTEN864-DOTA-Lu present in the various tissues were quantitated by each rare-earth metal using ICP-MS and data expressed as percent injected dose per g tissue (% ID/g).

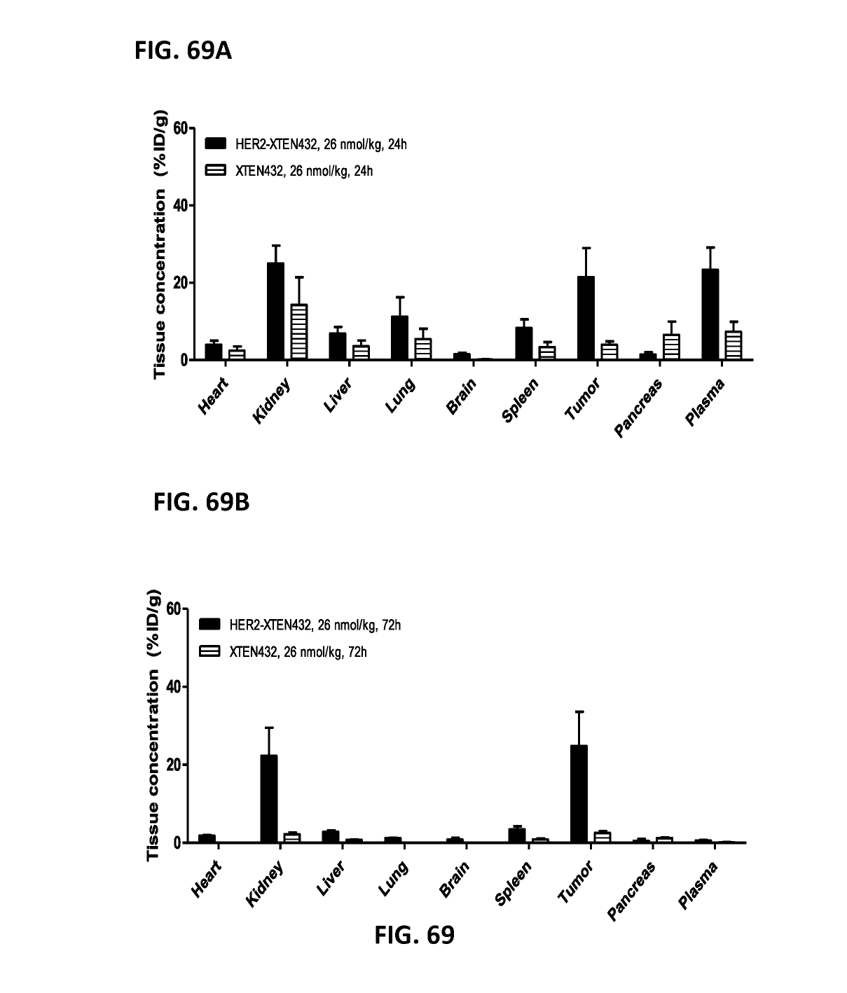

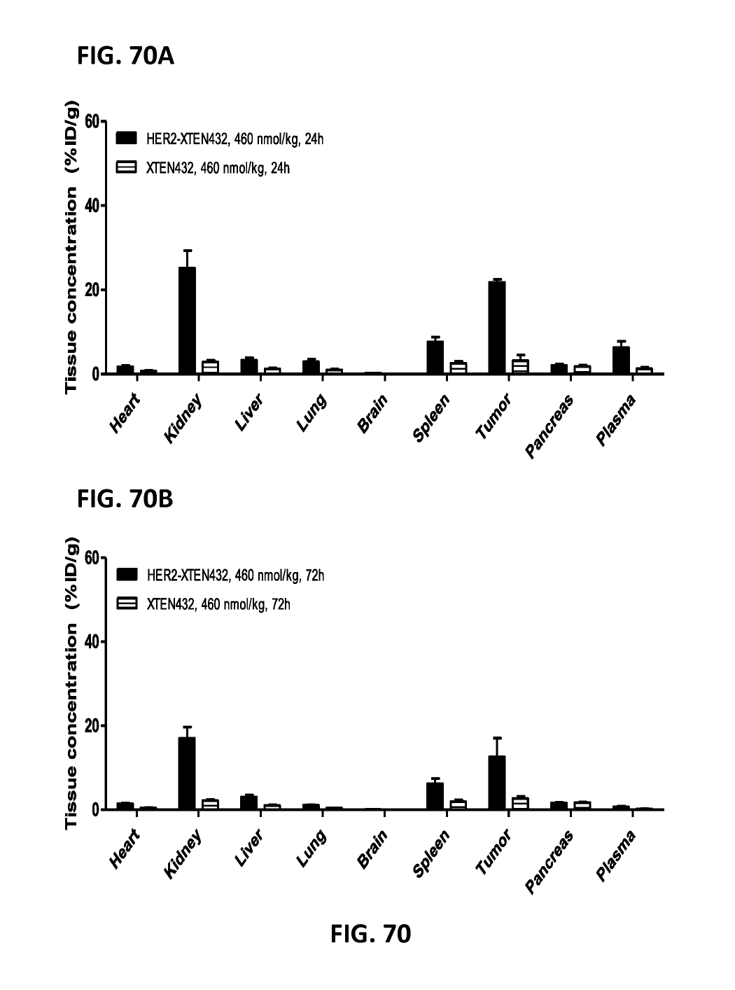

At 26 nmol/kg, a concentration decay of both anti-HER2scFv-XTEN432-DOTA-Ho and XTEN432-DOTA-Tm in plasma and healthy tissues was observed; with the exception of considerable accumulation of anti-HER2scFv-XTEN432-DOTA-Ho in the liver and tumor (FIGS. 69A and 69B) (It was noteworthy that the brain had minimal detectable level of anti-HER2scFv-XTEN432-DOTA-Ho and XTEN432-DOTA-Tm, suggesting that XTENylated proteins did not pass through the blood brain barrier and localized in the brain.). It is the desired outcome that HER2 targeting resulted in effective (25±15% ID/g) and sustained accumulation in the tumor as compared to the non-targeted construct (3±1% ID/g). While it is currently unknown why anti-HER2scFv-XTEN432-DOTA-Ho accumulated in the kidney, a plausible explanation would be that anti-HER2scFv-XTEN432-DOTA-Ho was cleared via the kidney and thus subjected to tubular reabsorption due to the presence of the anti-HER2 scFv moiety. This was supported by the observation that the non-HER2 targeted XTEN432-DOTA-Tm when cleared was not reabsorbed or accumulated in the kidney. The same bio-distribution pattern was observed at the 460 nmol/kg dose level, albeit at a lower quantitative level due possibly to some competition for the HER2 binding sites from the anti-HER2scFv-XTEN864-DOTA-Tb construct in the injected mixture (FIGS. 70A and 70B)

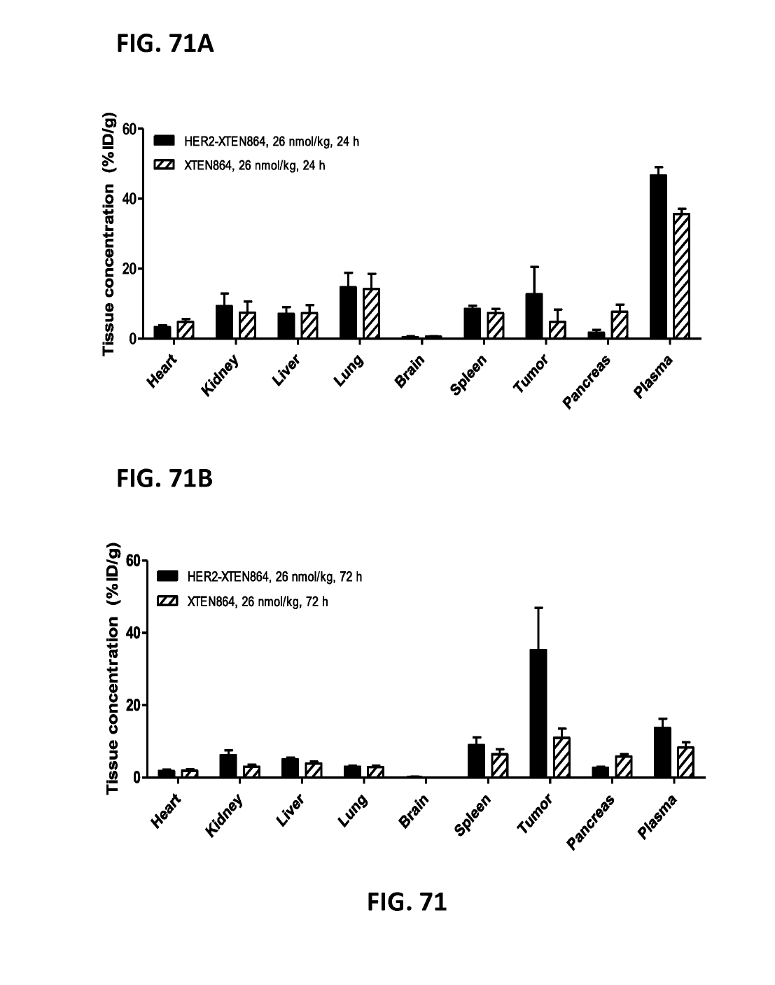

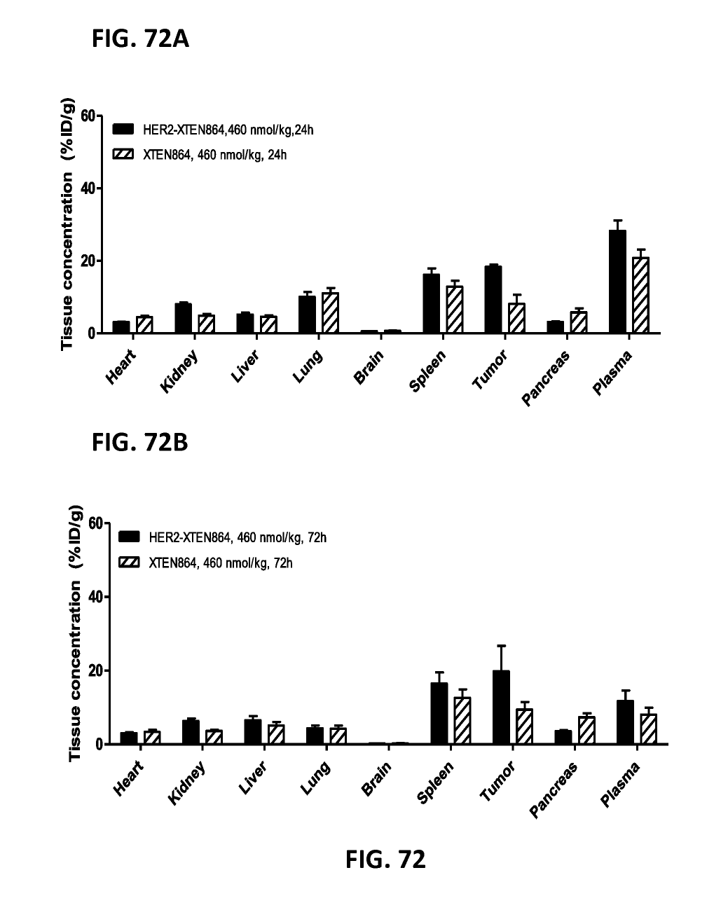

At 26 nmol/kg, a concentration decay of anti-HER2scFv-XTEN864-DOTA-Tb and XTEN864-DOTA-Lu in plasma and healthy tissues were observed, with the exception of significant accumulation of anti-HER2scFv-XTEN864-DOTA-Tb and to some extend XTEN864-DOTA-Lu in the tumor (FIGS. 71A and 71B). It is the desired expectation that anti-HER2scFv-XTEN864-DOTA-Tb was demonstrated not only to accumulate strongly in the tumor (35±20% ID/g) but also preferentially better than the non-targeted XTEN864-DOTA-Lu (11±4% ID/g). It is believed that the XTEN864-DOTA-Lu accumulated in the tumor due to the enhanced permeability and retention effect because of its large size. In contrast to the anti-HER2scFv-XTEN432-DOTA-Ho conjugate, no kidney accumulation of anti-HER2scFv-XTEN864-DOTA-Tb was observed. This can be accounted for by anti-HER2scFv-XTEN864-DOTA-Tb exceeding the size for kidney clearance. The same bio-distribution profiles for anti-HER2scFv-XTEN864-DOTA-Tb and XTEN864-DOTA-Lu were observed at 460 nmol/kg dose, not with standing at a lower quantitative level for the anti-HER2scFv-XTEN864-DOTA-Tb construct due possibly to some competition for the HER2 binding sites from the anti-HER2scFv-XTEN432-DOTA-Ho construct (FIGS. 72A and 72B). It is currently unknown why there was some apparent accumulation of both anti-HER2scFv-XTEN864-DOTA-Tb and XTEN864-DOTA-Lu in the spleen at 72 h.

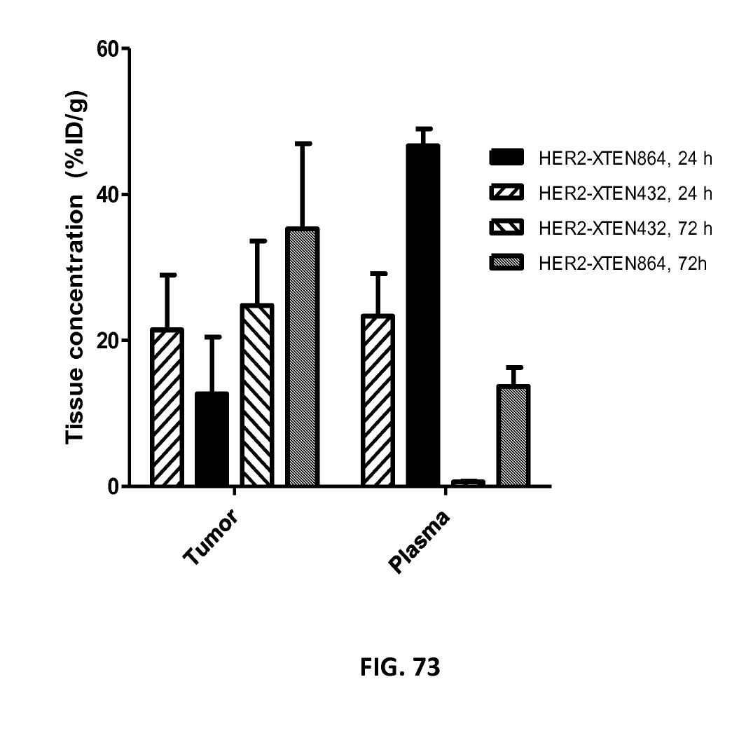

Further, it is also in line with expectation that the smaller anti-HER2scFv-XTEN432-DOTA-Ho accumulated faster in the tumor than the larger anti-HER2scFv-XTEN864-DOTA-Tb at 24 h. However, over time at 72 h, due to its longer circulating half-life, considerably more anti-HER2scFv-XTEN864-DOTA-Tb was found to accumulate in the tumor than anti-HER2scFv-XTEN432-DOTA-Ho (FIG. 73).

### Example 38

### Efficacy and Safety Analysis of Anti-HER2scFv-3xMMAE-XTEN720 in Female Athymic Nude Mice Bearing Human BT474 Breast Cancer Cells

The following study design was utilized for the efficacy and safety assessment of anti-HER2scFv-3xMMAE-XTEN720 drug conjugate in the BT474 xenograft model in athymic nude mice. Briefly, 1×10⁷ BT474 cells were injected subcutaneously in the flank of female athymic nude mice and allowed to form tumors, the size of which was measured with calipers and the volume calculated as 0.5×(L×W²), where L=length and W=width, in millimeters (mm) of the tumor. Twenty-nine days after cells implant, designated as Day 0, mice bearing targeted tumor size of 100-200 mm³ were sorted into 4 groups of 8 animals per group. At Day 0, individual tumor volume of the mice enrolled in the study ranged from 107 to 278 mm³ and group mean tumor volumes were 183 to 185 mm³. Treatment was initiated on Day 0 with the intravenous administration of PBS vehicle control, 30, 100 and 300 nmol/kg of the anti-HER2scFv-3xMMAE-XTEN720 drug conjugate.

Cessation or regression of tumor growth was determined through caliper measurement three times per week till study endpoint. The endpoint of the study was defined as tumor volume of 800 mm³ or 30 days, whichever comes first. Percent tumor growth inhibition (% TGI) was calculated for each treatment group with the following formula: ((Mean tumor volume of vehicle control−Mean tumor volume of HER2 conjugate)/mean tumor volume of vehicle control)×100. Treatment group with % TGI 60% is considered therapeutically active. As an assessment of gross toxicity, animals were monitored individually for body weight three times per week till study endpoint. Any clinical signs of distress, deaths or adverse events were also noted.

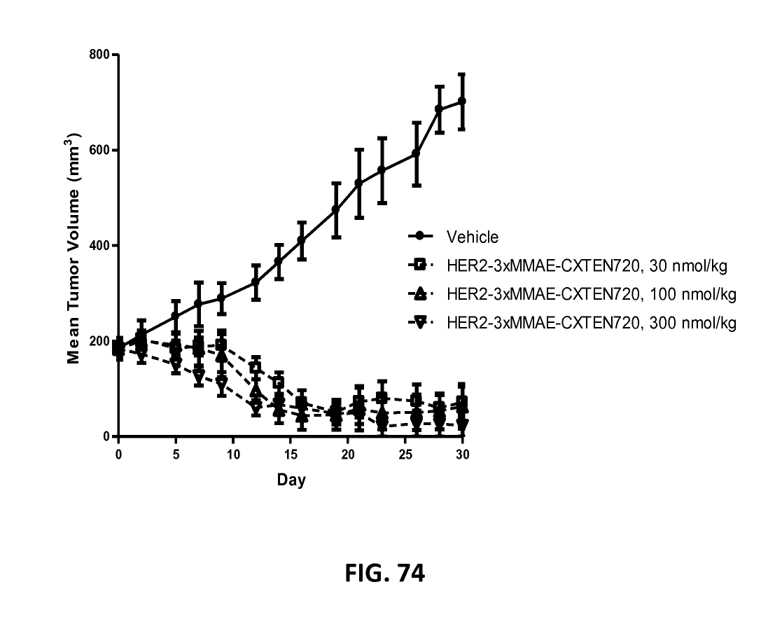

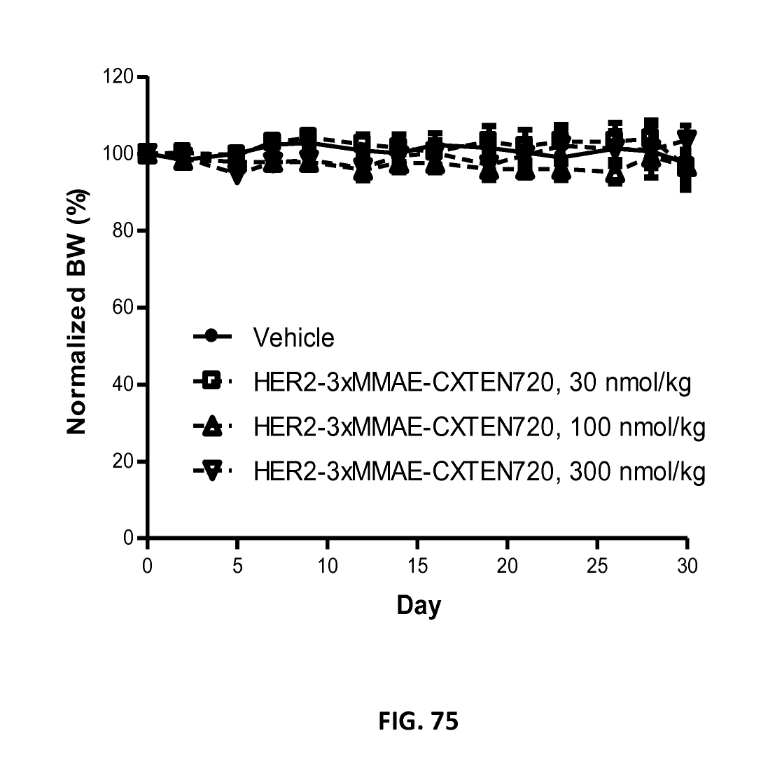

As expected, there was no tumor inhibition in BT474 tumor bearing mice administered with PBS vehicle, yielding a % TGI of zero. In contrast and as shown in FIG. 74, anti-HER2scFv-3xMMAE-XTEN720 drug conjugate was found to be efficacious at all 3 doses evaluated and was well tolerated with no visible weight loss (FIG. 75). At Day 30, the % TGI for anti-HER2scFv-3xMMAE-XTEN720 dosed at 30 nmol/kg was 90%; at 100 nmol/kg, 91% and at 300 nmol/kg, 97%; suggesting that 30 nmol/kg of drug conjugate has almost equivalent efficacy as 300 nmol/kg.

The above data strongly suggest that within the context of the BT474 xenograft model, anti-HER2scFv-3xMMAE-XTEN720 is highly efficacious and safe, with efficacy achievable at doses as low as 30 nmol/kg of drug conjugate.

### Example 39

### Pharmacokinetic Determination of PCM Containing XTEN

The pharmacokinetic properties of XTEN bearing different PCM sequences were analyzed in female athymic nude mice. PCM with RS1 (MMP-2/9), RS2 (MMP-7), RS3 (uPA) and RS4 (MMP-14) composition was first evaluated; followed by PCM with tandem protease release sites BSRS1, BSRS2 and BSRS3 in study 2. To enable the concurrent analysis of the various RS & BSRS configurations on the circulating half-life of XTEN in the same mouse as well as to minimize mouse-to-mouse variation, the respective XTEN-RS and XTEN-BSRS were each labeled with an orthogonal rare lanthanide earth ion via the DOTA (1,4,7,10-tetraazacyclododecane-1,4,7,10-tetraacetic acid) chelator to yield the corresponding molecules in support of the two studies.

In study 1, XTEN864-RS1-DOTA-holmium (Ho), XTEN864-RS2-DOTA-Terbium (Tb), XTEN864-RS3-DOTA-Thulium (Tm), XTEN864-RS4-DOTA-Europium (Eu) and control XTEN864-DOTA-Lutetium (Lu) were analyzed. Six female nu/nu mice were intravenously injected with a mixture containing 2 mg/kg each of XTEN864-RS1-DOTA-Ho, XTEN864-RS2-DOTA-Tb, XTEN864-RS3-DOTA-Tm, XTEN864-RS4-DOTA-Eu and XTEN864-DOTA-Lu. Blood was drawn and processed into plasma at the denoted time points of pre-dose, 3 h, 8 h, 1 d, 2 d, 3 d and 4 d post-dose between both groups. The amount of XTEN864-RS1-DOTA-Ho, XTEN864-RS2-DOTA-Tb, XTEN864-RS3-DOTA-Tm, XTEN864-RS4-DOTA-Eu and XTEN864-DOTA-Lu present in the various plasma samples were quantitated for the respective rare-earth metal using Inductively Coupled Plasma Mass Spectrometry (ICP-MS). The elimination half-life for each labeled protein administered at 2 mg/kg was determined using GraphPad Prism (San Diego).

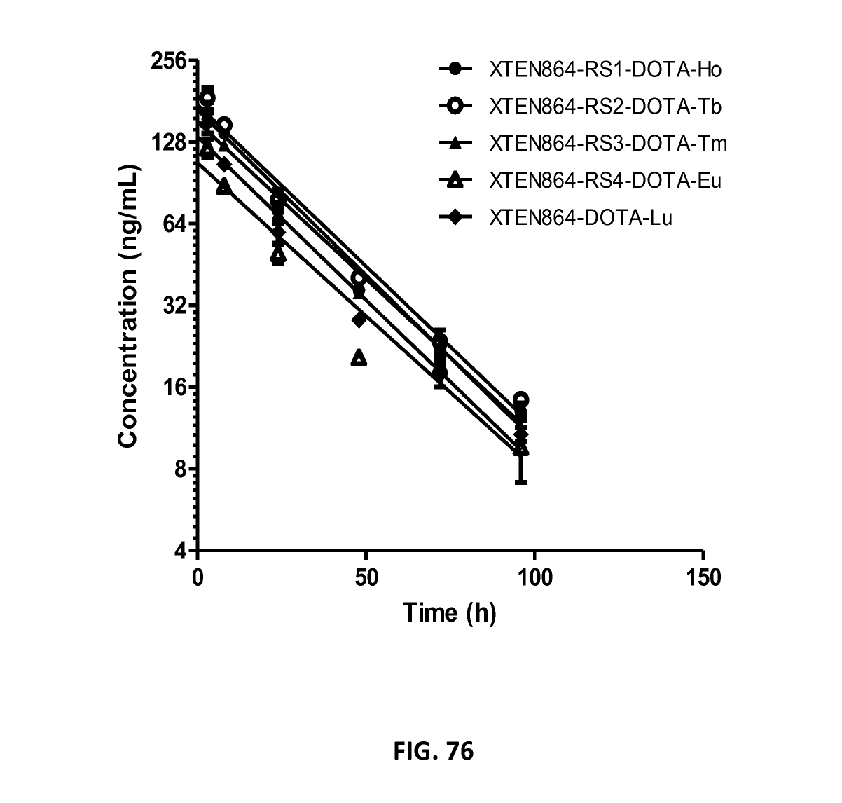

The T_{1/2} for XTEN864-RS1-DOTA-Ho was estimated to be 24.7 h, XTEN864-RS2-DOTA-Tb 25.3 h, XTEN864-RS3-DOTA-Tm 26.0 h, XTEN864-RS4-DOTA-Eu 26.7 h and XTEN864-DOTA-Lu 25.0 h (FIG.76). Results indicated that the presence of RS1, RS2, RS3 and RS4 composition on XTEN did not alter the T_{1/2} of the XTEN polymer.

In study 2, XTEN containing different tandem protease release sites were evaluated with XTEN864-BSRS1-DOTA-Tb, XTEN864-BSRS2-DOTA-Ho, XTEN864-BSRS3-DOTA-Tm, XTEN864-DOTA-Lu and XTEN144-DOTA-Eu; with XTEN864-DOTA-Lu serving as the non-B SRS containing XTEN and XTEN144-DOTA-Eu representing the cleaved shorten moiety. All 5 metal-labeled proteins were mixed at 2 mg/kg concentration and the resultant mixture intravenous administered into six female nu/nu mice. For pharmacokinetics analysis, blood were drawn into lithium heparinized tubes and processed into plasma at defined time points of pre-dose, 3 h, 8 h, 1 d, 2 d, 3 d, 4 d, 5 d, 6 d and 7 d post-dose among the six mice. The amount of XTEN864-BSRS1-DOTA-Tb, XTEN864-BSRS2-DOTA-Ho, XTEN864-BSRS3-DOTA-Tm, XTEN864-DOTA-Lu and XTEN144-DOTA-Eu present in the various plasma samples were quantitated for the respective rare-earth metal using ICP-MS. The elimination half-life for each labeled protein administered at 2 mg/kg was determined using GraphPad Prism (San Diego).

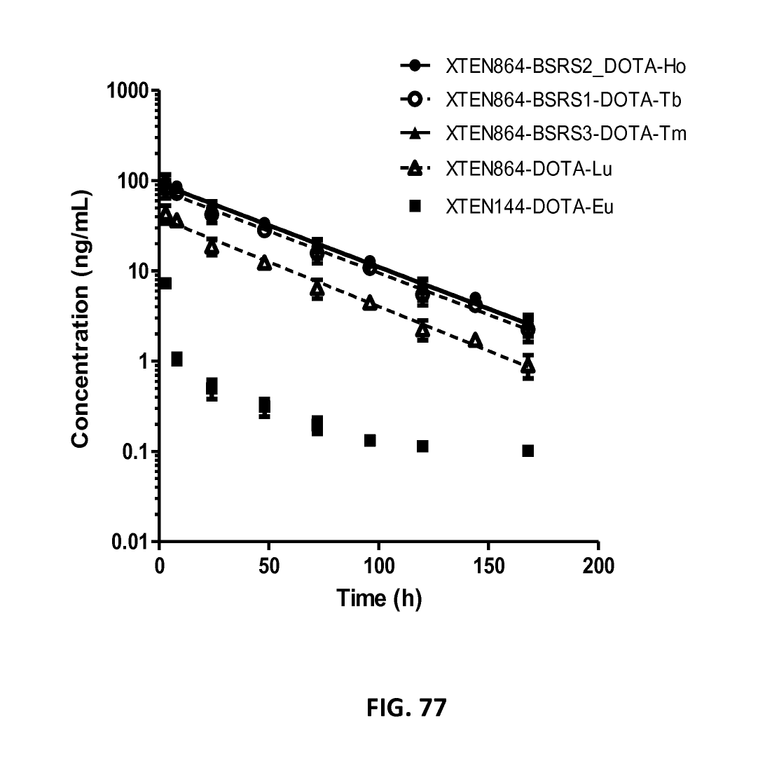

The T_{1/2} for XTEN864-BSRS1-DOTA-Tb was estimated to be 32.5 h; 32.6 h for XTEN864-BSRS2-DOTA-Ho; 32.5 h for XTEN864-BSRS3-DOTA-Tm and 30.6 h for XTEN864-DOTA-Lu (FIG. 77). In line with results observed in study 1, data in study 2 indicated that protease sites configured in tandem as represented by BSRS1, BSRS2 or BSRS-3 on XTEN also did not alter the T_{1/2} of the XTEN polymer. However, once cleaved, the molecule as exemplified by XTEN144-DOTA-Eu was rapidly cleared with an extremely short half-life of about 1.8 h.

### Example 40

### Binding Affinity of Protease-Treated and Untreated Anti-EpCAMscFv x Anti-CD3scFv-BSRS1-XTEN864 Bispecific Molecule

The PCM containing bispecific anti-EpCAMscFv x anti-CD3scFv-BSRS1-XTEN864 construct was used to evaluate the impact of protease treatment on binding affinity for the EpCAM ligand. Briefly, the recombinant anti-EpCAMscFv x anti-CD3scFv-BSRS1-XTEN864 molecule was treated or left untreated with MMP-9 for 2 h at 37° C. and analyzed in a dose range on an EpCAM/protein-L sandwich ELISA as follows: recombinant human EpCAM was coated onto a 96-well microtiter plate by an overnight incubation at 4° C. The wells were then blocked, washed and a dose range of protease treated anti-EpCAMscFv x anti-CD3scFv and untreated anti-EpCAMscFv x anti-CD3scFv-BSRS1-XTEN864 protein was added to the appropriate wells. After an h incubation to allow optimal capture of the protease-treated anti-EpCAMscFv x anti-CD3scFv and protease-untreated anti-EpCAMscFv x anti-CD3scFv-BSRS1-XTEN864 proteins by the coated EpCAM ligand, plate was washed again and peroxidase-conjugated protein L added. After an appropriate incubation period that allowed protein-L to bind to the kappa light of the scFvs, a final wash step was performed and tetramethylbenzidine (TMB) substrate added. TMB is a chromogenic substrate of peroxidase. Once desired color intensity was reached, 0.2 N sulfuric acid was introduced to stop the reaction and absorbance (OD) was measured at 450 nm using a spectrophotometer. The intensity of the color produced (i.e. OD) was plotted against the concentration of anti-EpCAMscFv x anti-CD3scFv and anti-EpCAMscFv x anti-CD3scFv-BSRS1-XTEN864 proteins. The concentration of analyte that gives half-maximal response (EC₅₀) was derived from the 4-parameter logistic regression equation.

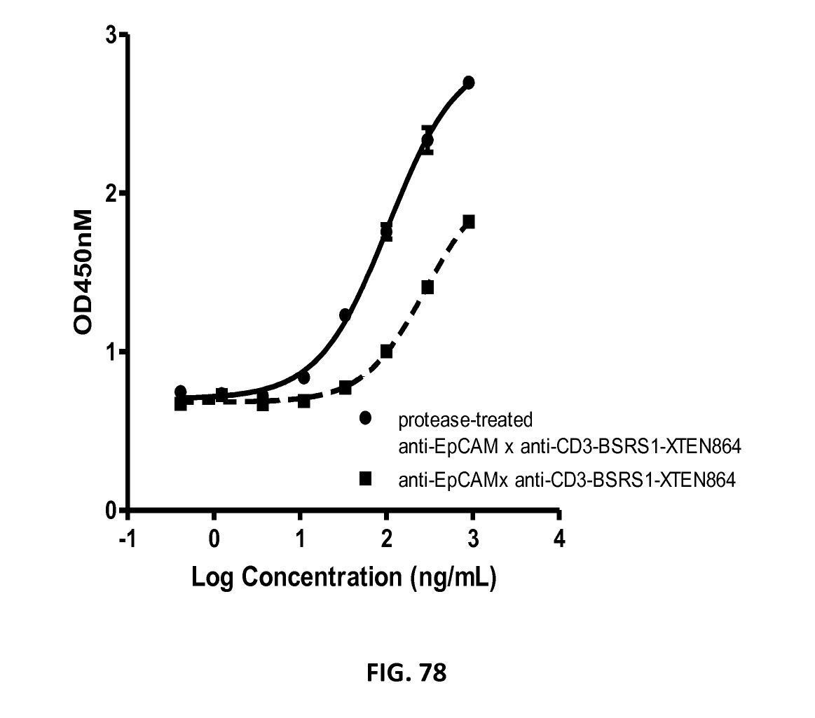

As shown in FIG. 78, the protease-treated anti-EpCAMscFv X anti-CD3scFv bispecific molecule has a stronger binding activity (EC₅₀ 106 nM) for the EpCAM ligand compared to the protease-untreated intact bispecific molecule (EC₅₀ 284 nM). Data suggest that the presence of XTEN864 hindered the binding of the anti-EpCAMscFv moiety for its ligand by at least 2.7 fold.

### Example 41

### In Vitro Selectivity Evaluation of Folate-XTEN Drug Conjugate

The ability of folate-XTEN drug conjugates to selectively target and kill cells bearing folate receptors was evaluated in vitro in a CellTiter-Glo anti-proliferation assay against a panel of folate receptor positive and negative cell lines selected from Table 39 and using FA-XTEN432-3xMMAF as a representative folate targeted XTEN drug conjugate.

TABLE 39
Folate receptor positive and negative cell lines

| Cell line | Tissue | Folate receptor status |
|---|---|---|
| KB | Nasopharyngeal | Positive |
| IGROV | Ovarian | Positive |
| SK-OV-3 | Ovarian | Positive |
| JEG-3 | Placenta | Positive |
| HeLa | Cervical | Positive |
| LoVo | Colorectal | Positive |
| SW620 | Colorectal | Positive |
| MDA-MB-231 | Breast | Positive |
| A549 | Lung | Negative |
| A375 | Multiple melanoma | Negative |
| LS-174T | Colorectal | Negative |
| SK-BR-3 | Breast | Negative |

In brief, selectivity evaluation of folate-XTEN432-3xMMAF was tested on folate-receptor positive KB (600 to 0.002 nM dose range, 4 fold serial dilution), JEG-3 (1000 to 0.02 nM dose range, 6 fold serial dilution) and SW620 (100 to 0.02 nM dose range, 4 fold serial dilution); and folate-receptor negative SK-BR-3 (100 to 0.02 nM, 4 fold serial dilution). As culture media contained high folic acid content, cells were grown and assay performed in folic acid free-RPMI supplemented with 10% heat-inactivated FCS at 37° C., 5% CO₂. Cells in log-phase were collected, counted and plated at 1×10⁴ cells per well onto a 96-well microtiter assay plate. Cells were allowed to adhere to the plate by an overnight incubation at 37° C., 5% CO₂. FA-XTEN432-3xMMAF in the presence and absence of folic acid were introduced in a dose range in duplicates and plate incubated for 3 days. Cells in experimental wells with folic acid inhibitor were pre-incubated with folic acid for 30 min at 37° C., 5% CO₂ before the addition of folate-XTEN432-3xMMAF. After the appropriate incubation period, CellTiter-Glo reagent was added to each well, mixed for 2 minute on an orbital shaker. Plate was then centrifuged at 90 g and incubated at room temperature for an additional 10 minutes to stabilize the luminescent signal Luminescence signals were then read on a luminometer & IC₅₀s (half maximal inhibitory concentration) calculated with GraphPad Prism or equivalent software. Quantitative IC₅₀s enabled FA-XTEN-3xMMAF cytotoxic selectivity against folate receptor positive versus negative cell lines to be compared.

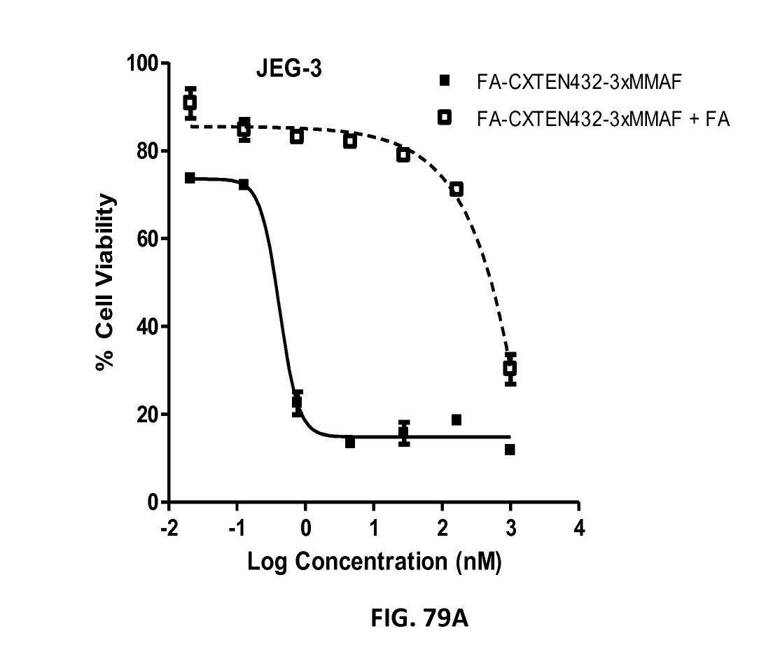

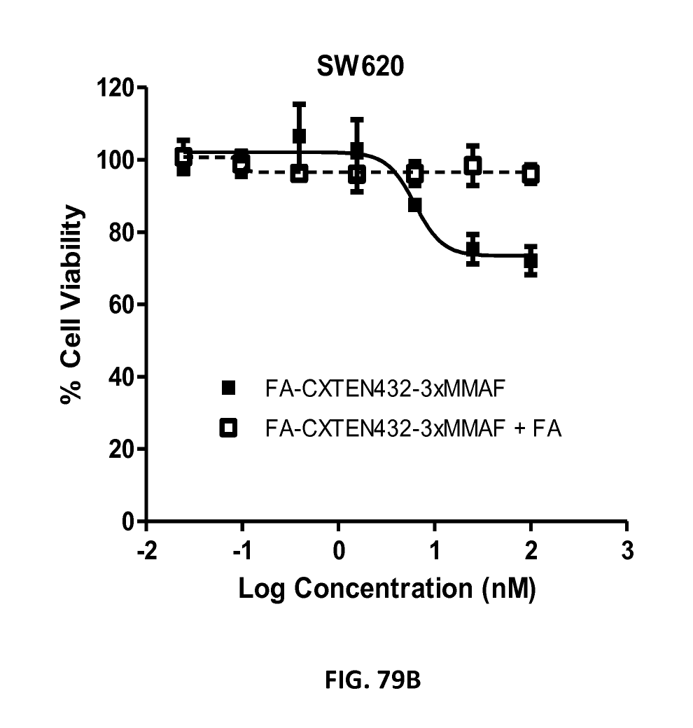

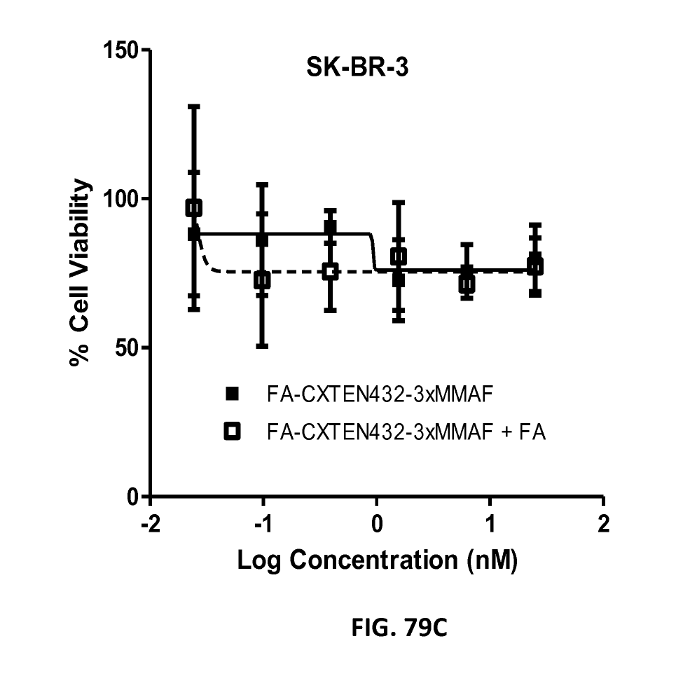

The FA-XTEN432-3xMMAF in the absence of folic acid inhibitor showed selective potent killing in folate receptor positive KB (IC₅₀ of 0.6 nM) (FIG. 58), JEG-3 (IC₅₀ of 0.4 nM) (FIG. 79A) and SW620 (IC₅₀ of 6.2 nM) (FIG. 79B) cells; but not on folate receptor negative SK-BR-3 (IC₅₀>100 nM) (FIG. 79C). Significantly, the cytotoxicity of FA-XTEN432-3xMMAF in KB, JEG-3 and SW620 was abrogated in the presence of folic acid indicating specificity of cytotoxic activity (FIG. 58, FIG. 79A and FIG. 79B).

### Example 42

### In Vivo Efficacy and Safety Evaluation of FA-3xMMAE-CCD-XTEN717 and FA-3xMMAE-CCD-BSRS1-XTEN713 Conjugates

To further tune the folate-XTEN drug conjugate for improved efficacy and safety as observed in example 36, the following modifications were introduced into the next generation folate conjugates: (1) MMAF toxin was replaced with MMAE; (2) MMAE were clustered at the N-terminal, close to the folate targeting moiety instead of evenly spaced on CXTEN; and (3) BSRS1 was placed immediately downstream of the MMAE toxin cluster. The two resultant proteins, FA-3xMMAE-CCD-XTEN717 and FA-3xMMAE-CCD-BSRS1-XTEN713 were evaluated in vivo in the KB xenograft model established in female nu/nu mice on a low-folate diet. In addition, instead of 3× injections for one week, only a single bolus injection was employed in this pilot study.

Seven days after KB cells implant, designated as Day 0, mice on low-folate diet bearing targeted tumor size of 100-200 mm³ were sorted into 2 groups of 3 animals per group. At Day 0, individual tumor volume of the mice enrolled in the study ranged from 83 to 147 mm³ and group mean tumor volume of the 2 groups were 111±33 and 111±30 mm³. Treatment was initiated on Day 0 with the intravenous administration of 120 nmol/kg equimolar concentration of FA-3xMMAE-CCD-XTEN717 and FA-3xMMAE-CCD-BSRS1-XTEN713. This is equivalent to administrating 9 mg/kg of folate-XTEN drug conjugate or 0.26 mg/kg of MMAE for both proteins (Table 40).

TABLE 40
Experimental design for dosing

| | | Total | Total | |
|---|---|---|---|---|
| | | conjugate | conjugate | |
| | | administered | administered | Total toxin |
| N | Agent | (mg/kg) | (nmol/kg) | equivalent |
| 8 | FA-CXTEN864- | 38 | 459 | 1 mg/kg |
| | 3xMMAF | | | MMAF |
| 3 | FA-3xMMAE-CCD- | 8.9 | 120 | 0.26 mg/kg |
| | XTEN717 | | | MMAE |
| 3 | FA-3xMMAE-CCD- | 9.0 | 120 | 0.26 mg/kg |
| | BSRS1-XTEN713 | | | MMAE |

Cessation or regression of tumor growth was determined through caliper measurement thrice per week until the study endpoint. The endpoint of the study was defined as tumor volume of 800 mm³ or 42 days, whichever comes first. To assess gross toxicity, animals were monitored individually for body weight three times per week. Any clinical signs of distress, deaths or adverse events were also recorded.

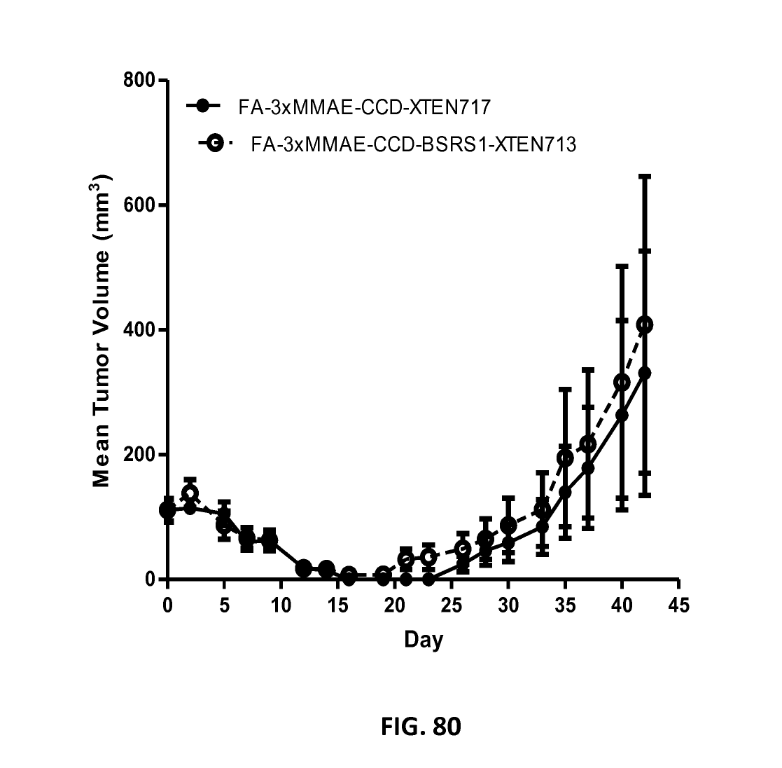

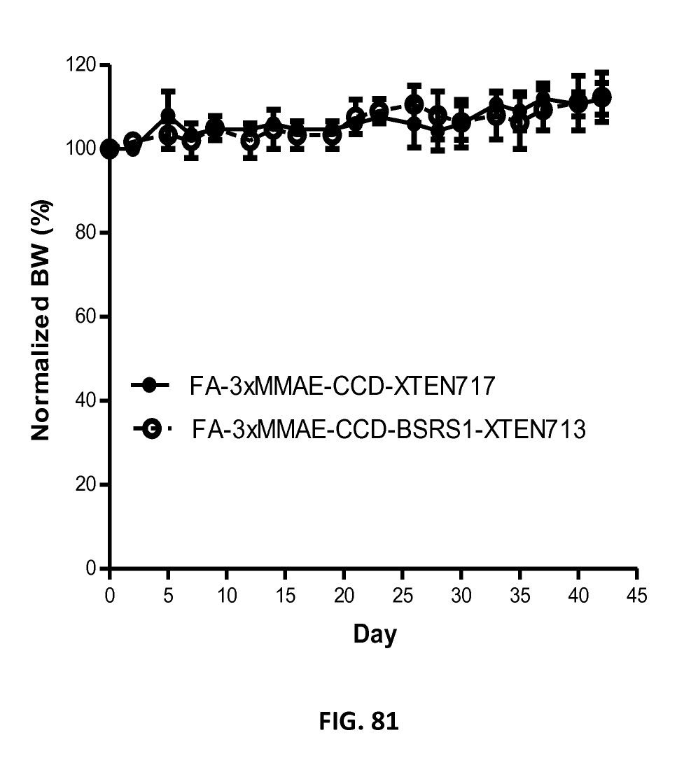

As shown in FIG. 80, both FA-3xMMAE-CCD-XTEN717 and FA-3xMMAE-CCD-BSRS1-XTEN713 drug conjugates were found to induce tumor regression for 19-21 days at a single bolus dose of 120 nmol/kg; after which tumor regrowth was observed for both conjugates. Although the number of mice per group is small, the tumor reduction efficacy of FA-3xMMAE-CCD-XTEN717 and FA-3xMMAE-CCD-BSRS1-XTEN713 appeared not to be significantly different from each other by student's t-test (p>0.05). Furthermore, FA-3xMMAE-CCD-XTEN717and FA-3xMMAE-CCD-BSRS1-XTEN713 were both found to be well tolerated with no visible weight loss (FIG. 81).

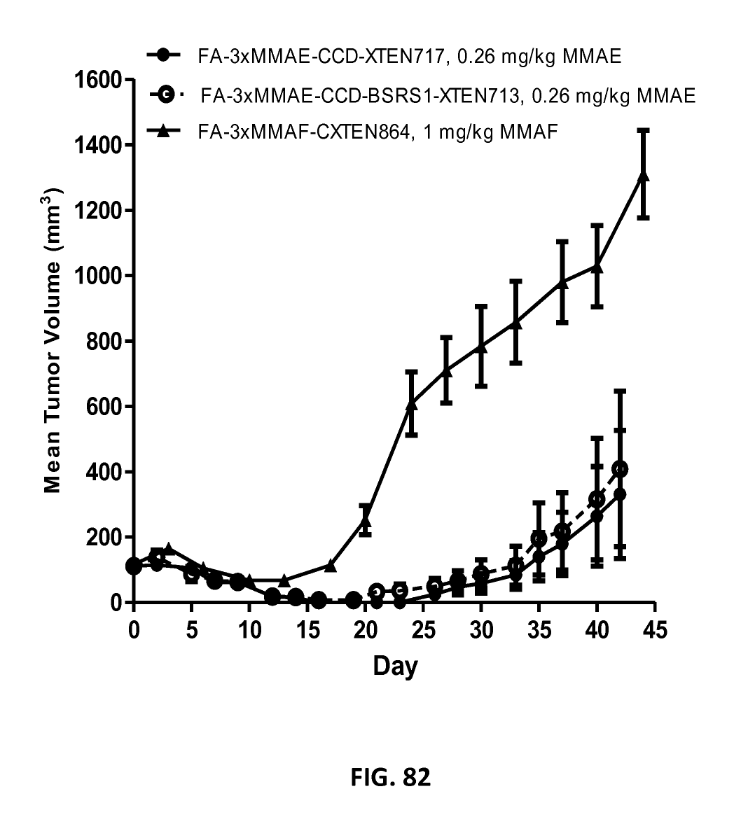

Significantly, as shown in FIG. 82 and reflected in Table 40, 120 nmol/kg (i.e. 9 mg/kg) of FA-3xMMAE-CCD-XTEN717and FA-3xMMAE-CCD-BSRS1-XTEN713 were drastically more effective than 459 nmol/kg (i.e. 38 mg/kg) of FA-XTEN864-3xMMAF in controlling tumor regression and growth.

Conclusions: The data strongly suggest that within the context of the KB xenograft model, FA-3xMMAE-CCD-XTEN717and FA-3xMMAE-CCD-BSRS1-XTEN713 are both highly efficacious and well tolerated, with efficacy achievable at 120 nmol/kg of drug conjugate. In this pilot KB xenograft study with limited number of animals, the BSRS1 composition appeared not to infer added advantage in tumor regression. Impressively, both FA-3xMMAE-CCD-XTEN717and FA-3xMMAE-CCD-BSRS1-XTEN713 were able to control tumor regression & growth at a substantially lower dose than that imparted by FA-XTEN864-3xMMAF. There are several factors that may account for this drastic improvement in efficacy; (1) MMAE is more effective than MMAF as the toxin payload; and/or (2) clustering of toxin at the N-terminal close to the targeting moiety may provide added advantage due to possible proteolytic cleavage of XTEN while in circulation.

### Example 43

### In Vivo Efficacy and Safety Evaluation of FA-3xMMAE-CCD-XTEN717, FA-3xMMAE-CXTEN864, FA-3xDM1-CCD-XTEN717, FA-3xMMAE-CCD-BSRS5-XTEN713 and FA-3xMMAE-CCD-BSRS6-XTEN13 Conjugates

To further understand and improve on the performance of the folate-XTEN drug conjugates evaluated in previous examples, the following studies will be executed to address the followings: (1) lowest dose concentration of FA-3xMMAE-CCD-XTEN717required to impart tumor stasis for 21 days; (2) impact of MMAE clustered at the N-terminal close to the folate targeting moiety versus MMAE evenly spaced on the CXTEN polymer; (3) will DM1 be an even better toxin payload than MMAE; (4) replace BSRS1 for BSRS5 and BSRS6 for enhance efficacy and safety margin; and (5) other in vivo folate receptor positive xenograft models.

Study 1: The FA-3xMMAE-CCD-XTEN717, FA-3xMMAE-CXTEN864 and FA-3xDM1-CCD-XTEN717 conjugates will be used to address questions 1, 2 and 3. Thirty-five female nu/nu mice on a low-folate diet with tumor volume in the range of 100 to 200 mm³ will be enrolled in the study as 7 groups of 5 mice per group. Designated as Day 0, treatment are initiated with the intravenous administration of FA-3xMMAE-CCD-XTEN717, FA-3xMMAE-CXTEN864 and FA-3xDM1-CCD-XTEN717 according to Table 41:

TABLE 41
Experimental design

| | | | Conjugate | Conjugate |
|---|---|---|---|---|
| Group | N | Protein | (nmol/kg) | (mg/kg) |
| 1 | 5 | Vehicle | 0 | 0 |
| 2 | 5 | FA-3xMMAE-CCD-XTEN717 | 120 | 8.9 |
| 3 | 5 | | 40 | 3.0 |
| 4 | 5 | | 13.3 | 1.0 |
| 5 | 5 | | 4.4 | 0.3 |
| 6 | 5 | FA-3xMMAE-CXTEN864 | 40 | 3.3 |
| 7 | 5 | FA-3xDM1-CCD-XTEN717 | 40 | 2.9 |

As the efficacy readout, tumor regression and growth are determined through caliper measurement thrice per week until the study endpoint. The endpoint of the study is defined as tumor volume of 800 mm³ or 30 days, whichever comes first. As an assessment of gross toxicity, animals are monitored individually for body weight three times per week. Any clinical signs of distress, deaths or adverse events are to be documented.

We anticipate the dose concentration of FA-3xMMAE-CCD-XTEN717capable of inducing tumor stasis for 21 days to fall within the dose range of 8.9 to 0.3 mg/kg. The head-to-head comparison of FA-3xMMAE-CCD-XTEN717versus FA-3xMMAE-CXTEN864 bearing the same nature and number of MMAE but differ in positioning will confirm if toxin positioning does indeed convey efficacy advantage. We speculate that toxin clustered at the N-terminal will be more efficacious or equivalent but certainly not inferior in efficacy to toxin configured to be evenly-spaced along the CXTEN polymer. In general, MMAE is believed to be more potent than DM1 in inducing cytotoxicity. We will evaluate toxin choice with the direct comparison of FA-3xMMAE-CCD-XTEN717against FA-3xDM1-CCD-XTEN717. Dosed at equimolar concentration, we predict FA-3xMMAE-CCD-XTEN717to be more effective than FA-3xDM1-CCD-XTEN717 in inducing tumor cessation and regression.

Study 2: The lack of performance of BSRS1 as described in Example 42 is likely influenced by the type and level of proteases present in the KB xenograft model used. To circumvent these possibilities, folate-XTEN drug conjugates bearing different BSRS composition will be tested in additional high folate-receptor expressing xenografts including but not limited to OVCAR3, IGROV3, OV-90, NCI-H2110 and LXFA-737. Specifically, FA-3xMMAE-CCD-BSRS1-XTEN713 will be evaluated in conjugation with FA-3xMMAE-CCD-BSRS5-XTEN713 and FA-3xMMAE-CCD-BSRS6-XTEN13 for their effectiveness in controlling tumor progression in KB, OVCAR and IGROV3 xenografts first. It is hypothesized that BSRS1, BSRS5 and BSRS6 PCM, each possessing different rate of protease susceptibility may behave differently in different tumor protease environment as represented by the different xenografts. When dosed at equimolar concentration, one BSRS may be more favorable than the others in the context of each xenograft.

### Example 44

### In Vitro Cytotoxicity and Specificity Evaluation of Anti-HER2scFv-3xMMAE-XTEN720, XTEN432-3xMMAE, anti-HER2scFv-3xDM1-XTEN720 and XTEN432-3xDM1 Conjugates

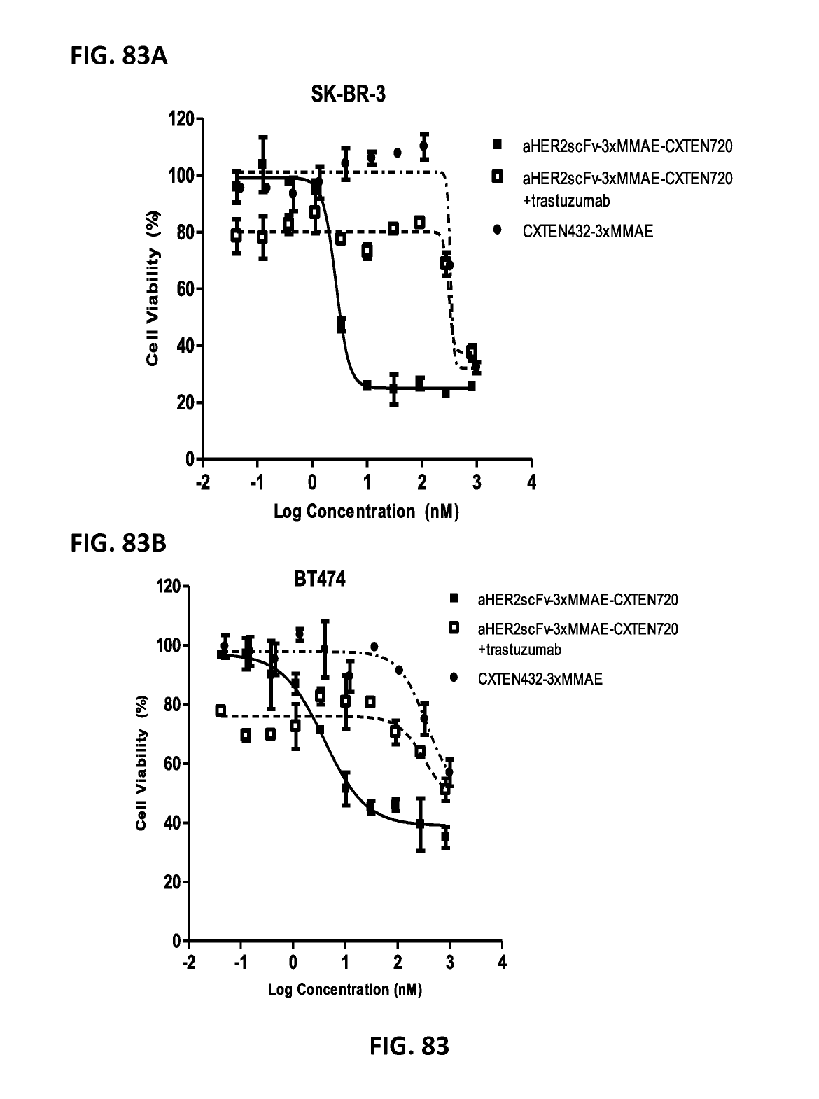

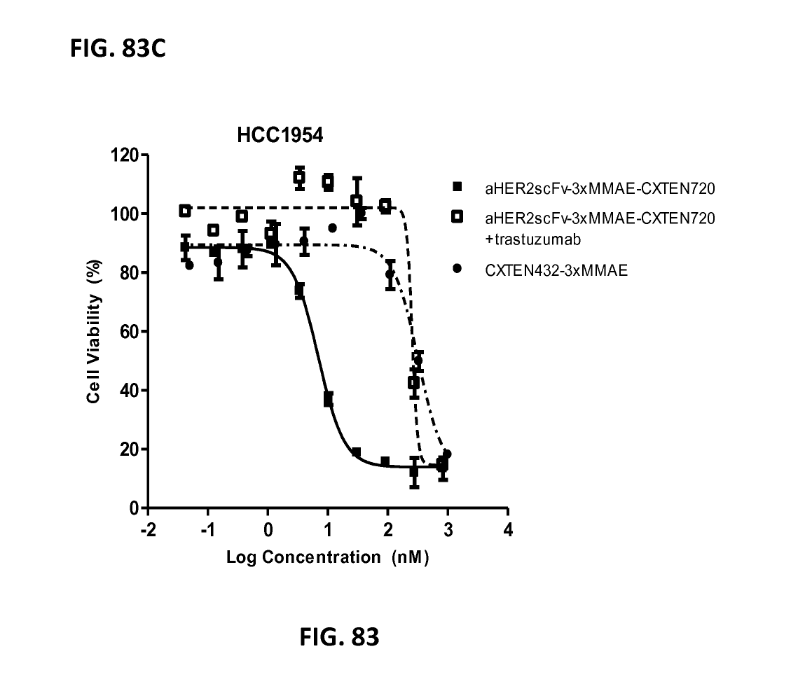

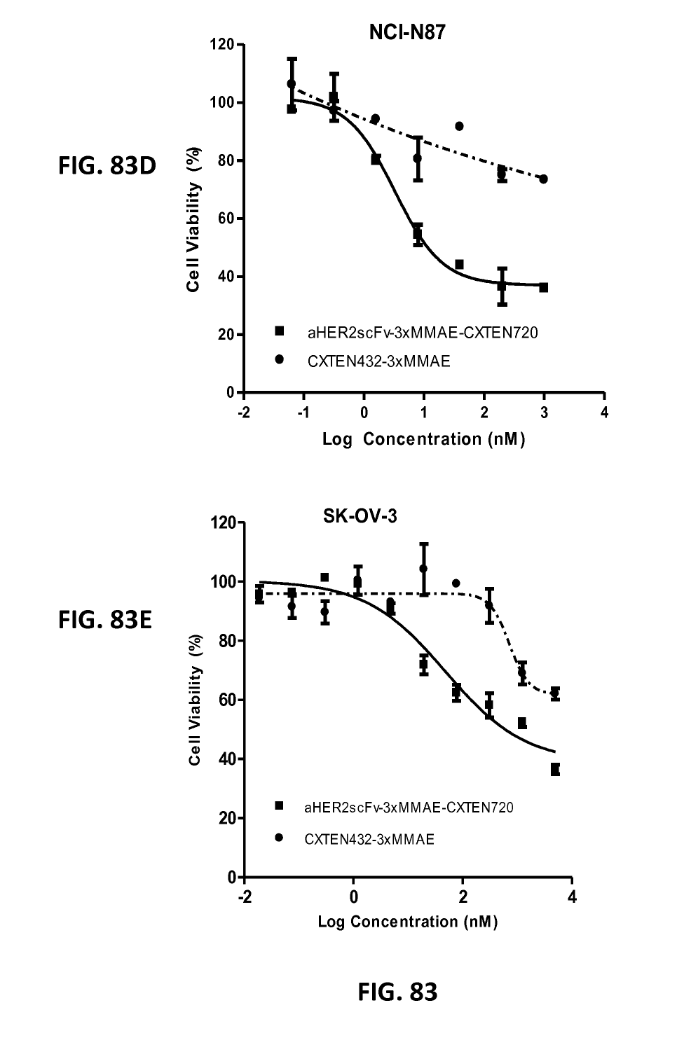

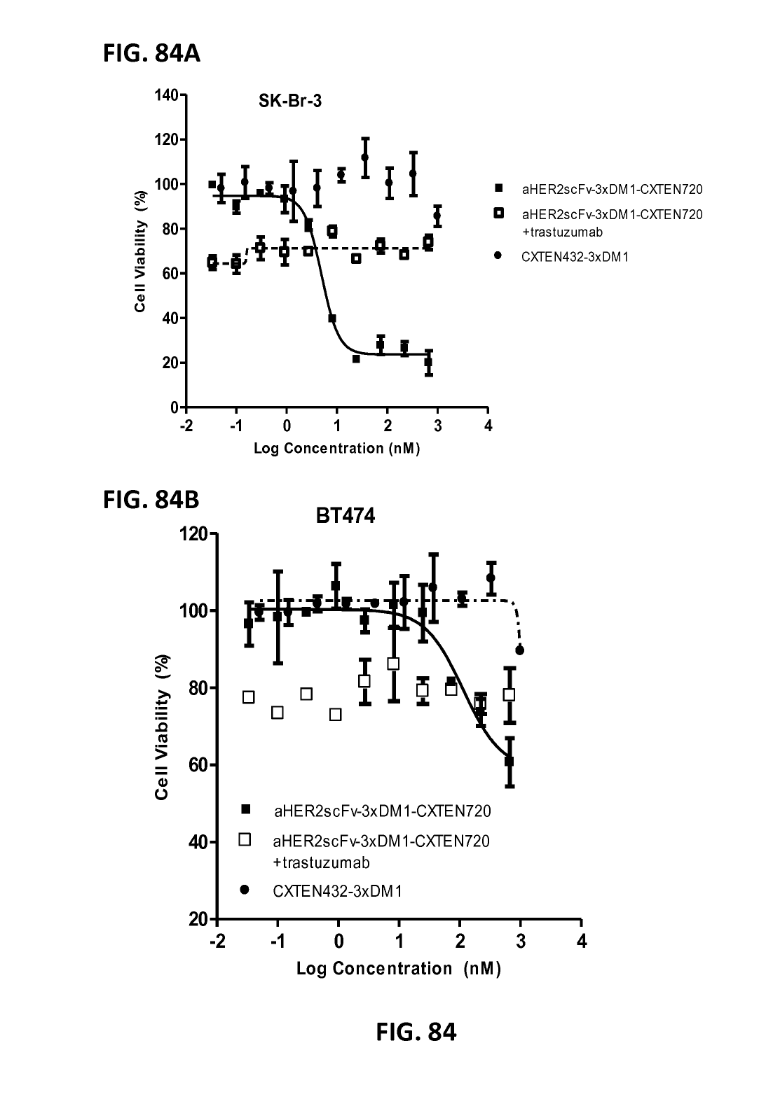

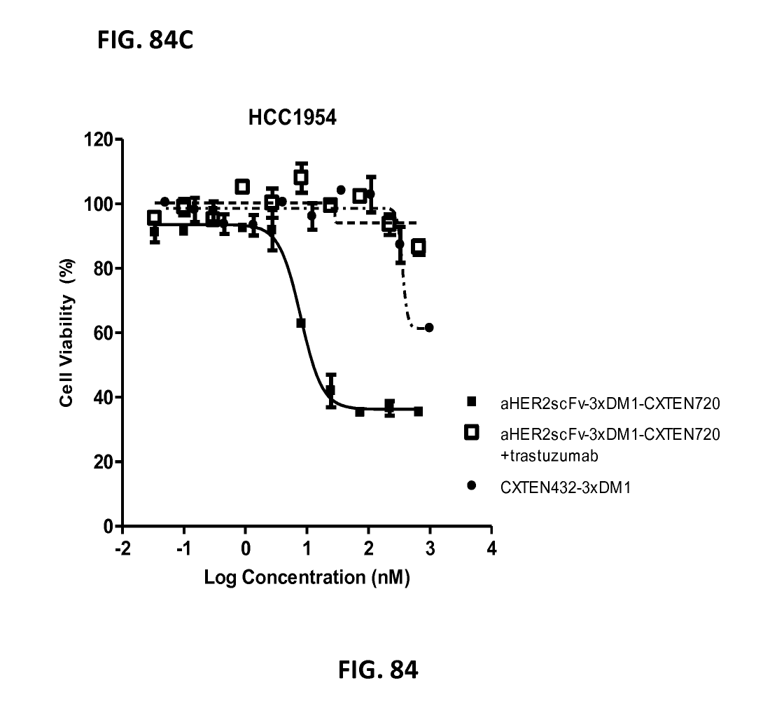

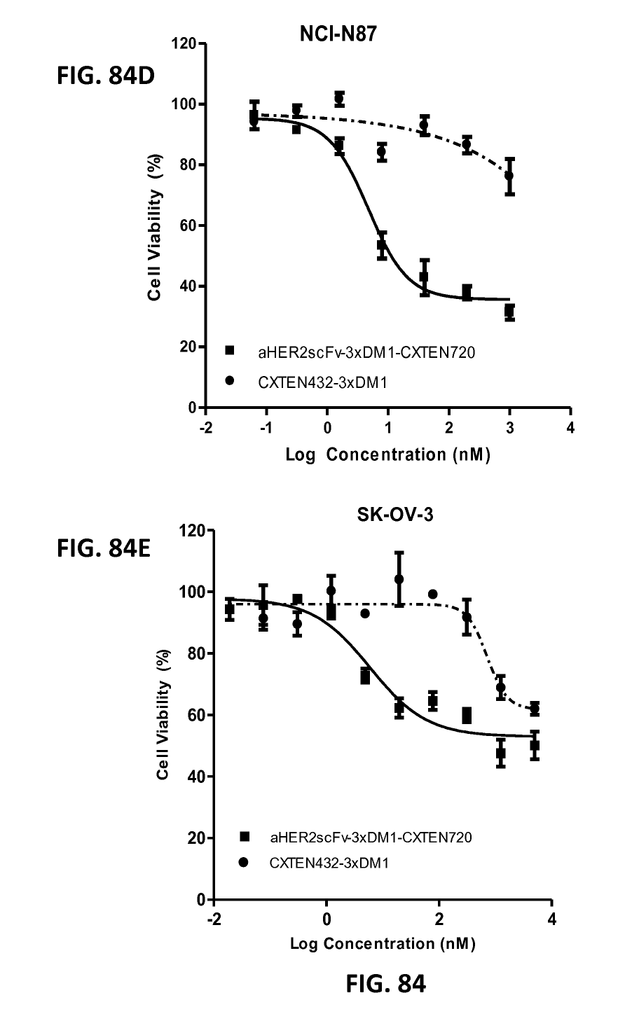

The specific cytotoxic activity of anti-HER2scFv-3xMMAE-XTEN720 and anti-HER2scFv-3xDM1-XTEN720 were compared to their corresponding non-targeting XTEN432-3xMMAE and XTEN432-3xDM1 precursors for their ability to effect cytotoxicity in a CellTiter-Glo cell viability assay. Using a panel of HER2 expressing cell lines which included but not limited to SK-BR-3, BT474, NCI-N87, SK-OV-3 and HCC1954, the specificity of anti-HER2scFv-3xMMAE-XTEN720 and anti-HER2scFv-3xDM1-XTEN720 were further elucidated in the presence and absence of trastuzumab as competitor (FIG. 83 and FIG. 84). Briefly, HER2 positive cells were plated at 1×10⁴ into each well of a 96-well microtiter plate and allowed to attach to the plate by an overnight incubation at 37° C., 5% CO₂. The anti-HER2scFv-3xMMAE-XTEN720 and anti-HER2scFv-3xDM1-XTEN720 conjugates, with and without trastuzumab treatment, as well as XTEN432-3xMMAE and XTEN432-3xDM1 conjugates were all introduced into the plate in a dose range (in duplicate) and plate was incubated for 3 days at 37° C., 5% CO₂. For SK-BR-3, BT474, HCC1954, a 3 fold serial dilution encompassing dose range of 1000 to 0.05 nM was employed; for NCI-N87, a 5 fold serial dilution of 1000 to 0.06 nM dose range; and SK-OV-3, a 4 fold serial dilution of 5000 to 0.19 nM dose range. After the appropriate incubation period, CellTiter-Glo reagent was added to each well, and the plate was mixed for 2 min on an orbital shaker. The plate was then centrifuged at 90×g and incubated at room temperature for an additional 10 min to stabilize the luminescent signal. Luminescence signals were then read on a luminometer and an IC₅₀s (half maximal inhibitory concentration) was calculated with GraphPad Prism software.

Results: As shown in FIG. 83 and Table 42, tested at equimolar concentration, anti-HER2scFv-3xMMAE-XTEN720, with the anti-HER2scFv targeting moiety, showed significantly higher cytotoxic killing than XTEN432-3xMMAE conjugate construct bearing no targeting moiety. Importantly, the addition of trastuzumab as competitor impaired the observed cell killing activity of anti-HER2scFv-3xMMAE-XTEN720 on the panel of cell lines tested, demonstrating the specificity of HER2-mediated cytotoxicity.

TABLE 42
IC50 determinations

| | IC₅₀ (nM) | | |
|---|---|---|---|
| | aHER2scFv- | aHER2scFv-3xMMAE- | |
| | 3xMMAE- | XTEN720 + | XTEN432- |
| Cell lines | XTEN720 | Trastuzumab | 3xMMAE |
| SK-BR-3 | 2.7 | >300 | >300 |
| BT474 | 4.0 | >300 | >300 |
| HCC1954 | 7.0 | >300 | >300 |
| NCI-N87 | 3.3 | NA | >1000 |
| SK-OV-3 | 48.7 | NA | >700 |

Similar results were obtained with the DM1-containing conjugates. The anti-HER2scFv-3xDM1-XTEN720 exhibited strong cytotoxicity that was inhibited by trastuzumab, while the corresponding non-targeted XTEN432-3xDM1 conjugate did not possess any significant in vitro cytotoxic activity (FIG. 84 and Table 43).

TABLE 43
IC50 determinations

| | IC₅₀ (nM) | | |
|---|---|---|---|
| | aHER2scFv- | aHER2scFv-3xDM1- | |
| | 3xDM1- | XTEN720 + | XTEN432- |
| Cell lines | XTEN720 | Trastuzumab | 3xDM1 |
| SK-BR-3 | 5 | >1000 | >1000 |
| BT474 | 113.7 | >1000 | >1000 |
| HCC1954 | 8 | >1000 | >360 |
| NCI-N87 | 4.8 | NA | >1000 |
| SK-OV-3 | 6 | NA | >700 |

Conclusions: The data suggest that under the experimental conditions, the anti-HER2scFv moiety imparts a HER2-mediated targeting effect that enhanced the cytotoxic activity of the XTEN-drug conjugates. Furthermore, both anti-HER2scFv-3xMMAE-XTEN720 and anti-HER2scFv-3xDM1-XTEN720 exerted strong cytotoxic activity in HER2 positive but trastuzumab resistant HCC1954 cell line; indicating XTEN-drug conjugates are more effective than trastuzumab antibody alone.

### Example 45

### In Vitro Cytotoxicity Evaluation of Anti-HER2scFv-3xMMAE-CCD-XTEN757 and anti-HER2scFv-3xMMAE-CCD-BSRS1-XTEN753 Conjugates

Using NCI-N87 cell line as a representative high HER2 expressing cell line, the cytotoxic activity of anti-HER2scFv-3xMMAE-CCD-XTEN757, anti-HER2scFv-3xMMAE-CCD-BSRS1-XTEN753 and protease-treated anti-HER2scFv-3xMMAE-CCD-BSRS1-XTEN753 were evaluated for their ability to effect cytotoxicity in a CellTiter-Glo cell viability assay. Briefly, NCI-N87 cells were plated at 1×10⁴ into each well of a 96-well microtiter plate and allowed to attach to the plate by an overnight incubation at 37° C., 5% CO₂. The anti-HER2scFv-3xMMAE-CCD-XTEN757, anti-HER2scFv-3xMMAE-CCD-BSRS1-XTEN753 and protease-treated anti-HER2scFv-3xMMAE-CCD-BSRS1-XTEN753 conjugates were all introduced into the plate in a 5 fold serial diluted 1000 to 0.06 nM dose range (in duplicate); and plate was incubated for 3 days at 37° C., 5% CO₂. After the appropriate incubation period, CellTiter-Glo reagent was added to each well, and the plate was mixed for 2 min on an orbital shaker. The plate was then centrifuged at 90×g and incubated at room temperature for an additional 10 min to stabilize the luminescent signal. Luminescence signals were then read on a luminometer and an IC₅₀s (half maximal inhibitory concentration) was calculated with GraphPad Prism software.

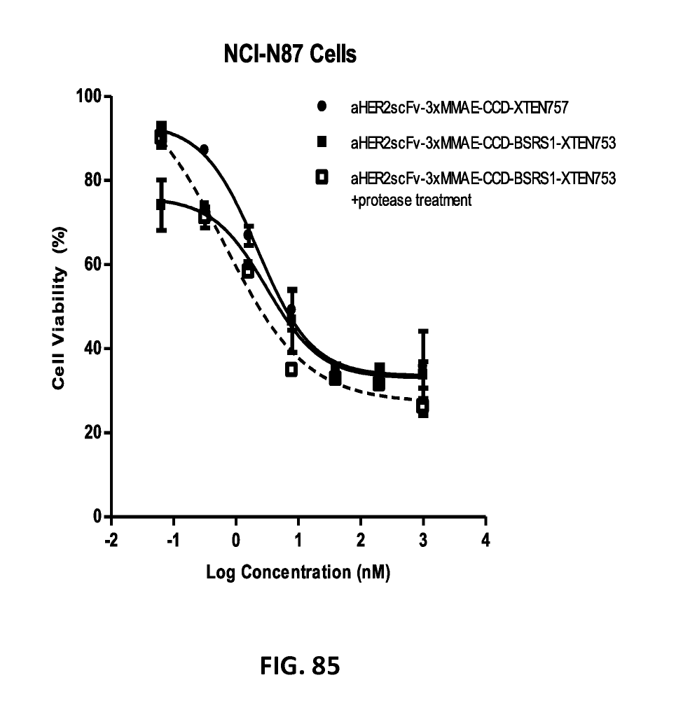

Results: As shown in FIG. 85, when tested at equimolar concentration, anti-HER2scFv-3xMMAE-CCD-XTEN757 and BSRS1 PCM containing intact anti-HER2scFv-3xMMAE-CCD-BSRS1-XTEN753 showed similar cytotoxic activity yielding an IC₅₀ of 2.2 nM and 3.1 nM respectively. However, upon protease treatment, the cleaved anti-HER2scFv-3xMMAE-CCD-BSRS1-XTEN753 exhibited a 5 fold stronger activity than the protease untreated anti-HER2scFv-3xMMAE-CCD-BSRS1-XTEN753 producing an IC₅₀ of 0.64 nM (FIG. 85).

Conclusions: The data suggest that under the experimental conditions, the protease-treated anti-HER2scFv-3xMMAE-CCD-BSRS1-XTEN753 imparts a stronger cytotoxic activity than its protease untreated counterpart.

### Example 46

### Efficacy and Safety Analysis of Anti-HER2scFv-3xMMAE-CXTEN720, Anti-HER2scFv-3xDM1-CXTEN720, anti-HER2scFv-3xMMAE-CCD-XTEN757, Anti-HER2scFv-3xMMAE-CCD-BSRS1-XTEN753 and Kadcyla in Female SCID Mice Bearing Human BT474 Breast Carcinoma

The following efficacy study is designed to decipher the effect of (1) MMAE versus DM1; (2) impact of MMAE clustered at the N-terminal close to the anti-HER2scFv targeting moiety versus MMAE evenly spaced on the CXTEN polymer; (3) impact of BSRS1; and performance of anti-HER2-XTEN drug conjugates in comparison to (4) benchmark Kadcyla.

Briefly, 1×10⁷ BT474 cells are injected subcutaneously in the flank of 68 female athymic nu/nu mice and allowed to form tumors, the size of which are measured with calipers and the volume calculated as 0.5×(L×W²), where L=length and W=width, in millimeters (mm) of the tumor. Designated as Day 0, 48 mice bearing targeted tumor size of 100-150 mm³ are selected from the 68 mice and sorted into 6 groups of 8 animals per group; with each group having approximately equivalent mean tumor volume. Treatment is initiated on Day 0 with the intravenous administration of PBS vehicle control, 30 nmol/kg of anti-HER2scFv-3xMMAE-CXTEN720, anti-HER2scFv-3xDM1-CXTEN720, anti-HER2scFv-3xMMAE-CCD-XTEN757, anti-HER2scFv-3xMMAE-CCD-BSRS1-XTEN753 and Kadcyla.

Cessation or regression of tumor growth is determined through caliper measurement three times per week till study endpoint. The endpoint of the study was defined as tumor volume of 1000 mm³ or 30 days, whichever comes first. Percent tumor growth inhibition (% TGI) is calculated for each treatment group and a % TGI 60% is considered therapeutically active. As an assessment of gross toxicity, animals are monitored individually for body weight three times per week till study endpoint. Any clinical signs of distress, deaths or adverse events are also noted.

It is expected that no tumor inhibition will be observed in BT474 tumor bearing mice administered with PBS vehicle. Based on the results obtained with the anti-HER2scFv-3xMMAE-XTEN720 drug conjugate in BT474 xenograft and performance of Kadcyla as documented in the literature, we expect all five drug conjugates (anti-HER2scFv-3xMMAE-CXTEN720, anti-HER2scFv-3xDM1-CXTEN720, anti-HER2scFv-3xMMAE-CCD-XTEN757, anti-HER2scFv-3xMMAE-CCD-BSRS1-XTEN753 and Kadcyla) to be more efficacious than vehicle in inducing tumor stasis or regression. (1) We speculate that MMAE will be more efficacious than DM1 and hence expect anti-HER2scFv-3xMMAE-CXTEN720 to out-perform anti-HER2scFv-3xDM1-CXTEN720 in tumor stasis and/or regression. (2) We believe anti-HER2scFv-3xMMAE-CCD-XTEN757 to be more effective or equivalent; but not inferior to anti-HER2scFv-3xMMAE-CXTEN720. (3) It remains to be seen if BT474 will provide the right tumor protease environment for BSRS1 PCM to be cleaved and thus for anti-HER2scFv-3xMMAE-CCD-BSRS1-XTEN753 to perform better than the corresponding anti-HER2scFv-3xMMAE-CCD-XTEN757 conjugate. (4) Dosed at equimolar, we anticipate one or more of the anti-HER2scFv-XTEN drug conjugate to out-perform Kadcyla due partly to scFv-XTEN having better tumor penetrability than a full length IgG. It is also expected that at 30 nmol/kg, all 5 drug conjugates compounds will be well tolerated with no body weight loss.

### Example 47

### Efficacy and Safety Analysis of Anti-HER2scFv-3xMMAE-CXTEN720, Anti-HER2scFv-3xDM1-CXTEN720, Anti-HER2scFv-3xMMAE-CCD-XTEN757, Anti-HER2scFv-3xMMAE-CCD-BSRS1-XTEN753 and Kadcyla in Female SCID Mice Bearing Human NCI-N87 Gastric Carcinoma and SK-OV-3 Ovarian Carcinoma

As a representation of tumor environment other than BT474, the anti-HER2scFv-3xMMAE-CXTEN720, anti-HER2scFv-3xDM1-CXTEN720, anti-HER2scFv-3xMMAE-CCD-XTEN757, anti-HER2scFv-3xMMAE-CCD-BSRS1-XTEN753 and Kadcyla will also be evaluated in NCI-N87 gastric carcinoma and SK-OV-3 ovarian carcinoma.

In the NCI-N87 xenograft model, 1x10⁷ NCI-N87 cells are subcutaneously administered into the flank of 68 female SCID mice; while in the SK-OV-3 model, 1 mm³ SK-OV-3 tumor fragment is subcutaneously implanted the flank of female athymic nu/nu mice. Designated as Day 0, 48 mice bearing targeted tumor size of 100-150 mm³ are selected from the 68 mice and sorted into 6 groups of 8 animals per group; with each group having approximately equivalent mean tumor volume. Treatment is initiated on Day 0 with the intravenous administration of PBS vehicle control, 30 nmol/kg of anti-HER2scFv-3xMMAE-CXTEN720, anti-HER2scFv-3xDM1-CXTEN720, anti-HER2scFv-3xMMAE-CCD-XTEN757, anti-HER2scFv-3xMMAE-CCD-BSRS1-XTEN753 and Kadcyla.

Cessation or regression of tumor growth is determined through caliper measurement three times per week till study endpoint. The endpoint of the NCI-N87 study is defined as tumor volume of 800 mm³ or 30 days, whichever comes first. The endpoint for the SK-OV-3 study is defined as 2000 mm³ or 30 days, whichever comes first. Percent tumor growth inhibition (% TGI) is calculated for each treatment group in both NCI-N87 and SK-OV-3 studies. Treatment group with % TGI 60% is considered therapeutically active. As an assessment of gross toxicity, animals are monitored individually for body weight three times per week till study endpoint. Any clinical signs of distress, deaths or adverse events are also noted.

It is expected that no tumor inhibition will be observed in NCI-N87 and SK-OV-3 tumor bearing mice administered with PBS vehicle. Based on the results obtained with the anti-HER2scFv-3xMMAE-XTEN720 drug conjugate in BT474 xenograft and performance of Kadcyla as documented in the literature in NCI-N87 and SK-OV-3 models, we expect all five drug conjugates (anti-HER2scFv-3xMMAE-CXTEN720, anti-HER2scFv-3xDM1-CXTEN720, anti-HER2scFv-3xMMAE-CCD-XTEN757, anti-HER2scFv-3xMMAE-CCD-BSRS1-XTEN753 and Kadcyla) to be more efficacious than vehicle in inducing tumor stasis or regression for both xenograft models. (1) As with the BT474 model, we speculate that MMAE will be more effective than DM1 and hence anticipate anti-HER2scFv-3xMMAE-CXTEN720 to perform better than anti-HER2scFv-3xDM1-CXTEN720 in inducing tumor cessation and/or regression. (2) We believe anti-HER2scFv-3xMMAE-CCD-XTEN757 to be more or of equivalent efficacy; but not inferior to anti-HER2scFv-3xMMAE-CXTEN720. (3) It remains to be seen either or both NCI-N87 and SK-OV-3 xenografts will provide the right tumor protease environment for BSRS1 PCM to be cleaved and thus for anti-HER2scFv-3xMMAE-CCD-BSRS1-XTEN753 to perform better than anti-HER2scFv-3xMMAE-CCD-XTEN757. (4) Dosed at equimolar, we anticipate one or more of the anti-HER2scFv-XTEN drug conjugate to out-perform Kadcyla partially due to scFv-XTEN having better tumor penetrability than a full length IgG. It is also expected that at 30 nmol/kg, all 5 drug conjugates compounds will be well tolerated with no body weight loss.

It is possible that BSRS1 PCM containing anti-HER2-XTEN drug conjugate may not provide added advantage in BT474, NCI-N87 or SK-OV-3 as compared to the corresponding non-BSRS1 containing conjugate. If this is indeed the case, the BSRS1 moiety will be replaced with other PCM and resulting conjugates will be evaluated in HER2 positive xenografts.

### Example 48

### Elastase Digestion and In Vitro Activity Test

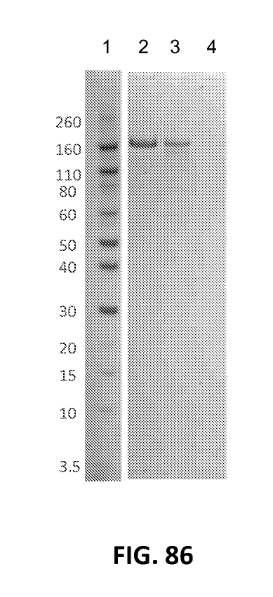

It had been speculated that neutrophil elastase, a serine protease with broad substrate specificity, may be able to degrade XTEN. An experiment was designed to demonstrate the susceptibility of XTEN to protease cleavage. 10 μM of purified XTEN_AE864 was incubated with 1:1000 or 1:100 molar ratio (elastase:XTEN) of human neutrophil elastase (R&D Systems) at 37° C. for 2 hours. Samples before and after digestion were analyzed on SDS-PAGE gel followed with coomassie staining (FIG. 86). Partial degradation was observed for XTEN_AE864 when incubating with neutrophil elastase at the 1:1000 molar ratio, and XTEN_AE864 was completely digested by neutrophil elastase at the 1:100 molar ratio.

In the context of targeted conjugate compositions wherein the cytotoxic drugs are conjugated to the CCD, the compositions are designed so that the cytotoxic drugs are in closer proximity with the targeting moiety compared with targeted compositions wherein the payloads are conjugated to regular cysteine containing XTEN and the cysteine are dispersed across the length of the XTEN. The rationale for the design was that when subjected to protease digestion on XTEN, a higher percentage of drug molecules linked to the CCD in the targeted conjugate compositions are more likely to stay linked with the associated targeting moiety compared to drugs linked more distally in the XTEN, which will lead to higher probability of the drug molecules getting internalized when the targeting moiety binds the target tumor cell, leading to cell death. Since the in vivo environment has a lot of various proteases that may degrade XTEN, the targeted conjugate compositions with payloads linked to the CCD would therefore result in better in vivo efficacy than regular cysteine-containing XTEN. Methods described below can be used to test this hypothesis in vitro.

Folate-CCD-XTEN-3xDM1 (containing 3 molecules of DM1, as representative of a targeted conjugate composition) and folate-CXTEN-3xDM1 (containing 3 molecules of DM1, as representative of a targeted XTEN with the DM1 payloads conjugated to the XTEN) prepared with similar methods as described in Example 13 and Example 20 will be incubated with neutrophil elastase at 1:1000 molar ratio at 37° C. Samples will be removed at different time points and will be monitored by SDS-PAGE to monitor the degradation progress, and will be evaluated in a cell-based assay for their killing activity in KB cells, which have folate receptors, as described in Example 35. We expect that at certain time point, the samples will reach a degradation level that a low percentage of the folate-CXTEN-3xDM1 will still retain all 3 of the DM1 drugs linked with folate, while a significantly higher percentage of the folate-CCD-XTEN-3xDM1 will retain all 3 DM1 drugs linked with folate. Because DM1 linked to XTEN without folate is not toxic to cells, the cytotoxic effect is only contributed by DM1 that remain linked to the folate targeting moiety. When testing these samples in the cell-based assay, we expect similar IC50 for all samples since this is only relevant for the affinity between folate and its receptor, but the percentage of cell killing would be expected to become lower as the DM1 drug falls off from the folate as a result of proteolysis of the composition.

### Example 49

### In Vitro Cytotoxicity Evaluation of FA-CCD1-3xMMAE-AE717, FA-CCD1-3xMMAE-BSRS1-AE713, FA-CCD7-3xMMAE-BSRS4-AE717 and the Corresponding Non-Targeted Conjugates

The cytotoxic activity of FA-CCD1-3xMMAE-AE717, protease-treated and untreated FA-CCD1-3xMMAE-BSRS1-AE713, protease-treated and untreated FA-CCD7-3xMMAE-BSRS4-AE717 and the corresponding non-targeted controls CCD1-3xMMAE-AE717, CCD1-3xMMAE-BSRS1-AE713 and CCD7-3xMMAE-BSRS4-AE717 were compared for the ability to effect cytotoxicity in an in vitro assay utilizing the folate receptor positive KB cell line. The KB cells were grown and the assay performed in folic acid-free RPMI plus 10% heat-inactivated fetal calf serum to minimize folic acid content in the cell culture. Briefly, KB cells were plated at 1×10⁴ into each well of a 96-well microtiter plate and allowed to attach to the plate by an overnight incubation at 37° C., 5% CO₂. All eight proteins were all introduced into the plate as a 4 fold serial dilution dose range with initial concentration ranging from 192 to 81 nM. After 72 h incubation, CellTiter-Glo reagent was added to each well according to manufacturer's instruction and luminescence signals read on a luminometer; and IC₅₀s calculated with GraphPad Prism software.

Results: As shown in Table 44, FA-CCD1-3xMMAE-AE717, FA-CCD1-3xMMAE-BSRS1-AE713 and FA-CCD7-3xMMAE-BSRS4-AE717 all showed similar and strong cytotoxic activity with IC₅₀ of 1.41±0.1 nM, 1.55 nM and 1.05 nM respectively. Data demonstrated that the introduction of intact CCD and BSRS sequences have no apparent impact on in vitro cytotoxic activity in KB cells. As expected, all the non-targeted CCD1-3xMMAE-AE717, protease-cleaved non-targeted CCD1-3xMMAE-BSRS1-AE713 and protease-cleaved non-targeted CCD7-3xMMAE-BSRS4-AE717 exhibited minimal cytotoxicity (IC₅₀>100 nM). Interestingly, protease-treated and untreated FA-CCD1-3xMMAE-BSRS1-AE713 yielded equivalent activity (IC₅₀ 1.5 nM versus 1.1±1.7 nM). Similarly, protease-treated and untreated FA-CCD7-3xMMAE-BSRS4-AE717 also exhibited comparable cytotoxicity ((IC₅₀ 1.05 nM versus 2.4±0.9 nM).

TABLE 44
IC50 determinations

| | Pro- | | | | |
|---|---|---|---|---|---|
| | tease- | IC50 (nM) | | | |
| | treat- | Assay | Assay | Aver- | |
| Conjugate | ment | 1 | 2 | age | SD |
| FA-CCD1-3xMMAE-AE717 | No | 1.32 | 1.5 | 1.14 | 0.1 |
| FA-CCD1-3xMMAE-BSRS1- | No | | 1.55 | | |
| AE713 | | | | | |
| FA-CCD7-3xMMAE-BSRS4- | No | | 1.05 | | |
| AE717 | | | | | |
| FA-CCD1-3xMMAE-BSRS1- | Yes | 1.1 | 1.17 | 1.1 | 1.7 |
| AE713 | | | | | |
| FA-CCD7-3xMMAE-BSRS4- | Yes | 1.75 | 3.01 | 2.4 | 0.9 |
| AE717 | | | | | |
| CCD1-3xMMAE-AE717 | No | >100 | >100 | | |
| CCD1-3xMMAE-BSRS1-AE713 | Yes | >100 | | | |
| CCD7-3xMMAE-BSRS4-AE717 | Yes | >100 | | | |

Conclusions: The data suggest that under the experimental conditions described, XTEN did not hinder the folate targeting moiety from its interaction with the folate receptors present on the KB cells.

### Example 50

### Pharmacokinetics and Bio-Distribution Analysis of BSRS-XTEN864 in the 11292 Xenograft Model

The NCI-H292 xenograft has been reported in the literature to possess the right tumor environment to successfully cleave RS3. All BSRS PCM contains the RS3 sequence. To better evaluate and screen for efficacy of the different BSRS PCM for in vivo cleavage, we will utilize the NCI-H2892 xenograft model to evaluate BSRS1-XTEN864, BSRS2-XTEN864, BSRS3-XTEN864, BSRS4-XTEN864, BSRS5-XTEN864 and BSRS6-XTEN864 via a bio-distribution study. Each BSRS bearing XTEN864 and the non-BSRS containing XTEN864 control will be orthogonally labeled with 2 rare lanthanide earth ions, one metal at the N-terminal and the other metal immediately downstream of the BSRS sequence. Using 6 available rare earth metals, 3 conjugates are evaluated per group as a mixture.

Briefly, six groups of 3 mice per group of female SCID mice bearing NCI-H292 tumor volume of 250-350 mm³ will be injected with a mixture of 3 conjugates (two dual-labeled BSRS-XTEN864 and one dual-labeled XTEN864 control) per group. For tissue bio-distribution analysis, 3 mice are sacrificed on day 5. At terminal endpoint, tumor, heart, kidneys, liver, lungs, spleen, pancreas and brain are harvested, rinsed in PBS, blotted dry, weigh and snap frozen. The amount of the respective metal present in the various tissues is quantitated by ICP-MS and data express as percent injected dose per g tissue (% ID/g).

For the BSRS that is cleaved in the NCI-H292 tumor environment, on day 5, only 1 metal (conjugated immediately downstream of the BSRS sequence) will be detected in the tumor sample; the N-terminal positioned metal of this BRSR-XTEN864 will be eliminated by day 5 due to its short half-life of a few hours. For BSRS that is not cleaved by H292 tumor environment, both metals (i.e. N-terminal positioned and downstream of the BSRS sequence) will be detected. In this manner, an in vivo functional BSRS could be identified for use in targeted-XTEN-drug conjugates.

### Example 51

### Analytical Size Exclusion Chromatography of XTEN Linked with Diverse Payloads

Size exclusion chromatography analyses were performed on fusion proteins containing various therapeutic proteins and unstructured recombinant proteins of increasing length. An exemplary assay used a TSKGel-G4000 SWXL (78 mm×30cm) column in which 40 μg of purified glucagon fusion protein at a concentration of 1 mg/ml was separated at a flow rate of 0.6 ml/min in 20 mM phosphate pH 6.8, 114 mM NaCl. Chromatogram profiles were monitored using OD214 nm and OD280 nm. Column calibration for all assays were performed using a size exclusion calibration standard from BioRad; the markers include thyroglobulin (670 kDa), bovine gamma-globulin (158 kDa), chicken ovalbumin (44 kDa), equine myoglobuin (17 kDa) and vitamin B12 (1.35 kDa). Based on the SEC analyses for all constructs evaluated, the apparent molecular weights, the apparent molecular weight factor (expressed as the ratio of apparent molecular weight to the calculated molecular weight) and the hydrodynamic radius (R_{H} in nm) are shown in Table 45. The results indicate that incorporation of different XTENs of 576 amino acids or greater confers an apparent molecular weight for the fusion protein of approximately 339 kDa to 760, and that XTEN of 864 amino acids or greater confers an apparent molecular weight greater than approximately 800 kDA. The results of proportional increases in apparent molecular weight to actual molecular weight were consistent for fusion proteins created with XTEN from several different motif families; i.e., AD, AE, AF, AG, and AM, with increases of at least four-fold and ratios as high as about 17-fold. Additionally, the incorporation of XTEN fusion partners with 576 amino acids or more into fusion proteins with the various payloads (and 288 residues in the case of glucagon fused to Y288) resulted with a hydrodynamic radius of 7 nm or greater; well beyond the glomerular pore size of approximately 3-5 nm. The addition of 3 different lengths of XTEN to the scFv of anti-Her2 resulted in increases in Apparent Molecular Weight and hydrodynamic radius that were proportional to the increase in XTEN length, indicating that the properties of scFv can be adjusted depending on the desired properties, but that with an XTEN as short as 288 amino acids, the hydrodynamic radius is larger than the renal pore size. Accordingly, it is expected that fusion proteins comprising XTEN have reduced renal clearance, contributing to increased terminal half-life and improving the therapeutic or biologic effect relative to a corresponding un-fused biologic protein or antibody fragment.

TABLE 45
SEC analysis of various polypeptides

| | | | | | Apparent | |
|---|---|---|---|---|---|---|
| Con- | XTEN or | | Actual | Appar- | Molecular | |
| struct | fusion | Therapeutic | MW | ent MW | Weight | R_{H} |
| Name | partner | Protein | (kDa) | (kDa) | Factor | (nm) |
| AC14 | Y288 | Glucagon | 28.7 | 370 | 12.9 | 7.0 |
| AC28 | Y144 | Glucagon | 16.1 | 117 | 7.3 | 5.0 |
| AC34 | Y72 | Glucagon | 9.9 | 58.6 | 5.9 | 3.8 |
| AC33 | Y36 | Glucagon | 6.8 | 29.4 | 4.3 | 2.6 |
| AC89 | AF120 | Glucagon | 14.1 | 76.4 | 5.4 | 4.3 |
| AC88 | AF108 | Glucagon | 13.1 | 61.2 | 4.7 | 3.9 |
| AC73 | AF144 | Glucagon | 16.3 | 95.2 | 5.8 | 4.7 |
| AC53 | AG576 | GFP | 74.9 | 339 | 4.5 | 7.0 |
| AC39 | AD576 | GFP | 76.4 | 546 | 7.1 | 7.7 |
| AC41 | AE576 | GFP | 80.4 | 760 | 9.5 | 8.3 |
| AC52 | AF576 | GFP | 78.3 | 526 | 6.7 | 7.6 |
| AC398 | AE288 | FVII | 76.3 | 650 | 8.5 | 8.2 |
| AC404 | AE864 | FVII | 129 | 1900 | 14.7 | 10.1 |
| AC85 | AE864 | Exendin-4 | 83.6 | 938 | 11.2 | 8.9 |
| AC114 | AM875 | Exendin-4 | 82.4 | 1344 | 16.3 | 9.4 |
| AC143 | AM875 | hGH | 100.6 | 846 | 8.4 | 8.7 |
| AC302 | AE912 + | hGH | 119.1 | 2,287 | 19.2 | 11.0 |
| | AE144 | | | | | |
| AC227 | AM875 | IL-1ra | 95.4 | 1103 | 11.6 | 9.2 |
| AC228 | AM1318 | IL-1ra | 134.8 | 2286 | 17.0 | 10.5 |
| AC493 | AE864 | Factor IX | 127.7* | 3967 | 31.1 | 12.2 |
| AC616 | AE864 | GLP2-2G | 83.1 | 1427 | 17.2 | 10 |
| AC647 | AE864 | Ghrelin | 82.7 | 996 | 12 | 9.2 |
| AC659 | AE864 | C-peptide | 82.7 | 822 | 10 | 8.8 |
| AC663 | AE1296 | C-peptide | 122.2 | 2348 | 19.2 | 11.1 |
| AC434 | AE288 | aaT | 71.1 | 500 | 7.0 | 7.7 |
| AC435 | AE576 | aaT | 97.5 | 1,127 | 11.6 | 9.5 |
| AC345 | AM875 | aaT | 122.6 | 1,390 | 11.3 | 9.9 |
| AC450 | AE288 | aHer2_scFv | 56.2 | 312 | 5.5 | 6.7 |
| AC451 | AE576 | aHer2_scFv | 82.6 | 760 | 9.2 | 8.6 |
| AC452 | AE864 | aHer2_scFv | 109.1 | 1,390 | 12.7 | 9.9 |

*excluding glycosylation

1.-105. (canceled)

106. A cysteine containing domain (CCD) comprising at least 12 to about 144 amino acid residues, between 3 and 9 of which are cysteine residues, wherein the CCD is characterized in that:
(a) at least 90% of the non-cysteine residues consist of 3 to 6 types of amino acids selected from the group consisting of glycine (G), alanine (A), serine (S), threonine (T), glutamate (E) and proline (P);
(b) no three contiguous amino acids are identical unless the amino acid is cysteine or serine; and
(c) no glutamate residue is adjacent to a cysteine residue.

107. The CCD of claim 106, wherein at least one cysteine residue is located within 9 consecutive amino acid residues counting from an N-terminus or a C-terminus of the CCD.

108. The CCD of claim 106, wherein the CCD sequence has at least 90% sequence identity to a sequence set forth in Table 6.

109. A recombinant polypeptide comprising the CCD of claim 106 and an extended recombinant polypeptide (XTEN), said XTEN comprising at least 144 to about 864 amino acids devoid of cysteine residues, and wherein said XTEN consists of at least 3 types of amino acids selected from the group consisting of glycine (G), alanine (A), serine (S), threonine (T), glutamate (E), and proline (P).

110. The recombinant polypeptide of claim 109, wherein the XTEN 15 characterized in that:
(a) it has a molecular weight that is at least 4-fold greater than the molecular weight of the CCD; and
(b) it has at least 90% sequence identity to a sequence set forth in Table 10.

111. The recombinant polypeptide of claim 109, further comprising a peptidic cleavage moiety (PCM) between the CCD and the XTEN, wherein the PCM is capable of being cleaved by a mammalian protease.

112. The recombinant polypeptide of claim 111, wherein the PCM is capable of being cleaved by a mammalian protease set forth in Table 7.

113. The recombinant polypeptide of claim 111, further comprising a payload molecule chemically conjugated to a thiol group of a cysteine residue of the CCD by a cross-linker.

114. The recombinant polypeptide of claim 113, wherein the payload molecule is a cytotoxic drug selected from the group consisting of doxorubicin, nemorubicin, PNU-159682, paclitaxel, docetaxel, auristatin E, auristatin F, dolastatin 10, dolastatin 15, monomethyl auristatin E (MMAE), monomethyl auristatin F (MMAF), monomethyl auristatin D (MMAD), maytansine, mertansine (DM1), maytansinoid DM4, calicheamicin, N-acetyl-calicheamicin, vinblastine, vincristine, vindesine, vinorelbine, camptothecin, topotecan, irinotecan, SN-38, duocarmycin A, duocarmycin B1, duocarmycin B2, duocarmycin C1, duocarmycin C2, duocarmycin D, duocarmycin SA, duocarmycin TM, duocarmycin MB, duocarmycin DM, mitomycin C, rachelmycin, epothilone A, epothilone B, epothilone C, tubulysin B, tubulysin M, pyrrolobenzodiazepine (PBD), and bortezomib.

115. The recombinant polypeptide of claim 114, wherein the payload molecule is monomethyl auristatin E (MMAE) or monomethyl auristatin F (MMAF).

116. The recombinant polypeptide of claim 114, wherein the payload molecule is mertansine (DM1).

117. The recombinant polypeptide of claim 113, wherein the payload molecule is a biologically active protein selected from the group consisting of TNFα, IL-12, ranpirnase, human ribonuclease (RNAse), bovine pancreatic RNase, pokeweed antiviral protein, Pseudomonas exotoxin A, gelonin, ricin-A, interferon-alpha, interferon-lambda, urease, amatoxin, alpha-amanitin, beta-amanitin, gamma-amanitin, epsilon-amanitin, bouganin, and staphylococcal enterotoxin.

118. The recombinant polypeptide of claim 113, wherein the recombinant polypeptide is fully conjugated such that a payload molecule is conjugated to each cysteine residue of the CCD.

119. The recombinant polypeptide of claim 118, wherein the recombinant polypeptide is capable of being separated from a recombinant polypeptide that is not fully conjugated with a peak separation value >6, wherein the peak separation value is defined as (t_{R2}-t_{R1})/FWHM, wherein
(a) t_{R2} represents retention time by reverse phase HPLC of a peak corresponding to the recombinant polypeptide that is fully conjugated;
(b) t_{R1} represents retention time by reverse phase HPLC of a peak corresponding to the recombinant polypeptide that is not fully conjugated and that is closest to the peak corresponding to the fully conjugated recombinant polypeptide; and
(c) FWHM represents full width at half maximum of the peak corresponding to the recombinant polypeptide that is fully conjugated.

120. The recombinant polypeptide of claim 113, further comprising a first targeting moiety (TM), wherein the first TM is selected from the group consisting of an IgG antibody, a Fab fragment, a F(ab′)2 fragment, a scFv, a scFab, a dAb, a single domain heavy chain antibody, and a single domain light chain antibody, and wherein the TM is capable of specifically binding a ligand associated with a target tissue.

121. The recombinant polypeptide of claim 120, wherein the first TM is an IgG antibody.

122. The recombinant polypeptide of claim 120, wherein the first TM is linked to an N-terminus or a C-terminus of the CCD.

123. The recombinant polypeptide of claim 120, wherein the ligand is associated with a tumor, a cancer cell, or a tissue with an inflammatory condition.

124. A pharmaceutical composition, comprising the recombinant polypeptide of claim 120 and a pharmaceutically acceptable carrier.

125. The pharmaceutical composition of claim 124 for use in the treatment of a disease in a subject, wherein the disease is selected from the group consisting of breast cancer, ER/PR+ breast cancer, Her2+ breast cancer, triple-negative breast cancer, liver carcinoma, lung cancer, non-small cell lung cancer, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, pancreatic cancer, leukemia, acute myeloid leukemia (AML), chronic myeloid leukemia (PCML), acute lymphocytic leukemia (ALL), chronic lymphocytic leukemia (CLL), T-cell acute lymphoblastic leukemia, lymphoblastic disease, multiple myeloma, Hodgkin's lymphoma, non-Hodgkin's lymphoma, acne vulgaris, asthma, autoimmune diseases, autoinflammatory diseases, celiac disease, chronic prostatitis, glomerulonephritis, hypersensitivity reaction, inflammatory bowel disease, Crohn's disease, pelvic inflammatory disease, reperfusion injury, rheumatoid arthritis, sarcoidosis, transplant rejection, vasculitis, psoriasis, fibromyalgia, irritable bowel syndrome, lupus erythematosis, osteoarthritis, scleroderma, and ulcerative colitis.

126. The pharmaceutical composition of claim 124 for use in a pharmaceutical regimen for treatment of a disease in a subject.

127. The pharmaceutical composition of claim 126, wherein the pharmaceutical regimen further comprises the step of determining the amount of pharmaceutical composition needed to achieve a beneficial effect in the subject having the disease.

128. A method of treating a disease in a subject, comprising administering a therapeutically effective dose of the pharmaceutical composition of claim 124 to a subject in need thereof.

129. The method of claim 128, wherein the disease is selected from the group consisting of breast cancer, ER/PR+ breast cancer, Her2+ breast cancer, triple-negative breast cancer, liver carcinoma, lung cancer, non-small cell lung cancer, colorectal cancer, esophageal carcinoma, fibrosarcoma, choriocarcinoma, ovarian cancer, cervical carcinoma, laryngeal carcinoma, endometrial carcinoma, hepatocarcinoma, gastric cancer, prostate cancer, renal cell carcinoma, Kaposi's sarcoma, astrocytoma, melanoma, squamous cell cancer, basal cell carcinoma, head and neck cancer, thyroid carcinoma, Wilm's tumor, urinary tract carcinoma, thecoma, arrhenoblastoma, glioblastomoa, and pancreatic cancer.

130. The method of claim 129, wherein the first targeting moiety has specific binding affinity for a tumor of the disease.

131. The method of claim 129, wherein the first targeting moiety has specific binding affinity for a target set forth in Table 2, Table 3, Table 4, Table 18, and Table 19.

132. The method of claim 129, wherein administering the therapeutically effective dose results in at least a 10% improvement of a parameter associated with the disease compared to an untreated subject, wherein the parameter is selected from the group consisting of time-to-progression of the disease, time-to-relapse, time-to-discovery of local recurrence, time-to-discovery of regional metastasis, time-to-discovery of distant metastasis, time-to-onset of symptoms, pain, body weight, hospitalization, time-to-increase in pain medication requirement, time-to-requirement of salvage chemotherapy, time-to-requirement of salvage surgery, time-to-requirement of salvage radiotherapy, time-to-treatment failure, and time of survival.

133. The method of claim 130, wherein administering the therapeutically effective dose results in a decrease in size of the tumor of the disease.

134. The method of claim 133, wherein the decrease in size of the tumor is at least a 10% decrease.

135. The method of claim 133, wherein the decrease in size of the tumor is achieved within at least about 10 days after administering the therapeutically effective dose.

136. The method of claim 130, wherein administering the therapeutically effective dose results in tumor stasis in the subject.

137. The method of claim 136, wherein tumor stasis is achieved within at least about 21 days after administering the therapeutically effective dose.

138. The method of claim 128, comprising administering a therapeutically effective dose every 7 days, every 10 days, every 14 days, every 21 days, or every 30 days.

139. The method of claim 128, wherein the recombinant polypeptide of the pharmaceutical composition exhibits a terminal half-life that is longer than at least about 72 h when administered to the subject.

140. An isolated nucleic acid comprising a polynucleotide sequence selected from (a) a polynucleotide encoding the recombinant polypeptide of claim 111 and (b) the complement of the polynucleotide of (a).

141. An expression vector comprising the polynucleotide sequence of claim 140 and a recombinant regulatory sequence operably linked to the polynucleotide sequence.

142. An isolated host cell, comprising the expression vector of claim 141.

143. The host cell of claim 142, wherein the host cell is E. coli.
